(19) United States
(12) Patent Application Publication
(10) Pub. No.: US 2025/0249079 A1
(43) Pub. Date: Aug. 7, 2025

(54) COMPOSITIONS AND METHODS FOR THE TREATMENT OF ACTINOMYCETIA INFECTIONS

(71) Applicant: Endolytix Technology, Inc., Beverly, MA (US)

(72) Inventors: Jason HOLDER, Swampscott, MA (US); Helen BARTLETT, Somerville, MA (US); Sonia BARRIOS, Malden, MA (US); Clinton C. DAWSON, Amesbury, MA (US); Keith SOLOMON, Hyde Park, MA (US); Cody GLICKMAN, Englewood, CO (US)

(21) Appl. No.: 18/855,101

(22) PCT Filed: Apr. 7, 2023
(86) PCT No.: PCT/US2023/065497

§ 371 Date: Oct. 8, 2024

Related U.S. Application Data
(60) Provisional application No. 63/429,869, filed on Dec. 2, 2022.
(60) Provisional application No. 63/329,276, filed on Apr. 8, 2022.

(51) Int. Cl. A61K 38/47; C12N 9/26; C12N 9/36; C12N 9/44

(52) U.S. Cl. CPC A61K 38/47; C12N 9/2414; C12N 9/246; C12N 9/2462; C12Y 302/01001; C12Y 302/01017; C12Y 302/01068

(57) ABSTRACT

The present disclosure features compositions and methods for the treatment of actinomycetia (e.g., corynebacteriales) infections, e.g., caused by mycobacterial cells, residing within a host cell (e.g., a mammalian cell, e.g., immune cell, e.g., macrophage or dendritic cell). The compositions and methods include delivering a mixture of lytic enzymes to specifically target the cell envelope of a group of related bacterial species in the intracellular compartment (endosome, phagosome, lysosome, or cytosol) in which above mentioned orders of bacteria infect, grow, hide, and go quiescent.

### SEQUENCE LISTING

The instant application contains a Sequence Listing which has been submitted electronically in XML format and is hereby incorporated by reference in its entirety. Said XML copy, created on Apr. 5, 2023, is named 51486-005WO3_Sequence_Listing_4_5_23.XML and is 660,322 bytes in size.

### BACKGROUND

Bacterial pathogens are a leading cause of infectious disease. Many bacteria are successfully detected by the human immune system and are rapidly cleared before onset of infection. However, many bacterial pathogens evade the host immune system by residing within a host cell. These intracellular bacteria have evolved diverse immune evasion techniques by residing and multiplying within host cells, such as immune cells (e.g., macrophages or dendritic cells), and the correct intracellular compartment (e.g., endosome, phagosome, lysosome, or cytosol) within the host cells. Bacterial infections that propagate within a host cell often present a difficult treatment barrier due to lack of accessibility of the subcellular location of the infection. While certain anti-bacterial compositions may treat the infection (e.g., in vitro), delivering the treatment to the correct subcellular location in which the bacteria reside has proved to be a challenging endeavor.

One group of challenging intracellular bacterial infections is caused by mycobacteria. Mycobacteria are actinomycetia (e.g., corynebacteriales or propionibacteriales) that are denoted by a thick cell wall that is rich in mycolic acids. Mycobacteria contain an envelope that contains a cell membrane composed of a lipid bilayer and cell wall that includes a peptidoglycan layer and an arabinogalactan layer, and outer membrane that contains a hydrophobic mycolate layer. Many mycobacteria also contain an outer capsule composed of polysaccharides, such as D-glucan, D-arabino-D-mannan, and D-mannan. This complex cell envelope contributes to the hardiness of the mycobacteria and is particularly difficult to penetrate and destroy. Pathogenic mycobacteria are often partitioned into two groups: M. tuberculosis and non-tuberculosis mycobacteria (NTM). In contrast to tuberculosis, person-to-person transmission of NTM is rare. Nonetheless, the number of NTM infections is a growing health concern, particularly in people with lung disease.

Improved compositions and methods for targeting and treating intracellular bacterial infections, such as those caused by mycobacteria, are needed.

### SUMMARY OF THE INVENTION

In one aspect, the invention features a composition that includes a supramolecular structure including one or more (e.g., two or more, three or more, or all four of) (a) a Lysin A that includes an amino acid sequence having at least 85% (e.g., at least 90%, 95%, 97%, 99%, or 100%) sequence identity to any one of SEQ ID NOS: 1-182; (b) a Lysin B that includes an amino acid sequence having at least 85% (e.g., at least 90%, 95%, 97%, 99%, or 100%) sequence identity to any one of SEQ ID NOs: 183-241; (c) an isoamylase that includes an amino acid sequence having at least 85% (e.g., at least 90%, 95%, 97%, 99%, or 100%) sequence identity to any one of SEQ ID NOs: 242-392; and (d) an α-amylase including an amino acid sequence having at least 85% (e.g., at least 90%, 95%, 97%, 99%, or 100%) sequence identity to any one of SEQ ID NOS: 393-445. In some embodiments, the invention features a composition that includes a supramolecular structure including one or more (e.g., two or more, three or more, or all four of) (a) a Lysin A that includes an amino acid sequence having at least 85% (e.g., at least 90%, 95%, 97%, 99%, or 100%) sequence identity to SEQ ID NO: 1 or SEQ ID NO: 2; (b) a Lysin B that includes an amino acid sequence having at least 85% (e.g., at least 90%, 95%, 97%, 99%, or 100%) sequence identity to SEQ ID NO: 183 or SEQ ID NO: 184; (c) an isoamylase that includes an amino acid sequence having at least 85% (e.g., at least 90%, 95%, 97%, 99%, or 100%) sequence identity to SEQ ID NO: 242 or SEQ ID NO: 243; and (d) an α-amylase including an amino acid sequence having at least 85% (e.g., at least 90%, 95%, 97%, 99%, or 100%) sequence identity to any one of SEQ ID NOs: 393-398.

In some embodiments, the composition includes Lysin A and Lysin B, and the Lysin A includes an amino acid sequence having at least 85% (e.g., at least 90%, 95%, 97%, 99%, or 100%) sequence identity to any one of SEQ ID NOs: 1-182; and the Lysin B includes an amino acid sequence having at least 85% (e.g., at least 90%, 95%, 97%, 99%, or 100%) sequence identity to any one of SEQ ID NOS: 183-241.

In some embodiments, the composition includes Lysin A and Lysin B, and the Lysin A includes an amino acid sequence having at least 85% (e.g., at least 90%, 95%, 97%, 99%, or 100%) sequence identity to SEQ ID NO: 1 or SEQ ID NO: 2; and the Lysin B includes an amino acid sequence having at least 85% (e.g., at least 90%, 95%, 97%, 99%, or 100%) sequence identity to SEQ ID NO: 183 or SEQ ID NO: 184.

In some embodiments, the composition includes Lysin A and isoamylase, and the Lysin A includes an amino acid sequence having at least 85% (e.g., at least 90%, 95%, 97%, 99%, or 100%) sequence identity to any one of SEQ ID NOS: 1-182; and the isoamylase includes an amino acid sequence having at least 85% (e.g., at least 90%, 95%, 97%, 99%, or 100%) sequence identity to any one of SEQ ID NOS: 242-392.

In some embodiments, the composition includes Lysin A and isoamylase, and the Lysin A includes an amino acid sequence having at least 85% (e.g., at least 90%, 95%, 97%, 99%, or 100%) sequence identity to SEQ ID NO: 1 or SEQ ID NO: 2; and the isoamylase includes an amino acid sequence having at least 85% (e.g., at least 90%, 95%, 97%, 99%, or 100%) sequence identity to SEQ ID NO: 242 or SEQ ID NO: 243.

In some embodiments, the composition includes Lysin A and α-amylase, and the Lysin A includes an amino acid sequence having at least 85% (e.g., at least 90%, 95%, 97%, 99%, or 100%) sequence identity to any one of SEQ ID NOs: 1-182; and the α-amylase includes an amino acid sequence having at least 85% (e.g., at least 90%, 95%, 97%, 99%, or 100%) sequence identity to any one of SEQ ID NOS: 393-445.

In some embodiments, the composition includes Lysin A and α-amylase, and the Lysin A includes an amino acid sequence having at least 85% (e.g., at least 90%, 95%, 97%, 99%, or 100%) sequence identity to SEQ ID NO: 1 or SEQ ID NO: 2; and the α-amylase includes an amino acid sequence having at least 85% (e.g., at least 90%, 95%, 97%, 99%, or 100%) sequence identity to any one of SEQ ID NOS: 393-398.

In some embodiments, the composition includes Lysin B and isoamylase, and the Lysin B includes an amino acid sequence having at least 85% (e.g., at least 90%, 95%, 97%, 99%, or 100%) sequence identity to any one of SEQ ID NOs: 183-241; and the isoamylase includes an amino acid sequence having at least 85% (e.g., at least 90%, 95%, 97%, 99%, or 100%) sequence identity to any one of SEQ ID NOs: 242-392.

In some embodiments, the composition includes Lysin B and isoamylase, and the Lysin B includes an amino acid sequence having at least 85% (e.g., at least 90%, 95%, 97%, 99%, or 100%) sequence identity to SEQ ID NO: 183 or SEQ ID NO: 184; and the isoamylase includes an amino acid sequence having at least 85% (e.g., at least 90%, 95%, 97%, 99%, or 100%) sequence identity to SEQ ID NO: 242 or SEQ ID NO: 243.

In some embodiments, the composition includes Lysin B and α-amylase, and the Lysin B includes an amino acid sequence having at least 85% (e.g., at least 90%, 95%, 97%, 99%, or 100%) sequence identity to any one of SEQ ID NOs: 183-241; and the α-amylase includes an amino acid sequence having at least 85% (e.g., at least 90%, 95%, 97%, 99%, or 100%) sequence identity to any one of SEQ ID NOS: 393-445.

In some embodiments, the composition includes Lysin B and α-amylase, and the Lysin B includes an amino acid sequence having at least 85% (e.g., at least 90%, 95%, 97%, 99%, or 100%) sequence identity to SEQ ID NO: 183 or SEQ ID NO: 184; and the α-amylase includes an amino acid sequence having at least 85% (e.g., at least 90%, 95%, 97%, 99%, or 100%) sequence identity to any one of SEQ ID NOs: 393-398.

In some embodiments, the composition includes isoamylase and α-amylase, and the isoamylase includes an amino acid sequence having at least 85% (e.g., at least 90%, 95%, 97%, 99%, or 100%) sequence identity to any one of SEQ ID NOs: 242-392; and the α-amylase includes an amino acid sequence having at least 85% (e.g., at least 90%, 95%, 97%, 99%, or 100%) sequence identity to any one of SEQ ID NOs: 393-445.

In some embodiments, the composition includes isoamylase and α-amylase, and the isoamylase includes an amino acid sequence having at least 85% (e.g., at least 90%, 95%, 97%, 99%, or 100%) sequence identity to SEQ ID NO: 242 or SEQ ID NO: 243; and the α-amylase includes an amino acid sequence having at least 85% (e.g., at least 90%, 95%, 97%, 99%, or 100%) sequence identity to any one of SEQ ID NOs: 393-398.

In some embodiments, the composition includes Lysin A, Lysin B, and isoamylase, and the Lysin A includes an amino acid sequence having at least 85% (e.g., at least 90%, 95%, 97%, 99%, or 100%) sequence identity to any one of SEQ ID NOS: 1-182; the Lysin B includes an amino acid sequence having at least 85% (e.g., at least 90%, 95%, 97%, 99%, or 100%) sequence identity to any one of SEQ ID NOS: 183-241; and the isoamylase includes an amino acid sequence having at least 85% (e.g., at least 90%, 95%, 97%, 99%, or 100%) sequence identity to any one of SEQ ID NOs: 242-392.

In some embodiments, the composition includes Lysin A, Lysin B, and isoamylase, and the Lysin A includes an amino acid sequence having at least 85% (e.g., at least 90%, 95%, 97%, 99%, or 100%) sequence identity to SEQ ID NO: 1 or SEQ ID NO: 2; the Lysin B includes an amino acid sequence having at least 85% (e.g., at least 90%, 95%, 97%, 99%, or 100%) sequence identity to SEQ ID NO: 183 or SEQ ID NO: 184; and the isoamylase includes an amino acid sequence having at least 85% (e.g., at least 90%, 95%, 97%, 99%, or 100%) sequence identity to SEQ ID NO: 242 or SEQ ID NO: 243.

In some embodiments, the composition includes Lysin A, Lysin B, and α-amylase, and the Lysin A includes an amino acid sequence having at least 85% (e.g., at least 90%, 95%, 97%, 99%, or 100%) sequence identity to any one of SEQ ID NOS: 1-182; the Lysin B includes an amino acid sequence having at least 85% (e.g., at least 90%, 95%, 97%, 99%, or 100%) sequence identity to any one of SEQ ID NOS: 183-241; and the α-amylase includes an amino acid sequence having at least 85% (e.g., at least 90%, 95%, 97%, 99%, or 100%) sequence identity to any one of SEQ ID NOs: 393-445.

In some embodiments, the composition includes Lysin A, Lysin B, and α-amylase, and the Lysin A includes an amino acid sequence having at least 85% (e.g., at least 90%, 95%, 97%, 99%, or 100%) sequence identity to SEQ ID NO: 1 or SEQ ID NO: 2; the Lysin B includes an amino acid sequence having at least 85% (e.g., at least 90%, 95%, 97%, 99%, or 100%) sequence identity to SEQ ID NO: 183 or SEQ ID NO: 184; and the α-amylase includes an amino acid sequence having at least 85% (e.g., at least 90%, 95%, 97%, 99%, or 100%) sequence identity to any one of SEQ ID NOs: 393-398.

In some embodiments, the composition includes Lysin A, isoamylase, and α-amylase, and the Lysin A includes an amino acid sequence having at least 85% (e.g., at least 90%, 95%, 97%, 99%, or 100%) sequence identity to any one of SEQ ID NOS: 1-182; the isoamylase includes an amino acid sequence having at least 85% (e.g., at least 90%, 95%, 97%, 99%, or 100%) sequence identity to any one of SEQ ID NOs: 242-392; and the α-amylase includes an amino acid sequence having at least 85% (e.g., at least 90%, 95%, 97%, 99%, or 100%) sequence identity to any one of SEQ ID NOs: 393-445.

In some embodiments, the composition includes Lysin A, isoamylase, and α-amylase, and the Lysin A includes an amino acid sequence having at least 85% (e.g., at least 90%, 95%, 97%, 99%, or 100%) sequence identity to SEQ ID NO: 1 or SEQ ID NO: 2; the isoamylase includes an amino acid sequence having at least 85% (e.g., at least 90%, 95%, 97%, 99%, or 100%) sequence identity to SEQ ID NO: 242 or SEQ ID NO: 243; and the α-amylase includes an amino acid sequence having at least 85% (e.g., at least 90%, 95%, 97%, 99%, or 100%) sequence identity to any one of SEQ ID NOs: 393-398.

In some embodiments, the composition includes Lysin B, isoamylase, and α-amylase, and the Lysin B includes an amino acid sequence having at least 85% (e.g., at least 90%, 95%, 97%, 99%, or 100%) sequence identity to any one of SEQ ID NOs: 183-241; the isoamylase includes an amino acid sequence having at least 85% (e.g., at least 90%, 95%, 97%, 99%, or 100%) sequence identity to any one of SEQ ID NOs: 242-392; and the α-amylase includes an amino acid sequence having at least 85% (e.g., at least 90%, 95%, 97%, 99%, or 100%) sequence identity to any one of SEQ ID NOs: 393-445.

In some embodiments, the composition includes Lysin B, isoamylase, and α-amylase, and the Lysin B includes an amino acid sequence having at least 85% (e.g., at least 90%, 95%, 97%, 99%, or 100%) sequence identity to SEQ ID NO: 183 or SEQ ID NO: 184; the isoamylase includes an amino acid sequence having at least 85% (e.g., at least 90%, 95%, 97%, 99%, or 100%) sequence identity to SEQ ID NO: 242 or SEQ ID NO: 243; and the α-amylase includes an amino acid sequence having at least 85% (e.g., at least 90%, 95%, 97%, 99%, or 100%) sequence identity to any one of SEQ ID NOs: 393-398.

In some embodiments, the composition includes Lysin A, Lysin B, isoamylase, and α-amylase, and the Lysin A includes an amino acid sequence having at least 85% (e.g., at least 90%, 95%, 97%, 99%, or 100%) sequence identity to any one of SEQ ID NOS: 1-182; the Lysin B includes an amino acid sequence having at least 85% (e.g., at least 90%, 95%, 97%, 99%, or 100%) sequence identity to any one of SEQ ID NOs: 183-241; the isoamylase includes an amino acid sequence having at least 85% (e.g., at least 90%, 95%, 97%, 99%, or 100%) sequence identity to any one of SEQ ID NOs: 242-392; and the α-amylase includes an amino acid sequence having at least 85% (e.g., at least 90%, 95%, 97%, 99%, or 100%) sequence identity to any one of SEQ ID NOS: 393-445.

In some embodiments, the composition includes Lysin A, Lysin B, isoamylase, and α-amylase, and the Lysin A includes an amino acid sequence having at least 85% (e.g., at least 90%, 95%, 97%, 99%, or 100%) sequence identity to SEQ ID NO: 1 or SEQ ID NO: 2; the Lysin B includes an amino acid sequence having at least 85% (e.g., at least 90%, 95%, 97%, 99%, or 100%) sequence identity to SEQ ID NO: 183 or SEQ ID NO: 184; the isoamylase includes an amino acid sequence having at least 85% (e.g., at least 90%, 95%, 97%, 99%, or 100%) sequence identity to SEQ ID NO: 242 or SEQ ID NO: 243; and the α-amylase includes an amino acid sequence having at least 85% (e.g., at least 90%, 95%, 97%, 99%, or 100%) sequence identity to any one of SEQ ID NOs: 393-398.

In some embodiments, the composition includes Lysin A, Lysin B, isoamylase, and α-amylase, and the Lysin A includes the amino acid sequence of SEQ ID NO: 1 or SEQ ID NO: 2; the Lysin B includes the amino acid sequence of SEQ ID NO: 183 or SEQ ID NO: 184; the isoamylase includes the amino acid sequence of SEQ ID NO: 242 or SEQ ID NO: 243; and the α-amylase includes the amino acid sequence of any one of SEQ ID NOs: 393-398.

In some embodiments, the composition includes a Lysin A of SEQ ID NO: 1, a Lysin B of SEQ ID NO: 183, an isoamylase of SEQ ID NO: 242, and an α-amylase of SEQ ID NO: 393. In some embodiments, the composition includes a Lysin A of SEQ ID NO: 2, a Lysin B of SEQ ID NO: 184, an isoamylase of SEQ ID NO: 243, and an α-amylase of SEQ ID NO: 394.

In some embodiments, the composition includes a Lysin A of SEQ ID NO: 1, a Lysin B of SEQ ID NO: 183, an isoamylase of SEQ ID NO: 242, and an α-amylase of SEQ ID NO: 395. In some embodiments, the composition includes a Lysin A of SEQ ID NO: 2, a Lysin B of SEQ ID NO: 184, an isoamylase of SEQ ID NO: 243, and an α-amylase of SEQ ID NO: 396.

In some embodiments, the composition includes a Lysin A of SEQ ID NO: 1, a Lysin B of SEQ ID NO: 183, an isoamylase of SEQ ID NO: 242, and an α-amylase of SEQ ID NO: 397. In some embodiments, the composition includes a Lysin A of SEQ ID NO: 2, a Lysin B of SEQ ID NO: 184, an isoamylase of SEQ ID NO: 243, and an α-amylase of SEQ ID NO: 398.

In some embodiments of any of the above aspects, the Z-average mean particle diameter of the supramolecular structure is from 75 nm to 5 μm, e.g., from 75 nm to 2 μm, from 75 nm to 1 μm, e.g., from 75 nm to 750 nm (e.g., from 250 nm to 750 nm, or from 75 nm to 250 nm). In some embodiments, when the supramolecular structure is an LNP or micelle, the Z-average mean particle diameter is from 75 nm to 250 nm. In some embodiments, when the supramolecular structure is a vesicle (e.g., a liposome), the Z-average mean particle diameter is from 250 nm to 750 nm. Non-limiting examples of the Z-average mean particle diameters include, e.g., from 75 nm to 100 nm, e.g., from 75 nm to 85 nm, e.g., 80 nm, e.g., from 80 nm to 140 nm, from 90 nm to 130 nm, or from 110 nm to 130 nm, e.g., 120 nm, e.g., from 200 nm to 300 nm, e.g., from 250 nm to 300 nm, from 260 nm to 290 nm, from 260 nm to 280 nm, from 265 nm to 275 nm, e.g., 270 nm, e.g., from 300 nm to 400 nm, from 400 nm to 600 nm, e.g., from 450 nm to 550 nm, from 475 nm to 525 nm, from 480 nm to 520 nm, from 490 nm to 510 nm, from 495 nm to 505 nm, e.g., 500 nm, e.g., 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485 nm, 490 nm, 495 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575 nm, 580 nm, 585 nm, 590 nm, 595 nm, 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675 nm, 680 nm, 685 nm, 690 nm, 695 nm, 700 nm, 705 nm, 710 nm, 715 nm, 720 nm, 725 nm, 730 nm, 735 nm, 740 nm, 745 nm, 750 nm, 755 nm, 760 nm, 765 nm, 770 nm, 775 nm, 780 nm, 785 nm, 790 nm, 795 nm, 800 nm, 805 nm, 810 nm, 815 nm, 820 nm, 825 nm, 830 nm, 835 nm, 840 nm, 845 nm, 850 nm, 855 nm, 860 nm, 865 nm, 870 nm, 875 nm, 880 nm, 885 nm, 890 nm, 895 nm, 900 nm, 905 nm, 910 nm, 915 nm, 920 nm, 925 nm, 930 nm, 935 nm, 940 nm, 945 nm, 950 nm, 955 nm, 960 nm, 965 nm, 970 nm, 975 nm, 980 nm, 985 nm, 990 nm, 995 nm, 1 μm, 1.1 μm, 1.2 μm, 1.3 μm, 1.4 μm, 1.5 μm, 1.6 μm, 1.7 μm, 1.8 μm, 1.9 μm, 2 μm, 2.1 μm, 2.2 μm 2.3 μm, 2.4 μm, 2.5 μm, 2.6 μm, 2.7 μm, 2.8 μm, 2.9 μm, 3 μm, 3.1 μm, 3.2 μm, 3.3 μm, 3.4 μm, 3.5 μm, 3.6 μm, 3.7 μm, 3.8 μm, 3.9 μm, 4 μm, 4.1 μm 4.2 μm 4.3 μm, 4.4 μm, 4.5 μm, 4.6 μm, 4.7 μm, 4.8 μm, 4.9 μm, or 5 μm. In some embodiments, the Z-average mean particle diameter of the supramolecular structure is 80 nm, 270 nm, or 500 nm. In some embodiments, the supramolecular structure includes a Z-average mean particle diameter of from 75 nm to 750 nm. In some embodiments, the Z-average mean particle diameter is from 250 nm to 750 nm. In some embodiments, the Z-average mean particle diameter is from 75 nm to 250 nm.

In some embodiments, the supramolecular structure is a is a lipid nanoparticle.

In some embodiments, the supramolecular structure is a micelle.

In some embodiments, the supramolecular structure is a liposome. The liposome may be unilamellar. Alternatively, the liposome may be multilamellar.

In some embodiments, the supramolecular structure includes polydispersity index of from 0.05 to 0.3.

In some embodiments, the supramolecular structure includes one or more lipids. In some embodiments, at least one of the one or more lipids may be, for example, an ionizable lipid.

In some embodiments, the lipid may be, for example, 1,2-dioleoyl-sn-glycero-3-phosphocholine (DOPC), 1,2-dioleoyl-sn-glycero-3-phosphoethanolamine (DOPE), or 1,2-dioleoyl-sn-glycero-3-phospho-L-serine (DOPS).

In some embodiments, the lipid is a sterol, e.g., cholesterol or a derivative thereof.

In some embodiments, the composition includes a mixture of lipids. For example, the mixture of lipids may include two or more of DOPC, DOPE, DOPS, and cholesterol.

In some embodiments, the DOPC and DOPE are present at a molar ration of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPC and DOPS are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPC and cholesterol are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPE and DOPS are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPE and cholesterol are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPS and cholesterol are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPC, DOPE, DOPS, and cholesterol are present at a molar ratio of from 1-20:1-20:1-5:1-5. For example, in some embodiments, the DOPC, DOPE, DOPS, and cholesterol are present at a molar ratio of 10:10:3:4.

In some embodiments, the composition includes a concentration of lipids of from 0.03 mg/mL to mg/mL, e.g., 0.1 mg/mL to 10 mg/ml (e.g., from 0.1 mg/mL to 1 mg/mL, e.g., 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, or 1 mg/mL, e.g., from 1 mg/mL to 10 mg/mL, e.g., 2 mg/ml, 3 mg/mL, 4 mg/mL, 5 mg/mL, 6 mg/mL, 7 mg/mL, 8 mg/mL, 9 mg/mL, or 10 mg/mL). In some embodiments, the composition includes a concentration of lipids of from 1 mg/ml to 5 mg/mL.

In some embodiments, the composition includes a concentration of proteins (e.g., Lysin A, Lysin B, isoamylase, and/or α-amylase) of from 0.1 mg/ml to 20 mg/ml (e.g., e.g., from 0.1 mg/mL to 1 mg/mL, e.g., 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, or 1 mg/mL, e.g., from 1 mg/mL to 10 mg/mL, e.g., 2 mg/ml, 3 mg/mL, 4 mg/mL, 5 mg/mL, 6 mg/mL, 7 mg/mL, 8 mg/mL, 9 mg/mL, 10 mg/mL, e.g., from 10 mg/mL to 20 mg/mL, e.g., 11 mg/mL, 12 mg/mL, 13 mg/mL, 14 mg/mL, 15 mg/mL, 16 mg/mL, 17 mg/mL, 18 mg/mL, 19 mg/mL, or 20 mg/mL). In some embodiments, the composition includes a concentration of Lysin A, Lysin B, isoamylase, and/or α-amylase of from 1 mg/ml to 10 mg/mL.

In some embodiments, the compositions described herein are formulated with one or more buffers and/or excipients. For example, the composition (e.g., a supramolecular structure, e.g., a liposome containing a cocktail of lytic enzymes) may be encapsulated and/or formulated in buffer, such as glycine, Tris, sodium citrate, sodium acetate, and MES, e.g., at a concentration of 10 mM to 200 mM, e.g., 50 mm to 150 mm, e.g., 10 mM, 20 mm, 30 mm, 40 mm, 50 mm, 60 mm, 70 mm, 80 mm, 90 mM, 100 mM, 110 mM, 120 mM, 130 mM, 140 mM, 150 mM, 160 mM, 170 mM, 180 mM, 190 mM, or 200 mM. The composition may be formulated at a pH of from 5 to 11 (e.g., a pH of 5 to 6, e.g., 5.1, 5.2, 5.3, 5.4, 5.5, 5.6, 5.7, 5.8, 5.9, or 6, e.g., 6 to 11, e.g., 6.5, 7, 7.5, 8, 8.5, 9, 9.5, 10, 10.5, or 11). The composition may further include one or more excipients, such as CaCl₂), arginine, NaCl, sodium citrate, MgCl₂, or glycerol. In some embodiments, the composition includes, for example, 5 mM to 10 mM CaCl₂), 0 to 50 mM arginine, 0 to 200 mM NaCl, 0 to 1 mM sodium citrate, 0 to 1 mM MgCl₂, and/or 10-30% glycerol. In some embodiments, the composition includes 50 mM glycine, pH 8.5, 7.5 mM CaCl₂), 0.5 mM MgCl₂, 200 mM NaCl, 0.33 mM sodium citrate, and 10% glycerol. The formulation may further include TWEEN, e.g., TWEEN-80.

In some embodiments, the composition further includes a targeting moiety. The targeting moiety may be, for example, an extracellular targeting moiety targeting a professional antigen presenting cell (e.g., a macrophage or a dendritic cell). In some embodiments, the targeting moiety is phosphatidylserine.

In another aspect, the invention features a method of treating a bacterial infection in a subject. The method includes administering a composition as described herein, e.g., of any of the above embodiments, to the subject in an amount and for a duration sufficient to treat the bacterial infection.

In some embodiments the bacterial infection is caused by an actinomycetia bacterium. In some embodiments, the actinomycetia is a corynebacteriales or propionibacteriales. In some embodiments, the cornyebacteriales is a Mycobacterium species.

In some embodiments, the Mycobacterium species is M. tuberculosis, M. leprae, M. lepromatosis, M. avium, M. kansasii, M. fortuitum, M. chelonae, M. marinum, M. intracellulare, M. abscessus, M. chimera, M. boletti, M. fortuitum, M. goodii, or M. masiliense.

In some embodiments, the corynebacteriales is a Nocardia, Corynebacterium, or Rhodococcus species.

In some embodiments, the propionibacteriales is a Cutibacterium species.

For example, in some embodiments, the compositions and methods described herein may be used to target other actinomycetia (e.g., corynebacteriales or propionibacteriales) that have similar envelope components as mycobacteria. For example, the compositions and methods may be used to target a Nocardia, Corynebacterium, or Rhodococcus species. For example, the Nocardia species may be, e.g., N. brasiliensis, N. cyriacigeorgica, N. farcinica, N. nova, N. asteroids, N. brasiliensis, and N. caviae. The Corynebacterium species may be, e.g., C. glutamicum or C. diphtheriae. The Rhodococcus species may be, e.g., R. fascians or R. equi.

In some embodiments, the compositions and methods may be used to target a Cutibacterium species. The Cutibacterium species may be, e.g., C. acnes.

In some embodiments, the method further includes administering an antibiotic. In some embodiments, the antibiotic is a cephalosporin, a carbapenem, a penicillin, an aminoglycoside, a cephalosporin, a rifamycin, a macrolide, or a fluoroquinolone. In some embodiments, the antibiotic is thiacetazone, sq-109, bedaquiline, delamanid, pyrazinamide, or isoniazid. In some embodiments, the antibiotic is azithromycin, clarithromycin, ethambutol, rifampin, biapenem, or amikacin. In some embodiments, the antibiotic is a macrolide (e.g., azithromycin, clarithromycin, erythromycin). In some embodiments, the antibiotic is a macrolide (e.g., azithromycin, clarithromycin, erythromycin). In some embodiments, the antibiotic is an aminoglycoside (e.g., kanamycin A, amikacin, tobramycin, dibekacin, gentamicin, sisomicin, netilmicin, neomycin (e.g., neomycin B, C, or E), streptomycin, or plazomicin).

In some embodiments, the composition is administered intravenously, orally, or via inhalation (e.g., via aerosol).

In another aspect, the invention features a method of assembly. The method includes providing a sample that includes two or more of (e.g., two, three, or four of) a Lysin A, a Lysin B, an isoamylase, and an α-amylase. The method further includes mixing the sample with one or more lipids in the presence of an organic solvent to form a liposome comprising the two or more of Lysin A, Lysin B, isoamylase, α-amylase.

In some embodiments, the Lysin A includes an amino acid sequence having at least 85% (e.g., at least 90%, 95%, 97%, 99%, or 100%) sequence identity to any one of SEQ ID NOS: 1-182; the Lysin B includes an amino acid sequence having at least 85% (e.g., at least 90%, 95%, 97%, 99%, or 100%) sequence identity to any one of SEQ ID NOs: 183-241; the isoamylase includes an amino acid sequence having at least 85% (e.g., at least 90%, 95%, 97%, 99%, or 100%) sequence identity to any one of SEQ ID NOs: 242-392; and/or the α-amylase includes an amino acid sequence having at least 85% (e.g., at least 90%, 95%, 97%, 99%, or 100%) sequence identity to any one of SEQ ID NOs: 393-445.

In some embodiments, the Lysin A includes an amino acid sequence having at least 85% (e.g., at least 90%, 95%, 97%, 99%, or 100%) sequence identity to SEQ ID NO: 1 or SEQ ID NO: 2; the a Lysin B includes an amino acid sequence having at least 85% (e.g., at least 90%, 95%, 97%, 99%, or 100%) sequence identity to SEQ ID NO: 183 or SEQ ID NO: 184; the isoamylase includes an amino acid sequence having at least 85% (e.g., at least 90%, 95%, 97%, 99%, or 100%) sequence identity to SEQ ID NO: 242 or SEQ ID NO: 243; and/or the α-amylase includes an amino acid sequence having at least 85% (e.g., at least 90%, 95%, 97%, 99%, or 100%) sequence identity to any one of SEQ ID NOs: 393-398.

In some embodiments, the composition includes Lysin A and Lysin B, and the Lysin A includes an amino acid sequence having at least 85% (e.g., at least 90%, 95%, 97%, 99%, or 100%) sequence identity to any one of SEQ ID NOs: 1-182; and the Lysin B includes an amino acid sequence having at least 85% (e.g., at least 90%, 95%, 97%, 99%, or 100%) sequence identity to any one of SEQ ID NOS: 183-241.

In some embodiments, the composition includes Lysin A and Lysin B, and the Lysin A includes an amino acid sequence having at least 85% (e.g., at least 90%, 95%, 97%, 99%, or 100%) sequence identity to SEQ ID NO: 1 or SEQ ID NO: 2; and the Lysin B includes an amino acid sequence having at least 85% (e.g., at least 90%, 95%, 97%, 99%, or 100%) sequence identity to SEQ ID NO: 183 or SEQ ID NO: 184.

In some embodiments, the composition includes Lysin A and isoamylase, and the Lysin A includes an amino acid sequence having at least 85% (e.g., at least 90%, 95%, 97%, 99%, or 100%) sequence identity to any one of SEQ ID NOs: 1-182; and the isoamylase includes an amino acid sequence having at least 85% (e.g., at least 90%, 95%, 97%, 99%, or 100%) sequence identity to any one of SEQ ID NOs: 242-392.

In some embodiments, the composition includes Lysin A and isoamylase, and the Lysin A includes an amino acid sequence having at least 85% (e.g., at least 90%, 95%, 97%, 99%, or 100%) sequence identity to SEQ ID NO: 1 or SEQ ID NO: 2; and the isoamylase includes an amino acid sequence having at least 85% (e.g., at least 90%, 95%, 97%, 99%, or 100%) sequence identity to SEQ ID NO: 242 or SEQ ID NO: 243.

In some embodiments, the composition includes Lysin A and α-amylase, and the Lysin A includes an amino acid sequence having at least 85% (e.g., at least 90%, 95%, 97%, 99%, or 100%) sequence identity to any one of SEQ ID NOs: 1-182; and the α-amylase includes an amino acid sequence having at least 85% (e.g., at least 90%, 95%, 97%, 99%, or 100%) sequence identity to any one of SEQ ID NOS: 393-445.

In some embodiments, the composition includes Lysin A and α-amylase, and the Lysin A includes an amino acid sequence having at least 85% (e.g., at least 90%, 95%, 97%, 99%, or 100%) sequence identity to SEQ ID NO: 1 or SEQ ID NO: 2; and the α-amylase includes an amino acid sequence having at least 85% (e.g., at least 90%, 95%, 97%, 99%, or 100%) sequence identity to any one of SEQ ID NOS: 393-398.

In some embodiments, the composition includes Lysin B and isoamylase, and the Lysin B includes an amino acid sequence having at least 85% (e.g., at least 90%, 95%, 97%, 99%, or 100%) sequence identity to any one of SEQ ID NOs: 183-241; and the isoamylase includes an amino acid sequence having at least 85% (e.g., at least 90%, 95%, 97%, 99%, or 100%) sequence identity to any one of SEQ ID NOs: 242-392.

In some embodiments, the composition includes Lysin B and isoamylase, and the Lysin B includes an amino acid sequence having at least 85% (e.g., at least 90%, 95%, 97%, 99%, or 100%) sequence identity to SEQ ID NO: 183 or SEQ ID NO: 184; and the isoamylase includes an amino acid sequence having at least 85% (e.g., at least 90%, 95%, 97%, 99%, or 100%) sequence identity to SEQ ID NO: 242 or SEQ ID NO: 243.

In some embodiments, the composition includes Lysin B and α-amylase, and the Lysin B includes an amino acid sequence having at least 85% (e.g., at least 90%, 95%, 97%, 99%, or 100%) sequence identity to any one of SEQ ID NOs: 183-241; and the α-amylase includes an amino acid sequence having at least 85% (e.g., at least 90%, 95%, 97%, 99%, or 100%) sequence identity to any one of SEQ ID NOS: 393-445.

In some embodiments, the composition includes Lysin B and α-amylase, and the Lysin B includes an amino acid sequence having at least 85% (e.g., at least 90%, 95%, 97%, 99%, or 100%) sequence identity to SEQ ID NO: 183 or SEQ ID NO: 184; and the α-amylase includes an amino acid sequence having at least 85% (e.g., at least 90%, 95%, 97%, 99%, or 100%) sequence identity to any one of SEQ ID NOs: 393-398.

In some embodiments, the composition includes isoamylase and α-amylase, and the isoamylase includes an amino acid sequence having at least 85% (e.g., at least 90%, 95%, 97%, 99%, or 100%) sequence identity to any one of SEQ ID NOs: 242-392; and the α-amylase includes an amino acid sequence having at least 85% (e.g., at least 90%, 95%, 97%, 99%, or 100%) sequence identity to any one of SEQ ID NOs: 393-445.

In some embodiments, the composition includes isoamylase and α-amylase, and the isoamylase includes an amino acid sequence having at least 85% (e.g., at least 90%, 95%, 97%, 99%, or 100%) sequence identity to SEQ ID NO: 242 or SEQ ID NO: 243; and the α-amylase includes an amino acid sequence having at least 85% (e.g., at least 90%, 95%, 97%, 99%, or 100%) sequence identity to any one of SEQ ID NOs: 393-398.

In some embodiments, the composition includes Lysin A, Lysin B, and isoamylase, and the Lysin A includes an amino acid sequence having at least 85% (e.g., at least 90%, 95%, 97%, 99%, or 100%) sequence identity to any one of SEQ ID NOS: 1-182; the Lysin B includes an amino acid sequence having at least 85% (e.g., at least 90%, 95%, 97%, 99%, or 100%) sequence identity to any one of SEQ ID NOS: 183-241; and the isoamylase includes an amino acid sequence having at least 85% (e.g., at least 90%, 95%, 97%, 99%, or 100%) sequence identity to any one of SEQ ID NOs: 242-392.

In some embodiments, the composition includes Lysin A, Lysin B, and isoamylase, and the Lysin A includes an amino acid sequence having at least 85% (e.g., at least 90%, 95%, 97%, 99%, or 100%) sequence identity to SEQ ID NO: 1 or SEQ ID NO: 2; the Lysin B includes an amino acid sequence having at least 85% (e.g., at least 90%, 95%, 97%, 99%, or 100%) sequence identity to SEQ ID NO: 183 or SEQ ID NO: 184; and the isoamylase includes an amino acid sequence having at least 85% (e.g., at least 90%, 95%, 97%, 99%, or 100%) sequence identity to SEQ ID NO: 242 or SEQ ID NO: 243.

In some embodiments, the composition includes Lysin A, Lysin B, and α-amylase, and the Lysin A includes an amino acid sequence having at least 85% (e.g., at least 90%, 95%, 97%, 99%, or 100%) sequence identity to any one of SEQ ID NOS: 1-182; the Lysin B includes an amino acid sequence having at least 85% (e.g., at least 90%, 95%, 97%, 99%, or 100%) sequence identity to any one of SEQ ID NOS: 183-241; and the α-amylase includes an amino acid sequence having at least 85% (e.g., at least 90%, 95%, 97%, 99%, or 100%) sequence identity to any one of SEQ ID NOs: 393-445.

In some embodiments, the composition includes Lysin A, Lysin B, and α-amylase, and the Lysin A includes an amino acid sequence having at least 85% (e.g., at least 90%, 95%, 97%, 99%, or 100%) sequence identity to SEQ ID NO: 1 or SEQ ID NO: 2; the Lysin B includes an amino acid sequence having at least 85% (e.g., at least 90%, 95%, 97%, 99%, or 100%) sequence identity to SEQ ID NO: 183 or SEQ ID NO: 184; and the α-amylase includes an amino acid sequence having at least 85% (e.g., at least 90%, 95%, 97%, 99%, or 100%) sequence identity to any one of SEQ ID NOs: 393-398.

In some embodiments, the composition includes Lysin A, isoamylase, and α-amylase, and the Lysin A includes an amino acid sequence having at least 85% (e.g., at least 90%, 95%, 97%, 99%, or 100%) sequence identity to any one of SEQ ID NOS: 1-182; the isoamylase includes an amino acid sequence having at least 85% (e.g., at least 90%, 95%, 97%, 99%, or 100%) sequence identity to any one of SEQ ID NOs: 242-392; and the α-amylase includes an amino acid sequence having at least 85% (e.g., at least 90%, 95%, 97%, 99%, or 100%) sequence identity to any one of SEQ ID NOs: 393-445.

In some embodiments, the composition includes Lysin A, isoamylase, and α-amylase, and the Lysin A includes an amino acid sequence having at least 85% (e.g., at least 90%, 95%, 97%, 99%, or 100%) sequence identity to SEQ ID NO: 1 or SEQ ID NO: 2; the isoamylase includes an amino acid sequence having at least 85% (e.g., at least 90%, 95%, 97%, 99%, or 100%) sequence identity to SEQ ID NO: 242 or SEQ ID NO: 243; and the α-amylase includes an amino acid sequence having at least 85% (e.g., at least 90%, 95%, 97%, 99%, or 100%) sequence identity to any one of SEQ ID NOs: 393-398.

In some embodiments, the composition includes Lysin B, isoamylase, and α-amylase, and the Lysin B includes an amino acid sequence having at least 85% (e.g., at least 90%, 95%, 97%, 99%, or 100%) sequence identity to any one of SEQ ID NOs: 183-241; the isoamylase includes an amino acid sequence having at least 85% (e.g., at least 90%, 95%, 97%, 99%, or 100%) sequence identity to any one of SEQ ID NOs: 242-392; and the α-amylase includes an amino acid sequence having at least 85% (e.g., at least 90%, 95%, 97%, 99%, or 100%) sequence identity to any one of SEQ ID NOs: 393-445.

In some embodiments, the composition includes Lysin B, isoamylase, and α-amylase, and the Lysin B includes an amino acid sequence having at least 85% (e.g., at least 90%, 95%, 97%, 99%, or 100%) sequence identity to SEQ ID NO: 183 or SEQ ID NO: 184; the isoamylase includes an amino acid sequence having at least 85% (e.g., at least 90%, 95%, 97%, 99%, or 100%) sequence identity to SEQ ID NO: 242 or SEQ ID NO: 243; and the α-amylase includes an amino acid sequence having at least 85% (e.g., at least 90%, 95%, 97%, 99%, or 100%) sequence identity to any one of SEQ ID NOs: 393-398.

In some embodiments, the composition includes Lysin A, Lysin B, isoamylase, and α-amylase, and the Lysin A includes an amino acid sequence having at least 85% (e.g., at least 90%, 95%, 97%, 99%, or 100%) sequence identity to any one of SEQ ID NOS: 1-182; the Lysin B includes an amino acid sequence having at least 85% (e.g., at least 90%, 95%, 97%, 99%, or 100%) sequence identity to any one of SEQ ID NOs: 183-241; the isoamylase includes an amino acid sequence having at least 85% (e.g., at least 90%, 95%, 97%, 99%, or 100%) sequence identity to any one of SEQ ID NOs: 242-392; and the α-amylase includes an amino acid sequence having at least 85% (e.g., at least 90%, 95%, 97%, 99%, or 100%) sequence identity to any one of SEQ ID NOs: 393-445.

In some embodiments, the composition includes Lysin A, Lysin B, isoamylase, and α-amylase, and the Lysin A includes an amino acid sequence having at least 85% (e.g., at least 90%, 95%, 97%, 99%, or 100%) sequence identity to SEQ ID NO: 1 or SEQ ID NO: 2; the Lysin B includes an amino acid sequence having at least 85% (e.g., at least 90%, 95%, 97%, 99%, or 100%) sequence identity to SEQ ID NO: 183 or SEQ ID NO: 184; the isoamylase includes an amino acid sequence having at least 85% (e.g., at least 90%, 95%, 97%, 99%, or 100%) sequence identity to SEQ ID NO: 242 or SEQ ID NO: 243; and the α-amylase includes an amino acid sequence having at least 85% (e.g., at least 90%, 95%, 97%, 99%, or 100%) sequence identity to any one of SEQ ID NOs: 393-398.

In some embodiments, the organic solvent comprises ethanol.

In some embodiments, the ethanol is provided at a ratio of 10:1 to 1:1 to an aqueous portion of the sample. For example, in some embodiments, the ratio is 8:1 to 3:1.

In some embodiments, the ethanol is provided at a ratio of 1:10 to 1:1 to an aqueous portion of the sample. For example, in some embodiments, the ratio is 1:8 to 1:3.

### Definitions

As used herein, the term “about” refers to +/−10% of a recited value.

As used herein, a “combination therapy” or “administered in combination” means that two (or more) agents or treatments are administered to a subject as part of a defined treatment regimen for a particular disease or condition. The treatment regimen defines the doses and periodicity of administration of each agent such that the effects of the separate agents on the subject overlap and/or allow for synergism. In some embodiments, the delivery of the two or more agents is simultaneous or concurrent, and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disease, is greater than what would be observed with one agent or treatment delivered alone or in the absence of the other. The effect of the two treatments can be partially additive, wholly additive, or greater than additive, e.g., synergistic. Sequential or substantially simultaneous administration of each therapeutic agent can be by any appropriate route including, but not limited to, oral routes, intravenous routes, intramuscular routes, topical routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the terms “effective amount,” “therapeutically effective amount,” and “sufficient amount” of an agent that results in a therapeutic effect, e.g., in a cell, sample, or subject, described herein refer to a quantity sufficient to, when administered to the cell, sample, or subject, including a human, effect beneficial or desired results, including pre-clinical or clinical results, and, as such, an “effective amount” or synonym thereto depends on the context in which it is being applied. For example, in the context of treating a disorder, it is an amount of the agent that is sufficient to achieve a treatment response as compared to the response obtained without administration of the agent. The amount of a given agent will vary depending upon various factors, such as the given agent, the pharmaceutical formulation, the route of administration, the severity of the bacterial infection, biomarkers, e.g., age, sex, and/or weight, of the subject, sample, or host cell, e.g., mammalian immune cell, being treated, and the like, but can nevertheless be routinely determined by one of ordinary skill in the art. Also, as used herein, the term “therapeutically effective amount” of an agent is an amount which results in a beneficial or desired result in a cell or subject as compared to a control. As defined herein, a therapeutically effective amount of an agent may be readily determined by one of ordinary skill by routine methods known in the art. Dosage regimen may be adjusted to provide the optimum therapeutic response.

The term “antibacterial lytic protein,” as used herein, refers to a protein that has bactericidal and/or bacteriolytic activity against bacteria. Non-limiting examples of antibacterial lytic proteins include holins, lysins (e.g., Lysin A and/or Lysin B), amylases (e.g., isoamylase or α-amylase), capsule depolymerases (e.g., hydrolase, metallohydrolase, epoxide hydrolase, peptidoglycan hydrolase, polysaccharase, polysaccharide lyase, endosialidase, hyaluronan lyase, or alginate lyase), beta lactamases, and lysozyme.

As used herein, “lipid nanoparticle” or “LNP” is a vesicle that includes a lipid layer encapsulating a substantially solid lipid core; the lipid core can contain a pharmaceutically active molecule. LNPs typically contain a cationic lipid, a non-cationic lipid, and a lipid that prevents aggregation of the particle (e.g., a PEG-lipid conjugate).

As used herein, the term “liposome” refers to a vesicle composed of amphiphilic lipids arranged in at least one bilayer, e.g., one bilayer or a plurality of bilayers. Liposomes include unilamellar and multilamellar (e.g., 2, 3, 4, 5, or more lamella) vesicles that have a membrane formed from a lipophilic material and an aqueous interior. The aqueous portion contains an antibacterial lytic protein or mixture of an antibacterial lytic protein and other components. The lipophilic material isolates the aqueous interior from an aqueous exterior, which typically does not include the phage protein, although in some examples, it may. Liposomes also include “sterically stabilized” liposomes, a term which, as used herein, refers to liposomes that include one or more specialized lipids that, when incorporated into liposomes, result in enhanced circulation lifetimes relative to liposomes lacking such specialized lipids.

“Micelles” are defined herein as a particular type of substantially spherical supramolecular structure in which amphiphilic molecules, e.g., lipids, are arranged such that the hydrophobic portions of the molecules are directed inward toward the core, leaving the hydrophilic portions in contact with the surrounding aqueous phase. The converse arrangement exists if the surrounding environment is hydrophobic. The micelle core may contain the antibacterial lytic protein or mixture of proteins.

As used herein, the term “subject” refers to any organism to which a composition in accordance with the invention may be administered, e.g., for experimental, diagnostic, prophylactic, and/or therapeutic purposes. Typical subjects include any animal, e.g., mammals such as mice, rats, rabbits, non-human primates, and humans. A subject may seek or be in need of treatment, require treatment, be receiving treatment, be receiving treatment in the future, or be a human or animal who is under care by a trained professional for a particular disease or condition.

As used herein, the term “supramolecular structure” refers to a complex of molecules held together by noncovalent bonds, such as hydrogen bonds, Van der Waals forces, electrostatic interactions, hydrophobic effect, and Pi-Pi interactions. Supramolecular structures may include large complexes of molecules that form, e.g., sphere-like structures. Supramolecular structures include, for example, lipid-based supramolecular structures, such as liposomes, lipid nanoparticles, and micelles.

As used herein, the term “targeted intracellular compartment” refers to an endosome, phagosome, lysosome, or cytosol.

The term “targeting moiety,” as used herein, represents a moiety (e.g., a small molecule, e.g., a carbohydrate) that specifically binds or reactively associates or complexes with a receptor or other receptive moiety associated with a given target cell population (e.g., a professional antigen-presenting cell, e.g., macrophage or dendritic cell). Thus, a targeting moiety may be used to target a supramolecular structure described herein to, e.g., a professional antigen-presenting cell (e.g., macrophage or dendritic cell).

“Vesicles” are defined herein as a type of a supramolecular structure in which amphipathic molecules (e.g., lipids) collectively define a volume, e.g., a substantially spherical volume. Amphipathic molecules (e.g., lipids) typically make up at least one shell of a vesicle. In this shell, the amphipathic molecules are arranged in a bilayer with hydrophilic portions of the amphipathic molecules being outwardly directed relative to the plane of the bilayer and the hydrophobic portions of the amphipathic molecules being disposed predominantly within the bilayer. The converse arrangement exists if the surrounding medium is hydrophobic.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are a set of mycobacterial growth curves over five days in the presence of ABIα (FIG. 1A) or absence of ABIα (FIG. 1B).

FIGS. 2A and 2B are bright field images of mycobacterial bacilli rod shaped cells that are shredded by ABIα into subcellular fragments that contain stainable lipids giving rise to red staining bodies that are separated from the genomic DNA. Th experiment was performed at pH 6.2. FIG. 2A shows untreated mycobacterial cells without fragments. Lipids are shown in red, and DNA is shown in green. Green staining DNA indicates dye access afforded by a compromised cell envelope. Intact cells do not readily uptake red dye. FIG. 2B shows ABIα treated cells and fragments. Lipids are shown in red, and DNA is shown in green. Subcellular fragments light up with the lipophilic red dye. The green DNA is spread out as compared to FIG. 2A.

FIGS. 3A and 3B are bright field images similar to FIGS. 2A and 2B except performed at pH 5.3.

FIGS. 4A and 4B are bright field images showing ABIα mechanism by shredding mycobacteria into fragments. FIG. 4A shows untreated mycobacterial cells without fragments. FM4-64 is a red dye that stains lipids, envelop, and lipid bodies. FIG. 4B shows treated mycobacterial cells with fragments. Cytox-Green stains DNA in cells with compromised envelopes.

FIG. 5A is a schematic diagram showing a liposome containing ABIα.

FIG. 5B is a dynamic light scattering analysis showing a mean hydrodynamic diameter of 413 nM and a polydispersity index of 0.19, indicating high homogeneity.

FIG. 6 is a picture of a gel showing ABIα enzymes in a liposome.

FIG. 7 is a table showing activity of ABIα liposome formulations.

FIG. 8 is a picture of a gel showing partially purified ABIα liposomes with improved encapsulation.

FIG. 9 is a schematic diagram showing a proposed mechanism of the ABIα killing mycobacteria inside of macrophages.

FIG. 10 is graph showing ABIα-PPL formulation-8 treatment of J774A.1 mouse macrophages (Mos) uninfected and infected with Mycobacterium abscessus (MOI=10:1). The amount of necrotic cell death for Mos was determined to assess safety and efficacy of ABIα-PPL treatment of mycobacterial infections. The level of necrosis for Mos was quantified as a ratio of the number of cells, stained with SYTOX™ Green Nucleic Acid Stain (Invitrogen™) in the population, stained with Hoechst-33342 (Invitrogen™), counted per FOV (N=2-3 per well) with HCIA platform (BZ-X800; Keyence). Decreases in necrosis were seen in both uninfected (Form8 Mos Only) and infected (Form8 Treated IIMs) Formulation-8 treatment groups compared with the untreated control group (Untreated IIMs). IIMs: Internally Infected Mos; Form8: Formulation 8; HCIA: High Content Imaging Analysis; FOV: Field of View.

FIG. 11 is a graph showing ABIα-PPL formulation-8 treatment of J774A.1 mouse macrophages (Mos) uninfected and infected with Mycobacterium abscessus (MOI=10:1). The amount of necrotic cell death for Mos was determined to assess safety and efficacy of ABIα-PPL treatment of mycobacterial infections. The level of necrosis for Mos was quantified as a ratio of the number of cells, stained with SYTOX™ Green Nucleic Acid Stain (Invitrogen™) in the population, stained with Hoechst-33342 (Invitrogen™), counted per FOV (N=3 per well) with HCIA platform (BZ-X800; Keyence). Decreases in necrosis were observed in both Formulation-8 treated groups (Mos Only and Form8 Treated IIMs), and uninfected Mos treated with empty liposomes (Lipo MosOnly), compared with the untreated control group (Untreated IIMs). Lipo: Empty liposome; IIMs: Internally Infected Mos; Form8: Formulation 8; HCIA: High Content Imaging and Analysis; FOV: Field of View.

FIG. 12 is a graph showing ABIα-PPL treatment of J774A.1 mouse macrophages (Mos) uninfected and infected with Mycobacterium abscessus (MOI=10:1). The level of necrosis and oxidative stress in Mos was quantified to assess safety and efficacy of ABIα-PPL treatment of mycobacterial infections. The level of necrosis for Mos was calculated as a ratio of the number of cells, stained with SYTOX™ Green Nucleic Acid Stain (Invitrogen™) in the population, stained with Hoechst-33342 (Invitrogen™), counted per FOV (N=2-3 per well) with HCIA platform (Keyence). IIMs: Internally Infected Mos; Form8: Formulation 8; Form8 w/Tw: Formulation 8 with Tween-80; ROS: Reactive Oxygen Species HCIA: High Content Imaging Analysis; FOV: Field of View.

FIG. 13 is a graph showing the level of oxidative stress for Mos quantified as a ratio of the number of cells, stained with CellROX™ (Invitrogen™) in the population, stained with Hoechst-33342 (Invitrogen™), counted per FOV (N=2-3 per well) with HCIA platform (BZ-X800; Keyence). Decreases in necrosis were observed in all the treatment groups relative to the untreated control group (Untreated IIMs). Decreases in oxidative stress were seen in two treatment groups (Mos Only and Form8 Treated IIMs**) compared with untreated IIMs. IIMs: Internally Infected Mos; Form8: Formulation 8; Form8 w/Tw: Formulation 8 with Tween-80; ROS: Reactive Oxygen Species HCIA: High Content Imaging Analysis; FOV: Field of View.

FIG. 14 is a table showing various formulations used to assemble payloaded liposomes.

### DETAILED DESCRIPTION

Mycobacteria are actinomycetia (e.g., corynebacteriales or propionibacteriales) that are denoted by a thick cell wall that is rich in mycolic acids. Mycobacteria contain, from outside to inside, a capsule, mycolic acid layer, arabinogalactan (AGL) layer, peptidoglycan (PG), plasma membrane, and cytoplasm. This complex cell envelope contributes to the hardiness of the mycobacteria and is particularly difficult to penetrate and destroy, which is needed for the effective treatment of mycobacterial infections.

The present invention features a composition of matter and methods of use thereof that was rationally designed to degrade the mycobacterial envelope. In general, the compositions described herein feature a liposome that targets the host cell, e.g., macrophage or dendritic cell, and the correct targeted intracellular compartment (endosome, phagosome, lysosome, or cytosol). Meanwhile, the liposome is pre-loaded with a cocktail of antibacterial lytic proteins that are primed to kill the bacterial cell. The liposome directs the payload to the correct cell type and intracellular compartment, while the cocktail of antibacterial lytic proteins degrades the mycobacterial envelope to kill the bacteria.

The compositions include a cocktail containing two or more of Lysin A, Lysin B, isoamylase, and α-amylase. Such a combination of lytic proteins is particularly advantageous in killing a mycobacterial cell. To come up with the protein components we first rationally attacked three layers of the mycobacterial envelope, the capsule, the junction between the mycolic acids and the AGL layer, and the peptidoglycan layer. The components of envelopes at the basic structure levels are observed for many actinomycetia, such as corynebacteriales (e.g., mycobacteria) and propionibacteriales, such as cutibacteria.

To identify the optimal protein components in the composition, we used bioinformatic analysis to identify diverse proteins while screening for critical enzyme characteristics, such as yield, enzymatic activity, purity, thermal stability, and antibacterial activity.

We further identified improved excipients for assembling the liposome with the proteins. These excipients improved encapsulation and reduced precipitation, which are important features for enzymatic activity. These thermostable complexes exhibited robust antimycobacterial effects and can be used to treat infections caused by a variety of mycobacteria and related actinomycetia (e.g., corynebacteriales or propionibacteriales) with similar envelope structures.

### Antibacterial Lytic Proteins

The invention features a supramolecular complex (e.g., liposome) containing one or more of (e.g., one, two, three, or four) of Lysin A, Lysin B, isoamylase, and α-amylase. Moreover, the present invention features the discovery of distinct lytic protein sequences that exhibit robust bactericidal effects. These proteins, shown below in Table 1, exhibit improved expression, thermal stability, and antibacterial effects, e.g., as compared to other orthologs of these proteins.

TABLE 1
Lytic protein sequences

| Protein | SEQ ID NO: | Sequence |
|---|---|---|
| Lysin A | 1 | MEKVLPYDRSIVPQETGYWCGPAATQVVLNSRGLIVPEATLAREI |
| | | GTTVRGTDYVGLIERILDLRVPDARYTSVYIENDPPTADQRETLW |
| | | RNLKRSIDAGYGVVMNWVAPPSNKPRGVKGSVSPRYSGGTTYH |
| | | YVAAMGYDDAGERAVWIADSGFQPQGYWVSFDQCASLIPPKGY |
| | | AYADPTVAPEAPVDADAQAADALLRLMGGSLPFARYQALLPAVR |
| | | QCLDECECDTEARIAMWGAQVGHESVGLKYMSELWGPTAAQR |
| | | GYEGRADLGNTQPGDGYRFRGAGPIQVTGRHNFTVLSQWAHR |
| | | EGLVPTPTYFVDNPDELRGDRYGFVGVVWYWTTQRPMNDAAD |
| | | ARDLVRATQYVNGGQNGIDNRRDRYNGALAMGADLLKIVNGGD |
| | | DFMSALTAAEQREMLDMLRWLAAPGTGELRKKFPSRSELRAVG |
| | | EGLVDTWAGMDLNQDANIHLVAEYVLAKIGDPGAIERLRKLAATT |
| | | ADDRQGSAALARRILDHLDEGGDASEPEPEAPAREVVCESSGG |
| | | PCVLVANGGDGSCALAGNDCVLRKGVSK |
| Lysin A (6X-His) | 2 | MEKVLPYDRSIVPQETGYWCGPAATQVVLNSRGLIVPEATLAREI |
| | | GTTVRGTDYVGLIERILDLRVPDARYTSVYIENDPPTADQRETLW |
| | | RNLKRSIDAGYGVVMNWVAPPSNKPRGVKGSVSPRYSGGTTYH |
| | | YVAAMGYDDAGERAVWIADSGFQPQGYWVSFDQCASLIPPKGY |
| | | AYADPTVAPEAPVDADAQAADALLRLMGGSLPFARYQALLPAVR |
| | | QCLDECECDTEARIAMWGAQVGHESVGLKYMSELWGPTAAQR |
| | | GYEGRADLGNTQPGDGYRFRGAGPIQVTGRHNFTVLSQWAHR |
| | | EGLVPTPTYFVDNPDELRGDRYGFVGVVWYWTTQRPMNDAAD |
| | | ARDLVRATQYVNGGQNGIDNRRDRYNGALAMGADLLKIVNGGD |
| | | DFMSALTAAEQREMLDMLRWLAAPGTGELRKKFPSRSELRAVG |
| | | EGLVDTWAGMDLNQDANIHLVAEYVLAKIGDPGAIERLRKLAATT |
| | | ADDRQGSAALARRILDHLDEGGDASEPEPEAPAREVVCESSGG |
| | | PCVLVANGGDGSCALAGNDCVLRKGVSKHHHHHH |
| Lysin B | 183 | MSKPWLFTVHGTGQPDPLGPGLPADTARDVLDIYRWQPIGNYP |
| | | AAAFPMWPSVEKGVAELILQIELKLDADPYADFAMAGYSQGAIVV |
| | | GQVLKHHILPPTGRLHRFLHRLKKVIFWGNPMRQKGFAHSDEWI |
| | | HPVAAPDTLGILEDRLENLEQYGFEVRDYAHDGDMYASIKEDDL |
| | | HEYEVAIGRIVMKASGFIGGRDSVVAQLIELGQRPITEGIALAGAII |
| | | DALTFFARSRMGDKWPHLYNRYPAVEFLRQI |
| Lysin B (6X-His) | 184 | MSKPWLFTVHGTGQPDPLGPGLPADTARDVLDIYRWQPIGNYP |
| | | AAAFPMWPSVEKGVAELILQIELKLDADPYADFAMAGYSQGAIVV |
| | | GQVLKHHILPPTGRLHRFLHRLKKVIFWGNPMRQKGFAHSDEWI |
| | | HPVAAPDTLGILEDRLENLEQYGFEVRDYAHDGDMYASIKEDDL |
| | | HEYEVAIGRIVMKASGFIGGRDSVVAQLIELGQRPITEGIALAGAII |
| | | DALTFFARSRMGDKWPHLYNRYPAVEFLRQIHHHHHH |
| Isoamylase | 242 | MSAHRTLLLRLSDSGEPVTSCSYGQGVLTLPSLPLPQGKKLGDM |
| | | PVYTVKLAIPAGSPVTRDGLIWTNCPPDFSTQFDREKFYKKIIKTS |
| | | FHEDDHIDLDIYVPGTYCFYLSFKNDKDELETTRKFYFVVLPILSV |
| | | NDKFIPLNSIAMQSVVSKWMGPTIKDWEKVFARVASKKYNMIHFT |
| | | PLQHRGESNSPYSIYDQLEFDPTVFKSEKEVADMVERLRTEHNIL |
| | | SLTDIVFNHTANNSQWLLDHPEAGYNHKTSPHLISAIELDKKLLDF |
| | | SEQMEALGYPVDLKTVDDLIKVMDGIKEHVIGELKLWEFYVVDVK |
| | | QTVSELREKWGNSKSWSDDNIPSKDDSTNLAQFVRDNATEPGF |
| | | GSLGERGSNKINIDKFAAILKKLHSEDYNNGIEELATKILNDINLPF |
| | | YKEYDDDINEVLEQLFNRIKYLRIDDHGPKQGPITKKLPLSEPYFT |
| | | RFKAKDGEEYALANNGWIWDGNPLVDFASSQSKAYLRREVIVW |
| | | GDCVKLRYGKGPSDSPYLWERMSKYVEMNARIFNGFRIDNCHS |
| | | TPLHVGQYFLDVARRVNPNLYWVAELFSGSEAMDCLFVERLGIS |
| | | SLIREAMQAWSEEELSRLVHRHGGRPIGSYKFVPLDDFPYPADV |
| | | KIDEEYCAYNPDDHSVKCVSEIMIPKTLTATPPHALFMDCTHDNE |
| | | TPNQKRTVEDTLPNAALVAFCSSAIGSVYGYDEVFPQLLDLVQEK |
| | | RTYSCAENTGISKVKTLLNNMREEIASEAVDIEDSEMHVHHDGQY |
| | | ITFHRTNAKNGKGWYLVARTKFHSSGDQMLPRIKLSQTKATFKA |
| | | AFSLERTGDAPISDEIIEGIPTKLRELTGFDIGFDENTKETSILLPQD |
| | | FPQGSIVIFETQQLGIDDSLDHFIRSGAIKATEKLSLESINYVLYRA |
| | | EQEEYDYSEGRSGAYDIPDYGKPVYCGLQGWVSILRKIIFYNDLA |
| | | HPLSNNLRNGHWAVDYVVNRLDLYKDKEGVAEVQEWLRSRME |
| | | RIKQLPSYLVPSFFALVVGIMYGCCRLRAMQLMSDNVGKSTVFV |
| | | QSLAMTSIQMVSAMKSTSILPDQNIAAMAAGLPHFSTNYMRCWG |
| | | RDVFISLRGLLLTTGRYEEAKEHILAFAKTLKHGLIPNLLDAGRNP |
| | | RYNARDAAWFFVQAIQDYVTIVPGGVSLLQEKVTRRFPLDDEYIP |
| | | YDDPKAFSYSSTIEEIIYEILNRHAGGIKYREANAGPNLDRVMKDE |
| | | GFNVEVNVDWETGLIHGGSQFNCGTWMDKMGESEKANSVGVP |
| | | GTPRDGAAVEINGLLKSCLRFVLQLSKDGKFKYTEVTKPDGSKIS |
| | | LSSWNDLLQENFERCFYVPKNKEDDNKFEIDATIINRRGIYKDLYR |
| | | SGKPYEDYQFRPNFTIAMVVAPELFTPDYAAGAIELADQVLRGPV |
| | | GMRTLDPSDYNYRPYYNNGEDSDDFATSKGRNYHQGPEWVWC |
| | | YGYFIRAYHYFNFLTNPKCQVEGSAKKLKPSSYLYRKLYSRLLKH |
| | | REWIENSPWAGLAELTNKDGEVCNDSSPTQAWSTGCLLDLFYD |
| | | LWISYEE |
| Isoamylase (10X- | 243 | MSAHRTLLLRLSDSGEPVTSCSYGQGVLTLPSLPLPQGKKLGDM |
| His) | | PVYTVKLAIPAGSPVTRDGLIWTNCPPDFSTQFDREKFYKKIIKTS |
| | | FHEDDHIDLDIYVPGTYCFYLSFKNDKDELETTRKFYFVVLPILSV |
| | | NDKFIPLNSIAMQSVVSKWMGPTIKDWEKVFARVASKKYNMIHFT |
| | | PLQHRGESNSPYSIYDQLEFDPTVFKSEKEVADMVERLRTEHNIL |
| | | SLTDIVFNHTANNSQWLLDHPEAGYNHKTSPHLISAIELDKKLLDF |
| | | SEQMEALGYPVDLKTVDDLIKVMDGIKEHVIGELKLWEFYVVDVK |
| | | QTVSELREKWGNSKSWSDDNIPSKDDSTNLAQFVRDNATEPGF |
| | | GSLGERGSNKINIDKFAAILKKLHSEDYNNGIEELATKILNDINLPF |
| | | YKEYDDDINEVLEQLFNRIKYLRIDDHGPKQGPITKKLPLSEPYFT |
| | | RFKAKDGEEYALANNGWIWDGNPLVDFASSQSKAYLRREVIVW |
| | | GDCVKLRYGKGPSDSPYLWERMSKYVEMNARIFNGFRIDNCHS |
| | | TPLHVGQYFLDVARRVNPNLYVVAELFSGSEAMDCLFVERLGIS |
| | | SLIREAMQAWSEEELSRLVHRHGGRPIGSYKFVPLDDFPYPADV |
| | | KIDEEYCAYNPDDHSVKCVSEIMIPKTLTATPPHALFMDCTHDNE |
| | | TPNQKRTVEDTLPNAALVAFCSSAIGSVYGYDEVFPQLLDLVQEK |
| | | RTYSCAENTGISKVKTLLNNMREEIASEAVDIEDSEMHVHHDGQY |
| | | ITFHRTNAKNGKGWYLVARTKFHSSGDQMLPRIKLSQTKATFKA |
| | | AFSLERTGDAPISDEIIEGIPTKLRELTGFDIGFDENTKETSILLPQD |
| | | FPQGSIVIFETQQLGIDDSLDHFIRSGAIKATEKLSLESINYVLYRA |
| | | EQEEYDYSEGRSGAYDIPDYGKPVYCGLQGWVSILRKIIFYNDLA |
| | | HPLSNNLRNGHWAVDYVVNRLDLYKDKEGVAEVQEWLRSRME |
| | | RIKQLPSYLVPSFFALVVGIMYGCCRLRAMQLMSDNVGKSTVFV |
| | | QSLAMTSIQMVSAMKSTSILPDQNIAAMAAGLPHFSTNYMRCWG |
| | | RDVFISLRGLLLTTGRYEEAKEHILAFAKTLKHGLIPNLLDAGRNP |
| | | RYNARDAAWFFVQAIQDYVTIVPGGVSLLQEKVTRRFPLDDEYIP |
| | | YDDPKAFSYSSTIEEIIYEILNRHAGGIKYREANAGPNLDRVMKDE |
| | | GFNVEVNVDWETGLIHGGSQFNCGTWMDKMGESEKANSVGVP |
| | | GTPRDGAAVEINGLLKSCLRFVLQLSKDGKFKYTEVTKPDGSKIS |
| | | LSSWNDLLQENFERCFYVPKNKEDDNKFEIDATIINRRGIYKDLYR |
| | | SGKPYEDYQFRPNFTIAMVVAPELFTPDYAAGAIELADQVLRGPV |
| | | GMRTLDPSDYNYRPYYNNGEDSDDFATSKGRNYHQGPEWVWC |
| | | YGYFIRAYHYFNFLTNPKCQVEGSAKKLKPSSYLYRKLYSRLLKH |
| | | REWIENSPWAGLAELTNKDGEVCNDSSPTQAWSTGCLLDLFYD |
| | | LWISYEEHHHHHHHHHH |
| α-amylase | 393 | MANLNGTLMQYFEWYMPNDGQHWKRLQNDSAYLAEHGITAVWI |
| | | PPAYKGTSQADVGYGAYDLYDLGEFHQKGTVRTKYGTKGELQS |
| | | AIKSLHSRDINVYGDVVINHKGGADATEDVTAVEVDPADRNRVIS |
| | | GEHLIKAWTHFHFPGRGSTYSDFKWHWYHFDGTDWDESRKLN |
| | | RIYKFQGKAWDWEVSNENGNYDYLMYADIDYDHPDVAAEIKRW |
| | | GTWYANELQLDGFRLDAVKHIKFSFLRDWVNHVREKTGKEMFT |
| | | VAEYWQNDLGALENYLNKTNFNHSVFDVPLHYQFHAASTQGGG |
| | | YDMRKLLNGTVVSKHPLKSVTFVDNHDTQPGQSLESTVQTWFK |
| | | PLAYAFILTRESGYPQVFYGDMYGTKGDSQREIPALKHKIEPILKA |
| | | RKQYAYGAQHDYFDHHDIVGWTREGDSSVANSGLAALITDGPG |
| | | GAKRMYVGRQNAGETWHDITGNRSEPVVINSEGWGEFHVNGG |
| | | SVSIYVQR |
| α-amylase (6X- | 394 | MANLNGTLMQYFEWYMPNDGQHWKRLQNDSAYLAEHGITAVWI |
| His) | | PPAYKGTSQADVGYGAYDLYDLGEFHQKGTVRTKYGTKGELQS |
| | | AIKSLHSRDINVYGDVVINHKGGADATEDVTAVEVDPADRNRVIS |
| | | GEHLIKAWTHFHFPGRGSTYSDFKWHWYHFDGTDWDESRKLN |
| | | RIYKFQGKAWDWEVSNENGNYDYLMYADIDYDHPDVAAEIKRW |
| | | GTWYANELQLDGFRLDAVKHIKFSFLRDWVNHVREKTGKEMFT |
| | | VAEYWQNDLGALENYLNKTNFNHSVFDVPLHYQFHAASTQGGG |
| | | YDMRKLLNGTVVSKHPLKSVTFVDNHDTQPGQSLESTVQTWFK |
| | | PLAYAFILTRESGYPQVFYGDMYGTKGDSQREIPALKHKIEPILKA |
| | | RKQYAYGAQHDYFDHHDIVGWTREGDSSVANSGLAALITDGPG |
| | | GAKRMYVGRQNAGETWHDITGNRSEPVVINSEGWGEFHVNGG |
| | | SVSIYVQRHHHHHH |
| α-amylase | 395 | MATSDDWKSKAIYQLLTDRFGRADDSTSNCSNLSNYCGGTYEGI |
| | | TKHLDYISGMGFDAIWISPIPKNSDGGYHGYWATDFYQLNSNFG |
| | | DESQLKALIQAAHERDMYVMLDVVANHAGPTSNGYSGYTFGDA |
| | | SLYHPKCTIDYNDQTSIEQCWVADELPDIDTENSDNVAILNDIVSG |
| | | WVGNYSFDGIRIDTVKHIRKDFWTGYAEAAGVFATGEVFNGDPA |
| | | YVGPYQKYLPSLINYPMYYALNDVFVSKSKGFSRISEMLGSNRN |
| | | AFEDTSVLTTFVDNHDNPRFLNSQSDKALFKNALTYVLLGEGIPIV |
| | | YYGSEQGFSGGADPANREVLWTTNYDTSSDLYQFIKTVNSVRM |
| | | KSNKAVYMDIYVGDNAYAFKHGDALVVLNNYGSGSTNQVSFSV |
| | | SGKFDSGASLMDIVSNITTTVSSDGTVTFNLKDGLPAIFTSA |
| α-amylase (6X- | 396 | MATSDDWKSKAIYQLLTDRFGRADDSTSNCSNLSNYCGGTYEGI |
| His) | | TKHLDYISGMGFDAIWISPIPKNSDGGYHGYWATDFYQLNSNFG |
| | | DESQLKALIQAAHERDMYVMLDVVANHAGPTSNGYSGYTFGDA |
| | | SLYHPKCTIDYNDQTSIEQCWVADELPDIDTENSDNVAILNDIVSG |
| | | WVGNYSFDGIRIDTVKHIRKDFWTGYAEAAGVFATGEVFNGDPA |
| | | YVGPYQKYLPSLINYPMYYALNDVFVSKSKGFSRISEMLGSNRN |
| | | AFEDTSVLTTFVDNHDNPRFLNSQSDKALFKNALTYVLLGEGIPIV |
| | | YYGSEQGFSGGADPANREVLWTTNYDTSSDLYQFIKTVNSVRM |
| | | KSNKAVYMDIYVGDNAYAFKHGDALVVLNNYGSGSTNQVSFSV |
| | | SGKFDSGASLMDIVSNITTTVSSDGTVTFNLKDGLPAIFTSAHHH |
| | | HHH |
| α-amylase | 397 | ATPDEWRSRSIYQVLTDRFARGDGSPDAPCDTGARKYCGGNYR |
| | | GLISQLDYIQGMGFDSVWISPITKQFEDDWNGAPYHGYWQTDLY |
| | | ALNEHFGTEEDLRALADELHARGMFLMVDVVINHNGWPGDAASI |
| | | DYSQFNPFNSSDYYHPPCEINYDDQTSVEQCWLYTGANALPDLK |
| | | TEDPHVSQVHNDWIADLVSKYSIDGLRIDTTKHVDKPAIGSFNDA |
| | | AGVYAVGEVYHGDPAYTCPYQDWVDGVLNFPVYYPLIDAFKSP |
| | | SGTMWSLVDNINKVFQTCNDPRLLGTFSENHDIPRFASYTQDLA |
| | | LAKNVLAFTILFDGIPIVYAGQEQQYSGDSDPYNREALWLSGFNT |
| | | DAPLYKHIAACNRIRSHAVSNDDAYITTPTDIKYSDDHTLALVKGA |
| | | VTTVLTNAGANAGETTVTVEATGYASGEQVTDVLSCESIAASDG |
| | | GRLSVTLNQGLPRVFFPTDALAGSGLCEN |
| α-amylase (6X- | 398 | ATPDEWRSRSIYQVLTDRFARGDGSPDAPCDTGARKYCGGNYR |
| His | | GLISQLDYIQGMGFDSVWISPITKQFEDDWNGAPYHGYWQTDLY |
| | | ALNEHFGTEEDLRALADELHARGMFLMVDVVINHNGWPGDAASI |
| | | DYSQFNPFNSSDYYHPPCEINYDDQTSVEQCWLYTGANALPDLK |
| | | TEDPHVSQVHNDWIADLVSKYSIDGLRIDTTKHVDKPAIGSFNDA |
| | | AGVYAVGEVYHGDPAYTCPYQDWVDGVLNFPVYYPLIDAFKSP |
| | | SGTMWSLVDNINKVFQTCNDPRLLGTFSENHDIPRFASYTQDLA |
| | | LAKNVLAFTILFDGIPIVYAGQEQQYSGDSDPYNREALWLSGFNT |
| | | DAPLYKHIAACNRIRSHAVSNDDAYITTPTDIKYSDDHTLALVKGA |
| | | VTTVLTNAGANAGETTVTVEATGYASGEQVTDVLSCESIAASDG |
| | | GRLSVTLNQGLPRVFFPTDALAGSGLCENHHHHHH |

The compositions described herein may include a Lysin A that includes an amino acid sequence having at least 85% (e.g., at least 90%, 95%, 97%, 99%, or 100%) sequence identity to SEQ ID NO: 1 or SEQ ID NO: 2. The Lysin A may include or consist of the amino acid sequence of SEQ ID NO: 1 or SEQ ID NO: 2.

The compositions described herein may include a Lysin B that includes an amino acid sequence having at least 85% (e.g., at least 90%, 95%, 97%, 99%, or 100%) sequence identity to SEQ ID NO: 183 or SEQ ID NO: 184. The Lysin B may include or consist of the amino acid sequence of SEQ ID NO: 183 or SEQ ID NO: 184.

The compositions described herein may include an isoamylase that includes an amino acid sequence having at least 85% (e.g., at least 90%, 95%, 97%, 99%, or 100%) sequence identity to SEQ ID NO: 242 or SEQ ID NO: 243. The isoamylase may include or consist of the amino acid sequence of SEQ ID NO: 242 or SEQ ID NO: 243.

The compositions described herein may include an α-amylase that includes an amino acid sequence having at least 85% (e.g., at least 90%, 95%, 97%, 99%, or 100%) sequence identity to any one of SEQ ID NOS: 393-398. The α-amylase may include or consist of the amino acid sequence of any one of SEQ ID NOS: 393-398.

The compositions described herein may include an α-amylase that includes an amino acid sequence having at least 85% (e.g., at least 90%, 95%, 97%, 99%, or 100%) sequence identity to SEQ ID NO: 393 or SEQ ID NO: 394. The α-amylase may include or consist of the amino acid sequence of SEQ ID NO: 393 or SEQ ID NO: 394.

In some embodiments, the composition includes a Lysin A of SEQ ID NO: 1, a Lysin B of SEQ ID NO: 183, an isoamylase of SEQ ID NO: 242, and an α-amylase of SEQ ID NO: 393. In some embodiments, the composition includes a Lysin A of SEQ ID NO: 2, a Lysin B of SEQ ID NO: 184, an isoamylase of SEQ ID NO: 243, and an α-amylase of SEQ ID NO: 394.

In some embodiments, the composition includes a Lysin A of SEQ ID NO: 1, a Lysin B of SEQ ID NO: 183, an isoamylase of SEQ ID NO: 242, and an α-amylase of SEQ ID NO: 395. In some embodiments, the composition includes a Lysin A of SEQ ID NO: 2, a Lysin B of SEQ ID NO: 184, an isoamylase of SEQ ID NO: 243, and an α-amylase of SEQ ID NO: 396.

In some embodiments, the composition includes a Lysin A of SEQ ID NO: 1, a Lysin B of SEQ ID NO: 183, an isoamylase of SEQ ID NO: 242, and an α-amylase of SEQ ID NO: 397. In some embodiments, the composition includes a Lysin A of SEQ ID NO: 2, a Lysin B of SEQ ID NO: 184, an isoamylase of SEQ ID NO: 243, and an α-amylase of SEQ ID NO: 398.

Additional sequences were identified that may be useful in the compositions and methods described herein. In some embodiments, the composition includes a Lysin A that includes an amino acid sequence having at least 85% (e.g., at least 90%, 95%, 97%, 99%, or 100%) sequence identity to of any one of SEQ ID NOS: 1-182 as shown in Table 2 below.

TABLE 2
Lysin A Sequences

| | | SEQ | |
|---|---|---|---|
| | Phage/Host | ID | |
| NCBI ID | Species | NO: | Sequence |
| YP_009954207.1 | Yunkel11 | 1 | MEKVLPYDRSIVPQETGYWCGPAATQVVLNSRGLIVPEAT |
| | | | LAREIGTTVRGTDYVGLIERILDLRVPDARYTSVYIENDPPT |
| | | | ADQRETLWRNLKRSIDAGYGVVMNWVAPPSNKPRGVKG |
| | | | SVSPRYSGGTTYHYVAAMGYDDAGERAVWIADSGFQPQ |
| | | | GYWVSFDQCASLIPPKGYAYADPTVAPEAPVDADAQAAD |
| | | | ALLRLMGGSLPFARYQALLPAVRQCLDECECDTEARIAM |
| | | | WGAQVGHESVGLKYMSELWGPTAAQRGYEGRADLGNT |
| | | | QPGDGYRFRGAGPIQVTGRHNFTVLSQWAHREGLVPTP |
| | | | TYFVDNPDELRGDRYGFVGVVWYWTTQRPMNDAADAR |
| | | | DLVRATQYVNGGQNGIDNRRDRYNGALAMGADLLKIVNG |
| | | | GDDFMSALTAAEQREMLDMLRWLAAPGTGELRKKFPSR |
| | | | SELRAVGEGLVDTWAGMDLNQDANIHLVAEYVLAKIGDP |
| | | | GAIERLRKLAATTADDRQGSAALARRILDHLDEGGDASEP |
| | | | EPEAPAREVVCESSGGPCVLVANGGDGSCALAGNDCVL |
| | | | RKGVSK |
| YP_009954207.1 | Yunkel11 | 2 | MEKVLPYDRSIVPQETGYWCGPAATQVVLNSRGLIVPEAT |
| (6X-His) | | | LAREIGTTVRGTDYVGLIERILDLRVPDARYTSVYIENDPPT |
| | | | ADQRETLWRNLKRSIDAGYGVVMNWVAPPSNKPRGVKG |
| | | | SVSPRYSGGTTYHYVAAMGYDDAGERAVWIADSGFQPQ |
| | | | GYWVSFDQCASLIPPKGYAYADPTVAPEAPVDADAQAAD |
| | | | ALLRLMGGSLPFARYQALLPAVRQCLDECECDTEARIAM |
| | | | WGAQVGHESVGLKYMSELWGPTAAQRGYEGRADLGNT |
| | | | QPGDGYRFRGAGPIQVTGRHNFTVLSQWAHREGLVPTP |
| | | | TYFVDNPDELRGDRYGFVGVVWYWTTQRPMNDAADAR |
| | | | DLVRATQYVNGGQNGIDNRRDRYNGALAMGADLLKIVNG |
| | | | GDDFMSALTAAEQREMLDMLRWLAAPGTGELRKKFPSR |
| | | | SELRAVGEGLVDTWAGMDLNQDANIHLVAEYVLAKIGDP |
| | | | GAIERLRKLAATTADDRQGSAALARRILDHLDEGGDASEP |
| | | | EPEAPAREVVCESSGGPCVLVANGGDGSCALAGNDCVL |
| | | | RKGVSKHHHHHH |
| QGJ96337 | Kahlid | 3 | MAFVSRYGNWYSENGWRMCDANELDRGAVPGTTLVLPI |
| | | | RKGIANLILKAWVAWFHRNVESLNNARGFSDEGAWTPTN |
| | | | DVANSNHLSGTAVDLNWSDHAFRVSYSGFTQAEINKVRE |
| | | | GLRLFEGTIWWGQDWTSPKDPMHFQLNYGEGDARNAAF |
| | | | ATKLQNGYLRIWSGGGGVIEPPAGVDDIYAQEGDSGDRV |
| | | | KRLQQFFNDNFKSYSQLEVDGDFGPATKAVVVEFQKRVG |
| | | | VLADGIVGPITLAAMVKHGFKTESGVKVANLPEDWTDRQV |
| | | | LNDIWEQLRGPDGKGWEQLGKNAKGENLSLVDAVAEVK |
| | | | KRLEAA |
| AYN58177 | KandZ | 4 | MAFIQKQGYWKSENGWRMCDTAELDYTAVPGTSFKLGV |
| | | | RKGSPNIILKALIWRLDKIEPMITSQIGCYTAENSMANSNHN |
| | | | SATAIDYNWNKHPFQKWGTWPNRAAVDKIVDDFRGIIEFG |
| | | | GDWTSPRDEMHFELHFAEGHAGTEALSKDLANGLWGIW |
| | | | KPGAAPAPTPAPGGGNDGILRIGSEGPEVLKMQRGMNSV |
| | | | FKNYRAMPLLEDGIFGAKTKEAVVEFQQRSLIDVDGEVGP |
| | | | QTKAKLAEYGIVLTGATAPTAPPAIVPQKVWPQTASDREL |
| | | | LEYIAAQLGPGDPSWPIKLGNNEKGDPLTVRDRLGLTAKD |
| | | | IEEIKGRLSQ |
| AKF14710 | AlanGrant | 5 | MSFIRSKFAPRALLTVDQWIAIFVAVADELDMPDKRGAVIC |
| | | | AAMCAFQEAGADLDDGRGRQIWIPGNMADPCYAADPDA |
| | | | YPHDSEGNDGQSTGPFQQQMNRPGTTPWGWGGNYGD |
| | | | CTGTRKRMDPWDSTRMFFGWPGSGLRDKGYDASSAQA |
| | | | ANDSIQRVQGSGVPWAYGQHWGLAQAAYARYLGNPIPA |
| | | | PPSPGGGGPAPALAPNPDWRGDPLYLPQLLRAFGVSVTT |
| | | | YTDADGIPWDQRGHGDFGRISWVLWHHTGSVNETDNGI |
| | | | AHHPALGLAANMLIHPDGHVVLTGSGIAWHGGAGIYPGIP |
| | | | EDNINGISIGIECSYGPDRNGQYTLPWPTAQMNAMIAVGG |
| | | | AISWFLGDTLPPSHQIAHKEWAGRDNPLGINKQGKPDPG |
| | | | NLDMTWFRAQIAARATAGPTDQGDDWMSNPDALQMLAD |
| | | | IHRETVTQKSPSRAFVAEDGKQIDTPLGILWNVDGNAWTL |
| | | | VLTEGYWNDVPLAVAVVEDIAANGVRQTSWAGSADKDSD |
| | | | VKFNQWLRDFGQAYCQGLVRRKAQWNAMSAAIIALSAAQ |
| | | | AATPKAAPRKRAAKKATATKADQ |
| YP_009952544 | Amelie | 6 | MIIYRANVEAAKGLVIPRLGNKYVYGGMISPTNLQQGTDC |
| | | | SGIWNDVLGMTVGRFQWGREGEGATTESYRPKTMGGPI |
| | | | PIGGVGPFGTIVVARPQDIPANAVAKLAFHHGPGGGANSH |
| | | | MWGELDGMRIESASSKGLVTAPAAWPIDHSYANAWAYLP |
| | | | GPIVEDGTDVTGIEPQDTLYADVSEWQVPVTDAYANAGY |
| | | | RVLCIRSNDGTYRDKNWATNYAWCKRAVDDGRLKFFIVY |
| | | | FVWRPNWQAAVDTLMSQVGQPHPRMAVMIDVESWGGQ |
| | | | IGGDQSNGINAAYERIAGWLGDRRRVIGYGNVGDLDRLW |
| | | | PRKPEGVRLVVAGYGRLPTYPGLIAHQYTDGQGYGGGLP |
| | | | EGAPPFGNCDMNAANGLTATAFAAALGIDTQEDDSLSALT |
| | | | AAEQRELLDAVRETRFLAKILADKRFVSRSPLRHLGEGAT |
| | | | ETVAGFGLNTDGNLHVLLITELARLGEPGALALLNEVAGA |
| | | | DPVKFPDRQGDRQLAQRILASLGKADAQPEPAPTQPQRK |
| | | | VVCEQGGGSCILVANGGDGSCGLGGDECVIRKGVSA |
| YP_010061237 | Priamo | 7 | MALGAPMENGWPECDLSDCDYATVPGTPLRLPFRKGHP |
| | | | FIILQAFLRDLNEFVEPLMNARGATDEGSWTEDNSVYTSN |
| | | | HKGATAFDYNWSDHPMGNALAGWHGSVLISGEQEPAVR |
| | | | ELLRYYTYRGIQLVWWGNDWYSPKDSMHFQMGYDTVNN |
| | | | PAIVQEFIDKFIRPDGYSTYRRGDSSVPAELTVPLTQLSNG |
| | | | RWTSPSAAWAHLITRESGGNPTIIQQIHDVNSGGNEAEGL |
| | | | FQITPKTWKAHNGTEFAPSARFATPQQQGIVAARIITRNPS |
| | | | GSDWGAGLPGREDAKQLLAGLVPTQTTIDPWEELMALEV |
| | | | ESFSIYANPGEPKIPVHVMIQSLDAHGPHEPYVEKQARQG |
| | | | DRDAIFRVARTAAGKGKYGNAPGPVKQASQVLKELEEAG |
| | | | VLAEYLKGN |
| BBC28650 | PR | 8 | MAVARANVEATKTFIRARLGNPYVYGGALSPTNVKQGTD |
| | | | CSEIWQTTLEMTLGRYVPGRQGEGATTESYRPKSMGGPI |
| | | | PDGGIGPFGTIVTRDRNAVPANAVAKIGLHHGPGGGANS |
| | | | HMWGELDGMGIESRGGGVGVITNPRSMRWDDSYANAFA |
| | | | YLPGPITEDGTPAAPEYVLLGRNYENSGDRVRQLQRALN |
| | | | SYGYGLDEDGEYGPLTEQAVGAFQRSKGLEVDGIAGPVT |
| | | | LAALGLTFGRPVESSPEGLALFRQIMSRTDATNAWLATCL |
| | | | PYYIEAMRAAEINTPLRAAAFASQIGHETSGLKYMAEIQTN |
| | | | GPGWTEDRRIYRGRGAIQLTWSSNYRRFGQWCREQGHV |
| | | | TDPELFVKNPELVEHPQWGFLAASWYWRFGGPKPGQIN |
| | | | SYADKGDILAVSRCVNGWIEGVDPVGWADRRSRYLNCM |
| | | | ALGDRIMNLTTVDPLEELLKMKLNSMSIYRTPGEGPIDAAI |
| | | | MLAALDAHGPHEDYVEKMARRGDEDALFRVARVAMGKG |
| | | | AVTTPAAIKQATDVLLEIAATNRAAVNEALGKV |
| QYW01532 | Pumbaa | 9 | MARRLFRGRAFSENGWPYVDQGSCTWVRIPGAEHVSLQI |
| | | | QNGPPLQVLRAFAADFHAHVEPLRDPDSACWTQDNTVD |
| | | | TSNHPGGTAMDLNWQGADGKTFRYGISEERAYPSPKRE |
| | | | RLRELLDFYEGVVFCGGFWSIQDWMHFQMGAGTYDSKA |
| | | | DRPTEKTLDFIRRKIRPDGFSTFKRGGGGSAAPDAASVLA |
| | | | RAADIPLAKAQEILPTFREGAVLAECTTVPRLAMFIAQTCW |
| | | | ESDRYRATEEYANGPMHEERWIYKGRTWIQLTWRSAYE |
| | | | GFGRWCHARGLVDDPMVFVNNPRSLADLKWAGLGAAYY |
| | | | WTTRRESRKYPTLNEASDARDVLVATQIVNGGTTHLAE |
| | | | RTAIYNRAIALGDELLHILGEEDELANPEIEKMIREVHACLF |
| | | | NRIVSQSIYRFPKNPDGSEHPGNIWQLHELIKNGDGMAHM |
| | | | KYVEDSARGGDLTELDRIAVVAAGRGAVRDPWAVQRAQ |
| | | | RVLADIERTNPEVIRRYLAQKGAA |
| QGJ93182 | TyDawg | 10 | MPLLTPEQIAATIIKVGNEMGVSPKGQKMAIACVKVESNFL |
| | | | MWANAKSKLSQQFPYDRIGNDSLSEGYYQQQPPWWGG |
| | | | ADYEGTRKRMDLVESTRMFFESLKKQRIGTQDYNTDATT |
| | | | PGLWVYMVQRCAPEYKHRYDQEWNAAVALYNKVNAGG |
| | | | SPDAPAPFFPEHNIIDGKGASNRNGQRPRLFVLHTEEGN |
| | | | MVGADLDRWMDEMGDRSYHYAISNDEAWDLVDTDYAS |
| | | | WSVLDANRQTINLVFTPSYAAWTRNDWLAKMGNGIKIAAY |
| | | | LAAQDCRKYGIPPVVRVGNSATGYPSLKTNDGITDHYGIT |
| | | | VGLKIGNHTDVGGGFPWDVFNTYLQAFYAGSYEEDDMFT |
| | | | DSDRALLQRVHFELTNRWESRSIYREPGEGPVDTLVGML |
| | | | LNDDGMEHAELVERLAVLGDFDSIRRVRRTAAGKGAVTD |
| | | | KATVARAKKVMSQIPEDVLEEYMKEADK |
| ATW60139 | Ph8s | 11 | MTVIITRQRAQEVHDRARARKGLPYAYGGAFTNDPRRST |
| | | | DCSGLVMQTAAWYMGRTDWVGNRYGSTESFRLDHKIVY |
| | | | DLGFKRLPPGGVAALGFTPVMLVGLQHGGGGMYSHTAC |
| | | | TLMTMDIPGGPVKVSKRGVDWESHGNRNGVGVDLYDGA |
| | | | RAWNDPLFHDFWYLDAKLEDAPAPEDDAVEILSQATGLS |
| | | | TERALEILPAVRDGLIASECVNANRIAMWLAQVGHESASF |
| | | | KYTEEIAKNGRYAPYIGRTWIQITWDYNYRAFSQWCFDRG |
| | | | LVSTPDYFVRNYTDLALLKWAGLGAAWYWTVARTDINAL |
| | | | SDRRDLETVTLRINGGHNGIADRRERYDRASALGDRLLALI |
| | | | SDAQPIDPFEEEMMREVESFSIYATPGEPKIPLFVMLQSLD |
| | | | AHGPHEPYVEEQARQGDRDAISRVIRTAAGKGKYGTASG |
| | | | PVNQASRVLAELEDSGVLSNYLKGN |
| YP_010056835 | Phabba | 12 | MTTPAVPTGNVSPPGPTPGTGYSQTSPDQDMQADTYYL |
| | | | TDVGGKEPPPMETLVYTPRVRIIIARDNKQYDVSEDVVAV |
| | | | SINRVENSVSSAAFRLINKPVDQYDENERQGRYTPLFAPM |
| | | | DRVAIFMKRTEWVQVFSGYLDSVALAQLYPGTVDFKASC |
| | | | TLKKLLHTWWDPGLPDSAKIFDQFANDVNEADGQESQLD |
| | | | KGLGSTLRRLLIEVGNMDSSQIHIQRFPMGYLNFMRQQLE |
| | | | KLKTSDATVQEFRRMLLGDDTSGGVGAAAGRQLGVTKG |
| | | | AYNLDQAGRKLEVIRAVDEMGMGVDVRSLGLGQGTNTAA |
| | | | QGGADYQDQEAWKAFAELGRNFQDAALKSDAAVHCFMT |
| | | | IAAESSWVMYANTGVPDSLNFPHEALGHDHDSVGLYQQR |
| | | | QSWGTIDQRMNARESTGMFLNELRKHEWRNMPRPAAC |
| | | | QAVQRSAFSDGSNYAVHEAVAIEEVRALRSASGTGQGTG |
| | | | AQSGGATGIPSIGTAPPPVTAGVPGSTGIPGIPTANGQVS |
| | | | SQGVNQTLNRPQYDTAEAIQFGMLQLGKPYAWGAKGPD |
| | | | SYDCSGFTRACYRYIGMNIGDDTYSQVASGTQITMAQAK |
| | | | PGDLIFPAGHGHVVMYLGGNQVIHSPQTGDVVKVSPLWF |
| | | | TPATVITYPGTGYSGPSPLPYDPVRGAQASNGGSDGGVV |
| | | | PGTVAQGTNGGTFQTGSSEPIARNLFSYMFEPGRFQSGI |
| | | | SALFGNDEGTTEKAFINDEPLIQSVMSIARAGLRNVQSMP |
| | | | DGSFAAYYPDYFGLDGKDAILDLEDIEMKNVNINWNDDAL |
| | | | ATHVYVAGTANPTGGSMGLPGWLMTKGVATVENEWLFR |
| | | | RMTSAAPSVKGAEMRNGKEIMKRFGMRPLVQDMPNLFN |
| | | | GEMEFLMALQIFMTKWAEQYSTNVEFTFMPELYPGMRIN |
| | | | LVGHNLQVYVAAVTHSGDWENGFTTSATIMAPSNPNIHRI |
| | | | ASQVDKDLGFGTHWDQRDIDGKPMPDDYKWMY |
| YP_010056919 | Phabba | 13 | MALITENGWPQVPRSLCDNLLVPGTTKVRPELRKDDVTIIL |
| | | | VAWACWFDRNVRNIEPPDGHRNWWAWSATNDVWNSN |
| | | | HLSGTALDLCADELPWQRKTMPQNQVEITNRGIALFEGTV |
| | | | FWGRNWSRTDEMHFQVGLPPTSPKIREFADRLRNGYLNI |
| | | | FGPADPNAFPLPLGYYYGPLDGPVESISGEYESDSQAAK |
| | | | DGLGRWQAALGLPVTKKWNDGLTPRAAYVLQRSKGWLP |
| | | | NPLFGYGGVYAGEWDAVIREGWRLPANTNLHDVEIPDFE |
| | | | YPLTKWGDYSQYQAATVDDSYPYEVISFRASIANQIDTKW |
| | | | LANMKAAQTLVRQGKLKKIIAYHFWVPGADNWGTFRQAIE |
| | | | LAGGVTKELCFMIDVEDGGTKWNIRGDQTAGVKSFIASG |
| | | | QTYFQNPQAASIYWNPTANPDLLVGINDRELRGVKLIVPR |
| | | | YAGPDKAPWTPDGVQWFGHQYSDRENTPPFGPTDINQA |
| | | | KMPLSIFLAAWGANGGPTPDPDDGAGVPSVPVPPVDTDS |
| | | | GVVIPDWDDERVLVAIGAQFDA |
| YP_010064286 | Phaded | 14 | MARRLFRGRQFSENGWPYVDQGSCTWDEVVPGVWLQI |
| | | | QNGIPFTVLRAFARDFNEHVEKLRDADSACWTQDNSVDT |
| | | | SNHPGGTGADFNWNGEDGRTFRYGITEARAYPGDKARR |
| | | | LRELLDFYEDNVFCGGFWDIRDWMHFQMGYGTYDSKAD |
| | | | RPTQKVTDFVARKIRPDGWSTFKRGGGGAPAPAADAAAI |
| | | | LARATGVTLDKAREILPTLRDGLILSECTTVPRIAMWLAQC |
| | | | GHESAHFNATEEYQNGPMDQERWIYKGRTWIQLTWRSA |
| | | | YEGFGRWCHARGLVSDPTVFVNNPRSLADLKWAGLGAA |
| | | | YYWTTTVRSTRKYPTLNQASDARDVLVATQIINGGTNGLE |
| | | | DRQNRYNRAIALGDELLQIIGEEDFLSALTPAEQRELLDLL |
| | | | RWGFAPEYGEFRKLFQNEDMYREDNVRRRTAVGAALDS |
| | | | RTFSWEDRVEKSAERGELWAIDLVVRAARGTLPGVIRPG |
| | | | GNTPDPFLVNHARQVLADVERINPEALKKYLALEGGSR |
| YP_008409872 | Phelemich | 15 | MTERVLPFDRKIIRQETGYWCGPASTQVALSARGKYVDE |
| | | | ATLARECKTTVNGTDNVGQIERVLDVRLPEGKYTSMYPG |
| | | | GTTIGTPARPAAERKTRFWWDIVRSIDNGFAVILNWVVPP |
| | | | ARKPIKAVKGSTNPSYGSGTTYHYVTAVGWSDEGNGGR |
| | | | PAVLIADSGFSPNVYWVDLDTAFALIHTDLWKGYAFADLP |
| | | | LIAPPPPGVEVPPGIPVKIGDPPIAVTPPAPVPPTQPPTVVK |
| | | | ITDPFTGAIWSPNRYSPRGLGTPGWIAVHTQEGGRTARDL |
| | | | ALFLANPANEVSYHSVNDDVEVLKVVAEGDASWSASNAN |
| | | | KYAFHHCFAGSYAGWSRDKWLSPDASDGKNEDVQLTKG |
| | | | AHVVAWWCDKYGIPAEWIGGRAQPPWGARGILGHVDLG |
| | | | QWGGGHFDPGGNFPVNEFIRRVVKFLTGEDQPPLVPLPP |
| | | | VVVPGTNPDAYSDWMLVRGDPRNDVDRVMRVQRRLKN |
| | | | AYKGYAGHLAVDGDFGPATQAAVREFQRRSNLVADGIVG |
| | | | PMTAAALRP |
| YP_009953019 | Curiosium | 16 | MTEKVLPYDRSIVPQETGYWCGPAATQIVLNTRGLIVPEA |
| | | | TLAREIGTTVRGTDYVGLIERILDLRVPDARYTSVYIENDPP |
| | | | SAAQRETLWRNLKRSIDAGYGVVMNWVAPPSNYPRGVK |
| | | | NSASPRYGGGTVYHYVAAMGYDDDPAARAVWIADSGFQ |
| | | | PQGYWISFDQCASLIPPKGYAYADPTVAPEAPIDADAQAA |
| | | | DALLRLMGGSLPFARYQALLPAVRQCLDECDCDTEPRIA |
| | | | MWGAQVGHESVGLKYMSELWGPTAAQQGYEGRADLGN |
| | | | TQPGDGYRFRGAGPIQVTGRRNFTVLSQWAHGKGLVPT |
| | | | PTYFVDNPDELRGDRYGFVGVVWYWTTQRPMNDAADAR |
| | | | DLVRATQYVNGGQNGIDDRRDRYNGALAMGADLIKILNG |
| | | | GDDFMGALTAAEQREVLDLLRWIAAPGTGELRKRFPSRS |
| | | | PLRHLGEGLVDTAVGVGLNDDANDHVVLVKDLAEIGDPD |
| | | | ALALLHEVAGPENTPERYPDRQRDKVLAQRILDSLSKAPQ |
| | | | VGDTPIVVNTPARRVVCESSGGPCVLVANGGDGSCALAG |
| | | | NECVLRQGATK |
| NP_046825 | D29 | 17 | MTLIVTRDHAQWVHDMCRARAGNRYGYGGAFTLNPRDT |
| | | | TDCSGLVLQTAAWYGGRKDWIGNRYGSTESFRLDHKIVY |
| | | | DLGFRRLPPGGVAALGFTPVMLVGLQHGGGGRYSHTAC |
| | | | TLMTMDIPGGPVKVSQRGVDWESRGEVNGVGVFLYDGA |
| | | | RAWNDPLFHDFWYLDAKLEDGPTQSVDAAEILARATGLA |
| | | | YNRAVALLPAVRDGLIQADCTNPNRIAMWLAQIGHESDDF |
| | | | KATAEYASGDAYDTRTDLGNTPEVDGDGRLYKGRSWIMI |
| | | | TGKDNYRDFSRWAHGRGLVPTPDYFVVHPLELSELRWA |
| | | | GIGAAWYWTVERPDINALSDRRDLETVTRRINGGLTNLDD |
| | | | RRRRYNLALAVGDQLLTLIGDDDELADPTIQRFIREIHGALF |
| | | | NTVVTQSPYGDPQNPDGSEPRSNLWQLHELIKNGDGMG |
| | | | HARYVEESARAGDLRELERWRAAKGLGRDRSPEFIARA |
| | | | RNVLAQIEAANPEYLQAYIARNGAL |
| YP_009205384 | Larenn | 18 | MALGGRMENGWPECDLSDCDFATVPGTPLRLPFRKGHP |
| | | | FIILQAFLRDLNEFIEPLMNSRGATDEGSWTDNNSVYTSN |
| | | | HKGASAFDYNWDDHPMGPAAPDPRAGWNGSVLIDGDQT |
| | | | PAVRELLSFYTYKGIQLVWWGNDWDSPKDSMHFQMGYN |
| | | | TVNNPAIVQEFIDKYIRPDGFSTFRRGGNAPQAPVVDAAT |
| | | | VLAKAVGIGYEKAKTILPAVRAGLIQSECKSSPRIAMWLAQ |
| | | | VGHESDSFEATEEYADGNEAEERWKYKGRTWIQLTWLS |
| | | | AYQGFGRWCFDRGLVSDPNCFVNDPRSLAELRWAGLGA |
| | | | AYYWTTTVRSTRKYPTLNQASDARDVLVATQIVNGGTNG |
| | | | LDKRQARYNRAIALGDELLQILGGDNDDMAQVPQDQWS |
| | | | WVLKAVSELHGAMFNRIVSQSPYRYPKNPDGSEPPGNV |
| | | | WQLHELIKNGDGMAHMAYVENAARGGDLTELGRVVRAA |
| | | | RGQGAETAPWFVQRAQRVIAEIEKTNPDLLKRYIAERGVA |
| QXO12831 | Latretium | 19 | MTTKDQVAQITIAEAKARGYTRSECLAVMSTFYQESGWN |
| | | | DTIWDPTHTTYGIAQQDGSYPHRFDGAAAQIKGFFDKLDV |
| | | | WRAKPGASTDIWLNICWMQQAPNWPSADYWYANGRRA |
| | | | YLTEIKSRIATVTPYLDKYWPTTGGTDVPETRPPYNEFPIW |
| | | | SENHYNTKRTVNDIDAFLLHTSEGFVGRDDAAEALSLWY |
| | | | QPRSRQVAYHYAVSQASDGGVTVVDNVDTDYASWSALS |
| | | | ANGRSINLCFAGTRAAWSRNEWLGKFGNAIDVAAYLAVQ |
| | | | DCKKYNIPTKVIAPPYTGRLPGITDHRYVTQILKDGTHTDV |
| | | | GDGFPWDYFTERVNHWANGGKTEPEPPKVKRFPDDWT |
| | | | DREILVEILRQLRGYNLTGWPQLGGKTLVDAVAELLGH |
| YP_010051698 | Lemuria | 20 | MATRFMPMKAGTYSIASGFGPRWGTQHRGLDFAAADGT |
| | | | PIYAAQAGTVAHIGSAQGFGQWIVVDHPAEDGAGTTVYG |
| | | | HMWNAFATGLKLGSHVKAGQLIGFVGSNGQSTGPHLHFE |
| | | | VHPTVWAAGSQIDPAPWLRGALDPGGDPPPAIVKPALPP |
| | | | APAPPPGGNTVGDPVWLPEALRPAVSNLVEYPGWRNRG |
| | | | HGDFKDIRGVMVHHTGGPASARSIADGRPDLAGPLSQLHI |
| | | | ARDGTVTIIAAGVAWHAGSGSYPWLPANMGNWHLIGIEC |
| | | | EWPYRGGIGERNAHEEPWRREQILAIRNTCAALLQWLRF |
| | | | GVDRLIGHKDYAGRAQGKWDPGNMSMPWLRGEVEKDM |
| | | | SGFAFPGEDGPRPIIVDSGLPPVPLPPAPAPAVAGVLLHR |
| | | | GMNGPAVRRLQEVLRRWYSKLAVDGDFGPATEAAVRDF |
| | | | QRLRPPLDADGVVGPATAQRLGL |
| AXF51529 | Constella | 21 | MAVTRANVEATKNFIRARVGNPYVYGGALSPTNVRQGTD |
| | | | CSEVWQTVLEMVHGRYNPGRQSEGATTESYRYIPVGGV |
| | | | GPFGTIRVNHWRDIPANAAAKLAFHHGPGGGANSHMWG |
| | | | ELDGMLIESAGSKGLVTNGRAMTIDNSYATAWAYLPGPIL |
| | | | EDGSPIPEDPNAVTWGIDISNHQGEMDLNRVKAEGFDFIW |
| | | | CKVSEGANYRDPFWPGNRDRARAAGLILAGYHYVRTGD |
| | | | PAAQAKTFVEHLGDKSIPAMLDFEDGSGNIEQFWAVKAEI |
| | | | EKLGVQVRLSYIPDWYHERIGKPDLSKVPGLIASEYVSGT |
| | | | GYASVLYPGNNSNFWKAYGGRTPDVLQFTDRALVAGKS |
| | | | VDANAFRGTPDKLRRLLGAGGDDFLSALSDAEQRLLFDR |
| | | | TNQLWGAVFNDIPSTSKYKAAGEGAIWKSKDLWRNIDGM |
| | | | MHETLVERQAMMGNPEALALVKREADKGDKWAACVYKY |
| | | | CTEEA |
| YP_654941 | Cooper | 22 | MSFIRSKFAPRALLTVDQWIAIFAAVADELDMPDKRGAVIC |
| | | | AAMCAFQEAGADLEDGNGRQIWIPGNMADPCYADDPDA |
| | | | YPHDSEGDDGQSTGPFQQQMNEPGKAPWGWGGNYGD |
| | | | CAGTRKRMDPWDSTRMFFGWPGSGLKAKGYDASSAQA |
| | | | ANDSIQRVQGSGVPWAYAQWWGLANAAYDRYLGNPIPA |
| | | | PPSPGGGSGPAPALVPNPAWRGDPLFLPQVLRAFGVNV |
| | | | STYTDADGIRWDQRGHGDFGKISWVLWHHTGSVNETDE |
| | | | GIAHHPALGLAANMLIHPDGHVVLTGSGIAWHGGIGIYPGI |
| | | | PEDGINQVSIGIECSYGPDENGQYTLPWPTAQMNAMIAVG |
| | | | AAISWFLGDTLPVDHQISHKAWAGADNPLGINKQGKPDP |
| | | | GNLDMNWFRAQIAARTALGPTTGGDDWMADPTALELLR |
| | | | DIHRETVTQKSPSRSFMAEDGKLIDSPLGIEWNTDGNAWT |
| | | | LVLTEGYWNDVPLAIAVVEDIAANGVRQTSWAGSADKDS |
| | | | AVKFNQWLRDFGQAYCQGLVRRKAQWNALVAAVASLSA |
| | | | AQAAGATTAVKAAPRKRAAKKAPAPKDAE |
| ARW57121 | Zenon | 23 | MITENGWPDCGPNDLDLKPIPGTDVVIPLQKGQPSLILKAF |
| | | | AADLNWYVESVYNSRGGTDEGGWTGTNKVPTSNHLGGT |
| | | | AFDYNWSDHPLGYAAPDPRAGWNGSSIIKGDQTPYVREL |
| | | | LDYYEGMVYWGNDWRSPKDSMHFQMGYNTYGPAGSTT |
| | | | WKRVEDFIARKIDITTGFSRFKEHKGTTGVNFTALLSEAML |
| | | | DVSGVDYEYYVPLVARTLRDCECTTEARIAMWCTQVGHE |
| | | | SLSLKYMEEIADGSKYEGRTDLGNTQPGDGKRFKGRGPI |
| | | | QVTGRYNYTKFSEWAHSKGIVSTPDYFVMNPSELSKPEY |
| | | | GFHAVTWYWTVQRPMNQAADARNLEQATKYVNGGLNGL |
| | | | PDRKMRYDRTTAMGPRLLNLLLGEEDWMSDPTIVKMIKEI |
| | | | HGCLFNQIGSQSRYRADGEGDIWKLHELIKNDDGMVHEA |
| | | | HIERLAVLGDPSSIALVARNSERGDKLAQAVLIRIEDKYLTE |
| | | | TQQHLKNTIIAQRFPEG |
| YP_009013589 | Dori | 24 | MPILRANVDYFWSIARPRNRKPYGYGGVWVKNDLNRTTD |
| | | | CSGIVTHALDAVINGPNMTWSRHGISTESYRYVGGPGSR |
| | | | GPFGTIRVARPQDIPADAAVKIGLMHGPGGGANSHMACTI |
| | | | EGVAIESRGGTVASGGGQWVGGNGRHYNNPLFHDWFYL |
| | | | PGPIVGSGASTPAQPQAPGAVYLGRDCSRYECTGERVKA |
| | | | LQARLNRDYPAYSDLDEDGEFGPLTEAVVKQFQLRSALT |
| | | | ADGIAGPATLAALGLSFQAQEVPPVTAPKPVIVGPADDQL |
| | | | TMRWNALGGQTLVEAVAQIRDKVCGTEDRSKPGVVTK |
| QZD96992 | Drake 94 | 25 | MALGGRMENGWPECDLSDCDYATVPGTPLRLPFRKGHP |
| | | | FIILQAFLRDLNEFIEPLMNARGATDEGSWTDNNSVYTSN |
| | | | HKGATAFDYNWSDHPMGNANAGWNGSVLIAGDQVPAVR |
| | | | ELLRFYTYRGIQLVWWGNDWNSPKDSMHFQMGYNTVNN |
| | | | PQIVQEFIDKFIRPDGFSTFRRGGAAPSAPAVDAATILARA |
| | | | TGIAYAKAQEILPTLRDGLILADCKSVPRIAMFVAQTCWES |
| | | | DHYNATEEYANGPLNEERWIYKGRTWIQLTWKSAYAGFG |
| | | | QWCFERGLVNDPNIFVNHPRMLAETRWAGLGAAYYWLT |
| | | | TRRPTRKYPTLNQASDARDVLVATQIINGGTTHLAERQAL |
| | | | YNRAIALGDDLLLILGGDEDEMAGWDQTKVDRAMVLLENI |
| | | | AGVRRKSRSPLRWPYEKEIDTAIGFAWTADANVHVQLVE |
| | | | KLAVIYGDPTSIALLTAVANTDEPGREGDRELARRILARCS |
| | | | EDAVQEAHKRINEWLEAEKAHKVGV |
| QGJ88290 | DreamTeam1 | 26 | MVTRANVEATKEFIRARLGNPYVYGGALSNNVRQGTDCS |
| | | | EVWQTVLEMAHGRWVPGRQAEGATTESYRGIKPGQVGP |
| | | | FGTIAVAHWRDIPANAAARIAFHHGPGGGANSHMWGELD |
| | | | GMRIESGGSKGLVTGDRALAVESTYATNWAYLPGPIGGT |
| | | | ASLGIGSTGPAVITLQAKLNTRGAGLEVDGEFGPLTAAAVT |
| | | | RAQQELHVVGDPPGIAGEATLRALGILNDNGAIPANPDTL |
| | | | AAEYLAAATGLSVARAQQILPAARDGLLRSDATNVNRIAM |
| | | | WLAQIGHESAGFNATEEYEKGDGGATERWKYLGRTWIQL |
| | | | TWLSNYLGFSKWCFERGLVPTPTYFGDNPRELADLKWA |
| | | | GLGAAYYWTVARPDINALSDRRDLDTVTRRINGGTNGIED |
| | | | RRNRYNRALAVGDNLLRIIANSPEPQDEWEALMADQTRY |
| | | | SSRAWFRSDDVANLTPLDLLRNVDAMKYDERVIQAAFRG |
| | | | QVWAINAIATLAKGEGPEGKNPAAVLEAQDIIRQLQAALAA |
| | | | QNGASK |
| QJD51802 | VC3 | 27 | MTLIVTREHAQWVHDMCRARAGNRYGYGGAFTLNPRDT |
| | | | TDCSGLVLQTAAWYVGRKDWIGNRYGSTESFRLDHKIVY |
| | | | DLGFKRLPPGGVAALGFTPVMLVGLQHGGGGVYSHTACT |
| | | | LMTMDIPGGPVKVSQRGVDWESRGEVNGVGVFLYDGAR |
| | | | AWNDPLFHDFWYLDAKLEDGPTQSVDAAEILARATGLAY |
| | | | NRAVALLPAVRDGLIQADCTNPNRIAMWLAQIGHESDDFR |
| | | | ATAEYASGDAYDTRTDLGNTPEVDGDGRLYKGRSWIMIT |
| | | | GKDNYRDFSRWAHGKGLVPTPDYFVVHPLELSELRWAGI |
| | | | GAAWYWTVERPDINALSDRRDLETVTRRINGGLTNLDDR |
| | | | RRRYSLALAVGDQLLTLIGDDDELADPTIQRFIREIHGALFN |
| | | | TVVTQSPYGDPKNPDGSEPPSNLWQLHELIKNGDGMGH |
| | | | ARYVEDSARAGDLRELARVVRAAKGLGRDRSPEFIARAR |
| | | | NVLAQIEAANPEYLEAYIAKNGAL |
| YP_009950928 | Rando14 | 28 | MTEKVLPYDRSIVPQETGYWCGPAATQIVLNSRGLIVPEA |
| | | | TLAREIGTTVRGTDYVGLIERILDMRVPDARYTSVYIENDP |
| | | | PRTEQREALWRNLKRSIDAGFGVVMNWVAPPSNYPRGV |
| | | | KGSVSPRYSGGTVYHYVAAMGYDDAAPGVGRAVWIADS |
| | | | GFQPQGYWISFDQCASLIPPKGYAYADVDAPGGPAAPVD |
| | | | ADAQAADVLMRLMGGSMPFGYYQEALPAVRQCLVECGC |
| | | | DSELRIAMWGAQVGHESVGLRYMSELWGPTAAQAGYEG |
| | | | RVDDLGNTQPGDGYRFRGAGPIQVTGRRNFTVLSQWAH |
| | | | SKGLVPTPTFFVDNPDELRGLRYGFVGVTWYWTTQRPM |
| | | | NDFADRRDIEGASIAVNGRGANGRANGIDDRINRYNGALA |
| | | | MGADLLKITDGGDDFMSALNAAEQREMLELLRWIAAPGT |
| | | | GELRKKFPSRSELRAIGEGAVDTWAGMDLNQDANIHLVA |
| | | | EYVLAKIGDPGAIERLRKIASTTAADRQGSAALARRILDHV |
| | | | DEPHETPPDSEPAAPVRKVACAQSGGGCVLVANGGDGS |
| | | | CALGGDDCVLRKGVTA |
| YP_009950179 | Aminay | 29 | MANRIVYGRSNSENGWPMVDAGSCQWVTVPGTNVSLQI |
| | | | REGQPAKILGAFVADINAYVENVTDADSGCWTPTNSVASS |
| | | | NHLSGTACDVSWNRHPFHVRNTYNAAQRATIRELLDWYE |
| | | | DTVFWGGDWNSPIDEMHYQMGYDTYGSQNVARVQDFIN |
| | | | RKIRPDGFSTFRRGGSTAPAPAPQPAVDQVQVLANATGL |
| | | | SRGRAAEILPAVVDGLAASSCKNVNRIAMWLAQIGHESDN |
| | | | FNATEEYDKGDGGRTERWKYLGRTWIQLTWASNYAGFS |
| | | | QWCYGRGLVNSPTYFVDRPAELAELKWAGLGAAYYWTV |
| | | | ARPQINSLCDARDLLAVTKAINGGTNGLPDRQNRYNRALA |
| | | | VGDALLALATSTSTPTQEDDMAQVPQEQWDRVYRELTQ |
| | | | RLPSRSPLRHLGEGPVDTLAGFVLNADGSEHVEIVRLLAG |
| | | | YGHPPTLALLREVAGADPVRYPDRQEDRKLAQAILAEVTA |
| | | | TPAAPAATVASVAPTEPQIVYLPAPTPEPVAAPPAPTPEPV |
| | | | AAPPAPAAGGSTGQIIGQAYDALEALQLSGVLEAAERAPL |
| | | | SALITVLQTKTQGASA |
| ALF01176 | Annihilator | 30 | MDDRDMADRFFPMRDGTYTLSSGFGARWGTQHRGLDF |
| | | | AAKDGTPIYAAQAGTVAYIGPAQGFGQWIVIDHPAADGAG |
| | | | TTVYGHMWNAFATGLKAGDRVQAGQLIAYVGANGQSTG |
| | | | PHLHFEVHPTVWRQGSQIDPLPWLHGSRNPGDAPAPAP |
| | | | ADPPASAPLGGQRMQDPFTGEVWSPNRSVRQKPAPRWI |
| | | | AIHTQEGGRTARDLCEGWLAKRESQVSYHVAVDDREILK |
| | | | VVAESDRPWAAANANDYAFHVVAAGSYAGWSRGKWLET |
| | | | DASDGKNEDVELTNLAKVCAWWCQKYGIPAEWIGGRGV |
| | | | PWGLDGICGHEDFGAWGGGHHDPGPGFPADELIRRVRA |
| | | | LLGGSTPEPLPPAPPVALPGTNPDQYAGVLLYRGRPGQD |
| | | | PRQVRVLQTRLKRAYSKLDVDGIFGPHTEACVRDYQHLH |
| | | | PPLVADGIVGPATAAALGLVF |
| YP_010062045 | Anthony | 31 | MTAVVTRQRAQQVHDMARARAGLPYAYGGAFTNDPKRS |
| | | | TDCSGLVMQTAAWYMGRTDWVGNRYGSTESFRLDYKIV |
| | | | YDLGFKRLPPGGIAALGFTPVMLVGLQHGGGGMYSHTAC |
| | | | TLMTMDYPGGPVKVSKRGVDWESHGNRNGVGVDLYDG |
| | | | ARAWNDPLFHDFWYLDAKLEDAPTSGDEAAATLSEATGL |
| | | | SLQRASEILPAVRDGLVASDCTNANRIAMWLAQVGHESV |
| | | | SFNYTEEIAKNGRYAPYIGRTWIQITWDYNYRAFSEWCFE |
| | | | RGLVTSPDYFVRNYTDLALLKWAGLGAAWYWTQARGTQI |
| | | | NDASDRRDLYTVTQLINGGQNGAADRKARYHRASALGDR |
| | | | LLSLIADAPTTDTFEELLMMEVESFSIYADPGEPKIPLYVML |
| | | | QSLDAHGPHEPYVEAQARIGHADSIRRVVRTAAGKGKYG |
| | | | TAPGPVNQASRVLAELEASGVLAAYLKGTA |
| YP_010051567 | Antsirabe | 32 | MADRYLPMREGTYQIASGFGPRWGTVHRGLDFAAKDGT |
| | | | PIYAAQAGTVVHIGAAQGFGQWIVVDHPAEDGAGTTVYG |
| | | | HMWNAFATGLKQGARVEAGQLIGYVGSNGQSTGPHLHF |
| | | | EVHPTVWAAGSQLDPAPWLRGARNPGDPTAPRPAPAPA |
| | | | PAPAPPPGGNTVGDPIWLPDALRPAVSNLVEYPGWRNR |
| | | | GHGDFKDIRGVMVHHTGGPASARSIADGRPDLRGPLSQL |
| | | | HIARDGTVTIVAAGVAWHAGAGSYPWLPTNMGNWHLIGI |
| | | | ECEWPYRGSGISERNAGDEPWRREQIIAIRNTCAALLSW |
| | | | MRFGVDRLIGHKDYAGRAQGKWDPGNMSMPWLRGEVE |
| | | | KDMSGFAFPGEDGPRPIIVDTGLPPVPLPPAPAPAYAGVLI |
| | | | HRGMTGPAVRRLQETLRRWYSKLVVDGDFGPATEAAVR |
| | | | DFQRLRPPLAADGVVGPATATKMGLVL |
| YP_009009240 | Jolie1 | 33 | MGMTLENGWPECDLSDTERLTIPGTALSLPIRKGQPHAIL |
| | | | QAFFRDVNEFIEPVMNARGLSDEGSWTENNSVYTSNHKG |
| | | | ATAVDWNWSDHPVQIRDAGWDGSVLINGSQVPAMRELL |
| | | | AWYEGMVFWGNDWNSFIDSMHFQMGYNTFGAQNFDRV |
| | | | HSFIQRKIRADGFSTYRRGGTPRGGGFAEVPAAPLHPIKP |
| | | | TSGLTPEVLWRIAGGAASKLSVGHFERWFDELVECQAAC |
| | | | GVLGNIDRSAMWYSQVFPESGNLVYTEEIASGAAYEGRC |
| | | | EGLGNCQPGDGVRFKGRSFMQVTGRSNYTKMSGWAHG |
| | | | KGYVPTPDYFVVHPDQLDDERFAFLGVTWYWTTQRPMN |
| | | | DAADARNLELATRYVNGGLTNLEGRRAVYNRALAENANL |
| | | | LLTNPVEPWEELMATAVPSLSIYANPGEPDVPLAVMLAAL |
| | | | DAHGPHEPYVERQAIEFGDADSIRRIARTANGQGKVKTPA |
| | | | AIKQATDAFRLIPAEFVRAAIPA |
| YP_009009684 | Jolie2 | 34 | MADRCMPLADGTYRLGSLFGNRKGGFHRGIDFEADDGT |
| | | | PIYASQAGTVAHIGSASGFGQWIVIDHPAEDGAGTTVYGH |
| | | | MWNAFATGLRAGQRVAAGQLIGYVGSNGESTGPHLHFE |
| | | | VHPTVWAAGSQIDPAPWLRATRNPGGVPAPIPPPAPAPA |
| | | | PAPPPGGNTVGDPVWLPDALRPAVSNLVEYPGWRSRGH |
| | | | GDFKDIRGVMVHHTGGPASARSIADGRPDLAGPLSQLHIS |
| | | | RDGVVTIVAAGVAWHAGSGSYPWLPTNMGNWHLIGIECE |
| | | | WPYGEPGINDRNAHTVPWRRPQIIAIRNTCAALLQWMRF |
| | | | GVDRLIGHKDYAGRAQGKWDPGNMSMPWLRGEVEKDM |
| | | | SGFVFPGEDGPRPIIVDSGLPPVPLPPAPAPAYAGVLLHR |
| | | | GMNGPAVRRLQEVLRRWYSKLAVDGDFGPATEAAVRDF |
| | | | QRLRPPLAADGVVGPATAQRLGL |
| UEM46538 | JuicyJay | 35 | MQVLGQEITKVRVAGADAARVRIFGEDAWTAEPPTPVPD |
| | | | PVSYFDWHEGSGSTTTSIVSSHVLTAVNPSAAWDGESAA |
| | | | GQFEGDIGDVATSTWSIAVEFTLSAGSSWRNILHTSGAGA |
| | | | ADEVWLQLNGSTLSIWTSAGASTGFDPAQPTLPPNLPIQL |
| | | | VMTNDGTTQRVYVDGELVSSRARNNILEPTSAVIMDTSEK |
| | | | LNGYVHSLRFWDVVLSDVEVEALMPDSGDEEDPPLFRG |
| | | | MTDEITQYGITWTFDQEYMAGQFITGDWWVVGPVTVTSV |
| | | | SPAPQGRLDGEDDDDLTGEAGQRVDDRMRHGSMVLTDV |
| | | | TTDQAYDSRCRFYNPARAISYPYNLAANRSLISSVSAVHP |
| | | | GGSEMGDLADGAEGRVMETAAVLTCLDEEPPEDAFRPC |
| | | | YWGADKSKIVRASDIQWDRLANVTPASSPPTLVEYARFFE |
| | | | RPWLDHMQAYWVGQFLWPWKNQPGYGRGYAAVVGTA |
| | | | GLLVNCNYTEAQKRPIVHGLIQLGIDLAGMLKADSCFRVS |
| | | | GGHGSGRKFPVVFAHLMLDDDPYFQFEADAVGADVGED |
| | | | GDGEPQALFGEDASTYWGETYYGAPACFQMVGYSSEVP |
| | | | PHQESPPPYTDWDDTSEGYRQCCTVGAWVGQTLAILLM |
| | | | GAKAAWNHDSYFQVVDDWMADPDPYAANRGGHPRPDD |
| | | | WQETNAWHPWATRMWNLHRSSVPAQDDGADNYKWMA |
| | | | LDREWVPNPPPA |
| YP_002225043 | Myrna | 36 | MTTPNVPTGNVTPPGPQPGTGYSQTTPDQEQGVDNYYL |
| | | | TDVGGKEPPPMETLVYTPRVRIIISRDNKQYDVSEDVVAV |
| | | | SINRVENSVSSAAFRLINKPEDQYDENHRQGRYTPLFAPM |
| | | | DRVAIFMKRTEWVQVFSGYLDSVALAQLYPGTVDFKASC |
| | | | TLKRLLHTWWDPGLPDSAKLFDQFANDVNEADGQESQL |
| | | | DKGLGSTLRRLLIEVGNMDSSQIHIQRFPMGYLNFMRQQL |
| | | | EKLQSSEKSYMEFRRMLLGDDTSGGVGAAAGRQLGVTK |
| | | | GAYNVDQAGRKLEVIRAVDEMGMGPDLRNIGLGQGLGTT |
| | | | SGAGKDLEDQEAWKLQQELGRNYTEAAQKNDAAVHCFM |
| | | | TIMAESSWIMYANTAVPESLNFPHEAVGHDHDSVGLFQQ |
| | | | RQTWGTIDQRMNPRESAGMFLNELKKHDWRNMPRPAAC |
| | | | QAVQRSAFSDGSNYAVHEAVAMEEVRALRTSGAAPSPG |
| | | | GQQSTISPVGTAPPPITAGVPGSTGIPGIPTVNGQASTQG |
| | | | ANQALNRPQYDLAEAVQFGMMQQGKPYQWGAKGPNSY |
| | | | DCSGFTRACYRYIGLNIGDDTYSQVASGTKITMAQAKPGD |
| | | | LLFPAGHGHVVMYLGGNQILHAPQTGDVVKVANLYFTPAT |
| | | | VCTYPPATYSGPSPIPYDPTRAAQASNGGSDGGVVPGTA |
| | | | AQGTNGGTYQTGSSEPIARNLFSYMFEPGRFQSGISALF |
| | | | GMDEGQTEKAFINDEPLIQSVMSIARAGLRNVQSMPDGS |
| | | | FAAYYPDYFGLDGKDAILDLEDIEMKNVNINWNDDALATH |
| | | | VYVAGTANPTGGSMGLPGWLMTKGVATVENEWLFRRMT |
| | | | AAAPAVKGAEMRNGKEIMKRFGMRPLVQDMPNLFNGEM |
| | | | EFLMALQIFMTKWAEQYSTQVEFTFMPELYPGMRINLVGH |
| | | | NLQVYVAAVTHSGDWENGFTTSATIMAPSNPNIHRIAQKI |
| | | | DSDLQFGTHWDQRDIDGKPMPSDYSWMY |
| YP_002225120 | Myrna | 37 | MALITENGWPQVPRSLCDNLLVPGTNRVKPELRKDDVTIIL |
| | | | VAWACWFDRNVRNIEPPDGHRNWWAWSATNDVWNSN |
| | | | HLSGTALDLCADELPWQRRTMPQNQVEITNRGIALFEGTV |
| | | | FWGRNWSRVDEMHFQIGLPPSSPKIREFADRLRNGYLNIF |
| | | | GPPDPNAFPLPLGYYYGPLDGPVESISGEYESDSQAAKD |
| | | | GLGRWQAALGLPVTKKWNDGLTPRAAYVLQRTKGWPAN |
| | | | PLFGFGGVYKGEWDAVIREGWRLPADVNLHNVVIPEFEY |
| | | | PLTKWGDYSQYQAATVDNTYPYEVISFRASIANQIDEKWL |
| | | | ANMKAAQILVRQGKLKKIIAYHFWVPGADNWGTFRRAIEA |
| | | | AGGVTKELCFMLDVEDGGTKWNIRGDQTAGVKSFIASGQ |
| | | | TYFQNPQAASIYWNPTANPDLLVGINDRELRGVKLIVPRY |
| | | | NGPDKAPWTPDGVQWFGHQYSDRENTPPFGPTDINQAK |
| | | | MPLSIFLAAWGTNGGVAEPDDPGTPVPAEPVPPVDTDSG |
| | | | EAEPDWSSERVLVAIGAQFGA |
| AYQ99975 | Nebkiss | 38 | MTNDVYAREIIRAGKDQGITPRGIVIAFATVFVESNWKNYA |
| | | | NSKVPESLALPHDAVGSDGKSVGLFQQQVVWGNGWWW |
| | | | GDAATCMDPYKSAVLFFQRLKKLDYNNTSRSPGSYAQDV |
| | | | QKSAFPDRYGQRMAEAQRYYDRLTGGSAALTPEPGFSG |
| | | | DPWWLAEVLRDEGLRVFEVDGWQNRGQGDQGRFWGV |
| | | | VFHHTGSPSETPEGIAFHPTLGLAAHILIRPNGDVWVCGIG |
| | | | KANHAGKGSWWGVPTDNGNPVLLGVEVAILPREGAPHR |
| | | | SGWPDVQYDATVKVHAALLRKLGLGSDRVISHKEWAQLG |
| | | | PAGWRQGKWDPGAIDMNVFRADVQAQINSKTGDDPLAD |
| | | | PDVVKKINEIHACLFNQIPSKSKYRDLNEGNKWQLHQLIGN |
| | | | DDAMIHEMLVERQAMMGNPEALALVKREADKGDKWAAV |
| | | | VYQYCTEAPE |
| YP_009955044 | Bubbles 123 | 39 | MSFTWFRPEGPLRTREQIAREVHAVSLARGLDELATVIAL |
| | | | MTISTEVGTGTGDDRKWWCPANDRVPATKNYPHDSRSD |
| | | | DNRSSGYFQQQPGPNGEPWWGTPENMMTLPQAANTFL |
| | | | ERLSDDYRRAANNPRLAGEFAQRVQQSAYPDRYADKWD |
| | | | EAWSVLRRALNETTPEEPVTENRPAYNEFPIWSANNSAR |
| | | | SGKPTMFLIHTQEGGGGDAAAENLAKWFQNANGVSYHY |
| | | | TISQASDGGVTVVDCVDTDRAAWSVGNANSISINLCFAGS |
| | | | RASWMRDQWMKQSNAIDVAAYLAVQDAKKYGFEPLVVP |
| | | | PPYVNGRPGISDHRWVTDVFKWGTHTDVGDWFPWDYFT |
| | | | ERVNHWANGGKTEPEPPKVKRFPDDWTDREILVEILRQL |
| | | | RGYNLTGWPQLGGKTLVDAVADLRTDIIDLQGAIEHGEITL |
| | | | GGAQ |
| AVO21346 | Megabear | 40 | MAVARANVEATKTFIRARLGNPYVYGGALSPTNVKQGTD |
| | | | CSEIWQTTLEMTLGRYVPGRQGEGATTESYRPKSMGGPI |
| | | | PDGGIGPFGTIVTRDRNAVPANAVAKIGLHHGPGGGANS |
| | | | HMWGELDGMGIESRGGGVGVITNPRSMRWDDSYANAFA |
| | | | YLPGPITEDGTPAAPEYVLLGRNYENSGDRVRQLQRALN |
| | | | SYGYGLDEDGEYGPLTEQAVGAFQRSKGLEVDGIAGPVT |
| | | | LAALGLTFGRPVESSPEGLALFRQIMSRTDATNAWLATCL |
| | | | PYYIEAMRAAEINTPLRAAAFASQIGHETSGLKDMAEIQTN |
| | | | GPGWTEDRRIYRGRGAIQLTWSSNYRRFGQWCREQGHV |
| | | | TDPELFVKNPELVEHPQWGFLAASWYWRFGGPKPGQIN |
| | | | SYADKGDILAVSRCVNGWIDGVDPVGWADRRSRYLNCM |
| | | | ALGDRIMNLTTVDPLEELLKMKLNSMSIYRTPGEGPIDAAI |
| | | | MLAALDAHGPHEDYVEKMARRGDEDALFRVARVAMGKG |
| | | | AVTTPAAIKQATDVLLEIAATNRAAVNEALGKV |
| YP_010051635 | Mercurio | 41 | MADRFAPMRLGTYTLASGFGPRWGSMHRGLDFAAKDGT |
| | | | PFYAAQAGTVVYIGPADGFGQWIVVDHPAEDGAGATVYG |
| | | | HMWNAFATGLRVGSRVKAGQLLGFVGSNGQSTGPHLHF |
| | | | EVHPYSWRAGSQIDPSPWLNGAKNPGDPAPAPAPAPAP |
| | | | KPAPAPPPPGGRMLDPFTGAVWSPNRSKRSKGNPRWIAI |
| | | | HTQEGGRTARDLAEGWLAKPASQVSYHAAVDERERLKIV |
| | | | AESDRPWAAANANDYAFHVVAAGSYAGWSRGKWLETDA |
| | | | SDGKNEDAELTSLAMVVAWWCEKYAIPAEWIGGRGIPWG |
| | | | RDGICGHADFGQWGGGHHDPGAGFPVDELIRRTRGFLA |
| | | | NGSAPAPLPAPAPVIPRGVEPVPGVLLYRGRIGQNPDQVR |
| | | | ALQTALRKWYSKLAVDGDFGPATEAAVRDFQHLRPPLVA |
| | | | DGIVGPATAGAMGLRF |
| UVT31471 | Sejanus | 42 | MPILRANVDYAFQIARARNRKPYGYGGVWVKNDVNRTTD |
| | | | CSGIVTHVLDALVNGERMTWSRHGLSTEAYRYVGGPGSR |
| | | | GPFGTIRVARPGDIPADAALRIGLMHGPGGGANSHMACTL |
| | | | EGVAIESRGGTVASGGGQWVGGSGRHYNNPLFHDWFYL |
| | | | PGPIVGTGTTTPTTPTAPGAVYLGRDCSRYECTGERVRAL |
| | | | QARLNRDYAAYSDLEEDGEFGPLTEAVVKQFQLRSALTA |
| | | | DGIAGPATLAALGLSFQPQEVPPVTAPQPVIVGPADDQLT |
| | | | MRWNALGGKTLVEAVAEIRDAVLGTEDRGKPGVVTK |
| QIQ63123 | Settecandela | 43 | MTEKVLPYDRAVVPQEYGWSCGPAATQVVLNSRGIVVSE |
| | | | TSLLNQIEAIENPGRGDDRDGTDYVGLIERVLDNIVPDARY |
| | | | TSVYLEKDPPTAAQKDLLWTHLKRSIDAGYGVVMNWVAP |
| | | | PSNKPRGVKGSVSPRYSGGTTYHYVAAMGYDDNPALRA |
| | | | VWIADSGFQPQGYWISFDQCASLIPPKGYAYADVTVTPPP |
| | | | AGSNADLVAILAQAMSPSSMPRETFENYLPFFAEAMRAAE |
| | | | INTVRRAAAWCSQVGHESGGLRYMAEIQTDGPGWTEDR |
| | | | KRYRGRGPIQLTWSSNYRKFGIWCKEKGYITDSEIFVNQP |
| | | | ELVEQPKWGFLASSWYWLFDGPKPGQINGYADAGDNFS |
| | | | VSRCINGWITNDDGSARTPNGWDDRQARYNRCLALGEQ |
| | | | LLKLTDAAGGDTELSAEAERMIREMYQEWRKEKIGPSRSF |
| | | | LAPDSNPIESPLGFIYNIDGNEWNGLNVWAYLFGVPFAVE |
| | | | AVERVAREGVHPKSYAATVPFVAEFGQAFCKGLVEFKAK |
| | | | LMPLLEGFDVQALRAQVEKPKADPEPWTGMSGFDDF |
| YP_010062396 | Stasia | 44 | MARRLFRGRQFSENGWPYVDQGSCTWDEVVPGVWLQI |
| | | | QNGAPFTIMRAFARDFHAHVEPLRDYDSACWTQDNSVDT |
| | | | SNHPGGTGMDLNWNGADQKTFRYGITKERAYPGDKARK |
| | | | LDELLAFYEDVIYCGGYWSIRDWMHFQMGYGTYDSKADR |
| | | | PTQKTLDFIARKIRPDGFSTFNRGGGGSSTESAAAVLARA |
| | | | TGVTLTKAQSILPAVRDGLIQSECTTVPRIAMWLAQCGHE |
| | | | SAHFEATEEYQNGPMDQERWIYKGRTWIQLTWRSAYEG |
| | | | FGKWCHARGLVTDPMVFVNNPRSLADLKWAGLGAAYYW |
| | | | TTTVRSTRKYPTLNQASDARDVLVATQIINGGTNGLTDRQ |
| | | | NRYNRAIAVGDALLQIVQEEDGFLSALNPAEQRELLDLMR |
| | | | WVAAPEYGELRKLFANEDMYREDNVRRRTFAGVAIDART |
| | | | FGWEDRVEKSAERGELWAIDLVVRAARGTLPGVIRPGTN |
| | | | TPDQFLVNHARQVLADVERINPEALKKYLALEGGSR |
| YP_010109361 | Gail | 45 | MVTRANVEATKAFIRARLGNPYVYGGALSENVRQGTDCS |
| | | | EVWQTVLEMVHGRWKQGRQQEGATTESYRYIDVGQVG |
| | | | PFGTIRVAHWRDIPADAAARIAFHHGPGGGANSHMWGEL |
| | | | DGMRIESGGSKGLVTGDRAMAVESNYATAWAYLPGPIGG |
| | | | TGSLGIGSTGPAVLALQHKLNERGAGLDVDGDFGPLTASA |
| | | | VTRAQQELHVVGDPPGVAGEATLRALGLLGADGAIPAQP |
| | | | NVLAAEYLAEATGVSVQKAQQMLPTMRDGLKQADATNVN |
| | | | RIAMFYAQTGHESANFNATEEYASGAAYEGRHDLGNDYP |
| | | | GDGVRFKGRTWIQITGRHNYGEFSKWAHWKGLVPSPTY |
| | | | FLDHPQELSDLKWAGIGAAWYWTVQRPDINALSDRRDLD |
| | | | TVTRRINGGLNGIDERRTRYNRALAMGDKLLTVLASNEPQ |
| | | | DEWDALMADQTRYPSRAWYRSDDVPAHTPLDLLRNIDG |
| | | | MKWDERVTQAAFRGEVWAIEQIAKVARGEGPEKSQAAIE |
| | | | ESKNLVRQLKAALAAQIGDSK |
| QOP65589 | Suigeneris | 46 | MTERVLPFDRKIIRQDTGYWCGPASTQMALSARGKYVDE |
| | | | ATLARECKTTVNGTDNVGLIERVLDVRLPEGNYTSMYPG |
| | | | GVKIGSPARPAAERKTRFWWDIVRSIDNGFAVILNWVVPP |
| | | | ARKPIRAVKGSTNPSYGGGTTYHYVTAVGWSDEGNNGR |
| | | | PAVLIADSGFAPNVYWVDLDTAFALIHTDLWKGYAYADLP |
| | | | LIAPPPPGVEVPPGIPVKPGTPPVAVTPPVPAPPTQPPPTK |
| | | | LGSLTDPFTGALWSPNHYDGRGGLGTPGWIAVHTQEGG |
| | | | RTARDLALFLANPANEVSYHSVNDDIEVLKCVAETDAPWS |
| | | | ASNANKYAFHHCFAGSYAGWSRDKWLSPDASDGKNEDV |
| | | | QLTKGAHVVAWWCDKYGIPAEWIGGRAQPPWGARGILG |
| | | | HVDLGQWGGGHFDPGGNFPVNEFIRRVVTFLTGEVQPPL |
| | | | VPLPPVVQPGTNPDAYSDWMLVRGDPRNDVDRVMRVQS |
| | | | RLKRAYAAYAGHLDVDGDFGPATQAAVREFQRRSNLVAD |
| | | | GIVGPMTAAALRP |
| YP_009013130 | Avani | 47 | MDINIMRQALSPTSASDTWLAECLPHMEEAMRAAQINNP |
| | | | RRASAWYSQIGHESAGLRYMAEIKTSDPSWSWDRTRYR |
| | | | GRGPIQLTWQGNYRKFGQWCKDQGYINDPELFVNQPEL |
| | | | VEQPKWGFLAASWYWLCAGPRPGQINGFADVGDIVAVS |
| | | | RCINGWIETKLPNGMPDRQQRWDHCLALGDAIVPGNITPP |
| | | | KEIPVTWTGDPVWLEEVLRPALGDRLKTLPGWQNSGHG |
| | | | DFKDIRGVMVHHTGNSRESAQSIRNGRPDLAGPLANIHIA |
| | | | PDGTVTIVAVGVCWHAGQGSYPWLPTNNANAHMIGIECA |
| | | | WPTPRPDLPKGYDPAERWPDAQIISMRDTCAALALKLELP |
| | | | ATRVIGHKEWAGAAQGKWDPGNLDMSWFRGEVRKDME |
| | | | GFVFPGEHPPIEPQPGPTLPPDYAKETWDQLRIQWPQLG |
| | | | GRTLVDAVAVIGEKLGIEGFYDVKKTQS |
| YP_010051498 | Avocado | 48 | MGSENGWEPARLAPDSPLLVWKIVPGTNPPVHLQVMRG |
| | | | FPEVFLIAWAADWNEFIEPLRDADSACYTPTNSVSTSNHL |
| | | | NATAEDLNWNSHPFRKRGSLNAQQMAVLKEMEDFYEGW |
| | | | MFWAGRWQNPIDEMHSQCGYGTWNDNDRGFDFIHRKIR |
| | | | SDGRSTFRRGSLPGENLPAAAPAFPPIAPAHDDEVAATVL |
| | | | YDAVPIIDMDRARKLLPLVRAGLVAAKCDTPRKIAMYLAQV |
| | | | GWESDGFNATEEYAKNGRYAPFIGRTWIMVTWQSNYAA |
| | | | FGRWCYDRHLVSDPDVFVKNPRKLADDEWAGLGPAWYI |
| | | | TDARPNINAMADAGDLLGVTKAINGGTNGLEDPRPGVPG |
| | | | RRTRWNQAIALGERLLELINHPDTEDTMPGLTDDEQRELL |
| | | | VNTRWLREQLEVSRPDWSPDADLGTDSQGRPNTLRTAV |
| | | | AKILRLVDKKPAAGPSSTPPATS |
| ABE67284 | Halo | 49 | MADRFFPMRDGTYTLSSGFGARWGTQHRGLDFAAKDGT |
| | | | PIYAAQAGTVAYIGPAQGFGQWIVIDHPAADGAGTTVYGH |
| | | | MWNAFATGLKAGDRVQAGQLIAYVGANGQSTGPHLHFE |
| | | | VHPTVWRQGSQIDPLPWLHGSRNPGDAPAPAPADPPAS |
| | | | APLGGQRMQDPFTGEVWSPNRSVRQKPAPRWIAIHTQE |
| | | | GGRTARDLCEGWLAKRESQVSYHVAVDDREILKVVAESD |
| | | | RPWAAANANDYAFHVVAAGSYAGWSRGKWLETDASDG |
| | | | KNEDVELTNLAKVCAWWCQKYGIPAEWIGGRGVPWGLD |
| | | | GICGHEDFGAWGGGHHDPGPGFPADELIRRVRALLGGST |
| | | | PEPLPPAPPVALPGTNPDQYAGVLLYRGRPGQDPRQVRV |
| | | | LQTRLKRAYSKLDVDGIFGPHTEACVRDYQHLHPPLVADG |
| | | | IVGPATAAALGLVF |
| ASM62633 | Miley16 | 50 | MAFVSRYGNWYSENGWRMCDANELDRGAVPGTTLVLPI |
| | | | RKGIANLILKAWVAWFHRNVESLNNARGFSDEGAWTPTN |
| | | | DVANSNHLSGTAVDLNWSDHAFRVSYSGFTQAEINKVRE |
| | | | GLRLFEGTIWWGQDWTSPKDPMHFQLNYGEGDARNAAF |
| | | | ATKLQNGYLGIWSGGGGVIEPPAGVDDIYAQEGDSGDRV |
| | | | KRLQQFFNDNFKSYSQLEVDGDFGPATKAVVVEFQKRVG |
| | | | VLADGIVGPITLAAMVKHGFKTESGVKVANLPEDWTDRQV |
| | | | LNDIWEQLRGPDGKGWEQLGKNAKGENLSLVDAVAEVK |
| | | | KRLEAA |
| QBI98680 | Bobby | 51 | MANRVVYGNSFSSNGWPMVDQGSCTWVKIPGTSVTLQI |
| | | | QNGQPLAILRAFAADFHAYVEPLRDADSACWTPTNSVST |
| | | | SNHLSGTAMDLNWNSHPFQVPDAGFDAAKKARVKELLDF |
| | | | YEGTVFWGNDWTSPKDAMHFQLASLRNGGNINTYGNPF |
| | | | VDDFISRKIRPDGFSTFRRGDSPPADVAKVLADATGLTPA |
| | | | RAREILPMVTDGLRAAECTNVNRIAMWLAQIGHESAGFNA |
| | | | TEEYDHGRNHGDPNEVTDRWKYKGRTWIQITWQSNYAG |
| | | | FSKWCHGRGLVPSPSYFVDRPRELAELKWAGLGPAWYW |
| | | | TVARADINALSDKRDLDTVTRRINGGANGIADRRNRYNRA |
| | | | LALGDQLLSLISEGGDDLTPEQDRMLREVHACLFNPISSQ |
| | | | SKYKAEGEGARWRLHELIKNDDALLHETVVERQAMMGNP |
| | | | EALALVKREADKGDKWAQVVYRYCTEEDA |
| YP_009200654 | Bipolar | 52 | MTTKDQVAQITIAEAKARGYTRSECLAIMSTFYQESGWND |
| | | | TIWDPTHTTYGIAQQDGSYPHRFDGAAAQIKGFFDKLDV |
| | | | WRAKPGASTDIWLNICWMQQAPNWPSADYWYANGRRA |
| | | | YLTEIKSRIATVTPYLDKYWPTTGGTDVPETRPPYNEFPIW |
| | | | SENHYNTKRTVNDIDAFLLHTSEGFVGRDDAAEALSLWY |
| | | | QPRSRQVAYHYAVSQASDGGVTVVDNVDTDYASWSALS |
| | | | ANGRSINLCFAGTRAAWSRNEWLGKFGNAIDVAAYLAVQ |
| | | | DCKKYNIPTKVIAPPYTGRLPGITDHRYVTQILKDGTHTDV |
| | | | GDGFPWDYFTERVNHWAAGGKTDPEPPKVKRFPDDWT |
| | | | DRELAVETLRQQRGYTLNGWPQLGGRTVVDVLGAIGAKL |
| | | | GVEGCYDVKDKS |
| YP_009125981 | Malithi | 53 | MPILRANVDYAFAIARARDKKPYGYGGVWSKTDVNRTTD |
| | | | CSGIVTHILDALVNGEKMAWSRHGLSTEAYRYVGPAGSR |
| | | | GPFGTIRVARPQDIPADAALRIGLQHGPGGGANSHMACTL |
| | | | EGVAIESSGSYGQRVGGPARGYNHSMFHDWFYLPGPIV |
| | | | GSGTSTPATPTAPGAVYLGRDCSRYECTGERVKALQARL |
| | | | NRDYPAYSDLDEDGEFGPLTEQVVREFQSRSNLTIDGIAG |
| | | | PATLAALGLSFQQQPAAPAPAPAPAAPKPVVVGPADDQL |
| | | | TMRFNCLGGQTLVEAVAEIRDKVLGTDDRGKPGVVMK |
| AXH47159 | Cborch11 | 54 | MAFIQKQGYWKSENGWRMCDTAELDYTPVPGTNFKLGV |
| | | | RKGAPSVILKSLIWRLNRIEPMITSQIGCYTAENSMRNSNH |
| | | | NSATAIDYNWNLHPYQKWGTWGNNRAAVDKVIADFRGIV |
| | | | EFGGNWTSPRDEMHFELHFAEGHQGTEQLAQELRDGLW |
| | | | GIWKAGTAPSDAPSSGGGSSAGDILRIGSTGPEVLKMQK |
| | | | GMNAVFKNYKAMPLDEDGIFGPMVQAAVIEFQQRSLISVD |
| | | | GEVGPQTKAKLAEYGIVLTGQTTPTSPPAPAAFVYPSHEE |
| | | | MVKQLWEQAFGPQAKGWESLFGKSVDGSRGKFTVEAIA |
| | | | DIRNVATQ |
| UOW93095 | NoShow | 55 | MPWKGDPVWLPDVLRDWRVPFSISEGAFQRGHGDMGDI |
| | | | WGVVDHHTGSDNASWQSIAFHPSLGLASQLHLGFNGHV |
| | | | TICGVGIAWHAGNGSWPGLGTNNANPRTIGIEAANDGGG |
| | | | KPGRPHRADWPVAQYDNYVKMNAAILSFLKYGSDRSIGH |
| | | | KEWAGAAQGKWDPGGIDMNIFRRDIQSLLTIPAKPLVNAI |
| | | | DEHAKVAGAWLGKRVTSGEQPTADGKGRFAQFENGYIY |
| | | | WSPDTGAWAIPRHLFETYAELGYEAGPLGYPIAPHDVVE |
| | | | GGDVQAFQRGVLYRKYGESGHFITGAIGNRYRLNQYERG |
| | | | FLGWPTTNETKQGGMIWQGFEHGRIAFSPDGTIVLNERN |
| | | | DFV |
| QXO14766 | SmellyB | 56 | MALGAPMENGWPECDLSDCDYATVPGTPLRLPFRKGHP |
| | | | FIILQAFLRDLNEFVEPLMNARGATDEGSWTDNNSVYTSN |
| | | | HKGATAFDYNWSDHPMGNALAGWHGSVLISGEQEPAVR |
| | | | ELLRYYTYRGIQLVWWGNDWYSPKDSMHFQMGYDTVNN |
| | | | PGIVQEFIDKFIRPDGYSKYRRDGSSGPVELTVPLIPAANG |
| | | | RWTSPSPAWAHLITRESGGNPTIIQQIHDVNSGGNEAEGL |
| | | | FQITPRTWKAHNGTEFATSARFATPQQQAIVAARIITRNPS |
| | | | GSDWGAGLPGREDAAQLRAGLVPTQTTNDPLEELMAME |
| | | | VESFSIYANPGEPKIPVHVMIQSLDAHGPHEPYIEEQARHG |
| | | | DRDAIFRVARTAAGKGKYGDAPGPVKQASRVLKELEEAG |
| | | | VLAEYLKGN |
| YP_009204217 | FlagStaff | 57 | MGSENGWEPARLAPDSPLLVWKIVPGTNPPVHLQVMRG |
| | | | FPEVFLIAWAADWNEFIEPLRDADSACYTPTNSVSTSNHL |
| | | | NATAEDLNWKNHPFKQRGSLNAAQMAVLKEMEDFYEGW |
| | | | MFWAGRWQNPIDEMHSQCGYDTWNDNDRGFDFIHRKIR |
| | | | SDGRSTFRRGSLPGENLPAATPAFPPIAPAHDDEVAATVL |
| | | | YDAVPIIDMNRARKLLPLVRAGLVAARCDTPRKIAMYLAQV |
| | | | GWESDGFNATEEYAKNGRYAPFIGRTWIMVTWQSNYAA |
| | | | FGRWCYDRHLVSDPDVFVKNPRKLADDEWAGLGPAWYI |
| | | | TDARPNINAMADAGDLLGVTRAINGGTNGLEDPRPGVPG |
| | | | RRTRWNQAIALGERLLELINHPDTEDVMPGLTDDEQRELL |
| | | | VNTRWLREQLEVSRPDWSADADLGTDSQGRPNTLRTAV |
| | | | AKILRLVDKGKPATSVANANPPATS |
| AXC33829 | Tarynearal | 58 | MSFRTAYGNAYSENGWRMCNRDECVTVSGPYMNTAPLR |
| | | | RGPAEKLLGEFVRRYHQVCAPVVSPVWGWSETNDVGNS |
| | | | NHLSGTAVDVNAPQWPWGLRKMPADLVARINVLLDQFEG |
| | | | AIYWGRNWNRPDEMHFQLNWREGDAKYDRIIAKFSGGS |
| | | | VPVTPHVPNLPTDDSELLMRGSANVEQTRILQAGLKKVFP |
| | | | SYAGHLEVDGDYGPETEKAVRRFQSGSGLVADGIVGPAT |
| | | | RAELAKYNIILKPAAPQEIIPVGKTPVVVGPADDQLNMRFN |
| | | | CLGGKTLVEAVAEIRDAVCGTNDKDKTGVVLK |
| YP_009304666 | Bactobuster | 59 | MARRLFRGRQFSENGWPYVDQGSCTWDEVVPGVWLQI |
| | | | QNGAPFTIMRAFARDFDAHVEKLRDADSACWTQDNSVDT |
| | | | SNHPGGTSADFNWNGADGRTFRYGITKERAYPGDKARK |
| | | | LDELLAFYEGVIYCGGEWSIRDWMHFQMGYGTYDSKADR |
| | | | PTEKTNDFIRRKIRPDGWSTFTRGGGGAPAPAADAASIVA |
| | | | RATGVTLAKAQEILPTLREGMVLAECTTVPRIAMFLAQLGH |
| | | | ESAHFNATEEYQNGPMDQERWIYKGRTWIQLTWRSAYE |
| | | | GFGRWCFARGLVSDPMVFVNNPRSLADLKWAGLGAAYY |
| | | | WTTTVRSTRKYPTLNQASDARDVLVATQIINGGTNGLEDR |
| | | | QNRYNRAIALGDELLQILGEEDSFLSALNPAEQRELLDLM |
| | | | RWVAAPEYGELRKLFANEDMYREDNVRRRTFAGVAIDAR |
| | | | TFSWEDRVEKSAERGELWAIDLVVRAARGTLPGVIRPGS |
| | | | NTPDPFLVNHARQVLADIEAINPEALKNYLKGAS |
| QPL14214 | BangNhom | 60 | MARRLFRGRAFSENGWPYVDQGSCTWVRIPGAEHVSLQI |
| | | | QNGPPLQVLRAFAADFHAHVEPLRDPDSACWTQDNTVD |
| | | | TSNHPGGTAMDLNWQGADGKTFRYGISEERAYPSPKHQ |
| | | | RLRELLDFYEGVVFCGGFWSIQDWMHFQMGAGTYDSRA |
| | | | DRPTEKTLDFIRRKIRPDGFSTFKRGGGGSAAPDAASVLA |
| | | | RAADIPLAKAQEILPTFREGAVLAECTTVPRLAMFIAQTCW |
| | | | ESDRYRATEEYANGPMNEERWIYKGRTWIQLTWRSAYE |
| | | | GFGRWCHARGLVNDPMVFVNNPRSLADLRWAGLGAAYY |
| | | | WVTTRRESRKYPTLNEASDARDVLVATQIVNGGTTHLAE |
| | | | RTAIYNRAIALGDELLHILGEEDELANPEIEKMIREVHACLF |
| | | | NRIVSQSIYRFPKNPDGSEHPGNIWQLHELIKNQDGMAHL |
| | | | KYVEDSARGGDLTELDRIAVVAAGRGAVRDQWAVQRAQ |
| | | | RVLADIERTNPEVIRRYLAQKGAA |
| YP_009853783 | Indlulamithi | 61 | MTEKVLPYDRSIVPQETGYWCGPASTQVILNSRGIIWKES |
| | | | DLAREIGTTTRGTDYVGLIERVLDRVTPDANYTSVYTERD |
| | | | PMTNDQKETFWRNIVQSIDGGYGVSTNIVAPPNNKPRGV |
| | | | LGSVSPSYSGGTTYHYIAIMGYHIGDDGFRAVWVADSGF |
| | | | RPFGYWCSFDQMATLVPPKGYCYANTGPVVPPTAPQIVP |
| | | | VALFSKAMGDTIGLDQYAALLPGFVDACEKLGVTENNEIA |
| | | | MLAAQFGHESVGLKYMRELWGPTADQLNYDHMMGNGP |
| | | | GEGRKYLGRGPIQVTGKDNYRELSRWAFDQKYIDSPTLFL |
| | | | DQPELLEQPQFGFLGAVWYIKVKQPRFMEYARAGDIENA |
| | | | SKAINAPAWIGTQNRARGIDDRIARWNRCRAMDLMPLLRE |
| | | | EDDPLSDPILAKLIGEIHGALFNPIPSLSRYREDYEGNALTT |
| | | | KDIPRNVDKFSHEEWTETAAIRGNPKAVKMVARNASRGD |
| | | | SMAMDVFARIPAETLAANGIDSAAVKSGYDKSQAEDPNR |
| | | | SVFALPPKGN |
| QDM55624 | HokkenD | 62 | MALYRTLYPLTYRSGNTVMHVTKPGVIIDLDRNQAQELSG |
| | | | RVVYVGDLITYPRSGVMYYDSVDSFPLEGDERYIFLARQE |
| | | | GELYGWSYSDGYKLFGAAGQVDEEALAEAVGDVIEDALG |
| | | | DALAGKANVSHTHNPSDVTGLPAAIAKLAGIDAGANVTNA |
| | | | TNVNAAGAVMNSDTSTADMQFVVDEDNMASNSATKVPT |
| | | | QQSVKAYVDSRVGGGGAAGVNYGISTLRIDTNDVPIADRE |
| | | | TYIEATFEIEGITYNGEIRGRGNSTWDFPKKPWRVRLDSA |
| | | | AALLGMPSSRHWALLANYIDRSAARNAIAMQIGSRLSGLD |
| | | | WTPKYRYVEVVLNGTYEGLYQLMEIVRFDPNRVDAEAAD |
| | | | DTTGLGLTGAYLLEIDSYRDADVVIDTVHDDLPIIMDDPDG |
| | | | SVTEQATYIEDWLNNFETVLYDDDEWLDPETGYKTLIDLD |
| | | | SFVDWYLVNELLVSIDAGFETSVKMYKTRDTIDTPGKLFLG |
| | | | PLWDYDQSMGRAGSTTFSHEGWWLLETTQPASGTPYPG |
| | | | ATWIVRMMSDPDFLDAIEVRWPQIVALLDDLEDLVTKTLR |
| | | | HIALGRVNDQVLWPDGGTWSTNASEIVDWLEDRMEWITD |
| | | | NLPALGVSDGEPPSIPEGLDYTATGSTINVTWEASTDNVG |
| | | | VTGYEVRIDEGTPVAKTGLSHTFTDLEPETEYSIDVRAKDA |
| | | | AGNWSDWSEPLVAETGEEGSLPSPISHFDWHEGSGSTT |
| | | | TSIVSGHVLTAAVPSSAWSGTSAAGQFEGDIGDTATPNW |
| | | | TIAVDFTLNAGSSWRNILHTSGAGAADEVWLQLNGSTLSI |
| | | | WTSAGASTGFDPAQPTLPPNLPIQLVMTNDGTTQRVYVD |
| | | | GELVSSRARNNILEPTSAVIMDTSEKLNGYVHSLRFWDVV |
| | | | LSDAEVEALMPDSGDEEDPPLFRGMTDEITQYGITWTFD |
| | | | QEYMAGQFITGDWWVVGPVTVTSVSPAPQGRLDGEDDD |
| | | | DLTGEAGQRVDDRMRHGSMVLTDVTTDQAYDSRCRFYN |
| | | | PARAISYPYNLAANRSLISSVSAVHPGGSEMGDLADGAEG |
| | | | RVMETAAVLTCLDEEPPEDAFRPCYWGADKSKIVRASDIQ |
| | | | WDRLANVTPASSPPTLVEYARFFERPWLDHMQAYWVGQ |
| | | | FLWPWKNQPGYGRGYAAVVGTAGLLVNCNYTEAQKRPI |
| | | | VHGLIQLGIDLAGMLKADSCFRVSGGHGSGRKFPVVFAHL |
| | | | MLDDDPYFQFEADAVGADVGEDGDGEPQALFGEDASTY |
| | | | WGETYYGAPACFQMVGYSSEVPPHQESPPPYTDWDDTS |
| | | | EGYRQCCTVGAWVGQTLAILLMGAKAAWNHDSYFQVVD |
| | | | DWMADPDPYAANRGGHPRPDDWQETNAWHPWATRMW |
| | | | NLHRSSVPAQDDGTDNYKWMALDREWVPNPPPA |
| YP_009032264 | Hosp | 63 | MALGMTLENGWPECDLSDTERLTIPGTALSLPIRKGQPHA |
| | | | ILQAFFRDVNEFIEPVMNARGLSDEGSWTENNSVYTSNHK |
| | | | GATAVDWNWSDHPLGVKNGGWDGSVLINGSQVPAMRE |
| | | | LLTWYEGMVFWGNDWSSPVDSMHFQMGYNTCCGGDNA |
| | | | KRVDSFIQRKIRADGFSTFRRGGTPRGGGFAELPAAPVHP |
| | | | IKPTSGLTPEVLWRIAGGAASKLPVSHFERWFDELVECQA |
| | | | ACGVLGNIDRSAMWYAQVFHESGNLVHTEEIASGAAYEG |
| | | | RCEGLGNCQPGDGVRFKGRSFIQVTGRSNYTKLSGWAH |
| | | | SKGYVPTPDYFVVHPDQLDDEQYAMLGVTWYWTTQRRM |
| | | | NDAADARNLELATRYVNGGTNGLDHRRAIYNRALAENAN |
| | | | LLLTNPVEPWEELMATAVPSLSIYANPGEADVPLAVMIAAL |
| | | | DAHGPHEPYVERQAQEFGDVDSIRRIARTANGQGRVKTP |
| | | | AAIKQATDAFRLIPPEFVRAAIPA |
| QBI97493 | Hughesyang | 64 | MAVTRANVEATKNFIRARVGNPYVYGGALSPTNVRQGTD |
| | | | CSEVWQTVLEMVHGRYNPGRQSEGATTESYRYIPVGGV |
| | | | GPFGTIRVASWRDIPADAVAKIAFHHGPGGGASSHMWGD |
| | | | LDGMLIESAGSKGLVTNGRAMTIDNSYATAWAYLPGPIVE |
| | | | DGSPIPEDPNAVTWGIDISNHQGEMDLNRVKAEGFDFIWC |
| | | | KVSEGANYRDPFWPGNRDKARAAGLILAGYHYVRTGDPA |
| | | | AQAKTFVEHLGDKSIPAMLDFEDGSGNIEQFWAVKAEIEK |
| | | | LGVQVRLSYIPDWYWERIGKPDLSKVPGLISSEYVSGTGY |
| | | | ASVLYPGNSSNFWKAYGGRTPDVLQFTDRALVAGKSVDA |
| | | | NAFRGTPDKLRRLLGAGGDDFLSALSDAEQRLLFDRTNQ |
| | | | LWGALFNPIASLSKYRAEGEGEVHKTKDLVRNIDAMTHET |
| | | | LVERQAMMGNPEALALVKREADKGDKWAACVYKYCTEE |
| | | | A |
| UVK63413 | Baudelaire | 65 | MSAKDDYARAVIAEGKRRGITPRGIQIGLATVDVETGFVM |
| | | | YANSKVPESLTIPHDRVGSDGFSVGLFQQQIVRGANGQW |
| | | | WWGDCATCMNPTLSAGLFFDRLAKLPYNDPGRSPGSFA |
| | | | QQIQQSAFPDRYDQHFAAAVDLYNRLEGDVVVDRPDFNE |
| | | | YAVWSPNSQSRNGAKIDLFLLHTEEGNSNADQLANGILSD |
| | | | PAPGGNPANAVSYHYTISMDPNDKGVTVCDVVDTDEAS |
| | | | WSVGNANNRSINLCFAGSKAAWTRQQWLDNAGKAIDIAA |
| | | | YLAVQDCKKYGISINVAPPPYNTGTPGISDHNYVTTVLKW |
| | | | GSHTDVGPNFPWDVFTAAVNKYADTGTVATPINPPAFTY |
| | | | PSTDVMIREIWEQLRGPQAKGWSQLGGKSLVDAVASLTG |
| | | | GK |
| AVE00798 | Tesla | 66 | MSFIRSAFADKPLLTREQWMKLFIRVADELDMPDRRGAAV |
| | | | LAAMCAFQEAGADPHNTGKRQIWVPGNNADPCFKANPA |
| | | | AYPHDSMGDDGQSTGPFQQQMNKPGTTPWGWGGNYG |
| | | | DPEGTRKRMDPYESTKMFMGHPSSGLKKKGYDASNAQT |
| | | | ANDAIQRVQGSGVPWAYAQWWDEANLLYDEVVGDVPPS |
| | | | TGGGKVPVSGDPVWLEEVLRKRLGDRLVVHDGWKEYGT |
| | | | GGVMGEIWGVMIHHTGNRNASWESIRNGRPDLRGPLSQ |
| | | | ALIAPDGKFHLVAVGPCNHAGVGSYPGLGSDGNRRAIGF |
| | | | ECAWPTIRPDGSFDKGERWPDAQILTMRDATRAVLEHLG |
| | | | HDHTHVCGHKEFNKVDGKWDPGNMDMNWFRGEVRKAL |
| | | | AGEFDPKAPEPEPQLPSQPGIPTDHDLLVYDQICGRWEM |
| | | | LGWRTPVEALATILDELRGTKNAGSRGFTRGPHDIEDKK |
| YP_009018052 | Thibault | 67 | MANRVVYGNSFSSNGWPMVDQGSCTWVKIPGTSVTLQI |
| | | | QNGQPLAILRAFAADFHAYVEPLRDADSACWTPTNSVST |
| | | | SNHLSGTAMDLNWNSHPFQVPDAGFDAAKKARVRELLDF |
| | | | YEGTVFWGNDWTSPKDAMHFQLASLRNGGNINTYGNPF |
| | | | VDDFISRKIRPDGFSTFRRGDAPTVDVAKVLADATGLTPA |
| | | | RAREILPMVTDGLRAAECINVNRIAMWLAQIGHESAGFNA |
| | | | TEEYDHGRNHGDPNEVTDRWKYKGRTWIQITWQSNYAG |
| | | | FSKWCHGRGLVPSPSYFVDRPRELAELKWAGLGPAWYW |
| | | | TVARADINALSDKRDLDTVTRRINGGTNGIADRRNRYNRA |
| | | | LALGDQLLSLISEGGDDLTPEQDRMLREVHACLFNPISSQ |
| | | | SKYKAEGEGARWRLHELVKNDDAMIHEMLVERQAMMGN |
| | | | PEALALVKREADKGDKWAQVVFRYCTEEDA |
| QOP6599 | Thoth7 | 68 | MAFIQKQGYWKSENGWRMCDTAELDYTAVPGTSFKLGV |
| | | | RKGSPNIILKALIWRLDKIEPMITSQIGCYTAENSMANSNHN |
| | | | SATAIDYNWNKHPYQKWGTWPNRAAVDKIVDDFRGIIEFG |
| | | | GDWTSPRDEMHFELHFAEGHAGTEALAKDLANGLWGIW |
| | | | KPGAAPAPTPAPGGGNDGILRIGSEGPEVLKMQRGMNSV |
| | | | FKNYRAMPLLEDGIFGAKTKEAVVEFQQRSLIDVDGEVGP |
| | | | QTKAKLAEYGIVLTGATAPTAPPAIVPQKVWPQTASDREL |
| | | | LEYIAAQLGPGDPSWPIKLGNNEKGDPLTVRDRLGLTAKD |
| | | | IEEIKGRLSQ |
| YP_009187186 | Sparkdehlily | 69 | MSFTWFADKPLRTREQVAREVHAVSLARGLDELATVIALM |
| | | | TISTEVGTGTGDDRKWWCPANDRVPATKNYPHDSRSDD |
| | | | NRSSGYFQQQPGPNGEPWWGTPENMMTLPQAANTFLE |
| | | | RLSDDYRRAANNPRLAGEFAQRVQQSAYPDRYADKWDE |
| | | | AWSVLRRALNETTPEEPVTENRPAYNEFPIWSANNSARS |
| | | | GKPTMFLIHTQEGGGGDAAAENLAKWFQNGNGVSYHYTI |
| | | | SQASDGGVTVVDCVDTDRAAWSVGNANSISINLCFAGSR |
| | | | ASWMRDQWMKQSNAIDVAAYLAVQDAKKYGFTPLVVPP |
| | | | PYTNGRPGISDHRWVTDVFKWGTHTDVGDWFPWDYFTE |
| | | | RVNHWAAGGKTEPEPPKVKRFPDDWTDRELAVETLRQQ |
| | | | RGYTLNGWPQLGGRTVVDVLGAIGEKLGVEGCYDVKDKS |
| YP_001552360 | Giles | 70 | MPVYAIQSGTVIYAGAASGYGGPDPAGWLVIDSDDAEGG |
| | | | GCLEYGHIVREVNRGDHVTAGQRIGHINPNNRTNGGVAP |
| | | | HLHVTDWPHAYGQGSRQNVMARLSGAREPEAQAAPTPT |
| | | | EQGTVVGDPVWLADVVRPTVAKFAEYPGWRNRGHGDFK |
| | | | DIRGVMVHHTGGPASAASIANGRPDLAGPLAQLHISRDGT |
| | | | VTVVAVGVAWHAGAGSYPWLPTNMGNWHLIGIECEWPY |
| | | | GEPGINERNAYTVRWRDPEIIALRNTCAAILLRLGLGVDRLI |
| | | | GHKDYAGRAQGKWDPGNMSMDWLRGEVRKDMDGFVF |
| | | | PGEDIVNVTPPPVPAPVPAPPATANVLLHRGMSGPNVVKL |
| | | | QTTLRRWYSKLAVDGDFGPHTEACVRDRQRIYGLDVDGI |
| | | | VGPLTAAKIGLVL |
| NP_818351 | Omega | 71 | MAVTRANVEATKNFIRARVGNPYVYGGALSPTDVRRGTD |
| | | | CSEVWQTVLEMVHGRYNPGRQSEGATTESYRYIPVGGV |
| | | | GPFGTIRVNHWRDIPANAAAKLAFHHGPGGGANSHMWG |
| | | | ELDGMLIESAGSKGLVTNGRAMTIDNSYATAWAYLPGPIV |
| | | | EDGSPIPEDPNAVTWGIDISNHQGDMDLNRVKAEGFDFIW |
| | | | CKVSEGANYRDPFWPGNRDKARKAGLILAGYHYVRTGDP |
| | | | AAQARTFVEHLGDKSIPAMLDFEDGSGNIDQFWAVKNEIE |
| | | | KLGVQVRLSYIPDWYWERIGKPDLSKVPGLISSEYVSGTG |
| | | | YASVLYPGNNSNFWKAYGGRTPDVLQFTDRALVAGKSVD |
| | | | ANAFRGTPDKLRRLLGAGGDDFLSALSDAEQRLLFDRTN |
| | | | QLWGALFNPIASLSKYRAEGEGEVHKTKDLVRNIDAMTHE |
| | | | TLVERQAMMGNPEALALVKREADKGDKWAQCVLDYIEGD |
| | | | ES |
| YP_009209509 | Cambiare | 72 | MGSENGWEPARLAPDSPLLVWKIVPGTNPPVHLQVMRG |
| | | | FPEVFLIAWAADWNEFIEPLRDADSACYTPTNSVSTSNHL |
| | | | NATAEDLNWQSHPFRKRGSLNAQQMAVLKEMEDFYEGW |
| | | | MFWAGRWQNPIDEMHSQCGYDTWNDNDRGFDFIHRKIR |
| | | | SDGRSTFRRGSLPGENLPAAAPAFPPIAPAHDDEVAATVL |
| | | | YDAVPIIDMDRARKLLPLVRAGLVAAKCDTPRKIAMYLAQV |
| | | | GWESDGFNATEEYAKNGRYAPFIGRTWIMVTWQSNYAA |
| | | | FGRWCYDRHLVSDPDVFVKNPRKLADDEWAGLGPAWYI |
| | | | TDARPNINAMADAGDLLGVTKAINGGTNGLEDPRPGVPG |
| | | | RRTRWNQAIALGERLLELINHPDTEDTMPGLTDDEQRELL |
| | | | VNTRWLREQLEVSRPDWSPDADLGTDSQGRPNTLRTAV |
| | | | AKILRLVDKKPAAGPSSTPPATS |
| YP_010062823 | Camperdownii | 73 | MARRLFRGRPFSENGWPYVDQGSCTWDEVVPGVWLQI |
| | | | QNGPPFTIMRAFARDFHAHVEPLRDADSACWTPDNSVDT |
| | | | SNHPGGTGMDLNWNGADGRTFRLGISKERAYPGDKARK |
| | | | LDELLAFYEGVIFCGGEWSIRDWMHFQMGAGTYDSKADR |
| | | | PTEKTLDFIKRKIRPDGFSTFGRGGSTAPVADGASVLARA |
| | | | TGIPLERARQILPALREGLILAECNTFPRIAMFLAQTCWES |
| | | | DQYRATEEYANGPMHEERWIYKGRTWIQLTWRSAYEGF |
| | | | GRWCHARGLVSDPMVFVNNPRSLADLKWAGLGAAYYWT |
| | | | TTVRNTRKYPTLNQASDARDVLVATQIVNGGTTHLAERTA |
| | | | IYNRAIALGDELLHILGEEDELANPEIEKMIREVHACLFNRIV |
| | | | SQSIYRFPKNPDGSEHPGNIWQLHELIKNQDGMAHLKYVE |
| | | | DSARGGDLTELDRIAVVAAGRGAVRDPWAVQRAQRVLA |
| | | | DIERTNPEVIRRYLAQKGAA |
| QGJ92181 | MarysWell | 74 | MCNRDECVTVSGPYMNTAPLRRGPAEKLLGEFVRRYHQ |
| | | | VCAPVVSPVWGWSETNDVGNSNHLSGTAVDVNAPQWP |
| | | | WGLRKMPADLVARINVLLDQFEGAIYWGRNWNRPDEMH |
| | | | FQLNWREGDAKYDRIISKFSGGSVPVTPHIPNLPADDSEL |
| | | | LMRGSANVEQTRILQAGLKKVFPSYAGHLEVDGDYGPET |
| | | | EKAVRRFQSGSGLVADGIVGPATRAELAKYNIILKPAAPQE |
| | | | IIPVGKTPVVVGPADDQLNMRFNCLGGKTLVEAVAEIRDA |
| | | | VCGTNDKDKTGVVLK |
| YP_010051763 | Pinnie | 75 | MTEKVLPFDRKIIRQDTGYWCGPASAQMALSSRGKFVDE |
| | | | ATLARECRTTTNGTDNVGLIERVLDVRIPDAKYLSVYPGGL |
| | | | KIGTPARPANERRDYFWWDVVRSIDAGYPVILNWVVPPN |
| | | | RKPIRGVKGSPNPSYGGGTTYHYVTCVGWSDEGNNGRP |
| | | | SLLIADSGFAPNVYWVDFETAFQLIHSDNYKGYAFADHPL |
| | | | QPPPAGVKVPPGVPIAGTAPTPAPAPAPPPAIVKPAGKIAD |
| | | | PGVYLLTKANRYENRGGKPWPFWIALHTSESRSRARDLR |
| | | | QYCETHEVSYHGIGDDREVIRMVRDEDGSWSAVGANNLA |
| | | | YHYCFSSSFAGWSREAWLDPNPADGYNEREALRLGAKQ |
| | | | VAFWIQLSIERGRPIPLEWIKGRGTPPWGHNGICDHSSFG |
| | | | AWGGGHSDVGHNFPVNTFMDDVRFWLTGTEAPPIAPAP |
| | | | PVVVAGTNPDRYADWLEYAGKPNPNRDRVAAIQAAMNR |
| | | | PPTPFGLDVDGIFGPLTRLAVIGFQQHVHLVADGIVGPMT |
| | | | AAALNP |
| AWY03479 | Erk16 | 76 | MAFIQKQGYWKSENGWRMCDTAELDYTAVPGTSFKLGV |
| | | | RKGSPNIILKALIWRLDKIEPMITSQIGCYTAENSMANSNHN |
| | | | SATAIDYNWNKHPYQKWGTWPNRAAVDKIVDDFRGIIEFG |
| | | | GDWTSPRDEMHFELHFAEGHAGTEALAKDLANGLWGIW |
| | | | KPGAAPDPAPAPAPGGGNDGILRIGSEGPEVLKMQRGMN |
| | | | SVFKNYRAMPLLEDGIFGAKTKEAVVEFQQRSLIDVDGEV |
| | | | GPQTKAKLAEYGIVLTGATAPTAPPAIVPQKVWPQTASDR |
| | | | ELLEYIAAQLGPGDPSWPIKLGNNEKGDPLTVRDRLGLTA |
| | | | KDIEEIKGRLSQ |
| QJD51348 | RawrgerThat | 77 | MSFIRSKFAPRALLTVDQWIAIFAAVADELNMPDKRGAVIC |
| | | | AAMCAFQEAGADLQDGNGRQIWIPGNMADPCYADDPDA |
| | | | YPHDSEGNDGQSTGPFQQQMNQPGKAPWGWGGNYGD |
| | | | CAGTRKRMDPWDSTRMFFGWPGSGLRDKGYDASTAQR |
| | | | ANDSIQRVQGSGVPNAYAQWWGLANAAYDRYLGNPIPA |
| | | | PPSPGGSSSGAPALTPNPAWRGDPLFLPQLLRAFGVSVT |
| | | | TYTDADGIRWDERGHGDFGKISWVLWHHTGSVNETDNGI |
| | | | AHHPALGLAANMLIHPDGHVVLTGSGIAWHGGIGVYPGIP |
| | | | EDGINQISIGIECSYGPDRDGRYTLPWPEAQMNAMIAVGG |
| | | | AISWFLGDTLPPSHQIAHKEWAGADNPLGVNKQGKPDPG |
| | | | NLDMTWFRAQIAARAAAGPTTGGDDWMTNPDAITMLAAI |
| | | | YRETVTQKSPSRSFMAEDGTLIDTPLGIDWNTDGNAWTL |
| | | | VMTEAYWNDVPLAITVVEDIAANGVRQTSWAGSADKDDQ |
| | | | VKWNQWLRDFGQAYCAGLVRRKAQWTALVAAVSALAAA |
| | | | QSAAALTAEATTPAAPKKRAPRKRAATKPTTTTTGGAE |
| YP_009964060 | Rem711 | 78 | MPGSEIPRYWPLGAGRIVTSPFGPRSGGFHAGVDFGRNG |
| | | | GSAGMPVYAVQSGTVIYAGAAQGYGGPDPAGWLVIDSTD |
| | | | AEGSGCLEYGHIVREVSKGQHVSAGQRIGHINPDSRTNG |
| | | | GVAPHLHLSDMPREYNPGAKQDPMKRLAGAREPEAAPP |
| | | | APAPNTGGNATVALADPITKALWTARNRYSPRGLPSPMW |
| | | | IGCHTSESRSRAVNLRDYCERNSVSYHRIVDDTDIVGMVR |
| | | | DTDGSWSAVGANKYAYHICWSSSFASWSREQWLDPTPG |
| | | | DGFNERNALRLGAKQIAYWIQQSRAAGRPIPVEWIGGRN |
| | | | RPPWGLNGICGHVDFGAWGGGHSDPGPNFPVDTLLSDV |
| | | | REFLTGEPQPPIVAPPPVGVPGTNPDKYADWMLYQGNPR |
| | | | NNIDRVRAVQRRLKYAYAGYAGHLAIDGDFGPLTRMAVE |
| | | | EFQRRSKLVVDGIVGPMTAAALKP |
| YP_010104319 | Jeeves | 79 | MVTRANVEATKAFIRARLGNPYVYGGALSENVRQGTDCS |
| | | | EVWQTVLEMVHGRWVQGRQSEGATTESYRYIDVGQVGP |
| | | | FGTIRVAHWRDIPADAAARIAFHHGPGGGANSHMWGELD |
| | | | GMRIESGGSKGLQTGDRAMAVESSYATAWAYLPGPIGGV |
| | | | GSLGIGSTGPAVIALQHKLNERGAGLDVDGDYGNLTASAV |
| | | | TRAQQELHVVGDPPGVAGEATQRALGLLGADGAIPAQPN |
| | | | VLAAEYLAQATGLSVTKAQQILPTMQSGLKLADATNVNRIA |
| | | | MFYAQTGHESANFNATEEYASGAAYEGRADLGNTHAGD |
| | | | GVRFKGRTWIQITGRHNYGEFSKWAYAKGLVPDPNYFLD |
| | | | HPQELSDIKWAGIGAAWYWTVARTDINELSDRRDLDTVTR |
| | | | RINGGLNGIDERRTRYNRALGMGDKLLTIFANEPEDEWEA |
| | | | LMADQTRYPSRAWYRSDDVPAHTPLDLLRNIDGMKWDE |
| | | | RVTQAAFRGEVWAIETIAKVAKGEGPEKSPAAIEESKNLV |
| | | | RQLKAALAAQIGDSK |
| AVO21739 | Jeon | 80 | MAVARANVEATKTFIRARLGNPYVYGGALSPTNVKQGTD |
| | | | CSEIWQTTLEMTLGRYVPGRQGEGATTESYRPKSMGGPI |
| | | | PDGGIGPFGTIVTRDRNAVPANAVAKIGLHHGPGGGANS |
| | | | HMWGELDGMGIESRGGGVGVITNPRSMRWDDSYANAFA |
| | | | YLPGPITEDGTPAAPEYVLLGRNYENSGDRVRQLQRALN |
| | | | SYGYGLDEDGEYGPLTEQAVGAFQRSKGLEVDGIAGPVT |
| | | | LAALGLTFGRPVESSPEGLALFRQIMSRTDATNAWLATCL |
| | | | PYYIEAMRAAEINTPLRAAAFASQIGHETSGLKYMAEIQTN |
| | | | GPGWTEDRRIYRGRGAIQLTWSSNYRRFGQWCREQGHV |
| | | | TDPELFVKNPELVEHPQWGFLAASWYWRFGGPKPGQIN |
| | | | SYADKGDILAVSRCVNGWIEGVDPVGWADRRSRYLNCM |
| | | | ALGARIMNLTTVDPLEELLKMKLNSMSIYRTPGEGPIDAAI |
| | | | MLAALDAHGPHEDYVEKMARRGDEDALFRVARVAMGKG |
| | | | AETTPAAIKQATDVLLEIAETNRAAVNAALGKV |
| YP_009949608 | LilMcDreamy | 81 | MLVALGMRLENGWPECDLSDTERLTIPGTPLSLPIRKGQP |
| | | | HAILQAFFRDVNEFIEPANNARGYTDEGSWTENNSVYTSN |
| | | | HKGATAVDWNWSDHPVEVKDGGWNGSVLINGSQVPEM |
| | | | RRLLAFYEGMVYWGNDWTSFIDSMHFQMGYDTCCGGDA |
| | | | AKRVDSFIQRKIRADGFSTYRRGGVPRGGGTAQAPAAPA |
| | | | NPIPPTSGLTGEVLWRIAGRPDRVTVARYAALLPRLIECLH |
| | | | ACECNTIDRRAMWFAQVFHESGGLFYTEEIASGDAYDTR |
| | | | TDLGNTPQVDGDGRLYKGRSFIQVTGKNNYAAMSGWAF |
| | | | EHGYVPTRDFFVVHPDRLDDDEYAFLGVTWYWTTQRPM |
| | | | NTYGDARNIEMGSRAVNGTNPKTGRANGIEDRIAIYNRAL |
| | | | AENANLLDPANVAPLDPWEELMTLKVPSLSIYADPDEPDV |
| | | | PIPVMIAAQDAHGPHEPYVEKQAKLYGDADSIFRIVRTAAG |
| | | | GGRVKTPAAIKQANDVLDEIKANHPEFIVAALREAQKK |
| YP_008430665 | LittleCherry | 82 | MSFRTAYGNAYSENGWRMCNRDECVTVSGPYMNTAPLR |
| | | | RGPAEKLLGEFVRRYHQVCAPVVSPVWGWSETNDVGNS |
| | | | NHLSGTAVDVNAPQWPWGLRKMPADLVARINVLLDQFEG |
| | | | AIYWGRNWNRPDEMHFQLNWREGDAKYDRIIAKFSGGS |
| | | | VPVTPHVPNLPTDDSELLMRGSANVEQTRILQAGLKKVFP |
| | | | SYAGHLEVDGDYGPETEKAVRRFQSGSGLVADGIVGPAT |
| | | | RAELAKYNIILKPAAPQEIIPVSKTPVVVGPADDQLNMRFN |
| | | | CLGGKTLVEAVAEIRDAVCGTNDKDKTGVVLK |
| YP_009950835 | Paola | 83 | MDVTRANVEGAKAFIRARLGAPYQYGGALSSTNVRQGTD |
| | | | CSEVWQTVLESVFGRYVPGRQFEGATTESYRSVKVGEV |
| | | | GPFGTIRVARPQDIPADAVVKLAFHHEGNGGASSHMWGE |
| | | | LDGMLIESASKKGLCTAPAAWPIEHSYANAWAYLPGRIVE |
| | | | DGAAPTVVEPQDTLYADVSEWQVPVTDAYTDAGYRVLCI |
| | | | RSNDGTHRDEDWANNYAWCKRAVDDGRLAFFIVYFVWR |
| | | | PNWQAAVDTLKAQVGEPHPKMAVMLDVESWNGQIGGD |
| | | | QSAGINAAFEQIAGWLGDRRRVIGYGNTGDLNALWPRKP |
| | | | EGVRLVVAGYGKLPTYPGMIAHQYTDGQGYGGGLPEGA |
| | | | PPFGRCDMNAANGLTASAFARALGIEPSTTGEDDFMSAL |
| | | | SADEQREMLTYLRWAFAPGTGEFRKRFPSRSPLRHLGEG |
| | | | VIDTAVGIDLNDDANDHVVLVKELAEIGDPGALALLHEVAG |
| | | | ADPVLYPDRQEDRKLAQRILDHLPQSSTKPAEPDTTPAPV |
| | | | RNVACAQSGAGCILVANGGDGSCALAGDDCVLRKGASA |
| AOQ28958 | Waterfoul | 84 | MDVTRGNVEAAKAFIRARLGNGYVFGGQLSPTNIKQGTD |
| | | | CSEVWQTVLELVFGRYVPGRQFEGATTESYRYVKVGEV |
| | | | GPFGTVRVARPQDIPADAVVKLAFHHEGNGGASSHMWG |
| | | | ELDGMLIESASKKGLCTAPAAWPIEHGYANAWAYLPGRIV |
| | | | EDGTAPTVVEPQDTLYADVSEWQVPVTDAYTDAGYRVLC |
| | | | IRSNDGTHRDEDWANNYAWCKRAVDDGRLAFFIVYFVW |
| | | | RPNWQAAVDTLKAQVGEPHPKMAVMLDVESWNGQIGGD |
| | | | QSAGINAAFEQIAGWLGDRRRVIGYGNTGDLNALWPRKP |
| | | | EGVRLVVAGYGKLPTYPGMIAHQYTDGQGYGGGLPEGA |
| | | | PPFGRCDMNAANGLTASAFARALGIEPSTTGEDDFMSAL |
| | | | SADEQREMLTYLRWAFAPGTGEFRKRFPSRSPLRHLGEG |
| | | | VIDTAVGIDLNDDANDHVVLVKELAEIGDPGALALLHEVAG |
| | | | ADPVLYPDRQEDRKLAQRILDHLPQSSTKPVEPDVTPEPA |
| | | | RNVSCAQGGAGCILVANGGDGACALAGDDCVLRKGVTA |
| YP_008859120 | Zaka | 85 | MALGAPMENGWPECDLSDCDYATVPGTPLRLPFRKGHP |
| | | | FVILQAFLRDLNQFVEPLMNARGATDEGSWTDNNSVYTS |
| | | | NHKGATAFDYNWSDHPMGNALAGWHGSVLISGEQEPAV |
| | | | RDLLRFYTYRGIQLVWWGNDWSSPKDSMHFQMGYSTVN |
| | | | NPAIVQEFIGKFIRPDGYSTYRRGSSAVVPTELTVPLTPAA |
| | | | NGRWTSPSPAWAHLIMRESGGNPTIIQQIHDVNSGGNEA |
| | | | EGLFQITPRTWKAHNGTDFATSARFATPQQQAIVAARIITR |
| | | | NPSGSDWGAGLPGREDAAQLRAGLVPTQTTNDPLEELM |
| | | | AMEVESFSIYATPGEPKIPVHVMIQSLDAHGPHEPYIEEQA |
| | | | RHGDRDAIFRVARTAAGKGKYGDAPGPVKQASRVLKELE |
| | | | EAGVLAEYLKGN |
| AVO22409 | KittenMittens | 86 | MALGGRMENGWPECDLSDCDYATVPGTPLRLPFRKGHP |
| | | | FIILQAFLRDLNEFIEPLMNARGATDEGSWTDNNSVYTSN |
| | | | HKGATAFDYNWSDHPMGNANAGWNGSVLIAGDQVPAVR |
| | | | ELLRFYTYRGIQLVWWGNDWNSPKDSMHFQMGYNTVNN |
| | | | PQIVQEFIDKFIRPDGFSTFRRGGAAPSAPAVDAATILARA |
| | | | TGIAYAKAQEILPTLRDGLILADCKSVPRIAMFVAQTCWES |
| | | | DHYNATEEYANGPLNEERWIYKGRTWIQLTWKSAYAGFG |
| | | | QWCFERGLVNDPNIFVNHPRMLAETRWAGLGAAYYWLT |
| | | | TRRPTRKYPTLNEASDARDVLVATQIINGGTTHLAERQAL |
| | | | YNRAIALGDDLLQILGGDEDEMAGWDQTKVDRAMVLLENI |
| | | | AGVRRKSRSPLRWPYEKEIDTAIGFAWTADANVHVQLVE |
| | | | KLAVIYGDPTSIALLTAVANTDEPGREGDRELARRILARCN |
| | | | EDAVQEAHKRINEWLEAEKAHKVGV |
| QWY84390 | Knocker | 87 | MALGMTLENGWPECDLSDTERLTIPGTPLSLPIRAGQPHA |
| | | | ILQAFFRDMHDFIEPVTNAKGIADEGSWTENNSVYTSNHK |
| | | | GATAVDWNWSDHPVNFMAPDPRAGWNGSVLIPGDQTPA |
| | | | VRELLAWYEGMVYWGNDWSSFRDSMHFQMGYNTCCG |
| | | | GANAARVQSFIDRKIRADGYSTYRRGGVPRGGGPAQAPA |
| | | | APANPIKPTSGLTAAVLHRIAVKRLGNAAPPLSFYEERIGA |
| | | | FLAMLRDCNANTIDRRAMVGAQLFHEAGALRYVREIASGA |
| | | | AYEGRKDLGNVYPGDGTRFRGRGWIQLTGRAHAAGFSG |
| | | | WMFRRGKAPTATYFVDHPEQMETIENAAQVAVYYLTVSR |
| | | | PGFMALADARNIVAATKAINGGTNGLADRQAWYDLFLAEN |
| | | | ANLLDGPETPTDPIEELFTMTAPFRSTSHYRTDDEANLTF |
| | | | GHILRSVDAMTHEQMVELAALRGEVWAIQLLAKLANGQLP |
| | | | GAKDPDGAFWVARAVSLLNAIQTTRPDWITAATEAARGG |
| | | | TA |
| YP_00224 | Konstantine2096 | 88 | MAFIQKQGYWKSENGWRMCDTAELDYTPVPETNFKLGV |
| | | | RKGAPSVILKSLIWRLNRIEPMIVNQIGCYTDENSMRNSNH |
| | | | NSATAIDYNWNLHQYQKWGTWGNNRAAVDKVIADFRGIV |
| | | | EFGGNWTSPRDEMHFELHFAEGHQGTEQLAQELRDGLW |
| | | | GIWKAGTAPSDAPSSGGGSSAGDILRIGSTGPEVLKMQK |
| | | | GMNAVFKNYKAMPLDEDGIFGPMVQAAVIEFQQRSLISVD |
| | | | GEVGPQTKAKLAEYGIVLTGQTTPTSPPAPAAFVYPSHEE |
| | | | MVKQLWEQAFGPQAKGWESLFGKSVDGSRGKFTVEAIA |
| | | | DIRNVATQ |
| YP_009958984 | KristaRAM | 89 | MTTKDQVAQITIAEAKARSYTRSECLAIMSTFYQESGWND |
| | | | TIWDPTHTTYGIAQQDGSYPHRFDGAAAQIKGFFDKLDV |
| | | | WRAKPGASTDIWLNICWMQQAPNWPSADYWYANGRRA |
| | | | YLTEIKSRIATVTPYLDKYWPTTGGTDVPETRPPYNEFPIW |
| | | | SENHYNTKRTVNDIDAFLLHTSEGFVGRDDAAEALSLWY |
| | | | QPRSRQVAYHYAVSQASDGGVTVVDNVDTDYASWSALS |
| | | | ANGRSINLCFAGTRAAWSRNEWLGKFGNAIDVAAYLAVQ |
| | | | DCKKYNIPTKVIAPPYTGRLPGITDHRYVTQILKDGTHTDV |
| | | | GDGFPWDYFAERVNHWAAGGKTEPEPPKVKRFPDDWS |
| | | | DREILVEILRQLRGYNLTGWPQLGGKTLVDAVAELLGH |
| YP_010061435 | Yecey3 | 90 | MVTRANVEATKEFIRARLGNPYVYGGALSENVRQGTDCS |
| | | | EVWQTVLEMVHGRWRQGRQAEGATTESYRPRSMGGPI |
| | | | PDGGVGPFGVVLVDHWSKIPANAAAKICFHHGPGGGANS |
| | | | HMWGELDGMLIESGGSKGLVTGDRAMQINSSYANAWAY |
| | | | LPGPIGGTASLGVGSTGPAVISLQTRLNERGANLEVDGEF |
| | | | GPLTADAVFKFQQNNHVVGDPPGVAGEATLRALGLLGND |
| | | | GAIPAQPDTLAAEYLAAATGLSVARAKQILPAVVDGLKQSS |
| | | | ATNVNRIAMWLAQIGHESAGFDATEEYEKGDGGATERWK |
| | | | YLGRTWIQLTWLSNYLGFSKWAFDRGLVPTPTYFGDNPR |
| | | | ELAELKWAGLGAAYYWTVARPDINALSDRRDLDTVTRRIN |
| | | | GGTNGLTDRRNRYNRALAVGDNLLRIIATTTEPLDEWDAL |
| | | | MADQTRYASRAWFRSDNVANLTPLDLLRNIDGMKYDERV |
| | | | VQAAFRGQVWAINAIAALAQGQGPEGQNPVAVAEAQDIIR |
| | | | QLQAALAAQNGASK |
| QED12198 | Yeet | 91 | MQVLGQEITKVRVAGADAARVRIFGEDAWTAEPPTPVPD |
| | | | PVSYFDWHEGSGSTTTSIVSSHVLTAVNPSTAWDGESAA |
| | | | GQFEGDIGDVATSTWSIAVEFTLSAGSSWRNILHTSGAGA |
| | | | ADEVWLQLNGSTLSIWTSAGASTGFDPAQPTLPPNLPIQL |
| | | | VMTNDGTTQRVYVDGELVSSRARNNILEPTSAVIMDTSEK |
| | | | LNGYVHSLRFWDVVLSDAEVEALMPDSGDEEDPPLFRG |
| | | | MTDEITQYGITWTFDQEYMAGQFITGDWWVVGPVTVTSV |
| | | | SPAPQGRLDGEDDDDLTGEAGQRVDDRMRHGSMVLTDV |
| | | | TTDQAYDSRCRFYNPARAISYPYNLAANRSLISSVSAVHP |
| | | | GGSEMGDLADGAEGRVMETAAVLTCLDEEPPEDAFRPC |
| | | | YWGADKSKIVRASDIQWDRLANVTPASSPPTLVEYARFFE |
| | | | RPWLDHMQAYWVGQFLWPWKNQPGYGRGYAAVVGTA |
| | | | GLLVNCNYTEAQKRPIVHGLIQLGIDLAGMLKADSCFRVS |
| | | | GGHGSGRKFPVVFAHLMLDDDPYFQFEADAVGADVGED |
| | | | GDGEPQALFGEDASTYWGETYYGAPACFQMVGYSSEVP |
| | | | PHQESPPPYTDWDDTSEGYRQCCTVGAWVGQTLAILLM |
| | | | GAKAAWNHDSYFQVVDDWMADPDPYAANRGGHPRPDD |
| | | | WQETNAWHPWATRMWNLHRSSVPAQDDGTDNYKWMA |
| | | | LDREWVPNPPPA |
| QOC58851 | Periodt | 92 | MADRFFPMRDGTYALSSGFGARWGTQHRGLDFAAKDGT |
| | | | PIYAAQAGTVAYIGPAQGFGQWIVIDHPAADGAGTTVYGH |
| | | | MWNAFVTGLKAGDRVQAGQLIAYVGANGQSTGPHLHFE |
| | | | VHPTVWRQGSQIDPLPWLHGSRNPGDAPAPAPADPPAS |
| | | | APPGGQRMQDPFTGEVWSPNRSVRQKPAPRWIAIHTQE |
| | | | GGRTARDLCEGWLAKRESQVSYHVAVDDREILKVVAESD |
| | | | RPWAAANANDYAFHVVAAGSYAGWSRGKWLETDASDG |
| | | | KNEDVELTNLAKVCAWWCQKYAIPAEWIGGRGVPWGRD |
| | | | GICGHEDFGAWGGGHHDPGPNFPDDELIRRVRVLLGGST |
| | | | PEPLPPAPPVALPGTNPDQYAGVLLYRGRPGQDPRQVRV |
| | | | LQTRLKRAYSKLDVDGIFGPHTEACVRDYQHLHPPLVADG |
| | | | IVGPATAAALGLVF |
| ASR86464 | Changeling | 93 | MTFVVTRQRAQEVHDRARARAGLPYAYGGAFTNDPKRS |
| | | | TDCSGLVLQTAAWYGGRTDWVGNRYGSTESFRLDHKIVY |
| | | | DLGFKRLPKGGLAALPFKPVMLVGLQHGGGGVYSHTACT |
| | | | LMTMDIPGGPVVQSARGVDWESRGGNPGVRLYDGARA |
| | | | WNDPLFHDFWYLDARLEDGVTAEVDRAEILARATGLTYN |
| | | | KAAVLLPAVRDGLIQADCTNPNRIAMWLAQIGHEADDFNA |
| | | | TEEYASGDAYDTRTDLGNTPEVDGDGRLYKGRSWVMITG |
| | | | KDNYRDFSRWAHGRGLVPTPDYFVVHPHELAELKWAGIG |
| | | | AAWYWTVERPDINALSDRRDLETVTRRINGGLTNLEDRR |
| | | | RRYNLALAVGDQLLELIGDDDELADPTIQRFIREIHAAMFN |
| | | | TVVTQSPYGDPRNPDGSEPDSNTWQLHELIKNGDGMGH |
| | | | ARYVEESAKAGDLRELARVVRSAKGQGRDKSPEFVARAR |
| | | | NVLAQIEAANPEYLEAYIARNGAL |
| QBP30491 | Charm | 94 | MVTRANVEATKEFIRARLGNPYVYGGALSNNVRQGTDCS |
| | | | EVWQTVLEMAHGRWVPGRQAEGATTESYRGIKPGQVGP |
| | | | FGTIAVAHWRDIPANAAARIAFHHGPGGGANSHMWGELD |
| | | | GMRIESGGSKGLVTGDRALAVESTYATNWAYLPGPIGGT |
| | | | ASLGIGSTGPAVITLQAKLNTRGAGLEVDGEFGPLTAAAVT |
| | | | RAQQELHVVGDPPGIAGEATLRALGILNDNGAIPANPDTL |
| | | | AAEYLAAATGLSVARAQQILPAARDGLLRSDATNVNRIAM |
| | | | WLAQIGHESAGFNATEEYEKGDGGATERWKYLGRTWIQL |
| | | | TWLSNYMGFSKWCFERGLVPTPTYFGDNPRELADLKWA |
| | | | GLGAAYYWTVARPDINALSDRRDLDTVTRRINGGTNGIED |
| | | | RRNRYNRALAVGDNLLRIIANSPEPQDEWEALMADQTRY |
| | | | SSRAWFRSDDVANLTPLDLLRNVDAMKYDERVIQAAFRG |
| | | | QVWAINAIATLAKGEGPEGKNPAAVLEAQDIIRQLQAALAA |
| | | | QNGASK |
| NP_818008 | Che9d | 95 | MDINIMRQALSPTSASDTWLAECLPHMEEAMRAAQINNP |
| | | | RRASAWYSQIGHESAGLRYMAEIKTSDPSWSWDRTRYR |
| | | | GRGPIQLTWQGNYRKFGQWCKDQGYINDPELFVNQPEL |
| | | | VEQPKWGFLAASWYWLCAGPRPGQINGFADVGDIVAVS |
| | | | RCINGWIETKLPNGMPDRQQRWDHCLALGDAIVPGNITPP |
| | | | KEIPVTWTGDPVWLEEVLRPALGDRLKTLPGWQNSGHG |
| | | | DFKDIRGVMVHHTGDSRESAQSIRNGRPDLAGPLANIHIA |
| | | | PDGTVTIVAVGVCWHAGQGSYPWLPTNNANAHMIGIECA |
| | | | WPTPRPDLPKGYDPAERWPDAQIISMRDTCAALALKLELP |
| | | | ATRVIGHKEWAGAAQGKWDPGNLDMSWFRGEVRKDME |
| | | | GFVFPGEHPPIEPQPGPTLPPDYAKETWDQLRIQWPQLG |
| | | | GRTLVDAVAVIGEKLGIEGFYDVKKTQS |
| YP_009203068 | Luchador | 96 | MVTRANVEATKAFIRARLGNPYVYGGALSENVRQGTDCS |
| | | | EVWQTVLEMVHGRWVQGRQSEGATTESYRYIDVGQVGP |
| | | | FGTIRVNHWRDIPADAAARIAFHHGPGGGANSHMWGELD |
| | | | GMRIESGGSKGLVTGDRAMAVESSYATAWAYLPGPIGGT |
| | | | GSLGVGSTGPAVIALQHKLNERGAGLDVDGEFGPLTASA |
| | | | VTRAQQELHVVGDPPGVAGEATLRALGLLGADGAIPAQP |
| | | | NVLAAEYLAEATGVSVQKAQQMLPTMRDGLKQADATNVN |
| | | | RIAMFYAQTGHESANFNATEEYASGAAYEGRADLGNNYP |
| | | | GDGVRFKGRTWIQITGRHNYGEFSKWAHWKGLVPSPSY |
| | | | FLDHPQELSDLKWAGIGAAWYWTVQRPDINALSDRRDLD |
| | | | TVTRRINGGLNGIDERRTRYNRALAMGDKLLTVFASNEPQ |
| | | | DEWDALMADQTRYPSRAWYRSDDVPAHTPLDLLRNIDG |
| | | | MKWDERVTQAAFRGEVWAIEAIAKVAKGEGPEKSPAAIEE |
| | | | SKNLVRQLKAALAAQIGASQ |
| AZS06592 | JacoRen57 | 97 | MAFIKKQGYWVSENGWPMCDTNELDYGAIPDVGMKLGV |
| | | | RIGEPNAILKALICRLHREVEPAIVSQIGCYTATNSMRNSN |
| | | | HNSATAIDFNWGKHPWKVRGTWGALKQKVDRIVADFRG |
| | | | WVEWGGYWSDSYVDEMHFEMHFAPGHQGTIDLARELWS |
| | | | DGLWDIWKPGVRPPSTPAPPTTVYFLLTIGSTGDTVRKLQ |
| | | | AQMKEVFRSYAGHLEVDGIYGPRTAAAVAEFQRRTGIGV |
| | | | DGDVGPITRTTLKKYGVIL |
| YP_009009507 | 32HC | 98 | MPGSEIPRYWPLGAGRIVTSPFGPRSGGFHAGVDFGRNG |
| | | | GSAGMPVYAVQSGTVIYAGAAQGYGGPDPAGWLVIDSTD |
| | | | AEGSGCLEYGHIVREVSKGQHVSAGQRIGHINPDTRTNG |
| | | | GTPSNPIAPHLHLSDMPREYNPGAKQDPMKRLAGAREPE |
| | | | AAPPAPSTGGNATVALADPITKALWTARNRYSPRGLPSP |
| | | | MWIGCHTSESRSRAVNLRDYCERNEVSYHRIVDDVDIVG |
| | | | MVRDTDGSWSAVGANKYAYHICWSSSFASWSREQWLD |
| | | | PTPGDGFNERNALRLGAKQIAYWIQQSRAAGRPIPVEWIG |
| | | | GRNRPPWGLNGICGHVDFGTWGGGHSDPGPNFPVDTLL |
| | | | SDVREFLTGEPQPPIVAPPPVGVPGTNPDKYADWMLYQG |
| | | | NPRNNVDRVRAVQRRLKYAYAAYAGHLAIDGDFGPLTRM |
| | | | AVEEFQRRSKPLVVDGIVGPMTAAALKP |
| AHJ88440 | 40BC | 99 | MTLENGWPECDLSDTERLTIPGTALSLPIRKGQPHAILQAF |
| | | | FRDVNEFIEPVMNARGLSDEGSWTENNSVYTSNHKGATA |
| | | | VDWNWSDHPLGVKNGGWDGSVLINGSQVPAMRELLTW |
| | | | YEGMVFWGNDWSSPVDSMHFQMGYNTCCGGDNAKRV |
| | | | DSFIQRKIRADGFSTFRRGGTPRGGGFAELPAAPVHPIKP |
| | | | TSGLTPEVLWRIAGGAASKLPVSHFERWFDELVECQAAC |
| | | | GVLGNIDRSAMWYAQVFHESGNLVHTEEIASGAAYEGRC |
| | | | EGLGNCQPGDGVRFKGRSFIQVTGRSNYTKLSGWAHSK |
| | | | GYVPTPDYFVVHPDQLDDEQYAMLGVTWYWTTQRRMN |
| | | | DAADARNLELATRYVNGGTNGLDHRRAIYNRALAENANLL |
| | | | LTNPVEPWEELMATAVPSLSIYANPGEADVPLAVMIAALD |
| | | | AHGPHEPYVERQAQEFGDVDSIRRIARTANGQGRVKTPA |
| | | | AIKQATDAFRLIPPEFVRAAIPA |
| YP_008409305 | Adzzy | 100 | MTAIITRQRAQEVHDRARARKGLPYAYGGAFTNDPRRST |
| | | | DCSGLVLQTAAWYMGRTDWVGNRYGSTESFRLDHKIVY |
| | | | DLGFKRLPPGGIAALGFTPVMLVGLQHGGGGMYSHTACT |
| | | | LMTMDIPGGPVKVSKRGVDWESHGNRNGVGVDLYDGAR |
| | | | AWNDPLFHDFWYLDAKLEDAPAPEDDAVEILSQATGLSTE |
| | | | RALEILPAVRDGLIASECVNANRIAMWLAQVGHESASFKY |
| | | | TEEIAKNGRYAPYIGRTWIQITWDYNYRAFSQWCFDRGLV |
| | | | STPDYFVRNYTDLALLKWAGLGAAWYWTVARTDINALSD |
| | | | RRDLETVTLRINGGHNGIADRRERYDRASALGDRLLALISD |
| | | | AQPIDPFEEEMMREVESFSIYATPGEPKIPLFVMLQSLDAH |
| | | | GPHEPYVEEQARHGDRDAISRVIRTAAGKGKYGTASGPV |
| | | | NQASRVLAELEDSGVLSNYLKGN |
| AYQ98609 | Riparian | 101 | MITENGWPDCGPNDLDRKPIPGTDVVIPLQKGQPSLILKAF |
| | | | AADLNWYVESVYNSRGGTDEGGWTGTNSVPTSNHLGGT |
| | | | AFDYNWSDHPLGYAAPDPRAGWNGSTIIKGDQTPYVREL |
| | | | LDYYEGMVYWGNDWRSPKDSMHFQMGYNTYGPVGSTT |
| | | | WKRVEDFIARKIDITTGFSRFREHKGTTGVNFTALLSEAML |
| | | | DVSGVDYAYYVTLVARTLRDCECTTEARIAMWCTQVGHE |
| | | | SLSLKYMEEIADGSQYEGRTDLGNTQPGDGKRFKGRGPI |
| | | | QVTGRYNYTKFSEWAHSKGIVSTPDYFVMNPSELSKPEY |
| | | | GFHAVTWYWTVQRPMNQAADARNLEQATKYINGGLNGL |
| | | | PDRKMRYERTTAMGSRLLNLLLGEDDWMSDPTIVKMIKEI |
| | | | HGCLFNQIGSQSRYRADGEGDIWKLHELIKNDDGMVHEA |
| | | | HIERLAVLGDPSSIALVARNSERGDKLAQAVLIRIEDKYLTE |
| | | | TQQHLKNTIIAQRFPEG |
| YP_010013125 | Tourach | 102 | MAFVSRYGNWYSENGWRMCDANELDRGAVPGTSLVLPI |
| | | | RKGVANLILKGWVAWFHRNVESLNNARGFSDEGAWTPT |
| | | | NDVANSNHLSGTAVDLNWSDHAFRVSYSGFTQAEINKVR |
| | | | EGLKLFEGTIWWGQDWNSPKDPMHFQLNYGEGDARNAA |
| | | | FATKLQNGYLNIWSGGGGVIEPPAGVDDIYAELGDSGDRV |
| | | | KRLQQFFNDNFKSYSRLEVDGDFGPATKAVVVEFQKRVG |
| | | | VLADGIVGPVTLAAMVKHGFKTESGVKVANLPEDWTDRQ |
| | | | VINDIWEQLRGPDGKGWERLGKNAKGENLSLVDAIAEVKK |
| | | | RLEAA |
| YP_009961543 | Pollywog | 103 | MSFTWFADKPLRTREQIAREVHAVSLARGLDELATVIALM |
| | | | TISTEVGTGTGDDRKWWCPANDRVPATKNYPHDSRSDD |
| | | | NRSSGYFQQQPGPNGEPWWGTPENMMTLPQAANTFLE |
| | | | RLSDDYRRAANNPRLAGEFAQRVQQSAYPDRYADKWDE |
| | | | AWTVLRRALGSQPPATPEVPMPENRPDFNEFAIWSANNS |
| | | | VRSGKPTMFLIHTQEGGGGDAAAENLAKWFQNSNGVSY |
| | | | HYTISQASDGGVTVVDCVDTDRAAWSVGNANSISINLCFA |
| | | | GSRAAWSRDQWMKQSNAIDVAAYLAVQDAKKYGFAPLV |
| | | | VPPPYTNGRPGISDHRWVTDVFKWGTHTDVGAWFPWDY |
| | | | FAERVAFWANGGVATEPEPPKVKRFPDDWTDRELAVETL |
| | | | RQQRGYTLNGWPQLGGRTVVDVLGAIGAKLGVEGCYDV |
| | | | KGKS |
| USL89142 | Poompha | 104 | MALGGRMENGWPECDLSDCDYATVPGTPLRLPFRKGHP |
| | | | FIILQAFLRDLNEFIEPLMNARGATDEGSWTDNNSVYTSN |
| | | | HKGATAFDYNWSDHPMGNANAGWNGSVLIAGDQVPAVR |
| | | | ELLRFYTYRGIQLVWWGNDWNSPKDSMHFQMGYNTVNN |
| | | | PQIVQEFIDKFIRPDGFSTFRRGGAAPSAPVVDAATILARA |
| | | | TGIAYAKAQEILPTLRDGLILADCKSVPRIAMFVAQTCWES |
| | | | DHYNATEEYANGPLNEERWIYKGRTWIQLTWKSAYAGFG |
| | | | QWCFERGLVNDPNIFVNHPRMLAETRWAGLGAAYYWLT |
| | | | TRRPTRKYPTLNQASDARDVLVATQIINGGTTHLAERQAL |
| | | | YNRAIALGDDLLLILGGDEDEMAGWDQTKVDRAMVLLENI |
| | | | AGVRRKSRSPLRWPYEKEIDTAIGFAWTADANVHVQLVE |
| | | | KLAVIYGDPTSIALLTAVANTDEPGREGDRELARRILARCS |
| | | | EDAVQEAHKRINEWLEAEKAHKVGV |
| | Lilbit | 105 | MSFIRSAFADKPLLTREQWMKLFIRVADELDMPDRRGAAV |
| | | | LAAMCAFQEAGADPHNTGKRQIWVPGNNADPCFKANPA |
| | | | AYPHDSMGDDGQSTGPFQQQMNKPGTTPWGWGGNYG |
| | | | DPEGTRKRMDPYESTKMFMGHPSSGLKKKGYDASNAQT |
| | | | ANDAIQRVQGSGVPWAYAQWWDEANRLYDEVVGDVPP |
| | | | STGGGKVPVSGDPVWLEEVLRKRLGDRLVVHDGWKEYG |
| | | | TGGVMGEIWGVMIHHTGNRNASWESIRNGRPDLRGPLS |
| | | | QALIAPDGTFHLVAVGPCNHAGVGSYPGLGSDGNRRAIG |
| | | | FECAWPTIRPDGSFDKGERWPDAQILTMRDATRAVLEHL |
| | | | GHDHTHVCGHKEFNKVDGKWDPGNMDMNWFRGEVRKA |
| | | | LAGEFDPRAPEPEPQLPSQPGIPTDHDLLVYDQICGRWE |
| | | | MLGWRTPVEALATILDELRGTKNAGSRGFTRGPHDIEDKK |
| MK524490 | Donny | 106 | MTERVLPFDRKIIRQDTGYWCGPASTQMALSARGKYVDE |
| | | | ATLARECKTTVNGTDNVGLIERVLDVRLPEGNYTSMYPG |
| | | | GVKIGSPARPAAERKTRFWWDIVRSIDNGFAVILNWVVPP |
| | | | ARKPIRAVKGSTNPSYGGGTTYHYVTAVGWSDEGNNGR |
| | | | PAVLIADSGFAPNVYWVDLDTAFALIHTDLWKGYAYADLP |
| | | | LIAPPPPGVEVPPGIPVKPGTPPVAVTPPVPAPPTQPPPTK |
| | | | LGSLTDPFTGALWSPNHYDGRGGLGTPGWIAVHTQEGG |
| | | | RTARDLALFLANPANEVSYHSVNDDIEVLKCVAETDAPWS |
| | | | ASNANKYAFHHCFAGSYAGWSRDKWLSPDASDGKNEDV |
| | | | QLTKGAHVVAWWCDKYGIPAEWIGGRAQPPWGARGILG |
| | | | HVDLGQWGGGHFDPGGNFPVNEFIRRVVTFLTGEVQPPL |
| | | | EPLPPVVQPGTNPDAYSDWMLVRGDPRNDVDRVMRVQS |
| | | | KLKRAYAAYAGHLDVDGIFGPATQAAVREFQRRSNLVAD |
| | | | GIVGPMTAAALRP |
| | Heftyboy | 107 | MDVKRANVEGAKAFIRARLGAPYQYGGALSSTNVRQGTD |
| | | | CSEVWQTVLESVFGRYVPGRQSEGATTESYRSVKVGEV |
| | | | GPFGTIRVARPQDIPADAVVKLAFHHEGSGGASSHMWGE |
| | | | LDGMLIESASKKGLCTAPAAWPIDHSYANAWAYLPGRIFE |
| | | | DGTAPTVVEPQDTLYADVSEWQVPVTDAYTDAGYRVLCI |
| | | | RSNDGTHRDEDWSNNYAWCKRAVDDGRMAFFIVYFVW |
| | | | RPNWQAAVDTLKTQVGEPHPKMAVMLDVESWNGQIGGD |
| | | | QSAGINAAFEQIAGWLGDRRRVIGYGNTGDLNALWPRKP |
| | | | EGVRLVVAGYGKLPTYPGMIAHQYTDGQGYGGGLPEGA |
| | | | PPFGRCDMNAANGLTASAFARALGIEPSTTGEDDFMSAL |
| | | | SADEQREMLTYLRWAFAPGTGEFRKRFPSRSPLRHLGEG |
| | | | LIDTAVGIDLNDDANDHVVLVKELAEIGDPGALALLHEVAG |
| | | | ADPVLYPDRQEDRKLAQRILDHLPQSSTKPAEPDVTPAPV |
| | | | RNVSCAQSGAGCILVANGGDGSCALAGDDCVLRKGATA |
| | Gravaillia | 108 | MRFYPLGTGRIVTSAFGATTGRSAPHGGVDFGRAGGSAG |
| | | | MPVYAIQSGTVIYAGAASGYGGPDPAGWLVVDSDDDEG |
| | | | GGCLEYGHIVREVNRGDHVTAGQRIGHINPNNRTNGGVA |
| | | | PHLHVTDWPHAYGQGSRQNVMARLSGAREPEAQAAPTP |
| | | | TEQGSVVGDPVWLADVVRPTVAKFAEYPNWRNRGHGDF |
| | | | KDIRGVMVHHTGGPASAASIANGRPDLAGPLAQLHISRDG |
| | | | TVTVVAAGVAWHAGAGSYPWLPTNMGNWHLIGIECEWP |
| | | | YGEPGINERNAHTVRWRDPEIIALRNTCAAILLRLGLGVDR |
| | | | LIGHKDYAGRAQGKWDPGNMSMDWLRGEVRKDMDGFV |
| | | | FPGEDIVNVTPPPVPAPVPAPPATANVLLHRGMSGPNVVK |
| | | | LQTTLRRWYSKLAVDGDFGPHTEACVRDRQRIYGLDVDG |
| | | | IVGPLTAAKIGLVL |
| | Dewey | 109 | MADPALVSSRPVRFYPLGTGRIVTSAFGSTTGRSAPHGG |
| | | | VDFGRAGGSAGMPVYAIQSGTVIYAGAASGYGGPDPAG |
| | | | WLVIDSDDAEGGGCLEYGHIVREVNRGDHVTAGQRIGHIN |
| | | | PNNRTNGGVAPHLHVTDWPHAYGQGSRQNVMARLSGA |
| | | | REPEAQAAPTPTEQGTVVGDPVWLADVVRPTVAKFAEYP |
| | | | GWRNRGHGDFKDIRGVMVHHTGGPASAASIANGRPDLA |
| | | | GPLAQLHISRDGTVTVVAVGVAWHAGAGSYPWLPTNMG |
| | | | NWHLIGIECEWPYGEPGINERNAYTVRWRDPEIIALRNTC |
| | | | AAILLRLGLGVDRLIGHKDYAGRAQGKWDPGNMSMDWL |
| | | | RGEVRKDMDGFVFPGEDIVNVTPPPVPAPVPAPPATANV |
| | | | LLHRGMSGPNVVKLQTTLRRWYSKLAVDGDFGPHTEACV |
| | | | RDRQRIYGLDVDGIVGPLTAAKIGLVL |
| | Clarkson | 110 | MSFIRSAFADKPLLTREQWMKLFIRVADELDMPDRRGAAV |
| | | | LAAMCAFQEAGADPHNTGKRQIWVPGNNADPCFKANPA |
| | | | AYPHDSMGDDGQSTGPFQQQMNKPGTTPWGWGGNYG |
| | | | DPEGTRKRMDPYESTKMFMGHPSSGLKKKGYDASNAQT |
| | | | ANDAIQRVQGSGVPWAYAQWWDEANRLYDEVVGDVPP |
| | | | STGGGKVPVSGDPVWLEEVLRKRLGDRLVVHDGWKEYG |
| | | | TGGVMGEIWGVMIHHTGNRNASWESIRNGRPDLRGPLS |
| | | | QALIAPDGTFHLVAVGPCNHAGVGSYPGLGSDGNRRAIG |
| | | | FECAWPTIRPDGSFDKGERWPDAQILTMRDATRAVLEHL |
| | | | GHDHTHVCGHKEFNKVDGKWDPGNMDMNWFRGEVRKA |
| | | | LAGEFDPKAPEPEPQLPSQPGIPTDHDLLVYDQICGRWE |
| | | | MLGWRTPVEALATILDELRGTKNAGSRGFTRGPHDIEDKK |
| QFG14375.1 | Starlord | 111 | MTFSPLISGTINHNNKYSSRQGRPVLKIVQHHWAGTGGGI |
| | | | ERMEGPDQASCTYVILTDGRILGHVPEEFRPWTSGSFEA |
| | | | DGDAITIEVQNINGRMDGNDNSPNSWQISDAAYASIIKLIA |
| | | | DVARRYGWGGVAVGNYRGHREFSSTACPGGFLWARMD |
| | | | NTRAQAHAAFVGGKPAEAPAPVAPTTSGKTVWQLADEVL |
| | | | AGLHGSGDARKNSLGAQYDAVQAEVNRRFGVGLPATKA |
| | | | PKSVSQLADEVLAGKHGSGDARKASLGSRYAEVQNEINR |
| | | | RLGGGGVAPQGVNIGALADAVLRGEFGSGDDRKAKLGV |
| | | | NYDAVQAEVNRRLGGGVAAAPPARTVNIGALADAVIRGE |
| | | | YGSGEDRKKRLGSNYAAVQAEVNRRFK |
| QDH48282.1 | Luker | 112 | MNGAQVIGAIAKRNVELGGRSVYTRGWETRGNGQSPAY |
| | | | QGLLLHHTAGGRNVNIDQILIDGRWDLPGPLCNFCIMYDG |
| | | | DLGVISANPANHAGASGGWDTAPFARTGLFNRQIIGCEIQ |
| | | | YPGVEPMSNAQYETAKRLSIATLEVLGKPRDTRWIKFHQG |
| | | | TSIEGKWDPGYANGKTYNISEFRKDVLGMRPTGEDENSA |
| | | | AWLDNYRQLFGYPE |
| AYN58853.1 | Richie | 113 | MVAPVIKMPNTEKHSGVSGIPTQILVLHSGECPLRGGYAQ |
| | | | SLTQWGNVPLAQGGPEASWHWFVDPIAIVSMVDPQFAA |
| | | | WHASEANPMSEGFEQGGYARFTRAEWLTPEGVKQMDN |
| | | | LAWIMAQRAKANGIPAVWLTTDQVTAVTTFGNRTIKGFCL |
| | | | HRQIDPETRTDPGDGYPYDLLMAKVKAYMSGSTFTPQST |
| | | | STAKPQGLFMALTAAQEKTILTRNEKYVDAPISQVDEKAAT |
| | | | AVWETKVDRGAKSVSALQELADCKSMLLAQQGVLAGLTN |
| | | | AIQQLATKPGSPVDLDAIGKAAEAGAAAALADLTATVTIKQ |
| | | | EATNG |
| YP_009885103.1 | Mufasa8 | 114 | MRPVAAEFETSQRFGEGATQGVVANSDPNSGMGYYVWL |
| | | | YGNYQPFGHAGEDEACPIGTPVHAIADGTVVWAGWAED |
| | | | MAGDDSDWGYRQRFYVYKRFPGILTVIRHDNRADGLLYT |
| | | | LYGHLSNNDPAPAGTVVREGDLIAYSGNTKSKTETLEPHL |
| | | | HVEALVDMSYRSGGGLIYGRVDPRQFFGTTTPTTHTADE |
| | | | LFLLDLGLSIP |
| QIN94318.1 | BlueFeather | 115 | MTALVRPHKGPHTQRFGNLQPDGQPHAGDDFGYTDGVN |
| | | | VYPEVYAAAAGEVIYAGDARALGWPNPFYLNPDFDRTDA |
| | | | QDSSAGNVVVLDHGDGMTTYNHLESIQVRKGARVAQGQ |
| | | | RIATTGNTGFSFGKHLHFEWIPYPANFGTATYGRARPTFV |
| | | | TVTAQSTKPKELFTVSQYAALNAKLDTLIEATKPINTASGK |
| | | | KTLRDFVAAGTRAAEAARDNTAPINVTGGQESLRAFVAKG |
| | | | TRAAQANAARLAALEAALKAVLENPSGVSLEAVTDAAAAG |
| | | | AERALANLTGTIELESKNG |
| QGJ92791.1 | Beagle | 116 | MSTGFTLPTSRPVTQPWAAEFDDWDGDGIVDIPGGFYHS |
| | | | IGWFGHNGIDFGCFEGDPVEAVADGTVIFAGDAANHYLLS |
| | | | GGGNVAFIHHEFYGVWTESLHMSRVIVANGQRVTKGQVI |
| | | | GYAGSTGSSTASHLHLGMFASEFPNQWDGWRGRIDPTP |
| | | | YLYGALNSDYAIKTHTAQIKEAEMPAVLHKVVSPAMNRRL |
| | | | AKGKAAVITTDATNTAHQNFAVGGVGTYDIQAYVAGTGLP |
| | | | DGQRITGRFLLVEKGKAPSGYYPFQIDGTFDGKFNGLIGG |
| | | | RFKVNAGTVIWIELTSTVDTSYVASVDGSVLVHPFK |
| QDH92682.1 | Charming | 117 | MTTVIDTAGGFPSPEAIKAAGHSGLMAYVSLSRPGSNFAG |
| | | | KPITRAIADRYRAAGVDIGAIFQYGKPQGTAPSDWTTGFE |
| | | | GGKRMAAQALRIARDAGMPGWCPIYFAVDENITLQQWND |
| | | | TAVHFFRGAGEAIGVEWVGIYGHSRVCHWAIEDKVVGRS |
| | | | PKPGSGPWAWVTRAWSDDTGTGYASLYQRVIDTPSNPG |
| | | | PLVDGIRVDVNDVYAADWGQWSVDRTPNAGQPAPAPTA |
| | | | PGGPADMARIGYGVTHTIPAGSDGPRRADDYVGVHTQEG |
| | | | GKGDAIGLANYCKTAGVSYNDAVDDVYTVRMMPPGNAP |
| | | | WAAVQANAVGYHVVLAGSFVSWSRDRWLSKDASDGLDE |
| | | | DAMLTRAARCVAAACQEFGIPVEWVGNNGATGWPQKRG |
| | | | ICGHKDFGARGGGHTDPYPNFPIDEFMRRVRAFFAPPSP |
| | | | NLIDAEAKVAARWIGKRITGAATPTTTDDETPLFLDGKKIG |
| | | | AFARFESGHVYWRLGAALAYAIPAGGLFEAYAARDWERG |
| | | | LGFPVLRHTVVTTPASKVTAGVQSFEGGVLFTPVGGPVE |
| | | | GFVVHGEIGKRYAAMDWEQGPLGLPTSDERPVPNTDLIE |
| | | | QRFEFGKLTYVPTGVLVELVTN |
| QLF83829.1 | Moosehead | 118 | MADPVWLPDVLRAEGLRCDIYPGAFERGHGDFGVIWGPF |
| | | | MHHTGSFGETPRGIAQHSSLGLASQLHLAANGVVTLCGV |
| | | | GVAWHAGTGSWSGIPKNNGNAVTIGIEAAHNGTAAWSDA |
| | | | QYGAYLKVVRAINKRLGNPWNKVVAHKEYGAIQGKWDPG |
| | | | NLDMKLFRQRLLQAPDKPLVVMNMIELEAKENPWVGVRK |
| | | | AKAGADGERSIGRDGKGRFVEYDNAHIYFHPATGAHAIPH |
| | | | GGLFEAYTERKWETGELGFPVRDFTKLADGAVMAFQGG |
| | | | VLYRKDGMGHHVVKGVIGQRWALEGYEKGPLGWPTSDE |
| | | | IPNGTGGKYQTFEHGVLEWDPSGAVKKIGDAAKDLTLVNA |
| | | | AGIPLAVEAVDLIAA |
| QFG08262.1 | Calix | 119 | MIMTDLANHPGFWLRDDAAKAFDAWEAKYGKRKLNSAG |
| | | | RTKASQQGLINRWHQGGPANRPPYLYRPAEPAETSPHVI |
| | | | DGGIAVDLEDWREAKKTSEEFGFHWYGDGDVVHFNFRG |
| | | | WDGNSAKRGGYQDGTVELKRFQEKLIRMGHDLGPTGAD |
| | | | AVFGPRTKAATLHEQGMAEKNGYPGGKVSDDGLPGPQT |
| | | | EAYLDWWLVGRHAARPSSKSAGELTYADIQAALRRHGYD |
| | | | LVVDNVWGPKSSAALADFQRKSGLKVDRLVGPLTWDKLN |
| | | | R |
| | Abigail | 120 | MAYNYVWIDGHRVEVAVAYAFRRLKAAFEQAFPGLTLRV |
| | | | RSGVRTREEQERLYYGWIHRLPGFNLAAPPGKSNHEESG |
| | | | PIGPRAIDIYDTGGDAGVTTMNTRRDRWMAANAPNYGFS |
| | | | NAGYNFWPREAWHKEFTGKLGVVPPNWVGPTPYPVSLK |
| | | | EDDMPVQIRNVETKMIAMVAPKFFAYIRDRETTEVTKNVF |
| | | | SVVDEQHQLSNADFFRVTRTMGIPDAEVREGNLWYGAPT |
| | | | QPVAVASPPLIVKKV |
| QGH76581.1 | Antares | 121 | MAEYVYINGARLTAWMAYILGVLSNDLYRTFGVWLVITSAI |
| | | | RTYAEQERIFRERYVTAGNINGRRVYDTRVWNGVRWYRI |
| | | | SSAGTVAVPGTSNHEIQGTKAAVDLADTGRDGGVATAGS |
| | | | VRSNWLRARAHLYGMVASGFGFGEAWHYDILNIFQTPPS |
| | | | SGAGNVTPTPKEDDMTVAILLNGRHYYTAGEEFLSHNGS |
| | | | KGQADITRQVNSAQDELHKLTTAQFYDYLDGMGIPRSVV |
| | | | DVNGGGVLNPQSGKIEGNGVWSRRREAVALAEQYAASL |
| | | | AEFVKRNDAAMAALSKAVAALPKAPAS |
| QIG59054.1 | RubyRalph | 122 | MGYLAPANVPISDTRAGHIRRGSKEPGTDYATPYGTDLIA |
| | | | PGDGVILSVDPNPGGAEGRRISFLMDDTGEVIDWIHLSEIM |
| | | | MRAGQRIGRGTKGIAKSGASGFGKDWYYGPHVHVTRRA |
| | | | RQGLPYSQTLDFQAAVEGGGGGGSAPASADVAKGQRTA |
| | | | GGNGVRRRKQPTSKSAENGEMLKPGTVGNFTGWIRGEK |
| | | | VDGIDTWFQGISGDWFWAGGFTQGANTTGLKDLNPKTPA |
| | | | PKPDPKPTSTQRKVGPNGARRRESPSSKAAEAGEMLAP |
| | | | GTIGNFNGWARGEKVEGNDVWFRGTSGDWFWSGGFEG |
| | | | GPVTAGLPEISIAPPVTPPKPVDPKPTPNADNPRNLPVYKP |
| | | | VYPGAKIGLVAPLTMPRGEKGIPPVKVENKIDIVQEHWTG |
| | | | VTADQLDWFSTDNSRGSCPNWFIRPDGTVYELIRPGMKP |
| | | | ALAGPEWNWRSLGWEIQMVEGGKGTPEQFEAVCQLLAW |
| | | | IHSYDGKVLDGTPVTFPLDRDHFKAHRELVATECPGDWW |
| | | | FSQMDAQLARARVIYAEKYAPEQPEEPDTGDLLLARAKAL |
| | | | IEQNNALLEEIVAWAEGA |
| QRI44947.1 | BlueRugrat | 123 | MADVKGGYLRPTPAGGISSSWQAHKDRRPPAFPGPSQE |
| | | | PGTDIACAYGTTLVAPEDGVVVEIKTSNSGATGRFITIDLN |
| | | | DGRRVRYLHLSAVLVTSGKRVTRGQAVARSGASANGRD |
| | | | WGVGAHVHVTLWDFHRYVFGPNGTMDFMPQIGPDNDG |
| | | | GAPGLSYSQEVANQQAWLNQARGEKLVIDGYLGGMTRA |
| | | | AIARYQTFLGVKSDGIWGANTQAAHQKYYDKVTAPAPTQ |
| | | | GTLVTMDHIGTIGNIEGLQKIARLYGYKGRIDNIWGPGTRQ |
| | | | GLANFLNQNYGGRLTNWLRAKWGYTDADDYWGPNMKA |
| | | | ALQRANAANKAAL |
| YP_009847665.1 | Tiny Timothy | 124 | MGYLVPASAPISCSWQCHRDRPKPSTEPGTDHATAYGTS |
| | | | IRAAGAGTIVDSSTSTSGGTGRYVTIDLDDGRRVRYLHLS |
| | | | QVLKRSGRVNAGDIIAKSGASGFGKEWGYGAHVHTTLFP |
| | | | KHAYVFGTNATLDFMKYVGNSTPVTRDQTVANQQNFLNV |
| | | | AQGEKLVEDGILGGETKAAIGRYQTYLRGRGWYNGESDG |
| | | | IWGAGTQAGHEKRYAEWVAATQAPPSPQYHNVTLDDIAS |
| | | | IGNVEGLQKIARLYVPQKVDNQWGPNSKKGLQQFLNVNY |
| | | | GGSLVNWLRSKWGYVGNDQFGPVMKAALQRANDANKR |
| | | | AL |
| QJD52659.1 | AN3 | 125 | MTAVVTRRQAQWVHDMARARNGLPYAYGGAFTNDPKRS |
| | | | TDCSGLVLQTAAWYMGRTDWVGNRYGSTESFRLDHKIV |
| | | | YDLGFRRMPPGGPAALPFKPVMLVGLMHGGGGENSHTA |
| | | | CTLMTMDIPGGPVKMSDRGVDWESHGNRNGVGVDLYDN |
| | | | ARAWNDPLFHDFWYLDAKLEDAPAAPGEMTVPLIAKANG |
| | | | RWGSSSPAWDHLIMRESSGDPTIIQQIIDVNSGGNEAEGL |
| | | | FQITPKTWRAHNGTQFATSARFATPQQQAIVAARIFTKNP |
| | | | TGSDWGAGLPGREDPKQLAAGLVPTEPIGEDGFLSALND |
| | | | AEQREVLNLLRWIAAPEYGELRKLFQNEDMYRTDNVRRR |
| | | | TAVGVALDARTFSWEDRVEKSAERGEQWAIDLVVKAARG |
| | | | ESAGVIRPGSNTPDPFLVNHAKQVLADVERINPEALKIYLR |
| | | | GGSA |
| QFG14375.1 | PinkCoffee | 126 | MTKIITPDKVREVDAMARARDGLYYGYGEAFTRNPKQST |
| | | | DCSGLVLQTGAWFAGRTDWVGNRYGSTESFRLDYKIVYD |
| | | | LWFKRMPRGGLSALPFKPIMLVGLQHGGGGIYSHTACTL |
| | | | MGMDRPGGEVKQSARGVDWESQGNGVFYYEGARAWN |
| | | | DSLFHDFWYLDAKLEIAPPVNEINAEYDRAKGWIGKRIDVN |
| | | | EQPAPDGEGKFIRCERGHIYFHPKVNTGAPAGMRAIAIPA |
| | | | DIFEVWKGQGFERGPLGYPTVRHYTDTGVGTIQAFQGGA |
| | | | IYRKYGTSGGVVVGDIGRRYAALKAEKGPWGYPLGSEKF |
| | | | RNGGQRVQTFEHYDAYWHPSKVIDFLRDPEAPNQ |
| AAG48318.1 | Mycobacteriophage | 127 | MTTKDQVAQITIAEAKARGYTRSECLAIMSTFYQESGWND |
| | Ms6 | | TIWDPTHTTYGIAQQDGSYPHRFDGAAAQIKGFFDKLDV |
| | | | WRAKPGASTDIWLNICWMQQAPNWPSADYWYANGRRA |
| | | | YLTEIKSRIATVTPYLDKYWPADGGTAVPDEPRPDFNEFPI |
| | | | WSNNNSARSGKPTMFLIHTQEGGGGDAAAENLAKWFQN |
| | | | GNGVSYHYTISQASDGGVTVVDCVDTDRAAWSVGNANSI |
| | | | SINLCFAGSRASWMRDQWMKQSNAIDVAAYLAVQDAKKY |
| | | | GFTPLVVPPPYTNGRPGISDHRWVTDVFKWGTHTDVGD |
| | | | WFPWDYFAERVNHWANGGKTEPEPPKVKRFPDDWSDR |
| | | | EILVEILRQLRGYNLTGWPQLGGKTLVDAVAELLGH |
| AAG48318.1 | Mycobacteriophage | 128 | MTTKDQVAQITIAEAKARGYTRSECLAIMSTFYQESGWND |
| (6X- | Ms6 | | TIWDPTHTTYGIAQQDGSYPHRFDGAAAQIKGFFDKLDV |
| His) | | | WRAKPGASTDIWLNICWMQQAPNWPSADYWYANGRRA |
| | | | YLTEIKSRIATVTPYLDKYWPADGGTAVPDEPRPDFNEFPI |
| | | | WSNNNSARSGKPTMFLIHTQEGGGGDAAAENLAKWFQN |
| | | | GNGVSYHYTISQASDGGVTVVDCVDTDRAAWSVGNANSI |
| | | | SINLCFAGSRASWMRDQWMKQSNAIDVAAYLAVQDAKKY |
| | | | GFTPLVVPPPYTNGRPGISDHRWVTDVFKWGTHTDVGD |
| | | | WFPWDYFAERVNHWANGGKTEPEPPKVKRFPDDWSDR |
| | | | EILVEILRQLRGYNLTGWPQLGGKTLVDAVAELLGHHHHH |
| | | | HH |
| BCI54763 | Mycolicibacterium | 129 | MGAAVLAVLMAAALGAAPNAPAYSRDGLPIETLEVPSPAM |
| | litorale | | GRNIRVQFQGGGPHSVYLLDGLRAQEDANGWDINTAAFE |
| | | | WFYESGVSVVMPVGGQSSFYADWYQPAANNSGAVTYK |
| | | | WETFLTQELPAWLAANRGQDPRGNAVVGLSMSGGAALN |
| | | | LATWYPTQFIFASSLSGYLNPSAGLWPTLIGFAMRDAGGF |
| | | | NPQHMWGPTSDVAWRRNDPMVNVGRLAANGTALWIYC |
| | | | GGGVASELDTGHDFGGNFSAGYLENITLSTNKEFQQKYL |
| | | | AAGGHNAVFNFPPKGTHSWGYWGAQLQAMKPDLLRVLG |
| | | | VGVAPPPPPPPALPVPAPRRTATARAAGSLTPRGRIDERP |
| | | | SNRGCRGCGRDRSGRAVRLSRGHPTRRGRRPHRPRQG |
| | | | RHHVGGVHHHPRRAADHTAGPGTPDAPGLQQRLAHPSR |
| | | | PRILGDHRRDGPVADRRRLHRRSLRCGSVRVHPRRAGA |
| | | | PRRQRCAPRRRRRRHLHRARGRTRARRLTRRVRQIAAW |
| | | | TGDPVWLADVLRAEGLTVVEHPGWLHRGHGDFRDIRGV |
| | | | MVHHTGADHATAASIADGRPELPGPLAQVHIALDGTVTTV |
| | | | AAGVAWHAGAGSHPDLPTDRANWHTIGVECANSGTGPT |
| | | | APHRANWPDAQYAALVGTCAAVSRRLGVGAAGIVGHRE |
| | | | YAGRAQGKWDPGAIDVDLLRRDVAACVPLAPGCRSEHV |
| | | | RRLQRRLRDAYRGYAGHLAVDGSYGPQTEAAVREFQLR |
| | | | TPGLRADGVVGPATAVALRLAGG |
| BCI54763 | Mycolicibacterium | 130 | MGAAVLAVLMAAALGAAPNAPAYSRDGLPIETLEVPSPAM |
| (6X-His) | litorale | | GRNIRVQFQGGGPHSVYLLDGLRAQEDANGWDINTAAFE |
| | | | WFYESGVSVVMPVGGQSSFYADWYQPAANNSGAVTYK |
| | | | WETFLTQELPAWLAANRGQDPRGNAVVGLSMSGGAALN |
| | | | LATWYPTQFIFASSLSGYLNPSAGLWPTLIGFAMRDAGGF |
| | | | NPQHMWGPTSDVAWRRNDPMVNVGRLAANGTALWIYC |
| | | | GGGVASELDTGHDFGGNFSAGYLENITLSTNKEFQQKYL |
| | | | AAGGHNAVFNFPPKGTHSWGYWGAQLQAMKPDLLRVLG |
| | | | VGVAPPPPPPPALPVPAPRRTATARAAGSLTPRGRIDERP |
| | | | SNRGCRGCGRDRSGRAVRLSRGHPTRRGRRPHRPRQG |
| | | | RHHVGGVHHHPRRAADHTAGPGTPDAPGLQQRLAHPSR |
| | | | PRILGDHRRDGPVADRRRLHRRSLRCGSVRVHPRRAGA |
| | | | PRRQRCAPRRRRRRHLHRARGRTRARRLTRRVRQIAAW |
| | | | TGDPVWLADVLRAEGLTVVEHPGWLHRGHGDFRDIRGV |
| | | | MVHHTGADHATAASIADGRPELPGPLAQVHIALDGTVTTV |
| | | | AAGVAWHAGAGSHPDLPTDRANWHTIGVECANSGTGPT |
| | | | APHRANWPDAQYAALVGTCAAVSRRLGVGAAGIVGHRE |
| | | | YAGRAQGKWDPGAIDVDLLRRDVAACVPLAPGCRSEHV |
| | | | RRLQRRLRDAYRGYAGHLAVDGSYGPQTEAAVREFQLR |
| | | | TPGLRADGVVGPATAVALRLAGGHHHHHH |
| OCB59153 | Mycobacterium | 131 | MTENGWPACGPELLDRSAVPGTSIVIPLQRGIPSRIMKAF |
| | vulneris | | AADFHAYAESLYNARGGTDEGGWTPTNSVATSNHLGGT |
| | | | AMDLNWSDHPMGKALDGYTAAETAVVRELLAFYEGMIFW |
| | | | GNDWNTPKDSMHFQMGYNTYTNQAKCNDFIKRKIRVDG |
| | | | FSTFRRGGTGGGTTPAPTPTENIYAQLGDNNDRVSSLQR |
| | | | FMNNNFASYSALDVDGDFGPATEEVIKNFQGRVGVAADG |
| | | | IVGPVTLAKLVEHGYVPLGTISTPPPAAAFAYPAHEEMVKQ |
| | | | LWEQAFGPQAKGWPDLFGKLADGSRGKYPVEAIADLHAD |
| | | | AGL |
| OCB59153 | Mycobacterium | 132 | MTENGWPACGPELLDRSAVPGTSIVIPLQRGIPSRIMKAF |
| (6X-His) | vulneris | | AADFHAYAESLYNARGGTDEGGWTPTNSVATSNHLGGT |
| | | | AMDLNWSDHPMGKALDGYTAAETAVVRELLAFYEGMIFW |
| | | | GNDWNTPKDSMHFQMGYNTYTNQAKCNDFIKRKIRVDG |
| | | | FSTFRRGGTGGGTTPAPTPTENIYAQLGDNNDRVSSLQR |
| | | | FMNNNFASYSALDVDGDFGPATEEVIKNFQGRVGVAADG |
| | | | IVGPVTLAKLVEHGYVPLGTISTPPPAAAFAYPAHEEMVKQ |
| | | | LWEQAFGPQAKGWPDLFGKLADGSRGKYPVEAIADLHAD |
| | | | AGLHHHHHH |
| OHU75990 | Mycobacteroides | 133 | MITENGWPSCSIAECDTNPIPGTDVRIPLQRGIPNIILKAFA |
| | chelonae | | ANLNSEIESVYNARGGTDEGGWTPTNSVATSNHLSGTAF |
| | | | DYNWTDHPMGPEADDPAAGWKGSSLIRGDQVPAIRELLR |
| | | | FFTYKGVQLVFWGNDWSTPKDSMHFQMGYGTYANQDL |
| | | | CREFIAKFIRADGFSTYRRGSSGGSWNAQVLAEATGLTIA |
| | | | RAAEILPQVAEGLRLSECVSPRRIAMWLAQIGHESDNFNA |
| | | | TEEYEKGDGGVTERWKYLGRTWIQITWLENYQGFSRWA |
| | | | YQKGIIPTPTYFVDRPKELAELQYAGIGPAWYWTVARANIN |
| | | | ALCDRGDLNGVTYLINGGYNGLSERQTRYNRAIALGDRLL |
| | | | ELLQEGDDMAQVPQDQLDRVFQEQTQEHESLSGYRDPD |
| | | | EGNIGTWCRIDRNKDLMIHELFTEWKAVQAGDLDSIRRLV |
| | | | RSAAGLGANTTPAFIANAKRMLKKVPAEYLQEGLAYLEST |
| | | | YPELLQAFISQNGAS |
| OHU75990 | Mycobacteroides | 134 | MITENGWPSCSIAECDTNPIPGTDVRIPLQRGIPNIILKAFA |
| (6X-His) | chelonae | | ANLNSEIESVYNARGGTDEGGWTPTNSVATSNHLSGTAF |
| | | | DYNWTDHPMGPEADDPAAGWKGSSLIRGDQVPAIRELLR |
| | | | FFTYKGVQLVFWGNDWSTPKDSMHFQMGYGTYANQDL |
| | | | CREFIAKFIRADGFSTYRRGSSGGSWNAQVLAEATGLTIA |
| | | | RAAEILPQVAEGLRLSECVSPRRIAMWLAQIGHESDNFNA |
| | | | TEEYEKGDGGVTERWKYLGRTWIQITWLENYQGFSRWA |
| | | | YQKGIIPTPTYFVDRPKELAELQYAGIGPAWYWTVARANIN |
| | | | ALCDRGDLNGVTYLINGGYNGLSERQTRYNRAIALGDRLL |
| | | | ELLQEGDDMAQVPQDQLDRVFQEQTQEHESLSGYRDPD |
| | | | EGNIGTWCRIDRNKDLMIHELFTEWKAVQAGDLDSIRRLV |
| | | | RSAAGLGANTTPAFIANAKRMLKKVPAEYLQEGLAYLEST |
| | | | YPELLQAFISQNGASHHHHHH |
| OYN80410 | Mycolicibacterium | 135 | MPVRYWPLGAGRIITSPFGPRDGGFHAGTDFGRAGGSA |
| | sphagni | | GMTVYAVQAGTVIYAGAAQGYGGPDPAGWLVIDSNDAE |
| | | | GGGCLEYGHIVRRPEIRVGTHVEAGQPIAQINPNSATNGG |
| | | | VAPHLHLSDMPGAYVPNAKQDPMPRLAGALEPESNSTPT |
| | | | QTEVPMSDPTWLPDVLRAAGLQCDIYPGAFDRGHGDFG |
| | | | EIWGVVCHHTGSFGETPRGIAEHPTLGLASQLYLSPDGKF |
| | | | TLCGVGIAWHAGAGSYPGLPDNNANFRTIGIEGANDGGG |
| | | | TPGKPHHQPWSNVQYDAYVAGVGAILKHLGQPASHSIDH |
| | | | KEWAGAAQGKWDRGGIDPNLFRNDVTAWSGGTLPPPPA |
| | | | TPVLVPGVPVEYANFGVIRRGDKGIRVVSLQTRLKRNYSK |
| | | | LVVDGDFGPDTEAKVRDYQSLHPPLVVDGQVGPATAAML |
| | | | KLIG |
| OYN80410 | Mycolicibacterium | 136 | MPVRYWPLGAGRIITSPFGPRDGGFHAGTDFGRAGGSA |
| (6X-His) | sphagni | | GMTVYAVQAGTVIYAGAAQGYGGPDPAGWLVIDSNDAE |
| | | | GGGCLEYGHIVRRPEIRVGTHVEAGQPIAQINPNSATNGG |
| | | | VAPHLHLSDMPGAYVPNAKQDPMPRLAGALEPESNSTPT |
| | | | QTEVPMSDPTWLPDVLRAAGLQCDIYPGAFDRGHGDFG |
| | | | EIWGVVCHHTGSFGETPRGIAEHPTLGLASQLYLSPDGKF |
| | | | TLCGVGIAWHAGAGSYPGLPDNNANFRTIGIEGANDGGG |
| | | | TPGKPHHQPWSNVQYDAYVAGVGAILKHLGQPASHSIDH |
| | | | KEWAGAAQGKWDRGGIDPNLFRNDVTAWSGGTLPPPPA |
| | | | TPVLVPGVPVEYANFGVIRRGDKGIRVVSLQTRLKRNYSK |
| | | | LVVDGDFGPDTEAKVRDYQSLHPPLVVDGQVGPATAAML |
| | | | KLIGHHHHHH |
| QRY48171 | Mycolicibacterium | 137 | MDVATLRRAMSPTSVSDSVLAQYLPHYVEAMKAAEITTVR |
| | boenickei | | RAAAFASQVGHESAGLRYMAEIATDGPDWSWDRTRYRG |
| | | | RGPIQLTWQGNYRKFGQWCKAKGYVTDSELFVNQPELV |
| | | | EQPKWGFLAASWYWLNGGPRPGQINGFADQGDILAVSR |
| | | | CVNGWVDNRLPNGWNDDDNSRLPRWNRCLAIGDALLTD |
| | | | QPAEETPVTWTGDPVWLEDVLHPALGDRLEALGGWRDR |
| | | | GHGDFKDIRGVMVHHTGNRNATAESIRDGRPDLPGPLSNI |
| | | | HIATDGTVTIVAVGVCWHAGIGSYPWLPTNNANWHMIGIE |
| | | | CAHDGNEQWPDAQIIALRDTCAALTVKLEQPASHVIGHKE |
| | | | YAGAAQGKWDPGNMDMNWFRGEVQKDMDGFVFPGEH |
| | | | PPIDPAPGPIVPPDYGKEVWDQLRIEWPQLGGRTLVDTVA |
| | | | AIGAKLGIEGCYDTKAGA |
| QRY48171 | Mycolicibacterium | 138 | MDVATLRRAMSPTSVSDSVLAQYLPHYVEAMKAAEITTVR |
| (6X-His) | boenickei | | RAAAFASQVGHESAGLRYMAEIATDGPDWSWDRTRYRG |
| | | | RGPIQLTWQGNYRKFGQWCKAKGYVTDSELFVNQPELV |
| | | | EQPKWGFLAASWYWLNGGPRPGQINGFADQGDILAVSR |
| | | | CVNGWVDNRLPNGWNDDDNSRLPRWNRCLAIGDALLTD |
| | | | QPAEETPVTWTGDPVWLEDVLHPALGDRLEALGGWRDR |
| | | | GHGDFKDIRGVMVHHTGNRNATAESIRDGRPDLPGPLSNI |
| | | | HIATDGTVTIVAVGVCWHAGIGSYPWLPTNNANWHMIGIE |
| | | | CAHDGNEQWPDAQIIALRDTCAALTVKLEQPASHVIGHKE |
| | | | YAGAAQGKWDPGNMDMNWFRGEVQKDMDGFVFPGEH |
| | | | PPIDPAPGPIVPPDYGKEVWDQLRIEWPQLGGRTLVDTVA |
| | | | AIGAKLGIEGCYDTKAGAHHHHHH |
| SIB68428 | Mycobacteroides | 139 | MPTVCAVPRFLPLKTGSFRWGSGFGPRWGTVHLGQDFE |
| | abscessus subsp. | | AADGTPIYAAQGGEVAYIGAATGFGQWIVIDHPAAEGAGT |
| | abscessus | | TVYGHMWNAYATGLKKGDRVSAGQLIGYVGANGQSTGP |
| | | | HLHFEVHPTAWRAGSQIDPRPWLAGALNPGGNVGVSGD |
| | | | PIWLEDVLRPALGDRLRTLPGWKTDGVGGTMGTIWGVV |
| | | | WHHTGNAAEKPESISKGRPDLPGPLAQIHIAPDGIVTIVAV |
| | | | GPCNHAGRGSWPGLPTDNANAYTIGIECAYPRDTSLTEE |
| | | | TITREPWPAAQIISMRDVGAALTKHLQVPVSHNLSHAEWA |
| | | | RFGPAGVRQYKWDPGNLDMNWFRGEIQKDIDGYQFPGE |
| | | | TPTTTLPSSPDYPREIWDQLRIAWPQLGARTLVDAVAVIG |
| | | | KQLGIADFAPIGKDAS |
| SIB68428 | Mycobacteroides | 140 | MPTVCAVPRFLPLKTGSFRWGSGFGPRWGTVHLGQDFE |
| (6X-His) | abscessus subsp. | | AADGTPIYAAQGGEVAYIGAATGFGQWIVIDHPAAEGAGT |
| | abscessus | | TVYGHMWNAYATGLKKGDRVSAGQLIGYVGANGQSTGP |
| | | | HLHFEVHPTAWRAGSQIDPRPWLAGALNPGGNVGVSGD |
| | | | PIWLEDVLRPALGDRLRTLPGWKTDGVGGTMGTIWGVV |
| | | | WHHTGNAAEKPESISKGRPDLPGPLAQIHIAPDGIVTIVAV |
| | | | GPCNHAGRGSWPGLPTDNANAYTIGIECAYPRDTSLTEE |
| | | | TITREPWPAAQIISMRDVGAALTKHLQVPVSHNLSHAEWA |
| | | | RFGPAGVRQYKWDPGNLDMNWFRGEIQKDIDGYQFPGE |
| | | | TPTTTLPSSPDYPREIWDQLRIAWPQLGARTLVDAVAVIG |
| | | | KQLGIADFAPIGKDASHHHHHH |
| UJL31707 | Mycolicibacterium | 141 | MHRGIDFEAADGTPIYAAGSGIVAHIGPAQGFGQWIVIRHD |
| | vanbaalenii | | SGESTVYGHMWDAHALGLRQGDRVFAGQHIAFVGSNGQ |
| | | | SSGPHLHFEVHPGAWRAGSQIDPRPWLNGAMDPRTRGG |
| | | | TVSGWTGDPVWLADVLKADGEIDVRELGGWRDRGHGDF |
| | | | KDIRGIMVHHTGGPASAESIRDGRPDLRGPLSQLHISRDG |
| | | | VVSVVAAGVSWHAGTGSLPWIPANMGNWHLIGIECEWPY |
| | | | RGGIGEHNQRDEPWRRPQILAIRNTCAAILRKLAFGADRIT |
| | | | THKEYAGRSQGKWDPGNMDPNWLRDEIAKDLRGVKFPG |
| | | | EGTPDYTPPPTPVPVPPVDQYAGVLLYRGMRGPAVVKLQ |
| | | | QRLKDTIVSKLVVDGDFGPKTEAAVRAFQSSKWRRPPLV |
| | | | ADGVVGPATAAQLGLVL |
| UJL31707 | Mycolicibacterium | 142 | MHRGIDFEAADGTPIYAAGSGIVAHIGPAQGFGQWIVIRHD |
| (6X-His) | vanbaalenii | | SGESTVYGHMWDAHALGLRQGDRVFAGQHIAFVGSNGQ |
| | | | SSGPHLHFEVHPGAWRAGSQIDPRPWLNGAMDPRTRGG |
| | | | TVSGWTGDPVWLADVLKADGEIDVRELGGWRDRGHGDF |
| | | | KDIRGIMVHHTGGPASAESIRDGRPDLRGPLSQLHISRDG |
| | | | VVSVVAAGVSWHAGTGSLPWIPANMGNWHLIGIECEWPY |
| | | | RGGIGEHNQRDEPWRRPQILAIRNTCAAILRKLAFGADRIT |
| | | | THKEYAGRSQGKWDPGNMDPNWLRDEIAKDLRGVKFPG |
| | | | EGTPDYTPPPTPVPVPPVDQYAGVLLYRGMRGPAVVKLQ |
| | | | QRLKDTIVSKLVVDGDFGPKTEAAVRAFQSSKWRRPPLV |
| | | | ADGVVGPATAAQLGLVLHHHHHH |
| VTP00005 | Mycobacterium | 143 | MTMVGGFDVHRQQITFDYVDDDGLVRWGQIRPATLSTLR |
| | riyadhense | | RWLSERCPDGDAEFALEGCTGWRYVSEELAAAGVRVHV |
| | | | GDPAEIAALRGRKKRAKTDRADARLLRTLLLQGRFPESWI |
| | | | PPAHVLEIGNLGRLYCTLMDERRAWQQRIHAQLFHQGCP |
| | | | PVKALLSARGREALASAPLSPAGRQYVDAALRRIDELTPEI |
| | | | DALRTQLVDFARRQAGCRALQRHYGIGWLCAAIMWAEIG |
| | | | DARRFTSSDQLVRDAGLDVTVHSSDAKRSPGHLSRQGSP |
| | | | DSAATHPRQPARRAPHQSSCHRTSSVDPDKAGRRHARL |
| | | | SSRAPHSRTGGSRWLELQRLSLVPPDREYAADPPRPQG |
| | | | RSSSLRCGRSTLTPTPLHRPMPGMGNEAKPQNNDQRIPT |
| | | | FTGVPDTDKDSSMPQTGTWTGDPVWLVDVLRAEGVNPIE |
| | | | YPGWRDRGHGDFKDIRGVMVHHTGSDSASAASIAQGRP |
| | | | DLAGPLSQLHIARDGTVTVVAVGVAWHAGVGMYPWLPTN |
| | | | MGNWHLIGIECANTGTSPTAPHRQNWPDAQYFAMVNCC |
| | | | AAINRRLAQTSARTIGHKEYAGRAQGKWDPGAIDMDILRR |
| | | | DIQDQIGRVAKPAPTPRPPLPVGEYADILLFRGSEGPQVA |
| | | | QLQRRLNEQGAGLVVDGIFGPNTEAAVREFQRRTRGLKV |
| | | | DGIVGPATAAAPRLKVGPGPE |
| VTP00005 | Mycobacterium | 144 | MTMVGGFDVHRQQITFDYVDDDGLVRWGQIRPATLSTLR |
| (6X-His) | riyadhense | | RWLSERCPDGDAEFALEGCTGWRYVSEELAAAGVRVHV |
| | | | GDPAEIAALRGRKKRAKTDRADARLLRTLLLQGRFPESWI |
| | | | PPAHVLEIGNLGRLYCTLMDERRAWQQRIHAQLFHQGCP |
| | | | PVKALLSARGREALASAPLSPAGRQYVDAALRRIDELTPEI |
| | | | DALRTQLVDFARRQAGCRALQRHYGIGWLCAAIMWAEIG |
| | | | DARRFTSSDQLVRDAGLDVTVHSSDAKRSPGHLSRQGSP |
| | | | DSAATHPRQPARRAPHQSSCHRTSSVDPDKAGRRHARL |
| | | | SSRAPHSRTGGSRWLELQRLSLVPPDREYAADPPRPQG |
| | | | RSSSLRCGRSTLTPTPLHRPMPGMGNEAKPQNNDQRIPT |
| | | | FTGVPDTDKDSSMPQTGTWTGDPVWLVDVLRAEGVNPIE |
| | | | YPGWRDRGHGDFKDIRGVMVHHTGSDSASAASIAQGRP |
| | | | DLAGPLSQLHIARDGTVTVVAVGVAWHAGVGMYPWLPTN |
| | | | MGNWHLIGIECANTGTSPTAPHRQNWPDAQYFAMVNCC |
| | | | AAINRRLAQTSARTIGHKEYAGRAQGKWDPGAIDMDILRR |
| | | | DIQDQIGRVAKPAPTPRPPLPVGEYADILLFRGSEGPQVA |
| | | | QLQRRLNEQGAGLVVDGIFGPNTEAAVREFQRRTRGLKV |
| | | | DGIVGPATAAAPRLKVGPGPEHHHHHH |
| WP_064896079 | Mycolicibacterium | 145 | MSFINKQGYWISENGWRMCDTAELDYTAVPGTSFNLGVR |
| | conceptionense | | KGAPNIILKAAIARLHREVEPMITSQIGCYTGTNSMPNSNH |
| | | | NSATAFDYNWNKHPYQEWGTWGANRAKVDRIVADFRGI |
| | | | LEFGGNWTSPRDEMHFELHFAEGHAGTEQLATDLRNGL |
| | | | WGIWAPGQAPEPGAPSTPIPDGYLQIGSEGDQVRKLQVG |
| | | | MNKVFPNYRAMPLDEDGIFGPMTEAAVTEFQQRSLIGAD |
| | | | GIVGPETKARLATYGIVLDGASAPTSPPAVPPAFVYPSTDE |
| | | | MTKQLWEQAFGPQAKGWPDLFGKLADGSRGKYTVEAIA |
| | | | DLHADAGL |
| WP_064896079 | Mycolicibacterium | 146 | MSFINKQGYWISENGWRMCDTAELDYTAVPGTSFNLGVR |
| (6X-His) | conceptionense | | KGAPNIILKAAIARLHREVEPMITSQIGCYTGTNSMPNSNH |
| | | | NSATAFDYNWNKHPYQEWGTWGANRAKVDRIVADFRGI |
| | | | LEFGGNWTSPRDEMHFELHFAEGHAGTEQLATDLRNGL |
| | | | WGIWAPGQAPEPGAPSTPIPDGYLQIGSEGDQVRKLQVG |
| | | | MNKVFPNYRAMPLDEDGIFGPMTEAAVTEFQQRSLIGAD |
| | | | GIVGPETKARLATYGIVLDGASAPTSPPAVPPAFVYPSTDE |
| | | | MTKQLWEQAFGPQAKGWPDLFGKLADGSRGKYTVEAIA |
| | | | DLHADAGLHHHHHH |
| WP_078323605 | Mycobacteroides | 147 | MSFRNVYGNDWSENGWRMCNRDECVLVDGPFMNTAPL |
| | salmoniphilum | | RRGPAEIILGDFVRRYHAQVAPVISPVWGWSLYNDVDDS |
| | | | NHLSATGVDINAPQWPWGEKRMPQWLIDRVNALLADYYI |
| | | | DGECGIYWGRNWNRPDEMHFQLNWPEGDPRYARLIAKI |
| | | | QGKPPVVVNPPNPGGSVGADGKAILRIGSKGAEVRQLQA |
| | | | GMNVVFEDYSHLDEDGEYGPMTAAVVAEFQRRSHLKITD |
| | | | YGVVGAITRKELNRYNILGAAKGTAPSVPAPPEPEGEAPR |
| | | | PIYIYSAAGTGAQWWQGPPFEAGQWCKGLNIHHQPVGY |
| | | | PAGGFLGLMGGDPKISYNDSITNLGDELERLIDSNPDIDNP |
| | | | DIEFWFFGYSQSADGMKRKVTELFGDGGKYSHLRSRING |
| | | | LILFGDPTRAPGPTKVGNNPPGWGIARWDAPEWVDKLTW |
| | | | SITTHNDLYACTTDDTLVKYFYPWFIRAETELPFVIYSAKIII |
| | | | PALLNLAAPFLGGALGGVATPILSAITGLAGSFIGQLLGGIT |
| | | | GGDDPDPELIKLLSIQGILSNIPQLIKTLVALQGIQTHGEYHL |
| | | | PKPEFDNRTGIQVACDIVRDFRR |
| WP_078323605 | Mycobacteroides | 148 | MSFRNVYGNDWSENGWRMCNRDECVLVDGPFMNTAPL |
| (6X-His) | salmoniphilum | | RRGPAEIILGDFVRRYHAQVAPVISPVWGWSLYNDVDDS |
| | | | NHLSATGVDINAPQWPWGEKRMPQWLIDRVNALLADYYI |
| | | | DGECGIYWGRNWNRPDEMHFQLNWPEGDPRYARLIAKI |
| | | | QGKPPVVVNPPNPGGSVGADGKAILRIGSKGAEVRQLQA |
| | | | GMNVVFEDYSHLDEDGEYGPMTAAVVAEFQRRSHLKITD |
| | | | YGVVGAITRKELNRYNILGAAKGTAPSVPAPPEPEGEAPR |
| | | | PIYIYSAAGTGAQWWQGPPFEAGQWCKGLNIHHQPVGY |
| | | | PAGGFLGLMGGDPKISYNDSITNLGDELERLIDSNPDIDNP |
| | | | DIEFWFFGYSQSADGMKRKVTELFGDGGKYSHLRSRING |
| | | | LILFGDPTRAPGPTKVGNNPPGWGIARWDAPEWVDKLTW |
| | | | SITTHNDLYACTTDDTLVKYFYPWFIRAETELPFVIYSAKIII |
| | | | PALLNLAAPFLGGALGGVATPILSAITGLAGSFIGQLLGGIT |
| | | | GGDDPDPELIKLLSIQGILSNIPQLIKTLVALQGIQTHGEYHL |
| | | | PKPEFDNRTGIQVACDIVRDFRRHHHHHH |
| WP_079616971 | Mycobacteroides | 149 | MSFVWFRPEGPLRTREQIAREVHAVSLARGLDELASVLTL |
| | abscessus | | MCIDVEAGADDNNGERQWWCPWNANDPTSKNYPHDSQ |
| | | | SDDGRSVGYLQQQNGAAGEVVSGSDNWWGPMRSRMTL |
| | | | ALAVDVFQTRLADDYGRAAGNPKLAGEFVQRVQRSGYP |
| | | | DRYAQRWDEAWAVLRRALAQGPVTPKPPLPPIGTGSPIT |
| | | | RSRLTSNRYVGRGGKTPRWIVVHTQEGGRSAWDLAGFL |
| | | | KSTEGQGVRSVSYNACVDDTETVLTVNWDDTPWSAVNA |
| | | | NPYAFHICMAGSYSGWDRGKWLETDARDGKNEDLQLTR |
| | | | TAQLIAWLCRTYDIPADYIGGSGIPWGRDGICGHRDFGSW |
| | | | GGGHTDPGPDFPWDELIRRVRLYLDTNTGDQDMAQVPQ |
| | | | SEWQEVIDYVRAQNTPIPSASPLRHLGEGNVNTRANLARA |
| | | | IDANQHVTAVVTLAKEGHTPSIALLWEVSTAADAPGKYPD |
| | | | RQEDAKLAKTLLASISKTKKAVAAEDIEAWLDAEKAAA |
| WP_079616971 | Mycobacteroides | 150 | MSFVWFRPEGPLRTREQIAREVHAVSLARGLDELASVLTL |
| (6X-His) | abscessus | | MCIDVEAGADDNNGERQWWCPWNANDPTSKNYPHDSQ |
| | | | SDDGRSVGYLQQQNGAAGEVVSGSDNWWGPMRSRMTL |
| | | | ALAVDVFQTRLADDYGRAAGNPKLAGEFVQRVQRSGYP |
| | | | DRYAQRWDEAWAVLRRALAQGPVTPKPPLPPIGTGSPIT |
| | | | RSRLTSNRYVGRGGKTPRWIVVHTQEGGRSAWDLAGFL |
| | | | KSTEGQGVRSVSYNACVDDTETVLTVNWDDTPWSAVNA |
| | | | NPYAFHICMAGSYSGWDRGKWLETDARDGKNEDLQLTR |
| | | | TAQLIAWLCRTYDIPADYIGGSGIPWGRDGICGHRDFGSW |
| | | | GGGHTDPGPDFPWDELIRRVRLYLDTNTGDQDMAQVPQ |
| | | | SEWQEVIDYVRAQNTPIPSASPLRHLGEGNVNTRANLARA |
| | | | IDANQHVTAVVTLAKEGHTPSIALLWEVSTAADAPGKYPD |
| | | | RQEDAKLAKTLLASISKTKKAVAAEDIEAWLDAEKAAAHH |
| | | | HHHH |
| WP_079634229 | Mycobacteroides | 151 | MSFVWLADKPLRSREEIAQIVHAVSLARGLDELASAMVLM |
| | abscessus | | CIDVEVGANDRNGKRQWWCPWNAKDPSSQKFPHDSES |
| | | | NDGRSVGYFQQQNGRAGEVLPVDDRDNWWGSMTSRM |
| | | | TLALAADTFLDRLSDDYRGAADNPALAGQFVQRVQGSAF |
| | | | PDRYAQSWNEAWSVLRRALDGTTEPSEPGVLTPTPGFS |
| | | | GDPFWLADVLRAEGLNVVEMDGWRERGEGDQGVLWGA |
| | | | VFHHTGNVNETPEGIAFHPDLGLAAHLLIRPDGTVWVCGI |
| | | | GMANHAGVGSWPGIRTDNANPVTIGVEVAILPEKDAPHRT |
| | | | GWPAVQYDATVKTFAAILRKLALGSDRAISHKEWAQLGPA |
| | | | GYRQGKWDPGAIDMNIFRADVQAQIGSNTGGFLMALSDT |
| | | | EQREILSFVREMKTQVASLSPFRHLGEEAANNIPGFVRVID |
| | | | ANSHVEIVEKLAGYGDQGALALLHEIADADLERYPDRRND |
| | | | AELARRVLAKVAK |
| WP_079634229 | Mycobacteroides | 152 | MSFVWLADKPLRSREEIAQIVHAVSLARGLDELASAMVLM |
| (6X-His) | abscessus | | CIDVEVGANDRNGKRQWWCPWNAKDPSSQKFPHDSES |
| | | | NDGRSVGYFQQQNGRAGEVLPVDDRDNWWGSMTSRM |
| | | | TLALAADTFLDRLSDDYRGAADNPALAGQFVQRVQGSAF |
| | | | PDRYAQSWNEAWSVLRRALDGTTEPSEPGVLTPTPGFS |
| | | | GDPFWLADVLRAEGLNVVEMDGWRERGEGDQGVLWGA |
| | | | VFHHTGNVNETPEGIAFHPDLGLAAHLLIRPDGTVWVCGI |
| | | | GMANHAGVGSWPGIRTDNANPVTIGVEVAILPEKDAPHRT |
| | | | GWPAVQYDATVKTFAAILRKLALGSDRAISHKEWAQLGPA |
| | | | GYRQGKWDPGAIDMNIFRADVQAQIGSNTGGFLMALSDT |
| | | | EQREILSFVREMKTQVASLSPFRHLGEEAANNIPGFVRVID |
| | | | ANSHVEIVEKLAGYGDQGALALLHEIADADLERYPDRRND |
| | | | AELARRVLAKVAKHHHHHH |
| WP_083169868 | Mycobacterium | 153 | MALGMILENGWPECDLVDCDYATIPGTPLRLPFQKGHPFII |
| | aquaticum | | LQAFLRDLDQYIEPVMNARGITDEGSWTEDNSVYTSNHK |
| | | | GATAFDYNWDDHPMGRAGAGWDGSVLIAGDQVPAVQEL |
| | | | LAWYEGMVFWGNNWSSPKDSMHFQMGYDTYGPANAAR |
| | | | VQNFIDRKIRADGYSTWRRGGTARGGGVVPPVAVPVQTG |
| | | | LTANLLQSIGGYRKDMTLARYQALLPELIDAFHFADLNTID |
| | | | RRAMGIAQLFHESGALRYQEEIADGSAYEGRTDLGNTQR |
| | | | GDGKRYKGRDFLQITGRSNYTALSAWAFARKIPGADSPTF |
| | | | FVDRPELLATDRFAFLGFAWYWTTRRNKAGQSLNDMAD |
| | | | ARNIDGATLMVNGGYNGLDSRKTFYARALAANADLLDPE |
| | | | PVDPLEELLMSDRKVPSASIYATPGEEDIPLVELLRAIDAAL |
| | | | HRTAIVEPDAELGDPDAIDRMLRTAAGKGQYGTLPGPVN |
| | | | HAKAKLAKIAAANPPALLYVARAAKAGDVAALGVITDLQNT |
| | | | NPAVLQAFVAAQKGAN |
| WP_083169868 | Mycobacterium | 154 | MALGMILENGWPECDLVDCDYATIPGTPLRLPFQKGHPFII |
| (6X-His) | aquaticum | | LQAFLRDLDQYIEPVMNARGITDEGSWTEDNSVYTSNHK |
| | | | GATAFDYNWDDHPMGRAGAGWDGSVLIAGDQVPAVQEL |
| | | | LAWYEGMVFWGNNWSSPKDSMHFQMGYDTYGPANAAR |
| | | | VQNFIDRKIRADGYSTWRRGGTARGGGVVPPVAVPVQTG |
| | | | LTANLLQSIGGYRKDMTLARYQALLPELIDAFHFADLNTID |
| | | | RRAMGIAQLFHESGALRYQEEIADGSAYEGRTDLGNTQR |
| | | | GDGKRYKGRDFLQITGRSNYTALSAWAFARKIPGADSPTF |
| | | | FVDRPELLATDRFAFLGFAWYWTTRRNKAGQSLNDMAD |
| | | | ARNIDGATLMVNGGYNGLDSRKTFYARALAANADLLDPE |
| | | | PVDPLEELLMSDRKVPSASIYATPGEEDIPLVELLRAIDAAL |
| | | | HRTAIVEPDAELGDPDAIDRMLRTAAGKGQYGTLPGPVN |
| | | | HAKAKLAKIAAANPPALLYVARAAKAGDVAALGVITDLQNT |
| | | | NPAVLQAFVAAQKGANHHHHHH |
| WP_085143062 | Mycolicibacterium | 155 | MVLRTENGWYQVPASSCEWTTIPGTNVTLQIQKGWPLTI |
| | conceptionense | | MRAFAADFNAYVEPLRDPDSACWTPTNSVATSNHLSGTA |
| | | | MDLNWNSHPFQVLNAGFDAAKIATIRELLAFYEDTIFWGN |
| | | | DWTTPKDAMHFQMGYDTYNNSHTQDFINRKIRPDGYSTF |
| | | | RRGEAPSVDVAQVLSDAMGGRLSLDRYAQLLPAVTASLL |
| | | | ACDCTNVNRIAMWCAQIGHESAGLYYTEEIASGAAYEGR |
| | | | ADLGNTQPGDGVRFKGRSWIQITGRSNYTRLSAWAFSKG |
| | | | LIPSSTYFVDNPTALASDEYAGLGAAWYWVVARPDINALS |
| | | | DARDLVTVTKRINGGTNGLSDRQDRYNRALAMGDQLLAL |
| | | | TTGGDDMFTDADRDLLKQIAEYRRKSLSPLRWPYEGAVN |
| | | | TCAGFAWTADANIHMVLVEKLAVEYGDPQSVALLLAVSRT |
| | | | DEPGREDDTALAAKILTKVDPDAVSAGERWLAEHRVD |
| WP_085143062 | Mycolicibacterium | 156 | MVLRTENGWYQVPASSCEWTTIPGTNVTLQIQKGWPLTI |
| (6X-His) | conceptionense | | MRAFAADFNAYVEPLRDPDSACWTPTNSVATSNHLSGTA |
| | | | MDLNWNSHPFQVLNAGFDAAKIATIRELLAFYEDTIFWGN |
| | | | DWTTPKDAMHFQMGYDTYNNSHTQDFINRKIRPDGYSTF |
| | | | RRGEAPSVDVAQVLSDAMGGRLSLDRYAQLLPAVTASLL |
| | | | ACDCTNVNRIAMWCAQIGHESAGLYYTEEIASGAAYEGR |
| | | | ADLGNTQPGDGVRFKGRSWIQITGRSNYTRLSAWAFSKG |
| | | | LIPSSTYFVDNPTALASDEYAGLGAAWYWVVARPDINALS |
| | | | DARDLVTVTKRINGGTNGLSDRQDRYNRALAMGDQLLAL |
| | | | TTGGDDMFTDADRDLLKQIAEYRRKSLSPLRWPYEGAVN |
| | | | TCAGFAWTADANIHMVLVEKLAVEYGDPQSVALLLAVSRT |
| | | | DEPGREDDTALAAKILTKVDPDAVSAGERWLAEHRVDHH |
| | | | HHHH |
| WP_135361703 | Mycolicibacterium | 157 | MTEKVLPYDRAVVPQEYGWSCGPAATQVVLNSRGIVVSE |
| | peregrinum | | TSLLAQIEAVENPGRGDDRDGTDYVGLIERVLDNIVPDAS |
| | | | YTSVYLERDPATAAQKEALWTNLKRSIDGGFGVVMNWVA |
| | | | PPSNKPRGVKGSVSPRYSGGTTYHYVAAMGYDDNPAAR |
| | | | AVWIADSGFQPQGYWISFDQCATLIPPKGYAFADVTPATG |
| | | | GPSTPTTPTPSADQVSVLAQVMSPSVVARDRFAALLPAV |
| | | | AQCLAECGCTTVERIAMWAAQIGTESGGLRYMEEIASGA |
| | | | EYEGRCSDLGNCQPGDGIRFKGRGPIQVTGRGHYTNLSQ |
| | | | WAHGKGLVPTPTFFVDDPGQLASDRYGFIGVTWYWTTQ |
| | | | RPMNDYADRRDIEGGSRAVNGTNPKTGRANGIDDRIGRY |
| | | | NRALAMGDALLTLTTTTGSGDITMSAAEELEAQSRGVFPP |
| | | | SDEQKRGGWPQPWRFVYSRFARPFNSIVKDAARGPWG |
| | | | RVQPDNTWQNGHTDSDEQTVTIGEQIAWRNEFSDGITRD |
| | | | HGDVMIELMEDLIARRKSAGQSTSPATDALKAAQKS |
| WP_135361703 | Mycolicibacterium | 158 | MTEKVLPYDRAVVPQEYGWSCGPAATQVVLNSRGIVVSE |
| (6X-His) | peregrinum | | TSLLAQIEAVENPGRGDDRDGTDYVGLIERVLDNIVPDAS |
| | | | YTSVYLERDPATAAQKEALWTNLKRSIDGGFGVVMNWVA |
| | | | PPSNKPRGVKGSVSPRYSGGTTYHYVAAMGYDDNPAAR |
| | | | AVWIADSGFQPQGYWISFDQCATLIPPKGYAFADVTPATG |
| | | | GPSTPTTPTPSADQVSVLAQVMSPSVVARDRFAALLPAV |
| | | | AQCLAECGCTTVERIAMWAAQIGTESGGLRYMEEIASGA |
| | | | EYEGRCSDLGNCQPGDGIRFKGRGPIQVTGRGHYTNLSQ |
| | | | WAHGKGLVPTPTFFVDDPGQLASDRYGFIGVTWYWTTQ |
| | | | RPMNDYADRRDIEGGSRAVNGTNPKTGRANGIDDRIGRY |
| | | | NRALAMGDALLTLTTTTGSGDITMSAAEELEAQSRGVFPP |
| | | | SDEQKRGGWPQPWRFVYSRFARPFNSIVKDAARGPWG |
| | | | RVQPDNTWQNGHTDSDEQTVTIGEQIAWRNEFSDGITRD |
| | | | HGDVMIELMEDLIARRKSAGQSTSPATDALKAAQKSHHH |
| | | | HHH |
| WP_184445281 | Mycobacterium | 159 | MSFTQFLQDDPLLAREQIMAIAIDVAKTLGMPDVKGAAILN |
| | sp. AZCC_0083 | | VMAVSVEVGVEDNDPPHARRFWCPANHNDGDSFNYSHD |
| | | | SVSNDNRSIGYLQQQKGPNGELWWGTTAQQMDPHQAFI |
| | | | SFQTRLKKRGYDASNAQSAGEAAQAIQGSAFPDRYREQ |
| | | | WDDINALYDAVAGTITAPTTPTQPSFSFTEKNIIDGENASN |
| | | | RQGHNPRLFVLHTEEGNLLGQALDNWMDNNEVSYHYAV |
| | | | DPDASGAVAWDLVDTDLSSWSVLSANPDSINLVFAGSYA |
| | | | AMSRTDWLSKYGKAIKVAAYLAVQDCRKYGIETTVRVGFA |
| | | | AGGYSSLKTSNGITDHYGITKGLGIGTHTDVGPNFPWDVF |
| | | | ASYVAQFANGTTEEDDMFTDADRALLTRVHFELTNKWNS |
| | | | RSIYAEPGEGPVDTLAGMVLNDDGMEHSELVERLAVLGD |
| | | | PDSLRRVIRVAAGEGVVTDAATVARAAKILAEVPADILTAY |
| | | | EAAAK |
| WP_184445281 | Mycobacterium | 160 | MSFTQFLQDDPLLAREQIMAIAIDVAKTLGMPDVKGAAILN |
| (6X-His) | sp. AZCC_0083 | | VMAVSVEVGVEDNDPPHARRFWCPANHNDGDSFNYSHD |
| | | | SVSNDNRSIGYLQQQKGPNGELWWGTTAQQMDPHQAFI |
| | | | SFQTRLKKRGYDASNAQSAGEAAQAIQGSAFPDRYREQ |
| | | | WDDINALYDAVAGTITAPTTPTQPSFSFTEKNIIDGENASN |
| | | | RQGHNPRLFVLHTEEGNLLGQALDNWMDNNEVSYHYAV |
| | | | DPDASGAVAWDLVDTDLSSWSVLSANPDSINLVFAGSYA |
| | | | AMSRTDWLSKYGKAIKVAAYLAVQDCRKYGIETTVRVGFA |
| | | | AGGYSSLKTSNGITDHYGITKGLGIGTHTDVGPNFPWDVF |
| | | | ASYVAQFANGTTEEDDMFTDADRALLTRVHFELTNKWNS |
| | | | RSIYAEPGEGPVDTLAGMVLNDDGMEHSELVERLAVLGD |
| | | | PDSLRRVIRVAAGEGVVTDAATVARAAKILAEVPADILTAY |
| | | | EAAAKHHHHHH |
| WP_200996520 | Mycobacteroides | 161 | MGRRNVYGYDYSSNGWPMVDEGSCTWVTVPGTSVSLQI |
| | chelonae | | QNGQPLAILRAFAADFNAYVEPLRDPDSASWTPGNSVPT |
| | | | SNHLSGTAMDLNWDSHPFQIANAGFSAAQIATVKEIQAFY |
| | | | EGTVFWGNDWSDPKDAMHFQLASLANGGDINTYQNAHT |
| | | | ADFIARKIRADGFSTFRRGNKPIGGVSILAAATGLPEARAA |
| | | | EILPAVSDGLKASQCTNVNRIAMWLAQVGHESGSFVYTEE |
| | | | IASGAAYEGRADLGNTQPGDGVRFKGRSWIQITGRNNYG |
| | | | LFSQWAFRQRLVQSPTYFVDHSTELADLKWAGIGAAWY |
| | | | WTVARTDINALSDRQDLETVTRRINGGTNGLADRRDRYN |
| | | | RALLQGDALLQLLAPAEPLDPIEELLMSGLAVPSMSIYADP |
| | | | DEPDVPVVDMIRALDAHGPHEPYVENQARLGDPDALRRV |
| | | | ARTAAGRGKYGTASGPVNQAKSVLEGIRVTNPAVLEQFD |
| | | | AQSGA |
| WP_200996520 | Mycobacteroides | 162 | MGRRNVYGYDYSSNGWPMVDEGSCTWVTVPGTSVSLQI |
| (6X-His) | chelonae | | QNGQPLAILRAFAADFNAYVEPLRDPDSASWTPGNSVPT |
| | | | SNHLSGTAMDLNWDSHPFQIANAGFSAAQIATVKEIQAFY |
| | | | EGTVFWGNDWSDPKDAMHFQLASLANGGDINTYQNAHT |
| | | | ADFIARKIRADGFSTFRRGNKPIGGVSILAAATGLPEARAA |
| | | | EILPAVSDGLKASQCTNVNRIAMWLAQVGHESGSFVYTEE |
| | | | IASGAAYEGRADLGNTQPGDGVRFKGRSWIQITGRNNYG |
| | | | LFSQWAFRQRLVQSPTYFVDHSTELADLKWAGIGAAWY |
| | | | WTVARTDINALSDRQDLETVTRRINGGTNGLADRRDRYN |
| | | | RALLQGDALLQLLAPAEPLDPIEELLMSGLAVPSMSIYADP |
| | | | DEPDVPVVDMIRALDAHGPHEPYVENQARLGDPDALRRV |
| | | | ARTAAGRGKYGTASGPVNQAKSVLEGIRVTNPAVLEQFD |
| | | | AQSGAHHHHHH |
| WP_211155892 | Mycolicibacterium | 163 | MANRIVYGLTHSSNGWPMVDEGSCTWVKIPGTSVTLQIQ |
| | septicum | | NGQPLAILRAFAADFNAYVEPLRDADSACWTPTNSVSSS |
| | | | NHLSGTAMDLNWGDHPFYSNYGGYTPAEIATVRELLDFY |
| | | | EGFMFWGQDWTNPKDCMHFQLSSLANGGPVNTYGNPA |
| | | | VDNFIARKIRADGFSTFRRGNAPVGGAPVLAAATGLSEAR |
| | | | AAEILPAVQSGLRQSDATTVPRIAQWLAQVGHESVSFYYT |
| | | | EEIAKNGRYAPYIGRTWIQITWDYNYRAFSEWCFDRGLVP |
| | | | TRDYFVVNYQRLADLEWAGLGASWYWTEQRPMNALVDA |
| | | | GENASWNGYRGFSAVTAAINGGTNGLADRQARYNRALAL |
| | | | GDRLLTLIDSDSGDDLASVPQDQWDRVFREQTQKLPSRS |
| | | | ALRRLGEGVIDTWAGIDLNQDGNLHVLLVKTLAELGDTDS |
| | | | LDLLREVASADPVRYPDRQKDRQLALRILADIEANKPGVL |
| | | | QNYLAEKGIA |
| WP_211155892 | Mycolicibacterium | 164 | MANRIVYGLTHSSNGWPMVDEGSCTWVKIPGTSVTLQIQ |
| (6X-His) | septicum | | NGQPLAILRAFAADFNAYVEPLRDADSACWTPTNSVSSS |
| | | | NHLSGTAMDLNWGDHPFYSNYGGYTPAEIATVRELLDFY |
| | | | EGFMFWGQDWTNPKDCMHFQLSSLANGGPVNTYGNPA |
| | | | VDNFIARKIRADGFSTFRRGNAPVGGAPVLAAATGLSEAR |
| | | | AAEILPAVQSGLRQSDATTVPRIAQWLAQVGHESVSFYYT |
| | | | EEIAKNGRYAPYIGRTWIQITWDYNYRAFSEWCFDRGLVP |
| | | | TRDYFVVNYQRLADLEWAGLGASWYWTEQRPMNALVDA |
| | | | GENASWNGYRGFSAVTAAINGGTNGLADRQARYNRALAL |
| | | | GDRLLTLIDSDSGDDLASVPQDQWDRVFREQTQKLPSRS |
| | | | ALRRLGEGVIDTWAGIDLNQDGNLHVLLVKTLAELGDTDS |
| | | | LDLLREVASADPVRYPDRQKDRQLALRILADIEANKPGVL |
| | | | QNYLAEKGIAHHHHHH |
| WP_211695648 | Mycobacterium | 165 | MKDDYARAILHAGRELGITPRGIVIAFATVYVESDWIMYAN |
| | spongiae | | HKVPESLNLPHERVGSDGRSVGLFQQQVVWGNGAWWW |
| | | | GDAATCMDPHKSARLFFQRLARLDYNSDANSPGWYAQQ |
| | | | VQRSAFPDRYDPRMSAAQALYNRILGTTSARPDESPEPL |
| | | | AGWQGDPVWLADVLREALGDRLVVEAGWQGRGNGAGP |
| | | | NRQMDDIWGVMIHHTGNDRETVAAIRDGVEQPGGFLAGP |
| | | | LAQCLIKPDGRCHLVAVGPCNHAGQGYYPGVGTNTGNIR |
| | | | LIGFECAWPTVNPDGSYNRGQPWPDAQIHTMRDAATAVV |
| | | | RRLGYGSDRVIGHKEYATRAPNVKWDPGNLSMDWFRLQ |
| | | | VAKDLRGEFDPKPADQPRVEVPEPSPTGIPPDFTQQMFL |
| | | | QMSGRWEMLGWQTIVESLAEIRDATIGSSDAGKPGFTLG |
| | | | GRPQTPAAVLAARKTPGTNASATKASARKAPAEKAPAKK |
| | | | ARARKVRAKKASAEKAAARG |
| WP_211695648 | Mycobacterium | 166 | MKDDYARAILHAGRELGITPRGIVIAFATVYVESDWIMYAN |
| (6X-His) | spongiae | | HKVPESLNLPHERVGSDGRSVGLFQQQVVWGNGAWWW |
| | | | GDAATCMDPHKSARLFFQRLARLDYNSDANSPGWYAQQ |
| | | | VQRSAFPDRYDPRMSAAQALYNRILGTTSARPDESPEPL |
| | | | AGWQGDPVWLADVLREALGDRLVVEAGWQGRGNGAGP |
| | | | NRQMDDIWGVMIHHTGNDRETVAAIRDGVEQPGGFLAGP |
| | | | LAQCLIKPDGRCHLVAVGPCNHAGQGYYPGVGTNTGNIR |
| | | | LIGFECAWPTVNPDGSYNRGQPWPDAQIHTMRDAATAVV |
| | | | RRLGYGSDRVIGHKEYATRAPNVKWDPGNLSMDWFRLQ |
| | | | VAKDLRGEFDPKPADQPRVEVPEPSPTGIPPDFTQQMFL |
| | | | QMSGRWEMLGWQTIVESLAEIRDATIGSSDAGKPGFTLG |
| | | | GRPQTPAAVLAARKTPGTNASATKASARKAPAEKAPAKK |
| | | | ARARKVRAKKASAEKAAARGHHHHHH |
| WP_239340597 | Mycolicibacterium | 167 | MPQAAGWRGDPVWLADVLRAEGLDVAEFAGWRGRGHG |
| | sp. OfavD-34-C | | DFHDIRGVMVHHTGSDFATAASIAYGRPELPGPLSQVHIG |
| | | | RNGTVTVVAAGVAWHAGVGMYPWLPVNMGNWHTIGIEC |
| | | | ANSGTAPWAPHRQNWPEAQYFALVNTCAAISRRLGLPAA |
| | | | RTIGHKEYAGRAQGKWDPGAIDMDRLRLDVARQIGAPAR |
| | | | ATSPRPAVPVGRYADVLLYRGTRGRQVAELQRRLKFAYA |
| | | | SYAGGLEISGEFDVATEEAVREFQRRTPGLHVDGIVGPAT |
| | | | AAALELRLIDVDL |
| WP_239340597 | Mycolicibacterium | 168 | MPQAAGWRGDPVWLADVLRAEGLDVAEFAGWRGRGHG |
| (6X-His) | sp. OfavD-34-C | | DFHDIRGVMVHHTGSDFATAASIAYGRPELPGPLSQVHIG |
| | | | RNGTVTVVAAGVAWHAGVGMYPWLPVNMGNWHTIGIEC |
| | | | ANSGTAPWAPHRQNWPEAQYFALVNTCAAISRRLGLPAA |
| | | | RTIGHKEYAGRAQGKWDPGAIDMDRLRLDVARQIGAPAR |
| | | | ATSPRPAVPVGRYADVLLYRGTRGRQVAELQRRLKFAYA |
| | | | SYAGGLEISGEFDVATEEAVREFQRRTPGLHVDGIVGPAT |
| | | | AAALELRLIDVDLHHHHHH |
| WP_240619086 | Mycobacterium | 169 | MTAAEFAPAVLQAGRDLGISPKGIVIGFATVYVESNWTMY |
| | intracellulare | | ANAKVPDSMNIPHEAVGSDGYSVGLFQQQVVNSGNGW |
| | | | WWGDAATCMDPYKSAQLFFSRLKRLDYNSGAQSPGSYA |
| | | | QAIQQSAFPDRYDQRMADAQALYDQISGGTVTTPPQDPR |
| | | | LTALQKVRPDFNELEFWCDNNQDRAGTKVDLWLIHTQEP |
| | | | GNLTDNDAALHLAQFLKSTEGSGNPVSYHYTIRQARDGG |
| | | | VTVYDIVDTDLASWSVGNSNNRAINACFAGSGVAWSRAD |
| | | | WMTQAKAIDVAAYLAVQDCLKYGIDPNVIPGPQYGQNPP |
| | | | GISDHRYCSNWLKDGNNHDDVGDNFPWDVFSAAVAKYW |
| | | | AAANAGTTPPPADTGGDTTPADPFEAWVAGATDRQLLEY |
| | | | IVEQLGPGDPSWNSKGATLRDFLWSLQSQAKQTAAKAAG |
| | | | KRASRKSR |
| WP_240619086 | Mycobacterium | 170 | MTAAEFAPAVLQAGRDLGISPKGIVIGFATVYVESNWTMY |
| (6X-His) | intracellulare | | ANAKVPDSMNIPHEAVGSDGYSVGLFQQQVVNSGNGW |
| | | | WWGDAATCMDPYKSAQLFFSRLKRLDYNSGAQSPGSYA |
| | | | QAIQQSAFPDRYDQRMADAQALYDQISGGTVTTPPQDPR |
| | | | LTALQKVRPDFNELEFWCDNNQDRAGTKVDLWLIHTQEP |
| | | | GNLTDNDAALHLAQFLKSTEGSGNPVSYHYTIRQARDGG |
| | | | VTVYDIVDTDLASWSVGNSNNRAINACFAGSGVAWSRAD |
| | | | WMTQAKAIDVAAYLAVQDCLKYGIDPNVIPGPQYGQNPP |
| | | | GISDHRYCSNWLKDGNNHDDVGDNFPWDVFSAAVAKYW |
| | | | AAANAGTTPPPADTGGDTTPADPFEAWVAGATDRQLLEY |
| | | | IVEQLGPGDPSWNSKGATLRDFLWSLQSQAKQTAAKAAG |
| | | | KRASRKSRHHHHHH |
| WP_246228822 | Mycolicibacterium | 171 | MSWVSFEPRPMRSREEIARIVHGVSLRRGLDELATAIALM |
| | psychrotolerans | | TIAVESGSNGHWWCPWNAKDSTSRNYQFDSQSDDGFSV |
| | | | GYFQQQNRRPGETPSGSDDWWGPMRSRMTLEDAADQF |
| | | | LARLTHDWRSAADNPALAGRFAQDVQGSGFGERYQQH |
| | | | WDEAWDVLRRALAEAAVPEPTLVAKKSGEVMGFVGDPV |
| | | | WLEDVLREALGDRLVVEPDWEKRGTGGAMGDVWGVMIH |
| | | | HTGNINETVSAIRDGVHQPGGFLPGPLAQCLIKPDGTCHLI |
| | | | AVGPCNHAGRGSYQGVGTDCGNRRLIGFECCWPTPRPE |
| | | | LPGGCDTCEPWSAPLIITMRDATAAVLTKLGYSSERVIGH |
| | | | KEYAGSAQGKWDPGNLSMDWFRGEVQKDLDDAVFPGE |
| | | | QPMPAPEPDPDPVLPPKLLPEPNPRSDRQLLEEIWDQLR |
| | | | GPGGNGWRQLGGNTVVDYLHEIGCFATDLRHQLDELST |
| | | | MLEALTTRGLSTTPNGSTAPRKAAAKKVPPKKIPAKKTPA |
| | | | KKTAASAATRKPHAKKPAKKTS |
| WP_246228822 | Mycolicibacterium | 172 | MSWVSFEPRPMRSREEIARIVHGVSLRRGLDELATAIALM |
| (6X-His) | psychrotolerans | | TIAVESGSNGHWWCPWNAKDSTSRNYQFDSQSDDGFSV |
| | | | GYFQQQNRRPGETPSGSDDWWGPMRSRMTLEDAADQF |
| | | | LARLTHDWRSAADNPALAGRFAQDVQGSGFGERYQQH |
| | | | WDEAWDVLRRALAEAAVPEPTLVAKKSGEVMGFVGDPV |
| | | | WLEDVLREALGDRLVVEPDWEKRGTGGAMGDVWGVMIH |
| | | | HTGNINETVSAIRDGVHQPGGFLPGPLAQCLIKPDGTCHLI |
| | | | AVGPCNHAGRGSYQGVGTDCGNRRLIGFECCWPTPRPE |
| | | | LPGGCDTCEPWSAPLIITMRDATAAVLTKLGYSSERVIGH |
| | | | KEYAGSAQGKWDPGNLSMDWFRGEVQKDLDDAVFPGE |
| | | | QPMPAPEPDPDPVLPPKLLPEPNPRSDRQLLEEIWDQLR |
| | | | GPGGNGWRQLGGNTVVDYLHEIGCFATDLRHQLDELST |
| | | | MLEALTTRGLSTTPNGSTAPRKAAAKKVPPKKIPAKKTPA |
| | | | KKTAASAATRKPHAKKPAKKTSHHHHHH |
| WP_247055243 | Mycolicibacterium | 173 | MADRTVYGNTHSENGWRMVDQGSCVWVKVPGTNVTLQI |
| | sp. F2034L | | REGQPAAIMGAFAADYNAHVEPLRDADSACWTATNSVGT |
| | | | SNHLSGTGMDLNWNGPDGRTFRLGISKERAYPGDKARKL |
| | | | DELLTFYEGIIFCGGEWSIRDWMHMQMGGNTYGARNVER |
| | | | VNDFIRRKIRPDGFSTFKRGGATAPPALPAPPPAGGNQVD |
| | | | VLARATGISTAKATEILPLVVFALGKCNCTNPRRIAAALAQ |
| | | | WVIESGHFVYTEEIASGPESQERWKYKGRTWIQLTWLEN |
| | | | YLGFSRWVHSLGLVPTPTYFGDRPRELADLKWAALGPAY |
| | | | WWAIKYPQINEYADRGDIDNVSKWVNAPAWVDNPNKHA |
| | | | NHEKERRDAYNRALALGDRLLTLIATPEPLDELEAIMATEI |
| | | | DSWSIYANPGEPKIPVVNLIAAIDAKIHRDLTDEDARAGDP |
| | | | EAIRRVARTAAGLGRVKTPGAIQHAVRVLNEIQATNPQYIL |
| | | | AAIPGKA |
| WP_247055243 | Mycolicibacterium | 174 | MADRTVYGNTHSENGWRMVDQGSCVWVKVPGTNVTLQI |
| (6X-His) | sp. F2034L | | REGQPAAIMGAFAADYNAHVEPLRDADSACWTATNSVGT |
| | | | SNHLSGTGMDLNWNGPDGRTFRLGISKERAYPGDKARKL |
| | | | DELLTFYEGIIFCGGEWSIRDWMHMQMGGNTYGARNVER |
| | | | VNDFIRRKIRPDGFSTFKRGGATAPPALPAPPPAGGNQVD |
| | | | VLARATGISTAKATEILPLVVFALGKCNCTNPRRIAAALAQ |
| | | | WIESGHFVYTEEIASGPESQERWKYKGRTWIQLTWLEN |
| | | | YLGFSRWVHSLGLVPTPTYFGDRPRELADLKWAALGPAY |
| | | | WWAIKYPQINEYADRGDIDNVSKWVNAPAWVDNPNKHA |
| | | | NHEKERRDAYNRALALGDRLLTLIATPEPLDELEAIMATEI |
| | | | DSWSIYANPGEPKIPVVNLIAAIDAKIHRDLTDEDARAGDP |
| | | | EAIRRVARTAAGLGRVKTPGAIQHAVRVLNEIQATNPQYIL |
| | | | AAIPGKAHHHHHH |
| WP_265125046 | Hoyosella sp. YIM | 175 | MAWTGNVLWLAEALRAEGLRVIEHPGWQQRGHGEYRDI |
| | 151337 | | RGVMCHHTAGGGPNDWQVVLNGRPDLPGPLSNLVLEKD |
| | | | GTYRVIAAGVCWHAGRGSWPGWPTDNANWHVIGIEAVS |
| | | | RGVPDAQGRYDWTQEQLDAYVRGCAVIAHRAGFSVRDV |
| | | | VGHKEYSSEGKIDPAGIDMDDFRAQVQQVLDRMRAPRDL |
| | | | DAFPLPPGYYYGPLEGPTQSISGLHPSERPEWREALKRW |
| | | | QRAQRVPETGVWDAETITAAKAVQSANGWNPDGLIGPGT |
| | | | WAAGLRGTVPPAEPEPAPVPLPEPEPVPEPAPVPEPEPA |
| | | | PGPEPVGASPRTVLREVVRWLRDRIPVGK |
| WP_265125046 | Hoyosella sp. YIM | 176 | MAWTGNVLWLAEALRAEGLRVIEHPGWQQRGHGEYRDI |
| (6X-His) | 151337 | | RGVMCHHTAGGGPNDWQVVLNGRPDLPGPLSNLVLEKD |
| | | | GTYRVIAAGVCWHAGRGSWPGWPTDNANWHVIGIEAVS |
| | | | RGVPDAQGRYDWTQEQLDAYVRGCAVIAHRAGFSVRDV |
| | | | VGHKEYSSEGKIDPAGIDMDDFRAQVQQVLDRMRAPRDL |
| | | | DAFPLPPGYYYGPLEGPTQSISGLHPSERPEWREALKRW |
| | | | QRAQRVPETGVWDAETITAAKAVQSANGWNPDGLIGPGT |
| | | | WAAGLRGTVPPAEPEPAPVPLPEPEPVPEPAPVPEPEPA |
| | | | PGPEPVGASPRTVLREVVRWLRDRIPVGKHHHHHH |
| AAD17596 | Mycobacteriophage | 177 | MSFTRFLQDDPLLTREQVMAELIRVADELNMPDKRGACVI |
| | TM4 | | AGMTISQEVGVKDNDPPFERRFWCPANRADPESFNYPH |
| | | | DSESNDGRSVGYFQQQKGPNGELWWGTTASEMNLHSA |
| | | | ATQFMTRLKAAGYNASNAQAANDSAQAIQRSGVPQAYKQ |
| | | | WWDDINRLYDKVKGSGGGPAPAPKPPQSGPWTGDPVW |
| | | | LADVLRAEGLNVVELPGWLDRGHGDMGRLWGVVCHHTG |
| | | | SDNTPSSEIAFHPSLGLCSQIHLARNGTVTLCGVGIAWHA |
| | | | GVGSYPGLPEDNANAVTIGIEAQNSGTYDGAPHRTNWPD |
| | | | AQYDAYVKCCAAICRRLGVRADHVISHKEWAGRKQGKW |
| | | | DPGAIDMNIFRADVQRRIDAHQPNGEDDFMAALSADEQR |
| | | | EVLNLLRVLADRRFVSRSPFRHLGEGPSETVAGFGLNTD |
| | | | GLNHAQYTIELARLGDPTHLALLREVASAEGDSRYPDRQY |
| | | | DAKLAKRVLAEIEGAATAPAKPSTPSAPTEPAPEAPTPPVK |
| | | | AACALSAAGCVVAGSTSGGGCALSTDGTGKCVVTAATDG |
| | | | GAA |
| AAD17596 | Mycobacteriophage | 178 | MSFTRFLQDDPLLTREQVMAELIRVADELNMPDKRGACVI |
| (6X-His) | TM4 | | AGMTISQEVGVKDNDPPFERRFWCPANRADPESFNYPH |
| | | | DSESNDGRSVGYFQQQKGPNGELWWGTTASEMNLHSA |
| | | | ATQFMTRLKAAGYNASNAQAANDSAQAIQRSGVPQAYKQ |
| | | | WWDDINRLYDKVKGSGGGPAPAPKPPQSGPWTGDPVW |
| | | | LADVLRAEGLNVVELPGWLDRGHGDMGRLWGVVCHHTG |
| | | | SDNTPSSEIAFHPSLGLCSQIHLARNGTVTLCGVGIAWHA |
| | | | GVGSYPGLPEDNANAVTIGIEAQNSGTYDGAPHRTNWPD |
| | | | AQYDAYVKCCAAICRRLGVRADHVISHKEWAGRKQGKW |
| | | | DPGAIDMNIFRADVQRRIDAHQPNGEDDFMAALSADEQR |
| | | | EVLNLLRVLADRRFVSRSPFRHLGEGPSETVAGFGLNTD |
| | | | GLNHAQYTIELARLGDPTHLALLREVASAEGDSRYPDRQY |
| | | | DAKLAKRVLAEIEGAATAPAKPSTPSAPTEPAPEAPTPPVK |
| | | | AACALSAAGCVVAGSTSGGGCALSTDGTGKCVVTAATDG |
| | | | GAAHHHHHH |
| YP_009018718 | Mycobacteriophage | 179 | MLTVESFAAAMGNSLSVDRYRQLFPAAVESMVACGCTTV |
| | DS6A | | NRAAMWLAQVGHESGGLRWMEELASGAAYEWRSDLGN |
| | | | TQAGDGVRFKGRGPIQITGRYNYRKVSEWAHAQGIVPTP |
| | | | TYFVDNPTQLASDQYGFIGVSWYWQHGGPRPGQINGFA |
| | | | DAGDILSGSRCVNGWVTTPNGMPDRTERWNRCRAMGD |
| | | | QILPAGAGTDNGGDGMAWTGDPVWLEDVLRPVLGDRLR |
| | | | VLPSWQMYGHGDFKDIRGVMVHHTGNARETAESIRKGR |
| | | | PDLRGPLSNIHIAPDGTVTLVAAGVCWHAGAGSYPWLPT |
| | | | NNANWHMIGIECAWPTIRPNGTYDEREPWPDAQIIAMRDT |
| | | | CAALTKRLGWDASRVIGHKEYAGASQGKWDPGNLDMG |
| | | | WFRGEVAKAMRGEFDPKAPAPAPNPPADNDNGGFLMAL |
| | | | NDKQQHDLYNAVMTIAAVAADIQTQLRGPGLRGWPQLGR |
| | | | NANGEALTLVDAVGVIKAIADREAGR |
| YP_009018718 | Mycobacteriophage | 180 | MLTVESFAAAMGNSLSVDRYRQLFPAAVESMVACGCTTV |
| (6X- | DS6A | | NRAAMWLAQVGHESGGLRWMEELASGAAYEWRSDLGN |
| His) | | | TQAGDGVRFKGRGPIQITGRYNYRKVSEWAHAQGIVPTP |
| | | | TYFVDNPTQLASDQYGFIGVSWYWQHGGPRPGQINGFA |
| | | | DAGDILSGSRCVNGWVTTPNGMPDRTERWNRCRAMGD |
| | | | QILPAGAGTDNGGDGMAWTGDPVWLEDVLRPVLGDRLR |
| | | | VLPSWQMYGHGDFKDIRGVMVHHTGNARETAESIRKGR |
| | | | PDLRGPLSNIHIAPDGTVTLVAAGVCWHAGAGSYPWLPT |
| | | | NNANWHMIGIECAWPTIRPNGTYDEREPWPDAQIIAMRDT |
| | | | CAALTKRLGWDASRVIGHKEYAGASQGKWDPGNLDMG |
| | | | WFRGEVAKAMRGEFDPKAPAPAPNPPADNDNGGFLMAL |
| | | | NDKQQHDLYNAVMTIAAVAADIQTQLRGPGLRGWPQLGR |
| | | | NANGEALTLVDAVGVIKAIADREAGRHHHHHH |
| QRJ69315 | Mycobacteriophage | 181 | MSFRTVNGNTHTEDGWRCCNRDECDIVRIPELYLVDTAP |
| | phiT45-1 | | LRKGAPLTILGAWLYWYDRNVEEITSPVWGWSATNDVLG |
| | | | TPGRNDGSNHLSGTAVDVMAPKYPWQQYTMNAATQAKV |
| | | | RKGLALFEGSVFWGRDWSRPDEMHYQMAWPEGDKRND |
| | | | AFAAKLRAGYLGIYAPAQPPAPVQKRFPQDLSDRELLEYIA |
| | | | EQLGPGHPDWASKGMTLRDKVWSK |
| QRJ69315 | Mycobacteriophage | 182 | MSFRTVNGNTHTEDGWRCCNRDECDIVRIPELYLVDTAP |
| (6X-His) | phiT45-1 | | LRKGAPLTILGAWLYWYDRNVEEITSPVWGWSATNDVLG |
| | | | TPGRNDGSNHLSGTAVDVMAPKYPWQQYTMNAATQAKV |
| | | | RKGLALFEGSVFWGRDWSRPDEMHYQMAWPEGDKRND |
| | | | AFAAKLRAGYLGIYAPAQPPAPVQKRFPQDLSDRELLEYIA |
| | | | EQLGPGHPDWASKGMTLRDKVWSKHHHHHH |

Additional sequences were identified that may be useful in the compositions and methods described herein. In some embodiments, the composition includes a Lysin B that includes an amino acid sequence having at least 85% (e.g., at least 90%, 95%, 97%, 99%, or 100%) sequence identity to of any one of SEQ ID NOs: 183-241 as shown in Table 3 below.

TABLE 3
Lysin B Sequences

| | Phage/Host | SEQ | |
|---|---|---|---|
| NCBI ID | Species | ID NO: | Sequence |
| NP_046827.1 | D29 | 183 | MSKPWLFTVHGTGQPDPLGPGLPADTARDVLDIYRWQPI |
| | | | GNYPAAAFPMWPSVEKGVAELILQIELKLDADPYADFAMA |
| | | | GYSQGAIVVGQVLKHHILPPTGRLHRFLHRLKKVIFWGNP |
| | | | MRQKGFAHSDEWIHPVAAPDTLGILEDRLENLEQYGFEV |
| | | | RDYAHDGDMYASIKEDDLHEYEVAIGRIVMKASGFIGGRD |
| | | | SVVAQLIELGQRPITEGIALAGAIIDALTFFARSRMGDKWP |
| | | | HLYNRYPAVEFLRQI |
| NP_046827.1 | D29 | 184 | MSKPWLFTVHGTGQPDPLGPGLPADTARDVLDIYRWQPI |
| (6X-His) | | | GNYPAAAFPMWPSVEKGVAELILQIELKLDADPYADFAMA |
| | | | GYSQGAIVVGQVLKHHILPPTGRLHRFLHRLKKVIFWGNP |
| | | | MRQKGFAHSDEWIHPVAAPDTLGILEDRLENLEQYGFEV |
| | | | RDYAHDGDMYASIKEDDLHEYEVAIGRIVMKASGFIGGRD |
| | | | SVVAQLIELGQRPITEGIALAGAIIDALTFFARSRMGDKWP |
| | | | HLYNRYPAVEFLRQIHHHHHH |
| QGJ93185 | TyDawg | 185 | MWIGWQEGMSGRPVLAAKGELRAKFSYGKNLDLTEHFG |
| | | | PDLTEALKTFQRNKGGLRTDGVLDYATQKALGVLEALKP |
| | | | WVFTVAGTGAGWDAGYPAEVARAVLDLYRWQGVNYPAK |
| | | | PFPMGSSVDAGIAELKKLLRERMDRYPAARFVLIGYSQGA |
| | | | IVTSMVWQNFIKGSDLEARIIGSITYGNPCRELHVANGNVA |
| | | | EGIPVPEGRGISDFRLRSTPSWWYDFAHGANSRFGRDIY |
| | | | TDTPDDDAGEMMTAIYRLVQDLKNIIVGTDSLIEQVGEMF |
| | | | QRPITEVGAAMWAIFLGGQFVATRPYPTMPHINYDINPAV |
| | | | AILRRIAA |
| YP_010049701 | Typha | 186 | MAWSPPSKVGDIAPVVAAAKQKIQKFQYGRDNVPSTDTS |
| | | | EVYTEGLLTAMLEFQRRVHNEVLAGQRETPDVPTDGTID |
| | | | WATQVQLGIIERQKPTIPTVGPRHPAIVFRGTGGIIGQDLV |
| | | | SLVCQHCADLVEEINPDWPATMGGIPVGTAGNISDPSMW |
| | | | RGVQIAVADAQRRFLDILAKRPAVKIVIGGYSAGAVAAAIFK |
| | | | QWIMDNFPDHYLCSFSFGDPTRPVGGAFFAAPVIPWGRG |
| | | | VASVHYGDPKDWRHCWLTAEGDMYGQVPQGVTGDIMD |
| | | | DAFDMVTRVELSNILETAQAIIPRIPEVAEKAGIGLPSVLGA |
| | | | LAGGIPGLLGLGLPWALGAISGLVGTGNPDLLTGSAAAAK |
| | | | AAQIALTFAAQGTAPHIEYHMREVWPGQTYLGLAIQHVRD |
| | | | YASATPATA |
| YP_008051660 | Dumbo | 187 | MTTYGELKALRLGVKYVRHTLFTVAGTWADMWSGYPAD |
| | | | VARLVDEDLFRWQPVWYPASFGPVGNPLGRSYQESVQD |
| | | | GVKELVRLINATPGTFALVGYSQGAEVVSRVLLEILFGSLR |
| | | | HRLKDFIGGGCFGNPYRAKGVSYPGSGLPTSGHGIAPVN |
| | | | LAPDILPAEMWEEWWNEGDLYAQNLDGKSGEIITSFYDIL |
| | | | TKLQFHDMLGLAVNMFKALSNDKGIIAQVMRVLAVPLPGVI |
| | | | DAGRAVVYAGTFAVQGTRPHITLAETGRVARAVWHLNRI |
| | | | GAKTLARAS |
| AGT20697 | Dylan | 188 | MRIGGEYVGLGLGDASEEIGRIRDFMRRKFRSYAGNLPDT |
| | | | RNALGLPLFDEPMTVAVTEMQGRYLQSGELRDGLFIFGIIN |
| | | | AETKYVMGYLERPPGPDTRPLFITVCGTGVPWWVGPDAD |
| | | | TARACEDKLFWQPIGYPAKNFPMGKSIAAGIDQCHVQFN |
| | | | RADPGFMHRQRIERNGVVLGGYSQGAVVVSELWENNIKP |
| | | | ANGSLHWAKDYVVKAVTWGNPNREVGAVWPDYGGSPM |
| | | | ASLTSQGVSSTGMRDTPSWWRNYAHAGDLYAAAEPGDS |
| | | | QQDKNAIWQIIRDLNFFTGTDSLLAQAIELSQMPIPRTIAAF |
| | | | KALIDAGMFFAKGTGPHVDYGIGPAIEYLRS |
| NP_817485 | Cjw1 | 189 | MTTYGELKALRLGVKYVRHTLFTVAGTWADMWSGYPAD |
| | | | VARLVDEDLFRWQPVWYPASFGPVGNPLGRSYQESVQD |
| | | | GVKELIRLINATPGTFALVGYSQGAEVVSRVLLEILFGSLR |
| | | | HRLKDFIGGGCFGNPYRAKGVSYPGSGLPTSGHGIAPVN |
| | | | LAPDILPAEMWEEWWNEGDLYAQNLDGKSGEIITSFYDIL |
| | | | TKLQFHDMLGLAVNMFKALSNDKGIIAQVMRVLAVPLPGVI |
| | | | DAGRAAVYAGTFAVQGTRPHITLAETGRVARAVWHLNRI |
| | | | GAKTLARAS |
| YP_009011947 | Courthouse | 190 | MPLRLGDRNESVRQWRIKMNAWFGPLYTRLHGPLPMDT |
| | | | DEFGPRAKSWQEEYERRTNQPVDGEVSDNDLRALNVPV |
| | | | PTKWVIFTVAGTGARWDQTYPYDLGRWQDQNRVILQPIG |
| | | | YPAATFPMGPSVDKGEAELVNQMRRHLDANPSLNFILVG |
| | | | YSQGAIVTSRVLRRMMSGDLAHYYNRCIAGVTFGNPMRE |
| | | | RGHFVGVNDPGGQGLDPKPLVDTPSWWYDYAARGDIYS |
| | | | SGPGNNDRQAAEHMTSIYLAVMGKFILGNDALVSQVIELF |
| | | | TNPFAEVPAVVKAIASGIGFVTSNPPTAAHIEYHIRECVPG |
| | | | VTYFDHAMGYVRQVISANKRIA |
| QDF19322 | Cracklewink | 191 | MATWDSILDYLRGDPCKVGYFGVPGTWSPWDAGYQADV |
| | | | GRLINSAWFYWQGVGYVAAFGPVNGPITNPSYAESVQEG |
| | | | VDETVRLILARPGPIVLGGYSQGAEVVYWVAREFLTGGRL |
| | | | AHRRGDLLLIVTFGNPCRAKGHGVNQATAHGWGISRKPP |
| | | | LTELLPIWLDYALPGDMYCCADDDTYLAIGYAALTKLQLHD |
| | | | PWQLVQAMLALIQSDEFADALAELLDPLFPGLATTLGELT |
| | | | GMDGAALLANRKPVGGGVLGGLMSGQLLTATPAGGNLL |
| | | | GGLIGAGTGMLGGLLDGVIPGGLPPIVGGLLGGGSGPGG |
| | | | TPTGMYKLGKTFAALLNFATTNDHGHYHDTPAFAGTNAV |
| | | | AHAVGEVNRLAA |
| YP_009837106 | Cuke | 192 | MSDRIFFLWLPGTWEVDSLKAENPSATNFVGLGKWLVDD |
| | | | RFPDRLPLNIFEPIMLCPPDYMASFGPIPAAGSSIFSAWNH |
| | | | LSYKESVLDAVEHSVDVILKLPTDRPIIIGGYSQGAEVAERL |
| | | | KAEFLPGGRLSSYFLLAHYTFGNPGRPQGVTFPNGNTLP |
| | | | WGGISNLNIPTPQGTFYRSYAFYDDMYANANPKSYLFEFY |
| | | | DGLTDLQFHDPFKAVKDVVGVVTKSDLMILAGAQPTNPF |
| | | | WVVTHIPQFIDISTKAVNSLDALARFASSGAHGHYGDWEII |
| | | | PGFTPIFHCIRSAKYAAKGIGYAVPGI |
| NP_039676 | L5 | 193 | MSKPWLFTVHGTGQPDPLGPGLPADTARDVLDIYRWQPI |
| | | | GNYPAAAFPMWPSVEKGVAELILQIELKLDADPYADFALA |
| | | | GYSQGAIVVGQVLKHHIINPRGRLHRFLHRLRKVIFWGNP |
| | | | MRQKGFAHTDEWIHQVAASDTMGILEDRLENLEQYGFEV |
| | | | RDYAHDGDMYASIKEDDMHEYEVAIGRIVMSARRFIGGKD |
| | | | SVIAQLIELGQRPIWEGIAMARAIIDALTFFAKSTQGPSWPH |
| | | | LYNRFPAVEFLRRI |
| UOW93367 | Labelle | 194 | MKGLDGSYIGIGLGDVSSEVLKIKQFINRKFSRFRLLETEIY |
| | | | DAAMEAAVRELQTIYKNNGTLTDPFIPGVVNLATKYAIGYL |
| | | | KKDVILPIHFSVEGHMSDMWIGPAAYVGEVLRAENRALHF |
| | | | PTGYDNRALPFNNKSGVEQLVQRVGATEFFIDGKVIKFPP |
| | | | GTPWTASAFSQGAMIWCDFYRQYLMPGKPLHWRLKDLR |
| | | | AVICCGNPDREKGVCVDWIPDKPDPDRQGIMDDENRMV |
| | | | NTPWYWLELARKGDMYTDNESSGERGLNKTAIAKIITQNK |
| | | | WSGGPAGLLARVTDLLVNPIDDVIPITLALYDAIRFGAGGIR |
| | | | AHGGYDMEPAVQFCRERLAA |
| YP_010051699 | Lemuria | 195 | MAYEAPRKVGDTHKLIPAAKAKLAGNSYGKAIGADRSPTY |
| | | | TAAFGAALVQYGKNVHDAVAKGKRRTPDVNTAGVFDWAI |
| | | | QDQMELETKPAAPEAQPLPKGRAIGYVWRGTGGIVGQDL |
| | | | VYQVLQQCSDLVELVDPPWAATMGGIPVGTAGGVGDPS |
| | | | MWSAVQETVAWTQADFIRRRAADPKVRVVLGGYSAGAIA |
| | | | CAIFKAWLLENYPENYVCSYSFGDPTRPFGGGFFGMPAP |
| | | | WGRGISTLSYGDPKDYRHCWLTHEDDMYAQIPGGVVGDI |
| | | | MDDVYEEVTRFAFRDIMQVAMRMMTALPKIAEKAGISLPA |
| | | | VFASLAAGPAGLITFALPMLLSSLGGLIPGSTPDDQLTGTP |
| | | | AAAKAATIGLKFLFAGTRPHTVYHLDPAWPGGPTFVQLAV |
| | | | MHVRDWASRPA |
| AXF51531 | Constella | 196 | MSLWPEIRAMLNGEFYVPAGTIISANGTGVPDGAGPGFG |
| | | | ADLARELERVAEGMWIWRWIDYPAAVFPMRPSINVLREN |
| | | | LKAMIRATPGKLVLTAYSQSAVAFAYVWRDDILNPNGELH |
| | | | DRLDDIEAVILYGNPVRAPGIAYGNELGGQPAPGKLNGHV |
| | | | TGGIAGPDCLKPEECLHPVTGKRVVLDFANAGDLYAAAPV |
| | | | GETPWVEETEVGHNETMIYEAVMDFNGKDILAFAKEIAQIL |
| | | | VLPLTQVVPLVQAIINGLSFLVKGPKAPHWTYDIRPAVDHL |
| | | | VRRGNELR |
| QXN72979 | Phillis | 197 | MSLDNHHIEELEQALRDVKRIGVQTNPLLAPSPAHVTGPT |
| | | | WQRTKDGKFYLPEKTLGYQILYWMSQYLLIPGGPDAGQP |
| | | | FKPTREQGRFILWWYAVDEFGRFVYRNGLLRRLKGWGK |
| | | | DPLAGALALAELCGPVAFSHFDTDGNPIGKRKPAAWIQVA |
| | | | AVSQDQTRNTFSLFPVLASDRLKEEFKLDFNKTIVYAKTIG |
| | | | GVIEAATSNPLTLEGKRPTFVIKNETQWWVETNNGHSMS |
| | | | EVIAGNVDKAAYGGCRSLAICNGHIPGQDSDAERDYDAHL |
| | | | DVLAGKAIDTGFLYDALEAPADTPLSEIPSPIEDPEGFEEGI |
| | | | AKLREGLIIARGDAVWLDLDTIIASMLDKRRPVTESRRKFL |
| | | | NQINAHEDSWIAPHEWNACHPEEGVPPLAKGDRITLGFD |
| | | | GSKSNDHTALVACRVEDGCLFLIKTWDPEKYPGGEVPRD |
| | | | EVDATVRSMKAGYDVVAFRADVKEFEAYVDQWGKDFRK |
| | | | TVQVNASPGNPIAFDMRGNQKKFALDCERFLDAVLERELF |
| | | | HDDNPILRQHVFNARRHPTNYDAIAIRKASKDSSKKIDAAV |
| | | | CAVLAFGARQDFLMSKKNRTRRAAVIM |
| YP_009032423 | ZoeJ | 198 | MATVDYRLGMSGDQVKVIKRLLIDGYYYVRERYPRMTAA |
| | | | SDVYDVYTQASIVEFQFRAGLPVTGVADYATQVRLGAVVP |
| | | | PPPPRQRIMVLTFSGTSADMWTGYPADVARALDPSIFYW |
| | | | QPVCYGPNGIPAIFPMGSSAKSGEVEGLRLLDEKAGDFDY |
| | | | IVLIGYSQGALPASRLMRRILSGDLQRFKAKLIAGVTFGNP |
| | | | MREKGHTFPGGADPGGHGLDPQCLVNTPDWWHDYAAK |
| | | | GDIYTVGSGGNDETANADMTFIYQLVQGDILAMLFGTGNP |
| | | | LDILGALGGGLLGGFGGGGLLGGNKGGLQLPSGLVLPGV |
| | | | GLGQGGALTQRQRGLVEAVLALLANPFAEVPPAVKAIVSG |
| | | | IGFVATNPPTAPHIEYHIREAAPGVTYFQHAIDYLRQVGAS |
| | | | VAARAA |
| YP_009950929 | Rando14 | 199 | MSEPMLLTAQGTGVDMYTGYPHDLGLRLRDEGLVELQPI |
| | | | GKYPARMFPMGPSVKIGVDEGVDLVLKAEARPSREVPDG |
| | | | YLLSGYSQGAWLVSDLLDEFRGGRLKHLRHKLAGGATFG |
| | | | NPRRGLDENGGRGIADKLIVDTPDTWVDEFDPGDIYANVP |
| | | | NNDIGEDMTAIFKLVRLNGISDVIDLRSALGLGGVLGGIMP |
| | | | GGNQLASMLGGLLGGGEQNNITEQIVEMLKSPLREFPAA |
| | | | VQAIVKAAVFFGRKPATAPHIEYHIRTVTPGGPTYFEHAVS |
| | | | HMRAVAA |
| YP_009009685 | Jolie2 | 200 | MAYQAPRKVGDTHPLIPAAKRKLAGNSYGKAIGDDRSNV |
| | | | YTAAFGAALVQYGKNVHDAVLRGKRRSPDVNTVGVFDW |
| | | | AVQDQMELEEKPAAPVAQPLPKDRAIGYVWRGTGGIVGQ |
| | | | DIVYEVIRQCSDLVELVDPPWAATMGGIPVGTAGGIGDPS |
| | | | MWQAVQETVAWTQADFIARRNANPKIRVVLGGYSAGAIA |
| | | | CAIFKAWLLENFPENYVCSYSFGDPTRPKGGGFFGMPAP |
| | | | WGRGISTLSYGDPNDYRHCWLTHEEDMYAQIPGGVAGDI |
| | | | MDDVYEEVTRFAFRDIMQVAMRMMTALPKIAEKAGISLPA |
| | | | VFASLAAGPVGLITFALPLLMGSIGSLINQDQDKDSLTGTP |
| | | | AAAAAAMIGLKFLFAGTRPHIVYHLDPAWPGGPTFVQLAV |
| | | | MHVRDWASRPAA |
| QSM01088 | Nanosmite | 201 | MVWIGWQEGMDGLPVLAAKGELRAKFSYGKNLDTTTHF |
| | | | GPDLTAALQTFQRNKGGLRTDGVLDYATQKALGVLEALK |
| | | | PWAFTVAGTGAGWDAGYPADVARAVLDLYRWQGVNYP |
| | | | AAPFPMGDSVNAGIAELIKLLKQRLDRYPAAKFVLIGYSQG |
| | | | AIVTSMVWKRYIKGTPLEDRIIGSITYGNPCRELGVANGNV |
| | | | AQGIPVPEGRGIADDLLESTPSWWYDFAHGANSPFGRDI |
| | | | YTDTPDDDAGEMMTAIYRLVQSLKNILFGTDSLLEQVGEL |
| | | | FKRPLAEVYAAFRAIVYGGQFFITRPYPTMPHITYDINPAIQ |
| | | | ILRRIAKN |
| URM86476 | Hilltopfarm | 202 | MAWAPPSKVGDIAPVVAEAKKKIQKFQYGRDNVPSTDTS |
| | | | EVYTEGLMTAMLEFQRRVHDEVLAGKRETPDVPTDGTID |
| | | | WATQVQLGIIERQKPTIPTVGPRHPAIVFRGTGGIIGQDLV |
| | | | SLVCQHCADLVEEINPDWPATMGGIPVGTAGNISDPSMW |
| | | | RGVQIAVADAQRRFLDILAKRPAVKIVIGGYSAGAVAAAIFK |
| | | | QWIMDNFPDHYLCSFSFGDPTRPVGGAFFAAPVIPWGRG |
| | | | VASVHYGDPKDWRHCWLTAEGDMYGQVPQGVTGDIMD |
| | | | DAFDMVTRVELSNILETAQAIIPRIPEVAEKAGIGLPSVLGA |
| | | | LAGGIPGLLGLGLPWALGAISGLVGTGNPDLLTGSAAAAK |
| | | | AAQIALTFAAQGTAPHIEYHMREVWPGQTYLGLAIQHVRD |
| | | | YASATPATA |
| YP_009841702 | Fowlmouth | 203 | MTDRIFFLWLPGTWEVDMFKNENPSATNFVGLGKWLVDD |
| | | | RFPDRLPLSIFEPIMLCPPDYMASFGPIPAAGSSIFSAWNH |
| | | | LSYKESVMDAVEHSVQIILKLPVSRPIVIAGYSQGAEVAERI |
| | | | KAEFLPGGRLERYFLLSHYTFGNPGRPQGVTFPNGNILP |
| | | | WGGISNLNIPTPQNTYYRSYAFYDDMYANANPKSYLFQFY |
| | | | DGLTDLQFHDPFKAVKDVLGVVTKSDLMILAGAQPTNPLW |
| | | | IMMNIPKFIDIGTRAANSLDALARFASSGAHGHYHDWEIIP |
| | | | GFTPIFHCIRSAKYAAKGIGYAVPGI |
| YP_010013648 | Aziz | 204 | MAWIGWQEGMEGLPVLAAIGELRAKFSYGKQLELTQKFT |
| | | | PELTAAVQTFQVKKGGLRTDGVLDYDTQKALGVLEALKP |
| | | | WVFTVAGTGAFWDAGYPADIARAVADLFRWQGVNYPAK |
| | | | PFPMGDSVDAGIAELLKLLKQRLDRYPAAKFALIGYSQGAI |
| | | | VTSMVWKRYIKGNEDLEKRIIGSITFGNPCRELGVANGNV |
| | | | AEGIPIPEGRGIGDDRLQSTPEWWYDFAHGANSPFGRDIY |
| | | | TDTPDDDTGEMMTAVYRAVQKLANLWSGVDSILEQVGEII |
| | | | KRPIPEIYAAFRAVIYGGQFVTTQPWATYPHTNYNIDTAVA |
| | | | FLRRIAKN |
| ASR85818 | Guillsminger | 205 | MSKPMLLTGQGTGADMFTGYPHDLGVRLRDEDRVELQPI |
| | | | GKYPASMFPMGPSVKIGVDEGVDLVLRAEARPAREVPDG |
| | | | YLLCGYSQGAWLVSDLLDEFRGGRLKHVAHKLAGGVTFG |
| | | | NPRRELDQSGGRGIADTLIVGTPDTWVDEFDPGDIYANVP |
| | | | NNDVGEDMTAIFKLVRLNGISDVVDLRSGLDLAGVLGGLL |
| | | | VPGVGQLGGLAGGLGALFGGAQEQNTIVEQVVEMLKSPL |
| | | | REFPAAVMAIVKAAVFFGRKPATAPHIEYHIRTVTPGGPTY |
| | | | FEHAVNHMRAMAA |
| AVR77139 | Midas2 | 206 | MELTEQDIENLEKAVAEAKRIEPVTDPELAPSPPHIIGPTW |
| | | | QRRADGRFYLPEKTLGYQILMWMSEYLLIPGGPKAGEPF |
| | | | KPTREQGRFILWWYAVDEFGRFVYRNGLLRRLKGWGKD |
| | | | PLAGALALAELCGPVMFSHFDSEGNPVGKRKPSAWIQVA |
| | | | AVSQDQTRNTFSLFPVLASDRLKEEFKLDFNKTIVYAKAIG |
| | | | GVIEAATSNPLTLEGKRPTFVIKNETQWWIETNNGHAMSE |
| | | | VIAGNVDKSAYGVCRSLAICNGHIPGQDSDAERDYDAYM |
| | | | DVLAGKAIDTGFLYDALEAPADTPLSEIPPPTEDPEGFEKG |
| | | | IAQLREGLKIARGDAVWLDLDTIIASMLDKRRPVTESRRKF |
| | | | LNQINAHEDSWIAPHEWDRLALTEDLFKLKKGDRIALGFD |
| | | | GSKANDHTALVACRIEDGMLFLIKHWNPEHHGGEVPRDD |
| | | | VDATVRSCFERYDVVAFRADVKEFEAYVDQWGKDFRKK |
| | | | VQVNASAGNPVAFDMRGQQKRFALDCERFLDAVLEREV |
| | | | YHDGNPVLRQHVLNARRHPTNYDAIAIRKASKDSSKKIDA |
| | | | AVCAVLAFGGRQDFLMSKKNRSRRAAVIM |
| AYN57203 | BoostSeason | 207 | MAWVGWQLGMQGEQVKVIQQKLIGKYQWVRDRYPRLTA |
| | | | SGVYDVFTQAAIVEFQFRVGLPVTGIADYATQVRLGAVVP |
| | | | APPPRQRIMVLTFSGTSADMWTGYPADVARALDPSIFYW |
| | | | QPVCYGPNGIPAIFPMGSSAKSGEVEGLRLLDEKARDFDY |
| | | | IVLIGYSQGALPASRLMRRILSGDLQRFKSKLIAGVTFGNP |
| | | | MREKGHTFPGGADPGGHGLDPQCLVNTPDWWHDYAAK |
| | | | GDIYTVGSGSNDEKANADMTFIYQLVQGDILGMMFGTGN |
| | | | PLDILGLLGGLGGGLLGGLGGGLLGGGKGGLQLPSGLVL |
| | | | PGVGLGQGGALTDHQRGLVEAVLALLANPFAEVPAAVKAI |
| | | | VSGVGFIATNPPTAPHIEYHIREAAPGVTYFQHAIDYLRQV |
| | | | GASVAARAA |
| YP_009303183 | Bipper | 208 | MATWDSILDYLRGDPCKVGYFGVPGTWSPWDAGYQADV |
| | | | GRLINSAWFYWQGVGYVAAFGPVNGPITNPSYAESVQEG |
| | | | VDETVRLILARPGPIVLGGYSQGAEVVYWVAREFLTGGRL |
| | | | AHRRGDLLLIVTFGNPCRAKGHGVNQATAHGWGISRKPP |
| | | | LTELLPIWLDYALPGDMYCCADDDTYLAIGYAALTKLQLHD |
| | | | PWQLVQAMLALIQSDEFADALAELLDPLFPGLATTLGELT |
| | | | GMDGAALLANRKPVGGGVLGGLMSGQLLTTTPAGGNLL |
| | | | GGLIGAGTGVLGGLLDGVIPGGLPPIVGGLLGGGSGPGGT |
| | | | PTGMYKLGKTFAALLNFATTNDHGHYHDTPAFAGTNAVA |
| | | | HAVGEVNRLAA |
| AKU45330 | Madruga | 209 | MKGLDGSYIGIGLGDVSSEVLKIKQFINRKFSRFKLLETEIY |
| | | | DAAMEAAVRELQTIYKNNGTLTDPFIPGVVNLATKYAIGYL |
| | | | KKDVILPIHFSVEGHMSDMWIGPAAYVGEVLRAENRALHF |
| | | | PTGYDNRALPFNNKSGVEQLVQRVGATEFFIDGKVIKFPP |
| | | | GTPWSASAFSQGAMIWCDFYRQYLMPGKPLHWRLKDLR |
| | | | AVICCGNPDREKGVCVDWIPDKPDPDRQGIMDDENRMV |
| | | | NTPWYWLELARKGDMYTDNESSGERGLNKTAIAKIITQNK |
| | | | WSGGPAGLLARVTDLLVNPIDDVIPITLALYDAIRFGAGGIR |
| | | | AHGGYDMEPAVQFCRERLAT |
| YP_008409236 | Catdawg | 210 | MRIGGEYVGLGLGDASEEIGRIRDFMRRKFRSYAGNLPDT |
| | | | RNALGLPLFDEPMTVAVTEMQGRYLQSGELRDGLFIFGIIN |
| | | | AETKYVMGYLERPPGPDTRPLFITVCGTGVPWWVGPDAD |
| | | | TARACEDKLFWQPIGYPAKNFPMGKSITAGIDQCHVQFNR |
| | | | ADPGFMHRQRIERNGVVLGGYSQGAVVVSELWENNIKPA |
| | | | SGSLHWAKDYVVKAVTWGNPNREAGAVWPDYGGSPMA |
| | | | SLTSQGVSSTGMRDTPSWWRNYAHAGDLYAAAEPGDSQ |
| | | | QDKNAIWQIIRDLNFFTGTDSLLAQAIELSQMPVPRTIAAFK |
| | | | ALIDAGMFFAKGTGPHVDYGIGPAIEYLRS |
| YP_009638274 | Saintus | 211 | MSLDNHHIEELEQALRDVKRIGVQANPLLATSPAHVTGPT |
| | | | WQRTKDGKFYLPERTLGYQILYWMSQYLLIPGGPDAGQP |
| | | | FKPTREQGRFILWWYAVDEFGRFIYRNGLLRRLKGWGKD |
| | | | PLAGALALAELCGPVAFSHFDSDGNPIGKRKPAAWIQVAA |
| | | | VSQDQTRNTFSLFPVLASERLKEEFKLDFNKTIVYAKTIGG |
| | | | VIEAATSNPLTLEGKRPTFVIKNETQWWVETNNGHSMSEV |
| | | | IAGNVDKAAYGGCRSLAICNGHIPGQDSDAERDYDAHLDV |
| | | | LAGKAIDTGFLYDALEAPADTPLSEIPSPIEDPEGFEEGIAK |
| | | | LREGLIIARGDAVWLDLDTIIASMLDKRRPVTESRRKFLNQI |
| | | | NAHEDSWIAPHEWNACHPEEGVPPLAKGDRITLGFDGSK |
| | | | SNDHTALVACRVEDGCLFLIKTWDPEKYPGGEVPRDEVD |
| | | | ATVRSMKAGYDVVAFRADVKEFEAYVDQWGKDFRKTVQ |
| | | | VNASPGNPIAFDMRGNQKKFALDCERFLDAVLERELFHD |
| | | | DNPILRQHVFNARRHPTNYDAIAIRKASKDSSKKIDAAVCA |
| | | | VLAFGARQDFLMSKKNRTRRAAVIM |
| YP_009018053 | Thibault | 212 | MPLRLGDRNESVRQWRIKMNAWFGPLYTRLIGPLPMDTD |
| | | | EFGPRAKAWQEEYERRTNQVVDGEVSDNDLRALGVPVP |
| | | | TKVVIFTVAGTGARWDQGYPFDLGRWQDQSRVILQPIGY |
| | | | RAAVFPMGPSVNEGEVELIRQMHIHLDRDPSLNFILIGYSQ |
| | | | GAIVTSRVLRRMMSGDLAHYYDRCIAGVTFGNPMRERGH |
| | | | FVGSNDPGGQGLDPKPLVNTPTWWYDYAAKGDIYSSGP |
| | | | GNDDKQAAEHMTAIYQAVMGHFIIGNDSLTQQVIELFTNP |
| | | | FKEAPAVIKAIASGIGFVASNPPTAPHIEYHLRECVPGVTYF |
| | | | DHAMGYVRQVISANKRIA |
| YP_010013949 | MrMagoo | 213 | MAWIGWQEGMEGLPVLAAIGELRAKFSYGKQLELTQKFT |
| | | | PELTQAIKTFQVKKGGLRTDGVLDYATQAALGVPEALKPW |
| | | | VFTVAGTGAGWDAGYPADVARAVLDLFRWWGIVYPAKP |
| | | | FPMGDSVDVGIAELLKQMKLKLDRYPAAKFVLIGYSQGAIV |
| | | | TSMVWKRYIKGKDLEKRIIGSITFGNPCRELGVANGNVAE |
| | | | GIPIPEGRGIGDDRLQSTPEWWYDFAHGANSPFGRDIYTD |
| | | | TPDDDTGEMMTAVYRAVQKLKNLWSGVDSILEQVGEIIKR |
| | | | PIPEIYAAFRAVIYGGQFVTTQPWATYPHTNYIIDTAVAFLR |
| | | | RIAKN |
| NP_818354 | Omega | 214 | MSLWPEIRAMLNGEFYVPAGTIITANGTGVPDGAGPGFGA |
| | | | DLARELERVAEGMWIWKWINYPAAVMPMRPSINTLRLALK |
| | | | EMIRTTPGKIVLSAYSQSAVAFAYVWRDDILNPDGELHDR |
| | | | LDDIVAVILYGNPVRAPGIAYGNELGGQSAPGKLNGHVTG |
| | | | GIAGPDCLRPEECVHPVTGRRIVLDFANAGDLYAAAPVGA |
| | | | EPWVKETEVGHNETLIYEAVMDFNGRDILAFAKEIAQILTM |
| | | | PLSQVVPLVQAIINGLSFLVQGPKAPHWTYTIGPAVDYLVR |
| | | | TGNELRKN |
| AOZ64368 | Marcoliusprime | 215 | MATVDYRLGMSGDQVKVIKRLLIDGYYYVRERYPRMTAA |
| | | | SDVYDVYTQASVVEFQFRAGLPVTGVADYATQVRLGAVV |
| | | | PPPPPRQRIMVLTFSGTSADMWTGYPADVARALDPAIFY |
| | | | WQPVCYGPNGIPAIFPMGSSAKSGEAEGLRLLDEKAGDF |
| | | | DYVVLIGYSQGALPASRLMRRILSGDLQRFKSKLIAGVTFG |
| | | | NPMREKGHTFPGGADPGGHGLDPQCLVGTPDWWHDHA |
| | | | TKGDIYTCGSGGNDETANADMTFIYQLVQGDILGMLFGTG |
| | | | NPLDLLGALGGGLLGGLGGGLLGGKGGLQLPSGLVLPGV |
| | | | GLGQGGALTQHQRGLVEAVLALLTNPFAEVPPAVKAIVSG |
| | | | VGFIATNPPTAPHIEYHIREAAPGVTHLQHAIDYLRQVGAS |
| | | | VAARAA |
| YP_009950375 | Eponine | 216 | MPHPLNPTNKPVLLTLSGTGQNMWTGYAIDVAHRVEDVW |
| | | | FVQPVGYGPGGVPAVWPMGRSAKSGVDEGVRLVLQEHS |
| | | | DAPGYSISGYSQGGGAAAMLLDEFRHGRLTSFGDRLLGG |
| | | | VTFGNPFREKGSYAGAVDPGGRGIADTRTVDTPAGWAD |
| | | | YVDPGDIYANVPDNAVGDDMTLIYRLVWLDDVGDVLALVA |
| | | | KLMRLLVSPLREFPSVVEALVRGIMFYGSKPRCAAHVEYH |
| | | | LRECPGTGMTYVEHAARHLRQLGQQAAAA |
| NP_569765 | TM4 | 217 | MAWVGWQLGMQGEQVKVIQQKLIAKYQWVRDRYPRLTA |
| | | | SGVYDVNTQAAIVEFQFRAGLPVTGIADYATQVRLGAVAP |
| | | | APPPRQRIMVLTFSGTSADMWTGYPADVARALDPSIFYW |
| | | | QPVCYGPNGIPAIFPMGSSAKSGEVEGLRLLDEKARDFDY |
| | | | IVLIGYSQGALPASRLMRRILSGDLQRFKSKLIAGVTFGNP |
| | | | MREKGHTFPGGADPGGHGLDPQCLVNTPDWWHDYAAK |
| | | | GDIYTVGSGSNDEKANADMTFIYQLVQGDILGMMFGTGN |
| | | | PLDILGLLGGLGGGLLGGLGGGLLGGGKGGLQLPSGLVL |
| | | | PGVQGGALTDHQRGLVEAVLALLANPFAEVPAAVKAIVSG |
| | | | VGFIATNPPTAPHIEYHIREAAPGVTYFQHAIDYLRQVGAS |
| | | | VAARAA |
| YP_008410205 | Redno2 | 218 | MSLWPEIRAMLNGEFYVPAGTIITANGTGVPDGAGPGFGA |
| | | | DLARELERVAEGMWVWKWINYPAAVFPMRPSINALHLAL |
| | | | KEMIRATPGNLVLSAYSQSAVAFAYVWRDDILNPDGELHD |
| | | | RLDDIVAVILYGNPVRAPGIAYGNELGGQSAPGKLNGHVT |
| | | | GGIAGPDCLRPEECVHPVTGRRIVLDFANAGDLYAAAPVG |
| | | | AEPWVKETEVGHNETLIYEAVMDFNGRDIMAFAKEIAQILT |
| | | | MPLSQVIPLVQAIINGLTFLAQGPRAPHWTYSIAPAVDYLIR |
| | | | CGNELRKN |
| YP_010014099 | Reindeer | 219 | MWIGWQEGMSGLPVLAAKGELRAKFSYGKGLDLTEEFG |
| | | | PDLTEALKTFQRNKGGLRTDGVLDYATQKALGVLEALKP |
| | | | WVFTVAGTGAGWDAGYPADVARQVLDLYRWQGVSYPA |
| | | | QAFPMGRSVDAGVAELQRLLRERLDRYPAARFVLIGYSQ |
| | | | GAIVTSMVWEKFIKGSDLEGRIIGSITFGNPCRELNVANGN |
| | | | VAEGVSVPEGRGISDFRLRSTPSWWYDFAHGANSRFGR |
| | | | DIYTDTPDDDTGEMMTAIYRLVQDLKNIFVGTDSLVEQVG |
| | | | EMFRRPIPEVGAAMWAIFLGGQFVATRPYPTMPHITYDIN |
| | | | PAVAILRRIAA |
| AYQ98518 | Reptar3000 | 220 | MPHPLNPTNKPVLLTLAGTGQDMWTGYAIDVAHRVEDVW |
| | | | FVQPVNYGPGGVPAVWPMGASAKTGVDEGVRLVLQEHS |
| | | | DAPGYAISGYSQGGGAASMLLDEFRHGRLASFGDRLRG |
| | | | GVTFGNPFREKGSYAGTVDPGGRGIADTRIVDTPAGWAD |
| | | | YVDPDDIYANVPDNKVGDDMTLIYRLVWLDDAGDVLALVV |
| | | | KLLQLLTSPLREFPAVVEAIVRGIMFYGTKPPCAAHGEYHL |
| | | | RECPGTGMTHFEHAVQHLRQLGEQTAAA |
| YP_009605033 | Rey | 221 | MAWIGWQEGMEGLPVLAAIGELRAKFSYGKQLGLTKKFT |
| | | | PALTEAVKTFQRNKGGLRTDGVLDYETQKALGVLEALKP |
| | | | WLFTVAGTGAFWDAGYPADIARAVADLYRWQGVNYPAT |
| | | | AFPMGDSVDAGIAELVKLIKQRLDRYPAAKFVMIGYSQGAI |
| | | | VTSMVWKRYIKGTSLEERIIGSITYGNPCRELGVANGNVA |
| | | | EGIPVPEGRGIADDRLQSTPLWWYDFAHGANSPFGRDIY |
| | | | TDTPDDDAGEMMTAVYRAVQKLKNLWSGVDSILEQVGEII |
| | | | KRPIPEIYAAFRAIVYGGQFVTTQPWATYPHTNYTIDHAVAI |
| | | | LRRIAKN |
| AOQ28893 | Waterfoul | 222 | MSKPMLLTGQGTGADMFTGYPHDLGVRLRDEDRVELQPI |
| | | | GKYPASMFPMGPSVKIGVDVGVDLVLRAEARPAREVPDG |
| | | | YLVCGYSQGAWLVSDLLDEFRGGRLKHVAHKLAGGVTFG |
| | | | NPRRELDQSGGRGIADKLIVGTPDTWVDEFDPGDIYANVP |
| | | | NNDVGEDMTAIFKLVRLNGISDVVDLRSGLDLVGVLGGLL |
| | | | VPGVGQLGGLAGGLGALFGGAQEQNTIVEQVVEMLKSPL |
| | | | REFPAAVMAIVKAAVFFGRKPATAPHIEYHIRTVTPGGPTY |
| | | | FEHAVNHMRAMAA |
| APC43065 | Kerberos | 223 | MSKPWLFTVHGTGQPDPLGPGLPADTARDVLDIYRWQPI |
| | | | GNYPAAAFPMWPSVEKGVAELILQIELKLDADPYADFAMA |
| | | | GYSQGAIVVGQVLKHHILPPTGRLHRFLHRLKKVIFWGNP |
| | | | MRQKGFAHSDEWIHPVAAPDTLGILEDRLENLEQYGFEV |
| | | | RDYAHDGDMYASIKEDDLHEYEVAIGRIVMKASGFIGGRD |
| | | | SVVAQLIELGQRPITEGIALAGAIIDALTFFARSRMGDKWP |
| | | | HLYNRYPAVEFLRQI |
| YP_009012182 | Patience | 224 | MKGPDGSYIGIGLGDVSSEVLKIKQFINRKFSRFKLLETEIY |
| | | | DAAMEAAVRELQTIYKNNGTLTDPFIPGVVNLATKYAIGYL |
| | | | KKDVILPIHFSVEGHMSDMWIGPAAYVGEVLRAENRALHF |
| | | | PTGYDNRALPFNNKSGVEQLVQRVGAAEFFIDGKVIKFPP |
| | | | GTPWTASAFSQGAMIWCDFYRQYLMPGKPLHWRLKDLR |
| | | | AVICCGNPDREKGVCVDWIPDKPGPDRQGIMDDENRMV |
| | | | NTPWYWLELARKGDMYTDNESSGERGLNKTAIAKIITQNK |
| | | | WSGGPAGLLARVTDLLVNPIDDVIPITLALYDAIRFGAGGIR |
| | | | AHGGYDMEPAVQFCRERLAA |
| AZS06596 | JacoRen57 | 225 | MELKIGSNDQNTNGEVTPWQEWFKRYASSYAPPVDGYF |
| | | | GTPDEAAVRMLQARLGLVIDGVFGDRTAERVGYRWKNN |
| | | | QGGPVAQRRKIWIYTAPGSGANWDQGPSFQLGERCKNV |
| | | | LHINHQPLYYQKGGYLGFMGGDPKYSYNEVIWDQYLSLK |
| | | | YCMDTNPDWQDALAMRKQNPQAVVEVEGWFSGYSQSA |
| | | | DGMEEAVERLFGDGGPYELLRDRINGIIQFGNPSTRNTGI |
| | | | ARKVRPEWIYKLVTNLNYLNDFYAVAPDPIRPAFYKVIVEA |
| | | | EMELPFFVHVLQIAVPVILNIIPVFGGFFGPLGQLGVAAAA |
| | | | GIKDMNALGSLMGMAGTNKDQDVHQDLIEMLSLTGLIRNI |
| | | | PGLISVVSALPGLQAHGGYEFDPVMMNAAYDVVAAFRR |
| PKQ59713.1 | Mycobacterium | 226 | MAWVGWQLDMEGDQVKVIQTKLYAKYDWVKNRYPSLKA |
| | sp. MHSD3 | | ASGKYDKATQAAVVEFQFRTGLPVTGIADFATQLRLGSAV |
| | | | SAPPPKPKPRILLLTFSGTSADMWSGFPADLARALDPAIYY |
| | | | WQPVNYGPNGIPAVFPMGSSVKSGEVEGVRLLDEKADEY |
| | | | DFIVLVGYSQGAIVASRLKRRIQSGDLQRFKSKLIAGVTFG |
| | | | NPLREAGHTFPGGTDPGGYGLDPELLTNTEDWWHDYAA |
| | | | PGDIYTCASGTSDQRTNSDMTLIYELVQGDILKLIFGSDSS |
| | | | PLDILLTLSSGLLGGFKLPAAILLPGLGEGPAGALSTRQRG |
| | | | LVEAVIALFTNPFAEVPAMVKAIVSGIGFVAQSPPTAPHIEY |
| | | | HIREAVPGVTYLQHAINHLNFVGAGIKR |
| BBC67257.1 | Mycobacterium | 227 | MSYGLPTGTNINYGQPGFPDWVYQLGAAFNLRASTYPGH |
| | marinum | | QESDRVEAGYARNPNRQNRGIDWAGAVPDMDRFAEYLL |
| | | | STRGSLEQVIWQNPATGARIGVAGGKDVTQTAYYAADYS |
| | | | GHTDHVHTRQSEAIPMPDAPPKDTLFADVSEWQVPVDDS |
| | | | YPYPVLSIRVSDGSYQDRNFARNYTWMRAALNSGKLTFGI |
| | | | VYTYVRPQTWQSNAATVKQMIDAAGGLHPRVALMLDIES |
| | | | GGNPPGDQSGGINALYSALADYTGDPARIIGYGNVSDLNG |
| | | | MWRTKPPGIRLIVAGYGRLPTYPGMVAHQYTDGQGYGG |
| | | | GLPEGCPPFGNCDMNAANGLTPAEFAAACGISGDLQPEP |
| | | | DPEPGPPRPDPSPSDPPTTNSPCAGHASATKPSSKPSPR |
| | | | SATPYWAPTTGSEAGNAHLAHRHSRLSQRAALMVRHLAL |
| | | | VYRGTGGIIGEDYVSRVCQPLADLVQEENPPWAATMGGL |
| | | | PVAAAGAPSDPSMNKGAADALTASIPMIERAISENPQRRIII |
| | | | GGYSAGAYVAALVRRYVQQRHPDNYLCSFSLGDPTRPP |
| | | | GGAYYPFDASVQPGGQGIGSWHYGDVTDPRHCWLSNHT |
| | | | APPNLGPDMYAITPLGVTGEIMQAAYDMVTDFSFSDILTAT |
| | | | RAIVQAVPKIAEDMGIDVPDVLAALAGGIPGLAGYGIPLLV |
| | | | GALSGLIGFGNNDTLTGTAAGAAAARIGLTFLAAGTGPHIR |
| | | | YEVDEVWPGQTYLGLAIQHVRYWASTVQPDAA |
| WP_094360020.1 | Mycobacterium | 228 | MKIGGQWVGYGLGDTGETVAAMKDFLRRKFSYAAGLAP |
| | marinum | | DDIYDDAMVAVVVEMQARYGLPASGIMNYATQLRCGFVK |
| | | | PRQPTHERGTLFTCQGTGVDMWTGPPADTARAVTDLYF |
| | | | WQPVGNWPAAPFPMGPSIDAGRAELNVQIDAHPGDISMA |
| | | | GYSQGAMVVALTYMRDILPADGRLHHRLPDVKRVVTWG |
| | | | NPCRQQGKANGNKQAGWPIPEGHGVNDWLLTETPDSWL |
| | | | DYAHGANSPWGRDFYTDVQGDWGEDCTAICKFVMGQNI |
| | | | FAGPDSLLAQVIELVQRPIPETIAMFQAIINAGMFFAAQTGP |
| | | | HLNYDIGPAIAYLRS |
| WP_207548622.1 | Mycobacteroides | 229 | MSADGSKPVLLTASGTGADMWTGYPADVARRMEDLWYF |
| | chelonae | | QPINYRAAVFPMGASVDEGVNEGVRIVNEEIPVGTPTSLV |
| | | | GYSQGGSVVSRLLDEFRSGRLKHRQSDLVAGLTFGNPDR |
| | | | ELGSYAGTKDPGGRGISNSRIKNTPPWWCDLAEPGDIYT |
| | | | NVPNNDVGEDMTAIFRFVQLRGIDDLIGEDSLLEQFIEIITG |
| | | | DSAIGLPKLDNLLRIPGMLAKIGVTGPLSGFPAAVMAIIKGI |
| | | | AFFGAKPATAPHIEYHIREIEPGVTYFDWGVRYLRAAGEH |
| | | | ARARMSA |
| WP_064630528.1 | Mycobacteroides | 230 | MTGNEGSGRIWAYTVSGTWAAWNEGFPADVARALDPNA |
| | immunogenum | | FRWQPVAYPASFGPVPPASSPTLPSYAESVHEGVAELIRL |
| | | | INLNAGPFVLIGYSQGAEVTSRVLLEIQHGQLAHRQQDLL |
| | | | GGVTFGNPCRQSGHTWPGDTLSGHGIAALRIANTPSQW |
| | | | NDYAHGGDLYACVPDGQAGDNCTAVYQAVTQLQIHDPV |
| | | | QLIEEMLTAFSGKGGLGEQLWELLTNPFNLTSVFEAIVIAT |
| | | | RFATTQPPTLDHINYDADDVMDGSGRSSLRHAIDHLNALG |
| | | | RQAVAA |
| WP_076124670.1 | Mycobacterium | 231 | MSRPLFISVNGTGVPDPYGPGFSGDIGRALTDPWNNVMA |
| | sp. IS-836 | | SFWGPELANVFDWQPIGYDAAVMGMDKSARGAVYRKPG |
| | | | TYSSDDTGGIVAQVLARPKGTKHVLSGYSQGAIATGICLV |
| | | | ECYFDPKGPLNDRLSDLKGVVNFGDPKRSPGIANGNKVA |
| | | | GLPMPKKLDGYTTGGIAGPGCLKPDQTPDWLLSCALDGD |
| | | | LYAAAPVGDDPWHNEPLVGQIETRIYDFIQSGKLSTGIMAI |
| | | | AKGIAQEFEHPLANTVALVQAIVNGLTFAAQGVQAPHWLY |
| | | | GPFVPAMVEWILAQV |
| SKI64419.1 | Mycobacteroides | 232 | MAWRGYELGMTDPKDGDGNIVPGGMIWQIQDKLKRKYA |
| | abscessus subsp. | | SYTGAVVVSGRYDGATFAAVGEFQNRAGLIGTGVKPDEV |
| | abscessus | | GIANYATLVRMGVVTTTPPRAPLTIFTAAGTWSDMWTGLQ |
| | | | ADVARALDRRYFFWQPIWYPASFGPVGGGPAPSYEESVA |
| | | | LGVEEGIRLIKATPGQFALCGYSQGAEVVSRILIELVSGRL |
| | | | KDRLKDCLWFVAFGNPARQPGVCVGRDPGGSGISGIRLL |
| | | | VPESVTVLDYAIDGDMYCTTPDGTEGGTNMRAVYKALTK |
| | | | MQIHDPGRDIISALTGDPSLMRQLIKLFSDPVKGGIGLVDA |
| | | | LFRLAKFAITGAHGRYGQYEVFPGVTPVRHAIETLNADAA |
| | | | RLLAA |
| WP_079634228.1 | Mycobacteroides | 233 | MIVSGQFVGYGRGDTGPEVERVRYYLTEKFAWARAMGIT |
| | abscessus | | HGDVFDELTEKVLIRFQIIVGLPATGIANYATRLRLYGLKPT |
| | | | PWQRITVYTFAGTWSASDWGIQADVARGLDASFFTWVPV |
| | | | EYPASFGPIPGGPSAGGGAPSYQESVAAAVSTGIRQVNN |
| | | | TPGKLMIGGYSQGGEAASLLLKEILGGSLTHRRGDLVGGY |
| | | | TFGNPSREANHTWPGDSLPGRGISPNRIVGTPSTWHDYA |
| | | | HAGDMYTATPNDDAGDDITAVYSVLTRLQIHDPWELAQSI |
| | | | VAALKGKGGLVEQITELLGNPLHLIDAARAAGIALQFLASG |
| | | | LRDHGNYWEDDVMDGSGRTSVQHAIDSMNAYGRKALAA |
| TMS47311.1 | Mycobacterium | 234 | MRRRHRDHGALPLERQQGRDARPATPGLISAGRNSSTAL |
| | sp. DBP42 | | LVLVVGPTASIGFVTPARADTPMIRLGFRGRSVLMAWRGF |
| | | | ELTDPPMSGPDIALIRGKLVAKFEWARTMGVTEGDVYDRI |
| | | | TADAVAEFQRRVGLPENGIADFKTRVRLGSWPPPPPPRH |
| | | | AALTFRGTGGIVGVDYTSRVAHAAGLEEIPILYPGSMGGIP |
| | | | VGAESNPNAPSGNDSVEVAVELAVDWIERHPSRTFCLLG |
| | | | YSQGAIAASKVRAELLPGGRLERFAGNYFCGMMVANPSR |
| | | | AFGHTFFLGPVPDGEGISNFHVPREACTWDWCELVQPDD |
| | | | FYANVPLGDVGDVCRQGQNIVMDTTVSDPIGMMQKVIPH |
| | | | LIKMLDEAGVDLPPSPLGILNGVWAGLVSSLLPGVVPAQF |
| | | | GAETAAAVHAARLALTFFAAQPPTKPHITYEFVEVLPGRTY |
| | | | LQLGIDHVRDWSGRTPVRT |
| WP_211697615.1 | Mycobacterium | 235 | MGFKMGSSGPEVGRWQEFMRRKFASYSEELPVDEHYG |
| | spongiae | | YFDGVVVNEAKRRLGLPQDGVADDDFLIRIGFSAGQARPL |
| | | | STTWVYSAAGTRAPWSIGPPFEIGEHAKRRGLRHQPVDY |
| | | | PAGGFFGPPDPTMSFNESIKVLRNEWARLLRLNTAGDIVTI |
| | | | SYSQGADGMQRATAELFGDGGEFASQRHRLRRAIMVGN |
| | | | PTRPSGPTKIGNDPRGAGIARWHPPDWLQAITFDIVTHYD |
| | | | MYACAEDTTLVPLFYPWFTRAETELSFVAYSAQVIVPTVA |
| | | | SFYNIFIPPILGGLFGPLGVQALALVTRIPVDTLNELFKGISG |
| | | | EDPDPELVEALSAKGLLSDIPGLIKSLVALGGIRTHNEYHLP |
| | | | RPEFGNRTGIDVGIGLIDEIL |
| WP_236054790.1 | Mycolicibacterium | 236 | MVQEQPIRTEKSAADPMPRHAVLTFAGTFEAPGTGFPSD |
| | sp. SM1 | | WVKQANPDLVYEVPVVAPYSFGPIPPDSPTAPSYAESVEIA |
| | | | VTYATTWITAHPRQTFAIGGYSQGAEAASRVLASIMGGPL |
| | | | QWARPNLIGGYTFGNPARGLGHTFPGGTDPGGRGISSFN |
| | | | LTTAQIPKNTRGIDTWWDFANPGDLYTTTPDDQAGADLTA |
| | | | VYDEAVQLSIQRNLILNLIAGLTERGGLLQQAIGLVTNPLAG |
| | | | GPALAEAITLAVRFYDTSPPGLAHLDYNTVDALPGQSSVA |
| | | | VAVNHLNSIAAATPTHSAA |
| ORV92819.1 | Mycobacterium | 237 | MTGWADLAANIAGTKLVRHAVLTYNGTWGAGLVQYPSDV |
| | interjectum | | VNGLAQYVDDGLCEEVPCPYPASFGPIGGNAAAPSYQQS |
| | | | IQDALGWSADWFDANPNRTFVLDGYSQGAEAASRVYLEL |
| | | | VAGTALEENFVGGITFGNPCKMAGAAAPGVSSLGPQWR |
| | | | GISSVNMTSLPAINGQVVWADYAHNTANGDAGNDMYPQ |
| | | | VPNTAVGTIMTDVYTTATQAQLNNPTAFLQSMVTDLMQLV |
| | | | EDSGLLTGLKGGAAGLLAMGAGAAIGFLVDLIGGVNINAT |
| | | | GAQADVAAAVLGLQFLAAPGGPTGPHISYLGELPGYSNLV |
| | | | ADAVNFLHTIATLTPARA |
| WP_231984054.1 | Mycobacterium | 238 | MLAGAIAVAGSALVTAGTAAAQPACPDLHWIGAAGSGER |
| | sp. 852013- | | GAEVTMNDGMGRVVFNSMQDLHQLVQRDGRTMTAEAV |
| | 51886_SCH5428379 | | NYPATPVPADDSILGWAGFINSVDAGTAALNAQYAAFVQR |
| | | | CPSSKVVLAGYSQGAMVVHRNLQAFSQSPNLAAALLVAD |
| | | | GDRLPTDATYNLGSVTSVPGAGKGVAQDWPILAHAPAPL |
| | | | PLDVAARTVSVCDLGDAVCDYDPEAEDAMNPTAVAIHTS |
| | | | YARSAAGGYRWTAPLYQLIGAAPTANQVSAAAG |
| ATW60721.1 | Bjanes7 | 239 | MIELLFVDGTWSRPGARSPVGEALRKALDPSKVKFTYVDY |
| | | | PADFGPATGVGDVSYADSVMAGVAALSLAVERSQFDVVV |
| | | | AGYSQGAAVAVHYALRVLPRKPKHIVLALATVGDPHQPVH |
| | | | NGRSGIAGAITLSLRSFRRFVPGDPIADLDLGSPVRSAADL |
| | | | SRWMSVRSPEAARAWAYRTAAELPTRAQRWWEPWNW |
| | | | AAIGRAGEAIRNYLGTAHSTDYISQGHVRRLARDIESVA |
| QLF838341 | Moosehead | 240 | MIELLWVDGTWAPRGGSPASEALRRALDPRKVKFTYVNY |
| | | | PADFGPATGMGDLSYEESKSIGAAALDRAVEASPHLVVV |
| | | | GGYSQGAGVAVKYARDILPKRPRHQVLAVATLGDPHTPV |
| | | | HHGRSGIAGALHVPRPRFTEWASGDPIADLPLGSPLRIVA |
| | | | DLTGWMSVRTPEAARAWAFKTAERLGRAQPWWNPFRW |
| | | | HDFARAGEDIRNYLGTAHSTDYDSGGHARRLARRIESVA |
| YP_010013529.1 | Kumao | 241 | MAWDGWKEGMAGPPVLAAKRELKRKFSYAKHLVENTFF |
| | | | DHDLTVALMTYQVAKNIELAKRGEPLLRTDGVLDWYTQKV |
| | | | LGLLDRKVVIFTVSGTGAVWSQGYPFDVAMRQDQSKVIV |
| | | | QPIGYPAAVFPMEHSANEGERELVAQMRRHLDANPSYVF |
| | | | ILIGYSQGAMVVSRVLRRMMSGDLRQYFDRCIAGVTFGN |
| | | | PLRERGHFTGASDPGGQGLDPECLVDTPSWWHDYAIPG |
| | | | DIYTCGPGNYDLAALEHMRAIYLAVQGHELTGRDNLGEQV |
| | | | LEVLMNPFAEVPAVVKAIVSGLGFVTANPPTAPHIEYHIRE |
| | | | CFPGVTHFEHAVDYVRRAVSAGMRIE |

Additional sequences were identified that may be useful in the compositions and methods described herein. In some embodiments, the composition includes an isoamylase that includes an amino acid sequence having at least 85% (e.g., at least 90%, 95%, 97%, 99%, or 100%) sequence identity to of any one of SEQ ID NOs: 242-392 as shown in Table 4 below.

TABLE 4
Isoamylase Sequences

| | | SEQ | |
|---|---|---|---|
| | | ID | |
| NCBI ID | Species | NO: | Sequence |
| ARBA000 | Candida glabrata | 242 | MSAHRTLLLRLSDSGEPVTSCSYGQGVLTLPSLPLPQGKK |
| 20723 | (strain ATCC | | LGDMPVYTVKLAIPAGSPVTRDGLIWTNCPPDFSTQFDRE |
| | 2001/CBS 138/ | | KFYKKIIKTSFHEDDHIDLDIYVPGTYCFYLSFKNDKDELET |
| | JCM 3761/ | | TRKFYFVVLPILSVNDKFIPLNSIAMQSVVSKWMGPTIKDW |
| | NBRC 0622/ | | EKVFARVASKKYNMIHFTPLQHRGESNSPYSIYDQLEFDP |
| | NRRL Y-65) | | TVFKSEKEVADMVERLRTEHNILSLTDIVFNHTANNSQWL |
| | | | LDHPEAGYNHKTSPHLISAIELDKKLLDFSEQMEALGYPVD |
| | | | LKTVDDLIKVMDGIKEHVIGELKLWEFYVVDVKQTVSELRE |
| | | | KWGNSKSWSDDNIPSKDDSTNLAQFVRDNATEPGFGSL |
| | | | GERGSNKINIDKFAAILKKLHSEDYNNGIEELATKILNDINLP |
| | | | FYKEYDDDINEVLEQLFNRIKYLRIDDHGPKQGPITKKLPLS |
| | | | EPYFTRFKAKDGEEYALANNGWIWDGNPLVDFASSQSKA |
| | | | YLRREVIVWGDCVKLRYGKGPSDSPYLWERMSKYVEMN |
| | | | ARIFNGFRIDNCHSTPLHVGQYFLDVARRVNPNLYVVAEL |
| | | | FSGSEAMDCLFVERLGISSLIREAMQAWSEEELSRLVHRH |
| | | | GGRPIGSYKFVPLDDFPYPADVKIDEEYCAYNPDDHSVKC |
| | | | VSEIMIPKTLTATPPHALFMDCTHDNETPNQKRTVEDTLP |
| | | | NAALVAFCSSAIGSVYGYDEVFPQLLDLVQEKRTYSCAEN |
| | | | TGISKVKTLLNNMREEIASEAVDIEDSEMHVHHDGQYITFH |
| | | | RTNAKNGKGWYLVARTKFHSSGDQMLPRIKLSQTKATFK |
| | | | AAFSLERTGDAPISDEIIEGIPTKLRELTGFDIGFDENTKET |
| | | | SILLPQDFPQGSIVIFETQQLGIDDSLDHFIRSGAIKATEKLS |
| | | | LESINYVLYRAEQEEYDYSEGRSGAYDIPDYGKPVYCGLQ |
| | | | GWVSILRKIIFYNDLAHPLSNNLRNGHWAVDYVVNRLDLY |
| | | | KDKEGVAEVQEWLRSRMERIKQLPSYLVPSFFALVVGIMY |
| | | | GCCRLRAMQLMSDNVGKSTVFVQSLAMTSIQMVSAMKS |
| | | | TSILPDQNIAAMAAGLPHFSTNYMRCWGRDVFISLRGLLL |
| | | | TTGRYEEAKEHILAFAKTLKHGLIPNLLDAGRNPRYNARDA |
| | | | AWFFVQAIQDYVTIVPGGVSLLQEKVTRRFPLDDEYIPYD |
| | | | DPKAFSYSSTIEEIIYEILNRHAGGIKYREANAGPNLDRVMK |
| | | | DEGFNVEVNVDWETGLIHGGSQFNCGTWMDKMGESEKA |
| | | | NSVGVPGTPRDGAAVEINGLLKSCLRFVLQLSKDGKFKYT |
| | | | EVTKPDGSKISLSSWNDLLQENFERCFYVPKNKEDDNKF |
| | | | EIDATIINRRGIYKDLYRSGKPYEDYQFRPNFTIAMVVAPEL |
| | | | FTPDYAAGAIELADQVLRGPVGMRTLDPSDYNYRPYYNN |
| | | | GEDSDDFATSKGRNYHQGPEWVWCYGYFIRAYHYFNFL |
| | | | TNPKCQVEGSAKKLKPSSYLYRKLYSRLLKHREWIENSP |
| | | | WAGLAELTNKDGEVCNDSSPTQAWSTGCLLDLFYDLWIS |
| | | | YEE |
| ARBA000 | Candida glabrata | 243 | MSAHRTLLLRLSDSGEPVTSCSYGQGVLTLPSLPLPQGKK |
| 20723 | (strain ATCC | | LGDMPVYTVKLAIPAGSPVTRDGLIWTNCPPDFSTQFDRE |
| (10X-His) | 2001/CBS 138/ | | KFYKKIIKTSFHEDDHIDLDIYVPGTYCFYLSFKNDKDELET |
| | JCM 3761/ | | TRKFYFVVLPILSVNDKFIPLNSIAMQSVVSKWMGPTIKDW |
| | NBRC 0622/ | | EKVFARVASKKYNMIHFTPLQHRGESNSPYSIYDQLEFDP |
| | NRRL Y-65) | | TVFKSEKEVADMVERLRTEHNILSLTDIVFNHTANNSQWL |
| | | | LDHPEAGYNHKTSPHLISAIELDKKLLDFSEQMEALGYPVD |
| | | | LKTVDDLIKVMDGIKEHVIGELKLWEFYVVDVKQTVSELRE |
| | | | KWGNSKSWSDDNIPSKDDSTNLAQFVRDNATEPGFGSL |
| | | | GERGSNKINIDKFAAILKKLHSEDYNNGIEELATKILNDINLP |
| | | | FYKEYDDDINEVLEQLFNRIKYLRIDDHGPKQGPITKKLPLS |
| | | | EPYFTRFKAKDGEEYALANNGWIWDGNPLVDFASSQSKA |
| | | | YLRREVIVWGDCVKLRYGKGPSDSPYLWERMSKYVEMN |
| | | | ARIFNGFRIDNCHSTPLHVGQYFLDVARRVNPNLYVVAEL |
| | | | FSGSEAMDCLFVERLGISSLIREAMQAWSEEELSRLVHRH |
| | | | GGRPIGSYKFVPLDDFPYPADVKIDEEYCAYNPDDHSVKC |
| | | | VSEIMIPKTLTATPPHALFMDCTHDNETPNQKRTVEDTLP |
| | | | NAALVAFCSSAIGSVYGYDEVFPQLLDLVQEKRTYSCAEN |
| | | | TGISKVKTLLNNMREEIASEAVDIEDSEMHVHHDGQYITFH |
| | | | RTNAKNGKGWYLVARTKFHSSGDQMLPRIKLSQTKATFK |
| | | | AAFSLERTGDAPISDEIIEGIPTKLRELTGFDIGFDENTKET |
| | | | SILLPQDFPQGSIVIFETQQLGIDDSLDHFIRSGAIKATEKLS |
| | | | LESINYVLYRAEQEEYDYSEGRSGAYDIPDYGKPVYCGLQ |
| | | | GWVSILRKIIFYNDLAHPLSNNLRNGHWAVDYVVNRLDLY |
| | | | KDKEGVAEVQEWLRSRMERIKQLPSYLVPSFFALVVGIMY |
| | | | GCCRLRAMQLMSDNVGKSTVFVQSLAMTSIQMVSAMKS |
| | | | TSILPDQNIAAMAAGLPHFSTNYMRCWGRDVFISLRGLLL |
| | | | TTGRYEEAKEHILAFAKTLKHGLIPNLLDAGRNPRYNARDA |
| | | | AWFFVQAIQDYVTIVPGGVSLLQEKVTRRFPLDDEYIPYD |
| | | | DPKAFSYSSTIEEIIYEILNRHAGGIKYREANAGPNLDRVMK |
| | | | DEGFNVEVNVDWETGLIHGGSQFNCGTWMDKMGESEKA |
| | | | NSVGVPGTPRDGAAVEINGLLKSCLRFVLQLSKDGKFKYT |
| | | | EVTKPDGSKISLSSWNDLLQENFERCFYVPKNKEDDNKF |
| | | | EIDATIINRRGIYKDLYRSGKPYEDYQFRPNFTIAMVVAPEL |
| | | | FTPDYAAGAIELADQVLRGPVGMRTLDPSDYNYRPYYNN |
| | | | GEDSDDFATSKGRNYHQGPEWVWCYGYFIRAYHYFNFL |
| | | | TNPKCQVEGSAKKLKPSSYLYRKLYSRLLKHREWIENSP |
| | | | WAGLAELTNKDGEVCNDSSPTQAWSTGCLLDLFYDLWIS |
| | | | YEEHHHHHHHHHH |
| QJW96644.1 | Frigoriglobus | 244 | MPPFRTSRGRPLPLGPSLTPDGTNFALLCRHGRTVTLVIL |
| | tundricola | | PAEGGSTPLAELPLDARTNRTGDHWHIRVHDLPEAFCYG |
| | | | WRVDGPHGPRTRFDPSRLLLDPSAVILSHGAEWAGTCET |
| | | | DPQRTSRRSMYRRGTRYNWDDDCPPLVDYEDTVIYEVH |
| | | | VRGFTCHPSSGVAAPGTFRGLVEKIPYLKWLGVTAVELM |
| | | | PVFEWDECDCPFVNPATGEKLTNFWGYNPIAFAAPKAAF |
| | | | AASAKQLGQTNEFRDMVKAMHAAGIEVILDVVFNHTGEG |
| | | | DDRGRTFSFRGLDNELYYLLDDSGRYLNFSGCGNTVNCN |
| | | | HPVVRDLIMTCLRYWVEDMHVDGFRFDLASILGRDRRGN |
| | | | VMVEPPVIESITEDGVMADTKLIAEPWDAGGLYQVGQFPF |
| | | | GRRWSEWNGQYRDDVRRFWKGDPGLTGAMASRVCGS |
| | | | SDLYQWNGRLPRHSVNFVTAHDGFTLYDLVSYNEKHNEA |
| | | | NGEGNRDGSNDNHSWNCGAEGPTTDPAVLALRRRQAR |
| | | | NLMTTLMISQGVPMLLAGDEFLRTQQGNNNAWCQDNDV |
| | | | SWVNWTLAEGNKDFVRFVRELIHLRKRHPALRRRRFFAG |
| | | | EFRSGDAPRPAPARAAEPHVIDVFPPAGPVRPGDAGLHP |
| | | | ASDAATGLARSGRGADAPVPALADIHWHGVEPFRPDWG |
| | | | YNARVLAFALDGRFTGREGDPDYQIDEDFYVVFNAWSEA |
| | | | LRFRIPASPTRRRWRRLIDTALPAPGDFVAEGEGPVVADG |
| | | | GTYTVAGFSTLVLISEP |
| QJW96644.1 | Frigoriglobus | 245 | MPPFRTSRGRPLPLGPSLTPDGTNFALLCRHGRTVTLVIL |
| (6X-His) | tundricola | | PAEGGSTPLAELPLDARTNRTGDHWHIRVHDLPEAFCYG |
| | | | WRVDGPHGPRTRFDPSRLLLDPSAVILSHGAEWAGTCET |
| | | | DPQRTSRRSMYRRGTRYNWDDDCPPLVDYEDTVIYEVH |
| | | | VRGFTCHPSSGVAAPGTFRGLVEKIPYLKWLGVTAVELM |
| | | | PVFEWDECDCPFVNPATGEKLTNFWGYNPIAFAAPKAAF |
| | | | AASAKQLGQTNEFRDMVKAMHAAGIEVILDVVFNHTGEG |
| | | | DDRGRTFSFRGLDNELYYLLDDSGRYLNFSGCGNTVNCN |
| | | | HPVVRDLIMTCLRYWVEDMHVDGFRFDLASILGRDRRGN |
| | | | VMVEPPVIESITEDGVMADTKLIAEPWDAGGLYQVGQFPF |
| | | | GRRWSEWNGQYRDDVRRFWKGDPGLTGAMASRVCGS |
| | | | SDLYQWNGRLPRHSVNFVTAHDGFTLYDLVSYNEKHNEA |
| | | | NGEGNRDGSNDNHSWNCGAEGPTTDPAVLALRRRQAR |
| | | | NLMTTLMISQGVPMLLAGDEFLRTQQGNNNAWCQDNDV |
| | | | SWVNWTLAEGNKDFVRFVRELIHLRKRHPALRRRRFFAG |
| | | | EFRSGDAPRPAPARAAEPHVIDVFPPAGPVRPGDAGLHP |
| | | | ASDAATGLARSGRGADAPVPALADIHWHGVEPFRPDWG |
| | | | YNARVLAFALDGRFTGREGDPDYQIDEDFYVVFNAWSEA |
| | | | LRFRIPASPTRRRWRRLIDTALPAPGDFVAEGEGPVVADG |
| | | | GTYTVAGFSTLVLISEPHHHHHH |
| VEG28189.1 | Actinomyces | 246 | MPAPFAIDESTWAEATWPLGTHLTAEGLTVAVHAPAATRV |
| | howellii | | QLEVYPEALGAGAQAVFLPSRGADGAWRARLGGLGAGA |
| | | | LLGFRVWGPNWPYEEGWVPGSEAGFIADLDSEGNRFNP |
| | | | NKVLFDPYSREVSHTVLTDRLAELGVDDGVFGTGSDLVG |
| | | | GVPRRLIDTAPYAPKGVVVAEDDHVPARASAPRIPAEQAII |
| | | | YEAGVRQLTGHPSVARLSDLLAGEPGFEDWVDIPPLYQGT |
| | | | YKGAGMLAPYLKAAGVTTVELLPVHETNASESGRPGATN |
| | | | AWGYMTLAFFAPNRRYAADTSWGGPTREFKEMVAAFHA |
| | | | AGLEVYLDWVYNHTAEGGNWNGDVATTGFTSLGGFATAE |
| | | | YYQMTGSKMLVDGATGTSNQMSYSSPAARQLVLDSLRY |
| | | | WFSQMGVDGFRFDLATVLGRLPAQSAPDDWGGLKRFFN |
| | | | EHPLLTEIAALAQAEGIEVIAEAWDLWGYEVGNFPRGWGE |
| | | | WNGRYRDAVRRFTKGDGNTGEFIDMVNGDYHHFEDNGG |
| | | | PQKSINFVSAHDGFTMADLVSYQTKVNDQPHPFGPSDGG |
| | | | SDDNMSWDSGGDQGLRRQRLRNLWVILMVSRGVPMLVA |
| | | | GDEMGRTQNGNNNPWALDTVAMRTNYDMVATNAPQQV |
| | | | PVGDPGGAYHDNLGVFGSRDERVNPLFRLATFLMRLRHR |
| | | | HRALHEAAWGDALAGGTDVSYLFRTPSGQGVLGEGDRA |
| | | | LAIHVDAPGEGLWVMVNMAAEPVDFAPPPPGRGPTGDS |
| | | | GRDQGAGTTVWRRLVDTGVWAEPEGNFWPEGTGEVLS |
| | | | GTVTVGPWSVVVCQAVSAAQG |
| VEG28189.1 | Actinomyces | 247 | MPAPFAIDESTWAEATWPLGTHLTAEGLTVAVHAPAATRV |
| (6X-His) | howellii | | QLEVYPEALGAGAQAVFLPSRGADGAWRARLGGLGAGA |
| | | | LLGFRVWGPNWPYEEGWVPGSEAGFIADLDSEGNRFNP |
| | | | NKVLFDPYSREVSHTVLTDRLAELGVDDGVFGTGSDLVG |
| | | | GVPRRLIDTAPYAPKGVVVAEDDHVPARASAPRIPAEQAII |
| | | | YEAGVRQLTGHPSVARLSDLLAGEPGFEDVVDIPPLYQGT |
| | | | YKGAGMLAPYLKAAGVTTVELLPVHETNASESGRPGATN |
| | | | AWGYMTLAFFAPNRRYAADTSWGGPTREFKEMVAAFHA |
| | | | AGLEVYLDVVYNHTAEGGNWNGDVATTGFTSLGGFATAE |
| | | | YYQMTGSKMLVDGATGTSNQMSYSSPAARQLVLDSLRY |
| | | | WFSQMGVDGFRFDLATVLGRLPAQSAPDDWGGLKRFFN |
| | | | EHPLLTEIAALAQAEGIEVIAEAWDLWGYEVGNFPRGWGE |
| | | | WNGRYRDAVRRFTKGDGNTGEFIDMVNGDYHHFEDNGG |
| | | | PQKSINFVSAHDGFTMADLVSYQTKVNDQPHPFGPSDGG |
| | | | SDDNMSWDSGGDQGLRRQRLRNLWVILMVSRGVPMLVA |
| | | | GDEMGRTQNGNNNPWALDTVAMRTNYDMVATNAPQQV |
| | | | PVGDPGGAYHDNLGVFGSRDERVNPLFRLATFLMRLRHR |
| | | | HRALHEAAWGDALAGGTDVSYLFRTPSGQGVLGEGDRA |
| | | | LAIHVDAPGEGLWVMVNMAAEPVDFAPPPPGRGPTGDS |
| | | | GRDQGAGTTVWRRLVDTGVWAEPEGNFWPEGTGEVLS |
| | | | GTVTVGPWSVVVCQAVSAAQGHHHHHH |
| ABY22167.1 | Renibacterium | 248 | MTDIVTATVLGAAASRPFPLGLSAPLQGDPSDVVNVAVW |
| | salmoninarum | | APDFAELVLYFAAPGQPWRAMTLPEFSDGVHHGVVEGM |
| | (strain ATCC | | PIGSRYGFAAGDVEPEATQLLLDPYARTIDSSARLLGVRM |
| | 33209/DSM | | AASFDWGNDVSPRTPWRDSVIYEAHIKGLTQLHPDIPAEI |
| | 20767/JCM | | RGSYAGLAHPAMIRHLRGLGVSAVELLPIHAHADEQHLRD |
| | 11484/NBRC | | LGLPNYWGYNTIGYFAPQASYASAAAQAAGPQAVQDEFK |
| | 15589/NCIMB | | GMVKLLHAAGIEVILDVVYNHTAEGKHDEPALSWRGLAEN |
| | 2235) | | RYYRFDCDPSSGAEYLDTTGCGNTLDFSEPKVIQMALDSL |
| | | | RYWVEEFHIDGFRFDLAVTLVRDGANSFSPQHPFLLAATT |
| | | | SQALAGVKLISEPWDVGYDGWHTGQFPVGWADWNDHF |
| | | | RDSVRDFWIADQGSIANGGSGGGLARLADALSGSTRLFA |
| | | | ASGRSQLASINLVTAHDGFTLADLSAYNGKHNEANGEEN |
| | | | RDGSDNNRSWNHGVEGPSEQSDIRAARAQTSRNVMATL |
| | | | LLSLGVPMITAGDEMGRSQGGNNNAYCQDNEISWVDWS |
| | | | AVDREMLSATRRMIRLLKDYLSAQPSTYPARDESSFLHWF |
| | | | DVTGQPLGPEQWQDGSQRVLQMLLGSPDGLTDGLVVFN |
| | | | ATADEVSVTLPEAEGRSFELRFSTSANYAKQQGTVITGGS |
| | | | QLAHSANTITIYKA |
| ABY22167.1 | Renibacterium | 249 | MTDIVTATVLGAAASRPFPLGLSAPLQGDPSDVVNVAVW |
| (6X-His) | salmoninarum | | APDFAELVLYFAAPGQPWRAMTLPEFSDGVHHGVVEGM |
| | (strain ATCC | | PIGSRYGFAAGDVEPEATQLLLDPYARTIDSSARLLGVRM |
| | 33209/DSM | | AASFDWGNDVSPRTPWRDSVIYEAHIKGLTQLHPDIPAEI |
| | 20767/JCM | | RGSYAGLAHPAMIRHLRGLGVSAVELLPIHAHADEQHLRD |
| | 11484/NBRC | | LGLPNYWGYNTIGYFAPQASYASAAAQAAGPQAVQDEFK |
| | 15589/NCIMB | | GMVKLLHAAGIEVILDVVYNHTAEGKHDEPALSWRGLAEN |
| | 2235) | | RYYRFDCDPSSGAEYLDTTGCGNTLDFSEPKVIQMALDSL |
| | | | RYWVEEFHIDGFRFDLAVTLVRDGANSFSPQHPFLLAATT |
| | | | SQALAGVKLISEPWDVGYDGWHTGQFPVGWADWNDHF |
| | | | RDSVRDFWIADQGSIANGGSGGGLARLADALSGSTRLFA |
| | | | ASGRSQLASINLVTAHDGFTLADLSAYNGKHNEANGEEN |
| | | | RDGSDNNRSWNHGVEGPSEQSDIRAARAQTSRNVMATL |
| | | | LLSLGVPMITAGDEMGRSQGGNNNAYCQDNEISWVDWS |
| | | | AVDREMLSATRRMIRLLKDYLSAQPSTYPARDESSFLHWF |
| | | | DVTGQPLGPEQWQDGSQRVLQMLLGSPDGLTDGLVVFN |
| | | | ATADEVSVTLPEAEGRSFELRFSTSANYAKQQGTVITGGS |
| | | | QLAHSANTITIYKAHHHHHH |
| AEE17612.1 | Treponema | 250 | MDSIKVHPGRPMPYGATPVRDGINFSVFSRNGTSVILDIFK |
| | brennaborense | | KPEDSEPYFSYEFDPVVNRTGDMWHVRLEGVETGALYLY |
| | (strain DSM | | RVDGPFAPENGHRFNKNHYLLDPYAKALTDMSIFANLPKD |
| | 12168/CIP | | YAAPIDKLDVEFGKRRSARHFPKCIVIDDADFDWQGDQPL |
| | 105900/DD5/3) | | NYKLKNCVLYETHLKGFTASPTSAVAHPGTYRGMTEKIPY |
| | | | LKSLGITSVELMPIQEFDEFENANTNPRTGKRLKNHWGYS |
| | | | TISFFAPKTSYAADRTPGGAVREFKEMVREMHKNGLEVIL |
| | | | DIVFNHTAEGNEHGLTLNFRGFDNSIYYILEDKHKQYYKNF |
| | | | SGCGNTVNCNHPVVRSFIIDCLRYWVIEMHVDGFRFDLAS |
| | | | ILGRDRNGNLIKEPPVLERIAEDPILGRTKIIAEAWDAGGAY |
| | | | QVGTFPGGRWAEWNDRFRDEIRRFWRGDDFLCTAAATR |
| | | | MTGSADLYQDDGRKPYHSVNFITSHDGFTLNDLVSYNGK |
| | | | HNEENGEHNRDGSDNNSSYNYGYEGPTANKAIEGIRTRQ |
| | | | VKNMLLTLLLSQGTPMLLSGDEFRRTQGGNNNAYCQDNE |
| | | | LSWLNWTNQETYAEIVTFLRKAIHTRLTHPVFRRPDFFEG |
| | | | QDHSANLLPDINWFASDGKTPDWSQLNHFLAFRLDGSKA |
| | | | EIFADRDDNDFFIMQNTGATDMTVKTPSLNKGKKWYRFID |
| | | | TSVPAPNDFTDHGSEELLEEQKTYILPARTMAVLLGK |
| AEE17612.1 | Treponema | 251 | MDSIKVHPGRPMPYGATPVRDGINFSVFSRNGTSVILDIFK |
| (6X-His) | brennaborense | | KPEDSEPYFSYEFDPVVNRTGDMWHVRLEGVETGALYLY |
| | (strain DSM | | RVDGPFAPENGHRFNKNHYLLDPYAKALTDMSIFANLPKD |
| | 12168/CIP | | YAAPIDKLDVEFGKRRSARHFPKCIVIDDADFDWQGDQPL |
| | 105900/DD5/3) | | NYKLKNCVLYETHLKGFTASPTSAVAHPGTYRGMTEKIPY |
| | | | LKSLGITSVELMPIQEFDEFENANTNPRTGKRLKNHWGYS |
| | | | TISFFAPKTSYAADRTPGGAVREFKEMVREMHKNGLEVIL |
| | | | DIVFNHTAEGNEHGLTLNFRGFDNSIYYILEDKHKQYYKNF |
| | | | SGCGNTVNCNHPVVRSFIIDCLRYWVIEMHVDGFRFDLAS |
| | | | ILGRDRNGNLIKEPPVLERIAEDPILGRTKIIAEAWDAGGAY |
| | | | QVGTFPGGRWAEWNDRFRDEIRRFWRGDDFLCTAAATR |
| | | | MTGSADLYQDDGRKPYHSVNFITSHDGFTLNDLVSYNGK |
| | | | HNEENGEHNRDGSDNNSSYNYGYEGPTANKAIEGIRTRQ |
| | | | VKNMLLTLLLSQGTPMLLSGDEFRRTQGGNNNAYCQDNE |
| | | | LSWLNWTNQETYAEIVTFLRKAIHTRLTHPVFRRPDFFEG |
| | | | QDHSANLLPDINWFASDGKTPDWSQLNHFLAFRLDGSKA |
| | | | EIFADRDDNDFFIMQNTGATDMTVKTPSLNKGKKWYRFID |
| | | | TSVPAPNDFTDHGSEELLEEQKTYILPARTMAVLLGKHHH |
| | | | HHH |
| EAU64550.1 | Stigmatella | 252 | MVEGCSTGERGPPVALFATALGVVPIMMTSIRNLGRAWS |
| | aurantiaca | | ALPWRPLLAVGLGAALSACGASDPAAALLEEGPPVLGGQ |
| | (strain DW4/3-1) | | EQQAVSWTLGARYDASKSNISFQVYSKNATRIDLYIYATA |
| | | | YGAQEVVSYEMTTVPDTGLWSKTVSVATLQNTYKITGPV |
| | | | YYGYRAWGKNWPFSTAWTKGSAAGFIADVDGSGNRFNP |
| | | | NKVLLDPYALEISHDPSNPQSTDGSVFASGPGTRNIDSGP |
| | | | RVPKGIVLAGDSQSIGTRPTRAFKDDVIYEVHVRGLTRND |
| | | | SSIDPAYRGTYKGAGLKAPALAALGVTAVEFLPLHETEND |
| | | | ANDNAVGTPGDNYWGYMSLNYFAPDRRYAYDTKAGGPT |
| | | | REFKEMVKAFHDNGIKVFVDVVYNHTGEGGAWKAGDSS |
| | | | TYNVISFRGLDNATYYSLTGDKQFNWDNTGVGGNYNTYN |
| | | | PKAQDLIIHSLAYWKDTLGVDGFRFDLASVLGNAQEHGGF |
| | | | NFVRDNSNTALNRIIRDLGPRPGSGGAGTDFIAEPWAIGG |
| | | | NSYQVGNFPAGWAEWNGIFRDTFRKDQNQLGVETVTPG |
| | | | QLATRFTGSADLYGEGNGDTRKPYHSVNFMVAHDGFTLK |
| | | | DLYSCNSKNNNQPWPYGPSDGGDDNNHSWDQGGNAAD |
| | | | QRKAARNGFAFLMLSAGVPMFNGGDEFLRSQACNNNAY |
| | | | NLDSDKNWLNYSLSADQTKFKTFAQRLIAFRKAHPALRPA |
| | | | NFYLSTDTNGNVMEQHRWFKPDGYVPDAGYFNNGSNHA |
| | | | IAFRIDGTEFNDSASALYVAYNGWSDNVNFNLPWPGNGK |
| | | | NWYRVADTCPWAETDGVNAVAPGAETLLGGEGYSYGLC |
| | | | GRGVLLLIAK |
| EAU64550.1 | Stigmatella | 253 | MVEGCSTGERGPPVALFATALGVVPIMMTSIRNLGRAWS |
| (6X-His) | aurantiaca | | ALPWRPLLAVGLGAALSACGASDPAAALLEEGPPVLGGQ |
| | (strain DW4/3-1) | | EQQAVSWTLGARYDASKSNISFQVYSKNATRIDLYIYATA |
| | | | YGAQEVVSYEMTTVPDTGLWSKTVSVATLQNTYKITGPV |
| | | | YYGYRAWGKNWPFSTAWTKGSAAGFIADVDGSGNRFNP |
| | | | NKVLLDPYALEISHDPSNPQSTDGSVFASGPGTRNIDSGP |
| | | | RVPKGIVLAGDSQSIGTRPTRAFKDDVIYEVHVRGLTRND |
| | | | SSIDPAYRGTYKGAGLKAPALAALGVTAVEFLPLHETEND |
| | | | ANDNAVGTPGDNYWGYMSLNYFAPDRRYAYDTKAGGPT |
| | | | REFKEMVKAFHDNGIKVFVDVVYNHTGEGGAWKAGDSS |
| | | | TYNVISFRGLDNATYYSLTGDKQFNWDNTGVGGNYNTYN |
| | | | PKAQDLIIHSLAYWKDTLGVDGFRFDLASVLGNAQEHGGF |
| | | | NFVRDNSNTALNRIIRDLGPRPGSGGAGTDFIAEPWAIGG |
| | | | NSYQVGNFPAGWAEWNGIFRDTFRKDQNQLGVETVTPG |
| | | | QLATRFTGSADLYGEGNGDTRKPYHSVNFMVAHDGFTLK |
| | | | DLYSCNSKNNNQPWPYGPSDGGDDNNHSWDQGGNAAD |
| | | | QRKAARNGFAFLMLSAGVPMFNGGDEFLRSQACNNNAY |
| | | | NLDSDKNWLNYSLSADQTKFKTFAQRLIAFRKAHPALRPA |
| | | | NFYLSTDTNGNVMEQHRWFKPDGYVPDAGYFNNGSNHA |
| | | | IAFRIDGTEFNDSASALYVAYNGWSDNVNFNLPWPGNGK |
| | | | NWYRVADTCPWAETDGVNAVAPGAETLLGGEGYSYGLC |
| | | | GRGVLLLIAKHHHHHH |
| KFI71268.1 | Bifidobacterium | 254 | MKNPPLHRYATRPGLYFTDDGGADVVVRSETADQVWLC |
| | longum subsp. | | VLEPIDEPSAFFQDAIRLFEDPNASFIQQIHEFPVCTRIIEHL |
| | suis | | YLRETLFRMTGPNYGLWYVHLPKAWDGMRYGYRVDGA |
| | | | WDPKHGVRFNPYKFLLDPYGKGIDGSMELTPAAFSYECD |
| | | | WVDHKVTGSAYGAMSTVDSLGHVPVSVAIDDRDIHKHEG |
| | | | DPQHPHVPWRKTVIYEMHVKGFTANAPWLPEALRGTYAG |
| | | | LAHPTTLAYLQGLGITSIELLPIMAKQDELFLQEHGRKNYW |
| | | | GYSTLSYFAPEPSYATKAAQEKGAAAVRQEVIDMVRALHE |
| | | | AGFEVIMDVVYNHTCEGGVEGPTVCWRGLDDLAYYRHQ |
| | | | KSNTGRLEDTTGCGNTLDFTNTHVVTFAIDSLRYWAKRIGI |
| | | | DGFRFDLGVTLARLEGEFTHHHPFLYALRSDLLLGNLKLIM |
| | | | EPWDLGNLGWRTGQFSVPFAEWNDRFRDTARTFWLEDV |
| | | | DGGSDFGRISLQEMATRLCGSADLFATEPGRGAPASINFV |
| | | | SCHDGFTLTDLTRYRSKHNEANGENNNDGSSVNHSANF |
| | | | GAEGVTDDPDVITAREQAAMNMIGMLLLSLGTPMMLAGD |
| | | | EFRNTQDGNNNAYCQDNDITWLKWDWMYSTNKTREMR |
| | | | RLKSVSRLVALRKSLDLYHHEDFFTRLTQLGLLKPSSRVQ |
| | | | WFLPDGTTPMERDWFDLGVRSFTMRLLSNSEVDVCIVVN |
| | | | GTADDRTFRLPPDTHWTPKWCSAEINGRRAGHGIQVEEC |
| | | | DLNDDTTVWTQHVPDVSEMVRTLVEEVAMQRTESSTED |
| | | | EADTIEFAMPATDHAANGTSSAPRDEVSADMPDAPVDDT |
| | | | PDTPVDDNVWTMPALSITLMKQV |
| KFI71268.1 | Bifidobacterium | 255 | MKNPPLHRYATRPGLYFTDDGGADVVVRSETADQVWLC |
| (6X-His) | longum subsp. | | VLEPIDEPSAFFQDAIRLFEDPNASFIQQIHEFPVCTRIIEHL |
| | suis | | YLRETLFRMTGPNYGLWYVHLPKAWDGMRYGYRVDGA |
| | | | WDPKHGVRFNPYKFLLDPYGKGIDGSMELTPAAFSYECD |
| | | | WVDHKVTGSAYGAMSTVDSLGHVPVSVAIDDRDIHKHEG |
| | | | DPQHPHVPWRKTVIYEMHVKGFTANAPWLPEALRGTYAG |
| | | | LAHPTTLAYLQGLGITSIELLPIMAKQDELFLQEHGRKNYW |
| | | | GYSTLSYFAPEPSYATKAAQEKGAAAVRQEVIDMVRALHE |
| | | | AGFEVIMDVVYNHTCEGGVEGPTVCWRGLDDLAYYRHQ |
| | | | KSNTGRLEDTTGCGNTLDFTNTHVVTFAIDSLRYWAKRIGI |
| | | | DGFRFDLGVTLARLEGEFTHHHPFLYALRSDLLLGNLKLIM |
| | | | EPWDLGNLGWRTGQFSVPFAEWNDRFRDTARTFWLEDV |
| | | | DGGSDFGRISLQEMATRLCGSADLFATEPGRGAPASINFV |
| | | | SCHDGFTLTDLTRYRSKHNEANGENNNDGSSVNHSANF |
| | | | GAEGVTDDPDVITAREQAAMNMIGMLLLSLGTPMMLAGD |
| | | | EFRNTQDGNNNAYCQDNDITWLKWDWMYSTNKTREMR |
| | | | RLKSVSRLVALRKSLDLYHHEDFFTRLTQLGLLKPSSRVQ |
| | | | WFLPDGTTPMERDWFDLGVRSFTMRLLSNSEVDVCIVVN |
| | | | GTADDRTFRLPPDTHWTPKWCSAEINGRRAGHGIQVEEC |
| | | | DLNDDTTVWTQHVPDVSEMVRTLVEEVAMQRTESSTED |
| | | | EADTIEFAMPATDHAANGTSSAPRDEVSADMPDAPVDDT |
| | | | PDTPVDDNVWTMPALSITLMKQVHHHHHH |
| CCB91631.1 | Waddlia | 256 | MVSLVQTRESSGKSKPYGSKRDSKGVNFAIYSRLATEAAL |
| | chondrophila | | CLFHFDDRRPFKEIPLDPQINRTGYVWHIYVENLPRRLCY |
| | 2032/99 | | AYRFKKGKGKVFTDYYDYQRLVIDPYAKGLATSSVWGEGI |
| | | | GEMPLGLVDEELIFDWEGDRPLNLPREEMMIYEMHIRGFT |
| | | | NHSSSNALWRGKFLGAVEKIPYLKSLGVNAVKLMPINEFN |
| | | | ELEYFRYNPLNGEKLVNYWGYSPLHYFSPMNRYASIDEF |
| | | | GQSILDFKTMVKEFHRNGIEVILDIVLNHTGESDQEPFSFF |
| | | | GIDPQTYYLFDDQHEKMDFTGCGNTINSNHPIVRDFIKDCL |
| | | | RYWVSEMHVDGFRFDLAGVMFRGVHGEPLKNPPLIDAIS |
| | | | NDPILAATKLIAEPWDAAGLYLLGKFYPREERWSEWNDVY |
| | | | RDVVRQFIKGDKGKNRSFATRLCGSDDIFGRSRTPRSSV |
| | | | NFISAHDGFTLRDLVTYNQKDNTSNGENNRDGHPANFSW |
| | | | NCGEEGETDDQEINDLRVRQMKNFHLALMLSQGIPMLLM |
| | | | GNEYGHTRFGNNNSWCQDNEMNWFLWNELELQGDFFR |
| | | | FYRMCIQFRARHPQLRRGRYLTPEDIVWHGKQPDHPDW |
| | | | DGDTQFLAYLLVDEVQSHHLFAAYNPSAENKEVTLPQGE |
| | | | WKLIADTSLSSPDDFRDEEEAPFLPSQEYLLKPYSIALLQC |
| CCB91631.1 | Waddlia | 257 | MVSLVQTRESSGKSKPYGSKRDSKGVNFAIYSRLATEAAL |
| (6X-His) | chondrophila | | CLFHFDDRRPFKEIPLDPQINRTGYVWHIYVENLPRRLCY |
| | 2032/99 | | AYRFKKGKGKVFTDYYDYQRLVIDPYAKGLATSSVWGEGI |
| | | | GEMPLGLVDEELIFDWEGDRPLNLPREEMMIYEMHIRGFT |
| | | | NHSSSNALWRGKFLGAVEKIPYLKSLGVNAVKLMPINEFN |
| | | | ELEYFRYNPLNGEKLVNYWGYSPLHYFSPMNRYASIDEF |
| | | | GQSILDFKTMVKEFHRNGIEVILDIVLNHTGESDQEPFSFF |
| | | | GIDPQTYYLFDDQHEKMDFTGCGNTINSNHPIVRDFIKDCL |
| | | | RYWVSEMHVDGFRFDLAGVMFRGVHGEPLKNPPLIDAIS |
| | | | NDPILAATKLIAEPWDAAGLYLLGKFYPREERWSEWNDVY |
| | | | RDVVRQFIKGDKGKNRSFATRLCGSDDIFGRSRTPRSSV |
| | | | NFISAHDGFTLRDLVTYNQKDNTSNGENNRDGHPANFSW |
| | | | NCGEEGETDDQEINDLRVRQMKNFHLALMLSQGIPMLLM |
| | | | GNEYGHTRFGNNNSWCQDNEMNWFLWNELELQGDFFR |
| | | | FYRMCIQFRARHPQLRRGRYLTPEDIVWHGKQPDHPDW |
| | | | DGDTQFLAYLLVDEVQSHHLFAAYNPSAENKEVTLPQGE |
| | | | WKLIADTSLSSPDDFRDEEEAPFLPSQEYLLKPYSIALLQC |
| | | | HHHHHH |
| CCB89316.1 | Simkania | 258 | MNPSFTTGSKLPLGSSPTEKGINFAVYSHHATNIKLRLFEI |
| | negevensis | | GQKTPFAEFPMERSDDYWHLCVTNLPYTFEYTYQAEGPY |
| | (strain ATCC VR- | | DPSKGLLFCKEMDLVDPYARAVNASDTWGNHHTPMRAL |
| | 1471/Z) | | YETKMPFDWEHTSRPMIPAEDLIIYEMHVRSFTMHPSSGS |
| | | | KNPGTFLGMIEKIPYLKKLGINAVELMPIHEFNETENLRRSP |
| | | | GTGGKLFNYWGYSTSNFFAPMRRFGKEEDLKFLIRELHR |
| | | | EGIEVILDVVYNHTSEGNDQNYYHSFRGLDNPTYYIIDENG |
| | | | YHNYTGCGNTLKCQHPVVQDFILDSLRYWVTEFHVDGFR |
| | | | FDLASIMTRGEDGKPIQDPPLIKRIASDPILAPTKMIAEPWD |
| | | | PAGLYQVGTFPSWRFAEWNGKFRDDVRKFIRGDGNIEAM |
| | | | KNRLLGSPDVYTEKGTPQHSINFITVHDGFTLHDLVSYNE |
| | | | KHNEQNGEQNQDGANDNESWNCGVEGKTTDQAILNLRL |
| | | | QQMRNFMVALFIAQGIPMLLMGDEYAHTREGNNNAYCQD |
| | | | NELNYFLWDQASPLFEFIQKLIALRKSHPIFRQKTFPSAVK |
| | | | WEEKDYLGLTLGEELFIAFNPSAQEYQLEKEGWEILLSTA |
| | | | SQVQTLDKLQPYTSVLFGKKKAL |
| CCB89316.1 | Simkania | 259 | MNPSFTTGSKLPLGSSPTEKGINFAVYSHHATNIKLRLFEI |
| (6X-His) | negevensis | | GQKTPFAEFPMERSDDYWHLCVTNLPYTFEYTYQAEGPY |
| | (strain ATCC VR- | | DPSKGLLFCKEMDLVDPYARAVNASDTWGNHHTPMRAL |
| | 1471/Z) | | YETKMPFDWEHTSRPMIPAEDLIIYEMHVRSFTMHPSSGS |
| | | | KNPGTFLGMIEKIPYLKKLGINAVELMPIHEFNETENLRRSP |
| | | | GTGGKLFNYWGYSTSNFFAPMRRFGKEEDLKFLIRELHR |
| | | | EGIEVILDVVYNHTSEGNDQNYYHSFRGLDNPTYYIIDENG |
| | | | YHNYTGCGNTLKCQHPVVQDFILDSLRYWVTEFHVDGFR |
| | | | FDLASIMTRGEDGKPIQDPPLIKRIASDPILAPTKMIAEPWD |
| | | | PAGLYQVGTFPSWRFAEWNGKFRDDVRKFIRGDGNIEAM |
| | | | KNRLLGSPDVYTEKGTPQHSINFITVHDGFTLHDLVSYNE |
| | | | KHNEQNGEQNQDGANDNESWNCGVEGKTTDQAILNLRL |
| | | | QQMRNFMVALFIAQGIPMLLMGDEYAHTREGNNNAYCQD |
| | | | NELNYFLWDQASPLFEFIQKLIALRKSHPIFRQKTFPSAVK |
| | | | WEEKDYLGLTLGEELFIAFNPSAQEYQLEKEGWEILLSTA |
| | | | SQVQTLDKLQPYTSVLFGKKKALHHHHHH |
| ADY61061.1 | Rubinisphaera | 260 | MRSWHTMEGWHDPPGVSWIEEEKAWNFVLYAREATRVV |
| | brasiliensis | | LLIYSREDQTKPILEYQFDQYRNKSGPVWHCRISHDRAPK |
| | (strain ATCC | | AHYYAYRVSGPLHEEDKFNRFDPEKILLDPYAKAVYFPPG |
| | 49424/DSM | | FDREVAKHPGPNDGKAPLGVIDAEIHFDWKDDRFIRHDSD |
| | 5305/JCM | | LVIYEMHVRGFTKHSSSQVPTAKRGTYAGVIEKIPYLRDLG |
| | 21570/IAM | | VTAVELMPIFQYDPQELNYWGYMPLNFFAPHCDYAVNSE |
| | 15109/NBRC | | SQQVREEFREMVQELHRAGIEVILDVVYNHTCEGDDNGP |
| | 103401/IFAM | | CYSFKGIGNSMYYMEAPDENGEPTFANYSGCGNTLNANT |
| | 1448) | | LAVRKLVVDSLKYWRDEMHVDGFRFDLASIFARQNDGTIS |
| | | | AGQTPIFSQIVTAEDFLNVRLIAEPWDAAGTYQLGHSFPG |
| | | | WLWMQWNGRYRDTMQQFVAGQPGMIGDLMSRLYGSAD |
| | | | LFPDDLNHSCRPWQSVNYITSHDGSTLYDMVTYEGKYNW |
| | | | DNGEENRDGSHEFKWNCGHEGEEGTPPEVMRLRKRQV |
| | | | KNFMTLLMLSNGSAMFRMGDEFMATQKGNNNAYNQDNE |
| | | | TSWLDWTRLDRFRDVYRFVKLLIAFRKSHPSLSRSHYWR |
| | | | NDIRWYGPDGPCDISHNSHTLAYSLRGSSVNDQDLYVMI |
| | | | NGSREPRSFEICDGAETQWKRILDTALDSPDDIHGVSGYE |
| | | | TLNQTSYHLEANSIVVLVRDA |
| ADY61061.1 | Rubinisphaera | 261 | MRSWHTMEGWHDPPGVSWIEEEKAWNFVLYAREATRVV |
| (6X-His) | brasiliensis | | LLIYSREDQTKPILEYQFDQYRNKSGPVWHCRISHDRAPK |
| | (strain ATCC | | AHYYAYRVSGPLHEEDKFNRFDPEKILLDPYAKAVYFPPG |
| | 49424/DSM | | FDREVAKHPGPNDGKAPLGVIDAEIHFDWKDDRFIRHDSD |
| | 5305/JCM | | LVIYEMHVRGFTKHSSSQVPTAKRGTYAGVIEKIPYLRDLG |
| | 21570/IAM | | VTAVELMPIFQYDPQELNYWGYMPLNFFAPHCDYAVNSE |
| | 15109/NBRC | | SQQVREEFREMVQELHRAGIEVILDVVYNHTCEGDDNGP |
| | 103401/IFAM | | CYSFKGIGNSMYYMEAPDENGEPTFANYSGCGNTLNANT |
| | 1448) | | LAVRKLVVDSLKYWRDEMHVDGFRFDLASIFARQNDGTIS |
| | | | AGQTPIFSQIVTAEDFLNVRLIAEPWDAAGTYQLGHSFPG |
| | | | WLWMQWNGRYRDTMQQFVAGQPGMIGDLMSRLYGSAD |
| | | | LFPDDLNHSCRPWQSVNYITSHDGSTLYDMVTYEGKYNW |
| | | | DNGEENRDGSHEFKWNCGHEGEEGTPPEVMRLRKRQV |
| | | | KNFMTLLMLSNGSAMFRMGDEFMATQKGNNNAYNQDNE |
| | | | TSWLDWTRLDRFRDVYRFVKLLIAFRKSHPSLSRSHYWR |
| | | | NDIRWYGPDGPCDISHNSHTLAYSLRGSSVNDQDLYVMI |
| | | | NGSREPRSFEICDGAETQWKRILDTALDSPDDIHGVSGYE |
| | | | TLNQTSYHLEANSIVVLVRDAHHHHHH |
| ADZ83787.1 | Cellulosilyticum | 262 | MYGSSVGQSVIGCHFTEMRTHLGVYTTIASKMLLEIYEEA |
| | lentocellum | | RSIVPITKVIMDAATYQKDNVFSIQIEGLKEGMAYVWRIVH |
| | (strain ATCC | | EDYSYSPSIMDPYAKGTFFFQGEWRNIIKKTNRYHLPKPQI |
| | 49066/DSM | | PWEETILYEMHVGHFTMNNKTLSNEKRGTFIGLMQQLPYL |
| | 5427/NCIMB | | KQLGVTTLELLPIFKWNAYTLKNRNPQTGELLEDVWGYNP |
| | 11756/RHM5) | | VGFFCVDERFSVSKESSKAIDEFKLLVEKAHEEGLEIILDVV |
| | | | YNHTGEGGEDGTAFHFKYLAPKVYYKYNDKGQFLNCSGT |
| | | | GNTLNTNHSIVKKFIIDSLVYWSEEVGVDGFRFDLASILGQ |
| | | | DECGRWIKTSLLNEIAEHPLLGKVKLISESWDAKGSYDVG |
| | | | RMPYPFREWSDYFRDTMRQFVKGDQGKIKALADCIQGKE |
| | | | VYFTDLTKGTSHMIHFITAHDGFTMWDLLSYNDKHNEANG |
| | | | EGNRDGHNANYSYNWGVEGTTEDTNILEARKRGMRNLM |
| | | | CLLILAQGVPMLLMGDEIGRTQGGNNNAFCQNNESVWM |
| | | | DWERVTTFKSQYLFVQRLIALRKSLDYFKTADKNNYKISW |
| | | | HGIRYNQPDWSYYSKSIACFIEGDQSLFLVANSHYESLRF |
| | | | EMPPSNKKWCRVIDTAYKEIEDIINEEIIEETNYEVKPYSICF |
| | | | FKEINHKKIG |
| ADZ83787.1 | Cellulosilyticum | 263 | MYGSSVGQSVIGCHFTEMRTHLGVYTTIASKMLLEIYEEA |
| (6X-His) | lentocellum | | RSIVPITKVIMDAATYQKDNVFSIQIEGLKEGMAYVWRIVH |
| | (strain ATCC | | EDYSYSPSIMDPYAKGTFFFQGEWRNIIKKTNRYHLPKPQI |
| | 49066/DSM | | PWEETILYEMHVGHFTMNNKTLSNEKRGTFIGLMQQLPYL |
| | 5427/NCIMB | | KQLGVTTLELLPIFKWNAYTLKNRNPQTGELLEDVWGYNP |
| | 11756/RHM5) | | VGFFCVDERFSVSKESSKAIDEFKLLVEKAHEEGLEIILDVV |
| | | | YNHTGEGGEDGTAFHFKYLAPKVYYKYNDKGQFLNCSGT |
| | | | GNTLNTNHSIVKKFIIDSLVYWSEEVGVDGFRFDLASILGQ |
| | | | DECGRWIKTSLLNEIAEHPLLGKVKLISESWDAKGSYDVG |
| | | | RMPYPFREWSDYFRDTMRQFVKGDQGKIKALADCIQGKE |
| | | | VYFTDLTKGTSHMIHFITAHDGFTMWDLLSYNDKHNEANG |
| | | | EGNRDGHNANYSYNWGVEGTTEDTNILEARKRGMRNLM |
| | | | CLLILAQGVPMLLMGDEIGRTQGGNNNAFCQNNESVWM |
| | | | DWERVTTFKSQYLFVQRLIALRKSLDYFKTADKNNYKISW |
| | | | HGIRYNQPDWSYYSKSIACFIEGDQSLFLVANSHYESLRF |
| | | | EMPPSNKKWCRVIDTAYKEIEDIINEEIIEETNYEVKPYSICF |
| | | | FKEINHKKIGHHHHHH |
| EGV32209.1 | Thiorhodococcus | 264 | MRSPRYRVRPGSWDTAGATVSNDGVNFCVFSRYAERMS |
| | drewsii AZ1 | | LLLFERETSKEPYEVLHLNPRINRTFFFWHIFVENLPDGTY |
| | | | YNWRATGPGDTSETGSRLDSEKALLDPWATTISDRLWDR |
| | | | ATACRPGNNVAKAMRAQVIRDDYDWEEDQPLYVSLNNAII |
| | | | YEMHVGGFTRHPSSGVVHPGTFEGVTEKIPYLQDLGITHV |
| | | | ELMPVMAFDPQDVPPQTAKMGMENYWGYSTHSFFAPHP |
| | | | GFAVTAINARDEFRDMVKAFHRAGIGVILDVVFNHTAEGG |
| | | | KNGPVISFKAFGNEIFYHLDFEDRSLYRDYTGCGNTMNCN |
| | | | HPMVTRFLIDALLYWVRHMHVDGFRFDLASALARGEDGN |
| | | | PHHHAPVLWAIELSPSLNRAHIIAEAWDAAGLYQVGDFPG |
| | | | YRWAEWNGRYRDVIRSFVRGDAGMVPEVATRMSGSSD |
| | | | MYEGRGRLPMNSINFITCHDGFTLCDLVSYNAKHNEANG |
| | | | EDNRDGHDHNLSWNCGIEGPTDDPEIQALRRRQARNFISI |
| | | | LMLSQGVPMLLSGDEVFRSKSGNNNTYCQNNELSWSDW |
| | | | DLVDTNSEMLEFVRSMIALRRRHPTLTRDRFLRGKPDYG |
| | | | HSRPDIIWHGVDLEQPNWTDATSRQLAFTLEGSVDDEQP |
| | | | LHVMLNMGSEPIAFALPPIAGLQWGLALDTSSERPAVEPA |
| | | | DQRPIAEDRLQLGPFSVVVLEARPA |
| EGV32209.1 | Thiorhodococcus | 265 | MRSPRYRVRPGSWDTAGATVSNDGVNFCVFSRYAERMS |
| (6X-His) | drewsii AZ1 | | LLLFERETSKEPYEVLHLNPRINRTFFFWHIFVENLPDGTY |
| | | | YNWRATGPGDTSETGSRLDSEKALLDPWATTISDRLWDR |
| | | | ATACRPGNNVAKAMRAQVIRDDYDWEEDQPLYVSLNNAII |
| | | | YEMHVGGFTRHPSSGVVHPGTFEGVTEKIPYLQDLGITHV |
| | | | ELMPVMAFDPQDVPPQTAKMGMENYWGYSTHSFFAPHP |
| | | | GFAVTAINARDEFRDMVKAFHRAGIGVILDVVFNHTAEGG |
| | | | KNGPVISFKAFGNEIFYHLDFEDRSLYRDYTGCGNTMNCN |
| | | | HPMVTRFLIDALLYWVRHMHVDGFRFDLASALARGEDGN |
| | | | PHHHAPVLWAIELSPSLNRAHIIAEAWDAAGLYQVGDFPG |
| | | | YRWAEWNGRYRDVIRSFVRGDAGMVPEVATRMSGSSD |
| | | | MYEGRGRLPMNSINFITCHDGFTLCDLVSYNAKHNEANG |
| | | | EDNRDGHDHNLSWNCGIEGPTDDPEIQALRRRQARNFISI |
| | | | LMLSQGVPMLLSGDEVFRSKSGNNNTYCQNNELSWSDW |
| | | | DLVDTNSEMLEFVRSMIALRRRHPTLTRDRFLRGKPDYG |
| | | | HSRPDIIWHGVDLEQPNWTDATSRQLAFTLEGSVDDEQP |
| | | | LHVMLNMGSEPIAFALPPIAGLQWGLALDTSSERPAVEPA |
| | | | DQRPIAEDRLQLGPFSVVVLEARPAHHHHHH |
| AFY71689.1 | Pseudanabaena | 266 | MVLDRIDIHPTHEYKGFKLRYGRPFPFGATLVPGGVNFSIF |
| | sp. PCC 7367 | | SRHATACTLVLFERHAKEPFAEIPFLEEFRIGNVFTMTVFD |
| | | | LNYEELEYGYRMDGPYDPKEGHWFDKTKILMDPYARIIGG |
| | | | RDIWGKQPDWDDVYHHRARIAFDDFDWESDRPLEIPPED |
| | | | LTVYEMHVRSFTKHESSGVKHPGTYAAIRDKIPYLKELGV |
| | | | NCIELMPIYEFDEFENSRPNPQNPEETLMNYWGYSTVGFF |
| | | | APKAGYAATGKFGMQVDELKAMIKELHRNGIEVILDVVFN |
| | | | HTAEGNEKGPYISYRGLDNQTYYMLTPDGYYFNFSGTGN |
| | | | TLNCNNPIVRNMVLDCLRYWAAEYHVDGFRFDLASILSRD |
| | | | AWGAPLANPPLLETLAFDPILAKCKLIAEAWDAGGLYQVG |
| | | | SFPAFGRWAEWNGKYRDCIRKFIKGDDGMAGEMAQRIQ |
| | | | GSPDLYAWGGRGPATSINFITCHDGFTLMDTVSYNGKHN |
| | | | DANGEDNRDGNNDNDSWNCGWEGPTDDSGINALRQRQI |
| | | | KNAVAIMIASQGVPMFLMGDEMGRTKYGNNNTYCHDNEL |
| | | | NWLDWDLLNQNQGLFRFFKLAIAFRMAHPVLRSRTHFRN |
| | | | YDYVGSGYSDITWHGTQAWNADWSDSSHALAFMLCGKH |
| | | | AKEGTVNDDYIYVAMNTHWDALWFEPPGLPEGMQWHVF |
| | | | ANTGASSPEDVWTPGQEPALENQHGILLGDRSVVILVGR |
| AFY71689.1 | Pseudanabaena | 267 | MVLDRIDIHPTHEYKGFKLRYGRPFPFGATLVPGGVNFSIF |
| (6X-His) | sp. PCC 7367 | | SRHATACTLVLFERHAKEPFAEIPFLEEFRIGNVFTMTVFD |
| | | | LNYEELEYGYRMDGPYDPKEGHWFDKTKILMDPYARIIGG |
| | | | RDIWGKQPDWDDVYHHRARIAFDDFDWESDRPLEIPPED |
| | | | LTVYEMHVRSFTKHESSGVKHPGTYAAIRDKIPYLKELGV |
| | | | NCIELMPIYEFDEFENSRPNPQNPEETLMNYWGYSTVGFF |
| | | | APKAGYAATGKFGMQVDELKAMIKELHRNGIEVILDVVFN |
| | | | HTAEGNEKGPYISYRGLDNQTYYMLTPDGYYFNFSGTGN |
| | | | TLNCNNPIVRNMVLDCLRYWAAEYHVDGFRFDLASILSRD |
| | | | AWGAPLANPPLLETLAFDPILAKCKLIAEAWDAGGLYQVG |
| | | | SFPAFGRWAEWNGKYRDCIRKFIKGDDGMAGEMAQRIQ |
| | | | GSPDLYAWGGRGPATSINFITCHDGFTLMDTVSYNGKHN |
| | | | DANGEDNRDGNNDNDSWNCGWEGPTDDSGINALRQRQI |
| | | | KNAVAIMIASQGVPMFLMGDEMGRTKYGNNNTYCHDNEL |
| | | | NWLDWDLLNQNQGLFRFFKLAIAFRMAHPVLRSRTHFRN |
| | | | YDYVGSGYSDITWHGTQAWNADWSDSSHALAFMLCGKH |
| | | | AKEGTVNDDYIYVAMNTHWDALWFEPPGLPEGMQWHVF |
| | | | ANTGASSPEDVWTPGQEPALENQHGILLGDRSVVILVGR |
| | | | HHHHHH |
| ABX09307.1 | Prochlorococcus | 268 | MGKIHKGSPWPLGSSITSRGVNFSVASPEASYIELLIFKNE |
| | marinus (strain | | DDLQPKKILTLDKNHRSGDYWHIEVEGINTGCFYCYRVIG |
| | MIT 9211) | | NRSTCKQDIFSRKILLDPCSRGIAGWNIFQRESATGLSTNI |
| | | | DKCLKSIVTERDQFDFQSYPRPKHSWDKTIIYELHVGGFT |
| | | | KSSESDVKDKIKGTFLGLIEKIPYLKQLGVTTLELLPVFAFD |
| | | | TTDSPYGLNNYWGYSPINWFTPHHSFIASNNPINARDQFR |
| | | | NFIKVCHKNDLEVILDVVYNHTTEGNEKGPIISWKGFAEST |
| | | | YYHQNKEGKYLDVTGCGNTIAANNPLVRQLILESMRCWA |
| | | | NELGVDGFRFDLGISLSRGKDLKPLDSPPLFEEIESDPALS |
| | | | DLKLISEPWDCGGLYRLSDFPAKRCCTWNGHFRDDIRRF |
| | | | WNGDKNSTWPLKDRLTGSPELYKDNFKSAQKSINFITSHD |
| | | | GFTLKDLVSFNLKHNLSNGESNRDGENHNNSCNNGIEGP |
| | | | TTNKKVNLIRSKNQRNLIATLLLSPGIPMILMGDEVGRSQG |
| | | | GNNNAWCQDNPLGWMIWRTDNCDNELRSFVSMCIYIRK |
| | | | ELSDFFAPLININSDSPSLQSQEKLWVQWHGVKINAPDWG |
| | | | SWSNTIGFSINKAKEGAIIWMGFNAFNQSMKFELPKPLSP |
| | | | WVKILDTTLLTQKEYGLFRLSNQLEIEIESKSLVVLVAKEYT |
| | | | KKLRI |
| ABX09307.1 | Prochlorococcus | 269 | MGKIHKGSPWPLGSSITSRGVNFSVASPEASYIELLIFKNE |
| (6X-His) | marinus (strain | | DDLQPKKILTLDKNHRSGDYWHIEVEGINTGCFYCYRVIG |
| | MIT 9211) | | NRSTCKQDIFSRKILLDPCSRGIAGWNIFQRESATGLSTNI |
| | | | DKCLKSIVTERDQFDFQSYPRPKHSWDKTIIYELHVGGFT |
| | | | KSSESDVKDKIKGTFLGLIEKIPYLKQLGVTTLELLPVFAFD |
| | | | TTDSPYGLNNYWGYSPINWFTPHHSFIASNNPINARDQFR |
| | | | NFIKVCHKNDLEVILDVVYNHTTEGNEKGPIISWKGFAEST |
| | | | YYHQNKEGKYLDVTGCGNTIAANNPLVRQLILESMRCWA |
| | | | NELGVDGFRFDLGISLSRGKDLKPLDSPPLFEEIESDPALS |
| | | | DLKLISEPWDCGGLYRLSDFPAKRCCTWNGHFRDDIRRF |
| | | | WNGDKNSTWPLKDRLTGSPELYKDNFKSAQKSINFITSHD |
| | | | GFTLKDLVSFNLKHNLSNGESNRDGENHNNSCNNGIEGP |
| | | | TTNKKVNLIRSKNQRNLIATLLLSPGIPMILMGDEVGRSQG |
| | | | GNNNAWCQDNPLGWMIWRTDNCDNELRSFVSMCIYIRK |
| | | | ELSDFFAPLININSDSPSLQSQEKLWVQWHGVKINAPDWG |
| | | | SWSNTIGFSINKAKEGAIIWMGFNAFNQSMKFELPKPLSP |
| | | | WVKILDTTLLTQKEYGLFRLSNQLEIEIESKSLVVLVAKEYT |
| | | | KKLRIHHHHHH |
| KNY26877.1 | Pseudo- | 270 | MQIEMGSSYDKDTGKVHFNVFSKNASHIEIYFYLTPFGAD |
| | bacteroides | | EVMKKSLVRNNDIWSLDLPLAELKSAGFTSNCVYYGYRT |
| | cellulosolvens | | WGPNWEYMEDWNKTSDKGFICDVDDEGNRFNPNKLLID |
| | ATCC 35603 = | | PYSKELSHDPQVAKIYIDPNVYIDNYYSGANRNADTGKIAP |
| | DSM 2933 | | KSMLFLEKDKTSYGIKPKRHLKDDIIYEVNLRGLTMMDKSI |
| | | | PKDERGTYKAASIKAAYLKELGVTAVEFLPIHEFADEQNDD |
| | | | NDPRGDNYWGYMTLNFFSPNRRYSFDKSPGGPTREFKQ |
| | | | MIKAFHEQGIKVFLDVVYNHTGEGILKRIINQGNPSTEEDIK |
| | | | KAIQEANHSRGDDNLQDYTAACIMSFTGLDNSNYYYLRD |
| | | | GNKRYEGRGGCGGNLKYDNEVVRKLIYDSLKYWKDEMG |
| | | | VDGFRFDLAPVLSVTGSNGNYWPDTNTTIFEEISNILPSRI |
| | | | KDQDNGADLIAEPWGEGSSIDWLDKFPNSWAVWNKAFR |
| | | | DIFKTSMNKYGVAPFRICNIANVSSGSSSIIKKKPWNSINYF |
| | | | VSHDDCNSLRNIFSYNEYFHLNEAGIKNDQITWNQGNDPS |
| | | | LQKKAVLNAFTLMMFSAGVPMFTGGDEFYRAISPYHQGV |
| | | | GRMNMVTVDGPDGYVDFQHFNKLTVLRNSGNSHEAEQL |
| | | | TKNTDELYIFEFVKKVIDFRSNHECLRPENYFKGERENDN |
| | | | FLKDITWYQHNGLEIDSSYWDSSDFVAYRISAESEKVANV |
| | | | NNRIFSIYLAYNRSPRTEKVLLPSNIPNKRWYRLIDTDNTY |
| | | | GWMSELRNFDGDTLLENEYVMHDRSILVLIEK |
| KNY26877.1 | Pseudo- | 271 | MQIEMGSSYDKDTGKVHFNVFSKNASHIEIYFYLTPFGAD |
| (6X-His) | bacteroides | | EVMKKSLVRNNDIWSLDLPLAELKSAGFTSNCVYYGYRT |
| | cellulosolvens | | WGPNWEYMEDWNKTSDKGFICDVDDEGNRFNPNKLLID |
| | ATCC 35603 = | | PYSKELSHDPQVAKIYIDPNVYIDNYYSGANRNADTGKIAP |
| | DSM 2933 | | KSMLFLEKDKTSYGIKPKRHLKDDIIYEVNLRGLTMMDKSI |
| | | | PKDERGTYKAASIKAAYLKELGVTAVEFLPIHEFADEQNDD |
| | | | NDPRGDNYWGYMTLNFFSPNRRYSFDKSPGGPTREFKQ |
| | | | MIKAFHEQGIKVFLDVVYNHTGEGILKRIINQGNPSTEEDIK |
| | | | KAIQEANHSRGDDNLQDYTAACIMSFTGLDNSNYYYLRD |
| | | | GNKRYEGRGGCGGNLKYDNEVVRKLIYDSLKYWKDEMG |
| | | | VDGFRFDLAPVLSVTGSNGNYWPDTNTTIFEEISNILPSRI |
| | | | KDQDNGADLIAEPWGEGSSIDWLDKFPNSWAVWNKAFR |
| | | | DIFKTSMNKYGVAPFRICNIANVSSGSSSIIKKKPWNSINYF |
| | | | VSHDDCNSLRNIFSYNEYFHLNEAGIKNDQITWNQGNDPS |
| | | | LQKKAVLNAFTLMMFSAGVPMFTGGDEFYRAISPYHQGV |
| | | | GRMNMVTVDGPDGYVDFQHFNKLTVLRNSGNSHEAEQL |
| | | | TKNTDELYIFEFVKKVIDFRSNHECLRPENYFKGERENDN |
| | | | FLKDITWYQHNGLEIDSSYWDSSDFVAYRISAESEKVANV |
| | | | NNRIFSIYLAYNRSPRTEKVLLPSNIPNKRWYRLIDTDNTY |
| | | | GWMSELRNFDGDTLLENEYVMHDRSILVLIEKHHHHHH |
| KXK30828.1 | Candidatus | 272 | MDPVTFTTGNAVIDRTRGNSFPLGATVLQGGVNFSVFSK |
| | Brocadia sinica | | SATFVELLLFDHAEDPGPSRTILLDPRKNRTYHYWHVFVP |
| | | | DVGPGQIYGYRVHGPYEPARGMRFDPGKVLLDPYGRAV |
| | | | WVPEKYSRTAAGLPGDNCGAAMKSVVTDPRTYDWEGDL |
| | | | PLRRPFTRTIIYEMHVRGFTAHPNSGVAPEKRGTYAGLIEK |
| | | | IPYLKDLGITAVELLPVYHFDEQDAPPEFKNYWGYAPVSFF |
| | | | TPHPAYSSRKEPLCVVDEFRDMIKALHRAGIEVILDVVYNH |
| | | | TAEDNHEGPTLSFRGFENDAYYILGPDKRHYSNYTGCGN |
| | | | TLNASNPFVRRMIIDSLHYWVQEMHVDGFRFDLASILARD |
| | | | EQGRPLANPPVLWDIETDPVLSGVKLIAEAWDAAGLYQV |
| | | | GSFIGDSWKEWNVRFRDDVRSFLKGDRNTVSKFASRLLG |
| | | | SPDIYGHEEREPEQSINFVTCHDGFTLNDLVSYNDKHNEE |
| | | | NGEENRDGSNDNMSWNCGIEGPTDALPIERLRNRQVKNF |
| | | | FAVTLLAAGAPMLLMGDEVRRTQRGNNNAYGQDNEISWF |
| | | | DWGLVEKHANVLRFVKYLITARLRRNVSLEDLGLTLNQLL |
| | | | SQAKITWHGVKLNQPDWGADSHAVALTARSLKGRFIIHTM |
| | | | INAYWEGLEFDVPPVSEFECEVWMRWIDTARESPDDISS |
| | | | WDEASVVREAVYPVQPRSLVVLVSRVKNRGKL |
| KXK30828.1 | Candidatus | 273 | MDPVTFTTGNAVIDRTRGNSFPLGATVLQGGVNFSVFSK |
| (6X-His) | Brocadia sinica | | SATFVELLLFDHAEDPGPSRTILLDPRKNRTYHYWHVFVP |
| | | | DVGPGQIYGYRVHGPYEPARGMRFDPGKVLLDPYGRAV |
| | | | WVPEKYSRTAAGLPGDNCGAAMKSVVTDPRTYDWEGDL |
| | | | PLRRPFTRTIIYEMHVRGFTAHPNSGVAPEKRGTYAGLIEK |
| | | | IPYLKDLGITAVELLPVYHFDEQDAPPEFKNYWGYAPVSFF |
| | | | TPHPAYSSRKEPLCVVDEFRDMIKALHRAGIEVILDVVYNH |
| | | | TAEDNHEGPTLSFRGFENDAYYILGPDKRHYSNYTGCGN |
| | | | TLNASNPFVRRMIIDSLHYWVQEMHVDGFRFDLASILARD |
| | | | EQGRPLANPPVLWDIETDPVLSGVKLIAEAWDAAGLYQV |
| | | | GSFIGDSWKEWNVRFRDDVRSFLKGDRNTVSKFASRLLG |
| | | | SPDIYGHEEREPEQSINFVTCHDGFTLNDLVSYNDKHNEE |
| | | | NGEENRDGSNDNMSWNCGIEGPTDALPIERLRNRQVKNF |
| | | | FAVTLLAAGAPMLLMGDEVRRTQRGNNNAYGQDNEISWF |
| | | | DWGLVEKHANVLRFVKYLITARLRRNVSLEDLGLTLNQLL |
| | | | SQAKITWHGVKLNQPDWGADSHAVALTARSLKGRFIIHTM |
| | | | INAYWEGLEFDVPPVSEFECEVWMRWIDTARESPDDISS |
| | | | WDEASVVREAVYPVQPRSLVVLVSRVKNRGKLHHHHHH |
| OQB68359.1 | Spirochaetes | 274 | MKKFLFILLLIIAMLGCVQSGIFNNAGNNKGNTISDAKYGTL |
| | bacterium | | VIKNIDNGKSTNIDPTRSVVISDIKTAMVTVSGRGMNDISES |
| | ADurb.Bin133 | | AAVVNGAGEIRVENIPIGKNRVISVQARTDSGDMTGIVMR |
| | | | GIKDIVSGDNTVNIRWSSTPLGDTFYELLYTHDYDIGSMSD |
| | | | ATRSNITAIISGTGVSHPSLFNSKNLAEDVANGTMKSANDA |
| | | | SYKITPASAVFSINGITNFTGFTAQVNDPASAKLSSSTTPIT |
| | | | TGENTITGIKPGNWELVITIDGGIVYRSGVVALSSGMPYNF |
| | | | GAIDLLVSDPYFLPVNTTFAASLDVTIDVSTTGASIYYTTNG |
| | | | VDPTESDTKYTAPIKITGTTTFKARAFKTNYLSSNVITKTYT |
| | | | KIISSAIGENHPSTGAFSPMDLNGETAGFGWATQTWDLGS |
| | | | HFAGTDVTFAVYSKNATKVLLEIYETETSSTNTNKAYGTDA |
| | | | RYDYWMEKGSDNIWRAKIAAVPEKTYYAFRVWGPNWTF |
| | | | NSSWTRGNSSAGFLADVDANGNRFNPNKVVFDPYTREIS |
| | | | HDKEFPAMAAAGENGGMYGTSGAEIDAEHIYSGPTTTGG |
| | | | VSINRRNVDTGRWVPKGIVLKPTGKTFTKPTFAQEASVIYE |
| | | | AHVRGITAHSSSSNLETILNGMEGFATVVNVPSQYRGTYK |
| | | | GVAYLAKYLKAIGINTIELLPVHETENDMLPFDGSQPTLGG |
| | | | AKNFWGYMTYGFFAPDRRYSYDKSAGGPTSEFQDMVQA |
| | | | LNAEGIKVFLDVVFNHTGEGGNWGHMNVTGFVSMGGLD |
| | | | CAEYYHLIPGGDAKNWLVDGATGCGNQLNFSKTVNHNLI |
| | | | MDSLTYWIDKMGISGYRFDLAAVIGRKPDLHAWEPSDTY |
| | | | WDRVKAFYSDHPTLTAMASLGASKSA |
| OQB68359.1 | Spirochaetes | 275 | CVQSGIFNNAGNNKGNTISDAKYGTLVIKNIDNGKSTNIDP |
| (6X-His) | bacterium | | TRSVVISDIKTAMVTVSGRGMNDISESAAVVNGAGEIRVE |
| | ADurb.Bin133 | | NIPIGKNRVISVQARTDSGDMTGIVMRGIKDIVSGDNTVNI |
| | | | RWSSTPLGDTFYELLYTHDYDIGSMSDATRSNITAIISGTG |
| | | | VSHPSLFNSKNLAEDVANGTMKSANDASYKITPASAVFSI |
| | | | NGITNFTGFTAQVNDPASAKLSSSTTPITTGENTITGIKPGN |
| | | | WELVITIDGGIVYRSGVVALSSGMPYNFGAIDLLVSDPYFL |
| | | | PVNTTFAASLDVTIDVSTTGASIYYTTNGVDPTESDTKYTA |
| | | | PIKITGTTTFKARAFKTNYLSSNVITKTYTKIISSAIGENHPS |
| | | | TGAFSPMDLNGETAGFGWATQTWDLGSHFAGTDVTFAV |
| | | | YSKNATKVLLEIYETETSSTNTNKAYGTDARYDYWMEKGS |
| | | | DNIWRAKIAAVPEKTYYAFRVWGPNWTFNSSWTRGNSSA |
| | | | GFLADVDANGNRFNPNKVVFDPYTREISHDKEFPAMAAA |
| | | | GENGGMYGTSGAEIDAEHIYSGPTTTGGVSINRRNVDTG |
| | | | RWVPKGIVLKPTGKTFTKPTFAQEASVIYEAHVRGITAHSS |
| | | | SSNLETILNGMEGFATVVNVPSQYRGTYKGVAYLAKYLKA |
| | | | IGINTIELLPVHETENDMLPFDGSQPTLGGAKNFWGYMTY |
| | | | GFFAPDRRYSYDKSAGGPTSEFQDMVQALNAEGIKVELD |
| | | | VVFNHTGEGGNWGHMNVTGFVSMGGLDCAEYYHLIPGG |
| | | | DAKNWLVDGATGCGNQLNFSKTVNHNLIMDSLTYWIDKM |
| | | | GISGYRFDLAAVIGRKPDLHAWEPSDTYWDRVKAFYSDH |
| | | | PTLTAMASLGASKSAHHHHHH |
| OQB68755.1 | Spirochaetes | 276 | MILKKIKEYSKTFTFAIIIVALVVSFGCSNHSSLKYDNEDQD |
| | bacterium | | FAYEKGYGLLEVNIPTVRGWTVAQYDVVATKPGETSVTAS |
| | ADurb. Bin133 | | TASTSVSLRLKIGTWTISVVGKDSYANTIYQGVASANVTES |
| | | | GNSVTIGLLKKAGNYKLTLNSTYPVVSGQPGYIEKIVVTAS |
| | | | RGQGFADVVAESNAFSNSFIFSGLAQGNWTFTARGYAAG |
| | | | LDSDYQPTGTEVLFLEDSYTLNVVASKITSSTQNLNTQKK |
| | | | ATPVKFSHLAGTYSGTINVSLSCDTAGATIYYTTDGSNPTT |
| | | | SGTKKTYSTSVAISATTTLKVAAVKSGLTSSIVGSRLYTIDA |
| | | | GVTSTPQMNPTGGTYNSDQNVVLSCADAGAIIYYTTDGS |
| | | | NPTTSSTQYSAPIAVSGNGTTKVIRAIAKAGAKNVSGAAS |
| | | | QSYTISYLASSAPTFTPASGNLTTNDNITIECGTSGASIYYT |
| | | | TDGSDPKISGTRTPYTSPFSLAVGTYTVKAYSTASGYADS |
| | | | TVTSGEFNVTQPQQDNSIIFATAETIISGEEWNPSSLAAGI |
| | | | NLFGVDIFDTGTFKLEWSETYSGSVTMYENDLTTPLSLTG |
| | | | TGPSRTVSLVSGERYFINFNSTYEMANFSVRVYLGASGG |
| | | | GGALGSNYPSSGSYSPVNMADWGTATWELGANYVSGPP |
| | | | SNLRIGVFAANATKVLLEIYSQKTGQTAQYDYWMAKGSD |
| | | | GVWRAELNSVPNYALYAFRAWGPNWSFDSNWTRGNSA |
| | | | SGFTSDVDAAGNRYNPNKVLYDPYAKEISHDKETPEMAA |
| | | | AGENGGMYGTGGLTDLTPHGYRGPCTNNVNIDIRNVDTG |
| | | | KWAPKSVAVYNTTSFGTKPNIAEKDGIIYEAHVRGLTQHS |
| | | | SSISLPSILSGVGINTSSWGTWFTDAERGTYKAAGKMAKY |
| | | | LKALGYTTIELLPVHETANDINPADSPGGNYWGYMTYGYF |
| | | | APDRRYSYDKTFGGPTKEFKEMCAAFHAEGLEVYLDVVY |
| | | | NHTGEGGNWDPSHIESPVGSGNWIENPDFTTKCKEITSF |
| | | | AGLDNANYYALVSTDKGRYWETTGCGNNMDVSKAVVRD |
| | | | LIKDSLKYWITEMGVDGFRFDLAPVLGRDAAPNYHFNPNG |
| | | | QLLTEIAAMGATYNVEMIAEAWDCQWPGGYQVSNFPAG |
| | | | WGEWNGFYRDSVRKFIKGGGNKTSGYPSFTDAFNGSYG |
| | | | PLDRSSGNTHTGFNDQGGPQKSVNFVVAHDGFTMMDLV |
| | | | SYDSKQNTALSWPFGPSDGGSNDNDSWNTNNDPVLRR |
| | | | QQLRNLWTILMFSRGVPMTVWGDEFARTQNGNNNPYNV |
| | | | DSICTYNNYNMIASDSPNTVSTGYAAAYHNNFGTDTNADN |
| | | | ANTIFKFARNVIQLRKNSSALKHSSYDMTYTYKKEDGVSD |
| | | | LQDDNRCVWIRIDGSSVAGGSDYLLLINTYTNLVNYTVPTA |
| | | | DAGKKWVRIIDTASWAETNDNYWSAASGATITGSYGVNP |
| | | | YSIVVLQEVAQ |
| OQB68755.1 | Spirochaetes | 277 | CSNHSSLKYDNEDQDFAYEKGYGLLEVNIPTVRGWTVAQ |
| (6X-His) | bacterium | | YDVVATKPGETSVTASTASTSVSLRLKIGTWTISVVGKDSY |
| | ADurb.Bin133 | | ANTIYQGVASANVTESGNSVTIGLLKKAGNYKLTLNSTYPV |
| | | | VSGQPGYIEKIVVTASRGQGFADVVAESNAFSNSFIFSGL |
| | | | AQGNWTFTARGYAAGLDSDYQPTGTEVLFLEDSYTLNVV |
| | | | ASKITSSTQNLNTQKKATPVKFSHLAGTYSGTINVSLSCDT |
| | | | AGATIYYTTDGSNPTTSGTKKTYSTSVAISATTTLKVAAVK |
| | | | SGLTSSIVGSRLYTIDAGVTSTPQMNPTGGTYNSDQNVVL |
| | | | SCADAGAIIYYTTDGSNPTTSSTQYSAPIAVSGNGTTKVIR |
| | | | AIAKAGAKNVSGAASQSYTISYLASSAPTFTPASGNLTTND |
| | | | NITIECGTSGASIYYTTDGSDPKISGTRTPYTSPFSLAVGTY |
| | | | TVKAYSTASGYADSTVTSGEFNVTQPQQDNSIIFATAETIIS |
| | | | GEEWNPSSLAAGINLFGVDIFDTGTFKLEWSETYSGSVTM |
| | | | YENDLTTPLSLTGTGPSRTVSLVSGERYFINFNSTYEMAN |
| | | | FSVRVYLGASGGGGALGSNYPSSGSYSPVNMADWGTAT |
| | | | WELGANYVSGPPSNLRIGVFAANATKVLLEIYSQKTGQTA |
| | | | QYDYWMAKGSDGVWRAELNSVPNYALYAFRAWGPNWS |
| | | | FDSNWTRGNSASGFTSDVDAAGNRYNPNKVLYDPYAKEI |
| | | | SHDKETPEMAAAGENGGMYGTGGLTDLTPHGYRGPCTN |
| | | | NVNIDIRNVDTGKWAPKSVAVYNTTSFGTKPNIAEKDGIIY |
| | | | EAHVRGLTQHSSSISLPSILSGVGINTSSWGTWFTDAERG |
| | | | TYKAAGKMAKYLKALGYTTIELLPVHETANDINPADSPGG |
| | | | NYWGYMTYGYFAPDRRYSYDKTFGGPTKEFKEMCAAFH |
| | | | AEGLEVYLDVVYNHTGEGGNWDPSHIESPVGSGNWIENP |
| | | | DFTTKCKEITSFAGLDNANYYALVSTDKGRYWETTGCGN |
| | | | NMDVSKAVVRDLIKDSLKYWITEMGVDGFRFDLAPVLGR |
| | | | DAAPNYHFNPNGQLLTEIAAMGATYNVEMIAEAWDCQWP |
| | | | GGYQVSNFPAGWGEWNGFYRDSVRKFIKGGGNKTSGYP |
| | | | SFTDAFNGSYGPLDRSSGNTHTGFNDQGGPQKSVNFVV |
| | | | AHDGFTMMDLVSYDSKQNTALSWPFGPSDGGSNDNDS |
| | | | WNTNNDPVLRRQQLRNLWTILMFSRGVPMTVWGDEFAR |
| | | | TQNGNNNPYNVDSICTYNNYNMIASDSPNTVSTGYAAAY |
| | | | HNNFGTDTNADNANTIFKFARNVIQLRKNSSALKHSSYDM |
| | | | TYTYKKEDGVSDLQDDNRCVWIRIDGSSVAGGSDYLLLIN |
| | | | TYTNLVNYTVPTADAGKKWVRIIDTASWAETNDNYWSAA |
| | | | SGATITGSYGVNPYSIVVLQEVAQHHHHHH |
| ADY25783.1 | Deinococcus | 278 | MTETASPTALTTGQPQPLGATREEGGTNFAVYAPDATRV |
| | proteolyticus | | ELCLLTEEGERCVDLPERTGNVWHGLLPDDHDYSRGVG |
| | (strain ATCC | | QGYGYRVHGHHDPENGVYAQPEVRLLDPYARALSGPEE |
| | 35074/DSM | | PIRNEEGHVASLQAPWGLVLDTEVEVPLEDKPQVPWNHT |
| | 20540/JCM | | VIYETHVRGLTMTHPGVPEELRGTYAGLACEPVVKYLKDL |
| | 6276/NBRC | | GITAVELLPVHAHVDDPFLQNKGLHNYWGYSTLSYFAPEP |
| | 101906/NCIMB | | RYSAAARAGRPQDTEQEFRDMVAKLHEAGLEVILDVVYN |
| | 13154/VKM Ac- | | HTAEGGKGGPLLSWRGLANSTYYWLSQDDLGEYFDFTG |
| | 1939/CCM 2703/ | | TGNSVRMTHRRTVQMVLDSMRHWAALGVDGFRFDLAST |
| | MRP) | | LARGELGFDKHSNFLGAVQADPVLNRLKLIAEPWDVGLG |
| | | | GYQVGNFPPPWAEWNDQYRDTVRGFWKGDEGLMSEM |
| | | | GFRLTGSSDIYSANHRHPQASINFITAHDGFTLRDVVSYN |
| | | | DKHNDANGEDNRDGHGNNLSWNMGAEGETDDAEVLRE |
| | | | RRKQQRNLLTTLLISQGIPMILGGDELGRTQGGNNNTYAQ |
| | | | DNEINWYDWQNADRDLLAFTRRLIGLRQDHPSFRRERFF |
| | | | SGRREEGELPHILWLRYDGQEMNDTDWQNPQTKSVGLF |
| | | | LYGGEEEGEVRDHLLVLLNASHVDLPFNLPSFREQGPEQ |
| | | | TCERWTLLLDTADDAAQEQVEADQDTQLTARSIKIFSCSA |
| | | | QQG |
| ADY25783.1 | Deinococcus | 279 | MTETASPTALTTGQPQPLGATREEGGTNFAVYAPDATRV |
| (6X-His) | proteolyticus | | ELCLLTEEGERCVDLPERTGNVWHGLLPDDHDYSRGVG |
| | (strain ATCC | | QGYGYRVHGHHDPENGVYAQPEVRLLDPYARALSGPEE |
| | 35074/DSM | | PIRNEEGHVASLQAPWGLVLDTEVEVPLEDKPQVPWNHT |
| | 20540/JCM | | VIYETHVRGLTMTHPGVPEELRGTYAGLACEPVVKYLKDL |
| | 6276/NBRC | | GITAVELLPVHAHVDDPFLQNKGLHNYWGYSTLSYFAPEP |
| | 101906/NCIMB | | RYSAAARAGRPQDTEQEFRDMVAKLHEAGLEVILDVVYN |
| | 13154/VKM Ac- | | HTAEGGKGGPLLSWRGLANSTYYWLSQDDLGEYFDFTG |
| | 1939/CCM 2703/ | | TGNSVRMTHRRTVQMVLDSMRHWAALGVDGFRFDLAST |
| | MRP) | | LARGELGFDKHSNFLGAVQADPVLNRLKLIAEPWDVGLG |
| | | | GYQVGNFPPPWAEWNDQYRDTVRGFWKGDEGLMSEM |
| | | | GFRLTGSSDIYSANHRHPQASINFITAHDGFTLRDVVSYN |
| | | | DKHNDANGEDNRDGHGNNLSWNMGAEGETDDAEVLRE |
| | | | RRKQQRNLLTTLLISQGIPMILGGDELGRTQGGNNNTYAQ |
| | | | DNEINWYDWQNADRDLLAFTRRLIGLRQDHPSFRRERFF |
| | | | SGRREEGELPHILWLRYDGQEMNDTDWQNPQTKSVGLF |
| | | | LYGGEEEGEVRDHLLVLLNASHVDLPFNLPSFREQGPEQ |
| | | | TCERWTLLLDTADDAAQEQVEADQDTQLTARSIKIFSCSA |
| | | | QQGHHHHHH |
| SOU43059.1 | Pseudoalteromonas | 280 | MMSNLFDAAHGNVEPLGSSVSDNGVNFSLYAPNASQAY |
| | carrageenovora | | VCLFDKSGHSEILKMAMNINEGGVWSIHISPLSAGALYGF |
| | IAM 12662 | | RVEGEYNPSHGLLFNENKLLIDPYAKDIFGEFTWSERHYG |
| | | | QMPIGTKSCVNNSIDIPKSKVAAQVHYEHKKPKHRWSNTV |
| | | | IYECHVKGTTCRHPKIPKSLQGKFLGLSHPSFIEHLHNLGV |
| | | | TAVELLPVHAFISEQFLTAKGLQNYWGYNTLNFFTPHKDY |
| | | | LVNDDINEFKQMVKQFHDADIEVILDVVYNHTAEAGNDGPI |
| | | | LSLRGLDNLAYYRTAHDQPNVYINDTGCGNTINIDHPKTLQ |
| | | | LVLDSLRYWVEVMGVDGFRFDLATILARSKSGFSSGHTFL |
| | | | QAIAQDPVLNKVKLISEPWDIGPGGYQLGAFPPPWREWN |
| | | | DQYRDVIRRFWQSETGIIANVAKRLHGSFDIFEHSQRGPL |
| | | | NSINFITSHDGFTLADLVSYEHKHNEANGEQNRDGHSANH |
| | | | SFNCGVEGFTSDVKITSLRLQQQKNFLLTLILSKGVPMIAA |
| | | | GSEMAHSQGGNNNAYCQNNRTSWLAWKDSQLNHSLTR |
| | | | FIDDALKIRRAHSAFKHSVFLDDIDERFTVKWFTEQGKTMT |
| | | | DAHWHEQTRQFLMYSLLDKQNKHALLIVFNASKKTIFCQL |
| | | | PPSPIKAQWQMVLSSVNNASARSNEDAMVEISAQSSWVF |
| | | | SANLEDVGHG |
| SOU43059.1 | Pseudoalteromonas | 281 | MMSNLFDAAHGNVEPLGSSVSDNGVNFSLYAPNASQAY |
| (6X-His) | carrageenovora | | VCLFDKSGHSEILKMAMNINEGGVWSIHISPLSAGALYGF |
| | IAM 12662 | | RVEGEYNPSHGLLFNENKLLIDPYAKDIFGEFTWSERHYG |
| | | | QMPIGTKSCVNNSIDIPKSKVAAQVHYEHKKPKHRWSNTV |
| | | | IYECHVKGTTCRHPKIPKSLQGKFLGLSHPSFIEHLHNLGV |
| | | | TAVELLPVHAFISEQFLTAKGLQNYWGYNTLNFFTPHKDY |
| | | | LVNDDINEFKQMVKQFHDADIEVILDVVYNHTAEAGNDGPI |
| | | | LSLRGLDNLAYYRTAHDQPNVYINDTGCGNTINIDHPKTLQ |
| | | | LVLDSLRYWVEVMGVDGFRFDLATILARSKSGFSSGHTFL |
| | | | QAIAQDPVLNKVKLISEPWDIGPGGYQLGAFPPPWREWN |
| | | | DQYRDVIRRFWQSETGIIANVAKRLHGSFDIFEHSQRGPL |
| | | | NSINFITSHDGFTLADLVSYEHKHNEANGEQNRDGHSANH |
| | | | SFNCGVEGFTSDVKITSLRLQQQKNFLLTLILSKGVPMIAA |
| | | | GSEMAHSQGGNNNAYCQNNRTSWLAWKDSQLNHSLTR |
| | | | FIDDALKIRRAHSAFKHSVFLDDIDERFTVKWFTEQGKTMT |
| | | | DAHWHEQTRQFLMYSLLDKQNKHALLIVFNASKKTIFCQL |
| | | | PPSPIKAQWQMVLSSVNNASARSNEDAMVEISAQSSWVF |
| | | | SANLEDVGHGHHHHHH |
| ADZ92347.1 | Marinomonas | 282 | MPLAPLVRSGSPLPLGANVTDEGVNFAVASEDATQIYLCL |
| | mediterranea | | FNENDEEEHVIPFVSHHRGIWHMEVLGLKEGQHYGFRAE |
| | (strain ATCC | | GTFSPKDQLMFNRHKLLIDPYAKSLTGTLEWHPDLSAMYS |
| | 700492/JCM | | DGHFNPIDTAHYVPKSIVKTITRPDDAPARVSVKGAERSLY |
| | 21426/NBRC | | ELHIKGFSKNLEVSPELKGTYLGVVSEQGISHLKELGVTTI |
| | 103028/MMB-1) | | QLMPCFAFANERHLQKLGLQNYWGYNPINFFTPDHRYAV |
| | | | EDPVIECQTMIMSLKRAGFEVIIDVVYNHTAESELDQASFS |
| | | | FKGLDNARYYRHENGEYLNYTGCGNCVNTYHPAAVRLIC |
| | | | DSMRYWVDVMGVDGFRFDLGVDLGREKHDFSSTSPLLQ |
| | | | AILQDPVLSETCLVMEPWDIGPNGYQVGNFPRGFLECND |
| | | | KYRDIVRRFWRGDEGIVGEFATRFMGSRDVFHKGYKCAL |
| | | | HSVNYISYHDGYTLHDLVSYHRRHNHANMENNRDGHGD |
| | | | NISQNFGVEGPTSDNRIKAQRLNQQLCFLATLLLSQGTPHI |
| | | | LGGDELSHTQHGNNNAYCQDNQTTWLGWQNSDEREQL |
| | | | QSAIKTLLNLRKAYPVLAEVFLEDDPLYQYKESDKVEWVN |
| | | | EAGRPMSNEDWSESERDFLSVLFTTSDLSSRLWLIFYRE |
| | | | DQVLDVPLIKNARQHQLLFYTPGVAMRDNHLHITERSVAL |
| | | | VKLLP |
| ADZ92347.1 | Marinomonas | 283 | MPLAPLVRSGSPLPLGANVTDEGVNFAVASEDATQIYLCL |
| (6X-His) | mediterranea | | FNENDEEEHVIPFVSHHRGIWHMEVLGLKEGQHYGFRAE |
| | (strain ATCC | | GTFSPKDQLMFNRHKLLIDPYAKSLTGTLEWHPDLSAMYS |
| | 700492/JCM | | DGHFNPIDTAHYVPKSIVKTITRPDDAPARVSVKGAERSLY |
| | 21426/NBRC | | ELHIKGFSKNLEVSPELKGTYLGVVSEQGISHLKELGVTTI |
| | 103028/MMB-1) | | QLMPCFAFANERHLQKLGLQNYWGYNPINFFTPDHRYAV |
| | | | EDPVIECQTMIMSLKRAGFEVIIDVVYNHTAESELDQASFS |
| | | | FKGLDNARYYRHENGEYLNYTGCGNCVNTYHPAAVRLIC |
| | | | DSMRYWVDVMGVDGFRFDLGVDLGREKHDFSSTSPLLQ |
| | | | AILQDPVLSETCLVMEPWDIGPNGYQVGNFPRGFLECND |
| | | | KYRDIVRRFWRGDEGIVGEFATRFMGSRDVFHKGYKCAL |
| | | | HSVNYISYHDGYTLHDLVSYHRRHNHANMENNRDGHGD |
| | | | NISQNFGVEGPTSDNRIKAQRLNQQLCFLATLLLSQGTPHI |
| | | | LGGDELSHTQHGNNNAYCQDNQTTWLGWQNSDEREQL |
| | | | QSAIKTLLNLRKAYPVLAEVFLEDDPLYQYKESDKVEWVN |
| | | | EAGRPMSNEDWSESERDFLSVLFTTSDLSSRLWLIFYRE |
| | | | DQVLDVPLIKNARQHQLLFYTPGVAMRDNHLHITERSVAL |
| | | | VKLLPHHHHHH |
| AEG44960.1 | Isoptericola | 284 | MDARPKVTPLTRPTARRSHVSPLGVHVTPDGGVDVAVVA |
| | variabilis | | SHATGVDLCLIDVVDPSRDERDPARYRERRVPLDGPTYG |
| | (strain 225) | | VWHAHVPGVAPGQRYGFRAHGPWDPAAGHRHNPAKLL |
| | | | VDPYARGLVGEVAYGPETLGSPGRPRSGDPSTPDGRWW |
| | | | LADPYGEPDEVDSIAHVPHAVVMPALPGPAPLSRPRVPW |
| | | | SDTVIYEAHVRGLTQLNPDVPEELRGTYAGVAHPATIAHL |
| | | | QALGVTTLELLPIHASVSEPRLVAQGLTNYWGYQTLGFFA |
| | | | PHARYATKGAQEAGPAAVLDEVRGMVHLLHEAGIEVILDV |
| | | | VYNHTCEGGEDSLHLSWRGLDNAMYYVHDGGTPARLAD |
| | | | VTGTGNSLDFRRVRVVQMALDSLRYWAEVVGVDGFRFD |
| | | | LAVTLARGHYGFDPDHPFLVGLQTDPVLSGLKLVAEPWD |
| | | | VGPRGWRTGQFPPPVAEWNDKFRDSVRAFWLDAPRQG |
| | | | SRGQPMHGMRELATRLAGSADLFGHSDPPLVRGPVASIN |
| | | | FVTAHDGFTLADLVAFDHKHNWANGEDNRDGHGHNLSW |
| | | | NHGVEGHAPEGDDASTEPWSTVVPLRRRSQRNLLAMTLL |
| | | | AAGTPMLTAGDEIGRSQRGNNNAYALDDETSWLGWDVD |
| | | | DAARHLLETARFLVGLRRRHAALRAESFFLGKPRPGETLP |
| | | | DLLWFDAGGSPMDHDAWNVAGRRVIQMLRTGPGAGESD |
| | | | VLLVLNGGLDDVEVHLPQPLDGAGAWERVWSSTWDTPE |
| | | | EPEEEPLTCPVTLEALSVEVFVAPR |
| AEG44960.1 | Isoptericola | 285 | MDARPKVTPLTRPTARRSHVSPLGVHVTPDGGVDVAVVA |
| (6X-His) | variabilis | | SHATGVDLCLIDVVDPSRDERDPARYRERRVPLDGPTYG |
| | (strain 225) | | VWHAHVPGVAPGQRYGFRAHGPWDPAAGHRHNPAKLL |
| | | | VDPYARGLVGEVAYGPETLGSPGRPRSGDPSTPDGRWW |
| | | | LADPYGEPDEVDSIAHVPHAVVMPALPGPAPLSRPRVPW |
| | | | SDTVIYEAHVRGLTQLNPDVPEELRGTYAGVAHPATIAHL |
| | | | QALGVTTLELLPIHASVSEPRLVAQGLTNYWGYQTLGFFA |
| | | | PHARYATKGAQEAGPAAVLDEVRGMVHLLHEAGIEVILDV |
| | | | VYNHTCEGGEDSLHLSWRGLDNAMYYVHDGGTPARLAD |
| | | | VTGTGNSLDFRRVRVVQMALDSLRYWAEVVGVDGFRFD |
| | | | LAVTLARGHYGFDPDHPFLVGLQTDPVLSGLKLVAEPWD |
| | | | VGPRGWRTGQFPPPVAEWNDKFRDSVRAFWLDAPRQG |
| | | | SRGQPMHGMRELATRLAGSADLFGHSDPPLVRGPVASIN |
| | | | FVTAHDGFTLADLVAFDHKHNWANGEDNRDGHGHNLSW |
| | | | NHGVEGHAPEGDDASTEPWSTVVPLRRRSQRNLLAMTLL |
| | | | AAGTPMLTAGDEIGRSQRGNNNAYALDDETSWLGWDVD |
| | | | DAARHLLETARFLVGLRRRHAALRAESFFLGKPRPGETLP |
| | | | DLLWFDAGGSPMDHDAWNVAGRRVIQMLRTGPGAGESD |
| | | | VLLVLNGGLDDVEVHLPQPLDGAGAWERVWSSTWDTPE |
| | | | EPEEEPLTCPVTLEALSVEVFVAPRHHHHHH |
| CAC49675.1 | Rhizobium | 286 | MNLTFSELDFMKPELGAEYTGQGTHFAVFSAHAEKIELCL |
| | meliloti (strain | | FSEDGREETARMPLPKREGDIWSGYIEGIGPGTLYGYRAH |
| | 1021) | | GPYDPHNGHRFNPNKLLLDPYAKLVAGDLIWDDALFGYTI |
| | | | GSPEGDLSFDERDSAPFMVKGVVQDPTFDWAGEEAIRRP |
| | | | WTETIIYESHVRGMTMTHPGVPDELRGTFLGMASDPIIEHL |
| | | | TKLGVSAIELMPVQYFLDDRYLVERNLRNYWGYQTLGFFA |
| | | | PHDRYLKGDRFTEFKTMVKRFHSVGIEVLMDVVYNHTAE |
| | | | GSERGPTLSFRGLDNFSYYRQSPDKPRHTYDTTGTGNTL |
| | | | NVAHPLVLRMVLDSLRYWVGVMHIDGFRFDLASTLGREY |
| | | | MEFDREGGFFDAIRQDPILAGVKLIAEPWDVGEGGYQLG |
| | | | GFPPPFREWNDRFRDDVRRFWKGDGGMVPVLAERILGS |
| | | | PVQFRHSDRAATSSINLVSAHDGFTLMDTVSYGEKHNEA |
| | | | NGEENRDGHSDNHSDNMGAEGPTDNREIIEARKRRRAA |
| | | | MLATLMISQGVPMVLGGDELGNSQQGNNNAYCQDNEIG |
| | | | WLDWSGADDGFVDFCRKLLAFRKDRPVLRQERFLDGEP |
| | | | DENGRVEIAWYKADGSPMDEDAWHDAELRLICAYICRAG |
| | | | QGEAPPAGEIVLVLNAGGDCEIALPHGNGSRRWLRVLDT |
| | | | AADEPFGMRASADRDTIPAQSVVAFVPQEGSNG |
| CAC49675.1 | Rhizobium | 287 | MNLTFSELDFMKPELGAEYTGQGTHFAVFSAHAEKIELCL |
| (6X-His) | meliloti (strain | | FSEDGREETARMPLPKREGDIWSGYIEGIGPGTLYGYRAH |
| | 1021) | | GPYDPHNGHRFNPNKLLLDPYAKLVAGDLIWDDALFGYTI |
| | | | GSPEGDLSFDERDSAPFMVKGVVQDPTFDWAGEEAIRRP |
| | | | WTETIIYESHVRGMTMTHPGVPDELRGTFLGMASDPIIEHL |
| | | | TKLGVSAIELMPVQYFLDDRYLVERNLRNYWGYQTLGFFA |
| | | | PHDRYLKGDRFTEFKTMVKRFHSVGIEVLMDVVYNHTAE |
| | | | GSERGPTLSFRGLDNFSYYRQSPDKPRHTYDTTGTGNTL |
| | | | NVAHPLVLRMVLDSLRYWVGVMHIDGFRFDLASTLGREY |
| | | | MEFDREGGFFDAIRQDPILAGVKLIAEPWDVGEGGYQLG |
| | | | GFPPPFREWNDRFRDDVRRFWKGDGGMVPVLAERILGS |
| | | | PVQFRHSDRAATSSINLVSAHDGFTLMDTVSYGEKHNEA |
| | | | NGEENRDGHSDNHSDNMGAEGPTDNREIIEARKRRRAA |
| | | | MLATLMISQGVPMVLGGDELGNSQQGNNNAYCQDNEIG |
| | | | WLDWSGADDGFVDFCRKLLAFRKDRPVLRQERFLDGEP |
| | | | DENGRVEIAWYKADGSPMDEDAWHDAELRLICAYICRAG |
| | | | QGEAPPAGEIVLVLNAGGDCEIALPHGNGSRRWLRVLDT |
| | | | AADEPFGMRASADRDTIPAQSVVAFVPQEGSNGHHHHH |
| | | | H |
| ABG98225.1 | Rhodococcus | 288 | MSPHIDSHPGKMSSLPPSLRLPGSPFPLGAKVRGTGTQF |
| | jostii (strain | | AVHAPDAELVQVCLISGDGDEMRIDLVQRTYGIWHGIVEG |
| | RHA1) | | VGAGQRYGYRTHGRWDPARGVRLNPAKLLLDPWATRIT |
| | | | GKLGDHDALVSHDGDPFGGPSRVDSLGHTPLSVVTADHA |
| | | | RTSGPRIERPWEETVVYELHVGSFTARHPDVPPQYRGTY |
| | | | LGLAEPAVVGHLVRLGVTAVELLPVQAYVSEATVRSRGM |
| | | | RNHWGYSTAAFFAPHPGYAVVPGEEITEFHTMVDALHAA |
| | | | GIEVILDVVYNHTCEQGVDGISLSWRGLDAPGYYLLDSDG |
| | | | RDIDITGCGNTVDAQSPVVVRMIGDSLRYWAHEMGVDGF |
| | | | RFDLASALGRPGGGRFDSRASIFSAITADPVLSRCKLIAEP |
| | | | WDATSDGYRVGHFGVQWSEWNDKYRNTVRRFWNGGT |
| | | | GVRDLASRVAGSEDLFGNRRPWASVNFVTAHDGFTAADL |
| | | | VSYVRKHNEANGEDNRDGADHNDSVDHGVEGPTDDPSII |
| | | | ETRSRHVRALLATLALSTGTPMFTAGDEFGHTLGGNNNA |
| | | | YCVPEDTPREDSWPLDWSTGDTTLTAFVTRALDLRRASP |
| | | | ALRQPEFFEGRRTPTGHPDLVWFGADGAEMDDPAWYDD |
| | | | SRRTLQAWIDGSDIRSHTEDGRQLTDDSWLLVFHSGGPA |
| | | | VITLGCPEWFYGTVLAEFDSTSPDGAPAPGGPISGDSTIAL |
| | | | SGPTLLVFRAVG |
| ABG98225.1 | Rhodococcus | 289 | MSPHIDSHPGKMSSLPPSLRLPGSPFPLGAKVRGTGTQF |
| (6X-His) | jostii (strain | | AVHAPDAELVQVCLISGDGDEMRIDLVQRTYGIWHGIVEG |
| | RHA1) | | VGAGQRYGYRTHGRWDPARGVRLNPAKLLLDPWATRIT |
| | | | GKLGDHDALVSHDGDPFGGPSRVDSLGHTPLSVVTADHA |
| | | | RTSGPRIERPWEETVVYELHVGSFTARHPDVPPQYRGTY |
| | | | LGLAEPAVVGHLVRLGVTAVELLPVQAYVSEATVRSRGM |
| | | | RNHWGYSTAAFFAPHPGYAVVPGEEITEFHTMVDALHAA |
| | | | GIEVILDVVYNHTCEQGVDGISLSWRGLDAPGYYLLDSDG |
| | | | RDIDITGCGNTVDAQSPVVVRMIGDSLRYWAHEMGVDGF |
| | | | RFDLASALGRPGGGRFDSRASIFSAITADPVLSRCKLIAEP |
| | | | WDATSDGYRVGHFGVQWSEWNDKYRNTVRRFWNGGT |
| | | | GVRDLASRVAGSEDLFGNRRPWASVNFVTAHDGFTAADL |
| | | | VSYVRKHNEANGEDNRDGADHNDSVDHGVEGPTDDPSII |
| | | | ETRSRHVRALLATLALSTGTPMFTAGDEFGHTLGGNNNA |
| | | | YCVPEDTPREDSWPLDWSTGDTTLTAFVTRALDLRRASP |
| | | | ALRQPEFFEGRRTPTGHPDLVWFGADGAEMDDPAWYDD |
| | | | SRRTLQAWIDGSDIRSHTEDGRQLTDDSWLLVFHSGGPA |
| | | | VITLGCPEWFYGTVLAEFDSTSPDGAPAPGGPISGDSTIAL |
| | | | SGPTLLVFRAVGHHHHHH |
| AEN72910.1 | Rhodothermus | 290 | MSHSAQPVTSVQAVWPGRPYPLGATWDGLGVNFALYSQ |
| | marinus (strain | | HAEAVELVLFDHPDDPAPSRTIEVTERTGPIWHVYLPGLR |
| | SG0.5JP17-172) | | PGQLYGYRVYGPYRPEESHRFNPNKVLLDPYAKAIGRPL |
| | | | RWHDSLFGYKIGDPAGDLSFSEEDSAPYAPLGAVVEGCF |
| | | | EWGDDRPPRIPWEDTIIYETHVKGITKLHPEVPEPLRGTYL |
| | | | GLTCEPVLEHLKRLGVTTIQLLPVHAKVHDRHLVERGLRN |
| | | | YWGYNPLCYFAPEPEYATNGPISAVREFKMMVRALHAAG |
| | | | FEVIVDVVYNHTGEGGVLGPTLSFRGIDNRAYYKADPNNP |
| | | | RFLVDYTGTGNTLDVGNPYVIQLIMDSLRYWVTEMHVDG |
| | | | FRFDLAAALARELYDVDMLSTFFQVIQQDPVLSQVKLIAEP |
| | | | WDVGPGGYQVGHFPWQWTEWNGRYRDAVRRFWRGDR |
| | | | GLNGEFATRFAGSSDLYERSGRRPFASINFVTAHDGFTLE |
| | | | DLVSYTKKHNEANLEGNRDGMDENYSTNCGVEGPTQDP |
| | | | SVLACREALKRSLISTLFLSQGVPMLLGGDELSRTQHGNN |
| | | | NAYCQDNEISWYNWQLDTRKQQFLEFVRQVIWFRKQHR |
| | | | SFRRRHFLTGLPNGGEAPDAVWWHPEGRPMRHEDWTN |
| | | | PELTAFGLLLHGDAIQGTDEHGRPFRDDTFLILFNNGSEAV |
| | | | PVVVPEVCSCGKPHHWEVVPVFQRNVEPPTCAPGETLSL |
| | | | PPGVLTVLVAVPPFSDGNPETA |
| AEN72910.1 | Rhodothermus | 291 | MSHSAQPVTSVQAVWPGRPYPLGATWDGLGVNFALYSQ |
| (6X-His) | marinus (strain | | HAEAVELVLFDHPDDPAPSRTIEVTERTGPIWHVYLPGLR |
| | SG0.5JP17-172) | | PGQLYGYRVYGPYRPEESHRFNPNKVLLDPYAKAIGRPL |
| | | | RWHDSLFGYKIGDPAGDLSFSEEDSAPYAPLGAVVEGCF |
| | | | EWGDDRPPRIPWEDTIIYETHVKGITKLHPEVPEPLRGTYL |
| | | | GLTCEPVLEHLKRLGVTTIQLLPVHAKVHDRHLVERGLRN |
| | | | YWGYNPLCYFAPEPEYATNGPISAVREFKMMVRALHAAG |
| | | | FEVIVDVVYNHTGEGGVLGPTLSFRGIDNRAYYKADPNNP |
| | | | RFLVDYTGTGNTLDVGNPYVIQLIMDSLRYWVTEMHVDG |
| | | | FRFDLAAALARELYDVDMLSTFFQVIQQDPVLSQVKLIAEP |
| | | | WDVGPGGYQVGHFPWQWTEWNGRYRDAVRRFWRGDR |
| | | | GLNGEFATRFAGSSDLYERSGRRPFASINFVTAHDGFTLE |
| | | | DLVSYTKKHNEANLEGNRDGMDENYSTNCGVEGPTQDP |
| | | | SVLACREALKRSLISTLFLSQGVPMLLGGDELSRTQHGNN |
| | | | NAYCQDNEISWYNWQLDTRKQQFLEFVRQVIWFRKQHR |
| | | | SFRRRHFLTGLPNGGEAPDAVWWHPEGRPMRHEDWTN |
| | | | PELTAFGLLLHGDAIQGTDEHGRPFRDDTFLILFNNGSEAV |
| | | | PVVVPEVCSCGKPHHWEVVPVFQRNVEPPTCAPGETLSL |
| | | | PPGVLTVLVAVPPFSDGNPETAHHHHHH |
| EHP91876.1 | Methylorubrum | 292 | MIVIEEGLPTPLGAHFDGRGVNFALFSQNATRVDLCLFDP |
| | extorquens DSM | | GARHESRTIRLPCRTDDVFHGYLHGLLPGQQYAYRVFGP |
| | 13060 | | WDPAAGHRFNPAKLVLDPYAREIAGRIRWHDALYSHRHG |
| | | | GAREDQIDRRDSAPFVPRGVVTRPDTPDAIALPAPRPLHE |
| | | | TVIYEAHVKALTRTHPALSEAERGTYLGLAHPAIIEHLLKLG |
| | | | VTALELLPIQAFADDRFLIDKGLVNFWGYQPYNYFAPEPR |
| | | | YLGEGGASGLRFAIRELASAGIETLIDVVYNHTAEGDHRG |
| | | | PTLAFRGIDNASYYKLDPDNLRRNIDCTGCGNTLNVAHPR |
| | | | VMRMVLDSLRHWVTAYGVAGFRFDLATSLGRAPSDFSP |
| | | | QAAFFQAVQQDPVLSRVKLIAEPWDIGAGGYQLGGYPYG |
| | | | WSEWNDQFRDNLRGFWRGDSGTLAKLTQGLSGSREIFL |
| | | | PSGRSPLASINFVASHDGYTLADVVAYEEKHNEANGEEN |
| | | | RDGHGHNLSANYGVEGPTDDPALLALRARQKRNMLACV |
| | | | FFAQGVPMLLMGDERSRTQSGNNNAYCQDGALSWMDW |
| | | | ENDPDPTLTEFVANLAALRRVCCSLRRRHFLVGSRVGET |
| | | | ALKDVHWLSPDGTEMDAAAWGDGERRAFGMQMSNDIE |
| | | | DSERVLILMNAAPEPCPFALPPDLGGPWRPVFDTTLDTGK |
| | | | VLDGARPPVPVGGTVDLPERAVLVLKSPPLLD |
| EHP91876.1 | Methylorubrum | 293 | MIVIEEGLPTPLGAHFDGRGVNFALFSQNATRVDLCLFDP |
| (6X-His) | extorquens DSM | | GARHESRTIRLPCRTDDVFHGYLHGLLPGQQYAYRVFGP |
| | 13060 | | WDPAAGHRFNPAKLVLDPYAREIAGRIRWHDALYSHRHG |
| | | | GAREDQIDRRDSAPFVPRGVVTRPDTPDAIALPAPRPLHE |
| | | | TVIYEAHVKALTRTHPALSEAERGTYLGLAHPAIIEHLLKLG |
| | | | VTALELLPIQAFADDRFLIDKGLVNFWGYQPYNYFAPEPR |
| | | | YLGEGGASGLRFAIRELASAGIETLIDVVYNHTAEGDHRG |
| | | | PTLAFRGIDNASYYKLDPDNLRRNIDCTGCGNTLNVAHPR |
| | | | VMRMVLDSLRHWVTAYGVAGFRFDLATSLGRAPSDFSP |
| | | | QAAFFQAVQQDPVLSRVKLIAEPWDIGAGGYQLGGYPYG |
| | | | WSEWNDQFRDNLRGFWRGDSGTLAKLTQGLSGSREIFL |
| | | | PSGRSPLASINFVASHDGYTLADVVAYEEKHNEANGEEN |
| | | | RDGHGHNLSANYGVEGPTDDPALLALRARQKRNMLACV |
| | | | FFAQGVPMLLMGDERSRTQSGNNNAYCQDGALSWMDW |
| | | | ENDPDPTLTEFVANLAALRRVCCSLRRRHFLVGSRVGET |
| | | | ALKDVHWLSPDGTEMDAAAWGDGERRAFGMQMSNDIE |
| | | | DSERVLILMNAAPEPCPFALPPDLGGPWRPVFDTTLDTGK |
| | | | VLDGARPPVPVGGTVDLPERAVLVLKSPPLLDHHHHHH |
| CCG07661.1 | Pararho- | 294 | MMRRLVLEPSGPGPAGAWVGDGGVHWRVFSDNASAVE |
| | dospirillum | | VCLFDAEGREERLPLPGRDGSFFHGFLPGVGAGLRYGLR |
| | photometricum | | AHGPYDPARGHRFNPHKLLIDPLARHLDGPVIPHPALFGF |
| | DSM 122 | | DVARGWPQGEDDQPSGLDSAPFVPKAIVVDPAAPSPRPR |
| | | | LGGRLEPGRLVIYEAHVKGMTQQHPEVETVARGTFAGFA |
| | | | APGVVDHLRRLGVTAVELLPVWAFADEPALWGSGRTNY |
| | | | WGYNPIAFAAPHPAYGPPDAFCRMVEALHDAGLAVVLDV |
| | | | VINHTAEGGARGPTLSWRGLDNLSWYRLVEGNLAAYVDH |
| | | | TACGNSVQTEHPGVLAQIVNALRAWVVERGVDGFRFDLA |
| | | | VTPARNQGAFDPEGPFLRALAADPVLSSCLLIAEPWDAGP |
| | | | AGHALGRFPAPWLEWNDQARDAVRRFWLRGGSAGAFA |
| | | | TALAGSSPVFPAERGPLAGVTYVTCHDGFALADLVAYDTK |
| | | | HNLANGEDNRDGMDTNHSVNHGVEGPTGEPGILALREQ |
| | | | QRRNLILSLVLAQGVPMLRAGDELGQTHHGNNNPYCHDS |
| | | | PLTWLDWAATDPTFLAFVRQALALRQPGRCAFLTGQPGP |
| | | | DGWADVAWRRPDGHPMAADDWDDRAFALRLGAARLILV |
| | | | NGRATACSFVLPDDAAWSVVLASAPVDLPALPPLSVAVLA |
| | | | PDLAGE |
| CCG07661.1 | Pararho- | 295 | MMRRLVLEPSGPGPAGAWVGDGGVHWRVFSDNASAVE |
| (6X-His) | dospirillum | | VCLFDAEGREERLPLPGRDGSFFHGFLPGVGAGLRYGLR |
| | photometricum | | AHGPYDPARGHRFNPHKLLIDPLARHLDGPVIPHPALFGF |
| | DSM 122 | | DVARGWPQGEDDQPSGLDSAPFVPKAIVVDPAAPSPRPR |
| | | | LGGRLEPGRLVIYEAHVKGMTQQHPEVETVARGTFAGFA |
| | | | APGVVDHLRRLGVTAVELLPVWAFADEPALWGSGRTNY |
| | | | WGYNPIAFAAPHPAYGPPDAFCRMVEALHDAGLAVVLDV |
| | | | VINHTAEGGARGPTLSWRGLDNLSWYRLVEGNLAAYVDH |
| | | | TACGNSVQTEHPGVLAQIVNALRAWVVERGVDGFRFDLA |
| | | | VTPARNQGAFDPEGPFLRALAADPVLSSCLLIAEPWDAGP |
| | | | AGHALGRFPAPWLEWNDQARDAVRRFWLRGGSAGAFA |
| | | | TALAGSSPVFPAERGPLAGVTYVTCHDGFALADLVAYDTK |
| | | | HNLANGEDNRDGMDTNHSVNHGVEGPTGEPGILALREQ |
| | | | QRRNLILSLVLAQGVPMLRAGDELGQTHHGNNNPYCHDS |
| | | | PLTWLDWAATDPTFLAFVRQALALRQPGRCAFLTGQPGP |
| | | | DGWADVAWRRPDGHPMAADDWDDRAFALRLGAARLILV |
| | | | NGRATACSFVLPDDAAWSVVLASAPVDLPALPPLSVAVLA |
| | | | PDLAGEHHHHHH |
| AEG05574.1 | Sinorhizobium | 296 | MPTTGIPPLGVTRTPDGTRFTVWSHNAARIDLCLFDEAGN |
| | meliloti (strain | | TELSRLPMQRDGDVHSIALPDIAAGTRYGLRAEGVYSPDH |
| | BL225C) | | GLWFDPSKLLVDPYAAQLDRPFRHDQRLTVFGEETADLV |
| | | | PKAILTEVKPLEPRPPLFQAGGLIYEIAVKPFTILHPGVPER |
| | | | KRGTVAALAEPVIIDHLTSLGVSAVELMPVVAWIDERHLPP |
| | | | LGLHNGWGYNPIAPMALDPRLVPGGIEELRRTVEVLHQA |
| | | | GIGVVLDLVFNHSGESDRFGATLSMRGLDNLTYYRHAAG |
| | | | RPGELINDTGCGNTIACDHPVVQRLILDSLRHFVLAAGVD |
| | | | GFRFDLASILGRDMGGFRRDAALFSAISADPILCGRVLIAE |
| | | | PWDTGPGGYQLGNFPHSFLEWNDRARDDIRRYWRGDR |
| | | | HAIGVLATALAGSSDSFSRWGETATRSVNFIAAHDGFTLM |
| | | | DLVSYARKHNEANGEGNRDGHDENFSWNNGAEGATGD |
| | | | PEIAAFRQRDVMALLGTLFTSRGAIMLTAGDEGGRSQGG |
| | | | NNNAYAQDNAVTWLDWSKLDGKLVEHTARLSAMRRRFS |
| | | | VFGDTGFFTGNGDVAWLRLDGEPLTVEDWEHPATDNLV |
| | | | VMLATEDRRQKRPTRLAVVINRSHAPHPFRLPLSLEGEW |
| | | | RDALSDLTVPSFAPARSVTFLVEVFESISRKRI |
| AEG05574.1 | Sinorhizobium | 297 | MPTTGIPPLGVTRTPDGTRFTVWSHNAARIDLCLFDEAGN |
| (6X-His) | meliloti (strain | | TELSRLPMQRDGDVHSIALPDIAAGTRYGLRAEGVYSPDH |
| | BL225C) | | GLWFDPSKLLVDPYAAQLDRPFRHDQRLTVFGEETADLV |
| | | | PKAILTEVKPLEPRPPLFQAGGLIYEIAVKPFTILHPGVPER |
| | | | KRGTVAALAEPVIIDHLTSLGVSAVELMPVVAWIDERHLPP |
| | | | LGLHNGWGYNPIAPMALDPRLVPGGIEELRRTVEVLHQA |
| | | | GIGVVLDLVFNHSGESDRFGATLSMRGLDNLTYYRHAAG |
| | | | RPGELINDTGCGNTIACDHPVVQRLILDSLRHFVLAAGVD |
| | | | GFRFDLASILGRDMGGFRRDAALFSAISADPILCGRVLIAE |
| | | | PWDTGPGGYQLGNFPHSFLEWNDRARDDIRRYWRGDR |
| | | | HAIGVLATALAGSSDSFSRWGETATRSVNFIAAHDGFTLM |
| | | | DLVSYARKHNEANGEGNRDGHDENFSWNNGAEGATGD |
| | | | PEIAAFRQRDVMALLGTLFTSRGAIMLTAGDEGGRSQGG |
| | | | NNNAYAQDNAVTWLDWSKLDGKLVEHTARLSAMRRRFS |
| | | | VFGDTGFFTGNGDVAWLRLDGEPLTVEDWEHPATDNLV |
| | | | VMLATEDRRQKRPTRLAVVINRSHAPHPFRLPLSLEGEW |
| | | | RDALSDLTVPSFAPARSVTFLVEVFESISRKRIHHHHHH |
| EEH13463.1 | Brucella ceti | 298 | MAATQACGRCEMSGLTVSIRGRNGAFAIEAGFAAEGGVT |
| | str. Cudo | | ALFGHSGAGKTTLLKMIAGTLRPENGRIAVGDFTLFDAQK |
| | | | GINLPPEKRRIGYVFQDARLFAYMSVKRNLTYARWAGHR |
| | | | QATRSFDEVVALLGIGHLLDRRPSTLSGGERQRVAIGRAL |
| | | | LSDPALLLLDEPLSSLDHARRQEILPFIERLRDESHVPIVYV |
| | | | SHEIDEVARLADQIVLLSAGRVTASGAAADIFPLIDAESEG |
| | | | GGVLLEGIVSAYDERYKLAEIDLGGASFQLSDAGLKQTMH |
| | | | VRLRVRARDVSIARKIPEAISIRNLLPVTVTGIERGEGPNAH |
| | | | VFLDFRGRRLGARLTRRSVDDLGLSVGDQVVALVKAVSV |
| | | | DRAAIREK |
| EEH13463.1 | Brucella ceti | 299 | MAATQACGRCEMSGLTVSIRGRNGAFAIEAGFAAEGGVT |
| (6X-His) | str. Cudo | | ALFGHSGAGKTTLLKMIAGTLRPENGRIAVGDFTLFDAQK |
| | | | GINLPPEKRRIGYVFQDARLFAYMSVKRNLTYARWAGHR |
| | | | QATRSFDEVVALLGIGHLLDRRPSTLSGGERQRVAIGRAL |
| | | | LSDPALLLLDEPLSSLDHARRQEILPFIERLRDESHVPIVYV |
| | | | SHEIDEVARLADQIVLLSAGRVTASGAAADIFPLIDAESEG |
| | | | GGVLLEGIVSAYDERYKLAEIDLGGASFQLSDAGLKQTMH |
| | | | VRLRVRARDVSIARKIPEAISIRNLLPVTVTGIERGEGPNAH |
| | | | VFLDFRGRRLGARLTRRSVDDLGLSVGDQVVALVKAVSV |
| | | | DRAAIREKHHHHHH |
| BAD | Geobacillus | 300 | MLHISRTFAAYLDEMDQIVVLAPKSLGFDGMAPFTLVAPS |
| 77112.10 | kaustophilus | | GEEIPLSVQHVEDVGETVKYVCRFASAFEFGATYWVRSC |
| | (strain HTA426) | | RGEETDVQIGAVVRTPAFDDRFFYDGPLGAEYLKEQTVF |
| | | | RVWAPTATAVSVKLVHPHLDEIRCVPLVRGERGVWSAVV |
| | | | PGDWERARYTYIACINRVWREAVDPYATAVSVNGEFGVVI |
| | | | DWEKTKLAPPSLPLPPLCSPTDAIIYELSIRDFTSHPDSGA |
| | | | VHKGKYLGLAETNTSGPNGTATGLSYVKELGVTHVQLMP |
| | | | FMDFAGVDERDPQAAYNWGYNPLHLYAPEGSYATDPAD |
| | | | PYARIVELKQAIHTLHENGLRVVMDAVYNHVYDREQSPLE |
| | | | KLVPGYYFRYDAYGQPANGTGVGNDIASERRMARRWIVD |
| | | | SVVFWAKEYGIDGFRFDLMGVHDIETMKAVRDALDAIDPS |
| | | | ILVYGEGWDLPTPLPPEQKATMANAKQLPRFAYFNDRFR |
| | | | DAVKGSTFHLPDRGFALGNPGGREQVKLAIAGSLRALGG |
| | | | LFCHPRQSINYVECHDNHTFWDKMEAANHDEPEWLRRK |
| | | | RQKLATAIVLLAQGIPFLHSGQEFYRTKGGDGNSYRSPDA |
| | | | VNQLDWERKSRYEDDVRYVQGLIALRRAHGAFRLATEAE |
| | | | VLRHFTFLEPLPPSVIAYRLHDAAVYGPWEDIIVVHHNEEK |
| | | | ETAIALPDEREWAVVCDGQRCGTTPFGQARGMLRLDGIG |
| | | | TWVLVHPAG |
| BAD | Geobacillus | 301 | MLHISRTFAAYLDEMDQIVVLAPKSLGFDGMAPFTLVAPS |
| 77112.10 | kaustophilus | | GEEIPLSVQHVEDVGETVKYVCRFASAFEFGATYWVRSC |
| (6X-His) | (strain HTA426) | | RGEETDVQIGAVVRTPAFDDRFFYDGPLGAEYLKEQTVF |
| | | | RVWAPTATAVSVKLVHPHLDEIRCVPLVRGERGVWSAVV |
| | | | PGDWERARYTYIACINRVWREAVDPYATAVSVNGEFGVVI |
| | | | DWEKTKLAPPSLPLPPLCSPTDAIIYELSIRDFTSHPDSGA |
| | | | VHKGKYLGLAETNTSGPNGTATGLSYVKELGVTHVQLMP |
| | | | FMDFAGVDERDPQAAYNWGYNPLHLYAPEGSYATDPAD |
| | | | PYARIVELKQAIHTLHENGLRVVMDAVYNHVYDREQSPLE |
| | | | KLVPGYYFRYDAYGQPANGTGVGNDIASERRMARRWIVD |
| | | | SVVFWAKEYGIDGFRFDLMGVHDIETMKAVRDALDAIDPS |
| | | | ILVYGEGWDLPTPLPPEQKATMANAKQLPRFAYFNDRFR |
| | | | DAVKGSTFHLPDRGFALGNPGGREQVKLAIAGSLRALGG |
| | | | LFCHPRQSINYVECHDNHTFWDKMEAANHDEPEWLRRK |
| | | | RQKLATAIVLLAQGIPFLHSGQEFYRTKGGDGNSYRSPDA |
| | | | VNQLDWERKSRYEDDVRYVQGLIALRRAHGAFRLATEAE |
| | | | VLRHFTFLEPLPPSVIAYRLHDAAVYGPWEDIIVVHHNEEK |
| | | | ETAIALPDEREWAVVCDGQRCGTTPFGQARGMLRLDGIG |
| | | | TWVLVHPAGHHHHHH |
| EXI89323.1 | Accumulibacter | 302 | MRRRELRNGVLTVALLAASAAFSWAGEELAAVAAENSPK |
| | sp. (strain BA-93) | | VSSAPSAASGQEARYGAHVGADGWVDFVVHAPAATAVD |
| | | | LLLYDDPRARTPQRRVAMTPAGDDWKARVRGSRHGPAL |
| | | | FYMYQASGPREVSPERPFGALFNPAYVLNDPYAYRTENV |
| | | | NYDAFFATVPFTDMTTPVYAGGGKSVVYDHSGDGFPGH |
| | | | VRIAPEDLIVYELHVQDYTATLEGLPAARRGTYLGLARGGL |
| | | | KTPGGLAAGIDHLVELGVNAVELMPVMEYDEQSGNQSGR |
| | | | LNHWGYMTSNFFAPEARYAASPGRQVVELKQLVRALHQ |
| | | | RGIAVFLDVVYNHTAEQSPWVSDGRLAAKCFNLMCLAAD |
| | | | RVYRPTSDGRYYLNNTGTGNDVDFSGGKRYSKQLVRDS |
| | | | LAMWHQIYGIDGFRFDLARILAEGSHDAADWVDNDPRYA |
| | | | TAHLHAEPWDMGGQWWDFMDSDGWSADNNRWAKWL |
| | | | GHYRDKVRRFSQSSLNDPDAFKQLIEGRGSVSRGAGPAA |
| | | | SSRPWRSINLLAVHDGYTLRDCTWFNDSDGSQNCWDSN |
| | | | QDEDLRRQREKLLLGILLTSQGVPVILQGDEFGRSKAGAA |
| | | | AQEGARNSYNYESSTGDLAVNRVNWIDWRLKDGDNSAS |
| | | | PTGPAYGKELFEWTRGLITLRKKWSHFRRHDFARYVHAA |
| | | | TDDRAGPINDGGFSYVWEGPRSGEPTQLAVVWWGKAGE |
| | | | PDLMVIYNEGWEPFKVDNLSHWSRGNWKILARSWHGPG |
| | | | QDMCRGKWKQCPAAGSAVTVAGRSMAILVSDND |
| EXI89323.1 | Accumulibacter | 303 | GEELAAVAAENSPKVSSAPSAASGQEARYGAHVGADGW |
| (6X-His) | sp. (strain BA-93) | | VDFVVHAPAATAVDLLLYDDPRARTPQRRVAMTPAGDDW |
| | | | KARVRGSRHGPALFYMYQASGPREVSPERPFGALFNPAY |
| | | | VLNDPYAYRTENVNYDAFFATVPFTDMTTPVYAGGGKSV |
| | | | VYDHSGDGFPGHVRIAPEDLIVYELHVQDYTATLEGLPAA |
| | | | RRGTYLGLARGGLKTPGGLAAGIDHLVELGVNAVELMPV |
| | | | MEYDEQSGNQSGRLNHWGYMTSNFFAPEARYAASPGR |
| | | | QVVELKQLVRALHQRGIAVFLDVVYNHTAEQSPWVSDGR |
| | | | LAAKCFNLMCLAADRVYRPTSDGRYYLNNTGTGNDVDFS |
| | | | GGKRYSKQLVRDSLAMWHQIYGIDGFRFDLARILAEGSHD |
| | | | AADWVDNDPRYATAHLHAEPWDMGGQWWDFMDSDGW |
| | | | SADNNRWAKWLGHYRDKVRRFSQSSLNDPDAFKQLIEG |
| | | | RGSVSRGAGPAASSRPWRSINLLAVHDGYTLRDCTWFN |
| | | | DSDGSQNCWDSNQDEDLRRQREKLLLGILLTSQGVPVIL |
| | | | QGDEFGRSKAGAAAQEGARNSYNYESSTGDLAVNRVNW |
| | | | IDWRLKDGDNSASPTGPAYGKELFEWTRGLITLRKKWSH |
| | | | FRRHDFARYVHAATDDRAGPINDGGFSYVWEGPRSGEP |
| | | | TQLAVVWWGKAGEPDLMVIYNEGWEPFKVDNLSHWSRG |
| | | | NWKILARSWHGPGQDMCRGKWKQCPAAGSAVTVAGRS |
| | | | MAILVSDNDHHHHHH |
| AAX50293.1 | Chlamydia | 304 | MESLSVRSTIPLPLGAKKLSADRYRFSLFSSQAQQVTLVLL |
| | trachomatis | | DPLSEIHEIPLSSTDHRTGAIWHIEIAGISSEWSYAYKLRGT |
| | serovar A (strain | | DLSSQKFATDSYIADPYSKNIYSPQLFGSPKQEKDYAFSYL |
| | ATCC VR-571B/ | | KHEDFDWEGDTPLHLPKENYFIYEMHVRSFTRDPSSQVS |
| | DSM 19440/ | | HPGTFLGIIEKIDHLKQLGVHAVELLPIFEFDETVHPFKNQD |
| | HAR-13) | | FPHLCNYWGYSSVNFFCPSRRYTYGADPCAPAREFKTLV |
| | | | KALHRAGIEVILDVVFNHTGFEGTSCPLPWIDLESYYMVN |
| | | | DHGDLMNFSGCGNTVNTNTPTTLKWILDALRYWVQEMH |
| | | | VDGFRFDLASVFSRDPQGVPLPLTPILQAISSDSILSETKLI |
| | | | AEPWDAGGLYQLGHFPSISTRWSEWNGCYRDHVKAFLN |
| | | | GDAHQVSSFASRISGSHDIYPNGKPTNSINYICSHDGFTLY |
| | | | DTVAYNDKHNEENGEYNRDGTSANYSYNFGCEGETTDP |
| | | | TICALRERQMKNFFLALFLSQGIPMIQSGDEYGHTAYGNN |
| | | | NHWCLDTKINYFLWDRLAERKELFSFLCQVIALRKAYTELF |
| | | | NTSFLSEDTIVWLNTKGSPREWGADHYLAFELKHLNYSLF |
| | | | VAFYSGNERIEISLPKPRKEHLAYEKIVDSTTGFFSQILSPK |
| | | | LSLEPYSSLVAISRRKTSLESR |
| AAX50293.1 | Chlamydia | 305 | MESLSVRSTIPLPLGAKKLSADRYRFSLFSSQAQQVTLVLL |
| (6X-His) | trachomatis | | DPLSEIHEIPLSSTDHRTGAIWHIEIAGISSEWSYAYKLRGT |
| | serovar A (strain | | DLSSQKFATDSYIADPYSKNIYSPQLFGSPKQEKDYAFSYL |
| | ATCC VR-571B/ | | KHEDFDWEGDTPLHLPKENYFIYEMHVRSFTRDPSSQVS |
| | DSM 19440/ | | HPGTFLGIIEKIDHLKQLGVHAVELLPIFEFDETVHPFKNQD |
| | HAR-13) | | FPHLCNYWGYSSVNFFCPSRRYTYGADPCAPAREFKTLV |
| | | | KALHRAGIEVILDWVFNHTGFEGTSCPLPWIDLESYYMVN |
| | | | DHGDLMNFSGCGNTVNTNTPTTLKWILDALRYWVQEMH |
| | | | VDGFRFDLASVFSRDPQGVPLPLTPILQAISSDSILSETKLI |
| | | | AEPWDAGGLYQLGHFPSISTRWSEWNGCYRDHVKAFLN |
| | | | GDAHQVSSFASRISGSHDIYPNGKPTNSINYICSHDGFTLY |
| | | | DTVAYNDKHNEENGEYNRDGTSANYSYNFGCEGETTDP |
| | | | TICALRERQMKNFFLALFLSQGIPMIQSGDEYGHTAYGNN |
| | | | NHWCLDTKINYFLWDRLAERKELFSFLCQVIALRKAYTELF |
| | | | NTSFLSEDTIVWLNTKGSPREWGADHYLAFELKHLNYSLF |
| | | | VAFYSGNERIEISLPKPRKEHLAYEKIVDSTTGFFSQILSPK |
| | | | LSLEPYSSLVAISRRKTSLESRHHHHHH |
| ABN45630.1 | Streptococcus | 306 | MAFRIFQAYLDDENIITIELEKSFEAYSIHFTLEDKHSSSPLT |
| | sanguinis (strain | | IKNINNQEERIIYTVSANEPIDLTQSYKVYDQDRNHTDLQY |
| | SK36) | | RHIVKKPIFDEIFDYAGDDLGAQYNPQATDFKLWAPISEKV |
| | | | LLHLKDQVHHLKRLDKGVWHIRIEGDLEGSSYSYLHKING |
| | | | KWVEVHDPYALSSDVNSGNSYVINLEKIKKPIKRAKTQLNP |
| | | | TEAVIYEMSVRDFSMQKEIGFSCPGKFASLSESPVVHGQK |
| | | | FGLDYLKELGISHVQLMPVYDFGSVDEKHPELVYNWGYD |
| | | | PVQYNVPDGSFASNPRDPYARILELQAAITAFHNADISVIM |
| | | | DWVYNHVYDANSYAFEKIVPGYFFRLNDMGYRTNGTFCG |
| | | | NDVASEKAMVRRYIKQSVKQWVSLYGFDGFRFDLMGILDI |
| | | | QTMQQIADELKTLYPNIYLYGEGWQMDTGLASERLAHQY |
| | | | NAAQLPDYGFFSDHFRDSLKQTIAQGRQIESKTPASQLEN |
| | | | VLTANVGLTGEAHFTAPQQAINYVECHDNATVFDYFDIVN |
| | | | PAITLRDRLANSRLALHLVLLAQGVPFIHSGQEFFRTKNLID |
| | | | NTYNMPDEINKLDWLRSLHYTEDIAFVKQLIAFRRAHPLLH |
| | | | LKTSAAIKQACQVNWLTDSLLEYKIQDRKESMTIVINFGNQ |
| | | | EAVYENKNKQRLHLQYPAIDAQKPIAPLADSYNLAGKQLIV |
| | | | LKS |
| ABN45630.1 | Streptococcus | 307 | MAFRIFQAYLDDENIITIELEKSFEAYSIHFTLEDKHSSSPLT |
| (6X-His) | sanguinis (strain | | IKNINNQEERIIYTVSANEPIDLTQSYKVYDQDRNHTDLQY |
| | SK36) | | RHIVKKPIFDEIFDYAGDDLGAQYNPQATDFKLWAPISEKV |
| | | | LLHLKDQVHHLKRLDKGVWHIRIEGDLEGSSYSYLHKING |
| | | | KWVEVHDPYALSSDVNSGNSYVINLEKIKKPIKRAKTQLNP |
| | | | TEAVIYEMSVRDFSMQKEIGFSCPGKFASLSESPVVHGQK |
| | | | FGLDYLKELGISHVQLMPVYDFGSVDEKHPELVYNWGYD |
| | | | PVQYNVPDGSFASNPRDPYARILELQAAITAFHNADISVIM |
| | | | DVVYNHVYDANSYAFEKIVPGYFFRLNDMGYRTNGTFCG |
| | | | NDVASEKAMVRRYIKQSVKQWVSLYGFDGFRFDLMGILDI |
| | | | QTMQQIADELKTLYPNIYLYGEGWQMDTGLASERLAHQY |
| | | | NAAQLPDYGFFSDHFRDSLKQTIAQGRQIESKTPASQLEN |
| | | | VLTANVGLTGEAHFTAPQQAINYVECHDNATVFDYFDIVN |
| | | | PAITLRDRLANSRLALHLVLLAQGVPFIHSGQEFFRTKNLID |
| | | | NTYNMPDEINKLDWLRSLHYTEDIAFVKQLIAFRRAHPLLH |
| | | | LKTSAAIKQACQVNWLTDSLLEYKIQDRKESMTIVINFGNQ |
| | | | EAVYENKNKQRLHLQYPAIDAQKPIAPLADSYNLAGKQLIV |
| | | | LKSHHHHHH |
| EFG95061.1 | Fusobacterium | 308 | MYYNFNHYINLGANLEKDGCSFAIYAKNVNSLSLNIFHSSE |
| | nucleatum subsp. | | DTVPYEKHILSPSEHKLGDIWSIFLENIKEGTLYNWEINGM |
| | nucleatum (strain | | AILDPYALAYTGNKTIENKKSIVLARVGTETKHILIPKKNMIIY |
| | ATCC 23726/ | | ESHIGLFTKSPSSNTLNGATYSAFEEKIPYLKNLGINVVEFL |
| | VPI 4351) | | PIFEWDDFTGNLDRESFFLKNVWGYNPINFFALTKKYSSS |
| | | | KDENSANEINEFKKLIFSLHKNGIEVILDVVYNHTAEGGTG |
| | | | GKVYNFKAMGENIFYTKDRENYFTNFSGCGNTLNCNHKV |
| | | | VKDMIIQSLLYWYLEVGVDGFRFDLAPVLGRDSNNQWAR |
| | | | HSLLHELIEHPILSHAKLIAESWDLGGYFVGAMPSGWCEW |
| | | | NGAYRDTVRQFIRGDFGQVPELIKRIFGSVDIFHANKNGY |
| | | | QSSINFICCHDGFTMWDLVSYNLKHNLLNGENNQDGENN |
| | | | NHSYNHGEEGFTENSHIISLRKQQIKNMILILYISQGIPMLL |
| | | | MGDEMGRTQLGNNNAYCQDNPTTWVDWDRKKDFEDVF |
| | | | LFTKNMISLRKSYSIFKKETPLIEGEEVILHGIKLYQPDLSFH |
| | | | SLSIAFQLKDIKSNTDFYIAFNSYTEQLCFELPILENKSWYIL |
| | | | TDTSKVDTCNFQETKCQDTHCCVLPKSSVILISK |
| EFG95061.1 | Fusobacterium | 309 | MYYNFNHYINLGANLEKDGCSFAIYAKNVNSLSLNIFHSSE |
| (6X-His) | nucleatum subsp. | | DTVPYEKHILSPSEHKLGDIWSIFLENIKEGTLYNWEINGM |
| | nucleatum (strain | | AILDPYALAYTGNKTIENKKSIVLARVGTETKHILIPKKNMIIY |
| | ATCC 23726/ | | ESHIGLFTKSPSSNTLNGATYSAFEEKIPYLKNLGINVVEFL |
| | VPI 4351) | | PIFEWDDFTGNLDRESFFLKNVWGYNPINFFALTKKYSSS |
| | | | KDENSANEINEFKKLIFSLHKNGIEVILDVVYNHTAEGGTG |
| | | | GKVYNFKAMGENIFYTKDRENYFTNFSGCGNTLNCNHKV |
| | | | VKDMIIQSLLYWYLEVGVDGFRFDLAPVLGRDSNNQWAR |
| | | | HSLLHELIEHPILSHAKLIAESWDLGGYFVGAMPSGWCEW |
| | | | NGAYRDTVRQFIRGDFGQVPELIKRIFGSVDIFHANKNGY |
| | | | QSSINFICCHDGFTMWDLVSYNLKHNLLNGENNQDGENN |
| | | | NHSYNHGEEGFTENSHIISLRKQQIKNMILILYISQGIPMLL |
| | | | MGDEMGRTQLGNNNAYCQDNPTTWVDWDRKKDFEDVF |
| | | | LFTKNMISLRKSYSIFKKETPLIEGEEVILHGIKLYQPDLSFH |
| | | | SLSIAFQLKDIKSNTDFYIAFNSYTEQLCFELPILENKSWYIL |
| | | | TDTSKVDTCNFQETKCQDTHCCVLPKSSVILISKHHHHHH |
| ETT26794.1 | Rhodococcus | 310 | MEPTEPSTSTEALPADIPVWPGSAYPLGATYDGAGTNFAL |
| | rhodochrous | | FSEVAEKVELCLIAKDGTETRVPIEEVDGNVWHVYLPTVT |
| | ATCC 21198 | | PGQRYGYRVHGPYDPAQGLRCDPSKLLLDPYGKAYDGA |
| | | | FDGDASLFSYAMVGAEDAPSEPEHSAGAEEAQSAPEPAA |
| | | | RAAEDAGAEAAGDTAGAEAAGNTTADEADDSDGTGSGE |
| | | | TGDDGTGDDGTGDDTAAESAPEPAAPANPDFPQLDSLG |
| | | | HTMTTVVINPFFDWASDRHPRRPYYETVIYEAHVKGMTAT |
| | | | HPGVPEQLRGTYAGLAHPVIIDHLQSLGVTAIELMPVHQF |
| | | | MHDQVLLDRGLRNYWGYNTFGFLAPHLDYSSASNPSGV |
| | | | VAEFKAMVRAYHEAGIEVILDVVYNHTAEGNHLGPTISFR |
| | | | GIDNAAYYRLVDDDKSYYMDYTGTGNSLNARHPHTLQLIM |
| | | | DSLRYWVTEMHVDGFRFDLASTLARELHDVDRLSAFFDL |
| | | | VQQDPIVSQVKLIAEPWDVGEGGYQVGNFPGLWTEWNG |
| | | | KYRDTVRDYWRGEPATLGEFASRLTGSSDLYEATGRRPS |
| | | | ASINFVTAHDGFTLADLVSYNEKHNDANGENNMDGESHN |
| | | | RSWNCGAEGPTDDPEVLALRQRQSRNILATLMLSQGTPM |
| | | | IAHGDEFGRTQQGNNNVYCQDNELSWMDWSLAETNADL |
| | | | LDFTRNVIALRRSHPVFRRRRFFEGRPIRSGEQSRDIAWR |
| | | | TPAGEEMTPEDWDSGFGKSLSVFLNGEGIHEPNQRGER |
| | | | WVDDSFLLCFNAHDEPIDFVTPDGPHAQEWSVALDTDVP |
| | | | DGLREQVVTAGGSVRVQARSLLVLRRTA |
| ETT26794.1 | Rhodococcus | 311 | MEPTEPSTSTEALPADIPVWPGSAYPLGATYDGAGTNFAL |
| (6X-His) | rhodochrous | | FSEVAEKVELCLIAKDGTETRVPIEEVDGNVWHVYLPTVT |
| | ATCC 21198 | | PGQRYGYRVHGPYDPAQGLRCDPSKLLLDPYGKAYDGA |
| | | | FDGDASLFSYAMVGAEDAPSEPEHSAGAEEAQSAPEPAA |
| | | | RAAEDAGAEAAGDTAGAEAAGNTTADEADDSDGTGSGE |
| | | | TGDDGTGDDGTGDDTAAESAPEPAAPANPDFPQLDSLG |
| | | | HTMTTVVINPFFDWASDRHPRRPYYETVIYEAHVKGMTAT |
| | | | HPGVPEQLRGTYAGLAHPVIIDHLQSLGVTAIELMPVHQF |
| | | | MHDQVLLDRGLRNYWGYNTFGFLAPHLDYSSASNPSGV |
| | | | VAEFKAMVRAYHEAGIEVILDVVYNHTAEGNHLGPTISFR |
| | | | GIDNAAYYRLVDDDKSYYMDYTGTGNSLNARHPHTLQLIM |
| | | | DSLRYWVTEMHVDGFRFDLASTLARELHDVDRLSAFFDL |
| | | | VQQDPIVSQVKLIAEPWDVGEGGYQVGNFPGLWTEWNG |
| | | | KYRDTVRDYWRGEPATLGEFASRLTGSSDLYEATGRRPS |
| | | | ASINFVTAHDGFTLADLVSYNEKHNDANGENNMDGESHN |
| | | | RSWNCGAEGPTDDPEVLALRQRQSRNILATLMLSQGTPM |
| | | | IAHGDEFGRTQQGNNNVYCQDNELSWMDWSLAETNADL |
| | | | LDFTRNVIALRRSHPVFRRRRFFEGRPIRSGEQSRDIAWR |
| | | | TPAGEEMTPEDWDSGFGKSLSVFLNGEGIHEPNQRGER |
| | | | WVDDSFLLCFNAHDEPIDFVTPDGPHAQEWSVALDTDVP |
| | | | DGLREQVVTAGGSVRVQARSLLVLRRTAHHHHHH |
| CBA29739.1 | Curvibacter | 312 | MLPGNAAEWGATLTPEGVNFTLAAPNATSVDLCLFDEAG |
| | symbiont subsp. | | VTELQRLPLPAKTGDVWHGLLPAGRAGQVYGFRVHGPW |
| | Hydra | | APERGHRFNPAKLLLDPYAREVLGRYDGSDLHWAHAPDS |
| | magnipapillata | | ARGRQLPDSRDNAATALKARVCAPLPAANPGPVVLPKQR |
| | | | VMYELHVKGFTRLHPGVPETLRGSYAGLAHPAAIAHLKAM |
| | | | GVTTLSLMPVAFRADEERLQRLGLSNYWGYSPIAWSAPE |
| | | | SRYWSGTAGSTPRTEFRAMVDAVHAAGIEVILDVVYNHT |
| | | | GETDEFGPLLSLRGIDNGTYYHLEPGDASRYLNWTGCGN |
| | | | CVNLNHPVVLRTVMDSLRAWVTDYGVDGFRFDLAPVLAR |
| | | | GTAETDYRFNPHAPLLMAIAQDPTLRNCVMVAEPWDIGP |
| | | | GGYQLGAFPAGWLEWNDRFRDTQRSAWLQHSTHRGAL |
| | | | ANRLAGSADAFSPLRRAAHSGVNFVTAHDGFNLMDVVSY |
| | | | CHRHNEANGEHNRDGHGHNLSVNHGVEGGSEDEQVVQ |
| | | | ARARHRRTLLAVTLFSLGTPMLLAGDELGHSQCGNNNAY |
| | | | CQDNATTWLNWEAARHDLSSFVARAIALRRALPALQATG |
| | | | WWRSHTGEAGSGPVAMWALPDGRTVEAGDWEAPQGG |
| | | | PLAVHLQLGSSGAVLLLLNPSASAQAFTLPDGEWHLCLDT |
| | | | GSESVGTEPASTVSPLFTVQADSLVLLSAVQPVF |
| CBA29739.1 | Curvibacter | 313 | MLPGNAAEWGATLTPEGVNFTLAAPNATSVDLCLFDEAG |
| (6X-His) | symbiont subsp. | | VTELQRLPLPAKTGDVWHGLLPAGRAGQVYGFRVHGPW |
| | Hydra | | APERGHRFNPAKLLLDPYAREVLGRYDGSDLHWAHAPDS |
| | magnipapillata | | ARGRQLPDSRDNAATALKARVCAPLPAANPGPVVLPKQR |
| | | | VMYELHVKGFTRLHPGVPETLRGSYAGLAHPAAIAHLKAM |
| | | | GVTTLSLMPVAFRADEERLQRLGLSNYWGYSPIAWSAPE |
| | | | SRYWSGTAGSTPRTEFRAMVDAVHAAGIEVILDVVYNHT |
| | | | GETDEFGPLLSLRGIDNGTYYHLEPGDASRYLNWTGCGN |
| | | | CVNLNHPVVLRTVMDSLRAWVTDYGVDGFRFDLAPVLAR |
| | | | GTAETDYRFNPHAPLLMAIAQDPTLRNCVMVAEPWDIGP |
| | | | GGYQLGAFPAGWLEWNDRFRDTQRSAWLQHSTHRGAL |
| | | | ANRLAGSADAFSPLRRAAHSGVNFVTAHDGFNLMDVVSY |
| | | | CHRHNEANGEHNRDGHGHNLSVNHGVEGGSEDEQVVQ |
| | | | ARARHRRTLLAVTLFSLGTPMLLAGDELGHSQCGNNNAY |
| | | | CQDNATTWLNWEAARHDLSSFVARAIALRRALPALQATG |
| | | | WWRSHTGEAGSGPVAMWALPDGRTVEAGDWEAPQGG |
| | | | PLAVHLQLGSSGAVLLLLNPSASAQAFTLPDGEWHLCLDT |
| | | | GSESVGTEPASTVSPLFTVQADSLVLLSAVQPVFHHHHH |
| | | | H |
| CBL56593.1 | Propionibacterium | 314 | MGRSRGTTPRCREDRDAPEAHATDRVARRKYTWMTEIN |
| | freudenreichii | | RSDAAHVLGAHLSADGCHFGLWAPRAERVELALVDGDG |
| | subsp. shermanii | | STQRNVDMTLGGGAWSVFVPDVVAGQRYGFRVHAQWD |
| | (strain ATCC | | PDQGLRANPAKLLVDPYARAITAGVDYSGPIFDHVPGSYF |
| | 9614/DSM 4902/ | | EPDTRDSAGSVPLSVVVADSPAPEPIAERRPLEECVIYET |
| | CIP 103027/ | | HVKGLTQLHPTVPEHLRGRFAGVAYPAVTEHLKSLGVNA |
| | NCIMB 8099/ | | VEFLPVHHFISEPFVMGRGLSNYWGYNSLGFFAPHAAYC |
| | CIRM-BIA1) | | SVGTEGDQVAEFKEMVTALHRAGIEVILDVVYNHTCEGNH |
| | | | EGPTLSFRGIDHRGYYRLTDDLRNDYDITGTGNSVNTAHA |
| | | | DVLAMVVDSMRYWVQEMGVDGFRFDLATELIRDGEHHV |
| | | | DQNHDFKKLIAQDPAFKGVKMIAEPWDLGPYGYQVGNW |
| | | | GPGWSEWNDRFRGYMRDYWRGQVDGVDELATRLSGSA |
| | | | DLFDHDDRPPSSSINFFDAHDGFPLRDLVTYNEKHNEAN |
| | | | GEDNRDGSDDNRSWNCGVEGETADEVINALRHRQIRNM |
| | | | VATLMLSDGVPMYCAGDEMGRTQQGNNNAYCQDGPIN |
| | | | WLRWDQMEEWGDVLDTVRTFTDLRMSTPLLHANDYRYR |
| | | | TEVTDPTGAGLGRYELAWMNGSSGEMGEADWHDGSRR |
| | | | LLGMYVSDASSVAYLSWFYSGDQPIQVQMPPAPWGESF |
| | | | HIVASTCEDGEVPDADLAPGDSFTMPPRTVVTMRVAVMT |
| | | | TAPVPDDQPVTDQEIATGDPHIPDTPQAAAPTS |
| CBL56593.1 | Propionibacteriu | 315 | MGRSRGTTPRCREDRDAPEAHATDRVARRKYTWMTEIN |
| (6X- | m freudenreichii | | RSDAAHVLGAHLSADGCHFGLWAPRAERVELALVDGDG |
| His) | subsp. shermanii | | STQRNVDMTLGGGAWSVFVPDVVAGQRYGFRVHAQWD |
| | (strain ATCC | | PDQGLRANPAKLLVDPYARAITAGVDYSGPIFDHVPGSYF |
| | 9614/DSM 4902 | | EPDTRDSAGSVPLSVVVADSPAPEPIAERRPLEECVIYET |
| | / CIP 103027/ | | HVKGLTQLHPTVPEHLRGRFAGVAYPAVTEHLKSLGVNA |
| | NCIMB 8099/ | | VEFLPVHHFISEPFVMGRGLSNYWGYNSLGFFAPHAAYC |
| | CIRM-BIA1) | | SVGTEGDQVAEFKEMVTALHRAGIEVILDVVYNHTCEGNH |
| | | | EGPTLSFRGIDHRGYYRLTDDLRNDYDITGTGNSVNTAHA |
| | | | DVLAMVVDSMRYWVQEMGVDGFRFDLATELIRDGEHHV |
| | | | DQNHDFKKLIAQDPAFKGVKMIAEPWDLGPYGYQVGNW |
| | | | GPGWSEWNDRFRGYMRDYWRGQVDGVDELATRLSGSA |
| | | | DLFDHDDRPPSSSINFFDAHDGFPLRDLVTYNEKHNEAN |
| | | | GEDNRDGSDDNRSWNCGVEGETADEVINALRHRQIRNM |
| | | | VATLMLSDGVPMYCAGDEMGRTQQGNNNAYCQDGPIN |
| | | | WLRWDQMEEWGDVLDTVRTFTDLRMSTPLLHANDYRYR |
| | | | TEVTDPTGAGLGRYELAWMNGSSGEMGEADWHDGSRR |
| | | | LLGMYVSDASSVAYLSWFYSGDQPIQVQMPPAPWGESF |
| | | | HIVASTCEDGEVPDADLAPGDSFTMPPRTVVTMRVAVMT |
| | | | TAPVPDDQPVTDQEIATGDPHIPDTPQAAAPTSHHHHHH |
| AEH13197.1 | Shewanella | 316 | MQKPTADDIKLNPDAKHNPSQNEKWALGAGEPFPLGASV |
| | baltica OS117 | | EGDGVNFALFSANATAVELCLFDDNGEQEIARIALTEQTQ |
| | | | QIWHVFVHGLKAGQLYGYRVYGSYEPLLGHRFNPNKLLL |
| | | | DPYARQLVGEYSDHESNYGYELNHKDEDLSFSKLDNAAH |
| | | | VPKCKVVDIRPLIDIAAKQSIQPVATRFQPNPLERSIIYEMH |
| | | | VKGFTAAHPEIEPNKRATFAGLATQPAIDYLAELGVTSVEL |
| | | | LPVQAFFTEPFLLEKKLTNYWGYNSIGFFAPEPSYLSSDDI |
| | | | GEFRAMVDALHGAGIEVILDVVYNHSAEGSRLGPTFSFRG |
| | | | IDNLSYYRLHPNDKRFYINDTGCGNTLNINHPRMLQLVLDS |
| | | | LRYWVEIMGVDGFRFDLAACLGREAYGFDPGSGFFDALL |
| | | | QDPVLCRVKLIAEPWDIGPGGYQLGNFPVAFSEWNDRYR |
| | | | DTMRRFWRGDHSMLPEFARRFHGSGDFFEHGGRPPATS |
| | | | LNFLTSHDGFTLKDLVSYTQRHNLANGEDNRDGHQENFS |
| | | | HHYGIEGETEDSAILALRSRQQRNLLTTLFLSQGVPMLLS |
| | | | GDETGRTQQGNNNAYCQDNELNWFDWSAKGMDTELLA |
| | | | FTQQLIALRKRFPLLCAKRFIHEQLLAESLADTGARLDWFS |
| | | | RQGEQMTKSLWTESMCRSLSVVLSGDLEGQGKQQALLL |
| | | | MVNADDNPLAFTPPTFEHLSPWQCLIHTQTEPLDAQISTT |
| | | | RYLLQDRSLMLFHADLIFKK |
| AEH13197.1 | Shewanella | 317 | MQKPTADDIKLNPDAKHNPSQNEKWALGAGEPFPLGASV |
| (6X-His) | baltica OS117 | | EGDGVNFALFSANATAVELCLFDDNGEQEIARIALTEQTQ |
| | | | QIWHVFVHGLKAGQLYGYRVYGSYEPLLGHRFNPNKLLL |
| | | | DPYARQLVGEYSDHESNYGYELNHKDEDLSFSKLDNAAH |
| | | | VPKCKVVDIRPLIDIAAKQSIQPVATRFQPNPLERSIIYEMH |
| | | | VKGFTAAHPEIEPNKRATFAGLATQPAIDYLAELGVTSVEL |
| | | | LPVQAFFTEPFLLEKKLTNYWGYNSIGFFAPEPSYLSSDDI |
| | | | GEFRAMVDALHGAGIEVILDVVYNHSAEGSRLGPTFSFRG |
| | | | IDNLSYYRLHPNDKRFYINDTGCGNTLNINHPRMLQLVLDS |
| | | | LRYWVEIMGVDGFRFDLAACLGREAYGFDPGSGFFDALL |
| | | | QDPVLCRVKLIAEPWDIGPGGYQLGNFPVAFSEWNDRYR |
| | | | DTMRRFWRGDHSMLPEFARRFHGSGDFFEHGGRPPATS |
| | | | LNFLTSHDGFTLKDLVSYTQRHNLANGEDNRDGHQENFS |
| | | | HHYGIEGETEDSAILALRSRQQRNLLTTLFLSQGVPMLLS |
| | | | GDETGRTQQGNNNAYCQDNELNWFDWSAKGMDTELLA |
| | | | FTQQLIALRKRFPLLCAKRFIHEQLLAESLADTGARLDWFS |
| | | | RQGEQMTKSLWTESMCRSLSVVLSGDLEGQGKQQALLL |
| | | | MVNADDNPLAFTPPTFEHLSPWQCLIHTQTEPLDAQISTT |
| | | | RYLLQDRSLMLFHADLIFKKHHHHHH |
| CAB9494372. | Alteromonas | 318 | MKEISEIVQIVESGVGNAHPLGATLTHDGSNFAVYAPDAK |
| 1 | macleodii | | AVVLCFFNCDTEEAISEHPLPEKTGDVWHGHFSGVSAGQ |
| | | | YYGYRVERGEVGLHAVPTDKLLIDPYAKKISRAIKWDARQ |
| | | | YKHDSQFMIPKCIVIDHNDYATNHARPPVIPKHKRVVYEAH |
| | | | VKGLTKLHPEVPKAHRGKFIGAAHPSVIKHIKALGVTTVQF |
| | | | MPLCSFMPEPFITDKGLTNYWGYNPVNFFAPEPRYGVSD |
| | | | ALAELKSMIDAYHSAGLEVIVDVVFNHTAEAGGGGPILSYK |
| | | | GFCPNQAYLLEQTKNGELVYSNHSGCGNTVNTAQPFMM |
| | | | GLILDAMRHWVSVIGVDGFRFDLAVCLGREPQQYNKKSG |
| | | | LLRAISSDPVLKDKVLLAEPWDIGPNGYQVGNFPSPWLEV |
| | | | NDKYRDTVRAFWRGDGGVTADFATRLMGSRDIFHKGRR |
| | | | HISTSVNNVTYHDGFTLHDMVTYAERHNLDNLEENRDGH |
| | | | GHNLSANYGVEGETNDERILAMRERQKRNLFATLIFSQGT |
| | | | PHILGGDELSRTQNGNNNAYCQDNPISWMNWELNKRKQ |
| | | | DFLSFCQYVVRLRQSSLLLSELKLHDDTFTLSRNVKEINW |
| | | | YKPDGSDKASEDWNAHHNKAFGVEIKGCVMSQREPDQK |
| | | | PEHWFLCVNASDSDVRFHLPTVLPKGGWTMHLDTRYSSL |
| | | | EEQPSICIQKVFLQASKSLTLFSFSQFSE |
| CAB9494372. | Alteromonas | 319 | MKEISEIVQIVESGVGNAHPLGATLTHDGSNFAVYAPDAK |
| 1 | macleodii | | AVVLCFFNCDTEEAISEHPLPEKTGDVWHGHFSGVSAGQ |
| (6X-His) | | | YYGYRVERGEVGLHAVPTDKLLIDPYAKKISRAIKWDARQ |
| | | | YKHDSQFMIPKCIVIDHNDYATNHARPPVIPKHKRVVYEAH |
| | | | VKGLTKLHPEVPKAHRGKFIGAAHPSVIKHIKALGVTTVQF |
| | | | MPLCSFMPEPFITDKGLTNYWGYNPVNFFAPEPRYGVSD |
| | | | ALAELKSMIDAYHSAGLEVIVDVVFNHTAEAGGGGPILSYK |
| | | | GFCPNQAYLLEQTKNGELVYSNHSGCGNTVNTAQPFMM |
| | | | GLILDAMRHWVSVIGVDGFRFDLAVCLGREPQQYNKKSG |
| | | | LLRAISSDPVLKDKVLLAEPWDIGPNGYQVGNFPSPWLEV |
| | | | NDKYRDTVRAFWRGDGGVTADFATRLMGSRDIFHKGRR |
| | | | HISTSVNNVTYHDGFTLHDMVTYAERHNLDNLEENRDGH |
| | | | GHNLSANYGVEGETNDERILAMRERQKRNLFATLIFSQGT |
| | | | PHILGGDELSRTQNGNNNAYCQDNPISWMNWELNKRKQ |
| | | | DFLSFCQYVVRLRQSSLLLSELKLHDDTFTLSRNVKEINW |
| | | | YKPDGSDKASEDWNAHHNKAFGVEIKGCVMSQREPDQK |
| | | | PEHWFLCVNASDSDVRFHLPTVLPKGGWTMHLDTRYSSL |
| | | | EEQPSICIQKVFLQASKSLTLFSFSQFSEHHHHHH |
| AAF09848 | Deinococcus | 320 | MPYKSLTLRPGSPFPLGATWDGKGTNFALYSENATGVEL |
| | radiodurans R1 | | CLFDAEGHETRFPLTEQTAFVWHGYLPGIQPGQRYGYRV |
| | | | HGEYAPEKGLRFNPNVVLLDPYAKALDGTEQFDRGVFGY |
| | | | VAGGEDDSQMQEEEQRGAPLGLVVDPMFNWVGDQKPGI |
| | | | PFHQSVIYEAHVKGLTMTHPDVPEELRGTYAGVATPAILD |
| | | | YLRDLGITAIEFLPVHQHVDDPFLLDKGLTNYWGYSTLNFF |
| | | | APDVRYSAEARKGNPSGAVPEFKNMVRALHDAGIEVILDV |
| | | | VYNHTAEGNHMGPTMSFKGIDNPTYYRLVADDQRFYFDY |
| | | | TGTGNSLNVRHPQTLQLIMDSLRYWVTEMHVDGFRFDLA |
| | | | STLARGLHEVDQLSGFFTIIHQDPIISQVKLIAEPWDVGEG |
| | | | GYQVGNFPVNWAEWNGIYRDDMRSFWKGEGGLASEIGY |
| | | | RITGSSDLYEFNGRKPYASINFVTAHDGFTLRDSVTYEQK |
| | | | HNEANGEGNNDGHNHNITWNCGVEGPTDDPEINRLRGQ |
| | | | QMRNFLATLLLGQGTPMILGGDEFGRTQGGNNNAYCQD |
| | | | NDISWYDWEKVDEELLAFTRKLIALRKAHPSLHRRKFFAG |
| | | | RNIRGEDVRDIVWLRFDGAEMSDEDWNNPQTQSLGMFL |
| | | | AGDGLADVDAEGKPLTDDHLLLLLSSSYVDLPFKMPDLGG |
| | | | CGEWDLLLDTSDDGAEEKVAAGGETTLRGRSVKLYRCQV |
| | | | PEKELEKLAEPAEA |
| AAF09848 | Deinococcus | 321 | MPYKSLTLRPGSPFPLGATWDGKGTNFALYSENATGVEL |
| (6X-His) | radiodurans R1 | | CLFDAEGHETRFPLTEQTAFVWHGYLPGIQPGQRYGYRV |
| | | | HGEYAPEKGLRFNPNVVLLDPYAKALDGTEQFDRGVFGY |
| | | | VAGGEDDSQMQEEEQRGAPLGLVVDPMFNWVGDQKPGI |
| | | | PFHQSVIYEAHVKGLTMTHPDVPEELRGTYAGVATPAILD |
| | | | YLRDLGITAIEFLPVHQHVDDPFLLDKGLTNYWGYSTLNFF |
| | | | APDVRYSAEARKGNPSGAVPEFKNMVRALHDAGIEVILDV |
| | | | VYNHTAEGNHMGPTMSFKGIDNPTYYRLVADDQRFYFDY |
| | | | TGTGNSLNVRHPQTLQLIMDSLRYWVTEMHVDGFRFDLA |
| | | | STLARGLHEVDQLSGFFTIIHQDPIISQVKLIAEPWDVGEG |
| | | | GYQVGNFPVNWAEWNGIYRDDMRSFWKGEGGLASEIGY |
| | | | RITGSSDLYEFNGRKPYASINFVTAHDGFTLRDSVTYEQK |
| | | | HNEANGEGNNDGHNHNITWNCGVEGPTDDPEINRLRGQ |
| | | | QMRNFLATLLLGQGTPMILGGDEFGRTQGGNNNAYCQD |
| | | | NDISWYDWEKVDEELLAFTRKLIALRKAHPSLHRRKFFAG |
| | | | RNIRGEDVRDIVWLRFDGAEMSDEDWNNPQTQSLGMFL |
| | | | AGDGLADVDAEGKPLTDDHLLLLLSSSYVDLPFKMPDLGG |
| | | | CGEWDLLLDTSDDGAEEKVAAGGETTLRGRSVKLYRCQV |
| | | | PEKELEKLAEPAEAHHHHHH |
| CAA31754.1 | Pseudomonas | 322 | MKCPKILAALLGCAVLAGVPAMPAHAAINSMSLGASYDAQ |
| | amyloderamosa | | QANITFRVYSSQATRIVLYLYSAGYGVQESATYTLSPAGS |
| | | | GVWAVTVPVSSIKAAGITGAVYYGYRAWGPNWPYASNW |
| | | | GKGSQAGFVSDVDANGDRFNPNKLLLDPYAQEVSQDPL |
| | | | NPSNQNGNVFASGASYRTTDSGIYAPKGVVLVPSTQSTG |
| | | | TKPTRAQKDDVIYEVHVRGFTEQDTSIPAQYRGTYYGAGL |
| | | | KASYLASLGVTAVEFLPVQETQNDANDVVPNSDANQNYW |
| | | | GYMTENYFSPDRRYAYNKAAGGPTAEFQAMVQAFHNAGI |
| | | | KVYMDVVYNHTAEGGTWTSSDPTTATIYSWRGLDNATYY |
| | | | ELTSGNQYFYDNTGIGANFNTYNTVAQNLIVDSLAYWANT |
| | | | MGVDGFRFDLASVLGNSCLNGAYTASAPNCPNGGYNFD |
| | | | AADSNVAINRILREFTVRPAAGGSGLDLFAEPWAIGGNSY |
| | | | QLGGFPQGWSEWNGLFRDSLRQAQNELGSMTIYVTQDA |
| | | | NDFSGSSNLFQSSGRSPWNSINFIDVHDGMTLKDVYSCN |
| | | | GANNSQAWPYGPSDGGTSTNYSWDQGMSAGTGAAVDQ |
| | | | RRAARTGMAFEMLSAGTPLMQGGDEYLRTLQCNNNAYN |
| | | | LDSSANWLTYSWTTDQSNFYTFAQRLIAFRKAHPALRPSS |
| | | | WYSGSQLTWYQPSGAVADSNYWNNTSNYAIAYAINGPSL |
| | | | GDSNSIYVAYNGWSSSVTFTLPAPPSGTQWYRVTDTCD |
| | | | WNDGASTFVAPGSETLIGGAGTTYGQCGQSLLLLISK |
| CAA31754.1 | Pseudomonas | 323 | AINSMSLGASYDAQQANITFRVYSSQATRIVLYLYSAGYG |
| (6X-His) | amyloderamosa | | VQESATYTLSPAGSGVWAVTVPVSSIKAAGITGAVYYGYR |
| | | | AWGPNWPYASNWGKGSQAGFVSDVDANGDRFNPNKLL |
| | | | LDPYAQEVSQDPLNPSNQNGNVFASGASYRTTDSGIYAP |
| | | | KGVVLVPSTQSTGTKPTRAQKDDVIYEVHVRGFTEQDTSI |
| | | | PAQYRGTYYGAGLKASYLASLGVTAVEFLPVQETQNDAN |
| | | | DVVPNSDANQNYWGYMTENYFSPDRRYAYNKAAGGPTA |
| | | | EFQAMVQAFHNAGIKVYMDVVYNHTAEGGTWTSSDPTTA |
| | | | TIYSWRGLDNATYYELTSGNQYFYDNTGIGANFNTYNTVA |
| | | | QNLIVDSLAYWANTMGVDGFRFDLASVLGNSCLNGAYTA |
| | | | SAPNCPNGGYNFDAADSNVAINRILREFTVRPAAGGSGLD |
| | | | LFAEPWAIGGNSYQLGGFPQGWSEWNGLFRDSLRQAQN |
| | | | ELGSMTIYVTQDANDFSGSSNLFQSSGRSPWNSINFIDVH |
| | | | DGMTLKDVYSCNGANNSQAWPYGPSDGGTSTNYSWDQ |
| | | | GMSAGTGAAVDQRRAARTGMAFEMLSAGTPLMQGGDE |
| | | | YLRTLQCNNNAYNLDSSANWLTYSWTTDQSNFYTFAQRL |
| | | | IAFRKAHPALRPSSWYSGSQLTWYQPSGAVADSNYWNN |
| | | | TSNYAIAYAINGPSLGDSNSIYVAYNGWSSSVTFTLPAPPS |
| | | | GTQWYRVTDTCDWNDGASTFVAPGSETLIGGAGTTYGQ |
| | | | CGQSLLLLISKHHHHHH |
| AAF09848 | Frankia sp. | 324 | MIRPGSATPLGVWWDGEGVNVAVVAPGADAVDFCVFDG |
| | Ea1.12 | | QNGSGGQNGSGATGRTDLGTDPVRGTDGGAETTSGGE |
| | | | TRYRLPERDGGVWHGYIPGVGPGQAYGLRAHGPYAPGR |
| | | | GERYNPAKLLLDPYARRVTGSFVPHPAVHGYVAGDPYGQ |
| | | | DPDDRDSAPYVPKGVVTGPVTRPPAGSVTGSAAGSVTG |
| | | | PDPAANRPRTAWADTILYELHVRGFTMLHPGVPERLRGT |
| | | | YAGLAHPAVVDHLLRIGVTAVELLPVHAHISETTLLENGRS |
| | | | NYWGYNTLAFFAPHPGYAATDDPVAEFRAMVAALHDAGI |
| | | | EVLLDVVYNHTAEGSERGPTLSLRGLDNIAYYRVEPTDPR |
| | | | RYRDVTGCGNTVDATSPHVVRLICDSLRYWVSEMGVDGF |
| | | | RFDLATALARSPDGFEPAAPLLTAIQADPLLSSVKLIAEPW |
| | | | DLGWGGYQVGAFPAPWAEWNGRFRDTLRDIFSDRTGSV |
| | | | ADLGYRITGSSDIYEHSGRRPWASVNFVTAHDGFPLADLV |
| | | | SYNEKHNEANGEGNRDGESENRSSNHGAEGPTDDPKILT |
| | | | NRRRARRALISTLLLSAGVPMLLAGDELGRSQGGNNNAY |
| | | | CQDNAVSWLAWPNGTAGTPEAGGPGEVPAGEVPAADPA |
| | | | GPDPALVTLVGGLSRLRRAAPVLRRQRFFRGGAPTPQRL |
| | | | PDITWFRQDGAVMSAADWNAPQVAILVAHLAGEGIEWTD |
| | | | AAGTPITGESLLLVIHPEGEDRTVVLPGAPWATWYDLLLD |
| | | | TAADDLAGFPDTITEPRRTLAAGAQLDVAGRTVLILRALGG |
| | | | SDCSVAAE |
| AAF09848 | Frankia sp. | 325 | MIRPGSATPLGVWWDGEGVNVAVVAPGADAVDFCVFDG |
| (6X-His) | Ea1.12 | | QNGSGGQNGSGATGRTDLGTDPVRGTDGGAETTSGGE |
| | | | TRYRLPERDGGVWHGYIPGVGPGQAYGLRAHGPYAPGR |
| | | | GERYNPAKLLLDPYARRVTGSFVPHPAVHGYVAGDPYGQ |
| | | | DPDDRDSAPYVPKGVVTGPVTRPPAGSVTGSAAGSVTG |
| | | | PDPAANRPRTAWADTILYELHVRGFTMLHPGVPERLRGT |
| | | | YAGLAHPAVVDHLLRIGVTAVELLPVHAHISETTLLENGRS |
| | | | NYWGYNTLAFFAPHPGYAATDDPVAEFRAMVAALHDAGI |
| | | | EVLLDVVYNHTAEGSERGPTLSLRGLDNIAYYRVEPTDPR |
| | | | RYRDVTGCGNTVDATSPHWVRLICDSLRYWVSEMGVDGF |
| | | | RFDLATALARSPDGFEPAAPLLTAIQADPLLSSVKLIAEPW |
| | | | DLGWGGYQVGAFPAPWAEWNGRFRDTLRDIFSDRTGSV |
| | | | ADLGYRITGSSDIYEHSGRRPWASVNFVTAHDGFPLADLV |
| | | | SYNEKHNEANGEGNRDGESENRSSNHGAEGPTDDPKILT |
| | | | NRRRARRALISTLLLSAGVPMLLAGDELGRSQGGNNNAY |
| | | | CQDNAVSWLAWPNGTAGTPEAGGPGEVPAGEVPAADPA |
| | | | GPDPALVTLVGGLSRLRRAAPVLRRQRFFRGGAPTPQRL |
| | | | PDITWFRQDGAVMSAADWNAPQVAILVAHLAGEGIEWTD |
| | | | AAGTPITGESLLLVIHPEGEDRTVVLPGAPWATWYDLLLD |
| | | | TAADDLAGFPDTITEPRRTLAAGAQLDVAGRTVLILRALGG |
| | | | SDCSVAAEHHHHHH |
| ARBA000 | Linderina | 326 | MSVSRFSTPVEPEHPPFMVWNLDLNEDGTTPHDKSFIRIP |
| 20723 | pennispora | | SVLNQSCAVRFRILIGSTASIDAVLHTNYPLDGSDYVRTKF |
| | | | HSKRFNFNNSTELICEFRIQRPGPYQYYVTYKSVDDEESD |
| | | | YTDCISCDSDEVPTLERVYRVFKDRRTPTSYFLVDPQLTL |
| | | | GGQHLALDGIALQSVSPKWLGPMKGWSKHLANSSKMGY |
| | | | NMLHFIPMQQRGGSDSPYSLYNQLELSDDLFEGEKLSKE |
| | | | EKDQRLRTVCLEMHHKHHLLGITDMVWNHTAYNSEWLC |
| | | | DHPEAGFNLVNSPHLRSAYELDAKLCEFSKNLPEYGFDR |
| | | | MVSTVAQVDSLMEGVNTHVIQPLKLWEFYVVDVEGTVQA |
| | | | VSDAWDKAGVEMSADDIAKATELQGDERNEWLQTFVINN |
| | | | DPHTLSTRHGRTIDSERAVAVLRLLNGNDKKQGLEQLRRL |
| | | | LDLINLPYYREYDSDVARIRKNVGERVKYERLDRGSWKFG |
| | | | KPVDDDFKIVDPLFTTVTPRGGDSGAGKVNEDLYHLANN |
| | | | GWIWGGNPLDNFAGPQSKAYLQREVIVWGDCVKLNYGT |
| | | | QASDNPWLWEHMRQYTLSMAKAFHGFRIDNCHSTPIELA |
| | | | EYLLDQARKVKPDLYVIAELFTGSEATDQIFVQRLGINSLIR |
| | | | EAMQAWDSHEMSRLAHRHGGRPVGSLAVDCLGEPGFFT |
| | | | DDEHGGTRVDGIVAPLSASLPHAIFFDCTHDNEVPAQKRT |
| | | | MQDALPNAAIVAMTACATGSNRGYDELYPELLNIVHESRQ |
| | | | YAPLEDPLSVGLGEAKAKLNQLHAEMAQFQEVHVHHENE |
| | | | FVTVQRMHPVTRHGVLMVAHCAFRGASENSHFENPRLY |
| | | | GTAVHPEFAYRLRPGSGGSNGEGNSDDGLIHGLPSTLEK |
| | | | LDAPGVFEREDEHGMYTELRLPKDFGPGSVLVVRTQLVD |
| | | | FKPNLDWKIRTSADDAVARLDLAALNAALYRCDAEERDTI |
| | | | GEGVYDVPGLGPLPYCGLQGWHTHLRHIIPNNDLGHPLC |
| | | | GHLRQGTWALDYVVRRLHAYTEYHAPLAILAAWFEERWA |
| | | | LVKRVPNFLMPRYFALTVHTAYCALIRRALALMPGDVISSS |
| | | | RFTRELALTSVQLVGHVNSAALRPVCQDKARTSMSAGLA |
| | | | HFSTHHMRCWGRDVFIALEGLLLVTGRHQEAREHILAFGS |
| | | | TLKHGLIPNLLDSGRYPRYNARDATWFWLQAVQTYCNHA |
| | | | DNGLELLDEPVARRFPDGETFVEFDSDQAFSTTSTVAELI |
| | | | QEILQRHAQGIRFREWNAGPRLDEHMRDAGFDIEVSVDF |
| | | | ASTGFVSGGNQWNCGTWMDKMGSSDKAGIRGVPGTPR |
| | | | DGADIEIVGLQKSALRWVSSLHEMERFPFEGVTTSSGKRV |
| | | | TYAEWDALVQRSFEKHFWVPLSAADDAEYHVDTALVNRR |
| | | | GIYRDTYGSTQAWADYQFRPNIAVAMVVAPELFDTGHAL |
| | | | VCLHKMRAVLSGPLGMRTLDPSDMRYRPDYDNSNDSDD |
| | | | ALVAHGINYHQGPEWLWPTGYYLAAQLEFMRHAVNQRS |
| | | | TGGKATHALRTIFHELHAHMVALKHHIANAPYAGLPELTN |
| | | | RDGSYCRDSCETQAWSSACLLMALQRMMDLEQKVHRLI |
| | | | ELSDIVQ |
| ARBA000 | Linderina | 327 | MSVSRFSTPVEPEHPPFMVWNLDLNEDGTTPHDKSFIRIP |
| 20723 | pennispora | | SVLNQSCAVRFRILIGSTASIDAVLHTNYPLDGSDYVRTKF |
| (6X-His) | | | HSKRFNFNNSTELICEFRIQRPGPYQYYVTYKSVDDEESD |
| | | | YTDCISCDSDEVPTLERVYRVFKDRRTPTSYFLVDPQLTL |
| | | | GGQHLALDGIALQSVSPKWLGPMKGWSKHLANSSKMGY |
| | | | NMLHFIPMQQRGGSDSPYSLYNQLELSDDLFEGEKLSKE |
| | | | EKDQRLRTVCLEMHHKHHLLGITDMVWNHTAYNSEWLC |
| | | | DHPEAGFNLVNSPHLRSAYELDAKLCEFSKNLPEYGFDR |
| | | | MVSTVAQVDSLMEGVNTHVIQPLKLWEFYVVDVEGTVQA |
| | | | VSDAWDKAGVEMSADDIAKATELQGDERNEWLQTFVINN |
| | | | DPHTLSTRHGRTIDSERAVAVLRLLNGNDKKQGLEQLRRL |
| | | | LDLINLPYYREYDSDVARIRKNVGERVKYERLDRGSWKFG |
| | | | KPVDDDFKIVDPLFTTVTPRGGDSGAGKVNEDLYHLANN |
| | | | GWIWGGNPLDNFAGPQSKAYLQREVIVWGDCVKLNYGT |
| | | | QASDNPWLWEHMRQYTLSMAKAFHGFRIDNCHSTPIELA |
| | | | EYLLDQARKVKPDLYVIAELFTGSEATDQIFVQRLGINSLIR |
| | | | EAMQAWDSHEMSRLAHRHGGRPVGSLAVDCLGEPGFFT |
| | | | DDEHGGTRVDGIVAPLSASLPHAIFFDCTHDNEVPAQKRT |
| | | | MQDALPNAAIVAMTACATGSNRGYDELYPELLNIVHESRQ |
| | | | YAPLEDPLSVGLGEAKAKLNQLHAEMAQFQEVHVHHENE |
| | | | FVTVQRMHPVTRHGVLMVAHCAFRGASENSHFENPRLY |
| | | | GTAVHPEFAYRLRPGSGGSNGEGNSDDGLIHGLPSTLEK |
| | | | LDAPGVFEREDEHGMYTELRLPKDFGPGSVLVVRTQLVD |
| | | | FKPNLDWKIRTSADDAVARLDLAALNAALYRCDAEERDTI |
| | | | GEGVYDVPGLGPLPYCGLQGWHTHLRHIIPNNDLGHPLC |
| | | | GHLRQGTWALDYVVRRLHAYTEYHAPLAILAAWFEERWA |
| | | | LVKRVPNFLMPRYFALTVHTAYCALIRRALALMPGDVISSS |
| | | | RFTRELALTSVQLVGHVNSAALRPVCQDKARTSMSAGLA |
| | | | HFSTHHMRCWGRDVFIALEGLLLVTGRHQEAREHILAFGS |
| | | | TLKHGLIPNLLDSGRYPRYNARDATWFWLQAVQTYCNHA |
| | | | DNGLELLDEPVARRFPDGETFVEFDSDQAFSTTSTVAELI |
| | | | QEILQRHAQGIRFREWNAGPRLDEHMRDAGFDIEVSVDF |
| | | | ASTGFVSGGNQWNCGTWMDKMGSSDKAGIRGVPGTPR |
| | | | DGADIEIVGLQKSALRWVSSLHEMERFPFEGVTTSSGKRV |
| | | | TYAEWDALVQRSFEKHFWVPLSAADDAEYHVDTALVNRR |
| | | | GIYRDTYGSTQAWADYQFRPNIAVAMVVAPELFDTGHAL |
| | | | VCLHKMRAVLSGPLGMRTLDPSDMRYRPDYDNSNDSDD |
| | | | ALVAHGINYHQGPEWLWPTGYYLAAQLEFMRHAVNQRS |
| | | | TGGKATHALRTIFHELHAHMVALKHHIANAPYAGLPELTN |
| | | | RDGSYCRDSCETQAWSSACLLMALQRMMDLEQKVHRLI |
| | | | ELSDIVQHHHHHH |
| ARBA000 | Aspergillus | 328 | MSSPRQVYLLPLKDDGSPDVPGGYIYLPAPSDPSYLLRFV |
| 20723 | uvarum CBS | | IEGTSSICREGQLWVNIPEDGHPFDRSAFRSFRLSPDFNK |
| | 121591 | | NIQIDVPITSPGSFAFYVTFSPLPEFSVSQTATPEPTRTPTY |
| | | | YIDVSPKLTLRGRDLPLNALSIYSVISKFLGKYPTDWDKHL |
| | | | NGISQRNYNMIHFTPLMKRGDSNSPYSIFDQLQFDDAVFP |
| | | | NGEADVANLVTKMEEKHGLLSLTDVVWNHTANNSKWLEE |
| | | | HPEAGYSVETAPWLESALDLDNALLEIGENLASLGLPTEF |
| | | | KSVDDLVAVMNAIREQVIHKLKLWEFYVVNVAADTQKVIQ |
| | | | QWQTSKSIDITSDEWSQHKLKDFGNWTLEEQAIFVREKAI |
| | | | PTSKQLLGRYSRAVEPKFGAAILTVLLGPSDAASSDTTAA |
| | | | EKAISKLLDEVNLPFYKEYDADVAEIMNQLFNRIKYLRIDSH |
| | | | GPKLGPVTEQNPLIETYFTRLPLNDTTKKHDSKALALVNN |
| | | | GWIWNADALRDNAGPFSRSYLRREVIVWGDCVKLRYGSS |
| | | | PEDNPFLWEFMTKYTRLMAKYFSGFRIDNCHSTPLVVAEY |
| | | | LLDEARKVRPNLTVFAELFTGSEEADYIFVKRLGINALIREA |
| | | | MQAWSTGELSRLVHRHGGRPIGSFGVDLPSSGSSHAIAS |
| | | | SGIDGGKEKVSHIRPNPVQALFMDCTHDNEMPAQKRTAID |
| | | | TLPNAALVAMCASAIGSVIGYDEIYPKLVDLVHETRLYSSR |
| | | | FSESSKVDLDSLEGGIGGVKKLLNELHTIMGSEGYDETHIH |
| | | | HDGEYITVHRVHPKTRKGVELIAHTAFPGQDSKAILAPTRL |
| | | | VGTRAKHIGTWRLEVDSSESTRKEVSEDKSYLRGLPSQV |
| | | | HAIEGTKAEQDGNDTVISVLESLVAGSISLYETSIPSAEHAS |
| | | | GLDVHITEGVDEAFSNLGLVDLNFVLYRCEAEERDSSNGQ |
| | | | DGVYSIPNHGSLVYAGLQGWWSVLENVIKYNELGHPLCD |
| | | | HLRNGQWALDFIVGRLEKVAKTEGYSALHKPAAWLREKF |
| | | | DAVRSLPSFLLPRYFAIIVQTAYNAAWKRGIQLLGGNIQKG |
| | | | QEFIHQLSMVSVQMTGFVNSASLWPTKRVPSLAAGLPHF |
| | | | AVDWARCWGRDVFISLRGLLLCTSRFDDAREHIFAFASVL |
| | | | KHGMIPNLLSSGRLPRYNSRDSVWFFLQSIQDYTLMAPD |
| | | | GIKLLDEKVPRRFLPYDDTWFPFDDPRAYSKHSTISEVIQE |
| | | | VFQRHARGLSFREYNAGPDLDVQMKPEGFEINVQVDWD |
| | | | TGLIFGGSQFNCGTWQDKMGESEKAKNKGVPGTPRDGA |
| | | | AIEITGLVYSALNWVAKLHEQGLYQHSGVDLGEGKSVTFK |
| | | | SWAAKIKENFERCYYIPSNPEEDASYVVDRNVVNRRGIYK |
| | | | DLYKSGKPYEDYQLRSNFPIAMTVAPDLFTPSKALSALALA |
| | | | DEVLVGPVGMATLDPSDLNYRPNYNNSEDSEDFATSKGR |
| | | | NYHQGPEWVWQRGFFLRAFLHFDLARRATREERTEAYQ |
| | | | QVTRRLEGCKKALRESPWRGLTELSNKDGAYCADSSPTQ |
| | | | AWSAGCLLDLYYDASQYTQDDSAVQNEVDMRPTVLKSS |
| | | | ACPYALIIQDIRQTHPSAEHNLTRVMSLSQSAAAAMLTAVD |
| | | | APIEVHEPSWGDQTNPRDFLTQFVISMTIGLGAFLAFCVLR |
| | | | PKWTELYAARRRQRCAASHLPELPDSFFGWIPVLYRITEE |
| | | | QVLESAGLDAFVFLTFLKFAIRFLSGVFFFALVIILPMHYRN |
| | | | TGKSGVPGWDDDDEPVDVFDGDKDKKKIISDPNYLWMYV |
| | | | VFTYIFTGLAVYMLVQDTNKVIRIRQQYLGSQTSTTDRTIRL |
| | | | SGIPHDMGSEEKIREFIEGLQIGKVQSVTLCKDWRELDRLI |
| | | | DERLKILRNLEWAWTKHLGYKRVKANANRLPLLQQHARG |
| | | | SSIVSEEDSERIQLISDSTPAHVLDYAHKRPTTRIWYGPFK |
| | | | LRYRNVDAIDYYEEKLRRIDEQIQLARQKEYPPTEIAFVTM |
| | | | ESIAASQMVVQAILDPQPMQLLARLAPAPADVVWKNTYIP |
| | | | RSRRMMQSWSITVVIGFLTVFWSVLLIPVAYLLEYETLHKV |
| | | | FPQLADALSRNPLAKSLVQTGLPTLVLSLLTIAVPYLYSWL |
| | | | SHQQGMMSRGDIELSVISKTFFFSFFNLFLVFTVFGTATTF |
| | | | FGFWENLRDAFKDATTIAFALAKTLENFAPFYINFLCLQGI |
| | | | GLLPFRLLEFGSVAMYPINFLTAKTPRDYAELNTPPVFSYG |
| | | | YSIPQTILSLIICVVYSVFPSSWLICLFGLIYFTIGKFIYKYQLL |
| | | | YAMDHQQHSTGRAWPMICSRVLMGLIVFQLAMIGVLALR |
| | | | RAITRSLLIVPLLMATVWFSYFFSRNYEPLMKFIALKSIDRE |
| | | | RPGGGDISPSPSSTFSPPSGLERDAFPIRIGGQELGLRLK |
| | | | KYVNPSLILPLHDAWLPGRSTAPGYQHDSGVEETPNVAA |
| | | | EESV |
| ARBA000 | Aspergillus | 329 | MSSPRQVYLLPLKDDGSPDVPGGYIYLPAPSDPSYLLRFV |
| 20723 | uvarum CBS | | IEGTSSICREGQLWVNIPEDGHPFDRSAFRSFRLSPDFNK |
| (6X-His) | 121591 | | NIQIDVPITSPGSFAFYVTFSPLPEFSVSQTATPEPTRTPTY |
| | | | YIDVSPKLTLRGRDLPLNALSIYSVISKFLGKYPTDWDKHL |
| | | | NGISQRNYNMIHFTPLMKRGDSNSPYSIFDQLQFDDAVFP |
| | | | NGEADVANLVTKMEEKHGLLSLTDVVWNHTANNSKWLEE |
| | | | HPEAGYSVETAPWLESALDLDNALLEIGENLASLGLPTEF |
| | | | KSVDDLVAVMNAIREQVIHKLKLWEFYVVNVAADTQKVIQ |
| | | | QWQTSKSIDITSDEWSQHKLKDFGNWTLEEQAIFVREKAI |
| | | | PTSKQLLGRYSRAVEPKFGAAILTVLLGPSDAASSDTTAA |
| | | | EKAISKLLDEVNLPFYKEYDADVAEIMNQLFNRIKYLRIDSH |
| | | | GPKLGPVTEQNPLIETYFTRLPLNDTTKKHDSKALALVNN |
| | | | GWIWNADALRDNAGPFSRSYLRREVIVWGDCVKLRYGSS |
| | | | PEDNPFLWEFMTKYTRLMAKYFSGFRIDNCHSTPLVVAEY |
| | | | LLDEARKVRPNLTVFAELFTGSEEADYIFVKRLGINALIREA |
| | | | MQAWSTGELSRLVHRHGGRPIGSFGVDLPSSGSSHAIAS |
| | | | SGIDGGKEKVSHIRPNPVQALFMDCTHDNEMPAQKRTAID |
| | | | TLPNAALVAMCASAIGSVIGYDEIYPKLVDLVHETRLYSSR |
| | | | FSESSKVDLDSLEGGIGGVKKLLNELHTIMGSEGYDETHIH |
| | | | HDGEYITVHRVHPKTRKGVFLIAHTAFPGQDSKAILAPTRL |
| | | | VGTRAKHIGTWRLEVDSSESTRKEVSEDKSYLRGLPSQV |
| | | | HAIEGTKAEQDGNDTVISVLESLVAGSISLYETSIPSAEHAS |
| | | | GLDVHITEGVDEAFSNLGLVDLNFVLYRCEAEERDSSNGQ |
| | | | DGVYSIPNHGSLVYAGLQGWWSVLENVIKYNELGHPLCD |
| | | | HLRNGQWALDFIVGRLEKVAKTEGYSALHKPAAWLREKF |
| | | | DAVRSLPSFLLPRYFAIIVQTAYNAAWKRGIQLLGGNIQKG |
| | | | QEFIHQLSMVSVQMTGFVNSASLWPTKRVPSLAAGLPHF |
| | | | AVDWARCWGRDVFISLRGLLLCTSRFDDAREHIFAFASVL |
| | | | KHGMIPNLLSSGRLPRYNSRDSVWFFLQSIQDYTLMAPD |
| | | | GIKLLDEKVPRRFLPYDDTWFPFDDPRAYSKHSTISEVIQE |
| | | | VFQRHARGLSFREYNAGPDLDVQMKPEGFEINVQVDWD |
| | | | TGLIFGGSQFNCGTWQDKMGESEKAKNKGVPGTPRDGA |
| | | | AIEITGLVYSALNWVAKLHEQGLYQHSGVDLGEGKSVTFK |
| | | | SWAAKIKENFERCYYIPSNPEEDASYVVDRNVVNRRGIYK |
| | | | DLYKSGKPYEDYQLRSNFPIAMTVAPDLFTPSKALSALALA |
| | | | DEVLVGPVGMATLDPSDLNYRPNYNNSEDSEDFATSKGR |
| | | | NYHQGPEWVWQRGFFLRAFLHFDLARRATREERTEAYQ |
| | | | QVTRRLEGCKKALRESPWRGLTELSNKDGAYCADSSPTQ |
| | | | AWSAGCLLDLYYDASQYTQDDSAVQNEVDMRPTVLKSS |
| | | | ACPYALIIQDIRQTHPSAEHNLTRVMSLSQSAAAAMLTAVD |
| | | | APIEVHEPSWGDQTNPRDFLTQFVISMTIGLGAFLAFCVLR |
| | | | PKWTELYAARRRQRCAASHLPELPDSFFGWIPVLYRITEE |
| | | | QVLESAGLDAFVFLTFLKFAIRFLSGVFFFALVIILPMHYRN |
| | | | TGKSGVPGWDDDDEPVDVFDGDKDKKKIISDPNYLWMYV |
| | | | VFTYIFTGLAVYMLVQDTNKVIRIRQQYLGSQTSTTDRTIRL |
| | | | SGIPHDMGSEEKIREFIEGLQIGKVQSVTLCKDWRELDRLI |
| | | | DERLKILRNLEWAWTKHLGYKRVKANANRLPLLQQHARG |
| | | | SSIVSEEDSERIQLISDSTPAHVLDYAHKRPTTRIWYGPFK |
| | | | LRYRNVDAIDYYEEKLRRIDEQIQLARQKEYPPTEIAFVTM |
| | | | ESIAASQMVVQAILDPQPMQLLARLAPAPADVVWKNTYIP |
| | | | RSRRMMQSWSITVVIGFLTVFWSVLLIPVAYLLEYETLHKV |
| | | | FPQLADALSRNPLAKSLVQTGLPTLVLSLLTIAVPYLYSWL |
| | | | SHQQGMMSRGDIELSVISKTFFFSFFNLFLVFTVFGTATTF |
| | | | FGFWENLRDAFKDATTIAFALAKTLENFAPFYINFLCLQGI |
| | | | GLLPFRLLEFGSVAMYPINFLTAKTPRDYAELNTPPVFSYG |
| | | | YSIPQTILSLIICVVYSVFPSSWLICLFGLIYFTIGKFIYKYQLL |
| | | | YAMDHQQHSTGRAWPMICSRVLMGLIVFQLAMIGVLALR |
| | | | RAITRSLLIVPLLMATVWFSYFFSRNYEPLMKFIALKSIDRE |
| | | | RPGGGDISPSPSSTFSPPSGLERDAFPIRIGGQELGLRLK |
| | | | KYVNPSLILPLHDAWLPGRSTAPGYQHDSGVEETPNVAA |
| | | | EESVHHHHHH |
| ARBA000 | Candida auris | 330 | MRSVLLRLDDEGSPIKNDAVHNGVFIFPSYAPPEGWTRG |
| 20723 | | | APLFELVVQLNAAAPVSKKGILCTNVPKDGKTAFQRSNFQ |
| | | | RYPLSTSFTKDTTIRVPIYSPGPYNLYIEYENEEQEKVKTP |
| | | | DHYFVVPPNLMVNDKYVPFNAINIQTVVSKWAGPLDNWD |
| | | | KLFRYINEKGYDMIHFTPLQERGSSNSPYSIRDQLRFDPNL |
| | | | FKDTESAISFIHRTTEKHNLLSLTDVVWNHTSDDSPWLRD |
| | | | HPDAGYNAETAPHLTSAIELDKALLEYSDKLADLGLPTELK |
| | | | SESDLKKLVDGIQDHVLGPLELWQYYVFDKKSSLEDLRKA |
| | | | HEIGGVSPSTIPDDTDVSDVKQLASFILKRASIHEKPILGDR |
| | | | FSNKLDPNKFLSYLLTLAGDDFNEIEKKASQIIDEINVSLYA |
| | | | TFDDDIRSIKAQIADRARYLRLADNGPKLGPITKENRFTES |
| | | | YFTRFEGKDGKEYALANNGWIWGGNPLVDFASSESRAYI |
| | | | RREVIVWSDCVKLRYGKGPEDSPHVWKRMIEYTKNSAKA |
| | | | FSGFRLDNCHSTPLHVGEALLDAARSVNPDLYVVAELFTG |
| | | | SEEMDKIFVERLGINSLIREAMQAWSVEELSRLVHKHGGR |
| | | | PIGSLTWMPLDDFSFPCNKEPTAGKYSEGHSELEIPHVLA |
| | | | KQAPHALFMDCTHDNQTPAQVRTVGDTLSTAALVAFCSS |
| | | | AIGTTFGFDETYPELLDIVNETRTYDYDFKTGIAEARRKMN |
| | | | DVRDSLAAESKDAASDQEMYIHHEGQYITIQRYNTVTGEG |
| | | | WFLIARTKFTDSPGNQILSPVILKGTNVKYEFGYTLKKVGE |
| | | | YSKDDKYLTSIPTKLEEVSLPPVEHNGDEAIIRVDNNFVPG |
| | | | SIAVFSTKIANVDASLDSYVKKGAVEASLGLDLYDLNAILYR |
| | | | CEPEERDASNGMEGVYNIPNYGPLVYAGLQGWVSVLKQ |
| | | | AVWRNDLAHPLCDHLREGFWAADYIVNRLEKYSKNSKNL |
| | | | HKFHAWLKSRVDAIRDVPYFLRPHYFALVVGVAYEAARFR |
| | | | VLRQLDDHIKTATNFVQSLALTSVQMCGYMNNTSLLPDKN |
| | | | IPCLAAGLPHFSNDYMRCWGRDVFISYRGLLIVPGRHEDA |
| | | | KQHILGFAKTLKHGLIPNLLDAGRNPRYNARDAAWFFARA |
| | | | VEEYVTHVPHGEQILDETVSRRFPLDDTYVSWDDKRAFS |
| | | | YETSIRDILYEILSRHAKGIKYREANAGPNLDSQMKDEGFN |
| | | | VEVYVDWNTGLVHGGSQWNCGTWMDKMGESEKAGNK |
| | | | GVPGTPRDGAAVELQGLLKSTLRFVNGLHKKGLFEHTEV |
| | | | EKPDGSKISLIEWESLIQENFEKCFFVPEDPAEDDHYEINP |
| | | | DWVNRRGIYKDLFKSGKEYEDYQLRANFPIAMCVAPELFT |
| | | | PKNALSAINKADEIIRGPVGMRTLDPSDWNYRPNYNNSED |
| | | | SEDFATSKGRNYHQGPEWVWLFGYFIRAFLYFNHFADNG |
| | | | GEGSPTKELLTEVNKRISGHKKWIRESPWAGITELTNQNG |
| | | | ALCHDSSPTQAWTSSLMLRNLSKSAARMAKKPAPLFGRS |
| | | | LSTLTLNTTTDSYPTNHFEKGTEPYLTPSFIDNQLVKSESQ |
| | | | TWFDIHDPATNEVISKVPQSTPQELDDAIASAEKAFHEWK |
| | | | NYSIIKRQGIAFKFVELLKKNHDRLAGMIVLEQGKTFPDAK |
| | | | GDVLRGLQVAEAACNAPNDLMASSLEVATDMETKMVREP |
| | | | LGVIGSICPFNFPAMVPLWSLPLVLVTGNTAVIKPSERVPG |
| | | | AAMIIAQLAAEAGVPPGVLNIVHGKHDTVNKIIEDPRVKAIN |
| | | | FVGGDKAGKYIYEKGTSLGKRVQANLGAKNHLIVLPDADK |
| | | | NHFINAVNGAAFGAAGQRCMAISVLVTVGEKTKQWVEEV |
| | | | ASDAAKLVTGSGFDPKSDLGPLVNPESLTRAKEIINDSEK |
| | | | QGAKVILDGRNFKPSDPKFAKGNFLGPTILTNVKPGMRAY |
| | | | DEEIFAPVLSVVNVDTFDEAIALVNSNRYGNGVSLFTSAGA |
| | | | AAQKFTKEIACGQVGVNVPIPVPLPMFSFTGNKGSFLGDL |
| | | | NFYGKAGVTFLTQPKTITTLWRTSTVDQVLAPSTAMPTQH |
| ARBA000 | Candida auris | 331 | MRSVLLRLDDEGSPIKNDAVHNGVFIFPSYAPPEGWTRG |
| 20723 | | | APLFELVVQLNAAAPVSKKGILCTNVPKDGKTAFQRSNFQ |
| (6X-His) | | | RYPLSTSFTKDTTIRVPIYSPGPYNLYIEYENEEQEKVKTP |
| | | | DHYFVVPPNLMVNDKYVPFNAINIQTVVSKWAGPLDNWD |
| | | | KLFRYINEKGYDMIHFTPLQERGSSNSPYSIRDQLRFDPNL |
| | | | FKDTESAISFIHRTTEKHNLLSLTDVVWNHTSDDSPWLRD |
| | | | HPDAGYNAETAPHLTSAIELDKALLEYSDKLADLGLPTELK |
| | | | SESDLKKLVDGIQDHVLGPLELWQYYVFDKKSSLEDLRKA |
| | | | HEIGGVSPSTIPDDTDVSDVKQLASFILKRASIHEKPILGDR |
| | | | FSNKLDPNKFLSYLLTLAGDDFNEIEKKASQIIDEINVSLYA |
| | | | TFDDDIRSIKAQIADRARYLRLADNGPKLGPITKENRFTES |
| | | | YFTRFEGKDGKEYALANNGWIWGGNPLVDFASSESRAYI |
| | | | RREVIVWSDCVKLRYGKGPEDSPHVWKRMIEYTKNSAKA |
| | | | FSGFRLDNCHSTPLHVGEALLDAARSVNPDLYVVAELFTG |
| | | | SEEMDKIFVERLGINSLIREAMQAWSVEELSRLVHKHGGR |
| | | | PIGSLTWMPLDDFSFPCNKEPTAGKYSEGHSELEIPHVLA |
| | | | KQAPHALFMDCTHDNQTPAQVRTVGDTLSTAALVAFCSS |
| | | | AIGTTFGFDETYPELLDIVNETRTYDYDFKTGIAEARRKMN |
| | | | DVRDSLAAESKDAASDQEMYIHHEGQYITIQRYNTVTGEG |
| | | | WFLIARTKFTDSPGNQILSPVILKGTNVKYEFGYTLKKVGE |
| | | | YSKDDKYLTSIPTKLEEVSLPPVEHNGDEAIIRVDNNFVPG |
| | | | SIAVFSTKIANVDASLDSYVKKGAVEASLGLDLYDLNAILYR |
| | | | CEPEERDASNGMEGVYNIPNYGPLVYAGLQGWVSVLKQ |
| | | | AVWRNDLAHPLCDHLREGFWAADYIVNRLEKYSKNSKNL |
| | | | HKFHAWLKSRVDAIRDVPYFLRPHYFALVVGVAYEAARFR |
| | | | VLRQLDDHIKTATNFVQSLALTSVQMCGYMNNTSLLPDKN |
| | | | IPCLAAGLPHFSNDYMRCWGRDVFISYRGLLIVPGRHEDA |
| | | | KQHILGFAKTLKHGLIPNLLDAGRNPRYNARDAAWFFARA |
| | | | VEEYVTHVPHGEQILDETVSRRFPLDDTYVSWDDKRAFS |
| | | | YETSIRDILYEILSRHAKGIKYREANAGPNLDSQMKDEGFN |
| | | | VEVYVDWNTGLVHGGSQWNCGTWMDKMGESEKAGNK |
| | | | GVPGTPRDGAAVELQGLLKSTLRFVNGLHKKGLFEHTEV |
| | | | EKPDGSKISLIEWESLIQENFEKCFFVPEDPAEDDHYEINP |
| | | | DVVNRRGIYKDLFKSGKEYEDYQLRANFPIAMCVAPELFT |
| | | | PKNALSAINKADEIIRGPVGMRTLDPSDWNYRPNYNNSED |
| | | | SEDFATSKGRNYHQGPEWVWLFGYFIRAFLYFNHFADNG |
| | | | GEGSPTKELLTEVNKRISGHKKWIRESPWAGITELTNQNG |
| | | | ALCHDSSPTQAWTSSLMLRNLSKSAARMAKKPAPLFGRS |
| | | | LSTLTLNTTTDSYPTNHFEKGTEPYLTPSFIDNQLVKSESQ |
| | | | TWFDIHDPATNEVISKVPQSTPQELDDAIASAEKAFHEWK |
| | | | NYSIIKRQGIAFKFVELLKKNHDRLAGMIVLEQGKTFPDAK |
| | | | GDVLRGLQVAEAACNAPNDLMASSLEVATDMETKMVREP |
| | | | LGVIGSICPFNFPAMVPLWSLPLVLVTGNTAVIKPSERVPG |
| | | | AAMIIAQLAAEAGVPPGVLNIVHGKHDTVNKIIEDPRVKAIN |
| | | | FVGGDKAGKYIYEKGTSLGKRVQANLGAKNHLIVLPDADK |
| | | | NHFINAVNGAAFGAAGQRCMAISVLVTVGEKTKQWVEEV |
| | | | ASDAAKLVTGSGFDPKSDLGPLVNPESLTRAKEIINDSEK |
| | | | QGAKVILDGRNFKPSDPKFAKGNFLGPTILTNVKPGMRAY |
| | | | DEEIFAPVLSVVNVDTFDEAIALVNSNRYGNGVSLFTSAGA |
| | | | AAQKFTKEIACGQVGVNVPIPVPLPMFSFTGNKGSFLGDL |
| | | | NFYGKAGVTFLTQPKTITTLWRTSTVDQVLAPSTAMPTQH |
| | | | HHHHHH |
| ARBA000 | Pneumocystis sp. | 332 | MWSGFSRHFLYGYKAKIGYTGVQTVMLTISTFTSLSSIFLY |
| 20723 | ′macacae′ | | YHIYGNPIKAMTPEEHGLHPPKYPWPHKGFLSSYDHKSLR |
| | | | RGYQVYKEVCSACHSLNLVAWRNLVGVTHTLDEAKAMAE |
| | | | EYEYEDGPDESVLRLIIGKLFDYMPKPYPNEEAARAANAG |
| | | | AFPPDLSLIIKARHGGCDYVFSLLTGYVDPPAGVVLADGM |
| | | | NYNPYFPNGQIAMARLLYDGLIEYEDGTPATTSQMAKDVV |
| | | | SFLNWAAEPEHDDRKRMGFQTLIILSTLFALNLWVKRFKW |
| | | | APIKTSLFCMLYEFTKYLPFYRRLSSSEFKSVMAEIQIYSLK |
| | | | LSGTGSPLISGKYISLPPPFVPYYLRFEIDEITFIYYKSVLKH |
| | | | NFPPKGSKFNRDKWYSIPLDIDFSSIKNIDILVHTAGLFSYKI |
| | | | EYFQLPLWGLEEFDHSVPRKSTDEFYFTVLPALKINGSYIS |
| | | | LNSLVIQSVVSKWMGPISEWDEIMHYIGKKKCYNMVHFTP |
| | | | LQSLGESNSPYSIYDQLSFSDDLFDVKLESKQKNNIVDKYL |
| | | | RKMEIEWGLLSITDVVWNHTARDSKWLQDHPEVGYNLHN |
| | | | SPHLIAAYELDASLLEFSANLGELGYPTNPKNIDDLLKIIDGI |
| | | | KNTVLEDLRLWEFYVVNVKESVKYTIDAYKKKKIVELDDKF |
| | | | DININWSLKEKVNAFIGVSAITNYNVLGERFAKRLVPEIGAA |
| | | | ILYKLFGIDIEEQVLDDELTKILNEFNLEYYIEYDIDKKIIIDQIY |
| | | | NRSKYLRLDDNGPKKGEISRKNPLVETYFSLIERNKNTLLY |
| | | | SDRSLCLANNGWVWNSSLNDFAGQESKAYLLRQVIVWG |
| | | | DCVKLRYGKDPSDNPYLWDHMTKYTQMLARLFNGFRIDN |
| | | | CHSTPLHVGIYLLDKAREIRNDLYIVAELFTGNEEMDLVFM |
| | | | QKLGISSLIREAMQAWCPGELSRFIYKYGGKPIGSLDSISS |
| | | | KYHYLTSIKNCKFSNVVSVQNSQIHTLFMDCTHDNQMPA |
| | | | QKRTAYDTLPNAALVSMCNCSIGSVMGYDEIVPHYINIVM |
| | | | ESRKYYYSLENVIGIESVKSVLNKIHVEMGKDNFEEIYVHH |
| | | | ENSYITVHTVNPKNYHGYFLLAHTAFLMHENNKIRPIVLRG |
| | | | TKVDVVFSLSLEIESFEDSKDPDFIKGLSSTIIELDSPIITTGE |
| | | | DSEGFFTCIEVPKKFPPGSIFLLKTWIENVEDLDNFICTDLD |
| | | | NIMENFDLVDLNLIMYRCDQEERNTINDGCYNIPGFGDLV |
| | | | YCGFEGWMPVLKEIIRNNILDHPLCQHLRQGQWALDYVV |
| | | | QRLYKMENILSRLRQLADWLQMRFSRIKKIPNFLLPRYFIT |
| | | | TLYLTSMAARNRAIGLMSPHIQNGHTFIKSLSLCSVQMHGI |
| | | | VKSASLDPFINKPCLAAGLPHFSYSWKRCWGRDVFISLRG |
| | | | LFLVTGRFDDAKRHILAFASVLKHGMVPNLLDSLKRPRYN |
| | | | SRDSVWFFLQAIQEYTKLVPDGIQILYERVKRRFPLDDSFV |
| | | | ELDDYRAYSHESTILEVIHEIMQRHALGLHFREANAGLGLD |
| | | | TEMTDKGFDIDIYVDWKTGLIFGGSKYNCGTWMDKMGES |
| | | | LKAGNKGVPGTPRDGAAIEISGMLKSTLRWINELRRKNIYK |
| | | | WHCVNIKENGEFKDVTFEEWEKKIQNSFEFCYYIPFDPQE |
| | | | DSKYDVNISMINRRGIYKDVYRSNKEYEDYQLRPNFAIAM |
| | | | WVAPELFTFGYAVRAIEIADKVIRGPMGMLTLDPSDKDYRP |
| | | | YYINSYDSDDFATSKGRNYHQGPEWLWCTGYFLRAFLYF |
| | | | SRFYYNFTKNKDIVCYHVHNRLVTLMKEIEENPWAGLVEL |
| | | | TNKNGEFCNDSNNTQSWSASTILDLYDDILKLRTLDKS |
| ARBA000 | Pneumocystis sp. | 333 | MWSGFSRHFLYGYKAKIGYTGVQTVMLTISTFTSLSSIFLY |
| 20723 | ′macacae′ | | YHIYGNPIKAMTPEEHGLHPPKYPWPHKGFLSSYDHKSLR |
| (6X-His) | | | RGYQVYKEVCSACHSLNLVAWRNLVGVTHTLDEAKAMAE |
| | | | EYEYEDGPDESVLRLIIGKLFDYMPKPYPNEEAARAANAG |
| | | | AFPPDLSLIIKARHGGCDYVFSLLTGYVDPPAGVVLADGM |
| | | | NYNPYFPNGQIAMARLLYDGLIEYEDGTPATTSQMAKDVV |
| | | | SFLNWAAEPEHDDRKRMGFQTLIILSTLFALNLWVKRFKW |
| | | | APIKTSLFCMLYEFTKYLPFYRRLSSSEFKSVMAEIQIYSLK |
| | | | LSGTGSPLISGKYISLPPPFVPYYLRFEIDEITFIYYKSVLKH |
| | | | NFPPKGSKFNRDKWYSIPLDIDFSSIKNIDILVHTAGLFSYKI |
| | | | EYFQLPLWGLEEFDHSVPRKSTDEFYFTVLPALKINGSYIS |
| | | | LNSLVIQSVVSKWMGPISEWDEIMHYIGKKKCYNMVHFTP |
| | | | LQSLGESNSPYSIYDQLSFSDDLFDVKLESKQKNNIVDKYL |
| | | | RKMEIEWGLLSITDVVWNHTARDSKWLQDHPEVGYNLHN |
| | | | SPHLIAAYELDASLLEFSANLGELGYPTNPKNIDDLLKIIDGI |
| | | | KNTVLEDLRLWEFYVVNVKESVKYTIDAYKKKKIVELDDKF |
| | | | DININWSLKEKVNAFIGVSAITNYNVLGERFAKRLVPEIGAA |
| | | | ILYKLFGIDIEEQVLDDELTKILNEFNLEYYIEYDIDKKIIIDQIY |
| | | | NRSKYLRLDDNGPKKGEISRKNPLVETYFSLIERNKNTLLY |
| | | | SDRSLCLANNGWVWNSSLNDFAGQESKAYLLRQVIVWG |
| | | | DCVKLRYGKDPSDNPYLWDHMTKYTQMLARLFNGFRIDN |
| | | | CHSTPLHVGIYLLDKAREIRNDLYIVAELFTGNEEMDLVFM |
| | | | QKLGISSLIREAMQAWCPGELSRFIYKYGGKPIGSLDSISS |
| | | | KYHYLTSIKNCKFSNVVSVQNSQIHTLFMDCTHDNQMPA |
| | | | QKRTAYDTLPNAALVSMCNCSIGSVMGYDEIVPHYINIVM |
| | | | ESRKYYYSLENVIGIESVKSVLNKIHVEMGKDNFEEIYVHH |
| | | | ENSYITVHTVNPKNYHGYFLLAHTAFLMHENNKIRPIVLRG |
| | | | TKVDVVFSLSLEIESFEDSKDPDFIKGLSSTIIELDSPIITTGE |
| | | | DSEGFFTCIEVPKKFPPGSIFLLKTWIENVEDLDNFICTDLD |
| | | | NIMENFDLVDLNLIMYRCDQEERNTINDGCYNIPGFGDLV |
| | | | YCGFEGWMPVLKEIIRNNILDHPLCQHLRQGQWALDYVV |
| | | | QRLYKMENILSRLRQLADWLQMRFSRIKKIPNFLLPRYFIT |
| | | | TLYLTSMAARNRAIGLMSPHIQNGHTFIKSLSLCSVQMHGI |
| | | | VKSASLDPFINKPCLAAGLPHFSYSWKRCWGRDVFISLRG |
| | | | LFLVTGRFDDAKRHILAFASVLKHGMVPNLLDSLKRPRYN |
| | | | SRDSVWFFLQAIQEYTKLVPDGIQILYERVKRRFPLDDSFV |
| | | | ELDDYRAYSHESTILEVIHEIMQRHALGLHFREANAGLGLD |
| | | | TEMTDKGFDIDIYVDWKTGLIFGGSKYNCGTWMDKMGES |
| | | | LKAGNKGVPGTPRDGAAIEISGMLKSTLRWINELRRKNIYK |
| | | | WHCVNIKENGEFKDVTFEEWEKKIQNSFEFCYYIPFDPQE |
| | | | DSKYDVNISMINRRGIYKDVYRSNKEYEDYQLRPNFAIAM |
| | | | WAPELFTFGYAVRAIEIADKVIRGPMGMLTLDPSDKDYRP |
| | | | YYINSYDSDDFATSKGRNYHQGPEWLWCTGYFLRAFLYF |
| | | | SRFYYNFTKNKDIVCYHVHNRLVTLMKEIEENPWAGLVEL |
| | | | TNKNGEFCNDSNNTQSWSASTILDLYDDILKLRTLDKSHH |
| | | | HHHH |
| CCE64830.1 | Tetrapisispora | 334 | MKGRSQLENTLLLRLDSDGVPIANNTLYGSGVLTLPSLPY |
| | phaffii (strain | | KLEYINDDNPIVTLKIMIAAGSTISRDGLIWTNVPESSNVEF |
| | ATCC 24235/ | | NRDHFRKIIIKSSIHSDCYIDLKLFRPGSFCFYVSYRNKKNL |
| | CBS 4417/ | | LKTTKKHYFILGPSLFINDTYLPLNSINCQTIISKWLPKSTSP |
| | NBRC 1672/ | | EDNWQKIFKLIANKKYNMIHFTPLQQRGESNSPYSIYDQL |
| | NRRL Y-8282/ | | MFDPDFFKENQKSVKLLIDDLEKTHNMLSLTDIVFNHTANN |
| | UCD 70-5) | | SFWLRKHPESGYNQFTAPHLNAAIELDKALLDFSSRLKSL |
| | | | NYPVDLKNDNDLLEILDGIKVHVLGQLKLWEYYVINIKETV |
| | | | NEVEAKFDEISITNGDEEDESLNDEILNNLENLAAYVRENY |
| | | | NVGNFDNLAERYSHKLNIEKLIILLKKSKKFEKFDEETNTFII |
| | | | NLLDEINLPLYRIYDDDVNEILEQLFNRIKYLRISDDGPKLG |
| | | | VITNKSPLTEPYFTRFVGENDGIEYALANNGWIWNGNPLID |
| | | | FASKNSKAYLRREVIVWGDCVKLRYGKEPADSPYLWKRM |
| | | | SDYITMNAKIFHGFRIDNCHSTPLHVGEYFLDLARSHNPNL |
| | | | YVVAELFSGSETTDCLFVERLGISSLIREAMQASSEDELSN |
| | | | LIHRYGGRPIGSYKFVPMDTFAFSPDLELDEDYCTYSIKDK |
| | | | SIHCVSEIMIPRVLTCQPPHALFMDCTHDNKMPYEKRTIED |
| | | | TLPNAALVAFCSSAIGSVYGYDEIYPELLDLVNEKRKYDLS |
| | | | GSINKNLNDEVGICKIKRKLNAIRKEIAISSIDIEDSEMHVHH |
| | | | EGQYITFHRTNSKTGKGYYLIVRTKFSDFNEDQMFPSVTL |
| | | | RATKCKFNCAYSLEKVDTSFSPDNDLLTGVKSKIVELKDFT |
| | | | IHVDADSNSVIELPENFPPGSIAIFETGYVGIDDTLDHLIRS |
| | | | GALKATSNLNFESLNAVLFRSESEELDVSGNTFGAYNIPN |
| | | | HGKLVYCGLQGWVSVLRDIMINNDLAHPLSVNLREGHWA |
| | | | LDYILERLNLYNDKPGIRDVQAWLAERFDKIKQLPNFLVPS |
| | | | FFALTIGILYGCCRFKAMLLFPKYIGNANVFVQSLALTSIQM |
| | | | VSEVKSTSIFPNINVPCMCAGLPHFSTNYMRCWGRDVFIS |
| | | | FRGLLLVTGRYETAKQHILGFAKTTKHGLIPNLLDAGRNPR |
| | | | YNARDAAWFFLQAIQDYITIVPEGHSILSEKVSRRFPLNDK |
| | | | YITYEDPEAFSYSSSIEEIIYEILSRHAKGIKFREANAGPNLD |
| | | | RVMKDEGFNIEIAVDWSTGLIHGGNQFNCGTWMDKNGES |
| | | | EKAKSKGVPGTPRDGAAVEINGLLKSTLRFVLKLISEGKFK |
| | | | KTDVTRQDGSKISFADWNDLLQENFEKNYYIPLDPKDDNE |
| | | | YNTDSNIVNRRGIYKDLCGSGKPFEDYQLRPNQFIAMAVA |
| | | | PELFSPEHAMSAIKVADENIRGKLGMRTLDPSDYNYRPYY |
| | | | NNSEDSDDFATSKGRNYHQGPEWVWCYGYFMRSFHTF |
| | | | NFLANPLSRNETENRPSSYLMQQIYLRLDDHRKAIIESPW |
| | | | AGLPELTNKDGEFCQDSSPTQAWSSGCILDVLYDVWVAF |
| | | | DSKWYAKQIKQNNP |
| CCE64830.1 | Tetrapisispora | 335 | MKGRSQLENTLLLRLDSDGVPIANNTLYGSGVLTLPSLPY |
| (6X-His) | phaffii (strain | | KLEYINDDNPIVTLKIMIAAGSTISRDGLIWTNVPESSNVEF |
| | ATCC 24235/ | | NRDHFRKIIIKSSIHSDCYIDLKLFRPGSFCFYVSYRNKKNL |
| | CBS 4417/ | | LKTTKKHYFILGPSLFINDTYLPLNSINCQTIISKWLPKSTSP |
| | NBRC 1672/ | | EDNWQKIFKLIANKKYNMIHFTPLQQRGESNSPYSIYDQL |
| | NRRL Y-8282/ | | MFDPDFFKENQKSVKLLIDDLEKTHNMLSLTDIVFNHTANN |
| | UCD 70-5) | | SFWLRKHPESGYNQFTAPHLNAAIELDKALLDFSSRLKSL |
| | | | NYPVDLKNDNDLLEILDGIKVHVLGQLKLWEYYVINIKETV |
| | | | NEVEAKFDEISITNGDEEDESLNDEILNNLENLAAYVRENY |
| | | | NVGNFDNLAERYSHKLNIEKLIILLKKSKKFEKFDEETNTFII |
| | | | NLLDEINLPLYRIYDDDVNEILEQLFNRIKYLRISDDGPKLG |
| | | | VITNKSPLTEPYFTRFVGENDGIEYALANNGWIWNGNPLID |
| | | | FASKNSKAYLRREVIVWGDCVKLRYGKEPADSPYLWKRM |
| | | | SDYITMNAKIFHGFRIDNCHSTPLHVGEYFLDLARSHNPNL |
| | | | YVVAELFSGSETTDCLFVERLGISSLIREAMQASSEDELSN |
| | | | LIHRYGGRPIGSYKFVPMDTFAFSPDLELDEDYCTYSIKDK |
| | | | SIHCVSEIMIPRVLTCQPPHALFMDCTHDNKMPYEKRTIED |
| | | | TLPNAALVAFCSSAIGSVYGYDEIYPELLDLVNEKRKYDLS |
| | | | GSINKNLNDEVGICKIKRKLNAIRKEIAISSIDIEDSEMHVHH |
| | | | EGQYITFHRTNSKTGKGYYLIVRTKFSDFNEDQMFPSVTL |
| | | | RATKCKFNCAYSLEKVDTSFSPDNDLLTGVKSKIVELKDFT |
| | | | IHVDADSNSVIELPENFPPGSIAIFETGYVGIDDTLDHLIRS |
| | | | GALKATSNLNFESLNAVLFRSESEELDVSGNTFGAYNIPN |
| | | | HGKLVYCGLQGWVSVLRDIMINNDLAHPLSVNLREGHWA |
| | | | LDYILERLNLYNDKPGIRDVQAWLAERFDKIKQLPNFLVPS |
| | | | FFALTIGILYGCCRFKAMLLFPKYIGNANVFVQSLALTSIQM |
| | | | VSEVKSTSIFPNINVPCMCAGLPHFSTNYMRCWGRDVFIS |
| | | | FRGLLLVTGRYETAKQHILGFAKTTKHGLIPNLLDAGRNPR |
| | | | YNARDAAWFFLQAIQDYITIVPEGHSILSEKVSRRFPLNDK |
| | | | YITYEDPEAFSYSSSIEEIIYEILSRHAKGIKFREANAGPNLD |
| | | | RVMKDEGFNIEIAVDWSTGLIHGGNQFNCGTWMDKNGES |
| | | | EKAKSKGVPGTPRDGAAVEINGLLKSTLRFVLKLISEGKFK |
| | | | KTDVTRQDGSKISFADWNDLLQENFEKNYYIPLDPKDDNE |
| | | | YNTDSNIVNRRGIYKDLCGSGKPFEDYQLRPNQFIAMAVA |
| | | | PELFSPEHAMSAIKVADENIRGKLGMRTLDPSDYNYRPYY |
| | | | NNSEDSDDFATSKGRNYHQGPEWVWCYGYFMRSFHTF |
| | | | NFLANPLSRNETENRPSSYLMQQIYLRLDDHRKAIIESPW |
| | | | AGLPELTNKDGEFCQDSSPTQAWSSGCILDVLYDVWVAF |
| | | | DSKWYAKQIKQNNPHHHHHH |
| ARBA000 | Sporothrix | 336 | MIPKTMTSSEVYLLPLSDDGSPQVGGNQYIYLAATSEQPIV |
| 20723 | schenckii (strain | | VRFAIEGTSSICRHGSLWVNIPEKGAEFRRDHFREFKLDP |
| | ATCC 58251/de | | DFHRTIHISIPIYHPGAFAFYTTYAALPDLSTELAERASFDE |
| | Perez 2211183) | | EKKTTPRYYIDVAPRLRLDGRPLPLEALSVFSIISKFMGKY |
| | | | PNDWERHLRGISDRGYNMVHFTPLQQRGESNSPYSLYD |
| | | | QLAWDPICFPRGEQDVQDMVHSLKTNHSLLSLTDIVLNHT |
| | | | ANNSPWLLDHPDAGYNLKTAPHLESAYLLDSKLLELGDRL |
| | | | GEMGLPTDLKSTDDLDKIMGAIKTECIAGIRLWEYYVLDVE |
| | | | NDAANAADSWSRGDITIPDAYAGLNDLAFLKSADLKTQTE |
| | | | FLKKHALHGNDRLGERHRRTIDGKVGAALLTAVLGPASG |
| | | | GAEKDAAHNTLVSMADALNVPFYAEYDAEVADILQQLFNR |
| | | | IKYCRLDDNGPKMGPIGPKSALIETYFTRLPRNEQTAHFDI |
| | | | EELVLVNNGWVWGGNALVDNAGPQSRVYLRREVIVWGD |
| | | | CVKLRYGSGPADSPWLWEHMTQYARTMAKYFAGFRIDN |
| | | | CHSTPMHVAEHILDEARLVNPNLYVVAELFSGSEDTDYIFV |
| | | | KRLGLSSLIREAMQAWSTGEMSRLVHRHGGRPIGSFEVD |
| | | | DITRSDVAKKQQQSALPNANANGHTSEATADGEAEGDEL |
| | | | CREIIRRIRVVPVQALFMDCTHDNEAPAQKRDARDTLPNA |
| | | | ALVSMCASATGSVMGYDEIYPRLVDLVNETRMYTSESSS |
| | | | RAVKVGSGRNGIGGIKKLLNQIHVLMGKDGYAETHIHHEE |
| | | | EYVTVHRVHPESRKGYFLIAHTAYPGYGNGNGAFNPVHL |
| | | | TGTRANHLGSWMLEVDTSEEATKNALDDTKFLRGLPSRV |
| | | | VDLPGIKIDYNNGDTSIAVPDKFPPGSIALFETWIPAAEHSS |
| | | | GLDTFVTSGAKAAFGELSLVDLNFLLYRCEAEERDASGGN |
| | | | DGVYDLPGYGRLVYAGLQGWYSILKHVVRDNNLAHPLCQ |
| | | | NLRDGQWALDYIAGRLERVATKDTHSNLGKPAAWFKERF |
| | | | DAIRPIPAFLHPRYFGLIMKTAYQASWDRAMALMSANVRE |
| | | | GQWFVQSLAMVSIQQTGLVQSGSLYPNRLVPSLAAGLPH |
| | | | FAVEWARCWGRDVFISLRGLFLGTGRYAEAKEHILAFASV |
| | | | LKHGMVPNLLGSDGNPRYNARDATWFFLQCIQDYVNRAP |
| | | | DGDELLKTKLKRRFLPNDDTWFDANDPRAYSKESTVEEVI |
| | | | QEALQRHATGMSFREANAGPGLDMQMRDEGFNIDVHVD |
| | | | WSNGFIFGGNQFNCGTWMDKMGESERAGSKGIPGTPRD |
| | | | GAAIEITGLLYSTLKWLAHRHSKGGFAHQGVTRADGSSIS |
| | | | FADWAALIQDNFERCYYVPLQPADDAQYDVNPAVVNRRG |
| | | | IYKDLYKSGKEYEDYQLRPNFAIAMTVAADLFDAEHATHAL |
| | | | CLADTVLRGPTGMATLDPADLNYRPYYINSEDSTDFATSK |
| | | | GRNYHQGPEWLWPTGFFLRALLKFDLRRRAAQSPDDAE |
| | | | GRTEAFQQVTRRLMGCKEQIQESVWAGLTELTQKDGAY |
| | | | CQDSSPTQAWSAGCLIDLYMDAEEEQARHKTSHVAIR |
| ARBA000 | Sporothrix | 337 | MIPKTMTSSEVYLLPLSDDGSPQVGGNQYIYLAATSEQPIV |
| 20723 | schenckii (strain | | VRFAIEGTSSICRHGSLWVNIPEKGAEFRRDHFREFKLDP |
| (6X-His) | ATCC 58251/de | | DFHRTIHISIPIYHPGAFAFYTTYAALPDLSTELAERASFDE |
| | Perez 2211183) | | EKKTTPRYYIDVAPRLRLDGRPLPLEALSVFSIISKFMGKY |
| | | | PNDWERHLRGISDRGYNMVHFTPLQQRGESNSPYSLYD |
| | | | QLAWDPICFPRGEQDVQDMVHSLKTNHSLLSLTDIVLNHT |
| | | | ANNSPWLLDHPDAGYNLKTAPHLESAYLLDSKLLELGDRL |
| | | | GEMGLPTDLKSTDDLDKIMGAIKTECIAGIRLWEYYVLDVE |
| | | | NDAANAADSWSRGDITIPDAYAGLNDLAFLKSADLKTQTE |
| | | | FLKKHALHGNDRLGERHRRTIDGKVGAALLTAVLGPASG |
| | | | GAEKDAAHNTLVSMADALNVPFYAEYDAEVADILQQLFNR |
| | | | IKYCRLDDNGPKMGPIGPKSALIETYFTRLPRNEQTAHFDI |
| | | | EELVLVNNGWVWGGNALVDNAGPQSRVYLRREVIVWGD |
| | | | CVKLRYGSGPADSPWLWEHMTQYARTMAKYFAGFRIDN |
| | | | CHSTPMHVAEHILDEARLVNPNLYVVAELFSGSEDTDYIFV |
| | | | KRLGLSSLIREAMQAWSTGEMSRLVHRHGGRPIGSFEVD |
| | | | DITRSDVAKKQQQSALPNANANGHTSEATADGEAEGDEL |
| | | | CREIIRRIRVVPVQALFMDCTHDNEAPAQKRDARDTLPNA |
| | | | ALVSMCASATGSVMGYDEIYPRLVDLVNETRMYTSESSS |
| | | | RAVKVGSGRNGIGGIKKLLNQIHVLMGKDGYAETHIHHEE |
| | | | EYVTVHRVHPESRKGYFLIAHTAYPGYGNGNGAFNPVHL |
| | | | TGTRANHLGSWMLEVDTSEEATKNALDDTKFLRGLPSRV |
| | | | VDLPGIKIDYNNGDTSIAVPDKFPPGSIALFETWIPAAEHSS |
| | | | GLDTFVTSGAKAAFGELSLVDLNFLLYRCEAEERDASGGN |
| | | | DGVYDLPGYGRLVYAGLQGWYSILKHVVRDNNLAHPLCQ |
| | | | NLRDGQWALDYIAGRLERVATKDTHSNLGKPAAWFKERF |
| | | | DAIRPIPAFLHPRYFGLIMKTAYQASWDRAMALMSANVRE |
| | | | GQWFVQSLAMVSIQQTGLVQSGSLYPNRLVPSLAAGLPH |
| | | | FAVEWARCWGRDVFISLRGLFLGTGRYAEAKEHILAFASV |
| | | | LKHGMVPNLLGSDGNPRYNARDATWFFLQCIQDYVNRAP |
| | | | DGDELLKTKLKRRFLPNDDTWFDANDPRAYSKESTVEEVI |
| | | | QEALQRHATGMSFREANAGPGLDMQMRDEGFNIDVHVD |
| | | | WSNGFIFGGNQFNCGTWMDKMGESERAGSKGIPGTPRD |
| | | | GAAIEITGLLYSTLKWLAHRHSKGGFAHQGVTRADGSSIS |
| | | | FADWAALIQDNFERCYYVPLQPADDAQYDVNPAVVNRRG |
| | | | IYKDLYKSGKEYEDYQLRPNFAIAMTVAADLFDAEHATHAL |
| | | | CLADTVLRGPTGMATLDPADLNYRPYYINSEDSTDFATSK |
| | | | GRNYHQGPEWLWPTGFFLRALLKFDLRRRAAQSPDDAE |
| | | | GRTEAFQQVTRRLMGCKEQIQESVWAGLTELTQKDGAY |
| | | | CQDSSPTQAWSAGCLIDLYMDAEEEQARHKTSHVAIRHH |
| | | | HHHH |
| ARBA000 | Lichtheimia | 338 | MTIAAATPKNTILSTGTAGQSSSTLPLVPPLYALLLADDGS |
| 20723 | corymbifera | | PDKSNTSSKLIESPAFCKLLHGNILSRNEHLHIQKREKYVRI |
| | JMRC:FSU:9682 | | PLGNQPCILRFVLRKGSLAATSSTLPRLHTTYPIQGPFDRA |
| | | | TFHAVEFDADGICDIPLMTPGAFEYHVTFENNRKSERVGY |
| | | | FVVEPRLYQSSSHKQLPLDAIMIESMIPKWMGPLARWQP |
| | | | HIDLVHQTSYNMIHFAPMQQRGSSDSPYSIRDQLLFADDL |
| | | | FDNPSLLSREDRLKEVRQTIFNIQHEHGILCLSDVVWNHTS |
| | | | HDSVFLQDHPDAGYNLQNSPHLVPAYELDTALLELSKSID |
| | | | QHGLPQRLETRQDVDNVMSHIKNSIFPQLKLWEYDIIDAEI |
| | | | AIREFSSAITQLSSSHETHKDAYKGAEDVAGQPIKRQAELF |
| | | | GAYAVIEGTHGTRFHRSVDIPRAIAFVQSYAKEDEYKAVF |
| | | | QKLIEQYNLVWYELYDNDVKIALDNIKNRITYLRIDDNGPK |
| | | | WGEISASSPLVESYFTRVNETIQVANNGWIWDADPLEDFA |
| | | | GPESRAYLRRQVICWGDCVKLRYGKSFDDNPWLWDHMR |
| | | | QYTEQVASLFHGIRIDNCHSTPLHVAEYLLDCARRVRPDL |
| | | | YVIAELFTGSPEMDERFVSRLGIHALIREAMQAWDPHEMS |
| | | | RLVHRHGGKPVGSMDQFIRWKRISKDGNEYMLIPIEHGS |
| | | | MPRALFMDCTHDNKTPFQMRTAEDALANSALVAFADCAT |
| | | | GSVKGYDEIYPRLLDLVGEKRHYNPNVDASTTGIMRIKEQ |
| | | | LQHLHMKMALEGYREVHVHHEGDYVLVHRQHPRTHDGY |
| | | | FLVARTAFPDKHGSIDPIRLHRTRLSFMFGASLHVDTAAAA |
| | | | PLDTKHLVGLPSVLADLPEPPIHSRENGFVEIELPSDFTPG |
| | | | SILVFQTFAQIHDTSNVTQLVTSMPESVLESLDVLDCNVVL |
| | | | YRCQGEEFDATNGGGVYEVPGWGQMTYAGLEGFMAVL |
| | | | RPIIRDNDLGHPFCDNLRKGHWAMDYIQNRLEVYQKNFE |
| | | | HHHLKPLIQWFSSRFGVIRGLPDFLVPKYFSMAVHTAYEH |
| | | | VRDHALQCMAPFVHASLDPFIHQLALCSVQMLGVVPSTSL |
| | | | EPIKITPTLAAGLPHFTHGCMRTWGRDVFISLRGLLMVTG |
| | | | QWDAARQHILAFASTLRNGLLPNLLDAGRNPRYNARDAV |
| | | | WWFMQSVQDYCKLAPDGTDILSAKVSRRFPRDYEWVSH |
| | | | DHGYDWDCTLAELIQEIMQQHAKGIHFREHNAGPNIDQQ |
| | | | MKDEGFNIDIEVDWESGGVLIGGNIWNCGTWMDKMGES |
| | | | EKAGNKGYPGTPRDGAPIEITGLLKSALRWLSSLCNTDVF |
| | | | PWKGIDRANGKTITYSEWNDLLQSNFERIYFVPREPDQDT |
| | | | NYAVDPSIVHRRGIYKDAYGATEPYTEYQFRPNLCVAMVV |
| | | | APELFNRQHAKQCLQLMHNVLLAPLGMRTLDPADMRYRP |
| | | | YYINGEDSEDFDTAKGRNYHQGPEWIWPIGYYLRAAHQF |
| | | | GALSSLAISRVLHAHRQEIETSTWAGLPELTNKDGETCWD |
| | | | SCFSQAWSAATILDLLYDMQQ |
| ARBA000 | Lichtheimia | 339 | MTIAAATPKNTILSTGTAGQSSSTLPLVPPLYALLLADDGS |
| 20723 | corymbifera | | PDKSNTSSKLIESPAFCKLLHGNILSRNEHLHIQKREKYVRI |
| (6X-His) | JMRC:FSU:9682 | | PLGNQPCILRFVLRKGSLAATSSTLPRLHTTYPIQGPFDRA |
| | | | TFHAVEFDADGICDIPLMTPGAFEYHVTFENNRKSERVGY |
| | | | FVVEPRLYQSSSHKQLPLDAIMIESMIPKWMGPLARWQP |
| | | | HIDLVHQTSYNMIHFAPMQQRGSSDSPYSIRDQLLFADDL |
| | | | FDNPSLLSREDRLKEVRQTIFNIQHEHGILCLSDVVWNHTS |
| | | | HDSVFLQDHPDAGYNLQNSPHLVPAYELDTALLELSKSID |
| | | | QHGLPQRLETRQDVDNVMSHIKNSIFPQLKLWEYDIIDAEI |
| | | | AIREFSSAITQLSSSHETHKDAYKGAEDVAGQPIKRQAELF |
| | | | GAYAVIEGTHGTRFHRSVDIPRAIAFVQSYAKEDEYKAVF |
| | | | QKLIEQYNLVWYELYDNDVKIALDNIKNRITYLRIDDNGPK |
| | | | WGEISASSPLVESYFTRVNETIQVANNGWIWDADPLEDFA |
| | | | GPESRAYLRRQVICWGDCVKLRYGKSFDDNPWLWDHMR |
| | | | QYTEQVASLFHGIRIDNCHSTPLHVAEYLLDCARRVRPDL |
| | | | YVIAELFTGSPEMDERFVSRLGIHALIREAMQAWDPHEMS |
| | | | RLVHRHGGKPVGSMDQFIRWKRISKDGNEYMLIPIEHGS |
| | | | MPRALFMDCTHDNKTPFQMRTAEDALANSALVAFADCAT |
| | | | GSVKGYDEIYPRLLDLVGEKRHYNPNVDASTTGIMRIKEQ |
| | | | LQHLHMKMALEGYREVHVHHEGDYVLVHRQHPRTHDGY |
| | | | FLVARTAFPDKHGSIDPIRLHRTRLSFMFGASLHVDTAAAA |
| | | | PLDTKHLVGLPSVLADLPEPPIHSRENGFVEIELPSDFTPG |
| | | | SILVFQTFAQIHDTSNVTQLVTSMPESVLESLDVLDCNVVL |
| | | | YRCQGEEFDATNGGGVYEVPGWGQMTYAGLEGFMAVL |
| | | | RPIIRDNDLGHPFCDNLRKGHWAMDYIQNRLEVYQKNFE |
| | | | HHHLKPLIQWFSSRFGVIRGLPDFLVPKYFSMAVHTAYEH |
| | | | VRDHALQCMAPFVHASLDPFIHQLALCSVQMLGVVPSTSL |
| | | | EPIKITPTLAAGLPHFTHGCMRTWGRDVFISLRGLLMVTG |
| | | | QWDAARQHILAFASTLRNGLLPNLLDAGRNPRYNARDAV |
| | | | WWFMQSVQDYCKLAPDGTDILSAKVSRRFPRDYEWVSH |
| | | | DHGYDWDCTLAELIQEIMQQHAKGIHFREHNAGPNIDQQ |
| | | | MKDEGFNIDIEVDWESGGVLIGGNIWNCGTWMDKMGES |
| | | | EKAGNKGYPGTPRDGAPIEITGLLKSALRWLSSLCNTDVF |
| | | | PWKGIDRANGKTITYSEWNDLLQSNFERIYFVPREPDQDT |
| | | | NYAVDPSIVHRRGIYKDAYGATEPYTEYQFRPNLCVAMVV |
| | | | APELFNRQHAKQCLQLMHNVLLAPLGMRTLDPADMRYRP |
| | | | YYINGEDSEDFDTAKGRNYHQGPEWIWPIGYYLRAAHQF |
| | | | GALSSLAISRVLHAHRQEIETSTWAGLPELTNKDGETCWD |
| | | | SCFSQAWSAATILDLLYDMQQHHHHHH |
| ARBA000 | Protomyces | 340 | MTKIHLLELNDDGTPCVGTALVRLPAPFEPYTLRFVLYSTN |
| 20723 | lactucae-debilis | | QLDSGATLKINVPAAGQVFDRQKFLTKSLKAEAGTDACVD |
| | | | VEIDQPGAFAWRIEYLNEDAAAITTAEYYIVVDPALTINGTQ |
| | | | ISLDALAIQSVVPKWMGKLTQWSTYLEYMSSVKKYNMLHF |
| | | | CPLQQRGASDSPYSLFDQHKFSDDLFDNVSLSLKDRTAQ |
| | | | MEAMVNDMEHKYGLLSLTDVVWNHTASDSDWLQDHPEA |
| | | | GFNLHNSPHLCAAFALDTALLELSSNLSKLGLPTTLKSTQD |
| | | | LLAIMEAIKSRVLGTIKLWEFYVIDTQKALSTMVFTQQPAPV |
| | | | GELNSQSSLESIADFCLSQGVVQNHQFLGDRFSKIVDPAK |
| | | | LGAIINALCCAKTDKLAFASRILDEINLRFYQEHNADQDAF |
| | | | MEQLYNRIKYVRLDSHGPKMGPITSMAPLIETYFTRLPSN |
| | | | DRTRKHPSGSLALANNGWMWAANPLENFAGPQSKAYLR |
| | | | REVIVWGDCVKLRYGDCPADNPYLWEHMRLYTSQMASIF |
| | | | HGFRIDNCHSTPIPVARYLLDEARKSRPNLYVVAELFTGSE |
| | | | DMDTTFVQKLGICSLLREAMQAWSPDELSRLVHKYGPSPI |
| | | | GSIPNQLGNVDSSQLIKPAPIHALFFDCTHDNETPVQKRSA |
| | | | EDTLPNAALVAMTASAIGSVFGYDEVYPHILDLVSEKRHYS |
| | | | QRENGLGRAKGVLNAVHIEMGLKNYSEAHVHHEGAYITV |
| | | | HRVSPETKEGYFLIAHTAFHGGQELGAISPIRLSGTSLELV |
| | | | YAATLSVTPAQKDASEYTELDGVPASVVMLDFPVETTDDD |
| | | | DTILTVPGRFPPGSIALFRTKLRLPKLPESPELSWEKLEDF |
| | | | LMMGASEAVASCSLIDLNVLLYRCNAEEQDTVAEGAYNIP |
| | | | NYGQLVYAGLQGWMAPISAVTRSNDLGHPLCDHLRNGL |
| | | | WALDYIVAREQRYIRNGYDALSPSSRWLSTRFDYIKRLPN |
| | | | FLVPKAFVLVIKALYCAARERSLCLLSKQIRRGTKFTQDLA |
| | | | LCSLQMQGAVKSASIDPAKSIPCMAAGLPHFSTSWARCW |
| | | | GRDIFIAMRGLLLVTERYAEAESHILAFATTLKHGMIPNLLD |
| | | | SLRTPRYNSRDSIWFYMQAIQDYVTMVPNGAEILKKSVKR |
| | | | RFPLNDEYVEWDDQRAYSYQTSVTDVIQETLARHAQGLH |
| | | | FREANAGPKLDMQMSNEGFNIDVEVDWSTGFLHGGNQH |
| | | | NCGTWMDKMGESERAGSKGVPGTPRDGAAVEITGLLKS |
| | | | TLRWVNQLHQQGLYPYTCVRATIDGKETDMTFVRWEALI |
| | | | QSNFERCYYIPKQSSDDAQYEVDAAIVNRRGIYKDLYKSG |
| | | | KAYEDYQLRAQVPMAMTVAPELFETEHALSHLETADHFL |
| | | | RGPLGMATLDPIDNNYRPYYNNTEDSTDFHTAKGRNYHQ |
| | | | GPEWVFPVGYFLRAMLHFQIQDGRTNSDEISRQIYRRLSA |
| | | | HRTEIMSTPWTGLTELTNKAGALCGDSSPTQAWSSGTLL |
| | | | DVIHDMQELRVKQL |
| ARBA000 | Protomyces | 341 | MTKIHLLELNDDGTPCVGTALVRLPAPFEPYTLRFVLYSTN |
| 20723 | lactucae-debilis | | QLDSGATLKINVPAAGQVFDRQKFLTKSLKAEAGTDACVD |
| (6X-His) | | | VEIDQPGAFAWRIEYLNEDAAAITTAEYYIVVDPALTINGTQ |
| | | | ISLDALAIQSVVPKWMGKLTQWSTYLEYMSSVKKYNMLHF |
| | | | CPLQQRGASDSPYSLFDQHKFSDDLFDNVSLSLKDRTAQ |
| | | | MEAMVNDMEHKYGLLSLTDVVWNHTASDSDWLQDHPEA |
| | | | GFNLHNSPHLCAAFALDTALLELSSNLSKLGLPTTLKSTQD |
| | | | LLAIMEAIKSRVLGTIKLWEFYVIDTQKALSTMVFTQQPAPV |
| | | | GELNSQSSLESIADFCLSQGVVQNHQFLGDRFSKIVDPAK |
| | | | LGAIINALCCAKTDKLAFASRILDEINLRFYQEHNADQDAF |
| | | | MEQLYNRIKYVRLDSHGPKMGPITSMAPLIETYFTRLPSN |
| | | | DRTRKHPSGSLALANNGWMWAANPLENFAGPQSKAYLR |
| | | | REVIVWGDCVKLRYGDCPADNPYLWEHMRLYTSQMASIF |
| | | | HGFRIDNCHSTPIPVARYLLDEARKSRPNLYVVAELFTGSE |
| | | | DMDTTFVQKLGICSLLREAMQAWSPDELSRLVHKYGPSPI |
| | | | GSIPNQLGNVDSSQLIKPAPIHALFFDCTHDNETPVQKRSA |
| | | | EDTLPNAALVAMTASAIGSVFGYDEVYPHILDLVSEKRHYS |
| | | | QRENGLGRAKGVLNAVHIEMGLKNYSEAHVHHEGAYITV |
| | | | HRVSPETKEGYFLIAHTAFHGGQELGAISPIRLSGTSLELV |
| | | | YAATLSVTPAQKDASEYTELDGVPASVVMLDFPVETTDDD |
| | | | DTILTVPGRFPPGSIALFRTKLRLPKLPESPELSWEKLEDF |
| | | | LMMGASEAVASCSLIDLNVLLYRCNAEEQDTVAEGAYNIP |
| | | | NYGQLVYAGLQGWMAPISAVTRSNDLGHPLCDHLRNGL |
| | | | WALDYIVAREQRYIRNGYDALSPSSRWLSTRFDYIKRLPN |
| | | | FLVPKAFVLVIKALYCAARERSLCLLSKQIRRGTKFTQDLA |
| | | | LCSLQMQGAVKSASIDPAKSIPCMAAGLPHFSTSWARCW |
| | | | GRDIFIAMRGLLLVTERYAEAESHILAFATTLKHGMIPNLLD |
| | | | SLRTPRYNSRDSIWFYMQAIQDYVTMVPNGAEILKKSVKR |
| | | | RFPLNDEYVEWDDQRAYSYQTSVTDVIQETLARHAQGLH |
| | | | FREANAGPKLDMQMSNEGFNIDVEVDWSTGFLHGGNQH |
| | | | NCGTWMDKMGESERAGSKGVPGTPRDGAAVEITGLLKS |
| | | | TLRWVNQLHQQGLYPYTCVRATIDGKETDMTFVRWEALI |
| | | | QSNFERCYYIPKQSSDDAQYEVDAAIVNRRGIYKDLYKSG |
| | | | KAYEDYQLRAQVPMAMTVAPELFETEHALSHLETADHFL |
| | | | RGPLGMATLDPIDNNYRPYYNNTEDSTDFHTAKGRNYHQ |
| | | | GPEWVFPVGYFLRAMLHFQIQDGRTNSDEISRQIYRRLSA |
| | | | HRTEIMSTPWTGLTELTNKAGALCGDSSPTQAWSSGTLL |
| | | | DVIHDMQELRVKQLHHHHHH |
| ARBA000 | Tuber magnatum | 342 | MSPPLVYMLPLTDTGAPDIAGDHIGLPPPREPYTLRIAIEG |
| 20723 | | | ASSITRNGSLWTNVPAKGDNFGRNKFREFKLTPSFSRTLH |
| | | | IDVPIALPGTFCYYITYDPLPSLKELSSPTNPSGGAKTGTH |
| | | | WFTVAARLSVNGEPLALDSLSCFSVVSKWLGPVEKWDGY |
| | | | MRYIAEKGYNMVHFTPMMHRGESNSPYSIYDQLAFDPEA |
| | | | FPNGTDDVALIVDRMEKRHGLLSLTDVVWNHTAHNSLWL |
| | | | QSHPESGYNLVTAPWLEAAFELDTALLDFGNHLEALGFPT |
| | | | ELETQEDLLKIMDGVKTHVIGALRLWEFYVVNVEAATDAVI |
| | | | EAYISGKSDSKLIDTAEYKSGLSLHDMTQFLIKNGLKGADIL |
| | | | GSRYSRTIDPGVGAAFLALECGKPDPKNAKGVEEAKQALI |
| | | | RVYNRITYLRLDSNGPKMGKITKESPIIETYFTRLPKNEITA |
| | | | QHDPKSLALANNGWIWSNVADFAGPKSRSYLRREVIVWG |
| | | | DCVKLRFGDKPEDNPFLWQFMVEYTKLMARCFHGFRIDN |
| | | | CHSTPIHVGQYLLDIAREVREDLYVVAELFTGSEHQDRRF |
| | | | VEHLGLGSLVREAMVAWGPGELSRLVHRHGGKPIGSLRQ |
| | | | ELWVRAVTASPIHALFMDCTHDNETPTQKRTPQDTLPNGA |
| | | | LVAMCDCGIGSVMGYDEIYPKLLNLVTEPRIYYPPPEKIDE |
| | | | TTPGIGKVKGLLNRIHTEMGKDGFTEMHVHHEGEYITVHR |
| | | | VHPKSHKGYFLIAHTAFGPGDHRGDFNPITVTGTKVKVIG |
| | | | SWTLVADGSDERKEKVLSQPDYLTGLPAKLVEVESPKIGE |
| | | | NGNTTTITVPEKFPPGSIALLETWIPGTDGESLAQFVTSGA |
| | | | EEAFANTSLVDLNFVLYRCESEERDSSDDRDGCYNIPGY |
| | | | GALVYAGLQGWWSVLKDVIRNNDLAHPICQHLRDGQWAL |
| | | | DYIVGRLEKLEARGNKGLAEPTEWLKERFDRIRSVPGFLL |
| | | | PRYFALVMQTAYTTAVERATSFFTEDIREGTAFLKELSLVS |
| | | | LQMTGLMDSTSLWPGKKVACMAAGLPHFSYDYMRCWG |
| | | | RDVFISIRGLYLATGRYEDAKEHILAFGSVLKHGMIPNLLD |
| | | | GGRKPRYNARDSIWFYLQAVQDYTGIVPNGIELLREKVKR |
| | | | RFLPFDDTWFNYDDPRAYSRESTIAEIIHEALERHALGFEF |
| | | | LEANAGPGLDNQMKWEGFTVGVKPDWTTGIIKGGSQFN |
| | | | CGTWMDKMGESERAGNKGVPGTPRDGAAIEITGLLYSTL |
| | | | KWVAELRKKKLYKWDSVKTSQGKGITFDMWADLLKKNFE |
| | | | KLYYVPEDPGEDSKYSIKPELIGRRGIYKDVWGGGKEYED |
| | | | YQFRPNFAIAMTVAPDLFVPKHAIRCLEIADEVLRGPTGMA |
| | | | TLDPIDKDYRPYYRNSEDSDDFATSKGRNYHQGPEWLW |
| | | | PTGFYLRALLTFSPPNIETLQQINLRMDGCRKAINESPWA |
| | | | GLTELTNKNGEICYDSCPTQAWSASCLIDLCYDAAKIKL |
| ARBA000 | Tuber magnatum | 343 | MSPPLVYMLPLTDTGAPDIAGDHIGLPPPREPYTLRIAIEG |
| 20723 | | | ASSITRNGSLWTNVPAKGDNFGRNKFREFKLTPSFSRTLH |
| (6X-His) | | | IDVPIALPGTFCYYITYDPLPSLKELSSPTNPSGGAKTGTH |
| | | | WFTVAARLSVNGEPLALDSLSCFSVVSKWLGPVEKWDGY |
| | | | MRYIAEKGYNMVHFTPMMHRGESNSPYSIYDQLAFDPEA |
| | | | FPNGTDDVALIVDRMEKRHGLLSLTDVVWNHTAHNSLWL |
| | | | QSHPESGYNLVTAPWLEAAFELDTALLDFGNHLEALGFPT |
| | | | ELETQEDLLKIMDGVKTHVIGALRLWEFYVVNVEAATDAVI |
| | | | EAYISGKSDSKLIDTAEYKSGLSLHDMTQFLIKNGLKGADIL |
| | | | GSRYSRTIDPGVGAAFLALECGKPDPKNAKGVEEAKQALI |
| | | | RVYNRITYLRLDSNGPKMGKITKESPIIETYFTRLPKNEITA |
| | | | QHDPKSLALANNGWIWSNVADFAGPKSRSYLRREVIVWG |
| | | | DCVKLRFGDKPEDNPFLWQFMVEYTKLMARCFHGFRIDN |
| | | | CHSTPIHVGQYLLDIAREVREDLYVVAELFTGSEHQDRRF |
| | | | VEHLGLGSLVREAMVAWGPGELSRLVHRHGGKPIGSLRQ |
| | | | ELVVRAVTASPIHALFMDCTHDNETPTQKRTPQDTLPNGA |
| | | | LVAMCDCGIGSVMGYDEIYPKLLNLVTEPRIYYPPPEKIDE |
| | | | TTPGIGKVKGLLNRIHTEMGKDGFTEMHVHHEGEYITVHR |
| | | | VHPKSHKGYFLIAHTAFGPGDHRGDFNPITVTGTKVKVIG |
| | | | SWTLVADGSDERKEKVLSQPDYLTGLPAKLVEVESPKIGE |
| | | | NGNTTTITVPEKFPPGSIALLETWIPGTDGESLAQFVTSGA |
| | | | EEAFANTSLVDLNFVLYRCESEERDSSDDRDGCYNIPGY |
| | | | GALVYAGLQGWWSVLKDVIRNNDLAHPICQHLRDGQWAL |
| | | | DYIVGRLEKLEARGNKGLAEPTEWLKERFDRIRSVPGFLL |
| | | | PRYFALVMQTAYTTAVERATSFFTEDIREGTAFLKELSLVS |
| | | | LQMTGLMDSTSLWPGKKVACMAAGLPHFSYDYMRCWG |
| | | | RDVFISIRGLYLATGRYEDAKEHILAFGSVLKHGMIPNLLD |
| | | | GGRKPRYNARDSIWFYLQAVQDYTGIVPNGIELLREKVKR |
| | | | RFLPFDDTWFNYDDPRAYSRESTIAEIIHEALERHALGFEF |
| | | | LEANAGPGLDNQMKWEGFTVGVKPDWTTGIIKGGSQFN |
| | | | CGTWMDKMGESERAGNKGVPGTPRDGAAIEITGLLYSTL |
| | | | KWVAELRKKKLYKWDSVKTSQGKGITFDMWADLLKKNFE |
| | | | KLYYVPEDPGEDSKYSIKPELIGRRGIYKDVWGGGKEYED |
| | | | YQFRPNFAIAMTVAPDLFVPKHAIRCLEIADEVLRGPTGMA |
| | | | TLDPIDKDYRPYYRNSEDSDDFATSKGRNYHQGPEWLW |
| | | | PTGFYLRALLTFSPPNIETLQQINLRMDGCRKAINESPWA |
| | | | GLTELTNKNGEICYDSCPTQAWSASCLIDLCYDAAKIKLHH |
| | | | HHHH |
| VEU20737.1 | Brettanomyces | 344 | MTRVVLLRLDDKGEPLVNGHGVLSFPAVPPEPTRKSDDP |
| | naardenensis | | LFCLRLHIQAGSRIANNGKVWTDVPPSGSIAFSREKFYGQ |
| | | | SILGSFDKDSTVDINVYWPGAYCYYLSYNSLDDDDNETLK |
| | | | TTLKFHFVVPPSLFISQKYLPLNSISMQSVISKWIGTGRDD |
| | | | FDRLFSEIQRKGYNMIHFTPLQARGESDSPYSICDQLEFD |
| | | | PALFPGGVDDVSRMIAELETRHGILSMTDVVFNHTANNSP |
| | | | WLREHPEAGYNLETAPHLEAAMELDALLLHFSRYMSWHG |
| | | | CPTNIRTTADLLKVMDGIKIHVLGQLRLWQYYVIDVKGELA |
| | | | RLKGEWDASRNGKGGKVDPAIFSDFPPDARSDLRVAARY |
| | | | IAATCAVKPFGLGARYENALDTVKLAAVLEKIFDKKLVDLD |
| | | | FPDLEKRTHAILDEVNLPLYKEYDEDNEDILENLYNRINYQ |
| | | | RLDSHGPQLGEVTKDSPLTEPYFTRFTDVNGRKWALANN |
| | | | GWIWDGNPLVDFASSSSKCYMRREVIVWGDCVKLRYGS |
| | | | GPQDSPYLWDRMTKYAQLCAKVFHGFRIDNCHSTPIHVG |
| | | | EALLDAARAIRPNLYVVAELFTGNEDLDTHFVERLGISSLIR |
| | | | EAMQAYSVGELSRLVHRHGGRPIGSFRWLPLDVLAYPAD |
| | | | KLEFAERQAEEIRRRSEIPVPELVTAAPTHALFMDCTHDN |
| | | | EMPNDKRTVEDTLPNAALVAFCACATGTTMGYDECYPHL |
| | | | LDLVKGKQMYTYGPGIGIGDVKAKLNSLRRVLAEQSLSDP |
| | | | EANEMHVHHEGQYITIHRSNAQTGEGYFLIARTKFCPDGE |
| | | | QTLSPIVLHGTLVKNEFAYALERLSAFTRVRSGETDTEEVD |
| | | | PLSKAAAPSDFITPVPVKLRTIEPADCLYDPVEHSTTVILPK |
| | | | KFPQGAILVLSTRIPNCDAELDKYVRTGAIEAAEELTLVDLN |
| | | | ALLYRCESEERDASGGVDGVYDIPDFGRLVYAGLQGWVS |
| | | | VLRGMVASNDLAHPLAKHLRNGHWALDYIPARIAKYEAAA |
| | | | SYAGDDDRAAAIERFRTWIESRFARVKEVPYFLVPRFFAL |
| | | | WVGVAYEALRFRALALMSPPVKRSTVFVQSLALVSVQMA |
| | | | GLTRTAPLSPFSTNEPSLAAGLPHFSFDFMRCWGRDVFIS |
| | | | LRGLLLATGRFDVAKSHILNFAMTLKHGLIPNLLGAGKEPR |
| | | | YNARDAVWWFLQAIQDLYHSAPIGEGDELLNARVRRRFP |
| | | | LDDTWVPWDDARAFAVESTLVQIIYEILSRHAAGIKFREAH |
| | | | AGPQIDSQMRDEGFNVEIHVDWTTGLVSGGNQFNCGTW |
| | | | MDKMGESVRAGNKGIPGTPRDGADVEINGLLKSALRFVIE |
| | | | LNEKGLFPYTSITTQDGKDVTFVQWNQLLQANFEKCFYIP |
| | | | ENPSEDKDFVVDSNIVNRRGIYKDIFGSGKPYEDYQLRGN |
| | | | FPIAIVVAPELFKPERALRAIDLADRVLRGPVGLKTLDPSDL |
| | | | NYRPYYNNSEDSNDFATSKGRNYHQGPEWVWIYGYFLR |
| | | | AYRMLHEKYDDRCKNESYLDQLMARRLSGNLKWLKESQ |
| | | | WAGLAELTNKDGEFCSDSCMTQAWSASCLVDVYMDYW |
| | | | RGESKEHLL |
| VEU20737.1 | Brettanomyces | 345 | MTRVVLLRLDDKGEPLVNGHGVLSFPAVPPEPTRKSDDP |
| (6X-His) | naardenensis | | LFCLRLHIQAGSRIANNGKVWTDVPPSGSIAFSREKFYGQ |
| | | | SILGSFDKDSTVDINVYWPGAYCYYLSYNSLDDDDNETLK |
| | | | TTLKFHFVVPPSLFISQKYLPLNSISMQSVISKWIGTGRDD |
| | | | FDRLFSEIQRKGYNMIHFTPLQARGESDSPYSICDQLEFD |
| | | | PALFPGGVDDVSRMIAELETRHGILSMTDVVFNHTANNSP |
| | | | WLREHPEAGYNLETAPHLEAAMELDALLLHFSRYMSWHG |
| | | | CPTNIRTTADLLKVMDGIKIHVLGQLRLWQYYVIDVKGELA |
| | | | RLKGEWDASRNGKGGKVDPAIFSDFPPDARSDLRVAARY |
| | | | IAATCAVKPFGLGARYENALDTVKLAAVLEKIFDKKLVDLD |
| | | | FPDLEKRTHAILDEVNLPLYKEYDEDNEDILENLYNRINYQ |
| | | | RLDSHGPQLGEVTKDSPLTEPYFTRFTDVNGRKWALANN |
| | | | GWIWDGNPLVDFASSSSKCYMRREVIVWGDCVKLRYGS |
| | | | GPQDSPYLWDRMTKYAQLCAKVFHGFRIDNCHSTPIHVG |
| | | | EALLDAARAIRPNLYVVAELFTGNEDLDTHFVERLGISSLIR |
| | | | EAMQAYSVGELSRLVHRHGGRPIGSFRWLPLDVLAYPAD |
| | | | KLEFAERQAEEIRRRSEIPVPELVTAAPTHALFMDCTHDN |
| | | | EMPNDKRTVEDTLPNAALVAFCACATGTTMGYDECYPHL |
| | | | LDLVKGKQMYTYGPGIGIGDVKAKLNSLRRVLAEQSLSDP |
| | | | EANEMHVHHEGQYITIHRSNAQTGEGYFLIARTKFCPDGE |
| | | | QTLSPIVLHGTLVKNEFAYALERLSAFTRVRSGETDTEEVD |
| | | | PLSKAAAPSDFITPVPVKLRTIEPADCLYDPVEHSTTVILPK |
| | | | KFPQGAILVLSTRIPNCDAELDKYVRTGAIEAAEELTLVDLN |
| | | | ALLYRCESEERDASGGVDGVYDIPDFGRLVYAGLQGWVS |
| | | | VLRGMVASNDLAHPLAKHLRNGHWALDYIPARIAKYEAAA |
| | | | SYAGDDDRAAAIERFRTWIESRFARVKEVPYFLVPRFFAL |
| | | | WVGVAYEALRFRALALMSPPVKRSTVFVQSLALVSVQMA |
| | | | GLTRTAPLSPFSTNEPSLAAGLPHFSFDFMRCWGRDVFIS |
| | | | LRGLLLATGRFDVAKSHILNFAMTLKHGLIPNLLGAGKEPR |
| | | | YNARDAVWWFLQAIQDLYHSAPIGEGDELLNARVRRRFP |
| | | | LDDTWVPWDDARAFAVESTLVQIIYEILSRHAAGIKFREAH |
| | | | AGPQIDSQMRDEGFNVEIHVDWTTGLVSGGNQFNCGTW |
| | | | MDKMGESVRAGNKGIPGTPRDGADVEINGLLKSALRFVIE |
| | | | LNEKGLFPYTSITTQDGKDVTFVQWNQLLQANFEKCFYIP |
| | | | ENPSEDKDFVVDSNIVNRRGIYKDIFGSGKPYEDYQLRGN |
| | | | FPIAIVVAPELFKPERALRAIDLADRVLRGPVGLKTLDPSDL |
| | | | NYRPYYNNSEDSNDFATSKGRNYHQGPEWVWIYGYFLR |
| | | | AYRMLHEKYDDRCKNESYLDQLMARRLSGNLKWLKESQ |
| | | | WAGLAELTNKDGEFCSDSCMTQAWSASCLVDVYMDYW |
| | | | RGESKEHLLHHHHHH |
| KAA8910486. | Trichomonascus | 346 | MASQVFVLPLSDMGEPQPGPGGDFINLPIPYESYKLRFVIF |
| 1 | ciferrii | | STSSVTNESSLWCNVPQDPSAPFERNKFYEYPIRSQFNK |
| | | | DTYVDVDIHTPGTYSFYITYLPISQNYFERYEEKHANGEND |
| | | | EMMASQNPVERSLFKETYIAMDVKGHLASTRKYYFSVSA |
| | | | GFTLNNNPLPLNALSIQTVISKWMGEYSTWDKKLAQIKGK |
| | | | GYNMIHFTPLQERGDSDSPYSIFDQLKWDPKCFKNGEDD |
| | | | VKDLVETMEKKHCLLSMTDIVLNHTANNSEWLKHHPDAG |
| | | | YSVYTAPHLRPALELDDELTAYSGRLKELRLPTVLETEDD |
| | | | VDNVILGINRNVLQKIRLWEFYVVDVKSTIKRVREVVENDE |
| | | | RFNAIYPTRVPPNLEDDLTDLAKYVVAEAGSGFYEFGPRFI |
| | | | KAIDADYFVSILKALFPDPNVEPSIIVEKATDILNEINSPLYK |
| | | | DYDFDKAEIISNLRGRLKYLRLEDGGPKLGEITAEKPIHESY |
| | | | FTKVKTEPKGEVVSLVNNGFIWNGDPMIDFASSNSKAYLR |
| | | | REVISWGDCVKLRYGSKPADSPFLWEHMTKYTQTMAKYF |
| | | | HGFRLDNCHSTPIHVAEYLLDKARLIRSNLYVAAELFTGSE |
| | | | SKDRVFVRRLGITSLIREAMQAWSPGELSRLVHKHGGRPI |
| | | | GSFSKQPLVKHAQLQENPQNVHFIRTTPIHALFMDCTHDN |
| | | | EMPFQKRTVEDTISTAALVSMCACAVGSTMGFDECYPHH |
| | | | LNVVHEKRPYTFGSGISEVKATLLDIHEDMGCKNAEEMFV |
| | | | HHEGQYITVHRVNPKEGTGWFLIARTKFSEDDDQKSKSPL |
| | | | PNGHPYAINDITLPGTQAVVHKTAALRVTGEYEDDENQLN |
| | | | GIPTEIVHVEEPEITYNENTENTTIKFKDAIPQGSFVLLRTFV |
| | | | TAIEYGLDKFVRSGADEALEKVDLLDLNVIMYRCEAEERD |
| | | | ASDGKDGVYWVPNYGPLTYAGLHGWIHPVREMVALNDTA |
| | | | HPIADNLREGNWAMDFAISRLFKYLKHYPNLKSFIDWLES |
| | | | RFNRVRAIPQVLVPRYFALVIFTAYTACFRRAMNLMPGSL |
| | | | KRSTMFLNNLALVSVQMVGKVPSASVFPFETVGSMAAGL |
| | | | PHFSSGYMRCWGRDVFLSFKGLLLSTGRFEEAKNHILGF |
| | | | AATLKHGLVPNLLDSGRYPRYNARDATWFFLQSIQDYCK |
| | | | AVPHGIKILDEKVKRRFPLDDTFVELGDPRAFSEESTIREVI |
| | | | YEIFARHAKGIQFREANAGPDIDQQMKDEGFNQNIYVDWK |
| | | | NGIIFGGNQWNCGTWMDKMGESEKAGNKGYPGTPRDG |
| | | | AAIEITGLLKSALRWVNELRVEEEFPWNTVTNQDGDEVSFI |
| | | | EWERKLQRSFEKAYYIPKDPKKDILFDVDSKIVHRRGIYKD |
| | | | LYRSGKPYEDYQLRPNFAIAMTVAPELFDVDRAMGALAAA |
| | | | DTIILGPVGMRTLDPDDYNYRPYYENSIDNDDFATSKGRN |
| | | | YHQGPEWVWLRGYYLRAMLQFDVLRKKRYGGDMKETF |
| | | | QQLHLRMIELSLWIRQSAWGGLTELTNKDGAICSDSSPTQ |
| | | | AWSAATIIDLFEDSKHYLEYSKAHHKKPSY |
| KAA8910486. | Trichomonascus | 347 | MASQVFVLPLSDMGEPQPGPGGDFINLPIPYESYKLRFVIF |
| 1 | ciferrii | | STSSVTNESSLWCNVPQDPSAPFERNKFYEYPIRSQFNK |
| (6X-His) | | | DTYVDVDIHTPGTYSFYITYLPISQNYFERYEEKHANGFND |
| | | | EMMASQNPVERSLFKETYIAMDVKGHLASTRKYYFSVSA |
| | | | GFTLNNNPLPLNALSIQTVISKWMGEYSTWDKKLAQIKGK |
| | | | GYNMIHFTPLQERGDSDSPYSIFDQLKWDPKCFKNGEDD |
| | | | VKDLVETMEKKHCLLSMTDIVLNHTANNSEWLKHHPDAG |
| | | | YSVYTAPHLRPALELDDELTAYSGRLKELRLPTVLETEDD |
| | | | VDNVILGINRNVLQKIRLWEFYVVDVKSTIKRVREVVENDE |
| | | | RFNAIYPTRVPPNLEDDLTDLAKYVVAEAGSGFYEFGPRFI |
| | | | KAIDADYFVSILKALFPDPNVEPSIIVEKATDILNEINSPLYK |
| | | | DYDFDKAEIISNLRGRLKYLRLEDGGPKLGEITAEKPIHESY |
| | | | FTKVKTEPKGEVVSLVNNGFIWNGDPMIDFASSNSKAYLR |
| | | | REVISWGDCVKLRYGSKPADSPFLWEHMTKYTQTMAKYF |
| | | | HGFRLDNCHSTPIHVAEYLLDKARLIRSNLYVAAELFTGSE |
| | | | SKDRVFVRRLGITSLIREAMQAWSPGELSRLVHKHGGRPI |
| | | | GSFSKQPLVKHAQLQENPQNVHFIRTTPIHALFMDCTHDN |
| | | | EMPFQKRTVEDTISTAALVSMCACAVGSTMGFDECYPHH |
| | | | LNVVHEKRPYTFGSGISEVKATLLDIHEDMGCKNAEEMFV |
| | | | HHEGQYITVHRVNPKEGTGWFLIARTKFSEDDDQKSKSPL |
| | | | PNGHPYAINDITLPGTQAVVHKTAALRVTGEYEDDENQLN |
| | | | GIPTEIVHVEEPEITYNENTENTTIKFKDAIPQGSFVLLRTFV |
| | | | TAIEYGLDKFVRSGADEALEKVDLLDLNVIMYRCEAEERD |
| | | | ASDGKDGVYVVPNYGPLTYAGLHGWIHPVREMVALNDTA |
| | | | HPIADNLREGNWAMDFAISRLFKYLKHYPNLKSFIDWLES |
| | | | RFNRVRAIPQVLVPRYFALVIFTAYTACFRRAMNLMPGSL |
| | | | KRSTMFLNNLALVSVQMVGKVPSASVFPFETVGSMAAGL |
| | | | PHFSSGYMRCWGRDVFLSFKGLLLSTGRFEEAKNHILGF |
| | | | AATLKHGLVPNLLDSGRYPRYNARDATWFFLQSIQDYCK |
| | | | AVPHGIKILDEKVKRRFPLDDTFVELGDPRAFSEESTIREVI |
| | | | YEIFARHAKGIQFREANAGPDIDQQMKDEGFNQNIYVDWK |
| | | | NGIIFGGNQWNCGTWMDKMGESEKAGNKGYPGTPRDG |
| | | | AAIEITGLLKSALRWVNELRVEEEFPWNTVTNQDGDEVSFI |
| | | | EWERKLQRSFEKAYYIPKDPKKDILFDVDSKIVHRRGIYKD |
| | | | LYRSGKPYEDYQLRPNFAIAMTVAPELFDVDRAMGALAAA |
| | | | DTIILGPVGMRTLDPDDYNYRPYYENSIDNDDFATSKGRN |
| | | | YHQGPEWVWLRGYYLRAMLQFDVLRKKRYGGDMKETF |
| | | | QQLHLRMIELSLWIRQSAWGGLTELTNKDGAICSDSSPTQ |
| | | | AWSAATIIDLFEDSKHYLEYSKAHHKKPSYHHHHHH |
| ARBA000 | Mortierella | 348 | MPYTPVDIRRNRLKASAAPKSFKAKPQNDDVLRIYTLELE |
| 20723 | isabellina | | DDGSPEYSKRYIRLAPTQKDVFVIRFKVTAGMLASNNGVL |
| | | | YTNYPISGCFDREKFHEVRFSSDFSKDTYCDVTVTTPGAY |
| | | | EYYVEYEPINQENSDNQPEPKRSSRSGYFVVEPRLYIDSP |
| | | | VKPSEPSAEKKEVQEHAPAKVLLPLDGLVIESAVGKWLGP |
| | | | LDTWDAHLQHMKAAGYNMIHFVPLQVRGASNSPYSIHDQ |
| | | | TAFSDDLFNEQDVKKSSEEKAQLVNKQLHRIQDEYGIMCL |
| | | | SDVVWNHTSFDSVWLQDHPESGYNLDNSPHLQAAFELD |
| | | | TALINLVDDFSKYSLPTTLKSGQDLDAVMNVIKNNVFPDLK |
| | | | LWEYKVIDVVSEKKKLHEALLAAPKETAQSQVFENANLSG |
| | | | LASDQQAQLLAEHGIADRQSGSRYGKTVDIDVAVAFIKAL |
| | | | GGVASGEQVSEDVAAKLVDIYGKVLDVYNLPFYKEYDED |
| | | | VRIALENTKNRIEYTRLADHGPKWGEITKEKPIVDNYFTRL |
| | | | PKNDKTKGHSEGSLALANNGWIWNADPMQDFAGPQSAA |
| | | | YLRREVICWGDCVKLRYGSGADDNPYLWKHMKDYTEQT |
| | | | AKYFQGIRIDNCHSTPIHVAQYLLDAARKIRPDLYVVAELFT |
| | | | GSEDSDVTFVSKLGINSLIREAMQAWDAAELSRLVHRHG |
| | | | GKAIGSMDQALTTEQINYSLNGKEEEPALLVPVLQGSVPH |
| | | | ALFMDCTHDNETPNEKRTPQDTLPNAALVAFSGSAIGSVK |
| | | | GYDDIYPRLLDIVNETRLYDLPKPDANVGINKVKPVLQKLH |
| | | | TEMIMDGYIECHVHHEGDYIIVHRQHPELHTGYLLVAHTAF |
| | | | PGRQNNRGSISPIKLRGTDVDIMFSATLDIESRENLSDEKT |
| | | | LRGLKSKIVSLSAPTIKSLSDNKGRYTEITVPASFPSGSIMV |
| | | | LKTWIQDNPAESYDLISSCSDDVFKNMTSLDMNWVLHRCE |
| | | | GEERDATGEGVYNIPGFGGLPYAGLEGFVSVLKPIIAHND |
| | | | LGHPFCAHLREGTWAMDFIVDRLNRYIGNYPNLKRLKDW |
| | | | FASRFSEVKGLPDFLLPKYFALIIRTAYLKARAHAVSLMSPL |
| | | | VREGDRFIQSLALCSVQMYGIVPSSGLHPTEVTPSMAAGL |
| | | | PHFTHSYMRTWGRDVFIALRGLFLTTGNYDAAKRHIVSFA |
| | | | SSLKHGLIPNLLDAVRHPRYNSRDSVWWFLQAVQDYYNM |
| | | | APDGKSILQEKVSRRFPKNDTFVPPEEGFKYSCTVAEIIQE |
| | | | IFERHAAGIHFREHNAGEQLDRQMLDEGFNIDIDVDWESG |
| | | | TLIGGNRFNCGTWQDKMGESVKAGNKGLPATPRDGAPIE |
| | | | ITGLLKSTLRWVVELHERGEFQWTSAKDKSGNERSYKDW |
| | | | NDLLQTNFERVYYVPLDEKEDSQYDVLSQIVNRRGIYKDV |
| | | | YKSKEPYTDYQFRSNVPVAMVVAPELFTPAKAMGSLELA |
| | | | RSNLLGPLGMATLDPSDNEYHPDYHNSDDSDNKAVAKG |
| | | | WNYHQGPEWVWQTGYFLRAYLYFSLKTGEDQSKREKVM |
| | | | DIQRILLAHKREIESSPWAGLPELTNRNGNPCWDSCPTQA |
| | | | CYDDEVVLIQSQNTSFNHRSPATSDQESAAINFFLFCANLI |
| | | | SEEPMNNDQVIPLETHKLGLELRPHPQRGRGVFATCALK |
| | | | ARTLVEISPILLVNHDEYSAHGQYTILDHYTYRWEGGYALA |
| | | | LGLGSMFNHAKNPNVGFVRDIPNAVIRYFTLRAIEPAEELC |
| | | | ISYGDHLWFEDTDAKSEASIDSSEDEWFGKIMVDDEE |
| ARBA000 | Mortierella | 349 | MPYTPVDIRRNRLKASAAPKSFKAKPQNDDVLRIYTLELE |
| 20723 | isabellina | | DDGSPEYSKRYIRLAPTQKDVFVIRFKVTAGMLASNNGVL |
| (6X-His) | | | YTNYPISGCFDREKFHEVRFSSDFSKDTYCDVTVTTPGAY |
| | | | EYYVEYEPINQENSDNQPEPKRSSRSGYFVVEPRLYIDSP |
| | | | VKPSEPSAEKKEVQEHAPAKVLLPLDGLVIESAVGKWLGP |
| | | | LDTWDAHLQHMKAAGYNMIHFVPLQVRGASNSPYSIHDQ |
| | | | TAFSDDLFNEQDVKKSSEEKAQLVNKQLHRIQDEYGIMCL |
| | | | SDVVWNHTSFDSVWLQDHPESGYNLDNSPHLQAAFELD |
| | | | TALINLVDDFSKYSLPTTLKSGQDLDAVMNVIKNNVFPDLK |
| | | | LWEYKVIDVVSEKKKLHEALLAAPKETAQSQVFENANLSG |
| | | | LASDQQAQLLAEHGIADRQSGSRYGKTVDIDVAVAFIKAL |
| | | | GGVASGEQVSEDVAAKLVDIYGKVLDVYNLPFYKEYDED |
| | | | VRIALENTKNRIEYTRLADHGPKWGEITKEKPIVDNYFTRL |
| | | | PKNDKTKGHSEGSLALANNGWIWNADPMQDFAGPQSAA |
| | | | YLRREVICWGDCVKLRYGSGADDNPYLWKHMKDYTEQT |
| | | | AKYFQGIRIDNCHSTPIHVAQYLLDAARKIRPDLYVVAELFT |
| | | | GSEDSDVTFVSKLGINSLIREAMQAWDAAELSRLVHRHG |
| | | | GKAIGSMDQALTTEQINYSLNGKEEEPALLVPVLQGSVPH |
| | | | ALFMDCTHDNETPNEKRTPQDTLPNAALVAFSGSAIGSVK |
| | | | GYDDIYPRLLDIVNETRLYDLPKPDANVGINKVKPVLQKLH |
| | | | TEMIMDGYIECHVHHEGDYIIVHRQHPELHTGYLLVAHTAF |
| | | | PGRQNNRGSISPIKLRGTDVDIMFSATLDIESRENLSDEKT |
| | | | LRGLKSKIVSLSAPTIKSLSDNKGRYTEITVPASFPSGSIMV |
| | | | LKTWIQDNPAESYDLISSCSDDVFKNMTSLDMNVVLHRCE |
| | | | GEERDATGEGVYNIPGFGGLPYAGLEGFVSVLKPIIAHND |
| | | | LGHPFCAHLREGTWAMDFIVDRLNRYIGNYPNLKRLKDW |
| | | | FASRFSEVKGLPDFLLPKYFALIIRTAYLKARAHAVSLMSPL |
| | | | VREGDRFIQSLALCSVQMYGIVPSSGLHPTEVTPSMAAGL |
| | | | PHFTHSYMRTWGRDVFIALRGLFLTTGNYDAAKRHIVSFA |
| | | | SSLKHGLIPNLLDAVRHPRYNSRDSVWWFLQAVQDYYNM |
| | | | APDGKSILQEKVSRRFPKNDTFVPPEEGFKYSCTVAEIIQE |
| | | | IFERHAAGIHFREHNAGEQLDRQMLDEGFNIDIDVDWESG |
| | | | TLIGGNRFNCGTWQDKMGESVKAGNKGLPATPRDGAPIE |
| | | | ITGLLKSTLRWVVELHERGEFQWTSAKDKSGNERSYKDW |
| | | | NDLLQTNFERVYYVPLDEKEDSQYDVLSQIVNRRGIYKDV |
| | | | YKSKEPYTDYQFRSNVPVAMVVAPELFTPAKAMGSLELA |
| | | | RSNLLGPLGMATLDPSDNEYHPDYHNSDDSDNKAVAKG |
| | | | WNYHQGPEWVWQTGYFLRAYLYFSLKTGEDQSKREKVM |
| | | | DIQRILLAHKREIESSPWAGLPELTNRNGNPCWDSCPTQA |
| | | | CYDDEVVLIQSQNTSFNHRSPATSDQESAAINFFLFCANLI |
| | | | SEEPMNNDQVIPLETHKLGLELRPHPQRGRGVFATCALK |
| | | | ARTLVEISPILLVNHDEYSAHGQYTILDHYTYRWEGGYALA |
| | | | LGLGSMFNHAKNPNVGFVRDIPNAVIRYFTLRAIEPAEELC |
| | | | ISYGDHLWFEDTDAKSEASIDSSEDEWFGKIMVDDEEHH |
| | | | HHHH |
| CCO28440.1 | Thanatephorus | 350 | MPVPITKARSGSTSEAAPGHPKAGAGTTRGQRPPPVQLP |
| | cucumeris (strain | | HERYADAPKTPGPKTPADEALDFFESTERGDARIQVYELR |
| | AG1-IB/isolate | | LEPDGGPNREAAYTRLPPAYKPYILRVTIDAGSPASRNGV |
| | 7/3/14) | | FKTNFPLDGGKFSRDHFVERKLPTDFSKPIKIDLPISHAGA |
| | | | FVYWLEYDGSQGRIKAREGYFNIDPILTTYRRTPVIDHKTL |
| | | | AVQAQTGFVEPRESVNIPLDGIAMLTVVSKWMGPLPEWE |
| | | | QHFAEARTRGYNMLHYTPLQQRGESKSPYSIADQMAFDS |
| | | | GLFEDNYDGSKEEGTQRVKDILKLAKKQYGLMSLTDVVLN |
| | | | HTANNRFSPYNCPHLTPALELDNAIIDFSTDLASRGLPTLIT |
| | | | SQKDIDTLIDALKEHIAKLNLWQYYVLNGQSEKEAVADAIA |
| | | | ASKITPWTGPDIVGKSVAEIADIVRGEGNLEHVGEFAERF |
| | | | MITVNPSVAAGIVKTAFVDLGDATPAAYGEAWGRVVDVLN |
| | | | VDRYKEWEEDTKVAIENIINRVKWTRLDENGPKVGEINKT |
| | | | MPLVDTYFTRIPKNSRTAKHDPRALAVANNGWIWAADPLA |
| | | | NFALRPSKAYLRREVIVWGDCVKLRYGDGPSDNPWLWK |
| | | | HMTEYVQSLAVLFDGFRIDNCHSTPLHVGVALLDAARAVN |
| | | | PDLYVCAELFTGSEEMDVHFVSRLGINSLIREAMNGWDPK |
| | | | DFARLLHRYGLGKPIGSMDESCLTSHEDLPPPTGKGATRP |
| | | | AAVTPIRGSAPHALMYDLTHDNESPLHKRSAEDALSTGAL |
| | | | VTFGLSATGSNKGFDDLYPKLLDLVGDNRKYETSDGGKV |
| | | | DRGIAKVKKVLNQLHTEMVLAGFKEGHVHQEGDYIAIHRV |
| | | | HPATQKGYFLIAHTAFGSTKGSKARGDVNPISLKSTKARFI |
| | | | LGASIDIPSYELEASKTTISGLPSTLKELAEVPPKVTDDGCE |
| | | | LIVPEEFPPGSILLYETQLVGVDPNLESACAEGADEAFAEL |
| | | | DLVDLNVVLHRCSGEERDATNGEIGTYNIGGLGDLVYCGL |
| | | | EGWMHPLRHVMNHNDLGHPLCENLRQGDWALGYVQSR |
| | | | LEKQTPTFPRLAKPAAWFKSRFDLIRAQVPGFLRPKYFAIV |
| | | | IFSAYKVARRIAIEQCSEFVVSGHDFTQNLALCQIQMHGLV |
| | | | QSASIDPGKVVPSLAAGLPHFAGGWARTWGRDVFISLRG |
| | | | LFLATGNYAAARAHILAFCATLKHGLIPNLLDALRTPRYNS |
| | | | RDSPWWMVQNIQDYCNMAPEGLALLEAPVKRRFPQDDT |
| | | | FVPWDDPRAYAYSSSVAEIIQEILQRHAKGISFREHNAGP |
| | | | NLDMQMSDEGFNIEVKVDWETGFTLGGNWKNCGTWMD |
| | | | KMGESQKAGTKGTPGTPRDGAPVEITGLLFSTLRWLDGL |
| | | | GSKFPFKGVQATIDGQEKLVTYREWATLIQNAFERYYYVP |
| | | | LNPVEDPDYVVDPRIINQRGIYKDVYGSGAGREWSDYQL |
| | | | RCNFPIAMVVAPELFNPEHALGALKIADKRLRGPLGMKTL |
| | | | DEGDSQYRGYYDNSNDSDDASIAKGLNYHQGPEWGWPL |
| | | | GYFLRAYLKFDQIAGAGKNDQAEMDAPTAAEDQTPSTPE |
| | | | LRRTYLSLLSHNAIVELVLSLNADVTSSLFPSDLAGEVRRM |
| | | | QSSSATDDVDGEGSPDPQPAPPPRAPSVPPAGGPRTRA |
| | | | GAARAYAAALASKDTPSTRPSPAPGGVSFPQTPEPQQPQ |
| | | | QSPPPFAPISQPYPSLIVHLPGGSPPIPSPTTPTNHASTAA |
| | | | NPLPGIGPVRNTPAKKTRTGVIGGYNPITSIGPSSEGLPSY |
| | | | EEMIVLALMEGQDDEGVAPKDVFAWMSARWPLNANFRP |
| | | | SASQALQKAYKRGRLEKVGTKYKLNPNWHGGATTNRTT |
| | | | RRPQSMAEVPGNYAWAPPPVPPVVSVPPDPRTRHSITPN |
| | | | PSTEPPASQQTQTEFDAQAQVASLLKALQEAGPGPSNPN |
| | | | PNSSGPEQNQPPNVEEGVHTHLPHMSIPGAADGARPIQS |
| | | | SPETAVIVPTTGSPSALGPSQSPVQISQLPASMPPLPAPV |
| | | | QPGLPTPPIQAIMSPVQSQNGILPLQNPTSPPIQNGVSSQ |
| | | | PTTNTSLSQPQPSQPPQPQVLHQPHVPPQNSAIPQPPHP |
| | | | MPPGVPMTYPSFHTTPSALQASLVTLASQLANMSKTNQG |
| | | | A |
| CCO28440.1 | Thanatephorus | 351 | MPVPITKARSGSTSEAAPGHPKAGAGTTRGQRPPPVQLP |
| (6X-His) | cucumeris (strain | | HERYADAPKTPGPKTPADEALDFFESTERGDARIQVYELR |
| | AG1-IB/isolate | | LEPDGGPNREAAYTRLPPAYKPYILRVTIDAGSPASRNGV |
| | 7/3/14) | | FKTNFPLDGGKFSRDHFVERKLPTDFSKPIKIDLPISHAGA |
| | | | FVYWLEYDGSQGRIKAREGYFNIDPILTTYRRTPVIDHKTL |
| | | | AVQAQTGFVEPRESVNIPLDGIAMLTVVSKWMGPLPEWE |
| | | | QHFAEARTRGYNMLHYTPLQQRGESKSPYSIADQMAFDS |
| | | | GLFEDNYDGSKEEGTQRVKDILKLAKKQYGLMSLTDVVLN |
| | | | HTANNRFSPYNCPHLTPALELDNAIIDFSTDLASRGLPTLIT |
| | | | SQKDIDTLIDALKEHIAKLNLWQYYVLNGQSEKEAVADAIA |
| | | | ASKITPWTGPDIVGKSVAEIADIVRGEGNLEHVGEFAERF |
| | | | MITVNPSVAAGIVKTAFVDLGDATPAAYGEAWGRVVDVLN |
| | | | VDRYKEWEEDTKVAIENIINRVKWTRLDENGPKVGEINKT |
| | | | MPLVDTYFTRIPKNSRTAKHDPRALAVANNGWIWAADPLA |
| | | | NFALRPSKAYLRREVIVWGDCVKLRYGDGPSDNPWLWK |
| | | | HMTEYVQSLAVLFDGFRIDNCHSTPLHVGVALLDAARAVN |
| | | | PDLYVCAELFTGSEEMDVHFVSRLGINSLIREAMNGWDPK |
| | | | DFARLLHRYGLGKPIGSMDESCLTSHEDLPPPTGKGATRP |
| | | | AAVTPIRGSAPHALMYDLTHDNESPLHKRSAEDALSTGAL |
| | | | VTFGLSATGSNKGFDDLYPKLLDLVGDNRKYETSDGGKV |
| | | | DRGIAKVKKVLNQLHTEMVLAGFKEGHVHQEGDYIAIHRV |
| | | | HPATQKGYFLIAHTAFGSTKGSKARGDVNPISLKSTKARFI |
| | | | LGASIDIPSYELEASKTTISGLPSTLKELAEVPPKVTDDGCE |
| | | | LIVPEEFPPGSILLYETQLVGVDPNLESACAEGADEAFAEL |
| | | | DLVDLNVVLHRCSGEERDATNGEIGTYNIGGLGDLVYCGL |
| | | | EGWMHPLRHVMNHNDLGHPLCENLRQGDWALGYVQSR |
| | | | LEKQTPTFPRLAKPAAWFKSRFDLIRAQVPGFLRPKYFAIV |
| | | | IFSAYKVARRIAIEQCSEFVVSGHDFTQNLALCQIQMHGLV |
| | | | QSASIDPGKVVPSLAAGLPHFAGGWARTWGRDVFISLRG |
| | | | LFLATGNYAAARAHILAFCATLKHGLIPNLLDALRTPRYNS |
| | | | RDSPWWMVQNIQDYCNMAPEGLALLEAPVKRRFPQDDT |
| | | | FVPWDDPRAYAYSSSVAEIIQEILQRHAKGISFREHNAGP |
| | | | NLDMQMSDEGFNIEVKVDWETGFTLGGNWKNCGTWMD |
| | | | KMGESQKAGTKGTPGTPRDGAPVEITGLLFSTLRWLDGL |
| | | | GSKFPFKGVQATIDGQEKLVTYREWATLIQNAFERYYYVP |
| | | | LNPVEDPDYVVDPRIINQRGIYKDVYGSGAGREWSDYQL |
| | | | RCNFPIAMVVAPELFNPEHALGALKIADKRLRGPLGMKTL |
| | | | DEGDSQYRGYYDNSNDSDDASIAKGLNYHQGPEWGWPL |
| | | | GYFLRAYLKFDQIAGAGKNDQAEMDAPTAAEDQTPSTPE |
| | | | LRRTYLSLLSHNAIVELVLSLNADVTSSLFPSDLAGEVRRM |
| | | | QSSSATDDVDGEGSPDPQPAPPPRAPSVPPAGGPRTRA |
| | | | GAARAYAAALASKDTPSTRPSPAPGGVSFPQTPEPQQPQ |
| | | | QSPPPFAPISQPYPSLIVHLPGGSPPIPSPTTPTNHASTAA |
| | | | NPLPGIGPVRNTPAKKTRTGVIGGYNPITSIGPSSEGLPSY |
| | | | EEMIVLALMEGQDDEGVAPKDVFAWMSARWPLNANFRP |
| | | | SASQALQKAYKRGRLEKVGTKYKLNPNWHGGATTNRTT |
| | | | RRPQSMAEVPGNYAWAPPPVPPVVSVPPDPRTRHSITPN |
| | | | PSTEPPASQQTQTEFDAQAQVASLLKALQEAGPGPSNPN |
| | | | PNSSGPEQNQPPNVEEGVHTHLPHMSIPGAADGARPIQS |
| | | | SPETAVIVPTTGSPSALGPSQSPVQISQLPASMPPLPAPV |
| | | | QPGLPTPPIQAIMSPVQSQNGILPLQNPTSPPIQNGVSSQ |
| | | | PTTNTSLSQPQPSQPPQPQVLHQPHVPPQNSAIPQPPHP |
| | | | MPPGVPMTYPSFHTTPSALQASLVTLASQLANMSKTNQG |
| | | | AHHHHHH |
| KKF96931.1 | Ceratocystis | 352 | MAKTSDETSFASSMPNFSRNIEIDITIPHAGAYAYYITYAGL |
| | fimbriata f. sp. | | PPLAEVLESIDTAEPEVATHKTDLFYIDVAPYLTINSSSLPL |
| | platani | | DGLSVFTVLSKFMGKYPKDWSSHFHGISERGYNMVHFTP |
| | | | LQVRGSSNSPYSLFDQLAWDSQFFPNGESDIAKLVDDLE |
| | | | KNYGLLSLTDVVLNHTAHNSKWLEEHPEAGYNLTTAPWL |
| | | | ESAYILDTKLLQLSSKLTEKALPTVLNDEKDLLKIMDAIKTD |
| | | | VIFDMRLWEFYTLDIEANATAAVAAWKSGKYSFPEEKDIE |
| | | | MINKPLKEQAKYLTDNGLSGNDRMGSRFRRHIDPEISGGL |
| | | | LALHFGRFESDSTNGADEGAVKTKISTILDAANLPFYKEYD |
| | | | AEVADILQQLFNRIKYMRLEENGPKLGEITAENPLIETYFTR |
| | | | LPKNETTAQHSEDDLALANNGWVWGGNALIDNAGPNSR |
| | | | VYLRREVIVWGDCVKLRYGSSPEDSPYLWDRMTKYVRLL |
| | | | TKYFAGLRIDNCHSTPIHVAEHILDEARRIRPNVYVVAELFS |
| | | | GSEETDYVFVKRLGLSSLIREAMQAWSTGELNGTKSTEST |
| | | | SEHTRNNSITKSRELIRNIKPVPVQALFMDCTHDNETPFQK |
| | | | RDARDTLPNAALVSMCASASGSVMGYDEIYPKIIDLVGET |
| | | | RLYVSEASSSPSVKIGAGKNGIAGVKKLLNQIHTLMGKDG |
| | | | YDETHVHHEDQYVTVHRVHPKSRKGYFLIAHTAFPGYGN |
| | | | GNGAFNPVHLTGTKAKHLGSWMLEVDTSEEALAQVQND |
| | | | KTYLNGLPSQVVDLSSVKLDEAAGAFNELDLVDLNFVLYR |
| | | | CEAEERSTSNGQDERFDALRDIPSFLIPRYFALVIRTAYRA |
| | | | SWDRAMNLMSPGVQEGQWFMQSLAMVSVQQTGIVPSA |
| | | | SLYPKTQVPSLAAGLPHFAVEWARCWGRDVMISLRGLYL |
| | | | GTGRFDEAKEHIKAFGSVMKHGMIPNLLGSGGNPRYNAR |
| | | | DSIWFFLQCIQEYTKIVPDGISILNEKVKRRFLPYDDTWFAV |
| | | | DDSRAYSNESTIAEIIQEALQRHAAGISFREANAGSAIDSQ |
| | | | MSDEGFNIDIKVDWENGFIFGGNQHNCGTWMDKMGESV |
| | | | KARSKGVPGTPRDGAAVEITGMLYSTLKWVSELNATGKF |
| | | | AKPGVEKADGSAISFADWAALVKTNFERCYFVPKDSVDD |
| | | | SKYDVNPSVINRRGIYKDLYRSGKEYEDYQLRPNFPIAMC |
| | | | VAPDLFEPAHALSALFVAESVLRGPTGMATLDPTDLNYRP |
| | | | YYINSEDSTDFATAKGRNYHQGPEWLWPTGFFLRALLKF |
| | | | DLMRRGDGAAARTESFQQITQRLIGCKKMIRESPWAGLA |
| | | | ELTQKNGEFCGDSCPTQAWSASCLIDLYMDAAMEHHSH |
| | | | DMSIPIHRK |
| KKF96931.1 | Ceratocystis | 353 | MAKTSDETSFASSMPNFSRNIEIDITIPHAGAYAYYITYAGL |
| (6X-His) | fimbriata f. sp. | | PPLAEVLESIDTAEPEVATHKTDLFYIDVAPYLTINSSSLPL |
| | platani | | DGLSVFTVLSKFMGKYPKDWSSHFHGISERGYNMVHFTP |
| | | | LQVRGSSNSPYSLFDQLAWDSQFFPNGESDIAKLVDDLE |
| | | | KNYGLLSLTDVVLNHTAHNSKWLEEHPEAGYNLTTAPWL |
| | | | ESAYILDTKLLQLSSKLTEKALPTVLNDEKDLLKIMDAIKTD |
| | | | VIFDMRLWEFYTLDIEANATAAVAAWKSGKYSFPEEKDIE |
| | | | MINKPLKEQAKYLTDNGLSGNDRMGSRFRRHIDPEISGGL |
| | | | LALHFGRFESDSTNGADEGAVKTKISTILDAANLPFYKEYD |
| | | | AEVADILQQLFNRIKYMRLEENGPKLGEITAENPLIETYFTR |
| | | | LPKNETTAQHSEDDLALANNGWVWGGNALIDNAGPNSR |
| | | | VYLRREVIVWGDCVKLRYGSSPEDSPYLWDRMTKYVRLL |
| | | | TKYFAGLRIDNCHSTPIHVAEHILDEARRIRPNVYVVAELFS |
| | | | GSEETDYVFVKRLGLSSLIREAMQAWSTGELNGTKSTEST |
| | | | SEHTRNNSITKSRELIRNIKPVPVQALFMDCTHDNETPFQK |
| | | | RDARDTLPNAALVSMCASASGSVMGYDEIYPKIIDLVGET |
| | | | RLYVSEASSSPSVKIGAGKNGIAGVKKLLNQIHTLMGKDG |
| | | | YDETHVHHEDQYVTVHRVHPKSRKGYFLIAHTAFPGYGN |
| | | | GNGAFNPVHLTGTKAKHLGSWMLEVDTSEEALAQVQND |
| | | | KTYLNGLPSQVVDLSSVKLDEAAGAFNELDLVDLNFVLYR |
| | | | CEAEERSTSNGQDERFDALRDIPSFLIPRYFALVIRTAYRA |
| | | | SWDRAMNLMSPGVQEGQWFMQSLAMVSVQQTGIVPSA |
| | | | SLYPKTQVPSLAAGLPHFAVEWARCWGRDVMISLRGLYL |
| | | | GTGRFDEAKEHIKAFGSVMKHGMIPNLLGSGGNPRYNAR |
| | | | DSIWFFLQCIQEYTKIVPDGISILNEKVKRRFLPYDDTWFAV |
| | | | DDSRAYSNESTIAEIIQEALQRHAAGISFREANAGSAIDSQ |
| | | | MSDEGFNIDIKVDWENGFIFGGNQHNCGTWMDKMGESV |
| | | | KARSKGVPGTPRDGAAVEITGMLYSTLKWVSELNATGKF |
| | | | AKPGVEKADGSAISFADWAALVKTNFERCYFVPKDSVDD |
| | | | SKYDVNPSVINRRGIYKDLYRSGKEYEDYQLRPNFPIAMC |
| | | | VAPDLFEPAHALSALFVAESVLRGPTGMATLDPTDLNYRP |
| | | | YYINSEDSTDFATAKGRNYHQGPEWLWPTGFFLRALLKF |
| | | | DLMRRGDGAAARTESFQQITQRLIGCKKMIRESPWAGLA |
| | | | ELTQKNGEFCGDSCPTQAWSASCLIDLYMDAAMEHHSH |
| | | | DMSIPIHRKHHHHHH |
| EBA46363.1 | Burkholderia | 354 | MRIRHSFLCVFMLAAGSHARATEFNASFLSIDGRNDVDLS |
| | pseudomallei 305 | | QFAQADYTLPGTYLLDVQVNDVFFGLQSIEFVAHDDGQG |
| | | | ARACVAPELVAQFGLKKSLVENLPRTMGGRCADLASLDG |
| | | | VTIRYQKGEGRLKITIAQAALEFADASYLPPERWSDGVDG |
| | | | AMLDYRLLANANHAFGRGAQQNNSVQAYGTIGANRGAW |
| | | | RFRGDYQAQTRAGGAVYAERAFRFNQLYAYRALPSIRST |
| | | | LSFGEIYVDSDIFSTFSMSGVAMKSDDRMLPPSMRGYAPL |
| | | | VTGVARTNAIVKVMQDSRVLYMTKVSPGAFALSNLNTSV |
| | | | QGTLDVVVEEEDGTVQRFQVATASVPFLAREGQLRYKTAI |
| | | | GQPRTFGGAGITPWFGFAEAAYGLPFDVTVYGGLIAASG |
| | | | YTSVAFGVGRDFGRFGALSADVTHARATLWWNGRTKRG |
| | | | NSYRINYSKHVDALDADVRFFGYRFSERDYTNFQQFSGD |
| | | | PTASGLANGKQRYSAMLSKRFGDTSTYFSYDQTTYWARP |
| | | | SDRRIGVTLTRAFSLGALKSVNLGFSAFRTQGAGGGGNQ |
| | | | VSLTATLPLGERQTLTSSVSAGEGGTSVNAGYLYDGANG |
| | | | RTYQLYGGTTDGRASANASLRQRTPSYQLTAQASTVANA |
| | | | YASASLEVDGSFVATRYGVTAHANGNAGDTRLLVSTDGV |
| | | | PGVPLSGSYARTNARGYAVIDGVSPYNVYDATVSVEKLGL |
| | | | DTDVTNPIQRTVLTDGAIGYIRFNAARGRNVFVTLTGDGG |
| | | | APVPFGASVQDAATGKELGIVGEAGAAYLTQVQPRAKLV |
| | | | VRAGAKTICTPAALPDTLQLDGTPAPVACEAAAQHASR |
| EBA46363.1 | Burkholderia | 355 | TEFNASFLSIDGRNDVDLSQFAQADYTLPGTYLLDVQVND |
| (6X-His) | pseudomallei 305 | | VFFGLQSIEFVAHDDGQGARACVAPELVAQFGLKKSLVEN |
| | | | LPRTMGGRCADLASLDGVTIRYQKGEGRLKITIAQAALEFA |
| | | | DASYLPPERWSDGVDGAMLDYRLLANANHAFGRGAQQN |
| | | | NSVQAYGTIGANRGAWRFRGDYQAQTRAGGAVYAERAF |
| | | | RFNQLYAYRALPSIRSTLSFGEIYVDSDIFSTFSMSGVAMK |
| | | | SDDRMLPPSMRGYAPLVTGVARTNAIVKVMQDSRVLYMT |
| | | | KVSPGAFALSNLNTSVQGTLDVVVEEEDGTVQRFQVATA |
| | | | SVPFLAREGQLRYKTAIGQPRTFGGAGITPWFGFAEAAYG |
| | | | LPFDVTVYGGLIAASGYTSVAFGVGRDFGRFGALSADVTH |
| | | | ARATLWWNGRTKRGNSYRINYSKHVDALDADVRFFGYRF |
| | | | SERDYTNFQQFSGDPTASGLANGKQRYSAMLSKRFGDT |
| | | | STYFSYDQTTYWARPSDRRIGVTLTRAFSLGALKSVNLGF |
| | | | SAFRTQGAGGGGNQVSLTATLPLGERQTLTSSVSAGEGG |
| | | | TSVNAGYLYDGANGRTYQLYGGTTDGRASANASLRQRT |
| | | | PSYQLTAQASTVANAYASASLEVDGSFVATRYGVTAHAN |
| | | | GNAGDTRLLVSTDGVPGVPLSGSYARTNARGYAVIDGVS |
| | | | PYNVYDATVSVEKLGLDTDVTNPIQRTVLTDGAIGYIRFNA |
| | | | ARGRNVFVTLTGDGGAPVPFGASVQDAATGKELGIVGEA |
| | | | GAAYLTQVQPRAKLVVRAGAKTICTPAALPDTLQLDGTPA |
| | | | PVACEAAAQHASRHHHHHH |
| ATA77697.1 | Capnocytophaga | 356 | MSYLTFKKKELINLEYSLDREFLGTNRGGGYSSSTLVFCN |
| | canimorsus | | TRKYHGLLVVPLENFRGQNHVLLSSLDETIIQHGRDFNFA |
| | | | VHQYPETYEPRGHKYIVDVSYEKAFTVIYQVGGVTLKKEIL |
| | | | MVHNEPQVLVRYTLLEAHSPTKLRLNPLLAFRDIHSLSKVN |
| | | | DLANKNIEKIEGGIKTKLYPDFPYLHLQLSQDTDFEYQPNW |
| | | | NCNIFYKKEKERGYDYTEDLLTLGYFELNIKKGESVVFSAS |
| | | | TEENNSKQLKQKFTRLINQRRERNSFEECLNYSATQFLIQ |
| | | | KAGEIRVKEGYHWYNSNTRDTFIALPGIALAENTQKTFDE |
| | | | VLTSVKKYFSNGLFEKTVSTHVAERQYDADTSLWFFWTIQ |
| | | | QYEKYTKKTPKQLWKDFGELMRTILETYKNGQHKSIRMD |
| | | | NNGLIWSEDISRPLTWVDAQFDGGAIVPRNGYVVEVNAL |
| | | | WYNAVCYALALAEASKDKDFLKEWSDLPMMIAENFNQTF |
| | | | WISDKKYLADYVNYAHQNRDVRPNQLIPCALAYSPLDERS |
| | | | KKRVLDIVRQELLTPRGLRSLSPKNKSYESVSIGNVYDRD |
| | | | RATFNGSVHPWFIGFYIEAHLKIYGKTYIPDAEAILADFEEE |
| | | | LSGHGVSTISAIYDGNPPFRSRGCISKARNVAEILRAKQLT |
| | | | MNYES |
| ATA77697.1 | Capnocytophaga | 357 | MSYLTFKKKELINLEYSLDREFLGTNRGGGYSSSTLVFCN |
| (6X-His) | canimorsus | | TRKYHGLLVVPLENFRGQNHVLLSSLDETIIQHGRDFNFA |
| | | | VHQYPETYEPRGHKYIVDVSYEKAFTVIYQVGGVTLKKEIL |
| | | | MVHNEPQVLVRYTLLEAHSPTKLRLNPLLAFRDIHSLSKVN |
| | | | DLANKNIEKIEGGIKTKLYPDFPYLHLQLSQDTDFEYQPNW |
| | | | NCNIFYKKEKERGYDYTEDLLTLGYFELNIKKGESVVFSAS |
| | | | TEENNSKQLKQKFTRLINQRRERNSFEECLNYSATQFLIQ |
| | | | KAGEIRVKEGYHWYNSNTRDTFIALPGIALAENTQKTFDE |
| | | | VLTSVKKYFSNGLFEKTVSTHVAERQYDADTSLWFFWTIQ |
| | | | QYEKYTKKTPKQLWKDFGELMRTILETYKNGQHKSIRMD |
| | | | NNGLIWSEDISRPLTWVDAQFDGGAIVPRNGYVVEVNAL |
| | | | WYNAVCYALALAEASKDKDFLKEWSDLPMMIAENFNQTF |
| | | | WISDKKYLADYVNYAHQNRDVRPNQLIPCALAYSPLDERS |
| | | | KKRVLDIVRQELLTPRGLRSLSPKNKSYESVSIGNVYDRD |
| | | | RATFNGSVHPWFIGFYIEAHLKIYGKTYIPDAEAILADFEEE |
| | | | LSGHGVSTISAIYDGNPPFRSRGCISKARNVAEILRAKQLT |
| | | | MNYESHHHHHH |
| ALA59081.1 | Nitrospira | 358 | MAFSIARAVVIKHGNVFFLAPEEGEIPFTSGHGFGLYYHDC |
| | moscoviensis | | RYLNGYELLVGDVHPEPLGARASEGARAMFRLTTPDFRM |
| | | | ADGRAVPRETVGLKWERVIDSEQCLLQERLEIQNFGHEP |
| | | | VEFPLAMRFRAGFEDVFEIRGLLREVKGKLYRPGWRDKC |
| | | | LCFSYEGGDHVTRSLAVHFDPLPPDVKGSAVQFHLRLQP |
| | | | NEINVIRIVLAIAESDRVPATVCAQVNIRRDLPTVVRVMRSE |
| | | | CEEWMMRETEVHTDSEAVNRIMKRSLMDLVMLRTTIGGD |
| | | | EFFAAGVPWYATLFGRDSLVTSLQTLAYGAETAADSLRLL |
| | | | ARYQGDRVSEWRDEEPGKILHELRVGELARMNKIPHTPY |
| | | | YGTIDATPLFIILMSRYADWTGDLSLFTELRPHVERALEWI |
| | | | DRYGDAAGDGYVSYLGHSDKGLLHQGWKDSGDAIVTAD |
| | | | GRFAEPPIALVEVQGYVYLAKRSIADLYERVGEPDRAALL |
| | | | REEARELRRRFNRDFWMEDEGCYALALETGGRPCRVLS |
| | | | SNAGQALWTGIVDEEKAGRVVDRLMRPDLFSGWGVRTL |
| | | | SYKERRYNPMGYHLGTVWPHDNSLIAAGFRRYGFDEAA |
| | | | GRIFAGLVDAAREFEDFRLPELFTGFGREEYGVPVRYPVA |
| | | | CHPQAWAAGSVPFMIETLLGLEPEALEHRLRVVRPFLPDF |
| | | | IGRLDLRHLQVGKGAADLRFSRKADGRVDVEVLNIAGTLT |
| | | | VEVEKQ |
| ALA59081.1 | Nitrospira | 359 | MAFSIARAVVIKHGNVFFLAPEEGEIPFTSGHGFGLYYHDC |
| (6X-His) | moscoviensis | | RYLNGYELLVGDVHPEPLGARASEGARAMFRLTTPDFRM |
| | | | ADGRAVPRETVGLKWERVIDSEQCLLQERLEIQNFGHEP |
| | | | VEFPLAMRFRAGFEDVFEIRGLLREVKGKLYRPGWRDKC |
| | | | LCFSYEGGDHVTRSLAVHFDPLPPDVKGSAVQFHLRLQP |
| | | | NEINVIRIVLAIAESDRVPATVCAQVNIRRDLPTVVRVMRSE |
| | | | CEEWMMRETEVHTDSEAVNRIMKRSLMDLVMLRTTIGGD |
| | | | EFFAAGVPWYATLFGRDSLVTSLQTLAYGAETAADSLRLL |
| | | | ARYQGDRVSEWRDEEPGKILHELRVGELARMNKIPHTPY |
| | | | YGTIDATPLFIILMSRYADWTGDLSLFTELRPHVERALEWI |
| | | | DRYGDAAGDGYVSYLGHSDKGLLHQGWKDSGDAIVTAD |
| | | | GRFAEPPIALVEVQGYVYLAKRSIADLYERVGEPDRAALL |
| | | | REEARELRRRFNRDFWMEDEGCYALALETGGRPCRVLS |
| | | | SNAGQALWTGIVDEEKAGRVVDRLMRPDLFSGWGVRTL |
| | | | SYKERRYNPMGYHLGTVWPHDNSLIAAGFRRYGFDEAA |
| | | | GRIFAGLVDAAREFEDFRLPELFTGFGREEYGVPVRYPVA |
| | | | CHPQAWAAGSVPFMIETLLGLEPEALEHRLRVVRPFLPDF |
| | | | IGRLDLRHLQVGKGAADLRFSRKADGRVDVEVLNIAGTLT |
| | | | VEVEKQHHHHHH |
| BAT71421.1 | Thermosulfidi- | 360 | MSEGNRCEKIQVDGEWYILCSSVPLTTRKLTLKGDRAFVI |
| | bacter | | TDAYGDVPVAYPSELGYYWRDTRFLSGFEMYLNGARPVL |
| | takaii (strain | | LSSDIDADSKCIIVEMTNTDFRVGNKIVHRTSLYLSKTITFE |
| | DSM 17441/ | | GDDLVQVVVLRNLSLFDLPCDLQFIMEADFKDIFEVRGSE |
| | JCM 13301/ | | RDKRGESLEPIIGKGYVEYRYKGLDGLLRSCNVQFSPSDF |
| | NBRC 103674/ | | CFEGNAARFKFVLEPKKEIEIVIRASCKIEDSLIFCTKNTPKP |
| | ABI70S6) | | GFESHVDFSSSNQLLSSVVGRSLKDLEMMLSDFDGHAIP |
| | | | MAGIPWYCALFGRDSIITALELLPWYPELARNVLAVLSRYQ |
| | | | ARDFDDFTDSEPGKILHELREGEMARLREIPFVPYYGTAD |
| | | | ATLLYITLAAEYVKTTGDLDFLKSIWENINLATKWMEHYGDI |
| | | | NGDGFVEYMSRSPVGLRNQGWKDSHDSIHHVDGALAEP |
| | | | PISVVEVQGYKYQALKALSFLSKCMGNVGEAERFDRRAQ |
| | | | ELASKLEGVFWMPSKGFYALALDKNGKRCEIISSNAGHLL |
| | | | FACAVSRERAAKVAERLLSDEMFTGYGIRTLSRHEVRYNP |
| | | | MSYHNGSVWPHDCAIICRGLVNYGFKREALKIFRGILEAV |
| | | | ALFPDYRVPELFCGFPRDSEKPPVPYPVACSPQAWSSAS |
| | | | VIFMLTSLLGIEVDAFNKILTFNNPVLPSEIQELEIKDWFTPF |
| | | | FGEFSFKFVNVGERVAVETLKKPHDWKVLVVVG |
| BAT71421.1 | Thermosulfidi- | 361 | MSEGNRCEKIQVDGEWYILCSSVPLTTRKLTLKGDRAFVI |
| (6X-His) | bacter | | TDAYGDVPVAYPSELGYYWRDTRFLSGFEMYLNGARPVL |
| | takaii (strain | | LSSDIDADSKCIIVEMTNTDFRVGNKIVHRTSLYLSKTITFE |
| | DSM 17441/ | | GDDLVQVVVLRNLSLFDLPCDLQFIMEADFKDIFEVRGSE |
| | JCM 13301/ | | RDKRGESLEPIIGKGYVEYRYKGLDGLLRSCNVQFSPSDF |
| | NBRC 103674/ | | CFEGNAARFKFVLEPKKEIEIVIRASCKIEDSLIFCTKNTPKP |
| | ABI70S6) | | GFESHVDFSSSNQLLSSVVGRSLKDLEMMLSDFDGHAIP |
| | | | MAGIPWYCALFGRDSIITALELLPWYPELARNVLAVLSRYQ |
| | | | ARDFDDFTDSEPGKILHELREGEMARLREIPFVPYYGTAD |
| | | | ATLLYITLAAEYVKTTGDLDFLKSIWENINLATKWMEHYGDI |
| | | | NGDGFVEYMSRSPVGLRNQGWKDSHDSIHHVDGALAEP |
| | | | PISVVEVQGYKYQALKALSFLSKCMGNVGEAERFDRRAQ |
| | | | ELASKLEGVFWMPSKGFYALALDKNGKRCEIISSNAGHLL |
| | | | FACAVSRERAAKVAERLLSDEMFTGYGIRTLSRHEVRYNP |
| | | | MSYHNGSVWPHDCAIICRGLVNYGFKREALKIFRGILEAV |
| | | | ALFPDYRVPELFCGFPRDSEKPPVPYPVACSPQAWSSAS |
| | | | VIFMLTSLLGIEVDAFNKILTFNNPVLPSEIQELEIKDWFTPF |
| | | | FGEFSFKFVNVGERVAVETLKKPHDWKVLVVVGHHHHHH |
| CED91394.1 | Actinomyces | 362 | MIVNAPAQAIAAPDGSMGDAPVQGVYQSDVRALRLRALS |
| | succiniciruminis | | VTGSALEHLSVERCGAAASRHRALARGLDRGGADPRSIIT |
| | | | TDRTLVPADDGTVLTETLTVRIAQDRPVSTTLTLELAADLS |
| | | | PLAMIKGGERLSDLPWTVAGGDTASVRRGQVRAVVNAPE |
| | | | ARLETVGTRLRLHWDVSVAADIPFTASWSLTASDDDAVV |
| | | | QPAPALPARKRRGTPDARAAAWLEVALDDLDHLLLARRA |
| | | | TPTQPFAAAGAPWFLTLFGRDSLWTSRLLLEAADPWSLA |
| | | | LAEGTLRTLADLQGRRYDVDTAEQPGKIMHELRREANAV |
| | | | DADTVLPPLYYGTIDATELWILTLTDARRAGLAEPWVRELL |
| | | | DPLRRALSWLTTCADTDGDGFIEYVDGTGRGLANQGWK |
| | | | DSGDSIQFADGRIAQGPIALVEVQAYAHAAALAGAELLRD |
| | | | FGNGDDAVLAATATTWAAALRERFAEAFWRGDTADGYP |
| | | | VIALDARKQQVDSLASNIGHLLGTGILDRAQSRRVAELLVS |
| | | | PELNSGYGVRTLASTMAGYWPLSYHGGSVWTHDTAIAID |
| | | | GLLRDGFTSQARTLAEALLTAAPCFGFRMPELFSGTPADD |
| | | | GAPVPYPASCRPQAWAAAAGAVVARAL |
| CED91394.1 | Actinomyces | 363 | MIVNAPAQAIAAPDGSMGDAPVQGVYQSDVRALRLRALS |
| (6X-His) | succiniciruminis | | VTGSALEHLSVERCGAAASRHRALARGLDRGGADPRSIIT |
| | | | TDRTLVPADDGTVLTETLTVRIAQDRPVSTTLTLELAADLS |
| | | | PLAMIKGGERLSDLPWTVAGGDTASVRRGQVRAVVNAPE |
| | | | ARLETVGTRLRLHWDVSVAADIPFTASWSLTASDDDAVV |
| | | | QPAPALPARKRRGTPDARAAAWLEVALDDLDHLLLARRA |
| | | | TPTQPFAAAGAPWFLTLFGRDSLWTSRLLLEAADPWSLA |
| | | | LAEGTLRTLADLQGRRYDVDTAEQPGKIMHELRREANAV |
| | | | DADTVLPPLYYGTIDATELWILTLTDARRAGLAEPVVRELL |
| | | | DPLRRALSWLTTCADTDGDGFIEYVDGTGRGLANQGWK |
| | | | DSGDSIQFADGRIAQGPIALVEVQAYAHAAALAGAELLRD |
| | | | FGNGDDAVLAATATTWAAALRERFAEAFWRGDTADGYP |
| | | | VIALDARKQQVDSLASNIGHLLGTGILDRAQSRRVAELLVS |
| | | | PELNSGYGVRTLASTMAGYWPLSYHGGSVWTHDTAIAID |
| | | | GLLRDGFTSQARTLAEALLTAAPCFGFRMPELFSGTPADD |
| | | | GAPVPYPASCRPQAWAAAAGAVVARALHHHHHH |
| SLM45813.1 | Nitrospira sp. | 364 | MILEAAEEDVEEIISVNDQFYILASSSMADDRTRVLKHGET |
| | ND1 | | FGVYDRYGDIQPVGRGTQGVFHQGTRFLSRQELFLNNDR |
| | | | PMLLSSTVKEDNALLAVDLTNPDLYRDGRIAIPRGSVHVFR |
| | | | SRFLWNGVSYERFRLSNYSLAPVKMTLSIRFEADFADIFE |
| | | | VRGKKRERKGRMLPNVLRKDLLVLSYEGLDEVTREARIQF |
| | | | SPEPLEITASQATFDIELDPKGETTVAVMVACDVGDSHRP |
| | | | LLAYDAAMAEAGLAFGAEQTEDCTIQTSNAQFNEWWNRS |
| | | | VLDVRMMVTDTNEGPYPYAGVPWFSTPFGRDGVITALEC |
| | | | LWIRPELARGVLAYLASTQAKEVNPGQDAEPGKILHETRK |
| | | | GEMAALNEIPFGLYYGSVDSTPLFVMLAGAYYERTADLAFI |
| | | | QSIWANLEAALTWMDTFGDPDRDGFVEYVRKSPTGLDN |
| | | | QGWKDSHDSISHEDGSLAEGPIALCEVQGYVYDAKVQAS |
| | | | KLADALGYIDRASQLRRQARSLKERFEEAFWCEESSTYAL |
| | | | ALDGQKRPCQVKTSNAGHCLYTGIASDEHARRVAETLMS |
| | | | DELFSGWGVRTLADSERRYNPMSYHNGSIWPHDNAMIAV |
| | | | GLARYGLKSGVEKIMTGMFEVSLVLDFHRLPELFCGFVRR |
| | | | PGQGLTRYPVACNPQAWAAGSAFMALQACLGLSIIASEQ |
| | | | KVVFNYPILPEFIDKMQIKNLKVGTASVDLLLRRHDLDVGIT |
| | | | VIRRAGKVEVVSVK |
| SLM45813.1 | Nitrospira sp. | 365 | MILEAAEEDVEEIISVNDQFYILASSSMADDRTRVLKHGET |
| (6X-His) | ND1 | | FGVYDRYGDIQPVGRGTQGVFHQGTRFLSRQELFLNNDR |
| | | | PMLLSSTVKEDNALLAVDLTNPDLYRDGRIAIPRGSVHVFR |
| | | | SRFLWNGVSYERFRLSNYSLAPVKMTLSIRFEADFADIFE |
| | | | VRGKKRERKGRMLPNVLRKDLLVLSYEGLDEVTREARIQF |
| | | | SPEPLEITASQATFDIELDPKGETTVAVMVACDVGDSHRP |
| | | | LLAYDAAMAEAGLAFGAEQTEDCTIQTSNAQFNEWWNRS |
| | | | VLDVRMMVTDTNEGPYPYAGVPWFSTPFGRDGVITALEC |
| | | | LWIRPELARGVLAYLASTQAKEVNPGQDAEPGKILHETRK |
| | | | GEMAALNEIPFGLYYGSVDSTPLFVMLAGAYYERTADLAFI |
| | | | QSIWANLEAALTWMDTFGDPDRDGFVEYVRKSPTGLDN |
| | | | QGWKDSHDSISHEDGSLAEGPIALCEVQGYVYDAKVQAS |
| | | | KLADALGYIDRASQLRRQARSLKERFEEAFWCEESSTYAL |
| | | | ALDGQKRPCQVKTSNAGHCLYTGIASDEHARRVAETLMS |
| | | | DELFSGWGVRTLADSERRYNPMSYHNGSIWPHDNAMIAV |
| | | | GLARYGLKSGVEKIMTGMFEVSLVLDFHRLPELFCGFVRR |
| | | | PGQGLTRYPVACNPQAWAAGSAFMALQACLGLSIIASEQ |
| | | | KVVFNYPILPEFIDKMQIKNLKVGTASVDLLLRRHDLDVGIT |
| | | | VIRRAGKVEVVSVKHHHHHH |
| AYA22374.1 | Corallococcus sp. | 366 | MQVKQVLRETGVVLAAGLLYGSAAFAQSSVIIGTVINTEDK |
| | EGB | | KPAADVVVTATSPNLQGEQTVVTDAQGNYRIPQLPPGTY |
| | | | TLRFEKETFKPYARPEIQLLLNRTIRVNVELLPDSFTQTVD |
| | | | VIGTPPTIDVGSTNQGVNVDQEFIKRIAVARPVGKGGATR |
| | | | SFESLAELAPGAQADQYGVSINGTTSPENGYVVDGLSTN |
| | | | DPAFGVNASPLSIEFVQDVNIITGGYMPEFGRSTGGVINAV |
| | | | TRSGSNEFHGSVFANWTPGALEGKRKLVVEDGSTITGLN |
| | | | ALSNLGDFGATLGGPILKDKLWFFAGFAPSFQRYQHTRAL |
| | | | NAFQLTAPFDPTPGATNTVAKDANGSSVVQEIPGSERKYF |
| | | | ADARTIQFMGKLTYLINQDHNVSFALNGTPTVSGGLGKLRI |
| | | | NPKTGGLPGAQISRPEDIGQVEDRANTTALALKYAGAFMD |
| | | | KKVLVDANVGWFHQTAGVTPADGTALGSTDGLAGYSLAQ |
| | | | YTRRRTLTTFESVPNAAEYCGATTAEQLLRCPSTNYLVGG |
| | | | PGFMSEATLDRYQINAKATYLLNALGTHVFKAGVDTEFLN |
| | | | FDQKKAYSGSVYLVEAGSASDQMYQGVSRHIWSDNRRY |
| | | | GYQTAPDTPVFETLQQSSTSGTTIGGFLQDSWSIANRVTL |
| | | | NLGVRYDVQNMYGGDGNLALKLGNQWSPRLGAVVDPFA |
| | | | NGRAKLFVNFARYYEQVPLNMMDRAFPGERRFSARRYLT |
| | | | EPGGAEAGCDPSTLEGQRGGCSDRQYIVPRAESTLNPN |
| | | | QYFSGGLVQSEPVDPDIKPQSSDELVVGGEYELLANTRV |
| | | | GASYTHRDMNSVIEDMSRDDGNTYFLGNPGSGFAKEFPK |
| | | | PVRDYDAVTVYLNRTFTDGWLAQASYTWSRLYGNYPGLF |
| | | | RPENNQLDPNILADFDLIALLDNRTGLLPFDRTHAIKVFGA |
| | | | KEFNISNALSASIGLSYRGNSGTPISYLGAYPGYGQDETFI |
| | | | LPRGSGGRTPWVNSVDSNVGVNYRVSKDSVVSFTLDVF |
| | | | NLFNFQTADRVDQSYTFSEVLPVYDPKTGKAGTAADLPTK |
| | | | TSPGSVPSAEGGVLAYDDVNQNFKNPDRYQAPRQIRFGV |
| | | | RYTF |
| AYA22374.1 | Corallococcus sp. | 367 | QSSVIIGTVINTEDKKPAADVVVTATSPNLQGEQTVVTDAQ |
| (6X-His) | EGB | | GNYRIPQLPPGTYTLRFEKETFKPYARPEIQLLLNRTIRVN |
| | | | VELLPDSFTQTVDVIGTPPTIDVGSTNQGVNVDQEFIKRIA |
| | | | VARPVGKGGATRSFESLAELAPGAQADQYGVSINGTTSP |
| | | | ENGYVVDGLSTNDPAFGVNASPLSIEFVQDVNIITGGYMP |
| | | | EFGRSTGGVINAVTRSGSNEFHGSVFANWTPGALEGKRK |
| | | | LVVEDGSTITGLNALSNLGDFGATLGGPILKDKLWFFAGFA |
| | | | PSFQRYQHTRALNAFQLTAPFDPTPGATNTVAKDANGSS |
| | | | WVQEIPGSERKYFADARTIQFMGKLTYLINQDHNVSFALN |
| | | | GTPTVSGGLGKLRINPKTGGLPGAQISRPEDIGQVEDRAN |
| | | | TTALALKYAGAFMDKKVLVDANVGWFHQTAGVTPADGTA |
| | | | LGSTDGLAGYSLAQYTRRRTLTTFESVPNAAEYCGATTAE |
| | | | QLLRCPSTNYLVGGPGFMSEATLDRYQINAKATYLLNALG |
| | | | THVFKAGVDTEFLNFDQKKAYSGSVYLVEAGSASDQMYQ |
| | | | GVSRHIWSDNRRYGYQTAPDTPVFETLQQSSTSGTTIGG |
| | | | FLQDSWSIANRVTLNLGVRYDVQNMYGGDGNLALKLGNQ |
| | | | WSPRLGAVVDPFANGRAKLFVNFARYYEQVPLNMMDRA |
| | | | FPGERRFSARRYLTEPGGAEAGCDPSTLEGQRGGCSDR |
| | | | QYIVPRAESTLNPNQYFSGGLVQSEPVDPDIKPQSSDELV |
| | | | VGGEYELLANTRVGASYTHRDMNSVIEDMSRDDGNTYFL |
| | | | GNPGSGFAKEFPKPVRDYDAVTVYLNRTFTDGWLAQASY |
| | | | TWSRLYGNYPGLFRPENNQLDPNILADFDLIALLDNRTGLL |
| | | | PFDRTHAIKVFGAKEFNISNALSASIGLSYRGNSGTPISYL |
| | | | GAYPGYGQDETFILPRGSGGRTPWVNSVDSNVGVNYRV |
| | | | SKDSVVSFTLDVFNLFNFQTADRVDQSYTFSEVLPVYDPK |
| | | | TGKAGTAADLPTKTSPGSVPSAEGGVLAYDDVNQNFKNP |
| | | | DRYQAPRQIRFGVRYTFHHHHHH |
| VXD17547.1 | Planktothrix | 368 | MSINFGREICGDLTSAETREWLVTNGIGGYASGTIAGLLTR |
| | paucivesiculata | | RYHGLLVAALNPPLGRTLLVTKLDEIVEYHEQVYPLYTNR |
| | PCC 9631 | | WSGGAINGYGHHHIEKFHLDRTIPTWKFACADTLLEKRIW |
| | | | MQPEANITYVRYSLTRASGPITLTLKAFVNYRDYHSDTHA |
| | | | WNWKMQAEAVENGLCITAFPQASPIYLLTDRGTVQPTHQ |
| | | | WCYNFDLAREHDRGLMDHEDHLHIATFSATLHPGESFCF |
| | | | VASTEPNPGLNAEAALELRRTYERKLLGYWQDSCPSNIRE |
| | | | VPEWVQQLVFAADQFVVNRAIPENPIGKTIIAGYPWFGDW |
| | | | GRDTMISLPGITMTTGRLQITRAILYTYSKCVEEGMLPNRF |
| | | | TDTGETPDYNTVDATLWYFEAIRQYYDLTEDIDIVENLFPV |
| | | | LADIIQYHCRGTRYNIHLDPADGLLYAGEQGSQLTWMDAK |
| | | | IGDWVVTPRIGKPVEINALWYNALRIMGKFARQIGRPYQE |
| | | | YDALADRALARFERFWNPDLGYCYDVIDGIYGDDASLRPN |
| | | | QIFAVSLPHSALSPDQQRAVVETCGRYLLTSSGLRSLAPN |
| | | | HFHYRGYYEGNPVQRDSAYHQGTVWAWLLGPFVIAHLR |
| | | | VFQNPTVARSFLEPISNHLTTGGLGTISEIFDGNAPMYPPG |
| | | | LYCPSLVSGRSSAGLVSHRTLDRGSWKIMGLKAVLFDFN |
| | | | GIIINDESIHEQLIAELLLSENLRSKPEEIRKFSLGRSDRACL |
| | | | TDLLTYRGRVVSNTYLNQLIERKSISYRNYLEQLEQLPLYL |
| | | | GLENFLQQLQLADLKLGIVTGALRAEVELVLTRSGLKDYFP |
| | | | VIVAGDDITLSKPEPEGYLLGLQRLNEAYPYLNLQPSECLA |
| | | | VEDTCVGIIAAGRAGMQVAGVAHTYPLHILQRWANWAVD |
| | | | RFSDLELERIQKNFLEEPLQSQV |
| VXD17547.1 | Planktothrix | 369 | MSINFGREICGDLTSAETREWLVTNGIGGYASGTIAGLLTR |
| (6X-His) | paucivesiculata | | RYHGLLVAALNPPLGRTLLVTKLDEIVEYHEQVYPLYTNR |
| | PCC 9631 | | WSGGAINGYGHHHIEKFHLDRTIPTWKFACADTLLEKRIW |
| | | | MQPEANITYVRYSLTRASGPITLTLKAFVNYRDYHSDTHA |
| | | | WNWKMQAEAVENGLCITAFPQASPIYLLTDRGTVQPTHQ |
| | | | WCYNFDLAREHDRGLMDHEDHLHIATFSATLHPGESFCF |
| | | | VASTEPNPGLNAEAALELRRTYERKLLGYWQDSCPSNIRE |
| | | | VPEWVQQLVFAADQFVVNRAIPENPIGKTIIAGYPWFGDW |
| | | | GRDTMISLPGITMTTGRLQITRAILYTYSKCVEEGMLPNRF |
| | | | TDTGETPDYNTVDATLWYFEAIRQYYDLTEDIDIVENLFPV |
| | | | LADIIQYHCRGTRYNIHLDPADGLLYAGEQGSQLTWMDAK |
| | | | IGDWVVTPRIGKPVEINALWYNALRIMGKFARQIGRPYQE |
| | | | YDALADRALARFERFWNPDLGYCYDVIDGIYGDDASLRPN |
| | | | QIFAVSLPHSALSPDQQRAVVETCGRYLLTSSGLRSLAPN |
| | | | HFHYRGYYEGNPVQRDSAYHQGTVWAWLLGPFVIAHLR |
| | | | VFQNPTVARSFLEPISNHLTTGGLGTISEIFDGNAPMYPPG |
| | | | LYCPSLVSGRSSAGLVSHRTLDRGSWKIMGLKAVLFDFN |
| | | | GIIINDESIHEQLIAELLLSENLRSKPEEIRKFSLGRSDRACL |
| | | | TDLLTYRGRVVSNTYLNQLIERKSISYRNYLEQLEQLPLYL |
| | | | GLENFLQQLQLADLKLGIVTGALRAEVELVLTRSGLKDYFP |
| | | | VIVAGDDITLSKPEPEGYLLGLQRLNEAYPYLNLQPSECLA |
| | | | VEDTCVGIIAAGRAGMQVAGVAHTYPLHILQRWANWAVD |
| | | | RFSDLELERIQKNFLEEPLQSQVHHHHHH |
| ABN93721.1 | Burkholderia | 370 | MPRQADPASPRAPQAAAPASSSARCAAPGAAPPQSAQP |
| | pseudomallei | | PQSAQPPQSAQPPQSAQPPQSAQPPQSPQSPQPAPSTP |
| | (strain 1106a) | | AFIAPEPDAQKAVRGSQFVLKAGDAFVVSDALGDIAGRDD |
| | | | GLFVDDMRVLSQWRLTFGGRAPSLLSGATSADNASFTAH |
| | | | LTNRPLPPLGGRETPEGVIHIERMRVLADNVLHEALTLTNY |
| | | | GTCDAEVPLSVSFGADFKDMFEVRGSRRERRGAVAPPC |
| | | | VEAGAVRLRYDGLDAVERSVRIGFEPKPDTLAVDRADYTL |
| | | | TIAAQACVSLYLTVEARVGAAHAGGDAFARRPCVATGRG |
| | | | ALRKALADVHRAMRERRRTIARVHTSNPLFNAWLDRSLA |
| | | | DLGLLTTSLETGPYPYAGIPWFSTPFGRDAVITSLQMLWL |
| | | | QPSLARGVLRFLAEHQARETSAFRDAEPGKIMHEFRKSE |
| | | | MAATGEVPFALYYGGVDTTPLFVVLAGAYLEHTGDESLID |
| | | | ELWPALERAAQWVSTVCERNPRGLLDYQRTSARGLANQ |
| | | | GWKDSQDSVFHADGRFPSGPIALVEVQAYACAAFDAMAT |
| | | | FSRRRGHAADTVRYAQRAKRLREQVEALFWMPEAGFYGI |
| | | | AVDGHGELCRVLASNAAHLLAFGLPEQSRGESVARVLGS |
| | | | TLFRTGWGVRTLAAGQPRFNPMAYHNGSIWPHDNALAA |
| | | | RGLARYGDKRAVLDLLRALFEAAVSFDMRLPELFCGFPR |
| | | | RRGEPPTAYPVACLPQAWAAGAPFMMLQACLGISVDAAR |
| | | | GEVRVERPELPEGVDWLRVDDLRVGGDSVSLTFRRVEG |
| | | | QVVAAAEPGGARVVAVL |
| ABN93721.1 | Burkholderia | 371 | MPRQADPASPRAPQAAAPASSSARCAAPGAAPPQSAQP |
| (6X-His) | pseudomallei | | PQSAQPPQSAQPPQSAQPPQSAQPPQSPQSPQPAPSTP |
| | (strain 1106a) | | AFIAPEPDAQKAVRGSQFVLKAGDAFVVSDALGDIAGRDD |
| | | | GLFVDDMRVLSQWRLTFGGRAPSLLSGATSADNASFTAH |
| | | | LTNRPLPPLGGRETPEGVIHIERMRVLADNVLHEALTLTNY |
| | | | GTCDAEVPLSVSFGADFKDMFEVRGSRRERRGAVAPPC |
| | | | VEAGAVRLRYDGLDAVERSVRIGFEPKPDTLAVDRADYTL |
| | | | TIAAQACVSLYLTVEARVGAAHAGGDAFARRPCVATGRG |
| | | | ALRKALADVHRAMRERRRTIARVHTSNPLFNAWLDRSLA |
| | | | DLGLLTTSLETGPYPYAGIPWFSTPFGRDAVITSLQMLWL |
| | | | QPSLARGVLRFLAEHQARETSAFRDAEPGKIMHEFRKSE |
| | | | MAATGEVPFALYYGGVDTTPLFVVLAGAYLEHTGDESLID |
| | | | ELWPALERAAQWVSTVCERNPRGLLDYQRTSARGLANQ |
| | | | GWKDSQDSVFHADGRFPSGPIALVEVQAYACAAFDAMAT |
| | | | FSRRRGHAADTVRYAQRAKRLREQVEALFWMPEAGFYGI |
| | | | AVDGHGELCRVLASNAAHLLAFGLPEQSRGESVARVLGS |
| | | | TLFRTGWGVRTLAAGQPRFNPMAYHNGSIWPHDNALAA |
| | | | RGLARYGDKRAVLDLLRALFEAAVSFDMRLPELFCGFPR |
| | | | RRGEPPTAYPVACLPQAWAAGAPFMMLQACLGISVDAAR |
| | | | GEVRVERPELPEGVDWLRVDDLRVGGDSVSLTFRRVEG |
| | | | QVVAAAEPGGARVVAVLHHHHHH |
| ACC84370.1 | Nostoc | 372 | MPDLDTREWLLTNGLGSFASGTISDVRTRTYHGWLFTAT |
| | punctiforme | | NPPSGRTLLFSHLDASLEVSGNVVALGTNVWGNGQIELT |
| | (strain ATCC | | GYELLRCFDINPVPKWIWGRDNWQLSRQLVMPYGLVEAG |
| | 29133/PCC | | DTSTSSVHRWRLGIRNEELEIAKNASQCPMTAVAPLGETP |
| | 73102) | | SRSVSQRRPHCLPNAQFCHRILIQYRYDGNDTAILRLRLLI |
| | | | AERDFHHPQIASSGLQFSELLGQQQICLQAKKLGNFGIPW |
| | | | HLRWTKGKYQPDAVWYWNYGLSEETKRGLSDKEDLYSP |
| | | | GYLIVRLQPGDVVTLEARVGFPNLMADVLTSETFAEAVEV |
| | | | EQDRLSQIFGWSEESREQGAREKSFPLYPPHPAPVPLLN |
| | | | AQSLLWQQLLKASDQFIVYRASIAGPTMIAGYHWFNERGR |
| | | | DILIALPGLALVPQRFDLAKGVLRTFRHYYRHGLMPNVFPD |
| | | | ADGEPLYNSIDAALWWVETLGLYLEATQDWEFLAEQFSV |
| | | | VQQIHKAFVGGTHYNIQVDAIDGLISWDARGVALTWMDVV |
| | | | IGANPVTPRHGKPVEINALWYSALCWLSRWAERLSSLELG |
| | | | DPVRLTKQAQRYALQAEKVKTSLQKFWNPQLGYLYDTIEP |
| | | | DDRRNFQIRPNAVLALSLHHCGFSAQQGSQILDLATISLLT |
| | | | PYGLRSLDPGDPEYKGRYEGNQEQRDRAYHQGTVWGW |
| | | | LIGPYIRAWQRFYPEQSLPFDWQPLLDHFLSGACLGSISEI |
| | | | FDGDAPHTPRGAIAQAWSVAEIIRHIQK |
| ACC84370.1 | Nostoc | 373 | MPDLDTREWLLTNGLGSFASGTISDVRTRTYHGWLFTAT |
| (6X-His) | punctiforme | | NPPSGRTLLFSHLDASLEVSGNVVALGTNVWGNGQIELT |
| | (strain ATCC | | GYELLRCFDINPVPKWIWGRDNWQLSRQLVMPYGLVEAG |
| | 29133/PCC | | DTSTSSVHRWRLGIRNEELEIAKNASQCPMTAVAPLGETP |
| | 73102) | | SRSVSQRRPHCLPNAQFCHRILIQYRYDGNDTAILRLRLLI |
| | | | AERDFHHPQIASSGLQFSELLGQQQICLQAKKLGNFGIPW |
| | | | HLRWTKGKYQPDAVWYWNYGLSEETKRGLSDKEDLYSP |
| | | | GYLIVRLQPGDVVTLEARVGFPNLMADVLTSETFAEAVEV |
| | | | EQDRLSQIFGWSEESREQGAREKSFPLYPPHPAPVPLLN |
| | | | AQSLLWQQLLKASDQFIVYRASIAGPTMIAGYHWFNERGR |
| | | | DILIALPGLALVPQRFDLAKGVLRTFRHYYRHGLMPNVFPD |
| | | | ADGEPLYNSIDAALWWWVETLGLYLEATQDWEFLAEQFSV |
| | | | VQQIHKAFVGGTHYNIQVDAIDGLISWDARGVALTWMDVV |
| | | | IGANPVTPRHGKPVEINALWYSALCWLSRWAERLSSLELG |
| | | | DPVRLTKQAQRYALQAEKVKTSLQKFWNPQLGYLYDTIEP |
| | | | DDRRNFQIRPNAVLALSLHHCGFSAQQGSQILDLATISLLT |
| | | | PYGLRSLDPGDPEYKGRYEGNQEQRDRAYHQGTVWGW |
| | | | LIGPYIRAWQRFYPEQSLPFDWQPLLDHFLSGACLGSISEI |
| | | | FDGDAPHTPRGAIAQAWSVAEIIRHIQKHHHHHH |
| ACM06640.1 | Thermomicrobium | 374 | MGLARDARILKDGELFVVSDERGDLRRTLPGAGVYARDT |
| | roseum (strain | | RFLSELWLLLNGEEPELFDSSAERTNTAIFEFGNAVFRDE |
| | ATCC 27502/ | | EGRDILAHTISLRRCRSVRRGTLDEELVLENYNAFDITVEL |
| | DSM 5159/P-2) | | AFVFAADFLDIFEVRGFPRERPRGRILLPQQQDHSLRLAY |
| | | | RAPDDVLLETEIVFSRQPDTVTTEPGATHLSELALPRMLLP |
| | | | GHDQLIRAPGMIHPPRAFVLFHLKLPSHGRERLALRIAPRE |
| | | | LLERRIAVRVPDVSRLTEPVEPPTYSFARARTDHELFDRVL |
| | | | QRSLDDVRALLTPFPGGRLVAAGIPWFVAPFGRDSLIVSL |
| | | | QMMLFAPELALETLRFLARYQGRKHDPWTEEEPGKILHEL |
| | | | RFGEMVRLGETPHSPYYGTVDATPLFIVTFCEVMDWVGT |
| | | | PALFDELFPAVQAALHWIDQHGDHNGDGFIDYGRVSPRG |
| | | | LVHQNWKDSHNSLLFPDGRDPVPPIAAVEVQGYVYQAKR |
| | | | ALAAVLQRFGKPSHQVLVARLRAEAEQLRARFEQRFWSE |
| | | | SDQFYGQAIDGQGRLVPAISSNPGHCLWSEIIGPERAARV |
| | | | VERLFRSDLYSGWGIRTLASTMPHYNPMSYHNGSIWPHD |
| | | | NALIIAGLRRYGYHREALRLASDLLEVAVSFPYYRLPELFC |
| | | | GFGRDEAECMVPISYPVSCSPQAWAAGTMPFLLRIMLGA |
| | | | EARAAERRLTLRPAFPDWLTEVEIRGLRFAGTTLDLAVRR |
| | | | QAGRYTLEVEADPGITVVLAGSSDDFRR |
| ACM06640.1 | Thermomicrobium | 375 | MGLARDARILKDGELFVVSDERGDLRRTLPGAGVYARDT |
| (6X-His) | roseum (strain | | RFLSELWLLLNGEEPELFDSSAERTNTAIFEFGNAVFRDE |
| | ATCC 27502/ | | EGRDILAHTISLRRCRSVRRGTLDEELVLENYNAFDITVEL |
| | DSM 5159/P-2) | | AFVFAADFLDIFEVRGFPRERPRGRILLPQQQDHSLRLAY |
| | | | RAPDDVLLETEIVFSRQPDTVTTEPGATHLSELALPRMLLP |
| | | | GHDQLIRAPGMIHPPRAFVLFHLKLPSHGRERLALRIAPRE |
| | | | LLERRIAVRVPDVSRLTEPVEPPTYSFARARTDHELFDRVL |
| | | | QRSLDDVRALLTPFPGGRLVAAGIPWFVAPFGRDSLIVSL |
| | | | QMMLFAPELALETLRFLARYQGRKHDPWTEEEPGKILHEL |
| | | | RFGEMVRLGETPHSPYYGTVDATPLFIVTFCEVMDWVGT |
| | | | PALFDELFPAVQAALHWIDQHGDHNGDGFIDYGRVSPRG |
| | | | LVHQNWKDSHNSLLFPDGRDPVPPIAAVEVQGYVYQAKR |
| | | | ALAAVLQRFGKPSHQVLVARLRAEAEQLRARFEQRFWSE |
| | | | SDQFYGQAIDGQGRLVPAISSNPGHCLWSEIIGPERAARV |
| | | | VERLFRSDLYSGWGIRTLASTMPHYNPMSYHNGSIWPHD |
| | | | NALIIAGLRRYGYHREALRLASDLLEVAVSFPYYRLPELFC |
| | | | GFGRDEAECMVPISYPVSCSPQAWAAGTMPFLLRIMLGA |
| | | | EARAAERRLTLRPAFPDWLTEVEIRGLRFAGTTLDLAVRR |
| | | | QAGRYTLEVEADPGITVVLAGSSDDFRRHHHHHH |
| ACN15454.1 | Desulforapulum | 376 | MNVSEMISQLPRPGTSRVMFCGDVLTFTLSIPSNVSGMA |
| | autotrophicum | | WIRTNLGRASIARNEIIDEVDRNEIKLDEAWHDIQMTRQGP |
| | (strain ATCC | | AVFAITLPLSETGHFRAKCFFWAEDSHAPVWPPGEDTIINV |
| | 43914/DSM | | EPAGTCCANIVYNAFVRQFGPTRSQGQPLAQEEADCIQC |
| | 3382/VKM B- | | LDKRGYAVIPSSGKFRDLSGEIDFILTHLGCRAIHLLPIHPT |
| | 1955/HRM2) | | PTTYGRMGRFGSPYAALNFTDVDPGLAEFDSSATPMEQF |
| | | | SALVDKIHSCAGYLILDIAINHTGWAASIHEKHPEWLVRNK |
| | | | EGEIAVPGAWGVLWADLTRLDYSKKALWQYMADIFLLWC |
| | | | TRGVDGFRCDAGYMIPVPAWEYIVAKVKKEYPDTLFFLEG |
| | | | LGGPLDATCDILSQANFNWAYSELFQNYDRKAVEAYLPTA |
| | | | ISISEKFGHMIHFAETHDNQRLAATSTRYAKMRTSLCALFS |
| | | | VCGGFGFANGVEWFATEKINVHESTCLNWGAKINQTDHI |
| | | | RRLNLILKTHPAFADNVSLDLVQTGQGNYLVLCRRIAATDR |
| | | | EIIVVANLDCKMSQTVLWQARHQPEGVLFDLVTEQQVVAK |
| | | | LEGEILSVEVEPGRVMALTRDRQDIKRLQAADLKNDPIPW |
| | | | RWVRQKQVACALKIVTTLAGYGDVSGLDLDYEADALVADP |
| | | | VAFIRSFNPDGNESRVVVWEWNRDVRRQVMVPHGFFLM |
| | | | WVAPANFRAEIRTRTQGTVRTLEQNEALATRDGRFFAVFL |
| | | | PFDTQGRVMEHTLKIRVLGAKGTQKAKAPLVFLPGSGTAF |
| | | | VSSTHTRKAIAKNPSLKLLGVNHRGAMMRAFAWWGRLES |
| | | | RYDALLAANIQPDFPEERWMALTRFRIWATFQGYSRELTL |
| | | | DCLESFTFSYGNRGLWRFRLPTSEGSHCPIEIALKMVDQT |
| | | | NAIEMDITRPAGSPRRNPLGDDKQVTIIIRPDLEHRSFHET |
| | | | VKAWKGPENQWKSSISMVSGGFLFAPDPNHCTLGVKAS |
| | | | DGTFVWEPEWQYMVHRSLDAERGLDPDSDLFSPGYFIAN |
| | | | LGGRDTIRISARVTCGKGPERDDILTGSDGPDTEFVRGWP |
| | | | LPEALTKSLDAFLVDRAKDRSVIAGYPWFLDWGRDSLIFS |
| | | | RALIQGERLVDAKKILRLFGRFEANGSLPNMISGNDARNR |
| | | | ETSDAPLWFIAAAREVVEKEGGALLDQTLGGRTFKEILVSI |
| | | | GDAYVRGTQTGIAMDPETALVFSPSHFTWMDTNFPAGTP |
| | | | REGYPIEIQALWAYSLEFLAKIDPNGTQAWARLADQVKDSI |
| | | | KTLFFMESRGYFSDCLHTRGKAPAGSAVVDDALRPNQLF |
| | | | LISLGVIRDKDLCIPALEHCMELLVPGAIRTLDDSPVDLPLVI |
| | | | EGPTGPLNDPHRPYWGHYMGDEDTRRKPAYHNGTAWT |
| | | | WVFPVFCEAWAETFGSKGAMTGISWLTSSVKLMEQGVA |
| | | | GFVPEIVDGDAPHAPRGCDAQAWGASELYRVWHKLSLR |
| | | | S |
| ACN15454.1 | Desulforapulum | 377 | MNVSEMISQLPRPGTSRVMFCGDVLTFTLSIPSNVSGMA |
| (6X-His) | autotrophicum | | WIRTNLGRASIARNEIIDEVDRNEIKLDEAWHDIQMTRQGP |
| | (strain ATCC | | AVFAITLPLSETGHFRAKCFFWAEDSHAPVWPPGEDTIINV |
| | 43914/DSM | | EPAGTCCANIVYNAFVRQFGPTRSQGQPLAQEEADCIQC |
| | 3382/VKM B- | | LDKRGYAVIPSSGKFRDLSGEIDFILTHLGCRAIHLLPIHPT |
| | 1955/HRM2) | | PTTYGRMGRFGSPYAALNFTDVDPGLAEFDSSATPMEQF |
| | | | SALVDKIHSCAGYLILDIAINHTGWAASIHEKHPEWLVRNK |
| | | | EGEIAVPGAWGVLWADLTRLDYSKKALWQYMADIFLLWC |
| | | | TRGVDGFRCDAGYMIPVPAWEYIVAKVKKEYPDTLFFLEG |
| | | | LGGPLDATCDILSQANFNWAYSELFQNYDRKAVEAYLPTA |
| | | | ISISEKFGHMIHFAETHDNQRLAATSTRYAKMRTSLCALFS |
| | | | VCGGFGFANGVEWFATEKINVHESTCLNWGAKINQTDHI |
| | | | RRLNLILKTHPAFADNVSLDLVQTGQGNYLVLCRRIAATDR |
| | | | EIIVVANLDCKMSQTVLWQARHQPEGVLFDLVTEQQVVAK |
| | | | LEGEILSVEVEPGRVMALTRDRQDIKRLQAADLKNDPIPW |
| | | | RWVRQKQVACALKIVTTLAGYGDVSGLDLDYEADALVADP |
| | | | VAFIRSFNPDGNESRVVVWEWNRDVRRQVMVPHGFFLM |
| | | | WVAPANFRAEIRTRTQGTVRTLEQNEALATRDGRFFAVFL |
| | | | PFDTQGRVMEHTLKIRVLGAKGTQKAKAPLVFLPGSGTAF |
| | | | VSSTHTRKAIAKNPSLKLLGVNHRGAMMRAFAWWGRLES |
| | | | RYDALLAANIQPDFPEERWMALTRFRIWATFQGYSRELTL |
| | | | DCLESFTFSYGNRGLWRFRLPTSEGSHCPIEIALKMVDQT |
| | | | NAIEMDITRPAGSPRRNPLGDDKQVTIIIRPDLEHRSFHET |
| | | | VKAWKGPENQWKSSISMVSGGFLFAPDPNHCTLGVKAS |
| | | | DGTFVWEPEWQYMVHRSLDAERGLDPDSDLFSPGYFIAN |
| | | | LGGRDTIRISARVTCGKGPERDDILTGSDGPDTEFVRGWP |
| | | | LPEALTKSLDAFLVDRAKDRSVIAGYPWFLDWGRDSLIFS |
| | | | RALIQGERLVDAKKILRLFGRFEANGSLPNMISGNDARNR |
| | | | ETSDAPLWFIAAAREVVEKEGGALLDQTLGGRTFKEILVSI |
| | | | GDAYVRGTQTGIAMDPETALVFSPSHFTWMDTNFPAGTP |
| | | | REGYPIEIQALWAYSLEFLAKIDPNGTQAWARLADQVKDSI |
| | | | KTLFFMESRGYFSDCLHTRGKAPAGSAVVDDALRPNQLF |
| | | | LISLGVIRDKDLCIPALEHCMELLVPGAIRTLDDSPVDLPLVI |
| | | | EGPTGPLNDPHRPYWGHYMGDEDTRRKPAYHNGTAWT |
| | | | WVFPVFCEAWAETFGSKGAMTGISWLTSSVKLMEQGVA |
| | | | GFVPEIVDGDAPHAPRGCDAQAWGASELYRVWHKLSLR |
| | | | SHHHHHH |
| ADE70946.1 | Priestia | 378 | MDYRVIKENDLFLLTDPAGNINENHQYGLGLYMKDTRFLS |
| | megaterium | | KFRLKINDADPVLLSSSAEENLLATILLTNPHMEKEGELVL |
| | (strain ATCC | | WRESIEIERKRFIYGGVFYEKIKLKSYFPKKIAFTLSIQADVD |
| | 12872/ | | FNDMFIVRGFQTGNVGKRTGEIKGESSLTYTYKGADDIKR |
| | QMB1551) | | ATRILWDKEASYVDEQGEVTFSFELNHQEEEEITLMVQPQ |
| | | | MGEDINENILSADEALSLLTQSYKKWEKSSTKVTTDHPTM |
| | | | QRLITRGLNDLRVLLTDVGFGAFPVAGLPWFGVPFGRDSL |
| | | | IAALQMLAFNPEVAKGTLRTMAAYQGTANDPWRDEQPGK |
| | | | IMHEIRYGELANTNQIPFTPYYGTVDATPLFLILLTEYVKWT |
| | | | GDLEFVQEMHMTIDRALTWINDYGDRDGDLFLEYHQESS |
| | | | KGIANQGWKDSGDSVVHRTGEYAKTPIALVEVQGYVYQA |
| | | | KMGIASLYDQLNKTDEANNLRKEARQLRKKFNEAFWMDD |
| | | | VSFYAIALDEHKSQVGTITSNPGHALLAEIMDEEKLHKVSE |
| | | | RLISDSMFSGYGIRTMAEGEAGYNPMSYHDGSIWPHDNS |
| | | | LIVLGLSKANKQTEANKVIKGLVDAAEHFEYDRLPELFCGY |
| | | | SSKLDKPVKYPVACSPQAWAAGTPLAFVQAMLRLFPDST |
| | | | AGTIQINPVLIDDMNELSVENLVIGSGKLSLTVKKEESETIIY |
| | | | VKENTTKYELLSQCKVRNYVG |
| ADE70946.1 | Priestia | 379 | MDYRVIKENDLFLLTDPAGNINENHQYGLGLYMKDTRFLS |
| (6X-His) | megaterium | | KFRLKINDADPVLLSSSAEENLLATILLTNPHMEKEGELVL |
| | (strain ATCC | | WRESIEIERKRFIYGGVFYEKIKLKSYFPKKIAFTLSIQADVD |
| | 12872/ | | FNDMFIVRGFQTGNVGKRTGEIKGESSLTYTYKGADDIKR |
| | QMB1551) | | ATRILWDKEASYVDEQGEVTFSFELNHQEEEEITLMVQPQ |
| | | | MGEDINENILSADEALSLLTQSYKKWEKSSTKVTTDHPTM |
| | | | QRLITRGLNDLRVLLTDVGFGAFPVAGLPWFGVPFGRDSL |
| | | | IAALQMLAFNPEVAKGTLRTMAAYQGTANDPWRDEQPGK |
| | | | IMHEIRYGELANTNQIPFTPYYGTVDATPLFLILLTEYVKWT |
| | | | GDLEFVQEMHMTIDRALTWINDYGDRDGDLFLEYHQESS |
| | | | KGIANQGWKDSGDSVVHRTGEYAKTPIALVEVQGYVYQA |
| | | | KMGIASLYDQLNKTDEANNLRKEARQLRKKFNEAFWMDD |
| | | | VSFYAIALDEHKSQVGTITSNPGHALLAEIMDEEKLHKVSE |
| | | | RLISDSMFSGYGIRTMAEGEAGYNPMSYHDGSIWPHDNS |
| | | | LIVLGLSKANKQTEANKVIKGLVDAAEHFEYDRLPELFCGY |
| | | | SSKLDKPVKYPVACSPQAWAAGTPLAFVQAMLRLFPDST |
| | | | AGTIQINPVLIDDMNELSVENLVIGSGKLSLTVKKEESETIIY |
| | | | VKENTTKYELLSQCKVRNYVGHHHHHH |
| CCC98981.1 | Azospirillum | 380 | MADFIRKITRDALRSAPDAALSREWLVANGLGGYASSSLS |
| | baldaniorum | | GAVTRRYHGLLVAALPAPLGRVVMLSQLNDVLIERDGTER |
| | | | RLTGHNPQADQWPDPNYPSDVAPSGLAEFRMESGLPVW |
| | | | RYEIGDVVIEKQIVLPHLQNIVHVTYRVVKAPQPICLRLRPV |
| | | | MAFRKLESAVDEPLARDYRVTARGHEYEIWAGPELPVLR |
| | | | MTIEGAELPLTLDGGARRDIVYPVEAERGYESRGSLWTPG |
| | | | YFSGPLSQGQTLSFVAATEPWQRLWALPPADVLRFEKER |
| | | | RRRIVGTAHPSLREGAMAELVLSADSFLFVPAGRVADQM |
| | | | RARAEGDEVRTVIAGYHWFTDWGRDTMISLEGLTLTTGR |
| | | | HMEAGWILRTFAHYIRDGLIPNMFPDGKDEGLYHTADATL |
| | | | WFFHALNRYVQATGDRHTLQLLLPRLVEVIDHHRRGTRF |
| | | | GIAVDPGDGLLRQGREGYQLTWMDAKVEDWVVTPRRGK |
| | | | AVEINALWYNALCLTAGWLTEEGDAQAARPLAELAEQAR |
| | | | VSFNARFWCAHAGHLYDVVDGERGDDDACRPNQIFAVSL |
| | | | DNPVLDRSRWEPVVETVRQRLLTPVGLRSLAPGQRDYKP |
| | | | KYFGDLRARDAAYHQGTVWGWLIGPYVDAWLKLHPEEK |
| | | | AGARELLQGFLPHLDQAGIGTISEIFDAEPPFSPRGCISQA |
| | | | WSVAEVLRCWAATAEDR |
| CCC98981.1 | Azospirillum | 38 | MADFIRKITRDALRSAPDAALSREWLVANGLGGYASSSLS |
| (6X-His) | baldaniorum | | GAVTRRYHGLLVAALPAPLGRVVMLSQLNDVLIERDGTER |
| | | | RLTGHNPQADQWPDPNYPSDVAPSGLAEFRMESGLPVW |
| | | | RYEIGDVVIEKQIVLPHLQNIVHVTYRVVKAPQPICLRLRPV |
| | | | MAFRKLESAVDEPLARDYRVTARGHEYEIWAGPELPVLR |
| | | | MTIEGAELPLTLDGGARRDIVYPVEAERGYESRGSLWTPG |
| | | | YFSGPLSQGQTLSFVAATEPWQRLWALPPADVLRFEKER |
| | | | RRRIVGTAHPSLREGAMAELVLSADSFLFVPAGRVADQM |
| | | | RARAEGDEVRTVIAGYHWFTDWGRDTMISLEGLTLTTGR |
| | | | HMEAGWILRTFAHYIRDGLIPNMFPDGKDEGLYHTADATL |
| | | | WFFHALNRYVQATGDRHTLQLLLPRLVEVIDHHRRGTRF |
| | | | GIAVDPGDGLLRQGREGYQLTWMDAKVEDWVVTPRRGK |
| | | | AVEINALWYNALCLTAGWLTEEGDAQAARPLAELAEQAR |
| | | | VSFNARFWCAHAGHLYDVVDGERGDDDACRPNQIFAVSL |
| | | | DNPVLDRSRWEPVVETVRQRLLTPVGLRSLAPGQRDYKP |
| | | | KYFGDLRARDAAYHQGTVWGWLIGPYVDAWLKLHPEEK |
| | | | AGARELLQGFLPHLDQAGIGTISEIFDAEPPFSPRGCISQA |
| | | | WSVAEVLRCWAATAEDRHHHHHH |
| CCF83107.1 | Nitrolancea | 382 | MTNRIALRARANQFFVYSGRSLLVTNLDGDVSLSGTNGFY |
| | hollandica Lb | | RENTRLLSRYELTVNGYRLVPVVASPVGGAGMLTYQQMP |
| | | | DEVGVPERSVAVEIAYFLSEGMRTVLRIENNWGVKSHQP |
| | | | DPPTIDLAINVAADFADISEADTGTRLQKAPVQASWDDTR |
| | | | RELCFRYCHPKLDRAVAIRVEQSPVPVRYEQDALIVSLTLA |
| | | | LHQPVELVLVVEPVFDGKRLTAPPPVYDETETGLGRLRQR |
| | | | LRDETPRLTTSNATVARAWETAVSDLATLPLGRDPGPAAP |
| | | | IAGLPLYQQFFGRDTLTIGWQSLIAMPAMLRDALRLNAAW |
| | | | QGTVIDDFYDEEPGKLIHQASRAPLAILGYNPFLHYYGDYA |
| | | | TPPDFLLGLGQYAAWTSDRETVRPLLENARKAIDWLDRY |
| | | | GDLDHDGFLEYVCRSPEGLKNQGWKDSWDGIIDQDGNV |
| | | | VPDPIAACELQGYWYVGLQQVAIAFLLAGDLPYALELLNK |
| | | | ARDLKARFDPAFWMEDEGFYALGLGPDKEQIRSIASNAG |
| | | | HLLAAGIVPAEKAPRVARRLMEPDMFSGWGIRTLSSHHVA |
| | | | YNPFSYHLGSVWPVESGTIAFGMARYSCWPECHRLAGG |
| | | | LFALTERCSESRLPEVVGGIQRDEAHPYPGIYPGSNEPQG |
| | | | WSDGAIVVTIQALLGMHGLAPLGLLLVDPHLPDWLPDLRL |
| | | | NGLRVGESELDLDFVRTKSGKTRYHVRRQQGHIRVVHQP |
| | | | VPSEPHATLIGRLLDALGSIGHS |
| CCF83107.1 | Nitrolancea | 383 | MTNRIALRARANQFFVYSGRSLLVTNLDGDVSLSGTNGFY |
| (6X-His) | hollandica Lb | | RENTRLLSRYELTVNGYRLVPVVASPVGGAGMLTYQQMP |
| | | | DEVGVPERSVAVEIAYFLSEGMRTVLRIENNWGVKSHQP |
| | | | DPPTIDLAINVAADFADISEADTGTRLQKAPVQASWDDTR |
| | | | RELCFRYCHPKLDRAVAIRVEQSPVPVRYEQDALIVSLTLA |
| | | | LHQPVELVLVVEPVFDGKRLTAPPPVYDETETGLGRLRQR |
| | | | LRDETPRLTTSNATVARAWETAVSDLATLPLGRDPGPAAP |
| | | | IAGLPLYQQFFGRDTLTIGWQSLIAMPAMLRDALRLNAAW |
| | | | QGTVIDDFYDEEPGKLIHQASRAPLAILGYNPFLHYYGDYA |
| | | | TPPDFLLGLGQYAAWTSDRETVRPLLENARKAIDWLDRY |
| | | | GDLDHDGFLEYVCRSPEGLKNQGWKDSWDGIIDQDGNV |
| | | | VPDPIAACELQGYWYVGLQQVAIAFLLAGDLPYALELLNK |
| | | | ARDLKARFDPAFWMEDEGFYALGLGPDKEQIRSIASNAG |
| | | | HLLAAGIVPAEKAPRVARRLMEPDMFSGWGIRTLSSHHVA |
| | | | YNPFSYHLGSVWPVESGTIAFGMARYSCWPECHRLAGG |
| | | | LFALTERCSESRLPEVVGGIQRDEAHPYPGIYPGSNEPQG |
| | | | WSDGAIVVTIQALLGMHGLAPLGLLLVDPHLPDWLPDLRL |
| | | | NGLRVGESELDLDFVRTKSGKTRYHVRRQQGHIRVVHQP |
| | | | VPSEPHATLIGRLLDALGSIGHSHHHHHH |
| CCF84768.1 | Nitrolancea | 384 | MSIADYLSQVPLFQGLSACERVKLAESMRRHTYAPNDVIV |
| | hollandica Lb | | RQGDKTTDFYIILSGSVRVSRKAGRREIPIAELHAGDFFGD |
| | | | MALLDSLPRSATVTAIEPTECLSLSWEAFHRDVLGKPEVA |
| | | | RAMLSTLSRRVRDVEDLAFQPQTQSPATAREREDRLLRE |
| | | | GESSLITDIRETLTIKSGNLFLLCDEEGNIPIGNTAGLGLYLG |
| | | | DTRHLSGYEMILDQIRPTTLLTSARLGSASEQQLTNRDIRI |
| | | | RNRIVRKETLLISRSRVIGSGLEERLVVSNFNSFEVTITLLL |
| | | | RFASDFADIFEVRGLRRDQRGSIHRPTVDPVGVTLSYDGL |
| | | | DHVRRETRLEFSPAPDRVSGTSAVFRLRLPPLGSVSLRIT |
| | | | VTASHSSLHPVARDQSRPLMPPPGQGAGWLAAQTNIETN |
| | | | NEFFNAALDRSLADLRLLINRLDDDEYIAAGTPWYASLFGR |
| | | | DSLITAWQTLAWNPEIARGVLRLLARFQGRQRDDWRDEE |
| | | | PGKILHELRTGELAKSGLIPHSPYYGAVDSTPLFVMLLGAY |
| | | | ERWTGDQALVEQLAPALDAALQWMIQDGDPDHDGFIEYS |
| | | | RRSIKGLTNQGWKDSGEGIVNINGSLPQPPIALVEVQGY |
| | | | AYAAYLAGADLLETLGRTGEAGNLRARAVLLRDQFEQQF |
| | | | WMETERYYALGLDGRKQHITAITSNPGHALWTGIVSQERA |
| | | | ELVADRLLSPALFSGWGVRTLAEDMAAYNPLGYHLGTIW |
| | | | PHDNALIVAGLMRYGCSDHARRIATSLFDAGRAFRYYRLP |
| | | | ELFCGITRTEHSTPIGYPVACSPQAWAAGSLPYILQALLGI |
| | | | HPQSSGRVLLVDHPALPDWLREVRVNRLRIGSGRVNLVA |
| | | | RREGPRTAVDVLAVDGDVAVDVRLGG |
| CCF84768.1 | Nitrolancea | 385 | MSIADYLSQVPLFQGLSACERVKLAESMRRHTYAPNDVIV |
| (6X-His) | hollandica Lb | | RQGDKTTDFYIILSGSVRVSRKAGRREIPIAELHAGDFFGD |
| | | | MALLDSLPRSATVTAIEPTECLSLSWEAFHRDVLGKPEVA |
| | | | RAMLSTLSRRVRDVEDLAFQPQTQSPATAREREDRLLRE |
| | | | GESSLITDIRETLTIKSGNLFLLCDEEGNIPIGNTAGLGLYLG |
| | | | DTRHLSGYEMILDQIRPTTLLTSARLGSASEQQLTNRDIRI |
| | | | RNRIVRKETLLISRSRVIGSGLEERLVVSNFNSFEVTITLLL |
| | | | RFASDFADIFEVRGLRRDQRGSIHRPTVDPVGVTLSYDGL |
| | | | DHVRRETRLEFSPAPDRVSGTSAVFRLRLPPLGSVSLRIT |
| | | | VTASHSSLHPVARDQSRPLMPPPGQGAGWLAAQTNIETN |
| | | | NEFFNAALDRSLADLRLLINRLDDDEYIAAGTPWYASLFGR |
| | | | DSLITAWQTLAWNPEIARGVLRLLARFQGRQRDDWRDEE |
| | | | PGKILHELRTGELAKSGLIPHSPYYGAVDSTPLFVMLLGAY |
| | | | ERWTGDQALVEQLAPALDAALQWMIQDGDPDHDGFIEYS |
| | | | RRSIKGLTNQGWKDSGEGIVNINGSLPQPPIALVEVQGY |
| | | | AYAAYLAGADLLETLGRTGEAGNLRARAVLLRDQFEQQF |
| | | | WMETERYYALGLDGRKQHITAITSNPGHALWTGIVSQERA |
| | | | ELVADRLLSPALFSGWGVRTLAEDMAAYNPLGYHLGTIW |
| | | | PHDNALIVAGLMRYGCSDHARRIATSLFDAGRAFRYYRLP |
| | | | ELFCGITRTEHSTPIGYPVACSPQAWAAGSLPYILQALLGI |
| | | | HPQSSGRVLLVDHPALPDWLREVRVNRLRIGSGRVNLVA |
| | | | RREGPRTAVDVLAVDGDVAVDVRLGGHHHHHH |
| AFW94471.1 | Anabaena sp. 90 | 386 | MTPDTLMTPETIVLDNRTFTPADQKEISEWPCVVSQRPQP |
| | | | TITVKDDDLFLVTDTMGNISDCYLYGSTPSMGMFCRDTRF |
| | | | LSRLELQIEGRSPILLSSNADKGFALSVLCTNPDLENLFKA |
| | | | DTVGIRREIVLNGALFEEIEVANYNTTSIVFELSLSFDADFV |
| | | | DLFEVRGHHRQKRGRLLHLNELTSDHVASSSPQTHKEES |
| | | | LTLAYQGLDASIMESLIQFQHLQPTRFHGSTAVWRIELASH |
| | | | ETKKLGYRLNMFTDNQPSSTISAASTLAQAKAAELMEEQN |
| | | | WLQQITRISSDKSLFNRVISRAEQDIYLLLQSFGKHKTVSA |
| | | | GVPWFSTLFGRDSIITASQTLMLNSKIAKETLILLAEYQGQT |
| | | | DNIWREEEPGKILHELRLGEMARCQEIPHTPYYGTVDATP |
| | | | LWLMLYAEYYSWTHDQETLEALWPNALAAMGWIDRQLQ |
| | | | ETGYLSYARKSKGGLINQGWKDSGDCIVNRKGELAIGPIS |
| | | | LCEVQAYVYAAKNRLAEIAKIQQRPDLEELWQTEARQLRA |
| | | | KFNQDFWLEDQDFCALALDGEGKPVDSISSNPGQCLNLG |
| | | | IFTSAKAYSVAERLQAADMFNGWGIRTLSSLSPAYNPMGY |
| | | | HTGSVWPHDNSMIAMGLRSLGLIDQSLELFQGLFNMTSQ |
| | | | QPYQRPPELFCGYEMNGDNAPVQYPVACTPQAWATGSI |
| | | | FQLLQMMVNLVPDAQNNCLRIIDPALPESINNLSFHNLRVG |
| | | | GTILDLEFERIGSTTACRVAKKRGNLRVVIEA |
| AFW94471.1 | Anabaena sp. 90 | 387 | MTPDTLMTPETIVLDNRTFTPADQKEISEWPCVVSQRPQP |
| (6X-His) | | | TITVKDDDLFLVTDTMGNISDCYLYGSTPSMGMFCRDTRF |
| | | | LSRLELQIEGRSPILLSSNADKGFALSVLCTNPDLENLFKA |
| | | | DTVGIRREIVLNGALFEEIEVANYNTTSIVFELSLSFDADFV |
| | | | DLFEVRGHHRQKRGRLLHLNELTSDHVASSSPQTHKEES |
| | | | LTLAYQGLDASIMESLIQFQHLQPTRFHGSTAVWRIELASH |
| | | | ETKKLGYRLNMFTDNQPSSTISAASTLAQAKAAELMEEQN |
| | | | WLQQITRISSDKSLFNRVISRAEQDIYLLLQSFGKHKTVSA |
| | | | GVPWFSTLFGRDSIITASQTLMLNSKIAKETLILLAEYQGQT |
| | | | DNIWREEEPGKILHELRLGEMARCQEIPHTPYYGTVDATP |
| | | | LWLMLYAEYYSWTHDQETLEALWPNALAAMGWIDRQLQ |
| | | | ETGYLSYARKSKGGLINQGWKDSGDCIVNRKGELAIGPIS |
| | | | LCEVQAYVYAAKNRLAEIAKIQQRPDLEELWQTEARQLRA |
| | | | KFNQDFWLEDQDFCALALDGEGKPVDSISSNPGQCLNLG |
| | | | IFTSAKAYSVAERLQAADMFNGWGIRTLSSLSPAYNPMGY |
| | | | HTGSVWPHDNSMIAMGLRSLGLIDQSLELFQGLFNMTSQ |
| | | | QPYQRPPELFCGYEMNGDNAPVQYPVACTPQAWATGSI |
| | | | FQLLQMMVNLVPDAQNNCLRIIDPALPESINNLSFHNLRVG |
| | | | GTILDLEFERIGSTTACRVAKKRGNLRVVIEAHHHHHH |
| AAW86543.1 | Aliivibrio | 388 | MKISLSKANFRDMNRLEESNYLLTNGLGGYSSLTLAGSCS |
| | fischeri | | RNDHALFMGSLVAPNSRFCFVRHLDDRLTLANGTSHPLS |
| | (strain ATCC | | SQAKVNPYDTVDSSLLFENFSQTYLPQYTYRIDGIEITKTIV |
| | 700601/ES114) | | MAQGVNRLGVEYRVFNPLKHPFTLDITPKYHFCAKGEALS |
| | | | KEQAFIHQEGKVSSNGQTLHYRHNGSNTALSAPSYESGL |
| | | | YFSYDARDGRSASDAQVALHTITFDGTESNESFFVIFEAE |
| | | | EERAHYLAPSSIETMINDEVQRQQACITQSGLKSTFAQQL |
| | | | VRASDQFVVMRDSTESKSIIAGYPFFADWGRDTMIALWG |
| | | | CNLSTNQFEDAKQIFRTFIRYEHKGILPNLFPEAGVEPMYN |
| | | | TIDAALLFVVSLYEYYQASNDLDFIRDEAYDCMVNIYQHYR |
| | | | NGTDFDIKMLDNGLISGGSGLDQLTWMDVRYGDILPTPR |
| | | | QGCAVEINALWYNALSILHFFNQKLGKSMPELPALTEQVK |
| | | | ETFNHAFYNDKGYLNDWIKGEEVNQQIRPNQAYAVGLPF |
| | | | PILSEEKARSVILNIYQHLYTPLGLRSLAMQDPEFKSVHTG |
| | | | SHYDRDMAYHQGTVWGFMIGQFLMGILRHFPNDASLMHI |
| | | | VEQFISHNQDALSEGCIGQLAEIYDGYSPNQSRGCFAQA |
| | | | WSVSEVLRVLKHYEERI |
| AAW86543.1 | Aliivibrio | 389 | MKISLSKANFRDMNRLEESNYLLTNGLGGYSSLTLAGSCS |
| (6X-His) | fischeri | | RNDHALFMGSLVAPNSRFCFVRHLDDRLTLANGTSHPLS |
| | (strain ATCC | | SQAKVNPYDTVDSSLLFENFSQTYLPQYTYRIDGIEITKTIV |
| | 700601/ES114) | | MAQGVNRLGVEYRVFNPLKHPFTLDITPKYHFCAKGEALS |
| | | | KEQAFIHQEGKVSSNGQTLHYRHNGSNTALSAPSYESGL |
| | | | YFSYDARDGRSASDAQVALHTITFDGTESNESFFVIFEAE |
| | | | EERAHYLAPSSIETMINDEVQRQQACITQSGLKSTFAQQL |
| | | | VRASDQFVVMRDSTESKSIIAGYPFFADWGRDTMIALWG |
| | | | CNLSTNQFEDAKQIFRTFIRYEHKGILPNLFPEAGVEPMYN |
| | | | TIDAALLFVVSLYEYYQASNDLDFIRDEAYDCMVNIYQHYR |
| | | | NGTDFDIKMLDNGLISGGSGLDQLTWMDVRYGDILPTPR |
| | | | QGCAVEINALWYNALSILHFFNQKLGKSMPELPALTEQVK |
| | | | ETFNHAFYNDKGYLNDWIKGEEVNQQIRPNQAYAVGLPF |
| | | | PILSEEKARSVILNIYQHLYTPLGLRSLAMQDPEFKSVHTG |
| | | | SHYDRDMAYHQGTVWGFMIGQFLMGILRHFPNDASLMHI |
| | | | VEQFISHNQDALSEGCIGQLAEIYDGYSPNQSRGCFAQA |
| | | | WSVSEVLRVLKHYEERIHHHHHH |
| AAN69645.1 | Pseudomonas | 390 | MSPRTPKKTRSVAPSRIREGMPFPLGATWDGLGVNFALF |
| | putida (strain | | SANATKVELCLFDSTGEQELERIELPEYTDEIYHGYLPDAH |
| | ATCC 47054/ | | PGLVYGYRVYGPYEPENGHRFNPNKLLIDPYAKQLVGSLK |
| | DSM 6125/ | | WSEALFGYTIGHPDGDLSFDERDSAPFVPKCKVIDPAFTW |
| | CFBP 8728/ | | GRDQRVQIPWERTIIYEAHARGISMRHPAVPEELRGTFAG |
| | NCIMB 11950/ | | LANDELLKHIKDLGVSSIELLPIHAFVNDQHLLDKGLNNYW |
| | KT2440) | | GYNSIAFFAPHPRYLASGKIAEFKEMVAHLHDAGLEVILDV |
| | | | VYNHTAEGNERGPTLSMRGIDNASYYRLMPDDKRYYIND |
| | | | SGTGNTLDLSHPCVLQLVTDSLRYWAGEMHVDGFRFDLA |
| | | | TILGRYHDGYSERHGFLVACRQDPMLSQVKLIAEPWDCG |
| | | | PGGYQVGNFAPGWAEWNDRFRDTARAFWKGDEGQLAD |
| | | | FAARLTASGDMFNNRGRRPYSSVNFITAHDGFTLRDLVSY |
| | | | NHKHNEDNDENNQDGTDNNLSWNCGVEGPTDDPAINAL |
| | | | RMRQMRNYFATLLLAQGTPMIVAGDEFSRTQHGNNNAY |
| | | | CQDSEIGWVNWDLDEEGQELLAFVKRLTRLRLAYPVLRR |
| | | | SRFLVGDYNEAIGVKDVTWLAPDGSEMSVEQWEDPHGR |
| | | | CLGMLIDGRAQVSGIARPGAEATVLLIVNAHHDIVPFKLPA |
| | | | VPEGDYWSCLVDTDRPELRKGQHLQFDSTFEVKGRSMLL |
| | | | MVLQYDEE |
| AAN69645.1 | Pseudomonas | 391 | MSPRTPKKTRSVAPSRIREGMPFPLGATWDGLGVNFALF |
| (6X-His) | putida (strain | | SANATKVELCLFDSTGEQELERIELPEYTDEIYHGYLPDAH |
| | ATCC 47054/ | | PGLVYGYRVYGPYEPENGHRFNPNKLLIDPYAKQLVGSLK |
| | DSM 6125/ | | WSEALFGYTIGHPDGDLSFDERDSAPFVPKCKVIDPAFTW |
| | CFBP 8728/ | | GRDQRVQIPWERTIIYEAHARGISMRHPAVPEELRGTFAG |
| | NCIMB 11950/ | | LANDELLKHIKDLGVSSIELLPIHAFVNDQHLLDKGLNNYW |
| | KT2440) | | GYNSIAFFAPHPRYLASGKIAEFKEMVAHLHDAGLEVILDV |
| | | | VYNHTAEGNERGPTLSMRGIDNASYYRLMPDDKRYYIND |
| | | | SGTGNTLDLSHPCVLQLVTDSLRYWAGEMHVDGFRFDLA |
| | | | TILGRYHDGYSERHGFLVACRQDPMLSQVKLIAEPWDCG |
| | | | PGGYQVGNFAPGWAEWNDRFRDTARAFWKGDEGQLAD |
| | | | FAARLTASGDMFNNRGRRPYSSVNFITAHDGFTLRDLVSY |
| | | | NHKHNEDNDENNQDGTDNNLSWNCGVEGPTDDPAINAL |
| | | | RMRQMRNYFATLLLAQGTPMIVAGDEFSRTQHGNNNAY |
| | | | CQDSEIGWVNWDLDEEGQELLAFVKRLTRLRLAYPVLRR |
| | | | SRFLVGDYNEAIGVKDVTWLAPDGSEMSVEQWEDPHGR |
| | | | CLGMLIDGRAQVSGIARPGAEATVLLIVNAHHDIVPFKLPA |
| | | | VPEGDYWSCLVDTDRPELRKGQHLQFDSTFEVKGRSMLL |
| | | | MVLQYDEEHHHHHH |
| ARBA000 | Candida glabrata | 392 | MSAHRTLLLRLSDSGEPVTSCSYGQGVLTLPSLPLPQGKK |
| 20723 | (strain ATCC | | LGDMPVYTVKLAIPAGSPVTRDGLIWTNCPPDFSTQFDRE |
| (6X-His) | 2001/CBS 138/ | | KFYKKIIKTSFHEDDHIDLDIYVPGTYCFYLSFKNDKDELET |
| | JCM 3761/ | | TRKFYFVVLPILSVNDKFIPLNSIAMQSVVSKWMGPTIKDW |
| | NBRC 0622/ | | EKVFARVASKKYNMIHFTPLQHRGESNSPYSIYDQLEFDP |
| | NRRL Y-65) | | TVFKSEKEVADMVERLRTEHNILSLTDIVFNHTANNSQWL |
| | | | LDHPEAGYNHKTSPHLISAIELDKKLLDFSEQMEALGYPVD |
| | | | LKTVDDLIKVMDGIKEHVIGELKLWEFYVVDVKQTVSELRE |
| | | | KWGNSKSWSDDNIPSKDDSTNLAQFVRDNATEPGFGSL |
| | | | GERGSNKINIDKFAAILKKLHSEDYNNGIEELATKILNDINLP |
| | | | FYKEYDDDINEVLEQLFNRIKYLRIDDHGPKQGPITKKLPLS |
| | | | EPYFTRFKAKDGEEYALANNGWIWDGNPLVDFASSQSKA |
| | | | YLRREVIVWGDCVKLRYGKGPSDSPYLWERMSKYVEMN |
| | | | ARIFNGFRIDNCHSTPLHVGQYFLDVARRVNPNLYVVAEL |
| | | | FSGSEAMDCLFVERLGISSLIREAMQAWSEEELSRLVHRH |
| | | | GGRPIGSYKFVPLDDFPYPADVKIDEEYCAYNPDDHSVKC |
| | | | VSEIMIPKTLTATPPHALFMDCTHDNETPNQKRTVEDTLP |
| | | | NAALVAFCSSAIGSVYGYDEVFPQLLDLVQEKRTYSCAEN |
| | | | TGISKVKTLLNNMREEIASEAVDIEDSEMHVHHDGQYITFH |
| | | | RTNAKNGKGWYLVARTKFHSSGDQMLPRIKLSQTKATFK |
| | | | AAFSLERTGDAPISDEIIEGIPTKLRELTGFDIGFDENTKET |
| | | | SILLPQDFPQGSIVIFETQQLGIDDSLDHFIRSGAIKATEKLS |
| | | | LESINYVLYRAEQEEYDYSEGRSGAYDIPDYGKPVYCGLQ |
| | | | GWVSILRKIIFYNDLAHPLSNNLRNGHWAVDYVVNRLDLY |
| | | | KDKEGVAEVQEWLRSRMERIKQLPSYLVPSFFALVVGIMY |
| | | | GCCRLRAMQLMSDNVGKSTVFVQSLAMTSIQMVSAMKS |
| | | | TSILPDQNIAAMAAGLPHFSTNYMRCWGRDVFISLRGLLL |
| | | | TTGRYEEAKEHILAFAKTLKHGLIPNLLDAGRNPRYNARDA |
| | | | AWFFVQAIQDYVTIVPGGVSLLQEKVTRRFPLDDEYIPYD |
| | | | DPKAFSYSSTIEEIIYEILNRHAGGIKYREANAGPNLDRVMK |
| | | | DEGFNVEVNVDWETGLIHGGSQFNCGTWMDKMGESEKA |
| | | | NSVGVPGTPRDGAAVEINGLLKSCLRFVLQLSKDGKFKYT |
| | | | EVTKPDGSKISLSSWNDLLQENFERCFYVPKNKEDDNKF |
| | | | EIDATIINRRGIYKDLYRSGKPYEDYQFRPNFTIAMVVAPEL |
| | | | FTPDYAAGAIELADQVLRGPVGMRTLDPSDYNYRPYYNN |
| | | | GEDSDDFATSKGRNYHQGPEWVWCYGYFIRAYHYFNFL |
| | | | TNPKCQVEGSAKKLKPSSYLYRKLYSRLLKHREWIENSP |
| | | | WAGLAELTNKDGEVCNDSSPTQAWSTGCLLDLFYDLWIS |
| | | | YEEHHHHHH |

Additional sequences were identified that may be useful in the compositions and methods described herein. In some embodiments, the composition includes an α-amylase that includes an amino acid sequence having at least 85% (e.g., at least 90%, 95%, 97%, 99%, or 100%) sequence identity to of any one of SEQ ID NOs: 393-445 as shown in Table 5 below.

TABLE 5
α-amylase Sequences

| | | SEQ | |
|---|---|---|---|
| NCBI ID | Species | ID NO: | Sequence |
| WP_017474613.1 | Bacillus | 393 | MANLNGTLMQYFEWYMPNDGQHWKRLQNDSAYLAEHGI |
| | licheniformis | | TAVWIPPAYKGTSQADVGYGAYDLYDLGEFHQKGTVRTK |
| | | | YGTKGELQSAIKSLHSRDINVYGDVVINHKGGADATEDVT |
| | | | AVEVDPADRNRVISGEHLIKAWTHFHFPGRGSTYSDFKW |
| | | | HWYHFDGTDWDESRKLNRIYKFQGKAWDWEVSNENGN |
| | | | YDYLMYADIDYDHPDVAAEIKRWGTWYANELQLDGFRLD |
| | | | AVKHIKFSFLRDWVNHVREKTGKEMFTVAEYWQNDLGAL |
| | | | ENYLNKTNFNHSVFDVPLHYQFHAASTQGGGYDMRKLLN |
| | | | GTVVSKHPLKSVTFVDNHDTQPGQSLESTVQTWFKPLAY |
| | | | AFILTRESGYPQVFYGDMYGTKGDSQREIPALKHKIEPILK |
| | | | ARKQYAYGAQHDYFDHHDIVGWTREGDSSVANSGLAALI |
| | | | TDGPGGAKRMYVGRQNAGETWHDITGNRSEPVVINSEG |
| | | | WGEFHVNGGSVSIYVQR |
| WP_017474613.1 | Bacillus | 394 | MANLNGTLMQYFEWYMPNDGQHWKRLQNDSAYLAEHGI |
| (6X-His) | licheniformis | | TAVWIPPAYKGTSQADVGYGAYDLYDLGEFHQKGTVRTK |
| | | | YGTKGELQSAIKSLHSRDINVYGDVVINHKGGADATEDVT |
| | | | AVEVDPADRNRVISGEHLIKAWTHFHFPGRGSTYSDFKW |
| | | | HWYHFDGTDWDESRKLNRIYKFQGKAWDWEVSNENGN |
| | | | YDYLMYADIDYDHPDVAAEIKRWGTWYANELQLDGFRLD |
| | | | AVKHIKFSFLRDWVNHVREKTGKEMFTVAEYWQNDLGAL |
| | | | ENYLNKTNFNHSVFDVPLHYQFHAASTQGGGYDMRKLLN |
| | | | GTVVSKHPLKSVTFVDNHDTQPGQSLESTVQTWFKPLAY |
| | | | AFILTRESGYPQVFYGDMYGTKGDSQREIPALKHKIEPILK |
| | | | ARKQYAYGAQHDYFDHHDIVGWTREGDSSVANSGLAALI |
| | | | TDGPGGAKRMYVGRQNAGETWHDITGNRSEPVVINSEG |
| | | | WGEFHVNGGSVSIYVQRHHHHHH |
| AGJ520811 | Rhizomucor | 395 | MATSDDWKSKAIYQLLTDRFGRADDSTSNCSNLSNYCGG |
| | pusillus | | TYEGITKHLDYISGMGFDAIWISPIPKNSDGGYHGYWATD |
| | | | FYQLNSNFGDESQLKALIQAAHERDMYVMLDVVANHAGP |
| | | | TSNGYSGYTFGDASLYHPKCTIDYNDQTSIEQCWVADELP |
| | | | DIDTENSDNVAILNDIVSGWVGNYSFDGIRIDTVKHIRKDF |
| | | | WTGYAEAAGVFATGEVFNGDPAYVGPYQKYLPSLINYPM |
| | | | YYALNDVFVSKSKGFSRISEMLGSNRNAFEDTSVLTTFVD |
| | | | NHDNPRFLNSQSDKALFKNALTYVLLGEGIPIVYYGSEQG |
| | | | FSGGADPANREVLWTTNYDTSSDLYQFIKTVNSVRMKSN |
| | | | KAVYMDIYVGDNAYAFKHGDALVVLNNYGSGSTNQVSFS |
| | | | VSGKFDSGASLMDIVSNITTTVSSDGTVTFNLKDGLPAIFT |
| | | | SA |
| AGJ52081.1 | Rhizomucor | 396 | MATSDDWKSKAIYQLLTDRFGRADDSTSNCSNLSNYCGG |
| (6X-His) | pusillus | | TYEGITKHLDYISGMGFDAIWISPIPKNSDGGYHGYWATD |
| | | | FYQLNSNFGDESQLKALIQAAHERDMYVMLDVVANHAGP |
| | | | TSNGYSGYTFGDASLYHPKCTIDYNDQTSIEQCWVADELP |
| | | | DIDTENSDNVAILNDIVSGWVGNYSFDGIRIDTVKHIRKDF |
| | | | WTGYAEAAGVFATGEVFNGDPAYVGPYQKYLPSLINYPM |
| | | | YYALNDVFVSKSKGFSRISEMLGSNRNAFEDTSVLTTFVD |
| | | | NHDNPRFLNSQSDKALFKNALTYVLLGEGIPIVYYGSEQG |
| | | | FSGGADPANREVLWTTNYDTSSDLYQFIKTVNSVRMKSN |
| | | | KAVYMDIYVGDNAYAFKHGDALVVLNNYGSGSTNQVSFS |
| | | | VSGKFDSGASLMDIVSNITTTVSSDGTVTFNLKDGLPAIFT |
| | | | SAHHHHHH |
| AFD54462.1 | Malbranchea | 397 | ATPDEWRSRSIYQVLTDRFARGDGSPDAPCDTGARKYC |
| | cinnamomea | | GGNYRGLISQLDYIQGMGFDSVWISPITKQFEDDWNGAP |
| | | | YHGYWQTDLYALNEHFGTEEDLRALADELHARGMFLMVD |
| | | | VVINHNGWPGDAASIDYSQFNPFNSSDYYHPPCEINYDD |
| | | | QTSVEQCWLYTGANALPDLKTEDPHVSQVHNDWIADLVS |
| | | | KYSIDGLRIDTTKHVDKPAIGSFNDAAGVYAVGEVYHGDP |
| | | | AYTCPYQDWVDGVLNFPVYYPLIDAFKSPSGTMWSLVDN |
| | | | INKVFQTCNDPRLLGTFSENHDIPRFASYTQDLALAKNVLA |
| | | | FTILFDGIPIVYAGQEQQYSGDSDPYNREALWLSGFNTDA |
| | | | PLYKHIAACNRIRSHAVSNDDAYITTPTDIKYSDDHTLALVK |
| | | | GAVTTVLTNAGANAGETTVTVEATGYASGEQVTDVLSCE |
| | | | SIAASDGGRLSVTLNQGLPRVFFPTDALAGSGLCEN |
| AFD54462.1 | Malbranchea | 398 | ATPDEWRSRSIYQVLTDRFARGDGSPDAPCDTGARKYC |
| (6X-His) | cinnamomea | | GGNYRGLISQLDYIQGMGFDSVWISPITKQFEDDWNGAP |
| | | | YHGYWQTDLYALNEHFGTEEDLRALADELHARGMFLMVD |
| | | | VVINHNGWPGDAASIDYSQFNPFNSSDYYHPPCEINYDD |
| | | | QTSVEQCWLYTGANALPDLKTEDPHVSQVHNDWIADLVS |
| | | | KYSIDGLRIDTTKHVDKPAIGSFNDAAGVYAVGEVYHGDP |
| | | | AYTCPYQDWVDGVLNFPVYYPLIDAFKSPSGTMWSLVDN |
| | | | INKVFQTCNDPRLLGTFSENHDIPRFASYTQDLALAKNVLA |
| | | | FTILFDGIPIVYAGQEQQYSGDSDPYNREALWLSGFNTDA |
| | | | PLYKHIAACNRIRSHAVSNDDAYITTPTDIKYSDDHTLALVK |
| | | | GAVTTVLTNAGANAGETTVTVEATGYASGEQVTDVLSCE |
| | | | SIAASDGGRLSVTLNQGLPRVFFPTDALAGSGLCENHHH |
| | | | HHH |
| WP_003897929.1 | Mycolicibacterium | 399 | MEEHTQGSHVEAGIVEHPNAEDFGHARTLPTDTNWFKHA |
| | smegmatis | | VFYEVLVRAFYDSNADGIGDLRGLTEKLDYIKWLGVDCLW |
| | (strain ATCC | | LPPFYDSPLRDGGYDIRDFYKVLPEFGTVDDFVTLLDAAH |
| | 700084/ | | RRGIRIITDLVMNHTSDQHEWFQESRHNPDGPYGDFYVW |
| | mc(2)155) | | SDTSDRYPDARIIFVDTEESNWTFDPVRRQFYWHRFFSH |
| | | | QPDLNYDNPAVQEAMLDVLRFWLDLGIDGFRLDAVPYLF |
| | | | EREGTNCENLPETHAFLKRCRKAIDDEYPGRVLLAEANQ |
| | | | WPADVVAYFGDPDTGGDECHMAFHFPLMPRIFMAVRRE |
| | | | SRFPISEILAQTPPIPDTAQWGIFLRNHDELTLEMVTDEER |
| | | | DYMYAEYAKDPRMKANVGIRRRLAPLLENDRNQIELFTAL |
| | | | LLSLPGSPVLYYGDEIGMGDIIWLGDRDSVRTPMQWTPD |
| | | | RNAGFSKATPGRLYLPPNQDAVYGYHSVNVEAQLDSSSS |
| | | | LLNWTRNMLAVRSRHDAFAVGTFRELGGSNPSVLAYIRE |
| | | | VTRQQGDGGAKTDAVLCVNNLSRFPQPIELNLQQWAGYI |
| | | | PVEMTGYVEFPSIGQLPYLLTLPGHGFYWFQLREPDPEP |
| | | | GAQQ |
| WP_003897929.1 | Mycolicibacterium | 400 | EEHTQGSHVEAGIVEHPNAEDFGHARTLPTDTNWFKHAV |
| (6X-His) | smegmatis | | FYEVLVRAFYDSNADGIGDLRGLTEKLDYIKWLGVDCLWL |
| | (strain ATCC | | PPFYDSPLRDGGYDIRDFYKVLPEFGTVDDFVTLLDAAHR |
| | 700084/ | | RGIRIITDLVMNHTSDQHEWFQESRHNPDGPYGDFYVWS |
| | mc(2)155) | | DTSDRYPDARIIFVDTEESNWTFDPVRRQFYWHRFFSHQ |
| | | | PDLNYDNPAVQEAMLDVLRFWLDLGIDGFRLDAVPYLFER |
| | | | EGTNCENLPETHAFLKRCRKAIDDEYPGRVLLAEANQWP |
| | | | ADVVAYFGDPDTGGDECHMAFHFPLMPRIFMAVRRESRF |
| | | | PISEILAQTPPIPDTAQWGIFLRNHDELTLEMVTDEERDYM |
| | | | YAEYAKDPRMKANVGIRRRLAPLLENDRNQIELFTALLLSL |
| | | | PGSPVLYYGDEIGMGDIIWLGDRDSVRTPMQWTPDRNAG |
| | | | FSKATPGRLYLPPNQDAVYGYHSVNVEAQLDSSSSLLNW |
| | | | TRNMLAVRSRHDAFAVGTFRELGGSNPSVLAYIREVTRQ |
| | | | QGDGGAKTDAVLCVNNLSRFPQPIELNLQQWAGYIPVEM |
| | | | TGYVEFPSIGQLPYLLTLPGHGFYWFQLREPDPEPGAQQ |
| | | | HHHHHH |
| WP_017474613.1 | Bacillus | 401 | MKQQKRLYARLLTLLFALIFLLPHSAAAAANLNGTLMQYFE |
| | licheniformis | | WYMPNDGQHWKRLQNDSAYLAEHGITAVWIPPAYKGTS |
| | | | QADVGYGAYDLYDLGEFHQKGTVRTKYGTKGELQSAIKS |
| | | | LHSRDINVYGDVVINHKGGADATEDVTAVEVDPADRNRVI |
| | | | SGEHRIKAWTHFHFPGRGSTYSDFKWHWYHFDGTDWDE |
| | | | SRKLNRIYKFQGKAWDWEVSNENGNYDYLMYADIDYDHP |
| | | | DVAAEIKRWGTWYANELQLDGFRLDAVKHIKFSFLRDWV |
| | | | NHVREKTGKEMFTVAEYWQNDLGALENYLNKTNFNHSVF |
| | | | DVPLHYQFHAASTQGGGYDMRKLLNSTVVSKHPLKAVTF |
| | | | VDNHDTQPGQSLESTVQTWFKPLAYAFILTRESGYPQVF |
| | | | YGDMYGTKGDSQREIPALKHKIEPILKARKQYAYGAQHDY |
| | | | FDHHDIVGWTREGDSSVANSGLAALITDGPGGAKRMYVG |
| | | | RQNAGETWHDITGNRSEPVVINSEGWGEFHVNGGSVSIY |
| | | | VQR |
| WP_017474613.1 | Bacillus | 402 | AANLNGTLMQYFEWYMPNDGQHWKRLQNDSAYLAEHGI |
| (6X-His) | licheniformis | | TAVWIPPAYKGTSQADVGYGAYDLYDLGEFHQKGTVRTK |
| | | | YGTKGELQSAIKSLHSRDINVYGDVVINHKGGADATEDVT |
| | | | AVEVDPADRNRVISGEHRIKAWTHFHFPGRGSTYSDFKW |
| | | | HWYHFDGTDWDESRKLNRIYKFQGKAWDWEVSNENGN |
| | | | YDYLMYADIDYDHPDVAAEIKRWGTWYANELQLDGFRLD |
| | | | AVKHIKFSFLRDWVNHVREKTGKEMFTVAEYWQNDLGAL |
| | | | ENYLNKTNFNHSVFDVPLHYQFHAASTQGGGYDMRKLLN |
| | | | STVVSKHPLKAVTFVDNHDTQPGQSLESTVQTWFKPLAY |
| | | | AFILTRESGYPQVFYGDMYGTKGDSQREIPALKHKIEPILK |
| | | | ARKQYAYGAQHDYFDHHDIVGWTREGDSSVANSGLAALI |
| | | | TDGPGGAKRMYVGRQNAGETWHDITGNRSEPVVINSEG |
| | | | WGEFHVNGGSVSIYVQRHHHHHH |
| AAA25854.1 | Paenibacillus | 403 | MTLYRSLWKKGCMLLLSLVLSLTAFIGSPSNTASAAVADD |
| | polymyxa | | FQASVMGPLAKINDWGSFKKQLQTLKNNGVYAITTDVWW |
| | | | GYVESAGDNQFDWSYYKTYANAVKEAGLKWVPIISTHKC |
| | | | GGNVGDDCNIPLPSWLSSKGSADEMQFKDESGYANSEA |
| | | | LSPLWSGTGKQYDELYASFAENFAGYKSIIPKIYLSGGPSG |
| | | | ELRYPSYYPAAGWSYPGRGKFQAYTETAKNAFRTAMND |
| | | | KYGSLDKINAAWGTKLTSLSQINPPTDGDGFYTNGGYNSA |
| | | | YGKDFLSWYQSVLEKHLGVIGAAAHKNFDSVFGVRIGAKI |
| | | | SGLHWQMNNPAMPHGTEQAGGYYDYNRLIQKFKDADLD |
| | | | LTFTCLEMSDSGTAPNYSLPSTLVDTVSSIANAKGVRLNG |
| | | | ENALPTGGSGFQKIEEKITKFGYHGFTLLRINNLVNNDGSP |
| | | | TGELSGFKQYIISKAKPDNNGGTGNKVTIYYKKGFNSPYIH |
| | | | YRPAGGSWTAAPGVKMQDAEISGYAKITVDIGSASQLEAA |
| | | | FNDGNNNWDSNNTKNYSFSTGTSTYTPGNSGNAGTITSG |
| | | | APAGANPGDGGGTTNKVTVYYKKGFNSPYIHYRPAGGS |
| | | | WTAAPGVKMQDAEISGYAKITVDIGSASQLEAAFNDGNNN |
| | | | WDSNNTKNYLFSTGTSTYTPGSNGAAGTIRTGAPSGSVL |
| | | | SVVTSTYATDLNEVTGPIQTEKLSGVSLNVSTSTYAPNSN |
| | | | GVEVTAQTEAPSGAFTSMDLGTLSNPTSLNTDWSKQSIYF |
| | | | IMTDRFSNGDPSNDNYGGFNSNNSDQRKWHGGDFQGII |
| | | | NKLDYIKNMGFTAIWITPVTMQKSEYAYHGYHTYDFYAVD |
| | | | GHLGTMDKLQELVRKAHDKNIAVMVDVVVNHTGDFQPGN |
| | | | GFAKAPFDKADWYHHNGDITDGDYNSNNQWKIENGDVA |
| | | | GLDDLNHENPATANELKNWIKWLLNETGIDGLRLDTVKHV |
| | | | PKGFLKDFDQAANTFTMGEIFHGDPAYVGDYTRYLDAAL |
| | | | DFPMYYTIKDVFGHDQSMRKIKDRYSDDRYYRDAQTNGV |
| | | | FIDNHDVKRFLNDASGKPGANYDKWPQLKAALGFTLTSR |
| | | | GIPIIYQGTEQGYSGGDDPANRENMNFNANHDLYQYIAKL |
| | | | NYVRNNHPALQNGSQREKWVDDSFYSFQRSKNGDEAIV |
| | | | FINNSWNSQTRTIGNFDNLSNGTRLTNQLSNDSVQINNGS |
| | | | ITVTLAPKEVKVFTK |
| AAA25854.1 | Paenibacillus | 404 | AVADDFQASVMGPLAKINDWGSFKKQLQTLKNNGVYAITT |
| (6X-His) | polymyxa | | DVWWGYVESAGDNQFDWSYYKTYANAVKEAGLKWVPII |
| | | | STHKCGGNVGDDCNIPLPSWLSSKGSADEMQFKDESGY |
| | | | ANSEALSPLWSGTGKQYDELYASFAENFAGYKSIIPKIYLS |
| | | | GGPSGELRYPSYYPAAGWSYPGRGKFQAYTETAKNAFR |
| | | | TAMNDKYGSLDKINAAWGTKLTSLSQINPPTDGDGFYTN |
| | | | GGYNSAYGKDFLSWYQSVLEKHLGVIGAAAHKNFDSVFG |
| | | | VRIGAKISGLHWQMNNPAMPHGTEQAGGYYDYNRLIQKF |
| | | | KDADLDLTFTCLEMSDSGTAPNYSLPSTLVDTVSSIANAK |
| | | | GVRLNGENALPTGGSGFQKIEEKITKFGYHGFTLLRINNLV |
| | | | NNDGSPTGELSGFKQYIISKAKPDNNGGTGNKVTIYYKKG |
| | | | FNSPYIHYRPAGGSWTAAPGVKMQDAEISGYAKITVDIGS |
| | | | ASQLEAAFNDGNNNWDSNNTKNYSFSTGTSTYTPGNSG |
| | | | NAGTITSGAPAGANPGDGGGTTNKVTVYYKKGFNSPYIH |
| | | | YRPAGGSWTAAPGVKMQDAEISGYAKITVDIGSASQLEAA |
| | | | FNDGNNNWDSNNTKNYLFSTGTSTYTPGSNGAAGTIRTG |
| | | | APSGSVLSVVTSTYATDLNEVTGPIQTEKLSGVSLNVSTST |
| | | | YAPNSNGVEVTAQTEAPSGAFTSMDLGTLSNPTSLNTDW |
| | | | SKQSIYFIMTDRFSNGDPSNDNYGGFNSNNSDQRKWHG |
| | | | GDFQGIINKLDYIKNMGFTAIWITPVTMQKSEYAYHGYHTY |
| | | | DFYAVDGHLGTMDKLQELVRKAHDKNIAVMVDVVVNHTG |
| | | | DFQPGNGFAKAPFDKADWYHHNGDITDGDYNSNNQWKI |
| | | | ENGDVAGLDDLNHENPATANELKNWIKWLLNETGIDGLRL |
| | | | DTVKHVPKGFLKDFDQAANTFTMGEIFHGDPAYVGDYTR |
| | | | YLDAALDFPMYYTIKDVFGHDQSMRKIKDRYSDDRYYRD |
| | | | AQTNGVFIDNHDVKRFLNDASGKPGANYDKWPQLKAALG |
| | | | FTLTSRGIPIIYQGTEQGYSGGDDPANRENMNFNANHDLY |
| | | | QYIAKLNYVRNNHPALQNGSQREKWVDDSFYSFQRSKN |
| | | | GDEAIVFINNSWNSQTRTIGNFDNLSNGTRLTNQLSNDSV |
| | | | QINNGSITVTLAPKEVKVFTKHHHHHH |
| CAA41481.1 | Pseudoaltermonas | 405 | MKLNKIITTAGLSLGLLLPSIATATPTTFVHLFEWNWQDVA |
| | haloplanktis | | QECEQYLGPKGYAAVQVSPPNEHITGSQWWTRYQPVSY |
| | | | ELQSRGGNRAQFIDMVNRCSAAGVDIYVDTLINHMAAGS |
| | | | GTGTAGNSFGNKSFPIYSPQDFHESCTINNSDYGNDRYR |
| | | | VQNCELVGLADLDTASNYVQNTIAAYINDLQAIGVKGFRFD |
| | | | ASKHVAASDIQSLMAKVNGSPVVFQEVIDQGGEAVGASE |
| | | | YLSTGLVTEFKYSTELGNTFRNGSLAWLSNFGEGWGFMP |
| | | | SSSAVVFVDNHDNQRGHGGAGNVITFEDGRLYDLANVFM |
| | | | LAYPYGYPKVMSSYDFHGDTDAGGPNVPVHNNGNLECF |
| | | | ASNWKCEHRWSYIAGGVDFRNNTADNWAVTNWWDNTN |
| | | | NQISFGRGSSGHMAINKEDSTLTATVQTDMASGQYCNVL |
| | | | KGELSADAKSCSGEVITVNSDGTINLNIGAWDAMAIHKNA |
| | | | KLNTSSASSTESDWQRTVIFINAQTQSGQDMFIRGGIDHA |
| | | | YANANLGRNCQTSNFECAMPIRHNNLKNVTTSPWKANDN |
| | | | YLDWYGIENGQSSEAEGSATDWTTNVWPAGWGAEKTVN |
| | | | TDGFGVTPLNIWGEHYWMLDVDMDCSKAVNGWFELKAFI |
| | | | KNGQGWETAIAQDNAPYTSTNHMAQCGKINKFEFNNSGV |
| | | | VIRSF |
| CAA41481.1 | Pseudoalteromonas | 406 | TPTTFVHLFEWNWQDVAQECEQYLGPKGYAAVQVSPPN |
| (6X-His) | haloplanktis | | EHITGSQWWTRYQPVSYELQSRGGNRAQFIDMVNRCSA |
| | | | AGVDIYVDTLINHMAAGSGTGTAGNSFGNKSFPIYSPQDF |
| | | | HESCTINNSDYGNDRYRVQNCELVGLADLDTASNYVQNTI |
| | | | AAYINDLQAIGVKGFRFDASKHVAASDIQSLMAKVNGSPV |
| | | | VFQEVIDQGGEAVGASEYLSTGLVTEFKYSTELGNTFRNG |
| | | | SLAWLSNFGEGWGFMPSSSAVVFVDNHDNQRGHGGAG |
| | | | NVITFEDGRLYDLANVFMLAYPYGYPKVMSSYDFHGDTD |
| | | | AGGPNVPVHNNGNLECFASNWKCEHRWSYIAGGVDFRN |
| | | | NTADNWAVTNWWDNTNNQISFGRGSSGHMAINKEDSTL |
| | | | TATVQTDMASGQYCNVLKGELSADAKSCSGEVITVNSDG |
| | | | TINLNIGAWDAMAIHKNAKLNTSSASSTESDWQRTVIFINA |
| | | | QTQSGQDMFIRGGIDHAYANANLGRNCQTSNFECAMPIR |
| | | | HNNLKNVTTSPWKANDNYLDWYGIENGQSSEAEGSATD |
| | | | WTTNVWPAGWGAEKTVNTDGFGVTPLNIWGEHYWMLD |
| | | | VDMDCSKAVNGWFELKAFIKNGQGWETAIAQDNAPYTST |
| | | | NHMAQCGKINKFEFNNSGVVIRSFHHHHHH |
| AAB42174.1 | Bacteroides | 407 | MNKHLHFLSLLWLSMLMAFMTACSDDKNITDPAPEPEPP |
| | thetaiotaomicron | | VEGQWTALTASPDTWDETKRADISYQLLLYSFADSDGDG |
| | (strain ATCC | | YGDLNGVTQKLDYLNQLGVKALWLSPIHPCMSYHGYDVT |
| | 29148/DSM | | DYTKVNPQLGTESDFDRLVTEAHNRGIKIYLDYVMNHTGT |
| | 2079/JCM 5827/ | | AHPWFTEASSSSESPYRNYYSFSEDPKTDIAAGKIAMITQ |
| | CCUG 10774/ | | EGAAGYNAAEWFQVSDETAAVKGLLKFTLDWSNAPSPIL |
| | NCTC 10582/ | | VVSTGTKADEDNPDTGTDNAKYLYYGEDICKKFYDKGNNI |
| | VPI-5482/E50) | | YELTVDFESTWGLLIRTSNASFWPSGTKYGASSSSEKLAL |
| | | | NKDFKLTNAGNPANIMFDSQQITYFHSHFCTDWFADLNY |
| | | | GPVDQAGESPAYQAIADAAKGWIARGVDGLRLDAVKHIY |
| | | | HSETSEENPRFLKMFYEDMNAYYKQKGHTDDFYMIGEVL |
| | | | SEYDKVAPYYKGLPALFEFSFWYRLEWGINNSTGCYFAK |
| | | | DILSYQQKYANYRSDYIEATKLSNHDEDRTSSKLGKSADK |
| | | | CKLAAAVLLTSAGHPYIYYGEELGLYGTKDNGDEYVRSPM |
| | | | LWGDSYTTNYTDKTDATVSKNVKTVADQQADTHSLLNIYF |
| | | | SLTRLRNTYPALAEGNMTKHSVYNESQEKDYKPIAAWYM |
| | | | TKDNEKLLVIHNFGGTAMQLPLTDKIEKVLFVNGETQQNT |
| | | | DSDSYTLKLGGYASVVFKLGN |
| AAB42174.1 | Bacteroides | 408 | CSDDKNITDPAPEPEPPVEGQWTALTASPDTWDETKRAD |
| (6X-His) | thetaiotaomicron | | ISYQLLLYSFADSDGDGYGDLNGVTQKLDYLNQLGVKAL |
| | (strain ATCC | | WLSPIHPCMSYHGYDVTDYTKVNPQLGTESDFDRLVTEA |
| | 29148/DSM | | HNRGIKIYLDYVMNHTGTAHPWFTEASSSSESPYRNYYSF |
| | 2079/JCM 5827/ | | SEDPKTDIAAGKIAMITQEGAAGYNAAEWFQVSDETAAVK |
| | CCUG 10774/ | | GLLKFTLDWSNAPSPILVVSTGTKADEDNPDTGTDNAKYL |
| | NCTC 10582/ | | YYGEDICKKFYDKGNNIYELTVDFESTWGLLIRTSNASFW |
| | VPI-5482/E50) | | PSGTKYGASSSSEKLALNKDFKLTNAGNPANIMFDSQQIT |
| | | | YFHSHFCTDWFADLNYGPVDQAGESPAYQAIADAAKGWI |
| | | | ARGVDGLRLDAVKHIYHSETSEENPRFLKMFYEDMNAYY |
| | | | KQKGHTDDFYMIGEVLSEYDKVAPYYKGLPALFEFSFWY |
| | | | RLEWGINNSTGCYFAKDILSYQQKYANYRSDYIEATKLSN |
| | | | HDEDRTSSKLGKSADKCKLAAAVLLTSAGHPYIYYGEELG |
| | | | LYGTKDNGDEYVRSPMLWGDSYTTNYTDKTDATVSKNVK |
| | | | TVADQQADTHSLLNIYFSLTRLRNTYPALAEGNMTKHSVY |
| | | | NESQEKDYKPIAAWYMTKDNEKLLVIHNFGGTAMQLPLTD |
| | | | KIEKVLFVNGETQQNTDSDSYTLKLGGYASVVFKLGNHHH |
| | | | HHH |
| NP_388186.2 | Bacillus subtilis | 409 | MFAKRFKTSLLPLFAGFLLLFHLVLAGPAAASAETANKSNE |
| | (strain 168) | | LTAPSIKSGTILHAWNWSFNTLKHNMKDIHDAGYTAIQTSP |
| | | | INQVKEGNQGDKSMSNWYWLYQPTSYQIGNRYLGTEQE |
| | | | FKEMCAAAEEYGIKVIVDAVINHTTSDYAAISNEVKSIPNW |
| | | | THGNTQIKNWSDRWDVTQNSLLGLYDWNTQNTQVQSYL |
| | | | KRFLDRALNDGADGFRFDAAKHIELPDDGSYGSQFWPNI |
| | | | TNTSAEFQYGEILQDSASRDAAYANYMDVTASNYGHSIRS |
| | | | ALKNRNLGVSNISHYASDVSADKLVTWVESHDTYANDDE |
| | | | ESTWMSDDDIRLGWAVIASRSGSTPLFFSRPEGGGNGVR |
| | | | FPGKSQIGDRGSALFEDQAITAVNRFHNVMAGQPEELSN |
| | | | PNGNNQIFMNQRGSHGVVLANAGSSSVSINTATKLPDGR |
| | | | YDNKAGAGSFQVNDGKLTGTINARSVAVLYPDDIAKAPHV |
| | | | FLENYKTGVTHSFNDQLTITLRADANTTKAVYQINNGPETA |
| | | | FKDGDQFTIGKGDPFGKTYTIMLKGTNSDGVTRTEKYSFV |
| | | | KRDPASAKTIGYQNPNHWSQVNAYIYKHDGSRVIELTGS |
| | | | WPGKPMTKNADGIYTLTLPADTDTTNAKVIFNNGSAQVPG |
| | | | QNQPGFDYVLNGLYNDSGLSGSLPH |
| NP_388186.2 | Bacillus subtilis | 410 | SAETANKSNELTAPSIKSGTILHAWNWSFNTLKHNMKDIH |
| (6X-His) | (strain 168) | | DAGYTAIQTSPINQVKEGNQGDKSMSNWYWLYQPTSYQI |
| | | | GNRYLGTEQEFKEMCAAAEEYGIKVIVDAVINHTTSDYAAI |
| | | | SNEVKSIPNWTHGNTQIKNWSDRWDVTQNSLLGLYDWN |
| | | | TQNTQVQSYLKRFLDRALNDGADGFRFDAAKHIELPDDG |
| | | | SYGSQFWPNITNTSAEFQYGEILQDSASRDAAYANYMDV |
| | | | TASNYGHSIRSALKNRNLGVSNISHYASDVSADKLVTWVE |
| | | | SHDTYANDDEESTWMSDDDIRLGWAVIASRSGSTPLFFS |
| | | | RPEGGGNGVRFPGKSQIGDRGSALFEDQAITAVNRFHNV |
| | | | MAGQPEELSNPNGNNQIFMNQRGSHGVVLANAGSSSVSI |
| | | | NTATKLPDGRYDNKAGAGSFQVNDGKLTGTINARSVAVL |
| | | | YPDDIAKAPHVFLENYKTGVTHSFNDQLTITLRADANTTKA |
| | | | VYQINNGPETAFKDGDQFTIGKGDPFGKTYTIMLKGTNSD |
| | | | GVTRTEKYSFVKRDPASAKTIGYQNPNHWSQVNAYIYKH |
| | | | DGSRVIELTGSWPGKPMTKNADGIYTLTLPADTDTTNAKVI |
| | | | FNNGSAQVPGQNQPGFDYVLNGLYNDSGLSGSLPHHHH |
| | | | HHH |
| WP_013352208.1 | Bacillus | 411 | MIQKRKRTVSFRLVLMCTLLFVSLPITKTSAVNGTLMQYFE |
| | amyloliquefaciens | | WYTPNDGQHWKRLQNDAEHLSDIGITAVWIPPAYKGLSQ |
| | | | SDNGYGPYDLYDLGEFQQKGTVRTKYGTKSELQDAIGSL |
| | | | HSRNVQVYGDVVLNHKAGADATEDVTAVEVNPANRNQE |
| | | | TSEEYQIKAWTDFRFPGRGNTYSDFKWHWYHFDGADWD |
| | | | ESRKISRIFKFRGEGKAWDWEVSSENGNYDYLMYADVDY |
| | | | DHPDVVAETKKWGIWYANELSLDGFRIDAAKHIKFSFLRD |
| | | | WVQAVRQATGKEMFTVAEYWQNNAGKLENYLNKTSFNQ |
| | | | SVFDVPLHFNLQAASSQGGGYDMRRLLDGTVVSRHPEKA |
| | | | VTFVENHDTQPGQSLESTVQTWFKPLAYAFILTRESGYPQ |
| | | | VFYGDMYGTKGTSPKEIPSLKDNIEPILKARKEYAYGPQH |
| | | | DYIDHPDVIGWTREGDSSAAKSGLAALITDGPGGSKRMYA |
| | | | GLKNAGETWYDITGNRSDTVKIGSDGWGEFHVNDGSVSI |
| | | | YVQK |
| WP_013352208.1 | Bacillus | 412 | VNGTLMQYFEWYTPNDGQHWKRLQNDAEHLSDIGITAV |
| (6X-His) | amyloliquefaciens | | WIPPAYKGLSQSDNGYGPYDLYDLGEFQQKGTVRTKYGT |
| | | | KSELQDAIGSLHSRNVQVYGDVVLNHKAGADATEDVTAV |
| | | | EVNPANRNQETSEEYQIKAWTDFRFPGRGNTYSDFKWH |
| | | | WYHFDGADWDESRKISRIFKFRGEGKAWDWEVSSENGN |
| | | | YDYLMYADVDYDHPDVVAETKKWGIWYANELSLDGFRID |
| | | | AAKHIKFSFLRDWVQAVRQATGKEMFTVAEYWQNNAGKL |
| | | | ENYLNKTSFNQSVFDVPLHFNLQAASSQGGGYDMRRLLD |
| | | | GTVVSRHPEKAVTFVENHDTQPGQSLESTVQTWFKPLAY |
| | | | AFILTRESGYPQVFYGDMYGTKGTSPKEIPSLKDNIEPILK |
| | | | ARKEYAYGPQHDYIDHPDVIGWTREGDSSAAKSGLAALIT |
| | | | DGPGGSKRMYAGLKNAGETWYDITGNRSDTVKIGSDGW |
| | | | GEFHVNDGSVSIYVQKHHHHHH |
| AAA22235.2 | Geobacillus | 413 | MLTFHRIIRKGWMFLLAFLLTALLFCPTGQPAKAAAPFNGT |
| | stearothermophilus | | MMQYFEWYLPDDGTLWTKVANEANNLSSLGITALWLPPA |
| | | | YKGTSRSDVGYGVYDLYDLGEFNQKGAVRTKYGTKAQYL |
| | | | QAIQAAHAAGMQVYADVVFDHKGGADGTEWVDAVEVNP |
| | | | SDRNQEISGTYQIQAWTKFDFPGRGNTYSSFKWRWYHF |
| | | | DGVDWDESRKLSRIYKFRGIGKAWDWEVDTENGNYDYL |
| | | | MYADLDMDHPEVVTELKSWGKWYVNTTNIDGFRLDAVKH |
| | | | IKFSFFPDWLSDVRSQTGKPLFTVGEYWSYDINKLHNYIM |
| | | | KTNGTMSLFDAPLHNKFYTASKSGGTFDMRTLMTNTLMK |
| | | | DQPTLAVTFVDNHDTEPGQALQSWVDPWFKPLAYAFILT |
| | | | RQEGYPCVFYGDYYGIPQYNIPSLKSKIDPLLIARRDYAYG |
| | | | TQHDYLDHSDIIGWTREGVTEKPGSGLAALITDGPGGSK |
| | | | WMYVGKQHAGKVFYDLTGNRSDTVTINSDGWGEFKVNG |
| | | | GSVSVWVPRKTTVSTIAWSITTRPWTDEFVRWTEPRLVA |
| | | | WP |
| AAA22235.2 | Geobacillus | 414 | AAPFNGTMMQYFEWYLPDDGTLWTKVANEANNLSSLGIT |
| (6X-His) | stearothermophilus | | ALWLPPAYKGTSRSDVGYGVYDLYDLGEFNQKGAVRTKY |
| | | | GTKAQYLQAIQAAHAAGMQVYADVVFDHKGGADGTEWV |
| | | | DAVEVNPSDRNQEISGTYQIQAWTKFDFPGRGNTYSSFK |
| | | | WRWYHFDGVDWDESRKLSRIYKFRGIGKAWDWEVDTEN |
| | | | GNYDYLMYADLDMDHPEVVTELKSWGKWYVNTTNIDGF |
| | | | RLDAVKHIKFSFFPDWLSDVRSQTGKPLFTVGEYWSYDIN |
| | | | KLHNYIMKTNGTMSLFDAPLHNKFYTASKSGGTFDMRTL |
| | | | MTNTLMKDQPTLAVTFVDNHDTEPGQALQSWVDPWFKP |
| | | | LAYAFILTRQEGYPCVFYGDYYGIPQYNIPSLKSKIDPLLIA |
| | | | RRDYAYGTQHDYLDHSDIIGWTREGVTEKPGSGLAALITD |
| | | | GPGGSKWMYVGKQHAGKVFYDLTGNRSDTVTINSDGWG |
| | | | EFKVNGGSVSVWVPRKTTVSTIAWSITTRPWTDEFVRWT |
| | | | EPRLVAWPHHHHHH |
| AHZ45717.1 | Thermotoga | 415 | QSLVSSNPHSNSTNTGDSASNLSINEVKYPVVYEIFIRSLY |
| | petrophila (strain | | DSDGDGVGDINGVSQKVDYLRKLGIDAVWFMPFNEAVSY |
| | ATCC BAA-488/ | | HGYDITDYYNVEKDYGTMEDLENMIQVLHENGIKVIMDLVI |
| | DSM 13995/ | | NHTSDEHPWFKDAVENTTSSPYWDYYIMSLEDHSGQDH |
| | JCM 10881/ | | WHWKINSKGQKVWYFGLFGYNMPDLNHDSQKVREEVKK |
| | RKU-1) | | IVDFWISKGVDGFRIDAAKHIYGWSWDDGIQESAEYFEWF |
| | | | RDYVLSKKPDAILVGEVFSGNTYDLSLYPIPVFNFALMYSI |
| | | | RNYPEGQDGMIENNWVEESFLFLENHDLHRFFSHLQEHY |
| | | | KKFSESDYEFIKKRAALWYFLIFTLKGSPVIYYGGEIGTRGF |
| | | | KWHGPVYDEPVREPMQWYASGTGEGQTFWTKEVYKNA |
| | | | GITFGNADVDGCIYDDPYDGFSVEEQENDPKSLLNFIRFIL |
| | | | NFRKDHDAILNGDQTIFRDWKNLIAFYRESSNEKLLVVLNP |
| | | | DPVWQNSFTFEENMTMILEVDFENFIWNESNVSFSAGES |
| | | | FTVDPMKAYIFKK |
| AHZ45717.1 | Thermotoga | 416 | QSLVSSNPHSNSTNTGDSASNLSINEVKYPVVYEIFIRSLY |
| (6X-His) | petrophila (strain | | DSDGDGVGDINGVSQKVDYLRKLGIDAVWFMPFNEAVSY |
| | ATCC BAA-488/ | | HGYDITDYYNVEKDYGTMEDLENMIQVLHENGIKVIMDLVI |
| | DSM 13995/ | | NHTSDEHPWFKDAVENTTSSPYWDYYIMSLEDHSGQDH |
| | JCM 10881/ | | WHWKINSKGQKVWYFGLFGYNMPDLNHDSQKVREEVKK |
| | RKU-1) | | IVDFWISKGVDGFRIDAAKHIYGWSWDDGIQESAEYFEWF |
| | | | RDYVLSKKPDAILVGEVFSGNTYDLSLYPIPVFNFALMYSI |
| | | | RNYPEGQDGMIENNWVEESFLFLENHDLHRFFSHLQEHY |
| | | | KKFSESDYEFIKKRAALWYFLIFTLKGSPVIYYGGEIGTRGF |
| | | | KWHGPVYDEPVREPMQWYASGTGEGQTFWTKEVYKNA |
| | | | GITFGNADVDGCIYDDPYDGFSVEEQENDPKSLLNFIRFIL |
| | | | NFRKDHDAILNGDQTIFRDWKNLIAFYRESSNEKLLVVLNP |
| | | | DPVWQNSFTFEENMTMILEVDFENFIWNESNVSFSAGES |
| | | | FTVDPMKAYIFKKHHHHHH |
| PKD31675.1 | Ruminococcus | 417 | MKKILSILLAGAMLAGCVLTGCSDSSSSKSDSSDGNTAKV |
| | bromii | | ADPIEGVKATASTDKYRNYYEIFVNSFCDSNGDETGDLQG |
| | | | IISQLDYLNDGDPNSGDDLGVDAIWLTPIMPSKSYHKYDVE |
| | | | DYYNIDPDFGTLDDFDKLIEECHKRGINVILDLVLNHASSK |
| | | | NPLFTKAVEEVADNKLDGNAEYFEIHKASYFDSNTQTISLG |
| | | | NGYACEANFSQEMPEWNLNSKKTREEFTKIAKFWLDRGV |
| | | | DGFRLDACKYFTNKETDGTEFLKWFYDTCKGIKEDVYMV |
| | | | GENWTDDSDIQELYKSGIDSQFAFKFSTSTGTIISNIISQGG |
| | | | MATAKKIMNYDNKMAESNPNAINAMFLSNHDQVRSGNAL |
| | | | ESQGLSSQKLAAAVYMLAPGNPFIYYGEEIGIKAPNTTNDA |
| | | | AYRTQMVFDSENLPDIYVNGIGDEPDVPTGGGVKQQLAD |
| | | | KDSLLNYYRRIITIKNQNPEIARGRIVGTQGFDDKTVGAYY |
| | | | VEYEDSKLLIIHNFSKNDAKELTITDDMIKNATLRADLIPESS |
| | | | SKHTELKDGKISVPPQSTVIIKSAE |
| PKD31675.1 | Ruminococcus | 418 | CSDSSSSKSDSSDGNTAKVADPIEGVKATASTDKYRNYY |
| (6X-His) | bromii | | EIFVNSFCDSNGDETGDLQGIISQLDYLNDGDPNSGDDLG |
| | | | VDAIWLTPIMPSKSYHKYDVEDYYNIDPDFGTLDDFDKLIE |
| | | | ECHKRGINVILDLVLNHASSKNPLFTKAVEEVADNKLDGN |
| | | | AEYFEIHKASYFDSNTQTISLGNGYACEANFSQEMPEWNL |
| | | | NSKKTREEFTKIAKFWLDRGVDGFRLDACKYFTNKETDGT |
| | | | EFLKWFYDTCKGIKEDVYMVGENWTDDSDIQELYKSGIDS |
| | | | QFAFKFSTSTGTIISNIISQGGMATAKKIMNYDNKMAESNP |
| | | | NAINAMFLSNHDQVRSGNALESQGLSSQKLAAAVYMLAP |
| | | | GNPFIYYGEEIGIKAPNTTNDAAYRTQMVFDSENLPDIYVN |
| | | | GIGDEPDVPTGGGVKQQLADKDSLLNYYRRIITIKNQNPEI |
| | | | ARGRIVGTQGFDDKTVGAYYVEYEDSKLLIIHNFSKNDAK |
| | | | ELTITDDMIKNATLRADLIPESSSKHTELKDGKISVPPQSTV |
| | | | IIKSAEHHHHHH |
| ACD93218.3 | Bacillus sp. KR- | 419 | MFAKRLNTSFLPLFAGLLLLFHLILAGPVAVNAETANQSNE |
| | 8104 | | YTAPSIKSGTILHAWNWSFNTLKNNMKDIHDAGYTAIQTSP |
| | | | IKQVKEGNNGDKSMGNWYWLYQPTSYQIGNRYLGSEEE |
| | | | FKEMCAAAEEYGVKVIVDAVINHTTSDYAAISNEIKSISNW |
| | | | THGNTQIKNWSDRWDVTQNSLLGLYDWNTQNTQVQSYL |
| | | | KRFLERALNDGADGFRYDAAKHIELPDDGNYGSQFWPNI |
| | | | TNTSAEFQYGEILQDSASRDAAYANYMNVTASNYGHSIRS |
| | | | ALKNRNLSVSNISHYASDVSADKLVTWVESHDTYANDDE |
| | | | ESTWMSDDDIRLGWAVIASRSGSTPLFFSRPDGGGNGVR |
| | | | FPGKTQIGDRGSALFEDQAIVAVNTFHNVMAGQPEELSNP |
| | | | NGNNQIFMNQRGSKGVVLANAGSSSVSINASTKLPDGSY |
| | | | DNKAGTGSFQVRDGKLTGTINARSVAVLYPDDIANAPHVF |
| | | | LEDVKTGVTHSFNDQLTITLRADVNTAKAVYQINNGQETA |
| | | | FKDGDQLTIGKGDPFGTTYDITLTGTNSDGVTRAQEYSFV |
| | | | KRDPSAAKTIGYQNPNHWGHVNAYIYKHDGGRAIELTGS |
| | | | WPGKAMTKNADGIYTLTLPADTDTTNAKVIFNNGSAQVPG |
| | | | QNQPGFDYVQNGLYNNSGLSGSLPH |
| ACD93218.3 | Bacillus sp. KR- | 420 | ETANQSNEYTAPSIKSGTILHAWNWSFNTLKNNMKDIHDA |
| (6X-His) | 8104 | | GYTAIQTSPIKQVKEGNNGDKSMGNWYWLYQPTSYQIGN |
| | | | RYLGSEEEFKEMCAAAEEYGVKVIVDAVINHTTSDYAAISN |
| | | | EIKSISNWTHGNTQIKNWSDRWDVTQNSLLGLYDWNTQN |
| | | | TQVQSYLKRFLERALNDGADGFRYDAAKHIELPDDGNYG |
| | | | SQFWPNITNTSAEFQYGEILQDSASRDAAYANYMNVTAS |
| | | | NYGHSIRSALKNRNLSVSNISHYASDVSADKLVTWVESHD |
| | | | TYANDDEESTWMSDDDIRLGWAVIASRSGSTPLFFSRPD |
| | | | GGGNGVRFPGKTQIGDRGSALFEDQAIVAVNTFHNVMAG |
| | | | QPEELSNPNGNNQIFMNQRGSKGVVLANAGSSSVSINAS |
| | | | TKLPDGSYDNKAGTGSFQVRDGKLTGTINARSVAVLYPD |
| | | | DIANAPHVFLEDVKTGVTHSFNDQLTITLRADVNTAKAVYQ |
| | | | INNGQETAFKDGDQLTIGKGDPFGTTYDITLTGTNSDGVT |
| | | | RAQEYSFVKRDPSAAKTIGYQNPNHWGHVNAYIYKHDGG |
| | | | RAIELTGSWPGKAMTKNADGIYTLTLPADTDTTNAKVIFNN |
| | | | GSAQVPGQNQPGFDYVQNGLYNNSGLSGSLPHHHHHHH |
| AFI49455.1 | Anoxybacillus | 421 | MKRVFRALLIFALLLSVTTPVSAKTERTWQDERIYFIMVDR |
| | ayderensis | | FNNGNPKNDYEVDVHDPKAYHGGDLQGIIDKLDYIKEMGF |
| | | | TAIWLTPIFANEKGGYHGYWIEDFYKVEEHFGTLDDFKRL |
| | | | VKEAHKRDMKVILDFVVNHTGYNHPWLNDPAKKDWFHEK |
| | | | KDIFNWANQQEVENGWLFGLPDLAQENPEVKTYLFDVAK |
| | | | WWIQETDIDGYRLDTVKHVPKWFWDEFAKEVKSVKQDFF |
| | | | LLGEVWHDDPRYVAEYGKHGIDALIDFPFYKEASTIFSNVD |
| | | | QSLEPLYNVWKRNVAFYERPYLLGTFLDNHDTVRFTRLAL |
| | | | QNRINPVTRLKLGLTYLFSAPGIPIMYYGTEIALDGGEDPD |
| | | | NRRLMNFRTDKELIDYVTKLGELRAKLPSLRRGDFELLYE |
| | | | KDGMALFKRTYEKETTVIAINNTSKTQKVTLDGELEQGKEL |
| | | | RGLLAGDLVRSKDGKYDIILDRETAEIYVLAPKTGLNIPFIA |
| | | | ALVVVYTAFGLFLYFARKRKAS |
| AFI49455.1 | Anoxybacillus | 422 | KTERTWQDERIYFIMVDRFNNGNPKNDYEVDVHDPKAYH |
| (6X-His) | ayderensis | | GGDLQGIIDKLDYIKEMGFTAIWLTPIFANEKGGYHGYWIE |
| | | | DFYKVEEHFGTLDDFKRLVKEAHKRDMKVILDFVVNHTGY |
| | | | NHPWLNDPAKKDWFHEKKDIFNWANQQEVENGWLFGLP |
| | | | DLAQENPEVKTYLFDVAKWWIQETDIDGYRLDTVKHVPK |
| | | | WFWDEFAKEVKSVKQDFFLLGEVWHDDPRYVAEYGKHG |
| | | | IDALIDFPFYKEASTIFSNVDQSLEPLYNVWKRNVAFYERP |
| | | | YLLGTFLDNHDTVRFTRLALQNRINPVTRLKLGLTYLFSAP |
| | | | GIPIMYYGTEIALDGGEDPDNRRLMNFRTDKELIDYVTKLG |
| | | | ELRAKLPSLRRGDFELLYEKDGMALFKRTYEKETTVIAINN |
| | | | TSKTQKVTLDGELEQGKELRGLLAGDLVRSKDGKYDIILD |
| | | | RETAEIYVLAPKTGLNIPFIAALVVVYTAFGLFLYFARKRKA |
| | | | SHHHHHH |
| AEM05860.1 | Bacillus | 423 | ASLNGTLMQYFEWYMPNDGQHWKRLQNDSAYLAEHGIT |
| | licheniformis | | AVWIPPAYKGTSQDDVGYGAYDLYDLGEFHQKGTVRTKY |
| | | | GTKGELQSAINSLHSRDINVYGDVVINHKGGADATEYVTA |
| | | | VEVDPADRNRVTSGEQRIKAWTHFQFPGRGSTYSDFKW |
| | | | YWYHFDGTDWDESRKLNRIYKFQGKAWDWEVSNENGN |
| | | | YDYLMYADIDYDHPDVTAEIKRWGTWYANELQLDGFRLD |
| | | | AVKHIKFSFLRDWVNHVREKTGKEMFTVAEYWQNDLGAL |
| | | | ENYLNKTNFNHSVFDVPLHYQFHAASTQGGGYDMRKLLN |
| | | | GTVVSKHPVKAVTFVDNHDTQPGQSLESTVQTWFKPLAY |
| | | | AFILTREAGYPQIFYGDMYGTKGASQREIPALKHKIEPILKA |
| | | | RKQYAYGAQHDYFDHHNIVGWTREGDSSVANSGLAALIT |
| | | | DGPGGTKRMYVGRQNAGETWHDITGNRSDSVVINAEGW |
| | | | GEFHVNGGSVSIYVQR |
| AEM05860.1 | Bacillus | 424 | ASLNGTLMQYFEWYMPNDGQHWKRLQNDSAYLAEHGIT |
| (6X-His) | licheniformis | | AVWIPPAYKGTSQDDVGYGAYDLYDLGEFHQKGTVRTKY |
| | | | GTKGELQSAINSLHSRDINVYGDVVINHKGGADATEYVTA |
| | | | VEVDPADRNRVTSGEQRIKAWTHFQFPGRGSTYSDFKW |
| | | | YWYHFDGTDWDESRKLNRIYKFQGKAWDWEVSNENGN |
| | | | YDYLMYADIDYDHPDVTAEIKRWGTWYANELQLDGFRLD |
| | | | AVKHIKFSFLRDWVNHVREKTGKEMFTVAEYWQNDLGAL |
| | | | ENYLNKTNFNHSVFDVPLHYQFHAASTQGGGYDMRKLLN |
| | | | GTVVSKHPVKAVTFVDNHDTQPGQSLESTVQTWFKPLAY |
| | | | AFILTREAGYPQIFYGDMYGTKGASQREIPALKHKIEPILKA |
| | | | RKQYAYGAQHDYFDHHNIVGWTREGDSSVANSGLAALIT |
| | | | DGPGGTKRMYVGRQNAGETWHDITGNRSDSVVINAEGW |
| | | | GEFHVNGGSVSIYVQRHHHHHH |
| RU361134 | Geobacillus sp. | 425 | MKKTWWKEGVAYQIYPRSFMDANGDGIGDLRGIIEKLDYL |
| | HTA-462 | | VELGVDIVWICPIYRSPNADNGYDISDYYAIMDEFGTMDDF |
| | | | DELLAQAHRRGLKVILDLVINHTSDEHPWFIESRSSRDNPK |
| | | | RDWYIWRDGKDGREPNNWESIFGGSAWQYDERTGQYY |
| | | | LHIFDVKQPDLNWENSEVRQALYEMVNWWLDKGIDGFRI |
| | | | DAISHIKKKPGLPDLPNPKGLKYVPSFAGHMNQPGIMEYL |
| | | | RELKEQTFARYDIMTVGEANGVTVDEAEQWVGEENGVFN |
| | | | MIFQFEHLGLWERRADGSIDVRRLKRTLTKWQKGLENRG |
| | | | WNALFLENHDLPRSVSTWGNDRDYWAESAKALGALYFF |
| | | | MQGTPFIYQGQEIGMTNVRFDDIRDYRDVSALRLYELERA |
| | | | KGRTHEEAMTIIWKTGRDNSRTPMQWSGASNAGFTTGTP |
| | | | WIKVNENYRTINVEAERRDPNSVWSFYRQMIQLRKANELF |
| | | | VYGTYDLLLENHPSIYAYTRTLGRDRALVVVNLSDRPSLY |
| | | | RYDGFRLQSSDLALSNYPVRPHKNATRFKLKPYEARVYIW |
| | | | KE |
| RU361134 | Geobacillus sp. | 426 | KKTWWKEGVAYQIYPRSFMDANGDGIGDLRGIIEKLDYLV |
| (6X-His) | HTA-462 | | ELGVDIVWICPIYRSPNADNGYDISDYYAIMDEFGTMDDFD |
| | | | ELLAQAHRRGLKVILDLVINHTSDEHPWFIESRSSRDNPKR |
| | | | DWYIWRDGKDGREPNNWESIFGGSAWQYDERTGQYYL |
| | | | HIFDVKQPDLNWENSEVRQALYEMVNWWLDKGIDGFRID |
| | | | AISHIKKKPGLPDLPNPKGLKYVPSFAGHMNQPGIMEYLR |
| | | | ELKEQTFARYDIMTVGEANGVTVDEAEQWVGEENGVFN |
| | | | MIFQFEHLGLWERRADGSIDVRRLKRTLTKWQKGLENRG |
| | | | WNALFLENHDLPRSVSTWGNDRDYWAESAKALGALYFF |
| | | | MQGTPFIYQGQEIGMTNVRFDDIRDYRDVSALRLYELERA |
| | | | KGRTHEEAMTIIWKTGRDNSRTPMQWSGASNAGFTTGTP |
| | | | WIKVNENYRTINVEAERRDPNSVWSFYRQMIQLRKANELF |
| | | | VYGTYDLLLENHPSIYAYTRTLGRDRALVVVNLSDRPSLY |
| | | | RYDGFRLQSSDLALSNYPVRPHKNATRFKLKPYEARVYIW |
| | | | KEHHHHHH |
| CAB93517.1 | Geobacillus | 427 | MLTFHRIIRKGWMFLLAFLLTASLFCPTGQPAKAAAPFNGT |
| | stearothermophilus | | MMQYFEWYLPDDGTLWTKVANEANNLSSLGITALWLPPA |
| | | | YKGTSRSDVGYGVYDLYDLGEFNQKGTVRTKYGTKAQYL |
| | | | QAIQAAHAAGMQVYADVVFDHKGGADGTEWVDAVEVNP |
| | | | SDRNQEISGTYQIQAWTKFDFPGRGNTYSSFKWRWYHF |
| | | | DGVDWDESRKLSRIYKFRGIGKAWDWEVDTENGNYDYL |
| | | | MYADLDMDHPEVVTELKNWGKWYVNTTNIDGFRLDAVK |
| | | | HIKFSFFPDWLSYVRSQTGKPLFTVGEYWSYDINKLHNYIT |
| | | | KTNGTMSLFDAPLHNKFYTASKSGGAFDMRTLMTNTLMK |
| | | | DQPTLAVTFVDNHDTEPGQALQSWVDPWFKPLAYAFILT |
| | | | RQEGYPGVFYGDYYGIPQYNIPSLKSKIDPLLIARRDYAYG |
| | | | TQHDYLDHSDIIGWTREGVTEKPGSGLAALITDGPGGSK |
| | | | WMYVGKQHAGKVFYDLTGNRSDTVTITSDGWGEFKVNG |
| | | | GSVSVWVPRKTTVSTITRPITTRPWTGEFVRWTEPRLVA |
| | | | WP |
| CAB93517.1 | Geobacillus | 428 | AAPFNGTMMQYFEWYLPDDGTLWTKVANEANNLSSLGIT |
| (6X-His) | stearothermophilus | | ALWLPPAYKGTSRSDVGYGVYDLYDLGEFNQKGTVRTKY |
| | | | GTKAQYLQAIQAAHAAGMQVYADVVFDHKGGADGTEWV |
| | | | DAVEVNPSDRNQEISGTYQIQAWTKFDFPGRGNTYSSFK |
| | | | WRWYHFDGVDWDESRKLSRIYKFRGIGKAWDWEVDTEN |
| | | | GNYDYLMYADLDMDHPEVVTELKNWGKWYVNTTNIDGF |
| | | | RLDAVKHIKFSFFPDWLSYVRSQTGKPLFTVGEYWSYDIN |
| | | | KLHNYITKTNGTMSLFDAPLHNKFYTASKSGGAFDMRTLM |
| | | | TNTLMKDQPTLAVTFVDNHDTEPGQALQSWVDPWFKPLA |
| | | | YAFILTRQEGYPGVFYGDYYGIPQYNIPSLKSKIDPLLIARR |
| | | | DYAYGTQHDYLDHSDIIGWTREGVTEKPGSGLAALITDGP |
| | | | GGSKWMYVGKQHAGKVFYDLTGNRSDTVTITSDGWGEF |
| | | | KVNGGSVSVWVPRKTTVSTITRPITTRPWTGEFVRWTEP |
| | | | RLVAWPHHHHHH |
| RU361134 | Caldanaerobacter | 429 | MRKNFKAFVALFAAILLFFSGCSSKQEAKAPKSEVIYQVM |
| | subterraneus | | VDRFYNGDPSNDDPEVSKGMFDPTHTNWRMYWGGDLK |
| | subsp. | | GLTEKIPYIKGMGVTAIWISPVVDNINKLAVYNGEINAPYHG |
| | yonseiensis KB-1 | | YWARDFKRVEEHFGTWEDFDNFVKVAHENGIKVILDFAP |
| | | | NHTSPADEENPDFAENGALYDDGKLLGTYSNDSLKLFHH |
| | | | NGSISNWNNLKELQDKNLFDLADLDQSNPIVDKYLKDSIKL |
| | | | WFNHEIDGVRLDAAKHMPMEWVKSFANTIYSIKKDVLLFG |
| | | | EWMLSGPTDPLYGYNIQFANTTGFSVLDFMLNGAIRDVFG |
| | | | KGYGFERLNDTLEDTNKDYENPYKLVTFIDNHDMPRFLSL |
| | | | NNDKDKLHEAIAFIMTTRGIPVIYYGTEQYLHNDTNGGNDP |
| | | | YNRPMMEKFDESTKAYTLIKELSRLRQLTPALQYGTTTAR |
| | | | YISDDVYIYERQYGKDVVLVAINKGEKTTVKAVKTSLRKGI |
| | | | YKDYLKGLLKGVELKVTKGNGENLVQGLALPGDSVSVWT |
| | | | NVRVK |
| RU361134 | Caldanaerobacter | 430 | CSSKQEAKAPKSEVIYQVMVDRFYNGDPSNDDPEVSKG |
| (6X-His) | subterraneus | | MFDPTHTNWRMYWGGDLKGLTEKIPYIKGMGVTAIWISP |
| | subsp. | | WVDNINKLAVYNGEINAPYHGYWARDFKRVEEHFGTWED |
| | yonseiensis KB-1 | | FDNFVKVAHENGIKVILDFAPNHTSPADEENPDFAENGAL |
| | | | YDDGKLLGTYSNDSLKLFHHNGSISNWNNLKELQDKNLF |
| | | | DLADLDQSNPIVDKYLKDSIKLWFNHEIDGVRLDAAKHMP |
| | | | MEWVKSFANTIYSIKKDVLLFGEWMLSGPTDPLYGYNIQF |
| | | | ANTTGFSVLDFMLNGAIRDVFGKGYGFERLNDTLEDTNKD |
| | | | YENPYKLVTFIDNHDMPRFLSLNNDKDKLHEAIAFIMTTRG |
| | | | IPVIYYGTEQYLHNDTNGGNDPYNRPMMEKFDESTKAYTL |
| | | | IKELSRLRQLTPALQYGTTTARYISDDVYIYERQYGKDVVL |
| | | | VAINKGEKTTVKAVKTSLRKGIYKDYLKGLLKGVELKVTKG |
| | | | NGENLVQGLALPGDSVSVWTNVRVKHHHHHH |
| CBK89424.1 | [Eubacterium] | 431 | MINNKIGGRNMVGRAVNRLAGRLTAVALTAAMAVSMLAG |
| | rectale DSM | | CAAGGKNTETAAKKEYKPSAMEQMNAKNDPNVIQDNYRT |
| | 17629 | | CYEVFVYSFFDSDGDGIGDLKGLTEKLDYIEGLGCNEIWM |
| | | | MPIMPSPSYHKYDITDYMNIDKQYGTLDDFDALITECHKRN |
| | | | INVIIDFVINHTSNEHPWFKAAADYIKSLPDGAEPDSSECPY |
| | | | VDYYNFSKTNTGGYNQLPGTNWYYESQFVDSMPDLNLQ |
| | | | SEAVRGEIDKVTSFWLDRGVDGFRLDAVIYYNNNNQTETI |
| | | | DDLTWLVNNVKSKKADAYMVGEGWTTYREYAKYYKSGID |
| | | | SMFNFDFSQQDGYIGKVLNGAANHGASTYGNALVDVENE |
| | | | IKKYTDSYIDAPFYTNHDMGRSAGYYNGDNAEEKTKMAQ |
| | | | AMNLLMPGNAFLYYGEEIGMRGTANDETKRLAMRWSGD |
| | | | SKAKGMCVGPQNAEETEQTYDTLDKQMEDPYSIYNFVKQ |
| | | | TISIRNAFPEIARGTNTFEKDLSNDNVCIFTREYNGEKAVLI |
| | | | FNPSKDEASVDVSSLGVNDAVAMLQTTKKAPSYKDGTAK |
| | | | LPAYSVLVLK |
| CBK89424.1 | [Eubacterium] | 432 | CAAGGKNTETAAKKEYKPSAMEQMNAKNDPNVIQDNYRT |
| (6X-His) | rectale DSM | | CYEVFVYSFFDSDGDGIGDLKGLTEKLDYIEGLGCNEIWM |
| | 17629 | | MPIMPSPSYHKYDITDYMNIDKQYGTLDDFDALITECHKRN |
| | | | INVIIDFVINHTSNEHPWFKAAADYIKSLPDGAEPDSSECPY |
| | | | VDYYNFSKTNTGGYNQLPGTNWYYESQFVDSMPDLNLQ |
| | | | SEAVRGEIDKVTSFWLDRGVDGFRLDAVIYYNNNNQTETI |
| | | | DDLTWLVNNVKSKKADAYMVGEGWTTYREYAKYYKSGID |
| | | | SMFNFDFSQQDGYIGKVLNGAANHGASTYGNALVDVENE |
| | | | IKKYTDSYIDAPFYTNHDMGRSAGYYNGDNAEEKTKMAQ |
| | | | AMNLLMPGNAFLYYGEEIGMRGTANDETKRLAMRWSGD |
| | | | SKAKGMCVGPQNAEETEQTYDTLDKQMEDPYSIYNFVKQ |
| | | | TISIRNAFPEIARGTNTFEKDLSNDNVCIFTREYNGEKAVLI |
| | | | FNPSKDEASVDVSSLGVNDAVAMLQTTKKAPSYKDGTAK |
| | | | LPAYSVLVLKHHHHHH |
| CCC77722.1 | Lactiplantibacillus | 433 | MARDTQTQLRNEMIYSVFVRNYSEAGNFAGVTADLQRIK |
| | plantarum (strain | | DLGTDILWLLPINPIGEVNRKGTLGSPYAIKDYRGINPEYGT |
| | ATCC BAA-793/ | | LADFKALTDRAHELGMKVMLDIVYNHTSPDSVLATEHPEW |
| | NCIMB 8826/ | | FYHDADGQLTNKVGDWSDVKDLDYGHHELWQYQIDTLLY |
| | WCFS1) | | WSQFVDGYRCDVAPLVPLDFWLEARKQVNAKYPETLWL |
| | | | AESAGSGFIEELRSQGYTGLSDSELYQAFDMTYDYDVFG |
| | | | DFKDYWQGRSTVERYVDLLQRQDATFPGNYVKMRFLEN |
| | | | HDNARMMSLMHSKAEAVNNLTWIFMQRGIPLIYNGQEFL |
| | | | AEHQPSLFDRDTMVADRHGDVTPLIQKLVTIKQLPLLRAA |
| | | | DYQLAVVEEGIVKITYRAAGEALTAWIPLKGQVTAVATKLA |
| | | | AGSYQNLLTDGPTEVVDGKLTVDGQPVLIKYVTNTAVTKV |
| | | | ADQSN |
| CCC77722.1 | Lactiplantibacillus | 434 | ARDTQTQLRNEMIYSVFVRNYSEAGNFAGVTADLQRIKDL |
| (6X-His) | plantarum (strain | | GTDILWLLPINPIGEVNRKGTLGSPYAIKDYRGINPEYGTLA |
| | ATCC BAA-793/ | | DFKALTDRAHELGMKVMLDIVYNHTSPDSVLATEHPEWF |
| | NCIMB 8826/ | | YHDADGQLTNKVGDWSDVKDLDYGHHELWQYQIDTLLY |
| | WCFS1) | | WSQFVDGYRCDVAPLVPLDFWLEARKQVNAKYPETLWL |
| | | | AESAGSGFIEELRSQGYTGLSDSELYQAFDMTYDYDVFG |
| | | | DFKDYWQGRSTVERYVDLLQRQDATFPGNYVKMRFLEN |
| | | | HDNARMMSLMHSKAEAVNNLTWIFMQRGIPLIYNGQEFL |
| | | | AEHQPSLFDRDTMVADRHGDVTPLIQKLVTIKQLPLLRAA |
| | | | DYQLAVVEEGIVKITYRAAGEALTAWIPLKGQVTAVATKLA |
| | | | AGSYQNLLTDGPTEVVDGKLTVDGQPVLIKYVTNTAVTKV |
| | | | ADQSNHHHHHH |
| CAA31218.1 | Aspergillus | 435 | MMVAWWSLFLYGLQVAAPALAATPADWRSQSIYFLLTDR |
| | oryzae (strain | | FARTDGSTTATCNTADQKYCGGTWQGIIDKLDYIQGMGF |
| | ATCC 42149/ | | TAIWITPVTAQLPQTTAYGDAYHGYWQQDIYSLNENYGTA |
| | RIB 40) | | DDLKALSSALHERGMYLMVDVVANHMGYDGAGSSVDYS |
| | | | VFKPFSSQDYFHPFCFIQNYEDQTQVEDCWLGDNTVSLP |
| | | | DLDTTKDVVKNEWYDWVGSLVSNYSIDGLRIDTVKHVQK |
| | | | DFWPGYNKAAGVYCIGEVLDGDPAYTCPYQNVMDGVLN |
| | | | YPIYYPLLNAFKSTSGSMDDLYNMINTVKSDCPDSTLLGTF |
| | | | VENHDNPRFASYTNDIALAKNVAAFIILNDGIPIIYAGQEQH |
| | | | YAGGNDPANREATWLSGYPTDSELYKLIASANAIRNYAIS |
| | | | KDTGFVTYKNWPIYKDDTTIAMRKGTDGSQIVTILSNKGAS |
| | | | GDSYTLSLSGAGYTAGQQLTEVIGCTTVTVGSDGNVPVP |
| | | | MAGGLPRVLYPTEKLAGSKICSSS |
| CAA31218.1 | Aspergillus | 436 | ATPADWRSQSIYFLLTDRFARTDGSTTATCNTADQKYCG |
| (6X-His) | oryzae (strain | | GTWQGIIDKLDYIQGMGFTAIWITPVTAQLPQTTAYGDAYH |
| | ATCC 42149/ | | GYWQQDIYSLNENYGTADDLKALSSALHERGMYLMVDVV |
| | RIB 40) | | ANHMGYDGAGSSVDYSVFKPFSSQDYFHPFCFIQNYEDQ |
| | | | TQVEDCWLGDNTVSLPDLDTTKDVVKNEWYDWVGSLVS |
| | | | NYSIDGLRIDTVKHVQKDFWPGYNKAAGVYCIGEVLDGDP |
| | | | AYTCPYQNVMDGVLNYPIYYPLLNAFKSTSGSMDDLYNMI |
| | | | NTVKSDCPDSTLLGTFVENHDNPRFASYTNDIALAKNVAA |
| | | | FIILNDGIPIIYAGQEQHYAGGNDPANREATWLSGYPTDSE |
| | | | LYKLIASANAIRNYAISKDTGFVTYKNWPIYKDDTTIAMRKG |
| | | | TDGSQIVTILSNKGASGDSYTLSLSGAGYTAGQQLTEVIG |
| | | | CTTVTVGSDGNVPVPMAGGLPRVLYPTEKLAGSKICSSS |
| | | | HHHHHH |
| P56271 | Aspergillus niger | 437 | LSAASWRTQSIYFLLTDRFGRTDNSTTATCNTGNEIYCGG |
| | | | SWQGIIDHLDYIEGMGFTAIWISPITEQLPQDTADGEAYHG |
| | | | YWQQKIYDVNSNFGTADNLKSLSDALHARGMYLMVDVVP |
| | | | DHMGYAGNGNDVDYSVFDPFDSSSYFHPYCLITDWDNLT |
| | | | MVEDCWEGDTIVSLPDLDTTETAVRTIWYDWVADLVSNY |
| | | | SVDGLRIDSVLEVQPDFFPGYNKASGVYCVGEIDNGNPAS |
| | | | DCPYQKVLDGVLNYPIYWQLLYAFESSSGSISNLYNMIKS |
| | | | VASDCSDPTLLGNFIENHDNPRFAKYTSDYSQAKNVLSYI |
| | | | FLSDGIPIVYAGEEQHYAGGKVPYNREATWLSGYDTSAEL |
| | | | YTWIATTNAIRKLAIAADSAYITYANDAFYTDSNTIAMAKGT |
| | | | SGSQVITVLSNKGSSGSSYTLTLSGSGYTSGTKLIEAYTCT |
| | | | SVTVDSSGDIPVPMASGLPRVLLPASVVDSSSLCGGSGRL |
| | | | YVE |
| P56271 | Aspergillus niger | 438 | LSAASWRTQSIYFLLTDRFGRTDNSTTATCNTGNEIYCGG |
| (6X-His) | | | SWQGIIDHLDYIEGMGFTAIWISPITEQLPQDTADGEAYHG |
| | | | YWQQKIYDVNSNFGTADNLKSLSDALHARGMYLMVDVVP |
| | | | DHMGYAGNGNDVDYSVFDPFDSSSYFHPYCLITDWDNLT |
| | | | MVEDCWEGDTIVSLPDLDTTETAVRTIWYDWVADLVSNY |
| | | | SVDGLRIDSVLEVQPDFFPGYNKASGVYCVGEIDNGNPAS |
| | | | DCPYQKVLDGVLNYPIYWQLLYAFESSSGSISNLYNMIKS |
| | | | VASDCSDPTLLGNFIENHDNPRFAKYTSDYSQAKNVLSYI |
| | | | FLSDGIPIVYAGEEQHYAGGKVPYNREATWLSGYDTSAEL |
| | | | YTWIATTNAIRKLAIAADSAYITYANDAFYTDSNTIAMAKGT |
| | | | SGSQVITVLSNKGSSGSSYTLTLSGSGYTSGTKLIEAYTCT |
| | | | SVTVDSSGDIPVPMASGLPRVLLPASVVDSSSLCGGSGRL |
| | | | YVEHHHHHH |
| AFD54462.1 | Malbranchea | 439 | MVSTALFLLAAAAGSARFAKAATPDEWRSRSIYQVLTDRF |
| | cinnamomea | | ARGDGSPDAPCDTGARKYCGGNYRGLISQLDYIQGMGF |
| | | | DSVWISPITKQFEDDWNGAPYHGYWQTDLYALNEHFGTE |
| | | | EDLRALADELHARGMFLMVDVVINHNGWPGDAASIDYSQ |
| | | | FNPFNSSDYYHPPCEINYDDQTSVEQCWLYTGANALPDL |
| | | | KTEDPHVSQVHNDWIADLVSKYSIDGLRIDTTKHVDKPAIG |
| | | | SFNDAAGVYAVGEVYHGDPAYTCPYQDWVDGVLNFPVY |
| | | | YPLIDAFKSPSGTMWSLVDNINKVFQTCNDPRLLGTFSEN |
| | | | HDIPRFASYTQDLALAKNVLAFTILFDGIPIVYAGQEQQYS |
| | | | GDSDPYNREALWLSGFNTDAPLYKHIAACNRIRSHAVSND |
| | | | DAYITTPTDIKYSDDHTLALVKGAVTTVLTNAGANAGETTV |
| | | | TVEATGYASGEQVTDVLSCESIAASDGGRLSVTLNQGLPR |
| | | | VFFPTDALAGSGLCEN |
| AGJ520811 | Rhizomucor | 440 | MKFSISLSAAIVLFAAATSLASPLPQQQRYAKRATSDDWK |
| | pusillus | | SKAIYQLLTDRFGRADDSTSNCSNLSNYCGGTYEGITKHL |
| | | | DYISGMGFDAIWISPIPKNSDGGYHGYWATDFYQLNSNFG |
| | | | DESQLKTLIQAAHERGMYVMLDVVANHAGPTSNGYSGYT |
| | | | FGDASLYHPKCTIDYNDQTSIEQCWVADELPDIDTENSDN |
| | | | VAILNDIVSGWVGNYSFDGIRIDTVKHIRKDFWTGYAEAAG |
| | | | VFATGEVFNGDPAYVGPYQKYLPPLINYPMYYALNDVFVS |
| | | | KSKGFSRISEMLGSNRNAFEDTSVLTTFVDNHDNPRFLNS |
| | | | QSDKALFKNALTYVLLGEGIPIVYYGSEQGFSGGADPANR |
| | | | EVLWTANYDTSSDLYQFIKTVNSVRMKSNKTVYMDIYVGD |
| | | | NAYAFKHGDALVVLNNYGSGSTNQVSFSVSGKFDSGASL |
| | | | MDIVSNITTTVSSDGTVTFNLKDGLPAIFTSA |
| AGJ52081.1 | Rhizomucor | 441 | ATSDDWKSKAIYQLLTDRFGRADDSTSNCSNLSNYCGGT |
| (6X-His) | pusillus | | YEGITKHLDYISGMGFDAIWISPIPKNSDGGYHGYWATDF |
| | | | YQLNSNFGDESQLKTLIQAAHERGMYVMLDVVANHAGPT |
| | | | SNGYSGYTFGDASLYHPKCTIDYNDQTSIEQCWVADELP |
| | | | DIDTENSDNVAILNDIVSGWVGNYSFDGIRIDTVKHIRKDF |
| | | | WTGYAEAAGVFATGEVFNGDPAYVGPYQKYLPPLINYPM |
| | | | YYALNDVFVSKSKGFSRISEMLGSNRNAFEDTSVLTTFVD |
| | | | NHDNPRFLNSQSDKALFKNALTYVLLGEGIPIVYYGSEQG |
| | | | FSGGADPANREVLWTANYDTSSDLYQFIKTVNSVRMKSN |
| | | | KTVYMDIYVGDNAYAFKHGDALVVLNNYGSGSTNQVSFS |
| | | | VSGKFDSGASLMDIVSNITTTVSSDGTVTFNLKDGLPAIFT |
| | | | SAHHHHHH |
| A0A5S8W | Thamnidium | 442 | AAAADWKSRSIYQLVTDRFGRSDGSTSACGDLSNYCGG |
| FA7 | elegans | | DYKGIQNQLDYIAGMGFDAIWISPIPENTDGGYHGYWAKD |
| | | | FEKLNTNFGSADDLKALVTAAHGKGMYVMLDVVANHAGP |
| | | | ASGGDYSGFTFSSASNYHPQCTIDYDNQTSVEQCWVAD |
| | | | DLPDINTEDDTIVSKLHSIVSDWVTTYDFDGIRIDTVKHIRK |
| | | | DFWSGYEEAAGVFATGEVFDGDAAYVGPYQDQLSSLINY |
| | | | PLYYAIRDVFSAGSGFSRISDMLSTIKSNFKDPSVLTTFVD |
| | | | NQDNARFLSVKSDMSLYKNALAFTILTEGIPVVYYGTEQG |
| | | | FKGGDDPKNREVLWTSNYDTSSDLYKFIKIVNNDVRQKSD |
| | | | KTVTLDVDVGTNTYAFTHGKNLIVVNNYGSGSTESVTVKV |
| | | | GDSVADGTKLVDAVSNITATVSGGSITFSLKDGLPALFVPS |
| | | | S |
| A0A5S8W | Thamnidium | 443 | AAAADWKSRSIYQLVTDRFGRSDGSTSACGDLSNYCGG |
| FA7 (6X- | elegans | | DYKGIQNQLDYIAGMGFDAIWISPIPENTDGGYHGYWAKD |
| His) | | | FEKLNTNFGSADDLKALVTAAHGKGMYVMLDVVANHAGP |
| | | | ASGGDYSGFTFSSASNYHPQCTIDYDNQTSVEQCWVAD |
| | | | DLPDINTEDDTIVSKLHSIVSDWVTTYDFDGIRIDTVKHIRK |
| | | | DFWSGYEEAAGVFATGEVFDGDAAYVGPYQDQLSSLINY |
| | | | PLYYAIRDVFSAGSGFSRISDMLSTIKSNFKDPSVLTTFVD |
| | | | NQDNARFLSVKSDMSLYKNALAFTILTEGIPVVYYGTEQG |
| | | | FKGGDDPKNREVLWTSNYDTSSDLYKFIKIVNNDVRQKSD |
| | | | KTVTLDVDVGTNTYAFTHGKNLIVVNNYGSGSTESVTVKV |
| | | | GDSVADGTKLVDAVSNITATVSGGSITFSLKDGLPALFVPS |
| | | | SHHHHHH |
| A0A5S8W | Cordyceps | 444 | MKLTASLTLLTQALAVLGADTNSWKSRTIYFALTDRVARS |
| FA8 | farinosa | | ASDNGGDGCGQLQDYCGGTFKGLEGKLDYIKGMGFDAI |
| | | | WITPWVQNSARGYHGYWASNLYATNSHYGTSDELKGLVN |
| | | | AAHGKGIYIMVDVVANHVGNGPLNEMQPAPLNQGSSYHP |
| | | | ACGINYNDQHSIETCRVASDLPDLDTTDPKIRTLYKDWIKW |
| | | | LMSTYKFDGVRIDTVKHVEKDFWPDFAWASGSYTIGEVF |
| | | | SGDPNYVAGYSKLMGGLLNYPVYFPLNRFYQQQNSSQAL |
| | | | VDMHNQIGSLVPDPTTLGTFLDNHDNPRFLSQKNDVSLFK |
| | | | NALTYVLLARGIPIVYYGSEQAYAGGGDPQNREDLWRSR |
| | | | FNTNSDMYKFFQALGGVRKSHGGLPGNDHVHLFVESDA |
| | | | YAWSRQDGAVMALTSNIGKGQQRQFCFFTQKNNKTWRG |
| | | | IFDGKTYTSGGDGKLCATVNNGEPIVFVAQ |
| A0A5S8W | Cordyceps | 445 | ADTNSWKSRTIYFALTDRVARSASDNGGDGCGQLQDYC |
| FA8 (6X- | farinosa | | GGTFKGLEGKLDYIKGMGFDAIWITPVVQNSARGYHGYW |
| His) | | | ASNLYATNSHYGTSDELKGLVNAAHGKGIYIMVDVVANHV |
| | | | GNGPLNEMQPAPLNQGSSYHPACGINYNDQHSIETCRVA |
| | | | SDLPDLDTTDPKIRTLYKDWIKWLMSTYKFDGVRIDTVKH |
| | | | VEKDFWPDFAWASGSYTIGEVFSGDPNYVAGYSKLMGG |
| | | | LLNYPVYFPLNRFYQQQNSSQALVDMHNQIGSLVPDPTTL |
| | | | GTFLDNHDNPRFLSQKNDVSLFKNALTYVLLARGIPIVYYG |
| | | | SEQAYAGGGDPQNREDLWRSRFNTNSDMYKFFQALGGV |
| | | | RKSHGGLPGNDHVHLFVESDAYAWSRQDGAVMALTSNI |
| | | | GKGQQRQFCFFTQKNNKTWRGIFDGKTYTSGGDGKLCA |
| | | | TVNNGEPIVFVAQHHHHHH |

One of skill in the art would appreciate that the lytic proteins described herein may be recombinantly produced. Accordingly, the protein may contain a suitable purification tag, such as a His tag containing, e.g., three, four, five, six, seven, eight, nine, ten, or more histidine residues present at the N-terminus or C-terminus of the protein. The protein may also contain a removable signal sequence present at the N-terminus or C-terminus of the protein.

One of skill in the art would also appreciate that the lytic proteins described herein may include biologically active fragments thereof, e.g., fragments of a lytic protein as described herein that may be truncated, e.g., by 1, 2, 3, 4, 5, 6, 7, 8, 9, 10, 15, 20, 25, 30, 35, 40, 45, 50, 60, 70, 80, 90, 100, or more amino acids but still substantially retain their biological activity.

In some embodiments, the composition includes a concentration of proteins (e.g., Lysin A, Lysin B, isoamylase, and/or α-amylase) of from 0.1 mg/mL to 20 mg/ml (e.g., e.g., from 0.1 mg/mL to 1 mg/mL, e.g., 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, or 1 mg/mL, e.g., from 1 mg/mL to 10 mg/mL, e.g., 2 mg/ml, 3 mg/mL, 4 mg/mL, 5 mg/mL, 6 mg/mL, 7 mg/mL, 8 mg/mL, 9 mg/mL, 10 mg/mL, e.g., from 10 mg/mL to 20 mg/mL, e.g., 11 mg/mL, 12 mg/mL, 13 mg/mL, 14 mg/mL, 15 mg/mL, 16 mg/mL, 17 mg/mL, 18 mg/mL, 19 mg/mL, or 20 mg/mL). In some embodiments, the composition includes a concentration of Lysin A, Lysin B, isoamylase, and/or α-amylase of from 1 mg/mL to 10 mg/mL.

### Intracellular Bacteria

Intracellular bacteria reside within a host cell where they reproduce and cause infection. Intracellular bacteria may reside within immune cells, such as professional antigen cells. Professional antigen presenting cells (APCs) include macrophages, dendritic cells, and phagocytic cells, such as macrophages. APCs process and display antigens complexed with major histocompatibility complexes (MHCs) on their surfaces. APCs put antigens up on MHC class 2 for bacterial pathogens, which are recognized by T cells, which then go onto stimulate B cells that have an antibody that is complementary to the antigen. This leads to proliferation of the specific B cells that encode for the specific antibody to combat organisms that have that antigen. Certain bacteria evade this immune response by hiding within the immune cell.

The compositions and methods described herein may be used to target a mycobacterium, such as an intracellular mycobacterium that resides in a professional antigen presenting cell (e.g., macrophage or dendritic cell). In some embodiments, the mycobacterial species is M. tuberculosis, M. leprae, M. lepromatosis, M. avium, M. kansasii, M. fortuitum, M. chelonae, M. marinum, M. intracellulare, M. abscessus, M. chimera, M. boletti, M. fortuitum, M. goodii, or M. masiliense. In particular embodiments, the mycobacterium is an NTM. In some embodiments, the NTM is M. abscessus, M. intracellulare, M. avium, M. chimera, M. boletti, M. fortuitum, M. goodii, and M. masiliense.

In some embodiments, the compositions and methods described herein may be used to target other actinomycetia (e.g., corynebacteriales or propionibacteriales) that have similar envelope components as mycobacteria. For example, the compositions and methods may be used to target a Nocardia, Corynebacterium, or Rhodococcus species. For example, the Nocardia species may be, e.g., N. brasiliensis, N. cyriacigeorgica, N. farcinica, N. nova, N. asteroids, N. brasiliensis, and N. caviae. The Corynebacterium species may be, e.g., C. glutamicum or C. diphtheriae. The Rhodococcus species may be, e.g., R. fascians or R. equi. The composition and methods may be used to target a propionibacteriales, such as a Cutibacterium species. The Cutibacterium species may be, e.g., C. acnes.

### Supramolecular Structures

Supramolecular structures may be used to formulate a cocktail of lytic enzymes for delivery. Supramolecular structures include a defined complex of molecules, e.g., lipids, held together by non-covalent bonds, such as hydrogen bonds, van der Waals forces, electrostatic interactions, ion-dipole forces, hydrophobic effect, and pi-pi interactions. Supramolecular structures may include large complexes of molecules that form sphere-, rod-, helix-, or sheet-like structures. Supramolecular structures include, for example, lipid-based supramolecular structures, such as micelles, liposomes, and LNPs. Supramolecular structures may have a pre-determined size. The size of the structure may vary based on the components, e.g., size of protein, packed within the structure. The supramolecular complex is endocytosed by a cell, e.g., a professional antigen presenting cell such as a macrophage or dendritic cell, and the antibacterial lytic proteins are delivered to the targeted intracellular compartment (endosome, phagosome, lysosome, or cytosol) wherein the bacteria reside.

In some embodiments, a particular particle size is used to access a certain endocytic route to direct the structure to the appropriate targeted intracellular compartment. The supramolecular structure may be endocytosed and delivered to the targeted intracellular compartment, e.g., via clathrin-dependent endocytosis or via caveolin-dependent endocytosis. The particle size, e.g., Z-average mean particle diameter, of the supramolecular structure may vary from 75 nm to 5 μm, e.g., from 75 nm to 2 μm, from 75 nm to 1 μm, e.g., from 75 nm to 750 nm (e.g., from 250 nm to 750 nm, or from 75 nm to 250 nm). In some embodiments, when the supramolecular structure is an LNP or micelle, the Z-average mean particle diameter is from 75 nm to 250 nm. In some embodiments, when the supramolecular structure is a vesicle (e.g., a liposome), the Z-average mean particle diameter is from 250 nm to 750 nm. Non-limiting examples of the Z-average mean particle diameters include, e.g., from 75 nm to 100 nm, e.g., from 75 nm to 85 nm, e.g., 80 nm, e.g., from 80 nm to 140 nm, from 90 nm to 130 nm, or from 110 nm to 130 nm, e.g., 120 nm, e.g., from 200 nm to 300 nm, e.g., from 250 nm to 300 nm, from 260 nm to 290 nm, from 260 nm to 280 nm, from 265 nm to 275 nm, e.g., 270 nm, e.g., from 300 nm to 400 nm, from 400 nm to 600 nm, e.g., from 450 nm to 550 nm, from 475 nm to 525 nm, from 480 nm to 520 nm, from 490 nm to 510 nm, from 495 nm to 505 nm, e.g., 500 nm, e.g., 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485 nm, 490 nm, 495 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575 nm, 580 nm, 585 nm, 590 nm, 595 nm, 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675 nm, 680 nm, 685 nm, 690 nm, 695 nm, 700 nm, 705 nm, 710 nm, 715 nm, 720 nm, 725 nm, 730 nm, 735 nm, 740 nm, 745 nm, 750 nm, 755 nm, 760 nm, 765 nm, 770 nm, 775 nm, 780 nm, 785 nm, 790 nm, 795 nm, 800 nm, 805 nm, 810 nm, 815 nm, 820 nm, 825 nm, 830 nm, 835 nm, 840 nm, 845 nm, 850 nm, 855 nm, 860 nm, 865 nm, 870 nm, 875 nm, 880 nm, 885 nm, 890 nm, 895 nm, 900 nm, 905 nm, 910 nm, 915 nm, 920 nm, 925 nm, 930 nm, 935 nm, 940 nm, 945 nm, 950 nm, 955 nm, 960 nm, 965 nm, 970 nm, 975 nm, 980 nm, 985 nm, 990 nm, 995 nm, 1 μm, 1.1 μm, 1.2 μm, 1.3 μm, 1.4 μm, 1.5 μm, 1.6 μm, 1.7 μm, 1.8 μm, 1.9 μm, 2 μm, 2.1 μm, 2.2 μm 2.3 μm, 2.4 μm, 2.5 μm, 2.6 μm, 2.7 μm, 2.8 μm, 2.9 μm, 3 μm, 3.1 μm, 3.2 μm, 3.3 μm, 3.4 μm, 3.5 μm, 3.6 μm, 3.7 μm, 3.8 μm, 3.9 μm, 4 μm, 4.1 μm 4.2 μm 4.3 μm, 4.4 μm, 4.5 μm, 4.6 μm, 4.7 μm, 4.8 μm, 4.9 μm, or 5 μm. In particular embodiments, the Z-average mean particle diameter of the supramolecular structure may be from 75 nm to 250 nm. In some embodiments, the Z-average mean particle diameter of the supramolecular structure is 80 nm, 270 nm, or 500 nm.

The mean particle diameter may be measured by zeta potential, dynamic light scattering (DLS), electrophoretic light scattering (ELS), static light scattering (SLS), molecular weight, electrophoretic mobility, size exclusion chromatography (SEC), field flow fractionation, or other methods known in the art. In particular embodiments, the mean particle diameter is measured by. In particular embodiments, the supramolecular structure contains a Z-average mean particle diameter of from 75 nm to 250 nm. In particular embodiments, the supramolecular structure contains a Z-average mean particle diameter of from 250 nm to 750 nm. In particular embodiments, the supramolecular structure contains a Z-average mean particle diameter of 500 nm. In particular embodiments, the supramolecular structure contains a Z-average mean particle diameter of 270 nm. In particular embodiments, the supramolecular structure contains a Z-average mean particle diameter of 80 nm. One of skill in the art would appreciate that a population of supramolecular structures (e.g., liposomes, LNPs, or micelles) may have a range of Z-average mean particle diameters within the population. Thus, the population may be polydisperse. The population may have a polydispersity index of 0.5 or less, e.g., 0.3 or less (e.g., 0.05 to 0.3). The polydispersity index can be determined using DLS (see, e.g., ISO 22412:2017).

The supramolecular structures may be loaded with a predetermined number of antibacterial lytic proteins or average number of antibacterial lytic proteins per supramolecular structure. For example, the supramolecular structure may contain from one protein to 10⁶ proteins (e.g., 1 to 10⁵, 1 to 10⁴, 1 to 10³, 1 to 10², 1 to 10, 10 to 10⁶, 10 to 10⁵, 10 to 10⁴, 10 to 10³, 10 to 10², 103 to 10⁶, 103 to 10⁵, 10³ to 10⁴). The number of proteins per structure may depend on the size of the protein and the size of the structure.

The supramolecular structures may include an endosomal escape moiety. Supramolecular structures including an endosomal escape moiety may provide for an improved cytosolic delivery of the cargo (e.g., a therapeutic agent) included in the supramolecular structure. Endosomal escape moieties are known in the art. In some embodiments, an endosomal escape moiety is an ionizable lipid. The ionizable lipids may also serve as supramolecular structure-layer forming lipids. Non-limiting examples of ionizable lipids include those described in, e.g., WO 2019/067875; WO 2018/191750; and U.S. Pat. No. 9,999,671. Other exemplary endosomal escape moieties include fusogenic lipids (e.g., dioleoylphosphatidyl-ethanolamine (DOPE)); and polymers such as polyethylenimine (PEI); poly (beta-amino ester) s; polypeptides, such as polyarginines (e.g., octaarginine) and polylysines (e.g., octalysine); proton sponges, viral capsids, and peptide transduction domains as described herein. For example, fusogenic peptides can be derived from the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion protein of the Sendai virus; the transmembrane glycoprotein of the Semliki forest virus; the fusion protein of the human respiratory syncytial virus (RSV); the fusion protein of the measles virus; the fusion protein of the Newcastle disease virus; the fusion protein of the visna virus; the fusion protein of murine leukemia virus; the fusion protein of the HTL virus; and the fusion protein of the simian immunodeficiency virus (SIV). Other moieties that can be employed to facilitate endosomal escape are described in Dominska et al., Journal of Cell Science, 123 (8): 1183-1189, 2010. Specific examples of endosomal escape moieties including moieties suitable for inclusion in, or conjugation to, to the supramolecular structures disclosed herein are provided, e.g., in WO 2015/188197; the disclosure of these endosomal escape moieties is incorporated by reference herein.

### Liposomes

Liposomes are useful for the transfer and delivery of antibacterial proteins to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the antibacterial protein are delivered into the cell where the antibacterial protein can specifically target and lyse a bacterial cell (e.g., mycobacterial cell, e.g., NTM cell) residing inside a mammalian immune cell. In some cases, the liposomes are also specifically targeted, e.g., to direct the protein to particular mammalian immune cell types and/or to particular intracellular compartments that typically harbor bacteria (e.g., mycobacteria) during infection (endosome, phagosome, lysosome, or cytosol). The composition of the liposome is usually a combination of phospholipids, usually in combination with steroids, such as cholesterol. Other phospholipids or other lipids may also be used. The physical characteristics of liposomes depend on pH, ionic strength, and the presence of divalent cations.

In some embodiments, a liposome described herein includes a phospholipid. In some embodiments, a glycerophospholipid, e.g., a phosphatidylserine. A phosphatidylserine is a glycerol molecule having two hydroxyl groups substituted with fatty acid ester moieties and one hydroxyl group substituted with a phosphodiester moiety that is covalently bonded to serine side chain. A typical structure of a phosphatidylserine is RO—CH₂—CH (OR)—CH₂—OP (O) (OH)—OCH₂CH (COOH) NH₂, or a salt thereof, where each R is independently a fatty acid acyl. Additionally, or alternatively, a liposome described herein may include, e.g., a lysophospholipid, e.g., a lysophosphatidylserine. A lysophosphatidylserine is a phosphatidylserine missing one of its two fatty acid ester moieties. A typical structure of a lysophosphatidylserine is RO—CH₂—CH (OR)—CH₂—OP (O) (OH)—OCH₂CH (COOH) NH₂, or a salt thereof, where one R is a fatty acid acyl, and the other R is H. Thus, in certain preferred embodiments, a liposome described herein includes RO—CH₂—CH (OR)—CH₂—OP (O) (OH)—OCH₂CH (COOH) NH₂, or a salt thereof, where each R is H or a fatty acid acyl, provided that at least one R is a fatty acid acyl.

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Cationic liposomes possess the advantage of being able to fuse to the cell membrane. Non-limiting examples of cationic lipids include N, N-dioleyl-N, N-dimethylammonium chloride (DODAC), N, N-distearyl-N, N-dimethylammonium bromide (DDAB), N-(I-(2,3-dioleoyloxy) propyl)-N,N,N-trimethylammonium chloride (DOTAP), N-(I-(2,3-dioleyloxy) propyl)-N,N,N-trimethylammonium chloride (DOTMA), N,N-dimethyl-2,3-dioleyloxy) propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), 1,2-Dilinoleoyl-3-trimethylaminopropane chloride salt (DLin-TAP.CI), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino) ethoxypropane (DLin-EG-DMA), 1,2-Dilinolenyloxy-N, N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di ((9Z,12Z)-octadeca-9,12-dienyetetrahydro-3aH-cyclopenta [d][1,3]dioxol-5-amine (ALN100), (6Z,9Z,28Z,31Z)-heptatriaconta-6,9,28,31-tetraen-19-yl4-(dimethylamino) butanoate (MC3), 1,1′-(2-(4-(2-((2-(bis(2-hydroxydodecyl) amino) ethyl) (2-hydroxydodecyl) amino)ethyl) piperazin-1-yeethylazanediyedidodecan-2-ol (Tech G1), or a mixture thereof. The cationic lipid can include, for example, from 20 mol % to 50 mol % or 40 mol % of the total lipid present in the particle.

Non-cationic liposomes, although not able to fuse as efficiently with the plasma membrane, are taken up by macrophages in vivo and can be used to deliver antibacterial proteins to macrophages. Anionic liposome compositions generally are formed from dimyristoyl phosphatidylglycerol, while anionic fusogenic liposomes are formed primarily from dioleoyl phosphatidylethanolamine (DOPE). The lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, 1,2-dioleoyl-sn-glycero-3-phospho-L-serine (sodium salt, DOPS), or a mixture thereof. The non-cationic lipid can be, for example, from 5 mol % to 90 mol %, 10 mol %, or 58 mol % if cholesterol is included, of the total lipid present in the particle. In some embodiments, a lipid can be a combination of lipids described above, e.g., a combination of lipids including DOPC, DOPS, Chol, and DOPE.

In some embodiments, the liposome includes a mixture of lipids. For example, the mixture of lipids may include two or more of DOPC, DOPE, DOPS, and cholesterol.

In some embodiments, the DOPC and DOPE are present at a molar ration of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPC and DOPS are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPC and cholesterol are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPE and DOPS are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPE and cholesterol are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPS and cholesterol are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPC, DOPE, DOPS, and cholesterol are present at a molar ratio of from 1-20:1-20:1-5:1-5. For example, in some embodiments, the DOPC, DOPE, DOPS, and cholesterol are present at a molar ratio of 10:10:3:4.

In some embodiments, the liposome includes a concentration of lipids of from 0.03 mg/mL to 10 mg/mL, e.g., 0.1 mg/mL to 10 mg/mL (e.g., from 0.1 mg/mL to 1 mg/mL, e.g., 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, or 1 mg/mL, e.g., from 1 mg/mL to 10 mg/mL, e.g., 2 mg/ml, 3 mg/mL, 4 mg/mL, 5 mg/mL, 6 mg/mL, 7 mg/mL, 8 mg/mL, 9 mg/mL, or 10 mg/mL). In some embodiments, the liposome includes a concentration of lipids of from 1 mg/ml to 5 mg/mL.

Another type of liposomal composition is formed from phosphatidylcholine (PC) such as, for example, soybean PC, and egg PC. Another type is formed from mixtures of phospholipid and/or phosphatidylcholine and/or cholesterol. Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Pub. No. 20060058255, the linking groups of which are herein incorporated by reference.

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selective for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid; peptidases (which can be substrate specific); and phosphatases.

A cleavable linkage group, such as a disulfide bond can be susceptible to pH. The pH of human serum is 7.4, while the average intracellular pH is slightly lower, ranging from 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissues. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate linkers are cleaved at least 2, 4, 10, 20, 30, 40, 50, 60, 70, 80, 90, or 100 times faster in the cell (or under in vitro conditions selected to mimic intracellular conditions) as compared to blood or serum (or under in vitro conditions selected to mimic extracellular conditions).

### Lipid Nanoparticles

Anti-bacterial agents of in the invention may be fully encapsulated in a lipid formulation, e.g., a lipid nanoparticle (LNP). LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 2000/003683. The particles of the present invention typically have a mean diameter of 50 nm to 150 nm, more typically 60 nm to 130 nm, more typically 70 nm to 110 nm, most typically 70 nm to 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

In one embodiment, the lipid to drug ratio (mass/mass ratio) (e.g., lipid to peptide ratio) will be in the range of from 1:1 to 50:1, from 1:1 to 25:1, from 3:1 to 15:1, from 4:1 to 10:1, from 5:1 to 9:1, or 6:1 to 9:1. Ranges intermediate to the above recited ranges are also contemplated to be part of the invention.

Non-limiting examples of cationic lipids include DODAC, DDAB, DOTAP, DOTMA, DODMA, DLinDMA, DLenDMA, DLin-C-DAP, DLin-DAC, DLin-MA, DLinDAP, DLin-S-DMA, DLin-2-DMAP, DLin-TMA.CI, DLin-TAP.CI, 1DLin-MPZ, DLinAP, DOAP, DLin-EG-DMA, (DLin-K-DMA or analogs thereof, ALN100, MC3, Tech G1, or a mixture thereof. The cationic lipid can include, for example, from 20 mol % to 50 mol % or 40 mol % of the total lipid present in the particle.

The lipid can be an anionic lipid or a neutral lipid including, but not limited to, DSPC, DOPC, DOPS, DPPC, DOPG, DPPG, DOPE, POPC, POPE, DOPE-mal, DPPE, DMPE, DSPE, 16-O-monomethyl PE, 16-O-dimethyl PE, 18-1-trans PE, SOPE, cholesterol, or a mixture thereof. The non-cationic lipid can be, for example, from 5 mol % to 90 mol %, 10 mol %, or 60 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C12), a PEG-dimyristyloxypropyl (C14), a PEG-dipalmityloxypropyl (C16), or a PEG-distearyloxypropyl (C18). The conjugated lipid that prevents aggregation of particles can be, for example, from 0 mol % to 20 mol % or 2 mol % of the total lipid present in the particle.

In some embodiments, the LNP further includes cholesterol at, e.g., 10 mol % to 60 mol % or 50 mol % of the total lipid present in the particle.

In some embodiments, the LNP includes a mixture of lipids. For example, the mixture of lipids may include two or more of DOPC, DOPE, DOPS, and cholesterol.

In some embodiments, the DOPC and DOPE are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPC and DOPS are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPC and cholesterol are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPE and DOPS are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPE and cholesterol are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPS and cholesterol are present at a molar ratio of from 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10).

In some embodiments, the DOPC, DOPE, DOPS, and cholesterol are present at a molar ratio of from 1-20:1-20:1-5:1-5. For example, in some embodiments, the DOPC, DOPE, DOPS, and cholesterol are present at a molar ratio of 10:10:3:4.

In some embodiments, the LNP includes a concentration of lipids of from 0.03 mg/ml to 10 mg/mL, e.g., 0.1 mg/mL to 10 mg/ml (e.g., from 0.1 mg/mL to 1 mg/mL, e.g., 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, or 1 mg/mL, e.g., from 1 mg/mL to 10 mg/mL, e.g., 2 mg/ml, 3 mg/mL, 4 mg/mL, 5 mg/mL, 6 mg/mL, 7 mg/mL, 8 mg/mL, 9 mg/mL, or 10 mg/mL). In some embodiments, the LNP includes a concentration of lipids of from 1 mg/mL to 5 mg/mL.

### Micelles

Micelles are a particular type of molecular assembly in which amphipathic molecules are arranged in a spherical structure such that all the hydrophobic portions of the molecules are directed inward, leaving the hydrophilic portions in contact with the surrounding aqueous phase. Micelles may be made of lipids. The micelle phase is caused by the packing behavior of single-tail lipids in a bilayer. The difficulty filling all the volume of the interior of a bilayer, while accommodating the area per head group forced on the molecule by the hydration of the lipid head group, leads to the formation of the micelle. This type of micelle is known as a normal-phase micelle (oil-in-water micelle). Inverse micelles have the head groups at the center with the tails extending out (water-in-oil micelle).

Micelles are approximately spherical in shape. Other phases, including shapes such as ellipsoids, cylinders, and bilayers, are also possible. The shape and size of a micelle are a function of the molecular geometry of its surfactant molecules and solution conditions such as surfactant concentration, temperature, pH, and ionic strength. The process of forming micelles is known as micellization and forms part of the phase behavior of many lipids according to their polymorphism.

### Targeting Moieties

A supramolecular structure described herein may include, e.g., a targeting moiety. A targeting moiety may be used to direct the supramolecular structure to a particular cell-type (e.g., a professional antigen-presenting cell, e.g., macrophage or dendritic cell). Certain lipids (e.g., phosphatidyl serine) may be used in the supramolecular structure (e.g., a vesicle) both as a supramolecular structure layer-forming lipid and as a targeting moiety. The targeting moiety may be, e.g., an antibody or an antigen-binding fragment or an engineered derivative thereof (e.g., Fcab or a fusion protein (e.g., scFv)). The targeting moiety may be, e.g., a polypeptide. Alternatively, the targeting moiety may be, e.g., a small molecule (e.g., mannose or folate) or a cluster of small molecules (e.g., a cluster of mannoses). A targeting moiety may be associated with a supramolecular structure covalently or non-covalently.

### Small Molecules

The targeting moiety may be a small molecule capable of complexing a receptor expressed on the surface of the targeted cell. Non-limiting examples of small molecules that may be used as targeting moieties in the supramolecular structures described herein are phosphatidylserine, lysophosphatidylserine folate, mannose, and mannose clusters.

In some embodiments, the targeting moiety is phosphatidylserine or lysophosphatidylserine. In some embodiments, the targeting moiety is phosphatidylserine. Phosphatidylserine and/or lysophosphatidylserine may be present as a supramolecular structure layer-forming lipid that is non-covalently bonded to the rest of the supramolecular structure.

Folate may be used as a targeting moiety. In the supramolecular structures described herein, folate may be of the following structure:

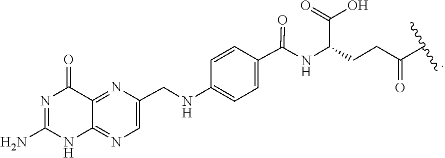 [SMILES: *C(=O)CC[C@H](NC(=O)c1ccc(NCc2cnc3[nH]c(N)nc(=O)c3n2)cc1)C(=O)O]

Mannose or a mannose cluster can be used to target the supramolecular structure described herein to dendritic cells and macrophages. Mannose clusters are known in the art.

Folate, mannose, and mannose clusters may be covalently linked to the supramolecular structure. Conjugation techniques for linking folate, mannose, and mannose clusters are known in the art, for example, as described in US 2014/0045919, U.S. Pat. Nos. 9,725,479, 8,758,810, 8,450,467, 6,525,031, 6,335,434, and 5,759,572.

### Antigen-Binding Moieties

An antigen-binding moiety in the supramolecular structure described herein can be an antibody or an antigen-binding fragment thereof, e.g., F (ab) 2 or Fab, or an engineered derivative thereof, e.g., Fcab or a fusion protein, e.g., scFv. A human or chimeric, e.g., humanized, antibody can be used as an antibody in the supramolecular structure described herein.

The antigen-binding moiety targets APCs having the surface antigen that is recognized by the antigen-binding moiety. Dendritic cells may be targeted by anti-DEC205, anti-CD304, anti-CD303, anti-CD40, anti-CD74, anti-BDCA2, or anti-CD123 antibodies or antigen-binding fragments thereof or engineered derivatives thereof. Macrophages can be targeted by anti-CD163, anti-CD40, anti-CD74, anti-CD206, or anti-CD123 antibodies or antigen-binding fragments thereof or engineered derivatives thereof.

Non-limiting examples of anti-CD38 antibodies are daratumumab, SAR650984, MOR202, or any one of antibodies Ab79, Ab19, Ab43, Ab72, and Ab110 disclosed in WO 2012/092616, the disclosure of these antibodies is incorporated herein by reference. A non-limiting example of an anti-CD79b antibody is huMA79b v28 disclosed in WO 2014/011521. A non-limiting example of an anti-CD22 antibody is 10F4 disclosed in US 2014/0127197. A non-limiting example of an anti-CD20 antibody is rituximab. A non-limiting example of an anti-DEC205 antibody is provided in US 2010/0098704, the antibodies of which are incorporated herein by reference. Non-limiting examples of anti-CD40 antibodies are lucatumumab and dacetuzumab. A non-limiting example of an anti-CD304 antibody is vesencumab.

Conjugation techniques for linking antigen-binding moieties are known in the art, for example, as described in Ansell et al., Methods Mol. Med., 25:51-68, 2000; US 2002/0025313; U.S. Pat. Nos. 6,379,699; and 5,059,421.

### Polypeptides

The targeting moiety can be a polypeptide having an affinity for cells (e.g., having an affinity for a cell type, e.g., a dendritic cell). Non-limiting examples of polypeptides are RGD peptide, rabies virus glycoprotein (RVG), and DC3 peptide. Alternatively, the polypeptide may be a TLR2 agonist, e.g., MALP-2 lipoprotein, MALP-404 lipoprotein, OspA, a porin, LcrV, Hsp60, glycoprotein gH/gL, or glycoprotein gB.

Conjugation techniques for linking peptides are known in the art, for example, as described in Ansell et al., Methods Mol. Med., 25:51-68, 2000; US 2002/0025313; U.S. Pat. Nos. 6,379,699; and 5,059,421.

### PAMPs

The targeting moiety may be a PAMP. PAMPs are known in the art, e.g., a CpG ODN. CpG ODNs are generally divided into three classes: class A, class B, and class C. Class A CpG ODNs typically contain poly-G tails with phosphorothioate backbones at the 3′- and 5′-termini and a central palindromic sequence including a phosphate backbone. Class A CpG ODNs typically contain CpG within the central palindromic sequence. Class B CpG ODNs typically include fully phosphorothioated backbone, and the sequence at the 5′ end of class B CpG ODNs is often critical for TLR9 activation. Class C CpG ODNs include a fully phosphorothioated backbone with a 3′-end sequence enabling formation of a duplex. A PAMP may be covalently linked to a supramolecular structure using techniques and methods known in the art.

### Methods of Assembly

The present invention features supramolecular structures (e.g., a lipid based supramolecular structure, such as a liposome) that include a plurality of enzymes packaged therein. Described herein are methods of assembly to produce structures (e.g., liposomes) containing the enzymes (e.g., Lysin A, Lysin B, isoamylase, and/or α-amylase, e.g., having at least 85% sequence identity to a sequence of Table 1). Individual proteins can be overexpressed in any suitable recombinant expression system (e.g., E. coli) and extracted from the cells via lysis. In some embodiments, the crude extract from the cells may be purified, e.g., via column chromatography. Following purification, enzyme component concentrations may be standardized for subsequent encapsulation, e.g., into liposomes. The concentrations may be standardized, e.g., at from 0.1 mg/mL to 10 mg/ml (e.g., 0.1 mg/mL to 1 mg/mL, e.g., 0.2 mg/mL, 0.3 mg/mL, 0.4 mg/mL, 0.5 mg/mL, 0.6 mg/mL, 0.7 mg/mL, 0.8 mg/mL, 0.9 mg/mL, or 1 mg/mL, e.g., from 1 mg/mL to 10 mg/mL, e.g., 2 mg/ml, 3 mg/mL, 4 mg/mL, 5 mg/mL, 6 mg/mL, 7 mg/mL, 8 mg/mL, 9 mg/mL, or 10 mg/mL). In some embodiments, the concentrations may be standardized at 0.33 mg/mL.

Lipids may then be mixed with the enzymes for formulate the liposomes. For example, lipids at a total concentration of 0.1 mg/mL to 10 mg/mL (e.g., 0.1 mg/mL to 1 mg/mL, e.g., 0.2 mg/mL, 0.3 mg/mL, 0.4 mg/mL, 0.5 mg/mL, 0.6 mg/mL, 0.7 mg/mL, 0.8 mg/mL, 0.9 mg/mL, or 1 mg/mL, e.g., from 1 mg/mL to 10 mg/mL, e.g., 2 mg/ml, 3 mg/mL, 4 mg/mL, 5 mg/mL, 6 mg/mL, 7 mg/mL, 8 mg/mL, 9 mg/mL, or 10 mg/mL, e.g., from 1 mg/mL to 5 mg/mL) may be mixed with 1 mg/mL to 10 mg/ml His-tagged proteins. The lipids may include, for example, one or more of DOPC, DOPE, DOPS, and cholesterol. In some embodiments, the lipids include DOPC, DOPE, DOPS, and cholesterol in a ratio of 10:10:3:4 ratio. The lipids may be resuspended in a suitable organic solvent (e.g., ethanol) and mixed with the proteins (e.g., at a ratio of from 20:1 to 1:1, e.g., 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or 1:1, e.g., 3:1 to 10:1, e.g., 3:1 to 8:1 aqueous: organic). The mixture may be mixed at a flow rate of 1 ml/minute to 30 mL/minute, e.g., 15 mL/minute, e.g., using a suitable system, such as the NANOASSEMBLR® IGNITE™ system (Precision Nanosystems). The organic layer may be removed by dialysis, e.g., against excess (e.g., 1000X) volumes of formulation buffer, e.g., for at least 30 minutes (e.g., for 1 hour) at a suitable temperature, such as room temperature. Liposomes may then be analyzed by gel electrophoresis (e.g., SDS-PAGE), dynamic light scattering, intrinsic fluorescence, and/or static light scattering and tested for relevant activity. Such assays may help confirm the encapsulation of the enzymes and purity of the enzymes and the liposomes.

The compositions containing the supramolecular structures (e.g., liposomes) may be formulated with one or more excipients. For example, the composition (e.g., a supramolecular structure, e.g., a liposome containing a cocktail of lytic enzymes) may be encapsulated and/or formulated in buffer, such as glycine, Tris, sodium citrate, sodium acetate, and MES, e.g., at a concentration of 10 mM to 200 mM, e.g., 50 mm to 150 mm, e.g., 10 mM, 20 mm, 30 mm, 40 mm, 50 mm, 60 mm, 70 mm, 80 mm, 90 mM, 100 mM, 110 mM, 120 mM, 130 mM, 140 mM, 150 mM, 160 mM, 170 mM, 180 mM, 190 mM, or 200 mM. The composition may be formulated at a pH of from 5 to 11 (e.g., a pH of 5 to 6, e.g., 5.1, 5.2, 5.3, 5.4, 5.5, 5.6, 5.7, 5.8, 5.9, or 6, e.g., 6 to 11, e.g., 6.5, 7, 7.5, 8, 8.5, 9, 9.5, 10, 10.5, or 11). The composition may further include one or more excipients, such as CaCl₂), arginine, NaCl, sodium citrate, MgCl₂, or glycerol. In some embodiments, the composition includes, for example, 5 mM to 10 mM CaCl₂), 0 to 50 mM arginine, 0 to 200 mM NaCl, 0 mM to 1 mM sodium citrate, 0 to 1 mM MgCl₂, and/or 10-30% glycerol. In some embodiments, the composition includes 50 mM glycine, pH 8.5, 7.5 mM CaCl₂), 0.5 mM MgCl₂, 200 mM NaCl, 0.33 mM sodium citrate, and 10% glycerol. The formulation may further include TWEEN, e.g., TWEEN-80. Once the compositions are in a preferred storage or activity buffer, they can be used in treatment or for assays or saved for subsequent use.

### Methods of Treatment

The antibacterial lytic proteins described herein are preferably formulated into pharmaceutical compositions for administration to human subjects for the treatment of a disease or condition, such as a bacterial infection (e.g., actinomycetia infection, e.g., corynebacteriales or propionibacteriales), e.g., intracellular bacterial infection, e.g., mycobacterial infection, e.g., NTM infection). In particular, the compositions and methods described herein are useful for treating bacterial infections caused by actinomycetia, e.g., corynebacterials and propionibacteriales, due to their similar envelope structure. Bacterial infections may occur in otherwise healthy subjects. Alternatively, the bacterial infection may occur in a subject with another comorbidity or disease. For example, a subject with a weakened immune system may be more susceptible to a bacterial infection.

Mycobacterial infections caused by NTM are bacteria that are normally present in the environment. Inhalation of these bacteria may cause disease in both healthy patients and those with compromised immune systems. NTM disease most often affects the lungs in adults, but it may also affect any body site. Some subjects are at higher risk of getting an NTM infection and developing disease. People who have an existing lung disease such as bronchiectasis (enlargement of airways), chronic obstructive pulmonary disease (COPD), cystic fibrosis, alpha-1 antitrypsin deficiency or who have had prior infections such as tuberculosis are at increased risk of pulmonary NTM disease. Subjects with advanced HIV infection (CD4<50) or immune-related genetic disorders (e.g., interferon-gamma deficiency or receptor deficiency, interleukin-12 deficiency) may develop pulmonary disease as part of a disseminated (e.g., widespread in the body) NTM infection. The subject to be treated may have any of the foregoing indications, e.g., in addition to a bacterial infection.

The methods compositions and methods described herein may be used to reduce a level of infection. For example, the methods may decrease a level of infection (e.g., number of bacteria or size of infection), as compared to a reference. For example, the infection may decrease by 5%, 10%, 15%, 20%, 25%, 30%, 35%, 40%, 45%, 50%, 55%, 60%, 65%, 70%, 75%, 80%, 85%, 90%, 95%, or 100%.

### Pharmaceutical Compositions

The antibacterial agents described herein are preferably formulated into pharmaceutical compositions for administration to human subjects in a biologically compatible form suitable for administration in vivo.

The compositions described herein may be administered to a subject in a variety of forms depending on the selected route of administration, as will be understood by those skilled in the art. The compositions described herein may be administered, for example, by any route that allows the composition (e.g., supramolecular structure, e.g., liposome, micelle, or LNP) to reach the target cells. The composition may be administered, for example, by oral, parenteral, intrathecal, intracerebroventricular, intraparenchymal, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, intracerebroventricular, intraparenchymal, rectal, and topical modes of administration. In one embodiment, the composition is administered via aerosol. Parenteral administration may be by continuous infusion over a selected period of time. In some preferred embodiments, the compositions described herein are administered via inhalation.

Administration of more than one antibacterial agent may be by the same route or by different routes and may occur sequentially or substantially simultaneously. For example, a first antibacterial agent of the combination may be administered by intravenous injection while a second therapeutic agent of the combination may be administered orally.

Certain compositions described herein may be administered, e.g., by inhalation. Inhalation may be oral inhalation or nasal inhalation. An inhalable composition described herein may be provided as a liquid dosage form or dry powder dosage form. A dry powder composition may be, e.g., administered by inhalation as is or after reconstitution in a vehicle (e.g., saline (e.g., isotonic saline), phosphate-buffered saline, or water).

Inhalable dry powder dosage forms may be prepared from liquid compositions described herein by drying (e.g., by freeze drying, spray drying, spray-freeze drying, or supercritical fluid technology). Inhalable dry powder dosage forms described herein may include a carrier (e.g., lactose, sucrose, mannitol, and the like), cryoprotectant (e.g., trehalose, mannitol, and the like), and/or antiadherent (e.g., glycine, L-leucine, serine, and the like). Inhalable dry powder dosage forms described herein may be administered using dry powder inhalers. Dry powder inhalers are known in the art and may or may not include a propellant. Non-limiting examples of dry powder inhalers can be found in Newman, Expert Opin. Biol. Ther., 4:23-33, 2004, the disclosure of which is incorporated herein by reference in its entirety.

Inhalable liquid dosage forms (e.g., aerosol formulations) described herein may be prepared using techniques and methods useful in the preparation of liquid compositions containing supramolecular structures. Inhalable liquid dosage forms typically include a suspension of the supramolecular structures described herein in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, e.g., a single dose nasal inhaler or an aerosol dispenser fitted with a metering valve which is intended for disposal after use. Where the dosage form contains an aerosol dispenser, it will contain a propellant, which can be a compressed gas, e.g., compressed air or an organic propellant, e.g., hydrofluoroalkane. The inhalable liquid dosage forms may be administered using a nebulizer. The process of pneumatically converting a bulk liquid into small droplets is called atomization. The operation of a pneumatic nebulizer requires a propellant as the driving force for liquid atomization. Various types of nebulizers are described in Respiratory Care, 45:609-622, 2000, the disclosure of which is incorporated herein by reference in its entirety. Alternatively, an inhalable liquid dosage form described herein may be administered using a metered-dose inhaler. Metered-dose inhalers are known in the art and typically include a canister, actuator, and a metering valve.

A composition described herein may be orally administered, for example, with an inert diluent or with an assimilable edible carrier, or it may be enclosed in hard-or soft-shell gelatin capsules, or it may be compressed into tablets, or it may be incorporated directly with the food of the diet. For oral therapeutic administration, a composition described herein may be incorporated with an excipient and used in the form of ingestible tablets, buccal tablets, troches, capsules, elixirs, suspensions, syrups, and wafers. A composition described herein may also be administered parenterally. A composition described herein may also be administered microneedle injection. Solutions of a composition described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF 36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions suitable for buccal or sublingual administration include tablets, lozenges, and pastilles, where the active ingredient is formulated with a carrier, such as sugar, acacia, tragacanth, gelatin, and glycerin. Compositions for rectal administration are conveniently in the form of suppositories containing a conventional suppository base, such as cocoa butter.

In some embodiments, the compositions described herein are formulated with one or more excipients. For example, the composition (e.g., a supramolecular structure, e.g., a liposome containing a cocktail of lytic enzymes) may be encapsulated and/or formulated in buffer, such as glycine, Tris, sodium citrate, sodium acetate, and MES, e.g., at a concentration of 10 mM to 200 mM, e.g., 50 mm to 150 mm, e.g., 10 mM, 20 mm, 30 mm, 40 mm, 50 mm, 60 mm, 70 mm, 80 mm, 90 mM, 100 mM, 110 mM, 120 mM, 130 mM, 140 mM, 150 mM, 160 mM, 170 mM, 180 mM, 190 mM, or 200 mM. The composition may be formulated at a pH of from 5 to 11 (e.g., a pH of 5, 5.5, 6, 6.5, 7, 7.5, 8, 8.5, 9, 9.5, 10, 10.5, or 11). The composition may further include one or more excipients, such as CaCl₂), arginine, NaCl, sodium citrate, MgCl₂, or glycerol. In some embodiments, the composition includes, for example, 5 mM to 10 mM CaCl₂), 0 to 50 mM arginine, 0 to 200 mM NaCl, 0 to 1 mM sodium citrate, 0 to 1 mM MgCl₂, and/or 10-30% glycerol. In some embodiments, the composition includes 50 mM glycine, pH 8.5, 7.5 mM CaCl₂), 0.5 mM MgCl₂, 200 mM NaCl, 0.33 mM sodium citrate, and 10% glycerol. The formulation may further include TWEEN, e.g., TWEEN-80.

The composition described herein may be administered to an animal, e.g., a human, alone or in combination with pharmaceutically acceptable carriers, as noted herein, the proportion of which is determined by the solubility and chemical nature of the composition, chosen route of administration, and standard pharmaceutical practice.

The dosage of the compositions, e.g., a composition including a lytic protein, described herein, can vary depending on many factors, such as the pharmacodynamic properties of the antibacterial lytic proteins, the mode of administration, the age, health, and weight of the recipient, the nature and extent of the symptoms, the frequency of the treatment, and the type of concurrent treatment, if any, and the clearance rate of the composition in the animal to be treated. The compositions described herein may be administered initially in a suitable dosage that may be adjusted as required, depending on the clinical response. In some embodiments, the dosage of a composition, e.g., a composition including a lytic protein, is a prophylactically or a therapeutically effective amount. Furthermore, it is understood that all dosages may be continuously given or divided into dosages given per a given time frame. The composition can be administered, for example, every hour, day, week, month, or year. In some embodiments, the composition may be administered continuously or systemically.

### Combination Therapies

The pharmaceutical compositions described herein may be administered as part of a combination therapy. A combination therapy means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. Sequential or substantially simultaneous administration of each therapeutic agent can be by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection or via aerosolization while a second therapeutic agent of the combination may be administered orally.

In any of the combination embodiments described herein, the first and second therapeutic agents may be administered simultaneously or sequentially, in either order. The first therapeutic agent may be administered immediately, up to 15 minutes, up to 30 minutes, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

The pharmaceutical compositions described herein may further include an additional antibacterial agent that is administered in conjunction with the supramolecular structure that includes an antibacterial lytic protein.

The compositions and methods described herein may further include treatment for an underlying lung condition, e.g., that may be exacerbated by a bacterial infection, e.g., NTM infection. Suitable lung therapies include, without limitation, airway clearance, nebulizers, respirators, and inhalers, e.g., steroid inhalers.

### Antibiotics

The additional antibacterial agent may be an antibiotic. Suitable antibiotics include, without limitation, penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, chlorhexidine, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, erythromycin, azithromycin, clarithromycin, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, sitafloxacin, metronidazole, daptomycin, garenoxacin, ramoplanin, faropenem, polymyxin, tigecycline, AZD2563, trimethoprim, ethambutol, rifamycin, and rifampin. In some embodiments, multiple antibiotics are administered in combination with the compositions described herein. In some embodiments, the antibiotic is a cephalosporin, carbapenem (e.g., biapenem), penicillin, a macrolide, an aminoglycoside, or a fluoroquinolone. In some embodiments, the antibiotic is selected from the group consisting of thiacetazone, sq-109, bedaquiline, delamanid, pyrazinamide, and isoniazid.

In some embodiments, the antibiotic is a macrolide (e.g., azithromycin, clarithromycin, erythromycin). In some embodiments, the antibiotic is an aminoglycoside (e.g., kanamycin A, amikacin, tobramycin, dibekacin, gentamicin, sisomicin, netilmicin, neomycin (e.g., neomycin B, C, or E), streptomycin, or plazomicin).

Advantageously, in some embodiments, the synergy with the co-administered therapeutic agents may permit the antibiotic to be administered at a dose that would be subtherapeutic, if administered without the other therapeutic agents.

The antibiotic may be formulated with the supramolecular structure containing the antibacterial lytic proteins. The antibiotic may be administered as a separate pharmaceutical composition. The antibiotic may be administered at a different time than the pharmaceutical composition containing the supramolecular structure with phage. In some preferred embodiments, the additional antibiotic is amikacin. The amikacin may be liposomal amikacin that is formulated, e.g., for inhalation.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1

### Background

Mycobacteria possess unique resistances to antibiotics in part due to a complicated cell-surface envelope that contains multiple layers from inside out plasma membrane PM, peptidoglycan (PG), arabinogalactan (AGL), mycolic acid layer (MA), and capsule layer (CL).

Endolytix Cocktail 1 (EC1) was built upon four enzymes that catalytically attack three layers within the mycobacterial envelope. The first enzyme is Lysin A, a phage encoded protein that represents a conserved family of proteins that catalyze lysis of the PG layer for bacteriophage progeny release. Mycobacteria also show a diversity in the chemical linkages in their PG layers and are able to control the ratio of 3,3 and 4,3 linked stem peptides that crosslink the many glycan layers that go on to form the PG layer.

The second enzyme, Lysin B is exclusive to actinomycetia phages that include, for example, mycobacteriophages and related host species discussed above whose hosts contain a MA layer. Lysin B is an esterase that hydrolyzes the linkage between arabinogalactan and mycolic acids.

The mechanism of action for Lysin A and Lysin B naturally attacks a mycobacterial host from the inside out. The problem of providing access to the substrates of Lysin A and Lysin B exogenously was addressed by adding enzymes that would degrade the M. tuberculosis capsule. The capsule degrading enzymes (capsulases), hydrolyze α-1-4 and α-1,6 linked glucose within the capsular polysaccharides. The structure of polysaccharides in the mycobacterial envelope CL consists of heptamer α-1,4-glycan chains linked by two α-1,6-glycosidic connections to create a meshwork of crosslinked heptamers. Cleaving the capsule polysaccharides exposes the mycolic acid and arabinogalactan layer allowing easier access to the target layers (i.e., substrates) for both Lysin A and Lysin B. α-amylase (EC 3.2.1.1) is a hydrolase that cleaves α-1,4-glycan chains. Isoamylase (EC 3.2.1.68) dissolves α-1,6-glycosidic chains that link together α-1,4-glycan chains.

This unique combination of enzymes allows degradation of the mycobacterial envelope from the outside in, reversing traditional phage-based approaches while also avoiding the inactivation of the many genome surveillance pathways that cleave the bacteriophage genome and kill bacteriophage.

Another problem for a mycobacterial therapeutic is being able to kill intracellular bacteria. We addressed the intracellular delivery of enzymes by loading them into liposomes that contained a phospholipid phosphatidylserine (PS) that stimulates macrophages to engulf such cells as it is an indication of apoptosis when PS appears on the outer leaflet of mammalian cells.

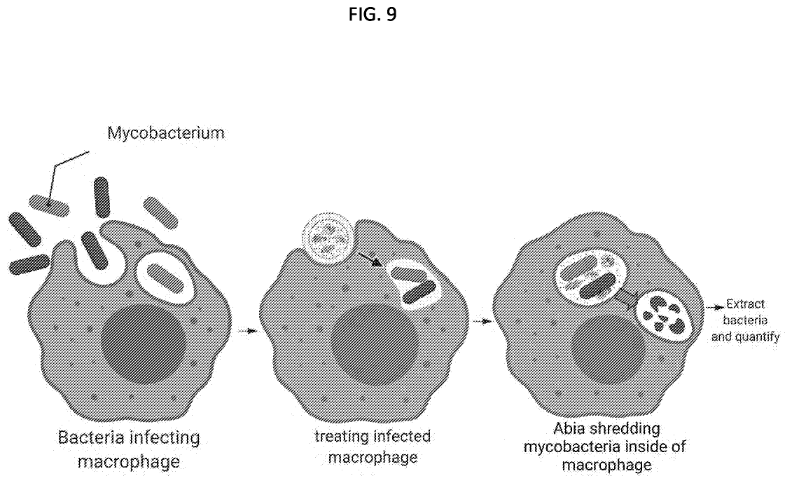

We demonstrated destruction of the mycobacterial envelope with an enzyme cocktail that was designed to attack the capsule, the linkage between the MA and the AGL, and PG. We also demonstrated a mechanism of action for EC1 that results in mycobacterial cell death by fragmentation into subcellular fragments. We also demonstrated the ability of EC1 to rescue macrophages from the apoptotic phenotype associated with mycobacterial infected macrophages (MIM). Here we present the development and performance of EC1 on non-tuberculosis mycobacteria (NTM) species in vitro and ex vivo (FIG. 9).

### In Silico and Biochemical Identification of Lysin A

Lysin A genes are modular and typically comprise two domains: an N-terminal catalytic domain and a C-terminal protein binding domain. Lysin A typically catalyzes degradation of the peptidoglycan layer of the mycobacterial envelope. To select a Lysin A for EC1, we identified unique functional domains using computational screening and validated the identified genes with biophysical characteristic testing.

We collected all phage genes from PhagesDB (371,279 genes; accessed on Apr. 21, 2021) and performed a bioinformatic analysis using BLAST. Genes annotated as Lysin A were subset from the gene set and used to create a protein homology database. The remaining genes were compared against the Lysin A protein database for homology using BLASTp. Queries with at least 90% sequence identity to a target Lysin A were retained resulting in the addition of 78 non-labeled genes to the already 1753 labeled Lysin A genes. Through additional quality control testing, we discovered two Lysin A genes mislabeled as Lysin B, thus bringing the total number of Lysin A targets to 1833. The identified domain sequences within each Lysin A were extracted, resulting in 27 unique domain architectures. These unique domain architectures were cloned into a modified pET21a vector and screened for yield. Nine of the plasmids were high yield and all nine high yield enzymes were capable of hydrolyzing peptidoglycan. The thermostability and aggregation properties of the nine enzymes were tested using the UNCLE® device from Unchained Labs. Three enzymes possessed an aggregation and melting point temperature above 40° C. We selected the enzyme with the highest aggregation and melting point as the Lysin A component of EC1. From 371,279 genes to just three candidates, we screened and experimentally validated the biophysical properties of the Lysin A in EC1.

### Source of Additional Components in EC1

The pipeline applied to discover the Lysin A peptide was applied to Lysin B peptide sequences resulting in the identification of 15 unique Lysin B architectures. Among these unique architectures was a candidate from preliminary screening. This Lysin B gene displayed both catalytic activity and excellent yield. The aggregation and melting temperatures were 52.3° C. and 58.2° C., respectively.

We commercially sourced α-amylase and expressed fungal isoamylases with known crystal structures to test the hypothesis that EC1 can penetrate the outer leaflet allowing the Lysin A and Lysin B access to their target substrate.

### Liposome Packaging of EC1

Enzyme component (i.e., of SEQ ID NOs: 2, 184, 392, and 394) concentrations were standardized at 0.33 mg/mL. The protein solution was packaged into liposomes or related lipid drug delivery vehicles using Precision Nanosystems NANOASSEMBLR® IGNITE™.

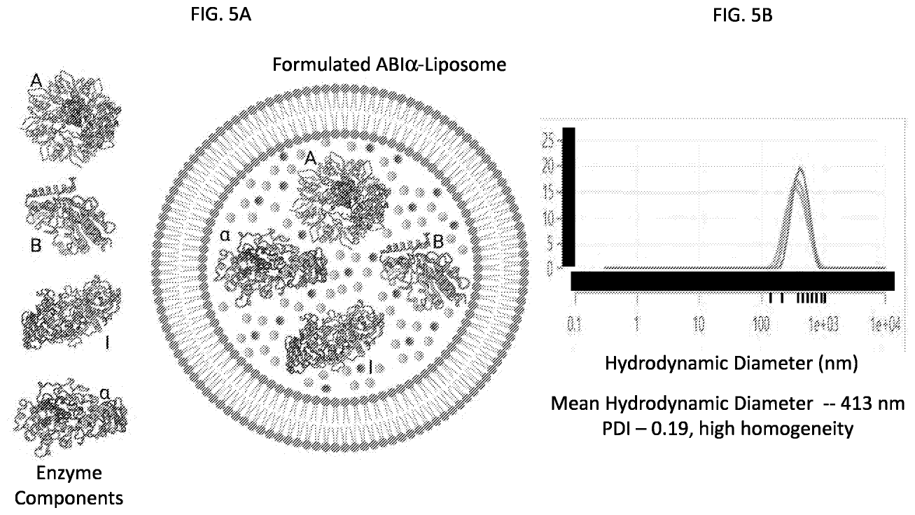

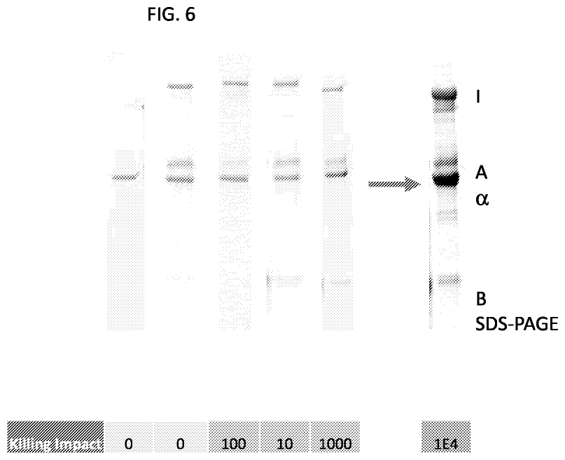

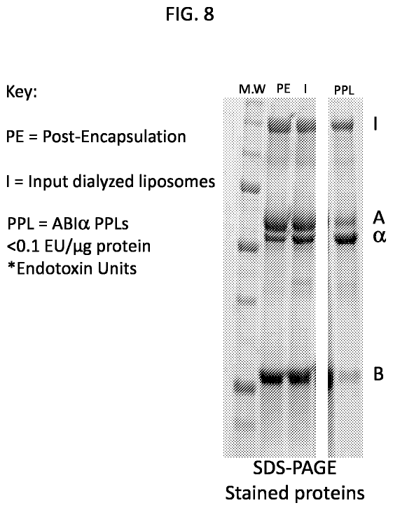

Lipids at a total concentration of 1-5 mg/mL (DO-phosphatidylcholine, DO-phosphatidylethanolamine, DO-phosphatidylserine, and cholesterol in a 1:1:0.3:0.4 ratio), resuspended in EtOH were mixed with 1-10 mg/ml his-tagged proteins (which can include capsulases, lysins) at a ratio of 3:1 aqueous: organic at a flow rate of 15 mL/minute using the NANOASSEMBLR® IGNITE™ system (Precision Nanosystems). Liposomes were then analyzed by SDS-PAGE (8-16% Gradient Mini-Protean Pre-cast gels; Bio-Rad), dynamic light scattering (Zetasizer Ultra; Malvern), intrinsic fluorescence (Uncle; Unchained Labs), and static light scattering (Uncle) and tested for relevant antibiotic activity (FIGS. 6 and 8). Dynamic light scattering showed a mean hydrodynamic diameter of 413 nm and a PDI of 0.19, indicating high homogeneity (FIGS. 5A and 5B).

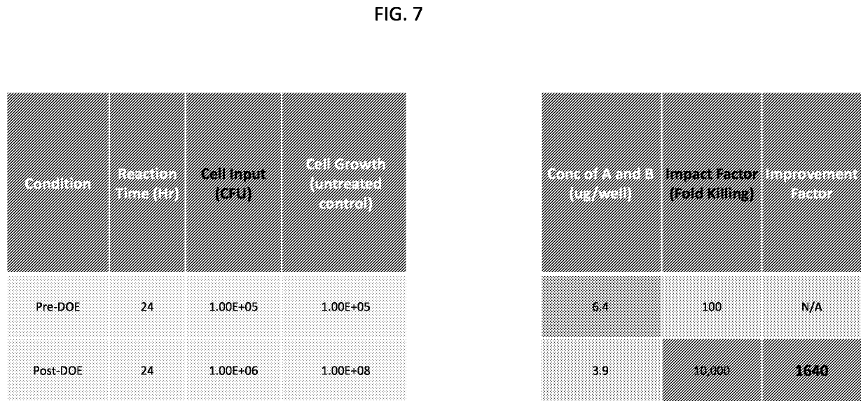

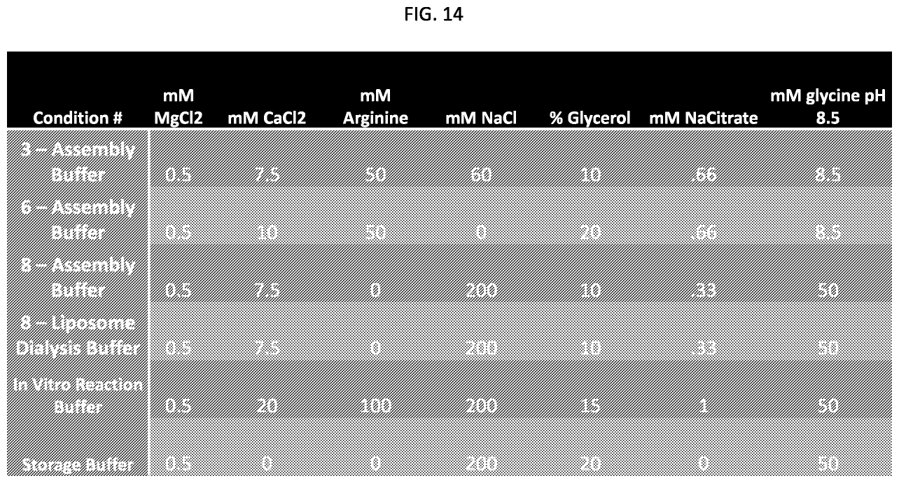

The formulation buffer consisted of at least one buffer component from Glycine, Tris, Sodium Citrate, Sodium Acetate, and MES at a concentration of 50-150 mM and at a pH between 5 and 11, with one or more excipients (5-10 mM CaCl₂), 0-50 mM Arginine, 0-200 mM NaCl, 0.33-1 mM Sodium Citrate, 0.5 mM MgCl₂, and 10-30% glycerol). One formulation included 50 mM glycine, pH 8.5, 7.5 mM CaCl₂, 0.5 mM MgCl₂, 200 mM NaCl, 0.33 mM Sodium Citrate, and 10% glycerol (FIG. 14). This formulation (Formulation 8) showed enhanced killing relative to other conditions (FIG. 7).

### Growth Curve Identification Algorithm

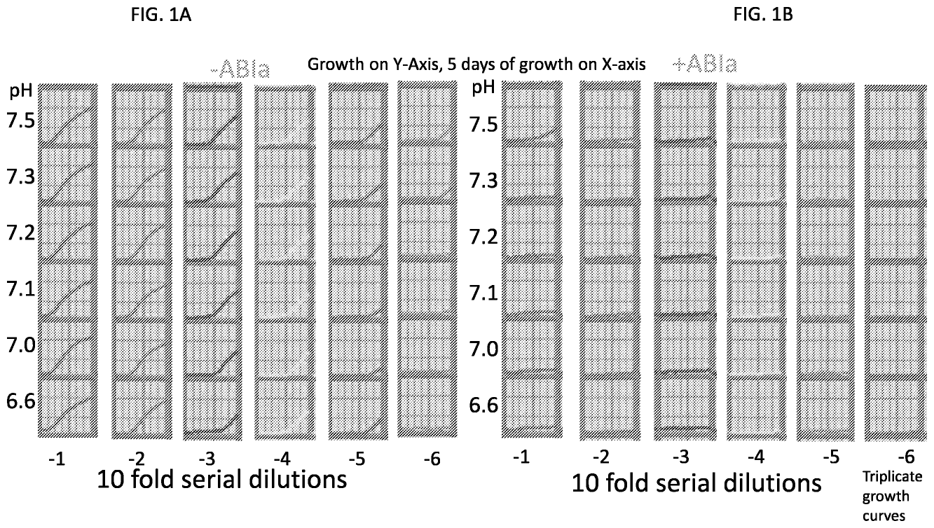

To identify growth of NTM in the presence of our enzymes, we collected kinetic optical density information using the OMNILOG® from Biolog. Observations were collected from 96-well plates every 30 minutes for 121 hours resulting in 242 observations per well. To identify growth from kinetic data, the observation values were smoothed, and the second derivative was calculated to determine an inflection point. We calculated a line tangent to an observed inflection point and identified the x position where the tangent line crossed the y-intercept. The y-value of the x position of the tangent line was used as the inflection start value. We calculated a threshold value by dividing the y-value of the inflection point by the inflection start value. If the calculated threshold value was above 1.4, then the well was labeled as growing NTM. An increase in time of inflection start values also demonstrated growth inhibitory effects. Additionally, potential contamination was identified by inflection points that demonstrated growth within 24 hours because NTM grows slowly relative to other bacteria (FIGS. 1A and 1B).

Mycobacterial cells aggregate and make traditional methods inaccurate for quantification. We introduced enzymes and sonication with optimization in time and frequency and demonstrated more ideal dilution behavior when not aggregated, a big problem in the field.

### Impact Factor Calculations

To calculate the logarithmic growth changes when applying EC1, we created a series of dilution experiments and measured kinetic growth. We compared binary growth calls across dilutions in the initial seeding, untreated controls, and treated samples. Impact factor was calculated by the binary dilution factor of the untreated growth over the treated growth (FIG. 6).

### Component Analysis In Vitro

We analyzed the effect of individual enzymes and all possible component combinations on NTM growth start times. Concentrations of the combinations tested ranged from 0.0125 μg to 5 μg.

### Imaging of EC1 in Macrophages

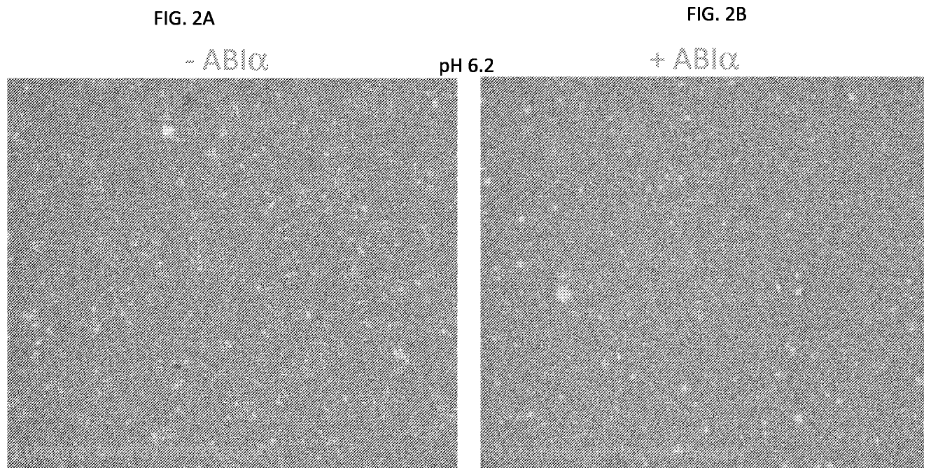

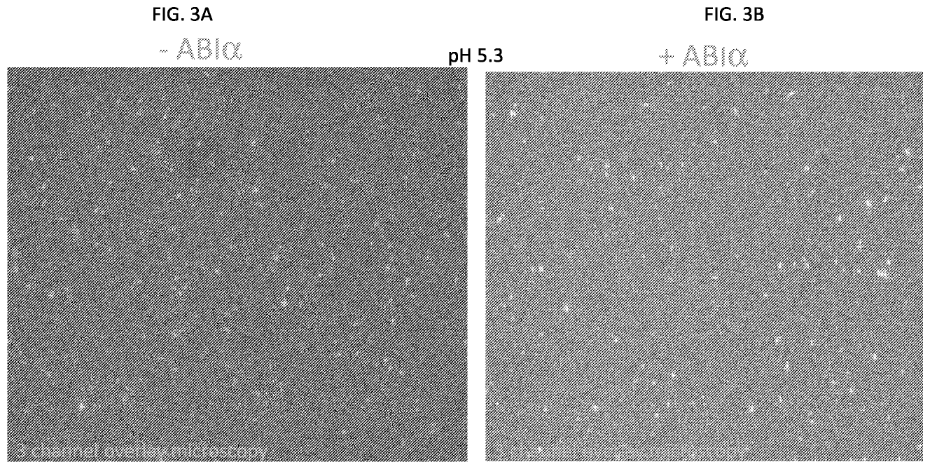

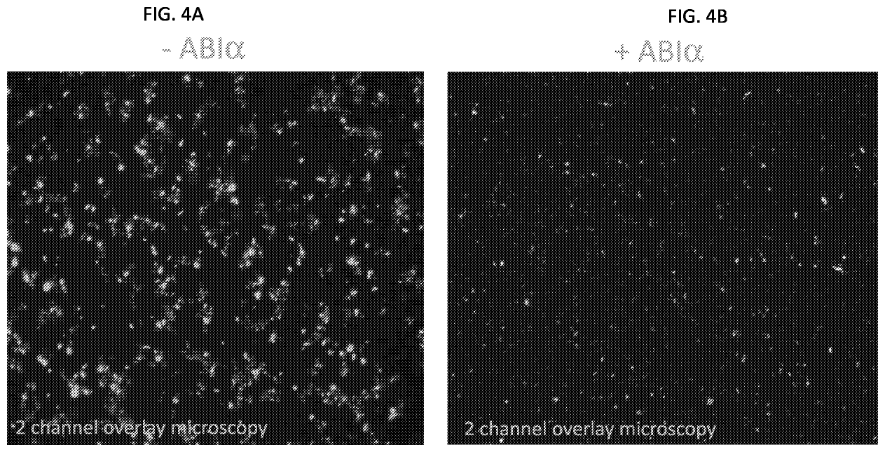

FIGS. 2A and 2B are bright field images of mycobacterial bacilli rod shaped cells that are shredded by ABIα into subcellular fragments that contain stainable lipids giving rise to red staining bodies that are separated from the genomic DNA. Th experiment was performed at pH 6.2. FIG. 2A shows untreated mycobacterial cells without fragments. Lipids are shown in red, and DNA is shown in green. Green staining DNA indicates dye access afforded by a compromised cell envelope. Intact cells do not readily uptake red dye. FIG. 2B shows ABIα treated cells and fragments. Lipids are shown in red, and DNA is shown in green. Subcellular fragments light up with the lipophilic red dye. The green DNA is spread out as compared to FIG. 2A. FIGS. 3A and 3B show a similar experiment, with shredding observed at pH 5.3. FIGS. 4A and 4B are bright field images showing ABIα mechanism by shredding mycobacteria into fragments. FIG. 4A shows untreated mycobacterial cells without fragments. FM4-64 is a red dye that stains lipids, envelop, and lipid bodies. FIG. 4A shows treated mycobacterial cells with fragments. Cytox-Green stains DNA in cells with compromised envelopes.

### Cell Killing Assays

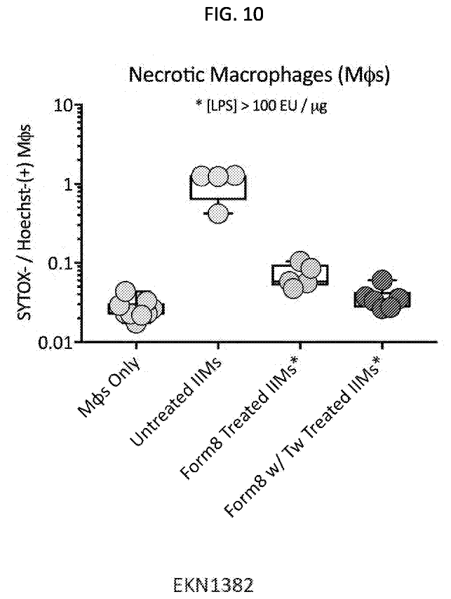

FIG. 10 is graph showing ABIα-PPL formulation-8 treatment of J774A. 1 mouse macrophages (Mos) uninfected and infected with Mycobacterium abscessus (MOI=10:1). The amount of necrotic cell death for Mos was determined to assess safety and efficacy of ABIα-PPL treatment of mycobacterial infections. The level of necrosis for Mos was quantified as a ratio of the number of cells, stained with SYTOX™ Green Nucleic Acid Stain (Invitrogen™) in the population, stained with Hoechst-33342 (Invitrogen™), counted per FOV (N=2-3 per well) with HCIA platform (BZ-X800; Keyence). Decreases in necrosis were seen in both uninfected (Form8 Mos Only) and infected (Form8 Treated IIMs) Formulation-8 treatment groups compared with the untreated control group (Untreated IIMs).

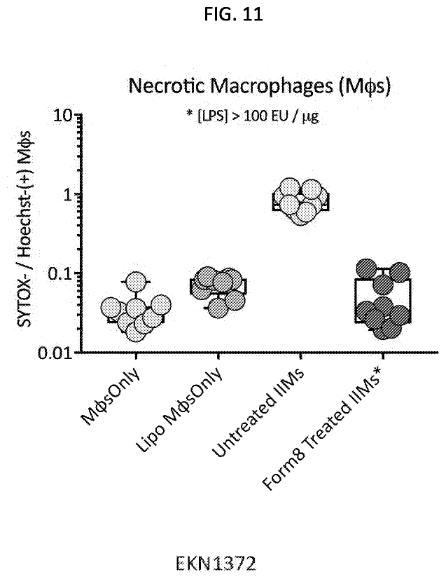

FIG. 11 is a graph showing ABIα-PPL formulation-8 treatment of J774A.1 mouse macrophages (Mos) uninfected and infected with Mycobacterium abscessus (MOI=10:1). The amount of necrotic cell death for Mos was determined to assess safety and efficacy of ABIα-PPL treatment of mycobacterial infections. The level of necrosis for Mos was quantified as a ratio of the number of cells, stained with SYTOX™ Green Nucleic Acid Stain (Invitrogen™) in the population, stained with Hoechst-33342 (Invitrogen™), counted per FOV (N=3 per well) with HCIA platform (BZ-X800; Keyence). Decreases in necrosis were observed in both Formulation-8 treated groups (Mos Only and Form8 Treated IIMs), and uninfected Mos treated with empty liposomes (Lipo MosOnly), compared with the untreated control group (Untreated IIMs).

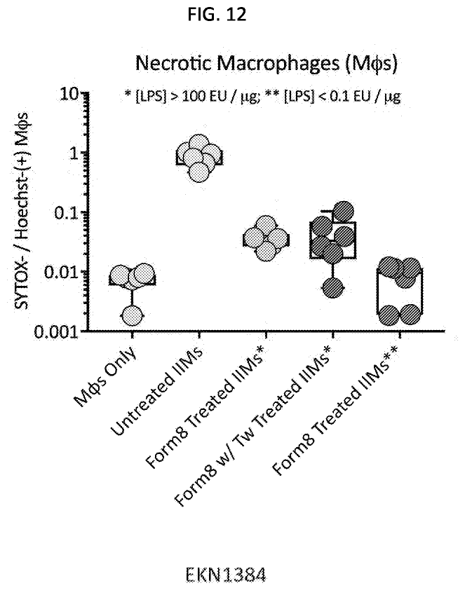

FIG. 12 is a graph showing ABIα-PPL treatment of J774A.1 mouse macrophages (Mos) uninfected and infected with Mycobacterium abscessus (MOI=10:1). The level of necrosis and oxidative stress in Mos was quantified to assess safety and efficacy of ABIα-PPL treatment of mycobacterial infections. The level of necrosis for Mos was calculated as a ratio of the number of cells, stained with SYTOX™ Green Nucleic Acid Stain (Invitrogen™) in the population, stained with Hoechst-33342 (Invitrogen™), counted per FOV (N=2-3 per well) with HCIA platform (Keyence).

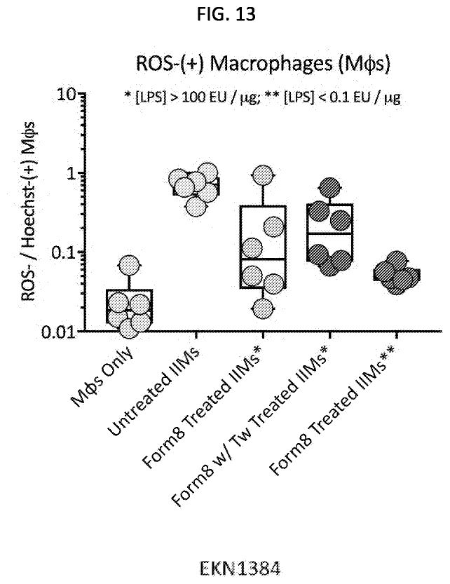

FIG. 13 is a graph showing the level of oxidative stress for Mos was quantified as a ratio of the number of cells, stained with CellROX™ (Invitrogen™) in the population, stained with Hoechst-33342 (Invitrogen™), counted per FOV (N=2-3 per well) with HCIA platform (BZ-X800; Keyence). Decreases in necrosis were observed in all the treatment groups relative to the untreated control group (Untreated IIMs). Decreases in oxidative stress were seen in two treatment groups (Mos Only and Form8 Treated IIMs**) compared with untreated IIMs.

### CONCLUSION

In this study, we showed EC1 can degrade the mycobacterial envelope and kill NTM both in vitro and inside of macrophages. EC1 differs from antibiotic small molecule disruptions by degrading the mycobacterial envelope regardless of microbial replication status. EC1 disrupts with multiple cleavage sites at different levels of the mycobacterial envelope, destabilizing the envelope and sterilizing the bacteria.

### OTHER EMBODIMENTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and that this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

1. A composition comprising a supramolecular structure comprising two or more of:
(a) a Lysin A comprising an amino acid sequence having at least 85% sequence identity to any one of SEQ ID NOS: 1-182;
(b) a Lysin B comprising an amino acid sequence having at least 85% sequence identity to any one of SEQ ID NOs: 183-241;
(c) an isoamylase comprising an amino acid sequence having at least 85% sequence identity to any one of SEQ ID NOs: 242-392; and
(d) an α-amylase comprising an amino acid sequence having at least 85% sequence identity to any one of SEQ ID NOS: 393-445.

2. The composition of claim 1, comprising two or more of:
(a) a Lysin A comprising an amino acid sequence having at least 85% sequence identity to SEQ ID NO: 1 or SEQ ID NO: 2;
(b) a Lysin B comprising an amino acid sequence having at least 85% sequence identity to SEQ ID NO: 183 or SEQ ID NO: 184;
(c) an isoamylase comprising an amino acid sequence having at least 85% sequence identity to SEQ ID NO: 242 or SEQ ID NO: 243; and
(d) an α-amylase comprising an amino acid sequence having at least 85% sequence identity to any one of SEQ ID NOS: 393-398.

3. The composition of claim 1, comprising:
(a) a Lysin A comprising an amino acid sequence having at least 85% sequence identity to any one of SEQ ID NOs: 1-182; and
(b) a Lysin B comprising an amino acid sequence having at least 85% sequence identity to any one of SEQ ID NOs: 183-241.

4. The composition of claim 3, comprising:
(a) a Lysin A comprising an amino acid sequence having at least 85% sequence identity to SEQ ID NO: 1 or SEQ ID NO: 2; and
(b) a Lysin B comprising an amino acid sequence having at least 85% sequence identity to SEQ ID NO: 183 or SEQ ID NO: 184.

5. The composition of claim 1, comprising:
(a) a Lysin A comprising an amino acid sequence having at least 85% sequence identity to any one of SEQ ID NOs: 1-182; and
(b) an isoamylase comprising an amino acid sequence having at least 85% sequence identity to any one of SEQ ID NOs: 242-392.

6. The composition of claim 5, comprising:
(a) a Lysin A comprising an amino acid sequence having at least 85% sequence identity to SEQ ID NO: 1 or SEQ ID NO: 2; and
(b) an isoamylase comprising an amino acid sequence having at least 85% sequence identity to SEQ ID NO: 242 or SEQ ID NO: 243.

7. The composition of claim 1, comprising:
(a) a Lysin A comprising an amino acid sequence having at least 85% sequence identity to any one of SEQ ID NOs: 1-182; and
(b) an α-amylase comprising an amino acid sequence having at least 85% sequence identity to any one of SEQ ID NOs: 393-445.

8. The composition of claim 7, comprising:
(a) a Lysin A comprising an amino acid sequence having at least 85% sequence identity to SEQ ID NO: 1 or SEQ ID NO: 2; and
(b) an α-amylase comprising an amino acid sequence having at least 85% sequence identity to any one of SEQ ID NOS: 393-398.

9. The composition of claim 1, comprising:
(a) a Lysin B comprising an amino acid sequence having at least 85% sequence identity to any one of SEQ ID NOs: 183-241; and
(b) an isoamylase comprising an amino acid sequence having at least 85% sequence identity to any one of SEQ ID NOs: 242-392.

10. The composition of claim 9, comprising:
(a) a Lysin B comprising an amino acid sequence having at least 85% sequence identity to SEQ ID NO: 183 or SEQ ID NO: 184; and
(b) an isoamylase comprising an amino acid sequence having at least 85% sequence identity to SEQ ID NO: 242 or SEQ ID NO: 243.

11. The composition of claim 1, comprising:
(a) a Lysin B comprising an amino acid sequence having at least 85% sequence identity to any one of SEQ ID NOs: 183-241; and
(b) an α-amylase comprising an amino acid sequence having at least 85% sequence identity to any one of SEQ ID NOs: 393-445.

12. The composition of claim 11, comprising:
(a) a Lysin B comprising an amino acid sequence having at least 85% sequence identity to SEQ ID NO: 183 or SEQ ID NO: 184; and
(b) an α-amylase comprising an amino acid sequence having at least 85% sequence identity to any one of SEQ ID NOS: 393-398.

13. The composition of claim 1, comprising:
(a) an isoamylase comprising an amino acid sequence having at least 85% sequence identity to any one of SEQ ID NOs: 242-392; and
(b) an α-amylase comprising an amino acid sequence having at least 85% sequence identity to any one of SEQ ID NOS: 393-445.

14. The composition of claim 13, comprising:
(a) an isoamylase comprising an amino acid sequence having at least 85% sequence identity to SEQ ID NO: 242 or SEQ ID NO: 243; and
(b) an α-amylase comprising an amino acid sequence having at least 85% sequence identity to any one of SEQ ID NOs: 393-398.

15. The composition of claim 1, comprising three or more of:
(a) a Lysin A comprising an amino acid sequence having at least 85% sequence identity to any one of SEQ ID NOS: 1-182;
(b) a Lysin B comprising an amino acid sequence having at least 85% sequence identity to any one of SEQ ID NOs: 183-241;
(c) an isoamylase comprising an amino acid sequence having at least 85% sequence identity to any one of SEQ ID NOs: 242-392; and
(d) an α-amylase comprising an amino acid sequence having at least 85% sequence identity to any one of SEQ ID NOs: 393-445.

16. The composition of claim 15, comprising three or more of:
(a) a Lysin A comprising an amino acid sequence having at least 85% sequence identity to SEQ ID NO: 1 or SEQ ID NO: 2;
(b) a Lysin B comprising an amino acid sequence having at least 85% sequence identity to SEQ ID NO: 183 or SEQ ID NO: 184;
(c) an isoamylase comprising an amino acid sequence having at least 85% sequence identity to SEQ ID NO: 242 or SEQ ID NO: 243; and
(d) an α-amylase comprising an amino acid sequence having at least 85% sequence identity to any one of SEQ ID NOS: 393-398.

17. The composition of claim 15, comprising:
(a) a Lysin A comprising an amino acid sequence having at least 85% sequence identity to any one of SEQ ID NOs: 1-182;
(b) a Lysin B comprising an amino acid sequence having at least 85% sequence identity to any one of SEQ ID NOs: 183-241; and
(c) an isoamylase comprising an amino acid sequence having at least 85% sequence identity to any one of SEQ ID NOS: 242-392.

18. The composition of claim 17, comprising:
(a) a Lysin A comprising an amino acid sequence having at least 85% sequence identity to SEQ ID NO: 1 or SEQ ID NO: 2;
(b) a Lysin B comprising an amino acid sequence having at least 85% sequence identity to SEQ ID NO: 183 or SEQ ID NO: 184; and
(c) an isoamylase comprising an amino acid sequence having at least 85% sequence identity to SEQ ID NO: 242 or SEQ ID NO: 243.

19. The composition of claim 15, comprising:
(a) a Lysin A comprising an amino acid sequence having at least 85% sequence identity to any one of SEQ ID NOs: 1-182;
(b) a Lysin B comprising an amino acid sequence having at least 85% sequence identity to any one of SEQ ID NOs: 183-241; and
(c) an α-amylase comprising an amino acid sequence having at least 85% sequence identity to any one of SEQ ID NOS: 393-445.

20. The composition of claim 19, comprising:
(a) a Lysin A comprising an amino acid sequence having at least 85% sequence identity to SEQ ID NO: 1 or SEQ ID NO: 2;
(b) a Lysin B comprising an amino acid sequence having at least 85% sequence identity to SEQ ID NO: 183 or SEQ ID NO: 184; and
(c) an α-amylase comprising an amino acid sequence having at least 85% sequence identity to any one of SEQ ID NOs: 393-398.

21. The composition of claim 15, comprising:
(a) a Lysin A comprising an amino acid sequence having at least 85% sequence identity to any one of SEQ ID NOs: 1-182;
(b) an isoamylase comprising an amino acid sequence having at least 85% sequence identity to any one of SEQ ID NOs: 242-392; and
(c) an α-amylase comprising an amino acid sequence having at least 85% sequence identity to any one of SEQ ID NOS: 393-445.

22. The composition of claim 21, comprising:
(a) a Lysin A comprising an amino acid sequence having at least 85% sequence identity to SEQ ID NO: 1 or SEQ ID NO: 2;
(b) an isoamylase comprising an amino acid sequence having at least 85% sequence identity to SEQ ID NO: 242 or SEQ ID NO: 243; and
(c) an α-amylase comprising an amino acid sequence having at least 85% sequence identity to any one of SEQ ID NOS: 393-398.

23. The composition of claim 15, comprising:
(a) a Lysin B comprising an amino acid sequence having at least 85% sequence identity to any one of SEQ ID NOs: 183-241;
(b) an isoamylase comprising an amino acid sequence having at least 85% sequence identity to any one of SEQ ID NOs: 242-392; and
(c) an α-amylase comprising an amino acid sequence having at least 85% sequence identity to any one of SEQ ID NOs: 393-445.

24. The composition of claim 23, comprising:
(a) a Lysin B comprising an amino acid sequence having at least 85% sequence identity to SEQ ID NO: 183 or SEQ ID NO: 184;
(b) an isoamylase comprising an amino acid sequence having at least 85% sequence identity to SEQ ID NO: 242 or SEQ ID NO: 243; and
(c) an α-amylase comprising an amino acid sequence having at least 85% sequence identity to any one of SEQ ID NOs: 393-398.

25. The composition of claim 1, comprising all four of:
(a) a Lysin A comprising an amino acid sequence having at least 85% sequence identity to any one of SEQ ID NOS: 1-182;
(b) a Lysin B comprising an amino acid sequence having at least 85% sequence identity to any one of SEQ ID NOs: 183-241;
(c) an isoamylase comprising an amino acid sequence having at least 85% sequence identity to any one of SEQ ID NOs: 242-392; and
(d) an α-amylase comprising an amino acid sequence having at least 85% sequence identity to any one of SEQ ID NOs: 393-445.

26. The composition of claim 25, comprising:
(a) a Lysin A comprising an amino acid sequence having at least 85% sequence identity to SEQ ID NO: 1 or SEQ ID NO: 2;
(b) a Lysin B comprising an amino acid sequence having at least 85% sequence identity to SEQ ID NO: 183 or SEQ ID NO: 184;
(c) an isoamylase comprising an amino acid sequence having at least 85% sequence identity to SEQ ID NO: 242 or SEQ ID NO: 243; and
(d) an α-amylase comprising an amino acid sequence having at least 85% sequence identity to any one of SEQ ID NOS: 393-398.

27. The composition of any one of claims 1 to 26, wherein:
(a) the Lysin A comprises an amino acid sequence having at least 90%, 95%, 97%, or 99% sequence identity to SEQ ID NO: 1 or SEQ ID NO: 2;
(b) the Lysin B comprises an amino acid sequence having at least 90%, 95%, 97%, or 99% sequence identity to SEQ ID NO: 183 or SEQ ID NO: 184;
(c) the isoamylase comprises an amino acid sequence having at least 90%, 95%, 97%, or 99% sequence identity to SEQ ID NO: 242 or SEQ ID NO: 243; and/or
(d) the α-amylase comprises an amino acid sequence having at least 90%, 95%, 97%, or 99% sequence identity to any one of SEQ ID NOs: 393-398.

28. The composition of claim 27, wherein:
(a) the Lysin A comprises the amino acid sequence of SEQ ID NO: 1 or SEQ ID NO: 2;
(b) the Lysin B comprises the amino acid sequence of SEQ ID NO: 183 or SEQ ID NO: 184;
(c) the isoamylase comprises the amino acid sequence of SEQ ID NO: 242 or SEQ ID NO: 243; and/or
(d) the α-amylase comprises the amino acid sequence of any one of SEQ ID NOs: 393-398.

29. The composition of any one of claims 1 to 28, wherein the supramolecular structure comprises a Z-average mean particle diameter of from 75 nm to 750 nm.

30. The composition of claim 29, wherein the Z-average mean particle diameter is from 250 nm to 750 nm.

31. The composition of claim 30, wherein the Z-average mean particle diameter is from 75 nm to 250 nm.

32. The composition of any one of claims 1 to 31, wherein the supramolecular structure is a lipid nanoparticle.

33. The composition of any one of claims 1 to 31, wherein the supramolecular structure is a micelle.

34. The composition of any one of claims 1 to 31, wherein the supramolecular structure is a liposome.

35. The composition of claim 34, wherein the liposome is unilamellar.

36. The composition of claim 34, wherein the liposome is multilamellar.

37. The composition of any one of claims 1 to 36, wherein the supramolecular structure comprises polydispersity index of from 0.05 to 0.3.

38. The composition of any one of claims 1 to 37, wherein the supramolecular structure comprises one or more lipids.

39. The composition of claim 38, wherein at least one of the one or more lipids is an ionizable lipid.

40. The composition of claim 38 or 39, wherein the lipid comprises 1,2-dioleoyl-sn-glycero-3-phosphocholine (DOPC), 1,2-dioleoyl-sn-glycero-3-phosphoethanolamine (DOPE), or 1,2-dioleoyl-sn-glycero-3-phospho-L-serine (DOPS).

41. The composition of claim 38, wherein the lipid is a sterol.

42. The composition of claim 41, wherein the sterol is cholesterol.

43. The composition of any one of claims 38 to 42, wherein the composition comprises a mixture of lipids.

44. The composition of claim 43, wherein the mixture of lipids comprises DOPC, DOPE, DOPS, and cholesterol.

45. The composition of claim 44, wherein the DOPC, DOPE, DOPS, and cholesterol are present at a molar ratio of from 1-20:1-20:1-5:1-5.

46. The composition of claim 45, wherein the DOPC, DOPE, DOPS, and cholesterol are present at a molar ratio of from 10:10:3:4.

47. The composition of any one of claims 1 to 46, wherein the composition comprises a concentration of lipids of from 0.03 mg/mL to 10 mg/mL.

48. The composition of claim 47, wherein the composition comprises a concentration of lipids of from 1 mg/ml to 5 mg/mL.

49. The composition of any one of claims 1 to 48, wherein the composition comprises a concentration of Lysin A, Lysin B, isoamylase, and/or α-amylase of from 0.1 mg/mL to 20 mg/mL.

50. The composition of claim 49, wherein the composition comprises a concentration of Lysin A, Lysin B, isoamylase, and/or α-amylase of from 1 mg/ml to 10 mg/mL.

51. The composition of any one of claims 1 to 50, further comprising a targeting moiety.

52. The composition of claim 51, wherein the targeting moiety is an extracellular targeting moiety targeting a professional antigen presenting cell.

53. The composition of claim 52, wherein the professional antigen presenting cell is a macrophage or a dendritic cell.

54. The composition of any one of claims 51 to 53, wherein the targeting moiety comprises phosphatidylserine.

55. The composition of any one of claims 1 to 54, wherein the composition comprises a buffer.

56. The composition of claim 55, wherein the buffer comprises glycine, Tris, sodium citrate, sodium acetate, or MES.

57. The composition of claim 55 or 56, wherein the composition has a pH of 5 to 11.

58. The composition of claim 57, wherein the pH is from 5 to 7.

59. The composition of claim 58, wherein the pH is 5.2.

60. The composition of any one of claims 1 to 59, comprising one or more excipients.

61. The composition of claim 60, wherein the one or more excipients comprises CaCl₂), arginine, NaCl, sodium citrate, MgCl₂, and/or glycerol.

62. The composition of claim 61, comprising 5 mM to 10 mM CaCl₂), 0 to 50 mM arginine, 0 to 200 mM NaCl, 0 to 1 mM sodium citrate, 0 to 1 mM MgCl₂, and/or 10% to 30% glycerol.

63. The composition of claim 62, comprising 7.5 mM CaCl₂), 0.5 mM MgCl₂, 200 mM NaCl, 0.33 mM sodium citrate, and 10% glycerol.

64. A method of treating a bacterial infection comprising administering the composition of any one of claims 1 to 63 to the subject in an amount and for a duration sufficient to treat the bacterial infection.

65. The method of claim 64, wherein the bacterial infection is caused by an actinomycetia bacterium.

66. The method of claim 65, wherein the actinomycetia bacterium is a corynebacteriales or propionibacteriales bacterium.

67. The method of claim 66, wherein the corynebacteriales is a Mycobacterium species.

68. The method of claim 67, wherein the Mycobacterium species is M. tuberculosis, M. leprae, M. lepromatosis, M. avium, M. kansasii, M. fortuitum, M. chelonae, M. marinum, M. intracellulare, M. abscessus, M. chimera, M. boletti, M. fortuitum, M. goodii, or M. masiliense.

69. The method of claim 66, wherein the corynebacteriales is a Nocardia, Corynebacterium, or Rhodococcus.

70. The method of claim 69, wherein:
(a) the Nocardia species is N. brasiliensis, N. cyriacigeorgica, N. farcinica, N. nova, N. asteroids, N. brasiliensis, or N. caviae;
(b) the Corynebacterium species is C. glutamicum or C. diphtheriae; or
(c) the Rhodococcus species is R. fascians or R. equi.

71. The method of claim 66, wherein the actinomycetia is a propionibacteriales.

72. The method of claim 71, where the propionibacteriales is a Cutibacterium species.

73. The method of claim 72, wherein the Cutibacterium species is C. acnes.

74. The method of any one of claims 64 to 73, further comprising administering an antibiotic.

75. The method of claim 74, wherein the antibiotic is a cephalosporin, a carbapenem, a penicillin, an aminoglycoside, a cephalosporin, a rifamycin, a macrolide, or a fluoroquinolone.

76. The method of claim 74, wherein the antibiotic is thiacetazone, sq-109, bedaquiline, delamanid, pyrazinamide, or isoniazid.

77. The method of claim 74, wherein the antibiotic is azithromycin, clarithromycin, ethambutol, rifampin, biapenem, or amikacin.

78. The method of any one of claims 64 to 77, wherein the composition is administered intravenously, orally, or via inhalation.

79. A method of assembly comprising:
(a) providing a sample comprising two or more of a Lysin A, a Lysin B, an isoamylase, and an α-amylase;
(b) mixing the sample with one or more lipids in the presence of an organic solvent to form a liposome comprising the two or more of Lysin A, Lysin B, isoamylase, and α-amylase.

80. The method of claim 79, wherein
(i) the Lysin A comprises an amino acid sequence having at least 85% sequence identity to any one of SEQ ID NOs: 1-182;
(ii) the Lysin B comprises an amino acid sequence having at least 85% sequence identity to any one of SEQ ID NO: 183-241;
(iii) the isoamylase comprises an amino acid sequence having at least 85% sequence identity to SEQ ID NO: 242 or 243; and
(iv) the α-amylase comprises an amino acid sequence having at least 85% sequence identity to any one of SEQ ID NOs: 393-445.

81. The method of claim 79, wherein
(i) the Lysin A comprises an amino acid sequence having at least 85% sequence identity to SEQ ID NO: 1 or 2;
(ii) the Lysin B comprises an amino acid sequence having at least 85% sequence identity to SEQ ID NO: 183 or 184;
(iii) the isoamylase comprises an amino acid sequence having at least 85% sequence identity to SEQ ID NO: 242 or 243; and
(iv) the α-amylase comprises an amino acid sequence having at least 85% sequence identity to any one of SEQ ID NOs: 393-398.

82. The method of any one of claims 79 to 81, wherein the organic solvent comprises ethanol.

83. The method of claim 82, wherein the ethanol is provided at a ratio of 10:1 to 1:1 to an aqueous portion of the sample.

84. The method of claim 83, wherein the ratio is 8:1 to 3:1.

85. The method of claim 82, wherein the ethanol is provided at a ratio of 1:10 to 1:1 to an aqueous portion of the sample.

86. The method of claim 85, wherein the ratio is 1:8 to 1:3.


SEQUENCE LISTING (445 sequences)

SEQ ID NO: 1 - protein, 548 aa, synthetic construct
MEKVLPYDRSIVPQETGYWCGPAATQVVLNSRGLIVPEATLAREIGTTVRGTDYVGLIERILDLRVPDARYTSVYIENDPPTADQRETLWRNLKRSIDAGYGVVMNWVAPPSNKPRGVKGSVSPRYSGGTTYHYVAAMGYDDAGERAVWIADSGFQPQGYWVSFDQCASLIPPKGYAYADPTVAPEAPVDADAQAADALLRLMGGSLPFARYQALLPAVRQCLDECECDTEARIAMWGAQVGHESVGLKYMSELWGPTAAQRGYEGRADLGNTQPGDGYRFRGAGPIQVTGRHNFTVLSQWAHREGLVPTPTYFVDNPDELRGDRYGFVGVVWYWTTQRPMNDAADARDLVRATQYVNGGQNGIDNRRDRYNGALAMGADLLKIVNGGDDFMSALTAAEQREMLDMLRWLAAPGTGELRKKFPSRSELRAVGEGLVDTWAGMDLNQDANIHLVAEYVLAKIGDPGAIERLRKLAATTADDRQGSAALARRILDHLDEGGDASEPEPEAPAREVVCESSGGPCVLVANGGDGSCALAGNDCVLRKGVSK

SEQ ID NO: 2 - protein, 554 aa, synthetic construct
MEKVLPYDRSIVPQETGYWCGPAATQVVLNSRGLIVPEATLAREIGTTVRGTDYVGLIERILDLRVPDARYTSVYIENDPPTADQRETLWRNLKRSIDAGYGVVMNWVAPPSNKPRGVKGSVSPRYSGGTTYHYVAAMGYDDAGERAVWIADSGFQPQGYWVSFDQCASLIPPKGYAYADPTVAPEAPVDADAQAADALLRLMGGSLPFARYQALLPAVRQCLDECECDTEARIAMWGAQVGHESVGLKYMSELWGPTAAQRGYEGRADLGNTQPGDGYRFRGAGPIQVTGRHNFTVLSQWAHREGLVPTPTYFVDNPDELRGDRYGFVGVVWYWTTQRPMNDAADARDLVRATQYVNGGQNGIDNRRDRYNGALAMGADLLKIVNGGDDFMSALTAAEQREMLDMLRWLAAPGTGELRKKFPSRSELRAVGEGLVDTWAGMDLNQDANIHLVAEYVLAKIGDPGAIERLRKLAATTADDRQGSAALARRILDHLDEGGDASEPEPEAPAREVVCESSGGPCVLVANGGDGSCALAGNDCVLRKGVSKHHHHHH

SEQ ID NO: 3 - protein, 316 aa, synthetic construct
MAFVSRYGNWYSENGWRMCDANELDRGAVPGTTLVLPIRKGIANLILKAWVAWFHRNVESLNNARGFSDEGAWTPTNDVANSNHLSGTAVDLNWSDHAFRVSYSGFTQAEINKVREGLRLFEGTIWWGQDWTSPKDPMHFQLNYGEGDARNAAFATKLQNGYLRIWSGGGGVIEPPAGVDDIYAQEGDSGDRVKRLQQFFNDNFKSYSQLEVDGDFGPATKAVVVEFQKRVGVLADGIVGPITLAAMVKHGFKTESGVKVANLPEDWTDRQVLNDIWEQLRGPDGKGWEQLGKNAKGENLSLVDAVAEVKKRLEAA

SEQ ID NO: 4 - protein, 327 aa, synthetic construct
MAFIQKQGYWKSENGWRMCDTAELDYTAVPGTSFKLGVRKGSPNIILKALIWRLDKIEPMITSQIGCYTAENSMANSNHNSATAIDYNWNKHPFQKWGTWPNRAAVDKIVDDFRGIIEFGGDWTSPRDEMHFELHFAEGHAGTEALSKDLANGLWGIWKPGAAPAPTPAPGGGNDGILRIGSEGPEVLKMQRGMNSVFKNYRAMPLLEDGIFGAKTKEAVVEFQQRSLIDVDGEVGPQTKAKLAEYGIVLTGATAPTAPPAIVPQKVWPQTASDRELLEYIAAQLGPGDPSWPIKLGNNEKGDPLTVRDRLGLTAKDIEEIKGRLSQ

SEQ ID NO: 5 - protein, 565 aa, synthetic construct
MSFIRSKFAPRALLTVDQWIAIFVAVADELDMPDKRGAVICAAMCAFQEAGADLDDGRGRQIWIPGNMADPCYAADPDAYPHDSEGNDGQSTGPFQQQMNRPGTTPWGWGGNYGDCTGTRKRMDPWDSTRMFFGWPGSGLRDKGYDASSAQAANDSIQRVQGSGVPWAYGQHWGLAQAAYARYLGNPIPAPPSPGGGGPAPALAPNPDWRGDPLYLPQLLRAFGVSVTTYTDADGIPWDQRGHGDFGRISWVLWHHTGSVNETDNGIAHHPALGLAANMLIHPDGHVVLTGSGIAWHGGAGIYPGIPEDNINGISIGIECSYGPDRNGQYTLPWPTAQMNAMIAVGGAISWFLGDTLPPSHQIAHKEWAGRDNPLGINKQGKPDPGNLDMTWFRAQIAARATAGPTDQGDDWMSNPDALQMLADIHRETVTQKSPSRAFVAEDGKQIDTPLGILWNVDGNAWTLVLTEGYWNDVPLAVAVVEDIAANGVRQTSWAGSADKDSDVKFNQWLRDFGQAYCQGLVRRKAQWNAMSAAIIALSAAQAATPKAAPRKRAAKKATATKADQ

SEQ ID NO: 6 - protein, 545 aa, synthetic construct
MIIYRANVEAAKGLVIPRLGNKYVYGGMISPTNLQQGTDCSGIWNDVLGMTVGRFQWGREGEGATTESYRPKTMGGPIPIGGVGPFGTIVVARPQDIPANAVAKLAFHHGPGGGANSHMWGELDGMRIESASSKGLVTAPAAWPIDHSYANAWAYLPGPIVEDGTDVTGIEPQDTLYADVSEWQVPVTDAYANAGYRVLCIRSNDGTYRDKNWATNYAWCKRAVDDGRLKFFIVYFVWRPNWQAAVDTLMSQVGQPHPRMAVMIDVESWGGQIGGDQSNGINAAYERIAGWLGDRRRVIGYGNVGDLDRLWPRKPEGVRLVVAGYGRLPTYPGLIAHQYTDGQGYGGGLPEGAPPFGNCDMNAANGLTATAFAAALGIDTQEDDSLSALTAAEQRELLDAVRETRFLAKILADKRFVSRSPLRHLGEGATETVAGFGLNTDGNLHVLLITELARLGEPGALALLNEVAGADPVKFPDRQGDRQLAQRILASLGKADAQPEPAPTQPQRKVVCEQGGGSCILVANGGDGSCGLGGDECVIRKGVSA

SEQ ID NO: 7 - protein, 403 aa, synthetic construct
MALGAPMENGWPECDLSDCDYATVPGTPLRLPFRKGHPFIILQAFLRDLNEFVEPLMNARGATDEGSWTEDNSVYTSNHKGATAFDYNWSDHPMGNALAGWHGSVLISGEQEPAVRELLRYYTYRGIQLVWWGNDWYSPKDSMHFQMGYDTVNNPAIVQEFIDKFIRPDGYSTYRRGDSSVPAELTVPLTQLSNGRWTSPSAAWAHLITRESGGNPTIIQQIHDVNSGGNEAEGLFQITPKTWKAHNGTEFAPSARFATPQQQGIVAARIITRNPSGSDWGAGLPGREDAKQLLAGLVPTQTTIDPWEELMALEVESFSIYANPGEPKIPVHVMIQSLDAHGPHEPYVEKQARQGDRDAIFRVARTAAGKGKYGNAPGPVKQASQVLKELEEAGVLAEYLKGN

SEQ ID NO: 8 - protein, 540 aa, synthetic construct
MAVARANVEATKTFIRARLGNPYVYGGALSPTNVKQGTDCSEIWQTTLEMTLGRYVPGRQGEGATTESYRPKSMGGPIPDGGIGPFGTIVTRDRNAVPANAVAKIGLHHGPGGGANSHMWGELDGMGIESRGGGVGVITNPRSMRWDDSYANAFAYLPGPITEDGTPAAPEYVLLGRNYENSGDRVRQLQRALNSYGYGLDEDGEYGPLTEQAVGAFQRSKGLEVDGIAGPVTLAALGLTFGRPVESSPEGLALFRQIMSRTDATNAWLATCLPYYIEAMRAAEINTPLRAAAFASQIGHETSGLKYMAEIQTNGPGWTEDRRIYRGRGAIQLTWSSNYRRFGQWCREQGHVTDPELFVKNPELVEHPQWGFLAASWYWRFGGPKPGQINSYADKGDILAVSRCVNGWIEGVDPVGWADRRSRYLNCMALGDRIMNLTTVDPLEELLKMKLNSMSIYRTPGEGPIDAAIMLAALDAHGPHEDYVEKMARRGDEDALFRVARVAMGKGAVTTPAAIKQATDVLLEIAATNRAAVNEALGKV

SEQ ID NO: 9 - protein, 493 aa, synthetic construct
MARRLFRGRAFSENGWPYVDQGSCTWVRIPGAEHVSLQIQNGPPLQVLRAFAADFHAHVEPLRDPDSACWTQDNTVDTSNHPGGTAMDLNWQGADGKTFRYGISEERAYPSPKRERLRELLDFYEGVVFCGGFWSIQDWMHFQMGAGTYDSKADRPTEKTLDFIRRKIRPDGFSTFKRGGGGSAAPDAASVLARAADIPLAKAQEILPTFREGAVLAECTTVPRLAMFIAQTCWESDRYRATEEYANGPMHEERWIYKGRTWIQLTWRSAYEGFGRWCHARGLVDDPMVFVNNPRSLADLKWAGLGAAYYWVTTRRESRKYPTLNEASDARDVLVATQIVNGGTTHLAERTAIYNRAIALGDELLHILGEEDELANPEIEKMIREVHACLFNRIVSQSIYRFPKNPDGSEHPGNIWQLHELIKNGDGMAHMKYVEDSARGGDLTELDRIAVVAAGRGAVRDPWAVQRAQRVLADIERTNPEVIRRYLAQKGAA

SEQ ID NO: 10 - protein, 455 aa, synthetic construct
MPLLTPEQIAATIIKVGNEMGVSPKGQKMAIACVKVESNFLMWANAKSKLSQQFPYDRIGNDSLSEGYYQQQPPWWGGADYEGTRKRMDLVESTRMFFESLKKQRIGTQDYNTDATTPGLWVYMVQRCAPEYKHRYDQEWNAAVALYNKVNAGGSPDAPAPFFPEHNIIDGKGASNRNGQRPRLFVLHTEEGNMVGADLDRWMDEMGDRSYHYAISNDEAWDLVDTDYASWSVLDANRQTINLVFTPSYAAWTRNDWLAKMGNGIKIAAYLAAQDCRKYGIPPVVRVGNSATGYPSLKTNDGITDHYGITVGLKIGNHTDVGGGFPWDVFNTYLQAFYAGSYEEDDMFTDSDRALLQRVHFELTNRWESRSIYREPGEGPVDTLVGMLLNDDGMEHAELVERLAVLGDFDSIRRVRRTAAGKGAVTDKATVARAKKVMSQIPEDVLEEYMKEADK

SEQ ID NO: 11 - protein, 457 aa, synthetic construct
MTVIITRQRAQEVHDRARARKGLPYAYGGAFTNDPRRSTDCSGLVMQTAAWYMGRTDWVGNRYGSTESFRLDHKIVYDLGFKRLPPGGVAALGFTPVMLVGLQHGGGGMYSHTACTLMTMDIPGGPVKVSKRGVDWESHGNRNGVGVDLYDGARAWNDPLFHDFWYLDAKLEDAPAPEDDAVEILSQATGLSTERALEILPAVRDGLIASECVNANRIAMWLAQVGHESASFKYTEEIAKNGRYAPYIGRTWIQITWDYNYRAFSQWCFDRGLVSTPDYFVRNYTDLALLKWAGLGAAWYWTVARTDINALSDRRDLETVTLRINGGHNGIADRRERYDRASALGDRLLALISDAQPIDPFEEEMMREVESFSIYATPGEPKIPLFVMLQSLDAHGPHEPYVEEQARQGDRDAISRVIRTAAGKGKYGTASGPVNQASRVLAELEDSGVLSNYLKGN

SEQ ID NO: 12 - protein, 967 aa, synthetic construct
MTTPAVPTGNVSPPGPTPGTGYSQTSPDQDMQADTYYLTDVGGKEPPPMETLVYTPRVRIIIARDNKQYDVSEDVVAVSINRVENSVSSAAFRLINKPVDQYDENERQGRYTPLFAPMDRVAIFMKRTEWVQVFSGYLDSVALAQLYPGTVDFKASCTLKKLLHTWWDPGLPDSAKIFDQFANDVNEADGQESQLDKGLGSTLRRLLIEVGNMDSSQIHIQRFPMGYLNFMRQQLEKLKTSDATVQEFRRMLLGDDTSGGVGAAAGRQLGVTKGAYNLDQAGRKLEVIRAVDEMGMGVDVRSLGLGQGTNTAAQGGADYQDQEAWKAFAELGRNFQDAALKSDAAVHCFMTIAAESSWVMYANTGVPDSLNFPHEALGHDHDSVGLYQQRQSWGTIDQRMNARESTGMFLNELRKHEWRNMPRPAACQAVQRSAFSDGSNYAVHEAVAIEEVRALRSASGTGQGTGAQSGGATGIPSIGTAPPPVTAGVPGSTGIPGIPTANGQVSSQGVNQTLNRPQYDTAEAIQFGMLQLGKPYAWGAKGPDSYDCSGFTRACYRYIGMNIGDDTYSQVASGTQITMAQAKPGDLIFPAGHGHVVMYLGGNQVIHSPQTGDVVKVSPLWFTPATVITYPGTGYSGPSPLPYDPVRGAQASNGGSDGGVVPGTVAQGTNGGTFQTGSSEPIARNLFSYMFEPGRFQSGISALFGNDEGTTEKAFINDEPLIQSVMSIARAGLRNVQSMPDGSFAAYYPDYFGLDGKDAILDLEDIEMKNVNINWNDDALATHVYVAGTANPTGGSMGLPGWLMTKGVATVENEWLFRRMTSAAPSVKGAEMRNGKEIMKRFGMRPLVQDMPNLFNGEMEFLMALQIFMTKWAEQYSTNVEFTFMPELYPGMRINLVGHNLQVYVAAVTHSGDWENGFTTSATIMAPSNPNIHRIASQVDKDLGFGTHWDQRDIDGKPMPDDYKWMY

SEQ ID NO: 13 - protein, 530 aa, synthetic construct
MALITENGWPQVPRSLCDNLLVPGTTKVRPELRKDDVTIILVAWACWFDRNVRNIEPPDGHRNWWAWSATNDVWNSNHLSGTALDLCADELPWQRKTMPQNQVEITNRGIALFEGTVFWGRNWSRTDEMHFQVGLPPTSPKIREFADRLRNGYLNIFGPADPNAFPLPLGYYYGPLDGPVESISGEYESDSQAAKDGLGRWQAALGLPVTKKWNDGLTPRAAYVLQRSKGWLPNPLFGYGGVYAGEWDAVIREGWRLPANTNLHDVEIPDFEYPLTKWGDYSQYQAATVDDSYPYEVISFRASIANQIDTKWLANMKAAQTLVRQGKLKKIIAYHFWVPGADNWGTFRQAIELAGGVTKELCFMIDVEDGGTKWNIRGDQTAGVKSFIASGQTYFQNPQAASIYWNPTANPDLLVGINDRELRGVKLIVPRYAGPDKAPWTPDGVQWFGHQYSDRENTPPFGPTDINQAKMPLSIFLAAWGANGGPTPDPDDGAGVPSVPVPPVDTDSGVVIPDWDDERVLVAIGAQFDA

SEQ ID NO: 14 - protein, 504 aa, synthetic construct
MARRLFRGRQFSENGWPYVDQGSCTWDEVVPGVWLQIQNGIPFTVLRAFARDFNEHVEKLRDADSACWTQDNSVDTSNHPGGTGADFNWNGEDGRTFRYGITEARAYPGDKARRLRELLDFYEDNVFCGGFWDIRDWMHFQMGYGTYDSKADRPTQKVTDFVARKIRPDGWSTFKRGGGGAPAPAADAAAILARATGVTLDKAREILPTLRDGLILSECTTVPRIAMWLAQCGHESAHFNATEEYQNGPMDQERWIYKGRTWIQLTWRSAYEGFGRWCHARGLVSDPTVFVNNPRSLADLKWAGLGAAYYWTTTVRSTRKYPTLNQASDARDVLVATQIINGGTNGLEDRQNRYNRAIALGDELLQIIGEEDFLSALTPAEQRELLDLLRWGFAPEYGEFRKLFQNEDMYREDNVRRRTAVGAALDSRTFSWEDRVEKSAERGELWAIDLVVRAARGTLPGVIRPGGNTPDPFLVNHARQVLADVERINPEALKKYLALEGGSR

SEQ ID NO: 15 - protein, 517 aa, synthetic construct
MTERVLPFDRKIIRQETGYWCGPASTQVALSARGKYVDEATLARECKTTVNGTDNVGQIERVLDVRLPEGKYTSMYPGGTTIGTPARPAAERKTRFWWDIVRSIDNGFAVILNWVVPPARKPIKAVKGSTNPSYGSGTTYHYVTAVGWSDEGNGGRPAVLIADSGFSPNVYWVDLDTAFALIHTDLWKGYAFADLPLIAPPPPGVEVPPGIPVKIGDPPIAVTPPAPVPPTQPPTVVKITDPFTGAIWSPNRYSPRGLGTPGWIAVHTQEGGRTARDLALFLANPANEVSYHSVNDDVEVLKVVAEGDASWSASNANKYAFHHCFAGSYAGWSRDKWLSPDASDGKNEDVQLTKGAHVVAWWCDKYGIPAEWIGGRAQPPWGARGILGHVDLGQWGGGHFDPGGNFPVNEFIRRVVKFLTGEDQPPLVPLPPVVVPGTNPDAYSDWMLVRGDPRNDVDRVMRVQRRLKNAYKGYAGHLAVDGDFGPATQAAVREFQRRSNLVADGIVGPMTAAALRP

SEQ ID NO: 16 - protein, 557 aa, synthetic construct
MTEKVLPYDRSIVPQETGYWCGPAATQIVLNTRGLIVPEATLAREIGTTVRGTDYVGLIERILDLRVPDARYTSVYIENDPPSAAQRETLWRNLKRSIDAGYGVVMNWVAPPSNYPRGVKNSASPRYGGGTVYHYVAAMGYDDDPAARAVWIADSGFQPQGYWISFDQCASLIPPKGYAYADPTVAPEAPIDADAQAADALLRLMGGSLPFARYQALLPAVRQCLDECDCDTEPRIAMWGAQVGHESVGLKYMSELWGPTAAQQGYEGRADLGNTQPGDGYRFRGAGPIQVTGRRNFTVLSQWAHGKGLVPTPTYFVDNPDELRGDRYGFVGVVWYWTTQRPMNDAADARDLVRATQYVNGGQNGIDDRRDRYNGALAMGADLIKILNGGDDFMGALTAAEQREVLDLLRWIAAPGTGELRKRFPSRSPLRHLGEGLVDTAVGVGLNDDANDHVVLVKDLAEIGDPDALALLHEVAGPENTPERYPDRQRDKVLAQRILDSLSKAPQVGDTPIVVNTPARRVVCESSGGPCVLVANGGDGSCALAGNECVLRQGATK

SEQ ID NO: 17 - protein, 493 aa, synthetic construct
MTLIVTRDHAQWVHDMCRARAGNRYGYGGAFTLNPRDTTDCSGLVLQTAAWYGGRKDWIGNRYGSTESFRLDHKIVYDLGFRRLPPGGVAALGFTPVMLVGLQHGGGGRYSHTACTLMTMDIPGGPVKVSQRGVDWESRGEVNGVGVFLYDGARAWNDPLFHDFWYLDAKLEDGPTQSVDAAEILARATGLAYNRAVALLPAVRDGLIQADCTNPNRIAMWLAQIGHESDDFKATAEYASGDAYDTRTDLGNTPEVDGDGRLYKGRSWIMITGKDNYRDFSRWAHGRGLVPTPDYFVVHPLELSELRWAGIGAAWYWTVERPDINALSDRRDLETVTRRINGGLTNLDDRRRRYNLALAVGDQLLTLIGDDDELADPTIQRFIREIHGALFNTVVTQSPYGDPQNPDGSEPRSNLWQLHELIKNGDGMGHARYVEESARAGDLRELERVVRAAKGLGRDRSPEFIARARNVLAQIEAANPEYLQAYIARNGAL

SEQ ID NO: 18 - protein, 504 aa, synthetic construct
MALGGRMENGWPECDLSDCDFATVPGTPLRLPFRKGHPFIILQAFLRDLNEFIEPLMNSRGATDEGSWTDNNSVYTSNHKGASAFDYNWDDHPMGPAAPDPRAGWNGSVLIDGDQTPAVRELLSFYTYKGIQLVWWGNDWDSPKDSMHFQMGYNTVNNPAIVQEFIDKYIRPDGFSTFRRGGNAPQAPVVDAATVLAKAVGIGYEKAKTILPAVRAGLIQSECKSSPRIAMWLAQVGHESDSFEATEEYADGNEAEERWKYKGRTWIQLTWLSAYQGFGRWCFDRGLVSDPNCFVNDPRSLAELRWAGLGAAYYWTTTVRSTRKYPTLNQASDARDVLVATQIVNGGTNGLDKRQARYNRAIALGDELLQILGGDNDDMAQVPQDQWSWVLKAVSELHGAMFNRIVSQSPYRYPKNPDGSEPPGNVWQLHELIKNGDGMAHMAYVENAARGGDLTELGRVVRAARGQGAETAPWFVQRAQRVIAEIEKTNPDLLKRYIAERGVA

SEQ ID NO: 19 - protein, 389 aa, synthetic construct
MTTKDQVAQITIAEAKARGYTRSECLAVMSTFYQESGWNDTIWDPTHTTYGIAQQDGSYPHRFDGAAAQIKGFFDKLDVWRAKPGASTDIWLNICWMQQAPNWPSADYWYANGRRAYLTEIKSRIATVTPYLDKYWPTTGGTDVPETRPPYNEFPIWSENHYNTKRTVNDIDAFLLHTSEGFVGRDDAAEALSLWYQPRSRQVAYHYAVSQASDGGVTVVDNVDTDYASWSALSANGRSINLCFAGTRAAWSRNEWLGKFGNAIDVAAYLAVQDCKKYNIPTKVIAPPYTGRLPGITDHRYVTQILKDGTHTDVGDGFPWDYFTERVNHWANGGKTEPEPPKVKRFPDDWTDREILVEILRQLRGYNLTGWPQLGGKTLVDAVAELLGH

SEQ ID NO: 20 - protein, 449 aa, synthetic construct
MATRFMPMKAGTYSIASGFGPRWGTQHRGLDFAAADGTPIYAAQAGTVAHIGSAQGFGQWIVVDHPAEDGAGTTVYGHMWNAFATGLKLGSHVKAGQLIGFVGSNGQSTGPHLHFEVHPTVWAAGSQIDPAPWLRGALDPGGDPPPAIVKPALPPAPAPPPGGNTVGDPVWLPEALRPAVSNLVEYPGWRNRGHGDFKDIRGVMVHHTGGPASARSIADGRPDLAGPLSQLHIARDGTVTIIAAGVAWHAGSGSYPWLPANMGNWHLIGIECEWPYRGGIGERNAHEEPWRREQILAIRNTCAALLQWLRFGVDRLIGHKDYAGRAQGKWDPGNMSMPWLRGEVEKDMSGFAFPGEDGPRPIIVDSGLPPVPLPPAPAPAVAGVLLHRGMNGPAVRRLQEVLRRWYSKLAVDGDFGPATEAAVRDFQRLRPPLDADGVVGPATAQRLGL

SEQ ID NO: 21 - protein, 472 aa, synthetic construct
MAVTRANVEATKNFIRARVGNPYVYGGALSPTNVRQGTDCSEVWQTVLEMVHGRYNPGRQSEGATTESYRYIPVGGVGPFGTIRVNHWRDIPANAAAKLAFHHGPGGGANSHMWGELDGMLIESAGSKGLVTNGRAMTIDNSYATAWAYLPGPILEDGSPIPEDPNAVTWGIDISNHQGEMDLNRVKAEGFDFIWCKVSEGANYRDPFWPGNRDRARAAGLILAGYHYVRTGDPAAQAKTFVEHLGDKSIPAMLDFEDGSGNIEQFWAVKAEIEKLGVQVRLSYIPDWYHERIGKPDLSKVPGLIASEYVSGTGYASVLYPGNNSNFWKAYGGRTPDVLQFTDRALVAGKSVDANAFRGTPDKLRRLLGAGGDDFLSALSDAEQRLLFDRTNQLWGAVFNDIPSTSKYKAAGEGAIWKSKDLWRNIDGMMHETLVERQAMMGNPEALALVKREADKGDKWAACVYKYCTEEA

SEQ ID NO: 22 - protein, 570 aa, synthetic construct
MSFIRSKFAPRALLTVDQWIAIFAAVADELDMPDKRGAVICAAMCAFQEAGADLEDGNGRQIWIPGNMADPCYADDPDAYPHDSEGDDGQSTGPFQQQMNEPGKAPWGWGGNYGDCAGTRKRMDPWDSTRMFFGWPGSGLKAKGYDASSAQAANDSIQRVQGSGVPWAYAQWWGLANAAYDRYLGNPIPAPPSPGGGSGPAPALVPNPAWRGDPLFLPQVLRAFGVNVSTYTDADGIRWDQRGHGDFGKISWVLWHHTGSVNETDEGIAHHPALGLAANMLIHPDGHVVLTGSGIAWHGGIGIYPGIPEDGINQVSIGIECSYGPDENGQYTLPWPTAQMNAMIAVGAAISWFLGDTLPVDHQISHKAWAGADNPLGINKQGKPDPGNLDMNWFRAQIAARTALGPTTGGDDWMADPTALELLRDIHRETVTQKSPSRSFMAEDGKLIDSPLGIEWNTDGNAWTLVLTEGYWNDVPLAIAVVEDIAANGVRQTSWAGSADKDSAVKFNQWLRDFGQAYCQGLVRRKAQWNALVAAVASLSAAQAAGATTAVKAAPRKRAAKKAPAPKDAE

SEQ ID NO: 23 - protein, 489 aa, synthetic construct
MITENGWPDCGPNDLDLKPIPGTDVVIPLQKGQPSLILKAFAADLNWYVESVYNSRGGTDEGGWTGTNKVPTSNHLGGTAFDYNWSDHPLGYAAPDPRAGWNGSSIIKGDQTPYVRELLDYYEGMVYWGNDWRSPKDSMHFQMGYNTYGPAGSTTWKRVEDFIARKIDITTGFSRFKEHKGTTGVNFTALLSEAMLDVSGVDYEYYVPLVARTLRDCECTTEARIAMWCTQVGHESLSLKYMEEIADGSKYEGRTDLGNTQPGDGKRFKGRGPIQVTGRYNYTKFSEWAHSKGIVSTPDYFVMNPSELSKPEYGFHAVTWYWTVQRPMNQAADARNLEQATKYVNGGLNGLPDRKMRYDRTTAMGPRLLNLLLGEEDWMSDPTIVKMIKEIHGCLFNQIGSQSRYRADGEGDIWKLHELIKNDDGMVHEAHIERLAVLGDPSSIALVARNSERGDKLAQAVLIRIEDKYLTETQQHLKNTIIAQRFPEG

SEQ ID NO: 24 - protein, 311 aa, synthetic construct
MPILRANVDYFWSIARPRNRKPYGYGGVWVKNDLNRTTDCSGIVTHALDAVINGPNMTWSRHGISTESYRYVGGPGSRGPFGTIRVARPQDIPADAAVKIGLMHGPGGGANSHMACTIEGVAIESRGGTVASGGGQWVGGNGRHYNNPLFHDWFYLPGPIVGSGASTPAQPQAPGAVYLGRDCSRYECTGERVKALQARLNRDYPAYSDLDEDGEFGPLTEAVVKQFQLRSALTADGIAGPATLAALGLSFQAQEVPPVTAPKPVIVGPADDQLTMRWNALGGQTLVEAVAQIRDKVCGTEDRSKPGVVTK

SEQ ID NO: 25 - protein, 498 aa, synthetic construct
MALGGRMENGWPECDLSDCDYATVPGTPLRLPFRKGHPFIILQAFLRDLNEFIEPLMNARGATDEGSWTDNNSVYTSNHKGATAFDYNWSDHPMGNANAGWNGSVLIAGDQVPAVRELLRFYTYRGIQLVWWGNDWNSPKDSMHFQMGYNTVNNPQIVQEFIDKFIRPDGFSTFRRGGAAPSAPAVDAATILARATGIAYAKAQEILPTLRDGLILADCKSVPRIAMFVAQTCWESDHYNATEEYANGPLNEERWIYKGRTWIQLTWKSAYAGFGQWCFERGLVNDPNIFVNHPRMLAETRWAGLGAAYYWLTTRRPTRKYPTLNQASDARDVLVATQIINGGTTHLAERQALYNRAIALGDDLLLILGGDEDEMAGWDQTKVDRAMVLLENIAGVRRKSRSPLRWPYEKEIDTAIGFAWTADANVHVQLVEKLAVIYGDPTSIALLTAVANTDEPGREGDRELARRILARCSEDAVQEAHKRINEWLEAEKAHKVGV

SEQ ID NO: 26 - protein, 518 aa, synthetic construct
MVTRANVEATKEFIRARLGNPYVYGGALSNNVRQGTDCSEVWQTVLEMAHGRWVPGRQAEGATTESYRGIKPGQVGPFGTIAVAHWRDIPANAAARIAFHHGPGGGANSHMWGELDGMRIESGGSKGLVTGDRALAVESTYATNWAYLPGPIGGTASLGIGSTGPAVITLQAKLNTRGAGLEVDGEFGPLTAAAVTRAQQELHVVGDPPGIAGEATLRALGILNDNGAIPANPDTLAAEYLAAATGLSVARAQQILPAARDGLLRSDATNVNRIAMWLAQIGHESAGFNATEEYEKGDGGATERWKYLGRTWIQLTWLSNYLGFSKWCFERGLVPTPTYFGDNPRELADLKWAGLGAAYYWTVARPDINALSDRRDLDTVTRRINGGTNGIEDRRNRYNRALAVGDNLLRIIANSPEPQDEWEALMADQTRYSSRAWFRSDDVANLTPLDLLRNVDAMKYDERVIQAAFRGQVWAINAIATLAKGEGPEGKNPAAVLEAQDIIRQLQAALAAQNGASK

SEQ ID NO: 27 - protein, 493 aa, synthetic construct
MTLIVTREHAQWVHDMCRARAGNRYGYGGAFTLNPRDTTDCSGLVLQTAAWYVGRKDWIGNRYGSTESFRLDHKIVYDLGFKRLPPGGVAALGFTPVMLVGLQHGGGGVYSHTACTLMTMDIPGGPVKVSQRGVDWESRGEVNGVGVFLYDGARAWNDPLFHDFWYLDAKLEDGPTQSVDAAEILARATGLAYNRAVALLPAVRDGLIQADCTNPNRIAMWLAQIGHESDDFRATAEYASGDAYDTRTDLGNTPEVDGDGRLYKGRSWIMITGKDNYRDFSRWAHGKGLVPTPDYFVVHPLELSELRWAGIGAAWYWTVERPDINALSDRRDLETVTRRINGGLTNLDDRRRRYSLALAVGDQLLTLIGDDDELADPTIQRFIREIHGALFNTVVTQSPYGDPKNPDGSEPPSNLWQLHELIKNGDGMGHARYVEDSARAGDLRELARVVRAAKGLGRDRSPEFIARARNVLAQIEAANPEYLEAYIAKNGAL

SEQ ID NO: 28 - protein, 561 aa, synthetic construct
MTEKVLPYDRSIVPQETGYWCGPAATQIVLNSRGLIVPEATLAREIGTTVRGTDYVGLIERILDMRVPDARYTSVYIENDPPRTEQREALWRNLKRSIDAGFGVVMNWVAPPSNYPRGVKGSVSPRYSGGTVYHYVAAMGYDDAAPGVGRAVWIADSGFQPQGYWISFDQCASLIPPKGYAYADVDAPGGPAAPVDADAQAADVLMRLMGGSMPFGYYQEALPAVRQCLVECGCDSELRIAMWGAQVGHESVGLRYMSELWGPTAAQAGYEGRVDDLGNTQPGDGYRFRGAGPIQVTGRRNFTVLSQWAHSKGLVPTPTFFVDNPDELRGLRYGFVGVTWYWTTQRPMNDFADRRDIEGASIAVNGRGANGRANGIDDRINRYNGALAMGADLLKITDGGDDFMSALNAAEQREMLELLRWIAAPGTGELRKKFPSRSELRAIGEGAVDTWAGMDLNQDANIHLVAEYVLAKIGDPGAIERLRKIASTTAADRQGSAALARRILDHVDEPHETPPDSEPAAPVRKVACAQSGGGCVLVANGGDGSCALGGDDCVLRKGVTA

SEQ ID NO: 29 - protein, 565 aa, synthetic construct
MANRIVYGRSNSENGWPMVDAGSCQWVTVPGTNVSLQIREGQPAKILGAFVADINAYVENVTDADSGCWTPTNSVASSNHLSGTACDVSWNRHPFHVRNTYNAAQRATIRELLDWYEDTVFWGGDWNSPIDEMHYQMGYDTYGSQNVARVQDFINRKIRPDGFSTFRRGGSTAPAPAPQPAVDQVQVLANATGLSRGRAAEILPAVVDGLAASSCKNVNRIAMWLAQIGHESDNFNATEEYDKGDGGRTERWKYLGRTWIQLTWASNYAGFSQWCYGRGLVNSPTYFVDRPAELAELKWAGLGAAYYWTVARPQINSLCDARDLLAVTKAINGGTNGLPDRQNRYNRALAVGDALLALATSTSTPTQEDDMAQVPQEQWDRVYRELTQRLPSRSPLRHLGEGPVDTLAGFVLNADGSEHVEIVRLLAGYGHPPTLALLREVAGADPVRYPDRQEDRKLAQAILAEVTATPAAPAATVASVAPTEPQIVYLPAPTPEPVAAPPAPTPEPVAAPPAPAAGGSTGQIIGQAYDALEALQLSGVLEAAERAPLSALITVLQTKTQGASA

SEQ ID NO: 30 - protein, 443 aa, synthetic construct
MDDRDMADRFFPMRDGTYTLSSGFGARWGTQHRGLDFAAKDGTPIYAAQAGTVAYIGPAQGFGQWIVIDHPAADGAGTTVYGHMWNAFATGLKAGDRVQAGQLIAYVGANGQSTGPHLHFEVHPTVWRQGSQIDPLPWLHGSRNPGDAPAPAPADPPASAPLGGQRMQDPFTGEVWSPNRSVRQKPAPRWIAIHTQEGGRTARDLCEGWLAKRESQVSYHVAVDDREILKVVAESDRPWAAANANDYAFHVVAAGSYAGWSRGKWLETDASDGKNEDVELTNLAKVCAWWCQKYGIPAEWIGGRGVPWGLDGICGHEDFGAWGGGHHDPGPGFPADELIRRVRALLGGSTPEPLPPAPPVALPGTNPDQYAGVLLYRGRPGQDPRQVRVLQTRLKRAYSKLDVDGIFGPHTEACVRDYQHLHPPLVADGIVGPATAAALGLVF

SEQ ID NO: 31 - protein, 459 aa, synthetic construct
MTAVVTRQRAQQVHDMARARAGLPYAYGGAFTNDPKRSTDCSGLVMQTAAWYMGRTDWVGNRYGSTESFRLDYKIVYDLGFKRLPPGGIAALGFTPVMLVGLQHGGGGMYSHTACTLMTMDYPGGPVKVSKRGVDWESHGNRNGVGVDLYDGARAWNDPLFHDFWYLDAKLEDAPTSGDEAAATLSEATGLSLQRASEILPAVRDGLVASDCTNANRIAMWLAQVGHESVSFNYTEEIAKNGRYAPYIGRTWIQITWDYNYRAFSEWCFERGLVTSPDYFVRNYTDLALLKWAGLGAAWYWTQARGTQINDASDRRDLYTVTQLINGGQNGAADRKARYHRASALGDRLLSLIADAPTTDTFEELLMMEVESFSIYADPGEPKIPLYVMLQSLDAHGPHEPYVEAQARIGHADSIRRVVRTAAGKGKYGTAPGPVNQASRVLAELEASGVLAAYLKGTA

SEQ ID NO: 32 - protein, 451 aa, synthetic construct
MADRYLPMREGTYQIASGFGPRWGTVHRGLDFAAKDGTPIYAAQAGTVVHIGAAQGFGQWIVVDHPAEDGAGTTVYGHMWNAFATGLKQGARVEAGQLIGYVGSNGQSTGPHLHFEVHPTVWAAGSQLDPAPWLRGARNPGDPTAPRPAPAPAPAPAPPPGGNTVGDPIWLPDALRPAVSNLVEYPGWRNRGHGDFKDIRGVMVHHTGGPASARSIADGRPDLRGPLSQLHIARDGTVTIVAAGVAWHAGAGSYPWLPTNMGNWHLIGIECEWPYRGSGISERNAGDEPWRREQIIAIRNTCAALLSWMRFGVDRLIGHKDYAGRAQGKWDPGNMSMPWLRGEVEKDMSGFAFPGEDGPRPIIVDTGLPPVPLPPAPAPAYAGVLIHRGMTGPAVRRLQETLRRWYSKLVVDGDFGPATEAAVRDFQRLRPPLAADGVVGPATATKMGLVL

SEQ ID NO: 33 - protein, 489 aa, synthetic construct
MGMTLENGWPECDLSDTERLTIPGTALSLPIRKGQPHAILQAFFRDVNEFIEPVMNARGLSDEGSWTENNSVYTSNHKGATAVDWNWSDHPVQIRDAGWDGSVLINGSQVPAMRELLAWYEGMVFWGNDWNSFIDSMHFQMGYNTFGAQNFDRVHSFIQRKIRADGFSTYRRGGTPRGGGFAEVPAAPLHPIKPTSGLTPEVLWRIAGGAASKLSVGHFERWFDELVECQAACGVLGNIDRSAMWYSQVFPESGNLVYTEEIASGAAYEGRCEGLGNCQPGDGVRFKGRSFMQVTGRSNYTKMSGWAHGKGYVPTPDYFVVHPDQLDDERFAFLGVTWYWTTQRPMNDAADARNLELATRYVNGGLTNLEGRRAVYNRALAENANLLLTNPVEPWEELMATAVPSLSIYANPGEPDVPLAVMLAALDAHGPHEPYVERQAIEFGDADSIRRIARTANGQGKVKTPAAIKQATDAFRLIPAEFVRAAIPA

SEQ ID NO: 34 - protein, 449 aa, synthetic construct
MADRCMPLADGTYRLGSLFGNRKGGFHRGIDFEADDGTPIYASQAGTVAHIGSASGFGQWIVIDHPAEDGAGTTVYGHMWNAFATGLRAGQRVAAGQLIGYVGSNGESTGPHLHFEVHPTVWAAGSQIDPAPWLRATRNPGGVPAPIPPPAPAPAPAPPPGGNTVGDPVWLPDALRPAVSNLVEYPGWRSRGHGDFKDIRGVMVHHTGGPASARSIADGRPDLAGPLSQLHISRDGVVTIVAAGVAWHAGSGSYPWLPTNMGNWHLIGIECEWPYGEPGINDRNAHTVPWRRPQIIAIRNTCAALLQWMRFGVDRLIGHKDYAGRAQGKWDPGNMSMPWLRGEVEKDMSGFVFPGEDGPRPIIVDSGLPPVPLPPAPAPAYAGVLLHRGMNGPAVRRLQEVLRRWYSKLAVDGDFGPATEAAVRDFQRLRPPLAADGVVGPATAQRLGL

SEQ ID NO: 35 - protein, 706 aa, synthetic construct
MQVLGQEITKVRVAGADAARVRIFGEDAWTAEPPTPVPDPVSYFDWHEGSGSTTTSIVSSHVLTAVNPSAAWDGESAAGQFEGDIGDVATSTWSIAVEFTLSAGSSWRNILHTSGAGAADEVWLQLNGSTLSIWTSAGASTGFDPAQPTLPPNLPIQLVMTNDGTTQRVYVDGELVSSRARNNILEPTSAVIMDTSEKLNGYVHSLRFWDVVLSDVEVEALMPDSGDEEDPPLFRGMTDEITQYGITWTFDQEYMAGQFITGDWWVVGPVTVTSVSPAPQGRLDGEDDDDLTGEAGQRVDDRMRHGSMVLTDVTTDQAYDSRCRFYNPARAISYPYNLAANRSLISSVSAVHPGGSEMGDLADGAEGRVMETAAVLTCLDEEPPEDAFRPCYWGADKSKIVRASDIQWDRLANVTPASSPPTLVEYARFFERPWLDHMQAYWVGQFLWPWKNQPGYGRGYAAVVGTAGLLVNCNYTEAQKRPIVHGLIQLGIDLAGMLKADSCFRVSGGHGSGRKFPVVFAHLMLDDDPYFQFEADAVGADVGEDGDGEPQALFGEDASTYWGETYYGAPACFQMVGYSSEVPPHQESPPPYTDWDDTSEGYRQCCTVGAWVGQTLAILLMGAKAAWNHDSYFQVVDDWMADPDPYAANRGGHPRPDDWQETNAWHPWATRMWNLHRSSVPAQDDGADNYKWMALDREWVPNPPPA

SEQ ID NO: 36 - protein, 963 aa, synthetic construct
MTTPNVPTGNVTPPGPQPGTGYSQTTPDQEQGVDNYYLTDVGGKEPPPMETLVYTPRVRIIISRDNKQYDVSEDVVAVSINRVENSVSSAAFRLINKPEDQYDENHRQGRYTPLFAPMDRVAIFMKRTEWVQVFSGYLDSVALAQLYPGTVDFKASCTLKRLLHTWWDPGLPDSAKLFDQFANDVNEADGQESQLDKGLGSTLRRLLIEVGNMDSSQIHIQRFPMGYLNFMRQQLEKLQSSEKSYMEFRRMLLGDDTSGGVGAAAGRQLGVTKGAYNVDQAGRKLEVIRAVDEMGMGPDLRNIGLGQGLGTTSGAGKDLEDQEAWKLQQELGRNYTEAAQKNDAAVHCFMTIMAESSWIMYANTAVPESLNFPHEAVGHDHDSVGLFQQRQTWGTIDQRMNPRESAGMFLNELKKHDWRNMPRPAACQAVQRSAFSDGSNYAVHEAVAMEEVRALRTSGAAPSPGGQQSTISPVGTAPPPITAGVPGSTGIPGIPTVNGQASTQGANQALNRPQYDLAEAVQFGMMQQGKPYQWGAKGPNSYDCSGFTRACYRYIGLNIGDDTYSQVASGTKITMAQAKPGDLLFPAGHGHVVMYLGGNQILHAPQTGDVVKVANLYFTPATVCTYPPATYSGPSPIPYDPTRAAQASNGGSDGGVVPGTAAQGTNGGTYQTGSSEPIARNLFSYMFEPGRFQSGISALFGMDEGQTEKAFINDEPLIQSVMSIARAGLRNVQSMPDGSFAAYYPDYFGLDGKDAILDLEDIEMKNVNINWNDDALATHVYVAGTANPTGGSMGLPGWLMTKGVATVENEWLFRRMTAAAPAVKGAEMRNGKEIMKRFGMRPLVQDMPNLFNGEMEFLMALQIFMTKWAEQYSTQVEFTFMPELYPGMRINLVGHNLQVYVAAVTHSGDWENGFTTSATIMAPSNPNIHRIAQKIDSDLQFGTHWDQRDIDGKPMPSDYSWMY

SEQ ID NO: 37 - protein, 530 aa, synthetic construct
MALITENGWPQVPRSLCDNLLVPGTNRVKPELRKDDVTIILVAWACWFDRNVRNIEPPDGHRNWWAWSATNDVWNSNHLSGTALDLCADELPWQRRTMPQNQVEITNRGIALFEGTVFWGRNWSRVDEMHFQIGLPPSSPKIREFADRLRNGYLNIFGPPDPNAFPLPLGYYYGPLDGPVESISGEYESDSQAAKDGLGRWQAALGLPVTKKWNDGLTPRAAYVLQRTKGWPANPLFGFGGVYKGEWDAVIREGWRLPADVNLHNVVIPEFEYPLTKWGDYSQYQAATVDNTYPYEVISFRASIANQIDEKWLANMKAAQILVRQGKLKKIIAYHFWVPGADNWGTFRRAIEAAGGVTKELCFMLDVEDGGTKWNIRGDQTAGVKSFIASGQTYFQNPQAASIYWNPTANPDLLVGINDRELRGVKLIVPRYNGPDKAPWTPDGVQWFGHQYSDRENTPPFGPTDINQAKMPLSIFLAAWGTNGGVAEPDDPGTPVPAEPVPPVDTDSGEAEPDWSSERVLVAIGAQFGA

SEQ ID NO: 38 - protein, 436 aa, synthetic construct
MTNDVYAREIIRAGKDQGITPRGIVIAFATVFVESNWKNYANSKVPESLALPHDAVGSDGKSVGLFQQQVVWGNGWWWGDAATCMDPYKSAVLFFQRLKKLDYNNTSRSPGSYAQDVQKSAFPDRYGQRMAEAQRYYDRLTGGSAALTPEPGFSGDPWWLAEVLRDEGLRVFEVDGWQNRGQGDQGRFWGVVFHHTGSPSETPEGIAFHPTLGLAAHILIRPNGDVWVCGIGKANHAGKGSWWGVPTDNGNPVLLGVEVAILPREGAPHRSGWPDVQYDATVKVHAALLRKLGLGSDRVISHKEWAQLGPAGWRQGKWDPGAIDMNVFRADVQAQINSKTGDDPLADPDVVKKINEIHACLFNQIPSKSKYRDLNEGNKWQLHQLIGNDDAMIHEMLVERQAMMGNPEALALVKREADKGDKWAAVVYQYCTEAPE

SEQ ID NO: 39 - protein, 430 aa, synthetic construct
MSFTWFRPEGPLRTREQIAREVHAVSLARGLDELATVIALMTISTEVGTGTGDDRKWWCPANDRVPATKNYPHDSRSDDNRSSGYFQQQPGPNGEPWWGTPENMMTLPQAANTFLERLSDDYRRAANNPRLAGEFAQRVQQSAYPDRYADKWDEAWSVLRRALNETTPEEPVTENRPAYNEFPIWSANNSARSGKPTMFLIHTQEGGGGDAAAENLAKWFQNANGVSYHYTISQASDGGVTVVDCVDTDRAAWSVGNANSISINLCFAGSRASWMRDQWMKQSNAIDVAAYLAVQDAKKYGFEPLVVPPPYVNGRPGISDHRWVTDVFKWGTHTDVGDWFPWDYFTERVNHWANGGKTEPEPPKVKRFPDDWTDREILVEILRQLRGYNLTGWPQLGGKTLVDAVADLRTDIIDLQGAIEHGEITLGGAQ

SEQ ID NO: 40 - protein, 540 aa, synthetic construct
MAVARANVEATKTFIRARLGNPYVYGGALSPTNVKQGTDCSEIWQTTLEMTLGRYVPGRQGEGATTESYRPKSMGGPIPDGGIGPFGTIVTRDRNAVPANAVAKIGLHHGPGGGANSHMWGELDGMGIESRGGGVGVITNPRSMRWDDSYANAFAYLPGPITEDGTPAAPEYVLLGRNYENSGDRVRQLQRALNSYGYGLDEDGEYGPLTEQAVGAFQRSKGLEVDGIAGPVTLAALGLTFGRPVESSPEGLALFRQIMSRTDATNAWLATCLPYYIEAMRAAEINTPLRAAAFASQIGHETSGLKDMAEIQTNGPGWTEDRRIYRGRGAIQLTWSSNYRRFGQWCREQGHVTDPELFVKNPELVEHPQWGFLAASWYWRFGGPKPGQINSYADKGDILAVSRCVNGWIDGVDPVGWADRRSRYLNCMALGDRIMNLTTVDPLEELLKMKLNSMSIYRTPGEGPIDAAIMLAALDAHGPHEDYVEKMARRGDEDALFRVARVAMGKGAVTTPAAIKQATDVLLEIAATNRAAVNEALGKV

SEQ ID NO: 41 - protein, 441 aa, synthetic construct
MADRFAPMRLGTYTLASGFGPRWGSMHRGLDFAAKDGTPFYAAQAGTVVYIGPADGFGQWIVVDHPAEDGAGATVYGHMWNAFATGLRVGSRVKAGQLLGFVGSNGQSTGPHLHFEVHPYSWRAGSQIDPSPWLNGAKNPGDPAPAPAPAPAPKPAPAPPPPGGRMLDPFTGAVWSPNRSKRSKGNPRWIAIHTQEGGRTARDLAEGWLAKPASQVSYHAAVDERERLKIVAESDRPWAAANANDYAFHVVAAGSYAGWSRGKWLETDASDGKNEDAELTSLAMVVAWWCEKYAIPAEWIGGRGIPWGRDGICGHADFGQWGGGHHDPGAGFPVDELIRRTRGFLANGSAPAPLPAPAPVIPRGVEPVPGVLLYRGRIGQNPDQVRALQTALRKWYSKLAVDGDFGPATEAAVRDFQHLRPPLVADGIVGPATAGAMGLRF

SEQ ID NO: 42 - protein, 311 aa, synthetic construct
MPILRANVDYAFQIARARNRKPYGYGGVWVKNDVNRTTDCSGIVTHVLDALVNGERMTWSRHGLSTEAYRYVGGPGSRGPFGTIRVARPGDIPADAALRIGLMHGPGGGANSHMACTLEGVAIESRGGTVASGGGQWVGGSGRHYNNPLFHDWFYLPGPIVGTGTTTPTTPTAPGAVYLGRDCSRYECTGERVRALQARLNRDYAAYSDLEEDGEFGPLTEAVVKQFQLRSALTADGIAGPATLAALGLSFQPQEVPPVTAPQPVIVGPADDQLTMRWNALGGKTLVEAVAEIRDAVLGTEDRGKPGVVTK

SEQ ID NO: 43 - protein, 546 aa, synthetic construct
MTEKVLPYDRAVVPQEYGWSCGPAATQVVLNSRGIVVSETSLLNQIEAIENPGRGDDRDGTDYVGLIERVLDNIVPDARYTSVYLEKDPPTAAQKDLLWTHLKRSIDAGYGVVMNWVAPPSNKPRGVKGSVSPRYSGGTTYHYVAAMGYDDNPALRAVWIADSGFQPQGYWISFDQCASLIPPKGYAYADVTVTPPPAGSNADLVAILAQAMSPSSMPRETFENYLPFFAEAMRAAEINTVRRAAAWCSQVGHESGGLRYMAEIQTDGPGWTEDRKRYRGRGPIQLTWSSNYRKFGIWCKEKGYITDSEIFVNQPELVEQPKWGFLASSWYWLFDGPKPGQINGYADAGDNFSVSRCINGWITNDDGSARTPNGWDDRQARYNRCLALGEQLLKLTDAAGGDTELSAEAERMIREMYQEWRKEKIGPSRSFLAPDSNPIESPLGFIYNIDGNEWNGLNVWAYLFGVPFAVEAVERVAREGVHPKSYAATVPFVAEFGQAFCKGLVEFKAKLMPLLEGFDVQALRAQVEKPKADPEPWTGMSGFDDF

SEQ ID NO: 44 - protein, 503 aa, synthetic construct
MARRLFRGRQFSENGWPYVDQGSCTWDEVVPGVWLQIQNGAPFTIMRAFARDFHAHVEPLRDYDSACWTQDNSVDTSNHPGGTGMDLNWNGADQKTFRYGITKERAYPGDKARKLDELLAFYEDVIYCGGYWSIRDWMHFQMGYGTYDSKADRPTQKTLDFIARKIRPDGFSTFNRGGGGSSTESAAAVLARATGVTLTKAQSILPAVRDGLIQSECTTVPRIAMWLAQCGHESAHFEATEEYQNGPMDQERWIYKGRTWIQLTWRSAYEGFGKWCHARGLVTDPMVFVNNPRSLADLKWAGLGAAYYWTTTVRSTRKYPTLNQASDARDVLVATQIINGGTNGLTDRQNRYNRAIAVGDALLQIVQEEDGFLSALNPAEQRELLDLMRWVAAPEYGELRKLFANEDMYREDNVRRRTFAGVAIDARTFGWEDRVEKSAERGELWAIDLVVRAARGTLPGVIRPGTNTPDQFLVNHARQVLADVERINPEALKKYLALEGGSR

SEQ ID NO: 45 - protein, 526 aa, synthetic construct
MVTRANVEATKAFIRARLGNPYVYGGALSENVRQGTDCSEVWQTVLEMVHGRWKQGRQQEGATTESYRYIDVGQVGPFGTIRVAHWRDIPADAAARIAFHHGPGGGANSHMWGELDGMRIESGGSKGLVTGDRAMAVESNYATAWAYLPGPIGGTGSLGIGSTGPAVLALQHKLNERGAGLDVDGDFGPLTASAVTRAQQELHVVGDPPGVAGEATLRALGLLGADGAIPAQPNVLAAEYLAEATGVSVQKAQQMLPTMRDGLKQADATNVNRIAMFYAQTGHESANFNATEEYASGAAYEGRHDLGNDYPGDGVRFKGRTWIQITGRHNYGEFSKWAHWKGLVPSPTYFLDHPQELSDLKWAGIGAAWYWTVQRPDINALSDRRDLDTVTRRINGGLNGIDERRTRYNRALAMGDKLLTVLASNEPQDEWDALMADQTRYPSRAWYRSDDVPAHTPLDLLRNIDGMKWDERVTQAAFRGEVWAIEQIAKVARGEGPEKSQAAIEESKNLVRQLKAALAAQIGDSK

SEQ ID NO: 46 - protein, 520 aa, synthetic construct
MTERVLPFDRKIIRQDTGYWCGPASTQMALSARGKYVDEATLARECKTTVNGTDNVGLIERVLDVRLPEGNYTSMYPGGVKIGSPARPAAERKTRFWWDIVRSIDNGFAVILNWVVPPARKPIRAVKGSTNPSYGGGTTYHYVTAVGWSDEGNNGRPAVLIADSGFAPNVYWVDLDTAFALIHTDLWKGYAYADLPLIAPPPPGVEVPPGIPVKPGTPPVAVTPPVPAPPTQPPPTKLGSLTDPFTGALWSPNHYDGRGGLGTPGWIAVHTQEGGRTARDLALFLANPANEVSYHSVNDDIEVLKCVAETDAPWSASNANKYAFHHCFAGSYAGWSRDKWLSPDASDGKNEDVQLTKGAHVVAWWCDKYGIPAEWIGGRAQPPWGARGILGHVDLGQWGGGHFDPGGNFPVNEFIRRVVTFLTGEVQPPLVPLPPVVQPGTNPDAYSDWMLVRGDPRNDVDRVMRVQSRLKRAYAAYAGHLDVDGDFGPATQAAVREFQRRSNLVADGIVGPMTAAALRP

SEQ ID NO: 47 - protein, 454 aa, synthetic construct
MDINIMRQALSPTSASDTWLAECLPHMEEAMRAAQINNPRRASAWYSQIGHESAGLRYMAEIKTSDPSWSWDRTRYRGRGPIQLTWQGNYRKFGQWCKDQGYINDPELFVNQPELVEQPKWGFLAASWYWLCAGPRPGQINGFADVGDIVAVSRCINGWIETKLPNGMPDRQQRWDHCLALGDAIVPGNITPPKEIPVTWTGDPVWLEEVLRPALGDRLKTLPGWQNSGHGDFKDIRGVMVHHTGNSRESAQSIRNGRPDLAGPLANIHIAPDGTVTIVAVGVCWHAGQGSYPWLPTNNANAHMIGIECAWPTPRPDLPKGYDPAERWPDAQIISMRDTCAALALKLELPATRVIGHKEWAGAAQGKWDPGNLDMSWFRGEVRKDMEGFVFPGEHPPIEPQPGPTLPPDYAKETWDQLRIQWPQLGGRTLVDAVAVIGEKLGIEGFYDVKKTQS

SEQ ID NO: 48 - protein, 451 aa, synthetic construct
MGSENGWEPARLAPDSPLLVWKIVPGTNPPVHLQVMRGFPEVFLIAWAADWNEFIEPLRDADSACYTPTNSVSTSNHLNATAEDLNWNSHPFRKRGSLNAQQMAVLKEMEDFYEGWMFWAGRWQNPIDEMHSQCGYGTWNDNDRGFDFIHRKIRSDGRSTFRRGSLPGENLPAAAPAFPPIAPAHDDEVAATVLYDAVPIIDMDRARKLLPLVRAGLVAAKCDTPRKIAMYLAQVGWESDGFNATEEYAKNGRYAPFIGRTWIMVTWQSNYAAFGRWCYDRHLVSDPDVFVKNPRKLADDEWAGLGPAWYITDARPNINAMADAGDLLGVTKAINGGTNGLEDPRPGVPGRRTRWNQAIALGERLLELINHPDTEDTMPGLTDDEQRELLVNTRWLREQLEVSRPDWSPDADLGTDSQGRPNTLRTAVAKILRLVDKKPAAGPSSTPPATS

SEQ ID NO: 49 - protein, 438 aa, synthetic construct
MADRFFPMRDGTYTLSSGFGARWGTQHRGLDFAAKDGTPIYAAQAGTVAYIGPAQGFGQWIVIDHPAADGAGTTVYGHMWNAFATGLKAGDRVQAGQLIAYVGANGQSTGPHLHFEVHPTVWRQGSQIDPLPWLHGSRNPGDAPAPAPADPPASAPLGGQRMQDPFTGEVWSPNRSVRQKPAPRWIAIHTQEGGRTARDLCEGWLAKRESQVSYHVAVDDREILKVVAESDRPWAAANANDYAFHVVAAGSYAGWSRGKWLETDASDGKNEDVELTNLAKVCAWWCQKYGIPAEWIGGRGVPWGLDGICGHEDFGAWGGGHHDPGPGFPADELIRRVRALLGGSTPEPLPPAPPVALPGTNPDQYAGVLLYRGRPGQDPRQVRVLQTRLKRAYSKLDVDGIFGPHTEACVRDYQHLHPPLVADGIVGPATAAALGLVF

SEQ ID NO: 50 - protein, 316 aa, synthetic construct
MAFVSRYGNWYSENGWRMCDANELDRGAVPGTTLVLPIRKGIANLILKAWVAWFHRNVESLNNARGFSDEGAWTPTNDVANSNHLSGTAVDLNWSDHAFRVSYSGFTQAEINKVREGLRLFEGTIWWGQDWTSPKDPMHFQLNYGEGDARNAAFATKLQNGYLGIWSGGGGVIEPPAGVDDIYAQEGDSGDRVKRLQQFFNDNFKSYSQLEVDGDFGPATKAVVVEFQKRVGVLADGIVGPITLAAMVKHGFKTESGVKVANLPEDWTDRQVLNDIWEQLRGPDGKGWEQLGKNAKGENLSLVDAVAEVKKRLEAA

SEQ ID NO: 51 - protein, 458 aa, synthetic construct
MANRVVYGNSFSSNGWPMVDQGSCTWVKIPGTSVTLQIQNGQPLAILRAFAADFHAYVEPLRDADSACWTPTNSVSTSNHLSGTAMDLNWNSHPFQVPDAGFDAAKKARVKELLDFYEGTVFWGNDWTSPKDAMHFQLASLRNGGNINTYGNPFVDDFISRKIRPDGFSTFRRGDSPPADVAKVLADATGLTPARAREILPMVTDGLRAAECTNVNRIAMWLAQIGHESAGFNATEEYDHGRNHGDPNEVTDRWKYKGRTWIQITWQSNYAGFSKWCHGRGLVPSPSYFVDRPRELAELKWAGLGPAWYWTVARADINALSDKRDLDTVTRRINGGANGIADRRNRYNRALALGDQLLSLISEGGDDLTPEQDRMLREVHACLFNPISSQSKYKAEGEGARWRLHELIKNDDALLHETVVERQAMMGNPEALALVKREADKGDKWAQVVYRYCTEEDA

SEQ ID NO: 52 - protein, 402 aa, synthetic construct
MTTKDQVAQITIAEAKARGYTRSECLAIMSTFYQESGWNDTIWDPTHTTYGIAQQDGSYPHRFDGAAAQIKGFFDKLDVWRAKPGASTDIWLNICWMQQAPNWPSADYWYANGRRAYLTEIKSRIATVTPYLDKYWPTTGGTDVPETRPPYNEFPIWSENHYNTKRTVNDIDAFLLHTSEGFVGRDDAAEALSLWYQPRSRQVAYHYAVSQASDGGVTVVDNVDTDYASWSALSANGRSINLCFAGTRAAWSRNEWLGKFGNAIDVAAYLAVQDCKKYNIPTKVIAPPYTGRLPGITDHRYVTQILKDGTHTDVGDGFPWDYFTERVNHWAAGGKTDPEPPKVKRFPDDWTDRELAVETLRQQRGYTLNGWPQLGGRTVVDVLGAIGAKLGVEGCYDVKDKS

SEQ ID NO: 53 - protein, 311 aa, synthetic construct
MPILRANVDYAFAIARARDKKPYGYGGVWSKTDVNRTTDCSGIVTHILDALVNGEKMAWSRHGLSTEAYRYVGPAGSRGPFGTIRVARPQDIPADAALRIGLQHGPGGGANSHMACTLEGVAIESSGSYGQRVGGPARGYNHSMFHDWFYLPGPIVGSGTSTPATPTAPGAVYLGRDCSRYECTGERVKALQARLNRDYPAYSDLDEDGEFGPLTEQVVREFQSRSNLTIDGIAGPATLAALGLSFQQQPAAPAPAPAPAAPKPVVVGPADDQLTMRFNCLGGQTLVEAVAEIRDKVLGTDDRGKPGVVMK

SEQ ID NO: 54 - protein, 321 aa, synthetic construct
MAFIQKQGYWKSENGWRMCDTAELDYTPVPGTNFKLGVRKGAPSVILKSLIWRLNRIEPMITSQIGCYTAENSMRNSNHNSATAIDYNWNLHPYQKWGTWGNNRAAVDKVIADFRGIVEFGGNWTSPRDEMHFELHFAEGHQGTEQLAQELRDGLWGIWKAGTAPSDAPSSGGGSSAGDILRIGSTGPEVLKMQKGMNAVFKNYKAMPLDEDGIFGPMVQAAVIEFQQRSLISVDGEVGPQTKAKLAEYGIVLTGQTTPTSPPAPAAFVYPSHEEMVKQLWEQAFGPQAKGWESLFGKSVDGSRGKFTVEAIADIRNVATQ

SEQ ID NO: 55 - protein, 352 aa, synthetic construct
MPWKGDPVWLPDVLRDWRVPFSISEGAFQRGHGDMGDIWGVVDHHTGSDNASWQSIAFHPSLGLASQLHLGFNGHVTICGVGIAWHAGNGSWPGLGTNNANPRTIGIEAANDGGGKPGRPHRADWPVAQYDNYVKMNAAILSFLKYGSDRSIGHKEWAGAAQGKWDPGGIDMNIFRRDIQSLLTIPAKPLVNAIDEHAKVAGAWLGKRVTSGEQPTADGKGRFAQFENGYIYWSPDTGAWAIPRHLFETYAELGYEAGPLGYPIAPHDVVEGGDVQAFQRGVLYRKYGESGHFITGAIGNRYRLNQYERGFLGWPTTNETKQGGMIWQGFEHGRIAFSPDGTIVLNERNDFV

SEQ ID NO: 56 - protein, 403 aa, synthetic construct
MALGAPMENGWPECDLSDCDYATVPGTPLRLPFRKGHPFIILQAFLRDLNEFVEPLMNARGATDEGSWTDNNSVYTSNHKGATAFDYNWSDHPMGNALAGWHGSVLISGEQEPAVRELLRYYTYRGIQLVWWGNDWYSPKDSMHFQMGYDTVNNPGIVQEFIDKFIRPDGYSKYRRDGSSGPVELTVPLIPAANGRWTSPSPAWAHLITRESGGNPTIIQQIHDVNSGGNEAEGLFQITPRTWKAHNGTEFATSARFATPQQQAIVAARIITRNPSGSDWGAGLPGREDAAQLRAGLVPTQTTNDPLEELMAMEVESFSIYANPGEPKIPVHVMIQSLDAHGPHEPYIEEQARHGDRDAIFRVARTAAGKGKYGDAPGPVKQASRVLKELEEAGVLAEYLKGN

SEQ ID NO: 57 - protein, 453 aa, synthetic construct
MGSENGWEPARLAPDSPLLVWKIVPGTNPPVHLQVMRGFPEVFLIAWAADWNEFIEPLRDADSACYTPTNSVSTSNHLNATAEDLNWKNHPFKQRGSLNAAQMAVLKEMEDFYEGWMFWAGRWQNPIDEMHSQCGYDTWNDNDRGFDFIHRKIRSDGRSTFRRGSLPGENLPAATPAFPPIAPAHDDEVAATVLYDAVPIIDMNRARKLLPLVRAGLVAARCDTPRKIAMYLAQVGWESDGFNATEEYAKNGRYAPFIGRTWIMVTWQSNYAAFGRWCYDRHLVSDPDVFVKNPRKLADDEWAGLGPAWYITDARPNINAMADAGDLLGVTRAINGGTNGLEDPRPGVPGRRTRWNQAIALGERLLELINHPDTEDVMPGLTDDEQRELLVNTRWLREQLEVSRPDWSADADLGTDSQGRPNTLRTAVAKILRLVDKGKPATSVANANPPATS

SEQ ID NO: 58 - protein, 305 aa, synthetic construct
MSFRTAYGNAYSENGWRMCNRDECVTVSGPYMNTAPLRRGPAEKLLGEFVRRYHQVCAPVVSPVWGWSETNDVGNSNHLSGTAVDVNAPQWPWGLRKMPADLVARINVLLDQFEGAIYWGRNWNRPDEMHFQLNWREGDAKYDRIIAKFSGGSVPVTPHVPNLPTDDSELLMRGSANVEQTRILQAGLKKVFPSYAGHLEVDGDYGPETEKAVRRFQSGSGLVADGIVGPATRAELAKYNIILKPAAPQEIIPVGKTPVVVGPADDQLNMRFNCLGGKTLVEAVAEIRDAVCGTNDKDKTGVVLK

SEQ ID NO: 59 - protein, 502 aa, synthetic construct
MARRLFRGRQFSENGWPYVDQGSCTWDEVVPGVWLQIQNGAPFTIMRAFARDFDAHVEKLRDADSACWTQDNSVDTSNHPGGTSADFNWNGADGRTFRYGITKERAYPGDKARKLDELLAFYEGVIYCGGEWSIRDWMHFQMGYGTYDSKADRPTEKTNDFIRRKIRPDGWSTFTRGGGGAPAPAADAASIVARATGVTLAKAQEILPTLREGMVLAECTTVPRIAMFLAQLGHESAHFNATEEYQNGPMDQERWIYKGRTWIQLTWRSAYEGFGRWCFARGLVSDPMVFVNNPRSLADLKWAGLGAAYYWTTTVRSTRKYPTLNQASDARDVLVATQIINGGTNGLEDRQNRYNRAIALGDELLQILGEEDSFLSALNPAEQRELLDLMRWVAAPEYGELRKLFANEDMYREDNVRRRTFAGVAIDARTFSWEDRVEKSAERGELWAIDLVVRAARGTLPGVIRPGSNTPDPFLVNHARQVLADIEAINPEALKNYLKGAS

SEQ ID NO: 60 - protein, 493 aa, synthetic construct
MARRLFRGRAFSENGWPYVDQGSCTWVRIPGAEHVSLQIQNGPPLQVLRAFAADFHAHVEPLRDPDSACWTQDNTVDTSNHPGGTAMDLNWQGADGKTFRYGISEERAYPSPKHQRLRELLDFYEGVVFCGGFWSIQDWMHFQMGAGTYDSRADRPTEKTLDFIRRKIRPDGFSTFKRGGGGSAAPDAASVLARAADIPLAKAQEILPTFREGAVLAECTTVPRLAMFIAQTCWESDRYRATEEYANGPMNEERWIYKGRTWIQLTWRSAYEGFGRWCHARGLVNDPMVFVNNPRSLADLRWAGLGAAYYWVTTRRESRKYPTLNEASDARDVLVATQIVNGGTTHLAERTAIYNRAIALGDELLHILGEEDELANPEIEKMIREVHACLFNRIVSQSIYRFPKNPDGSEHPGNIWQLHELIKNQDGMAHLKYVEDSARGGDLTELDRIAVVAAGRGAVRDQWAVQRAQRVLADIERTNPEVIRRYLAQKGAA

SEQ ID NO: 61 - protein, 523 aa, synthetic construct
MTEKVLPYDRSIVPQETGYWCGPASTQVILNSRGIIWKESDLAREIGTTTRGTDYVGLIERVLDRVTPDANYTSVYTERDPMTNDQKETFWRNIVQSIDGGYGVSTNIVAPPNNKPRGVLGSVSPSYSGGTTYHYIAIMGYHIGDDGFRAVWVADSGFRPFGYWCSFDQMATLVPPKGYCYANTGPVVPPTAPQIVPVALFSKAMGDTIGLDQYAALLPGFVDACEKLGVTENNEIAMLAAQFGHESVGLKYMRELWGPTADQLNYDHMMGNGPGEGRKYLGRGPIQVTGKDNYRELSRWAFDQKYIDSPTLFLDQPELLEQPQFGFLGAVWYIKVKQPRFMEYARAGDIENASKAINAPAWIGTQNRARGIDDRIARWNRCRAMDLMPLLREEDDPLSDPILAKLIGEIHGALFNPIPSLSRYREDYEGNALTTKDIPRNVDKFSHEEWTETAAIRGNPKAVKMVARNASRGDSMAMDVFARIPAETLAANGIDSAAVKSGYDKSQAEDPNRSVFALPPKGN

SEQ ID NO: 62 - protein, 1366 aa, synthetic construct
MALYRTLYPLTYRSGNTVMHVTKPGVIIDLDRNQAQELSGRVVYVGDLITYPRSGVMYYDSVDSFPLEGDERYIFLARQEGELYGWSYSDGYKLFGAAGQVDEEALAEAVGDVIEDALGDALAGKANVSHTHNPSDVTGLPAAIAKLAGIDAGANVTNATNVNAAGAVMNSDTSTADMQFVVDEDNMASNSATKVPTQQSVKAYVDSRVGGGGAAGVNYGISTLRIDTNDVPIADRETYIEATFEIEGITYNGEIRGRGNSTWDFPKKPWRVRLDSAAALLGMPSSRHWALLANYIDRSAARNAIAMQIGSRLSGLDWTPKYRYVEVVLNGTYEGLYQLMEIVRFDPNRVDAEAADDTTGLGLTGAYLLEIDSYRDADVVIDTVHDDLPIIMDDPDGSVTEQATYIEDWLNNFETVLYDDDEWLDPETGYKTLIDLDSFVDWYLVNELLVSIDAGFETSVKMYKTRDTIDTPGKLFLGPLWDYDQSMGRAGSTTFSHEGWWLLETTQPASGTPYPGATWIVRMMSDPDFLDAIEVRWPQIVALLDDLEDLVTKTLRHIALGRVNDQVLWPDGGTWSTNASEIVDWLEDRMEWITDNLPALGVSDGEPPSIPEGLDYTATGSTINVTWEASTDNVGVTGYEVRIDEGTPVAKTGLSHTFTDLEPETEYSIDVRAKDAAGNWSDWSEPLVAETGEEGSLPSPISHFDWHEGSGSTTTSIVSGHVLTAAVPSSAWSGTSAAGQFEGDIGDTATPNWTIAVDFTLNAGSSWRNILHTSGAGAADEVWLQLNGSTLSIWTSAGASTGFDPAQPTLPPNLPIQLVMTNDGTTQRVYVDGELVSSRARNNILEPTSAVIMDTSEKLNGYVHSLRFWDVVLSDAEVEALMPDSGDEEDPPLFRGMTDEITQYGITWTFDQEYMAGQFITGDWWVVGPVTVTSVSPAPQGRLDGEDDDDLTGEAGQRVDDRMRHGSMVLTDVTTDQAYDSRCRFYNPARAISYPYNLAANRSLISSVSAVHPGGSEMGDLADGAEGRVMETAAVLTCLDEEPPEDAFRPCYWGADKSKIVRASDIQWDRLANVTPASSPPTLVEYARFFERPWLDHMQAYWVGQFLWPWKNQPGYGRGYAAVVGTAGLLVNCNYTEAQKRPIVHGLIQLGIDLAGMLKADSCFRVSGGHGSGRKFPVVFAHLMLDDDPYFQFEADAVGADVGEDGDGEPQALFGEDASTYWGETYYGAPACFQMVGYSSEVPPHQESPPPYTDWDDTSEGYRQCCTVGAWVGQTLAILLMGAKAAWNHDSYFQVVDDWMADPDPYAANRGGHPRPDDWQETNAWHPWATRMWNLHRSSVPAQDDGTDNYKWMALDREWVPNPPPA

SEQ ID NO: 63 - protein, 492 aa, synthetic construct
MALGMTLENGWPECDLSDTERLTIPGTALSLPIRKGQPHAILQAFFRDVNEFIEPVMNARGLSDEGSWTENNSVYTSNHKGATAVDWNWSDHPLGVKNGGWDGSVLINGSQVPAMRELLTWYEGMVFWGNDWSSPVDSMHFQMGYNTCCGGDNAKRVDSFIQRKIRADGFSTFRRGGTPRGGGFAELPAAPVHPIKPTSGLTPEVLWRIAGGAASKLPVSHFERWFDELVECQAACGVLGNIDRSAMWYAQVFHESGNLVHTEEIASGAAYEGRCEGLGNCQPGDGVRFKGRSFIQVTGRSNYTKLSGWAHSKGYVPTPDYFVVHPDQLDDEQYAMLGVTWYWTTQRRMNDAADARNLELATRYVNGGTNGLDHRRAIYNRALAENANLLLTNPVEPWEELMATAVPSLSIYANPGEADVPLAVMIAALDAHGPHEPYVERQAQEFGDVDSIRRIARTANGQGRVKTPAAIKQATDAFRLIPPEFVRAAIPA

SEQ ID NO: 64 - protein, 472 aa, synthetic construct
MAVTRANVEATKNFIRARVGNPYVYGGALSPTNVRQGTDCSEVWQTVLEMVHGRYNPGRQSEGATTESYRYIPVGGVGPFGTIRVASWRDIPADAVAKIAFHHGPGGGASSHMWGDLDGMLIESAGSKGLVTNGRAMTIDNSYATAWAYLPGPIVEDGSPIPEDPNAVTWGIDISNHQGEMDLNRVKAEGFDFIWCKVSEGANYRDPFWPGNRDKARAAGLILAGYHYVRTGDPAAQAKTFVEHLGDKSIPAMLDFEDGSGNIEQFWAVKAEIEKLGVQVRLSYIPDWYWERIGKPDLSKVPGLISSEYVSGTGYASVLYPGNSSNFWKAYGGRTPDVLQFTDRALVAGKSVDANAFRGTPDKLRRLLGAGGDDFLSALSDAEQRLLFDRTNQLWGALFNPIASLSKYRAEGEGEVHKTKDLVRNIDAMTHETLVERQAMMGNPEALALVKREADKGDKWAACVYKYCTEEA

SEQ ID NO: 65 - protein, 393 aa, synthetic construct
MSAKDDYARAVIAEGKRRGITPRGIQIGLATVDVETGFVMYANSKVPESLTIPHDRVGSDGFSVGLFQQQIVRGANGQWWWGDCATCMNPTLSAGLFFDRLAKLPYNDPGRSPGSFAQQIQQSAFPDRYDQHFAAAVDLYNRLEGDVVVDRPDFNEYAVWSPNSQSRNGAKIDLFLLHTEEGNSNADQLANGILSDPAPGGNPANAVSYHYTISMDPNDKGVTVCDVVDTDEASWSVGNANNRSINLCFAGSKAAWTRQQWLDNAGKAIDIAAYLAVQDCKKYGISINVAPPPYNTGTPGISDHNYVTTVLKWGSHTDVGPNFPWDVFTAAVNKYADTGTVATPINPPAFTYPSTDVMIREIWEQLRGPQAKGWSQLGGKSLVDAVASLTGGK

SEQ ID NO: 66 - protein, 459 aa, synthetic construct
MSFIRSAFADKPLLTREQWMKLFIRVADELDMPDRRGAAVLAAMCAFQEAGADPHNTGKRQIWVPGNNADPCFKANPAAYPHDSMGDDGQSTGPFQQQMNKPGTTPWGWGGNYGDPEGTRKRMDPYESTKMFMGHPSSGLKKKGYDASNAQTANDAIQRVQGSGVPWAYAQWWDEANLLYDEVVGDVPPSTGGGKVPVSGDPVWLEEVLRKRLGDRLVVHDGWKEYGTGGVMGEIWGVMIHHTGNRNASWESIRNGRPDLRGPLSQALIAPDGKFHLVAVGPCNHAGVGSYPGLGSDGNRRAIGFECAWPTIRPDGSFDKGERWPDAQILTMRDATRAVLEHLGHDHTHVCGHKEFNKVDGKWDPGNMDMNWFRGEVRKALAGEFDPKAPEPEPQLPSQPGIPTDHDLLVYDQICGRWEMLGWRTPVEALATILDELRGTKNAGSRGFTRGPHDIEDKK

SEQ ID NO: 67 - protein, 458 aa, synthetic construct
MANRVVYGNSFSSNGWPMVDQGSCTWVKIPGTSVTLQIQNGQPLAILRAFAADFHAYVEPLRDADSACWTPTNSVSTSNHLSGTAMDLNWNSHPFQVPDAGFDAAKKARVRELLDFYEGTVFWGNDWTSPKDAMHFQLASLRNGGNINTYGNPFVDDFISRKIRPDGFSTFRRGDAPTVDVAKVLADATGLTPARAREILPMVTDGLRAAECINVNRIAMWLAQIGHESAGFNATEEYDHGRNHGDPNEVTDRWKYKGRTWIQITWQSNYAGFSKWCHGRGLVPSPSYFVDRPRELAELKWAGLGPAWYWTVARADINALSDKRDLDTVTRRINGGTNGIADRRNRYNRALALGDQLLSLISEGGDDLTPEQDRMLREVHACLFNPISSQSKYKAEGEGARWRLHELVKNDDAMIHEMLVERQAMMGNPEALALVKREADKGDKWAQVVFRYCTEEDA

SEQ ID NO: 68 - protein, 327 aa, synthetic construct
MAFIQKQGYWKSENGWRMCDTAELDYTAVPGTSFKLGVRKGSPNIILKALIWRLDKIEPMITSQIGCYTAENSMANSNHNSATAIDYNWNKHPYQKWGTWPNRAAVDKIVDDFRGIIEFGGDWTSPRDEMHFELHFAEGHAGTEALAKDLANGLWGIWKPGAAPAPTPAPGGGNDGILRIGSEGPEVLKMQRGMNSVFKNYRAMPLLEDGIFGAKTKEAVVEFQQRSLIDVDGEVGPQTKAKLAEYGIVLTGATAPTAPPAIVPQKVWPQTASDRELLEYIAAQLGPGDPSWPIKLGNNEKGDPLTVRDRLGLTAKDIEEIKGRLSQ

SEQ ID NO: 69 - protein, 423 aa, synthetic construct
MSFTWFADKPLRTREQVAREVHAVSLARGLDELATVIALMTISTEVGTGTGDDRKWWCPANDRVPATKNYPHDSRSDDNRSSGYFQQQPGPNGEPWWGTPENMMTLPQAANTFLERLSDDYRRAANNPRLAGEFAQRVQQSAYPDRYADKWDEAWSVLRRALNETTPEEPVTENRPAYNEFPIWSANNSARSGKPTMFLIHTQEGGGGDAAAENLAKWFQNGNGVSYHYTISQASDGGVTVVDCVDTDRAAWSVGNANSISINLCFAGSRASWMRDQWMKQSNAIDVAAYLAVQDAKKYGFTPLVVPPPYTNGRPGISDHRWVTDVFKWGTHTDVGDWFPWDYFTERVNHWAAGGKTEPEPPKVKRFPDDWTDRELAVETLRQQRGYTLNGWPQLGGRTVVDVLGAIGEKLGVEGCYDVKDKS

SEQ ID NO: 70 - protein, 402 aa, synthetic construct
MPVYAIQSGTVIYAGAASGYGGPDPAGWLVIDSDDAEGGGCLEYGHIVREVNRGDHVTAGQRIGHINPNNRTNGGVAPHLHVTDWPHAYGQGSRQNVMARLSGAREPEAQAAPTPTEQGTVVGDPVWLADVVRPTVAKFAEYPGWRNRGHGDFKDIRGVMVHHTGGPASAASIANGRPDLAGPLAQLHISRDGTVTVVAVGVAWHAGAGSYPWLPTNMGNWHLIGIECEWPYGEPGINERNAYTVRWRDPEIIALRNTCAAILLRLGLGVDRLIGHKDYAGRAQGKWDPGNMSMDWLRGEVRKDMDGFVFPGEDIVNVTPPPVPAPVPAPPATANVLLHRGMSGPNVVKLQTTLRRWYSKLAVDGDFGPHTEACVRDRQRIYGLDVDGIVGPLTAAKIGLVL

SEQ ID NO: 71 - protein, 473 aa, synthetic construct
MAVTRANVEATKNFIRARVGNPYVYGGALSPTDVRRGTDCSEVWQTVLEMVHGRYNPGRQSEGATTESYRYIPVGGVGPFGTIRVNHWRDIPANAAAKLAFHHGPGGGANSHMWGELDGMLIESAGSKGLVTNGRAMTIDNSYATAWAYLPGPIVEDGSPIPEDPNAVTWGIDISNHQGDMDLNRVKAEGFDFIWCKVSEGANYRDPFWPGNRDKARKAGLILAGYHYVRTGDPAAQARTFVEHLGDKSIPAMLDFEDGSGNIDQFWAVKNEIEKLGVQVRLSYIPDWYWERIGKPDLSKVPGLISSEYVSGTGYASVLYPGNNSNFWKAYGGRTPDVLQFTDRALVAGKSVDANAFRGTPDKLRRLLGAGGDDFLSALSDAEQRLLFDRTNQLWGALFNPIASLSKYRAEGEGEVHKTKDLVRNIDAMTHETLVERQAMMGNPEALALVKREADKGDKWAQCVLDYIEGDES

SEQ ID NO: 72 - protein, 451 aa, synthetic construct
MGSENGWEPARLAPDSPLLVWKIVPGTNPPVHLQVMRGFPEVFLIAWAADWNEFIEPLRDADSACYTPTNSVSTSNHLNATAEDLNWQSHPFRKRGSLNAQQMAVLKEMEDFYEGWMFWAGRWQNPIDEMHSQCGYDTWNDNDRGFDFIHRKIRSDGRSTFRRGSLPGENLPAAAPAFPPIAPAHDDEVAATVLYDAVPIIDMDRARKLLPLVRAGLVAAKCDTPRKIAMYLAQVGWESDGFNATEEYAKNGRYAPFIGRTWIMVTWQSNYAAFGRWCYDRHLVSDPDVFVKNPRKLADDEWAGLGPAWYITDARPNINAMADAGDLLGVTKAINGGTNGLEDPRPGVPGRRTRWNQAIALGERLLELINHPDTEDTMPGLTDDEQRELLVNTRWLREQLEVSRPDWSPDADLGTDSQGRPNTLRTAVAKILRLVDKKPAAGPSSTPPATS

SEQ ID NO: 73 - protein, 491 aa, synthetic construct
MARRLFRGRPFSENGWPYVDQGSCTWDEVVPGVWLQIQNGPPFTIMRAFARDFHAHVEPLRDADSACWTPDNSVDTSNHPGGTGMDLNWNGADGRTFRLGISKERAYPGDKARKLDELLAFYEGVIFCGGEWSIRDWMHFQMGAGTYDSKADRPTEKTLDFIKRKIRPDGFSTFGRGGSTAPVADGASVLARATGIPLERARQILPALREGLILAECNTFPRIAMFLAQTCWESDQYRATEEYANGPMHEERWIYKGRTWIQLTWRSAYEGFGRWCHARGLVSDPMVFVNNPRSLADLKWAGLGAAYYWTTTVRNTRKYPTLNQASDARDVLVATQIVNGGTTHLAERTAIYNRAIALGDELLHILGEEDELANPEIEKMIREVHACLFNRIVSQSIYRFPKNPDGSEHPGNIWQLHELIKNQDGMAHLKYVEDSARGGDLTELDRIAVVAAGRGAVRDPWAVQRAQRVLADIERTNPEVIRRYLAQKGAA

SEQ ID NO: 74 - protein, 288 aa, synthetic construct
MCNRDECVTVSGPYMNTAPLRRGPAEKLLGEFVRRYHQVCAPVVSPVWGWSETNDVGNSNHLSGTAVDVNAPQWPWGLRKMPADLVARINVLLDQFEGAIYWGRNWNRPDEMHFQLNWREGDAKYDRIISKFSGGSVPVTPHIPNLPADDSELLMRGSANVEQTRILQAGLKKVFPSYAGHLEVDGDYGPETEKAVRRFQSGSGLVADGIVGPATRAELAKYNIILKPAAPQEIIPVGKTPVVVGPADDQLNMRFNCLGGKTLVEAVAEIRDAVCGTNDKDKTGVVLK

SEQ ID NO: 75 - protein, 516 aa, synthetic construct
MTEKVLPFDRKIIRQDTGYWCGPASAQMALSSRGKFVDEATLARECRTTTNGTDNVGLIERVLDVRIPDAKYLSVYPGGLKIGTPARPANERRDYFWWDVVRSIDAGYPVILNWVVPPNRKPIRGVKGSPNPSYGGGTTYHYVTCVGWSDEGNNGRPSLLIADSGFAPNVYWVDFETAFQLIHSDNYKGYAFADHPLQPPPAGVKVPPGVPIAGTAPTPAPAPAPPPAIVKPAGKIADPGVYLLTKANRYENRGGKPWPFWIALHTSESRSRARDLRQYCETHEVSYHGIGDDREVIRMVRDEDGSWSAVGANNLAYHYCFSSSFAGWSREAWLDPNPADGYNEREALRLGAKQVAFWIQLSIERGRPIPLEWIKGRGTPPWGHNGICDHSSFGAWGGGHSDVGHNFPVNTFMDDVRFWLTGTEAPPIAPAPPVVVAGTNPDRYADWLEYAGKPNPNRDRVAAIQAAMNRPPTPFGLDVDGIFGPLTRLAVIGFQQHVHLVADGIVGPMTAAALNP

SEQ ID NO: 76 - protein, 329 aa, synthetic construct
MAFIQKQGYWKSENGWRMCDTAELDYTAVPGTSFKLGVRKGSPNIILKALIWRLDKIEPMITSQIGCYTAENSMANSNHNSATAIDYNWNKHPYQKWGTWPNRAAVDKIVDDFRGIIEFGGDWTSPRDEMHFELHFAEGHAGTEALAKDLANGLWGIWKPGAAPDPAPAPAPGGGNDGILRIGSEGPEVLKMQRGMNSVFKNYRAMPLLEDGIFGAKTKEAVVEFQQRSLIDVDGEVGPQTKAKLAEYGIVLTGATAPTAPPAIVPQKVWPQTASDRELLEYIAAQLGPGDPSWPIKLGNNEKGDPLTVRDRLGLTAKDIEEIKGRLSQ

SEQ ID NO: 77 - protein, 580 aa, synthetic construct
MSFIRSKFAPRALLTVDQWIAIFAAVADELNMPDKRGAVICAAMCAFQEAGADLQDGNGRQIWIPGNMADPCYADDPDAYPHDSEGNDGQSTGPFQQQMNQPGKAPWGWGGNYGDCAGTRKRMDPWDSTRMFFGWPGSGLRDKGYDASTAQRANDSIQRVQGSGVPNAYAQWWGLANAAYDRYLGNPIPAPPSPGGSSSGAPALTPNPAWRGDPLFLPQLLRAFGVSVTTYTDADGIRWDERGHGDFGKISWVLWHHTGSVNETDNGIAHHPALGLAANMLIHPDGHVVLTGSGIAWHGGIGVYPGIPEDGINQISIGIECSYGPDRDGRYTLPWPEAQMNAMIAVGGAISWFLGDTLPPSHQIAHKEWAGADNPLGVNKQGKPDPGNLDMTWFRAQIAARAAAGPTTGGDDWMTNPDAITMLAAIYRETVTQKSPSRSFMAEDGTLIDTPLGIDWNTDGNAWTLVMTEAYWNDVPLAITVVEDIAANGVRQTSWAGSADKDDQVKWNQWLRDFGQAYCAGLVRRKAQWTALVAAVSALAAAQSAAALTAEATTPAAPKKRAPRKRAATKPTTTTTGGAE

SEQ ID NO: 78 - protein, 451 aa, synthetic construct
MPGSEIPRYWPLGAGRIVTSPFGPRSGGFHAGVDFGRNGGSAGMPVYAVQSGTVIYAGAAQGYGGPDPAGWLVIDSTDAEGSGCLEYGHIVREVSKGQHVSAGQRIGHINPDSRTNGGVAPHLHLSDMPREYNPGAKQDPMKRLAGAREPEAAPPAPAPNTGGNATVALADPITKALWTARNRYSPRGLPSPMWIGCHTSESRSRAVNLRDYCERNSVSYHRIVDDTDIVGMVRDTDGSWSAVGANKYAYHICWSSSFASWSREQWLDPTPGDGFNERNALRLGAKQIAYWIQQSRAAGRPIPVEWIGGRNRPPWGLNGICGHVDFGAWGGGHSDPGPNFPVDTLLSDVREFLTGEPQPPIVAPPPVGVPGTNPDKYADWMLYQGNPRNNIDRVRAVQRRLKYAYAGYAGHLAIDGDFGPLTRMAVEEFQRRSKLVVDGIVGPMTAAALKP

SEQ ID NO: 79 - protein, 525 aa, synthetic construct
MVTRANVEATKAFIRARLGNPYVYGGALSENVRQGTDCSEVWQTVLEMVHGRWVQGRQSEGATTESYRYIDVGQVGPFGTIRVAHWRDIPADAAARIAFHHGPGGGANSHMWGELDGMRIESGGSKGLQTGDRAMAVESSYATAWAYLPGPIGGVGSLGIGSTGPAVIALQHKLNERGAGLDVDGDYGNLTASAVTRAQQELHVVGDPPGVAGEATQRALGLLGADGAIPAQPNVLAAEYLAQATGLSVTKAQQILPTMQSGLKLADATNVNRIAMFYAQTGHESANFNATEEYASGAAYEGRADLGNTHAGDGVRFKGRTWIQITGRHNYGEFSKWAYAKGLVPDPNYFLDHPQELSDIKWAGIGAAWYWTVARTDINELSDRRDLDTVTRRINGGLNGIDERRTRYNRALGMGDKLLTIFANEPEDEWEALMADQTRYPSRAWYRSDDVPAHTPLDLLRNIDGMKWDERVTQAAFRGEVWAIETIAKVAKGEGPEKSPAAIEESKNLVRQLKAALAAQIGDSK

SEQ ID NO: 80 - protein, 540 aa, synthetic construct
MAVARANVEATKTFIRARLGNPYVYGGALSPTNVKQGTDCSEIWQTTLEMTLGRYVPGRQGEGATTESYRPKSMGGPIPDGGIGPFGTIVTRDRNAVPANAVAKIGLHHGPGGGANSHMWGELDGMGIESRGGGVGVITNPRSMRWDDSYANAFAYLPGPITEDGTPAAPEYVLLGRNYENSGDRVRQLQRALNSYGYGLDEDGEYGPLTEQAVGAFQRSKGLEVDGIAGPVTLAALGLTFGRPVESSPEGLALFRQIMSRTDATNAWLATCLPYYIEAMRAAEINTPLRAAAFASQIGHETSGLKYMAEIQTNGPGWTEDRRIYRGRGAIQLTWSSNYRRFGQWCREQGHVTDPELFVKNPELVEHPQWGFLAASWYWRFGGPKPGQINSYADKGDILAVSRCVNGWIEGVDPVGWADRRSRYLNCMALGARIMNLTTVDPLEELLKMKLNSMSIYRTPGEGPIDAAIMLAALDAHGPHEDYVEKMARRGDEDALFRVARVAMGKGAETTPAAIKQATDVLLEIAETNRAAVNAALGKV

SEQ ID NO: 81 - protein, 509 aa, synthetic construct
MLVALGMRLENGWPECDLSDTERLTIPGTPLSLPIRKGQPHAILQAFFRDVNEFIEPANNARGYTDEGSWTENNSVYTSNHKGATAVDWNWSDHPVEVKDGGWNGSVLINGSQVPEMRRLLAFYEGMVYWGNDWTSFIDSMHFQMGYDTCCGGDAAKRVDSFIQRKIRADGFSTYRRGGVPRGGGTAQAPAAPANPIPPTSGLTGEVLWRIAGRPDRVTVARYAALLPRLIECLHACECNTIDRRAMWFAQVFHESGGLFYTEEIASGDAYDTRTDLGNTPQVDGDGRLYKGRSFIQVTGKNNYAAMSGWAFEHGYVPTRDFFVVHPDRLDDDEYAFLGVTWYWTTQRPMNTYGDARNIEMGSRAVNGTNPKTGRANGIEDRIAIYNRALAENANLLDPANVAPLDPWEELMTLKVPSLSIYADPDEPDVPIPVMIAAQDAHGPHEPYVEKQAKLYGDADSIFRIVRTAAGGGRVKTPAAIKQANDVLDEIKANHPEFIVAALREAQKK

SEQ ID NO: 82 - protein, 305 aa, synthetic construct
MSFRTAYGNAYSENGWRMCNRDECVTVSGPYMNTAPLRRGPAEKLLGEFVRRYHQVCAPVVSPVWGWSETNDVGNSNHLSGTAVDVNAPQWPWGLRKMPADLVARINVLLDQFEGAIYWGRNWNRPDEMHFQLNWREGDAKYDRIIAKFSGGSVPVTPHVPNLPTDDSELLMRGSANVEQTRILQAGLKKVFPSYAGHLEVDGDYGPETEKAVRRFQSGSGLVADGIVGPATRAELAKYNIILKPAAPQEIIPVSKTPVVVGPADDQLNMRFNCLGGKTLVEAVAEIRDAVCGTNDKDKTGVVLK

SEQ ID NO: 83 - protein, 544 aa, synthetic construct
MDVTRANVEGAKAFIRARLGAPYQYGGALSSTNVRQGTDCSEVWQTVLESVFGRYVPGRQFEGATTESYRSVKVGEVGPFGTIRVARPQDIPADAVVKLAFHHEGNGGASSHMWGELDGMLIESASKKGLCTAPAAWPIEHSYANAWAYLPGRIVEDGAAPTVVEPQDTLYADVSEWQVPVTDAYTDAGYRVLCIRSNDGTHRDEDWANNYAWCKRAVDDGRLAFFIVYFVWRPNWQAAVDTLKAQVGEPHPKMAVMLDVESWNGQIGGDQSAGINAAFEQIAGWLGDRRRVIGYGNTGDLNALWPRKPEGVRLVVAGYGKLPTYPGMIAHQYTDGQGYGGGLPEGAPPFGRCDMNAANGLTASAFARALGIEPSTTGEDDFMSALSADEQREMLTYLRWAFAPGTGEFRKRFPSRSPLRHLGEGVIDTAVGIDLNDDANDHVVLVKELAEIGDPGALALLHEVAGADPVLYPDRQEDRKLAQRILDHLPQSSTKPAEPDTTPAPVRNVACAQSGAGCILVANGGDGSCALAGDDCVLRKGASA

SEQ ID NO: 84 - protein, 544 aa, synthetic construct
MDVTRGNVEAAKAFIRARLGNGYVFGGQLSPTNIKQGTDCSEVWQTVLELVFGRYVPGRQFEGATTESYRYVKVGEVGPFGTVRVARPQDIPADAVVKLAFHHEGNGGASSHMWGELDGMLIESASKKGLCTAPAAWPIEHGYANAWAYLPGRIVEDGTAPTVVEPQDTLYADVSEWQVPVTDAYTDAGYRVLCIRSNDGTHRDEDWANNYAWCKRAVDDGRLAFFIVYFVWRPNWQAAVDTLKAQVGEPHPKMAVMLDVESWNGQIGGDQSAGINAAFEQIAGWLGDRRRVIGYGNTGDLNALWPRKPEGVRLVVAGYGKLPTYPGMIAHQYTDGQGYGGGLPEGAPPFGRCDMNAANGLTASAFARALGIEPSTTGEDDFMSALSADEQREMLTYLRWAFAPGTGEFRKRFPSRSPLRHLGEGVIDTAVGIDLNDDANDHVVLVKELAEIGDPGALALLHEVAGADPVLYPDRQEDRKLAQRILDHLPQSSTKPVEPDVTPEPARNVSCAQGGAGCILVANGGDGACALAGDDCVLRKGVTA

SEQ ID NO: 85 - protein, 404 aa, synthetic construct
MALGAPMENGWPECDLSDCDYATVPGTPLRLPFRKGHPFVILQAFLRDLNQFVEPLMNARGATDEGSWTDNNSVYTSNHKGATAFDYNWSDHPMGNALAGWHGSVLISGEQEPAVRDLLRFYTYRGIQLVWWGNDWSSPKDSMHFQMGYSTVNNPAIVQEFIGKFIRPDGYSTYRRGSSAVVPTELTVPLTPAANGRWTSPSPAWAHLIMRESGGNPTIIQQIHDVNSGGNEAEGLFQITPRTWKAHNGTDFATSARFATPQQQAIVAARIITRNPSGSDWGAGLPGREDAAQLRAGLVPTQTTNDPLEELMAMEVESFSIYATPGEPKIPVHVMIQSLDAHGPHEPYIEEQARHGDRDAIFRVARTAAGKGKYGDAPGPVKQASRVLKELEEAGVLAEYLKGN

SEQ ID NO: 86 - protein, 498 aa, synthetic construct
MALGGRMENGWPECDLSDCDYATVPGTPLRLPFRKGHPFIILQAFLRDLNEFIEPLMNARGATDEGSWTDNNSVYTSNHKGATAFDYNWSDHPMGNANAGWNGSVLIAGDQVPAVRELLRFYTYRGIQLVWWGNDWNSPKDSMHFQMGYNTVNNPQIVQEFIDKFIRPDGFSTFRRGGAAPSAPAVDAATILARATGIAYAKAQEILPTLRDGLILADCKSVPRIAMFVAQTCWESDHYNATEEYANGPLNEERWIYKGRTWIQLTWKSAYAGFGQWCFERGLVNDPNIFVNHPRMLAETRWAGLGAAYYWLTTRRPTRKYPTLNEASDARDVLVATQIINGGTTHLAERQALYNRAIALGDDLLQILGGDEDEMAGWDQTKVDRAMVLLENIAGVRRKSRSPLRWPYEKEIDTAIGFAWTADANVHVQLVEKLAVIYGDPTSIALLTAVANTDEPGREGDRELARRILARCNEDAVQEAHKRINEWLEAEKAHKVGV

SEQ ID NO: 87 - protein, 512 aa, synthetic construct
MALGMTLENGWPECDLSDTERLTIPGTPLSLPIRAGQPHAILQAFFRDMHDFIEPVTNAKGIADEGSWTENNSVYTSNHKGATAVDWNWSDHPVNFMAPDPRAGWNGSVLIPGDQTPAVRELLAWYEGMVYWGNDWSSFRDSMHFQMGYNTCCGGANAARVQSFIDRKIRADGYSTYRRGGVPRGGGPAQAPAAPANPIKPTSGLTAAVLHRIAVKRLGNAAPPLSFYEERIGAFLAMLRDCNANTIDRRAMVGAQLFHEAGALRYVREIASGAAYEGRKDLGNVYPGDGTRFRGRGWIQLTGRAHAAGFSGWMFRRGKAPTATYFVDHPEQMETIENAAQVAVYYLTVSRPGFMALADARNIVAATKAINGGTNGLADRQAWYDLFLAENANLLDGPETPTDPIEELFTMTAPFRSTSHYRTDDEANLTFGHILRSVDAMTHEQMVELAALRGEVWAIQLLAKLANGQLPGAKDPDGAFWVARAVSLLNAIQTTRPDWITAATEAARGGTA

SEQ ID NO: 88 - protein, 321 aa, synthetic construct
MAFIQKQGYWKSENGWRMCDTAELDYTPVPETNFKLGVRKGAPSVILKSLIWRLNRIEPMIVNQIGCYTDENSMRNSNHNSATAIDYNWNLHQYQKWGTWGNNRAAVDKVIADFRGIVEFGGNWTSPRDEMHFELHFAEGHQGTEQLAQELRDGLWGIWKAGTAPSDAPSSGGGSSAGDILRIGSTGPEVLKMQKGMNAVFKNYKAMPLDEDGIFGPMVQAAVIEFQQRSLISVDGEVGPQTKAKLAEYGIVLTGQTTPTSPPAPAAFVYPSHEEMVKQLWEQAFGPQAKGWESLFGKSVDGSRGKFTVEAIADIRNVATQ

SEQ ID NO: 89 - protein, 389 aa, synthetic construct
MTTKDQVAQITIAEAKARSYTRSECLAIMSTFYQESGWNDTIWDPTHTTYGIAQQDGSYPHRFDGAAAQIKGFFDKLDVWRAKPGASTDIWLNICWMQQAPNWPSADYWYANGRRAYLTEIKSRIATVTPYLDKYWPTTGGTDVPETRPPYNEFPIWSENHYNTKRTVNDIDAFLLHTSEGFVGRDDAAEALSLWYQPRSRQVAYHYAVSQASDGGVTVVDNVDTDYASWSALSANGRSINLCFAGTRAAWSRNEWLGKFGNAIDVAAYLAVQDCKKYNIPTKVIAPPYTGRLPGITDHRYVTQILKDGTHTDVGDGFPWDYFAERVNHWAAGGKTEPEPPKVKRFPDDWSDREILVEILRQLRGYNLTGWPQLGGKTLVDAVAELLGH

SEQ ID NO: 90 - protein, 524 aa, synthetic construct
MVTRANVEATKEFIRARLGNPYVYGGALSENVRQGTDCSEVWQTVLEMVHGRWRQGRQAEGATTESYRPRSMGGPIPDGGVGPFGVVLVDHWSKIPANAAAKICFHHGPGGGANSHMWGELDGMLIESGGSKGLVTGDRAMQINSSYANAWAYLPGPIGGTASLGVGSTGPAVISLQTRLNERGANLEVDGEFGPLTADAVFKFQQNNHVVGDPPGVAGEATLRALGLLGNDGAIPAQPDTLAAEYLAAATGLSVARAKQILPAVVDGLKQSSATNVNRIAMWLAQIGHESAGFDATEEYEKGDGGATERWKYLGRTWIQLTWLSNYLGFSKWAFDRGLVPTPTYFGDNPRELAELKWAGLGAAYYWTVARPDINALSDRRDLDTVTRRINGGTNGLTDRRNRYNRALAVGDNLLRIIATTTEPLDEWDALMADQTRYASRAWFRSDNVANLTPLDLLRNIDGMKYDERVVQAAFRGQVWAINAIAALAQGQGPEGQNPVAVAEAQDIIRQLQAALAAQNGASK

SEQ ID NO: 91 - protein, 706 aa, synthetic construct
MQVLGQEITKVRVAGADAARVRIFGEDAWTAEPPTPVPDPVSYFDWHEGSGSTTTSIVSSHVLTAVNPSTAWDGESAAGQFEGDIGDVATSTWSIAVEFTLSAGSSWRNILHTSGAGAADEVWLQLNGSTLSIWTSAGASTGFDPAQPTLPPNLPIQLVMTNDGTTQRVYVDGELVSSRARNNILEPTSAVIMDTSEKLNGYVHSLRFWDVVLSDAEVEALMPDSGDEEDPPLFRGMTDEITQYGITWTFDQEYMAGQFITGDWWVVGPVTVTSVSPAPQGRLDGEDDDDLTGEAGQRVDDRMRHGSMVLTDVTTDQAYDSRCRFYNPARAISYPYNLAANRSLISSVSAVHPGGSEMGDLADGAEGRVMETAAVLTCLDEEPPEDAFRPCYWGADKSKIVRASDIQWDRLANVTPASSPPTLVEYARFFERPWLDHMQAYWVGQFLWPWKNQPGYGRGYAAVVGTAGLLVNCNYTEAQKRPIVHGLIQLGIDLAGMLKADSCFRVSGGHGSGRKFPVVFAHLMLDDDPYFQFEADAVGADVGEDGDGEPQALFGEDASTYWGETYYGAPACFQMVGYSSEVPPHQESPPPYTDWDDTSEGYRQCCTVGAWVGQTLAILLMGAKAAWNHDSYFQVVDDWMADPDPYAANRGGHPRPDDWQETNAWHPWATRMWNLHRSSVPAQDDGTDNYKWMALDREWVPNPPPA

SEQ ID NO: 92 - protein, 438 aa, synthetic construct
MADRFFPMRDGTYALSSGFGARWGTQHRGLDFAAKDGTPIYAAQAGTVAYIGPAQGFGQWIVIDHPAADGAGTTVYGHMWNAFVTGLKAGDRVQAGQLIAYVGANGQSTGPHLHFEVHPTVWRQGSQIDPLPWLHGSRNPGDAPAPAPADPPASAPPGGQRMQDPFTGEVWSPNRSVRQKPAPRWIAIHTQEGGRTARDLCEGWLAKRESQVSYHVAVDDREILKVVAESDRPWAAANANDYAFHVVAAGSYAGWSRGKWLETDASDGKNEDVELTNLAKVCAWWCQKYAIPAEWIGGRGVPWGRDGICGHEDFGAWGGGHHDPGPNFPDDELIRRVRVLLGGSTPEPLPPAPPVALPGTNPDQYAGVLLYRGRPGQDPRQVRVLQTRLKRAYSKLDVDGIFGPHTEACVRDYQHLHPPLVADGIVGPATAAALGLVF

SEQ ID NO: 93 - protein, 491 aa, synthetic construct
MTFVVTRQRAQEVHDRARARAGLPYAYGGAFTNDPKRSTDCSGLVLQTAAWYGGRTDWVGNRYGSTESFRLDHKIVYDLGFKRLPKGGLAALPFKPVMLVGLQHGGGGVYSHTACTLMTMDIPGGPVVQSARGVDWESRGGNPGVRLYDGARAWNDPLFHDFWYLDARLEDGVTAEVDRAEILARATGLTYNKAAVLLPAVRDGLIQADCTNPNRIAMWLAQIGHEADDFNATEEYASGDAYDTRTDLGNTPEVDGDGRLYKGRSWVMITGKDNYRDFSRWAHGRGLVPTPDYFVVHPHELAELKWAGIGAAWYWTVERPDINALSDRRDLETVTRRINGGLTNLEDRRRRYNLALAVGDQLLELIGDDDELADPTIQRFIREIHAAMFNTVVTQSPYGDPRNPDGSEPDSNTWQLHELIKNGDGMGHARYVEESAKAGDLRELARVVRSAKGQGRDKSPEFVARARNVLAQIEAANPEYLEAYIARNGAL

SEQ ID NO: 94 - protein, 518 aa, synthetic construct
MVTRANVEATKEFIRARLGNPYVYGGALSNNVRQGTDCSEVWQTVLEMAHGRWVPGRQAEGATTESYRGIKPGQVGPFGTIAVAHWRDIPANAAARIAFHHGPGGGANSHMWGELDGMRIESGGSKGLVTGDRALAVESTYATNWAYLPGPIGGTASLGIGSTGPAVITLQAKLNTRGAGLEVDGEFGPLTAAAVTRAQQELHVVGDPPGIAGEATLRALGILNDNGAIPANPDTLAAEYLAAATGLSVARAQQILPAARDGLLRSDATNVNRIAMWLAQIGHESAGFNATEEYEKGDGGATERWKYLGRTWIQLTWLSNYMGFSKWCFERGLVPTPTYFGDNPRELADLKWAGLGAAYYWTVARPDINALSDRRDLDTVTRRINGGTNGIEDRRNRYNRALAVGDNLLRIIANSPEPQDEWEALMADQTRYSSRAWFRSDDVANLTPLDLLRNVDAMKYDERVIQAAFRGQVWAINAIATLAKGEGPEGKNPAAVLEAQDIIRQLQAALAAQNGASK

SEQ ID NO: 95 - protein, 454 aa, synthetic construct
MDINIMRQALSPTSASDTWLAECLPHMEEAMRAAQINNPRRASAWYSQIGHESAGLRYMAEIKTSDPSWSWDRTRYRGRGPIQLTWQGNYRKFGQWCKDQGYINDPELFVNQPELVEQPKWGFLAASWYWLCAGPRPGQINGFADVGDIVAVSRCINGWIETKLPNGMPDRQQRWDHCLALGDAIVPGNITPPKEIPVTWTGDPVWLEEVLRPALGDRLKTLPGWQNSGHGDFKDIRGVMVHHTGDSRESAQSIRNGRPDLAGPLANIHIAPDGTVTIVAVGVCWHAGQGSYPWLPTNNANAHMIGIECAWPTPRPDLPKGYDPAERWPDAQIISMRDTCAALALKLELPATRVIGHKEWAGAAQGKWDPGNLDMSWFRGEVRKDMEGFVFPGEHPPIEPQPGPTLPPDYAKETWDQLRIQWPQLGGRTLVDAVAVIGEKLGIEGFYDVKKTQS

SEQ ID NO: 96 - protein, 526 aa, synthetic construct
MVTRANVEATKAFIRARLGNPYVYGGALSENVRQGTDCSEVWQTVLEMVHGRWVQGRQSEGATTESYRYIDVGQVGPFGTIRVNHWRDIPADAAARIAFHHGPGGGANSHMWGELDGMRIESGGSKGLVTGDRAMAVESSYATAWAYLPGPIGGTGSLGVGSTGPAVIALQHKLNERGAGLDVDGEFGPLTASAVTRAQQELHVVGDPPGVAGEATLRALGLLGADGAIPAQPNVLAAEYLAEATGVSVQKAQQMLPTMRDGLKQADATNVNRIAMFYAQTGHESANFNATEEYASGAAYEGRADLGNNYPGDGVRFKGRTWIQITGRHNYGEFSKWAHWKGLVPSPSYFLDHPQELSDLKWAGIGAAWYWTVQRPDINALSDRRDLDTVTRRINGGLNGIDERRTRYNRALAMGDKLLTVFASNEPQDEWDALMADQTRYPSRAWYRSDDVPAHTPLDLLRNIDGMKWDERVTQAAFRGEVWAIEAIAKVAKGEGPEKSPAAIEESKNLVRQLKAALAAQIGASQ

SEQ ID NO: 97 - protein, 253 aa, synthetic construct
MAFIKKQGYWVSENGWPMCDTNELDYGAIPDVGMKLGVRIGEPNAILKALICRLHREVEPAIVSQIGCYTATNSMRNSNHNSATAIDFNWGKHPWKVRGTWGALKQKVDRIVADFRGVVEWGGYWSDSYVDEMHFEMHFAPGHQGTIDLARELWSDGLWDIWKPGVRPPSTPAPPTTVYFLLTIGSTGDTVRKLQAQMKEVFRSYAGHLEVDGIYGPRTAAAVAEFQRRTGIGVDGDVGPITRTTLKKYGVIL

SEQ ID NO: 98 - protein, 455 aa, synthetic construct
MPGSEIPRYWPLGAGRIVTSPFGPRSGGFHAGVDFGRNGGSAGMPVYAVQSGTVIYAGAAQGYGGPDPAGWLVIDSTDAEGSGCLEYGHIVREVSKGQHVSAGQRIGHINPDTRTNGGTPSNPIAPHLHLSDMPREYNPGAKQDPMKRLAGAREPEAAPPAPSTGGNATVALADPITKALWTARNRYSPRGLPSPMWIGCHTSESRSRAVNLRDYCERNEVSYHRIVDDVDIVGMVRDTDGSWSAVGANKYAYHICWSSSFASWSREQWLDPTPGDGFNERNALRLGAKQIAYWIQQSRAAGRPIPVEWIGGRNRPPWGLNGICGHVDFGTWGGGHSDPGPNFPVDTLLSDVREFLTGEPQPPIVAPPPVGVPGTNPDKYADWMLYQGNPRNNVDRVRAVQRRLKYAYAAYAGHLAIDGDFGPLTRMAVEEFQRRSKPLVVDGIVGPMTAAALKP

SEQ ID NO: 99 - protein, 488 aa, synthetic construct
MTLENGWPECDLSDTERLTIPGTALSLPIRKGQPHAILQAFFRDVNEFIEPVMNARGLSDEGSWTENNSVYTSNHKGATAVDWNWSDHPLGVKNGGWDGSVLINGSQVPAMRELLTWYEGMVFWGNDWSSPVDSMHFQMGYNTCCGGDNAKRVDSFIQRKIRADGFSTFRRGGTPRGGGFAELPAAPVHPIKPTSGLTPEVLWRIAGGAASKLPVSHFERWFDELVECQAACGVLGNIDRSAMWYAQVFHESGNLVHTEEIASGAAYEGRCEGLGNCQPGDGVRFKGRSFIQVTGRSNYTKLSGWAHSKGYVPTPDYFVVHPDQLDDEQYAMLGVTWYWTTQRRMNDAADARNLELATRYVNGGTNGLDHRRAIYNRALAENANLLLTNPVEPWEELMATAVPSLSIYANPGEADVPLAVMIAALDAHGPHEPYVERQAQEFGDVDSIRRIARTANGQGRVKTPAAIKQATDAFRLIPPEFVRAAIPA

SEQ ID NO: 100 - protein, 457 aa, synthetic construct
MTAIITRQRAQEVHDRARARKGLPYAYGGAFTNDPRRSTDCSGLVLQTAAWYMGRTDWVGNRYGSTESFRLDHKIVYDLGFKRLPPGGIAALGFTPVMLVGLQHGGGGMYSHTACTLMTMDIPGGPVKVSKRGVDWESHGNRNGVGVDLYDGARAWNDPLFHDFWYLDAKLEDAPAPEDDAVEILSQATGLSTERALEILPAVRDGLIASECVNANRIAMWLAQVGHESASFKYTEEIAKNGRYAPYIGRTWIQITWDYNYRAFSQWCFDRGLVSTPDYFVRNYTDLALLKWAGLGAAWYWTVARTDINALSDRRDLETVTLRINGGHNGIADRRERYDRASALGDRLLALISDAQPIDPFEEEMMREVESFSIYATPGEPKIPLFVMLQSLDAHGPHEPYVEEQARHGDRDAISRVIRTAAGKGKYGTASGPVNQASRVLAELEDSGVLSNYLKGN

SEQ ID NO: 101 - protein, 489 aa, synthetic construct
MITENGWPDCGPNDLDRKPIPGTDVVIPLQKGQPSLILKAFAADLNWYVESVYNSRGGTDEGGWTGTNSVPTSNHLGGTAFDYNWSDHPLGYAAPDPRAGWNGSTIIKGDQTPYVRELLDYYEGMVYWGNDWRSPKDSMHFQMGYNTYGPVGSTTWKRVEDFIARKIDITTGFSRFREHKGTTGVNFTALLSEAMLDVSGVDYAYYVTLVARTLRDCECTTEARIAMWCTQVGHESLSLKYMEEIADGSQYEGRTDLGNTQPGDGKRFKGRGPIQVTGRYNYTKFSEWAHSKGIVSTPDYFVMNPSELSKPEYGFHAVTWYWTVQRPMNQAADARNLEQATKYINGGLNGLPDRKMRYERTTAMGSRLLNLLLGEDDWMSDPTIVKMIKEIHGCLFNQIGSQSRYRADGEGDIWKLHELIKNDDGMVHEAHIERLAVLGDPSSIALVARNSERGDKLAQAVLIRIEDKYLTETQQHLKNTIIAQRFPEG

SEQ ID NO: 102 - protein, 316 aa, synthetic construct
MAFVSRYGNWYSENGWRMCDANELDRGAVPGTSLVLPIRKGVANLILKGWVAWFHRNVESLNNARGFSDEGAWTPTNDVANSNHLSGTAVDLNWSDHAFRVSYSGFTQAEINKVREGLKLFEGTIWWGQDWNSPKDPMHFQLNYGEGDARNAAFATKLQNGYLNIWSGGGGVIEPPAGVDDIYAELGDSGDRVKRLQQFFNDNFKSYSRLEVDGDFGPATKAVVVEFQKRVGVLADGIVGPVTLAAMVKHGFKTESGVKVANLPEDWTDRQVINDIWEQLRGPDGKGWERLGKNAKGENLSLVDAIAEVKKRLEAA

SEQ ID NO: 103 - protein, 427 aa, synthetic construct
MSFTWFADKPLRTREQIAREVHAVSLARGLDELATVIALMTISTEVGTGTGDDRKWWCPANDRVPATKNYPHDSRSDDNRSSGYFQQQPGPNGEPWWGTPENMMTLPQAANTFLERLSDDYRRAANNPRLAGEFAQRVQQSAYPDRYADKWDEAWTVLRRALGSQPPATPEVPMPENRPDFNEFAIWSANNSVRSGKPTMFLIHTQEGGGGDAAAENLAKWFQNSNGVSYHYTISQASDGGVTVVDCVDTDRAAWSVGNANSISINLCFAGSRAAWSRDQWMKQSNAIDVAAYLAVQDAKKYGFAPLVVPPPYTNGRPGISDHRWVTDVFKWGTHTDVGAWFPWDYFAERVAFWANGGVATEPEPPKVKRFPDDWTDRELAVETLRQQRGYTLNGWPQLGGRTVVDVLGAIGAKLGVEGCYDVKGKS

SEQ ID NO: 104 - protein, 498 aa, synthetic construct
MALGGRMENGWPECDLSDCDYATVPGTPLRLPFRKGHPFIILQAFLRDLNEFIEPLMNARGATDEGSWTDNNSVYTSNHKGATAFDYNWSDHPMGNANAGWNGSVLIAGDQVPAVRELLRFYTYRGIQLVWWGNDWNSPKDSMHFQMGYNTVNNPQIVQEFIDKFIRPDGFSTFRRGGAAPSAPVVDAATILARATGIAYAKAQEILPTLRDGLILADCKSVPRIAMFVAQTCWESDHYNATEEYANGPLNEERWIYKGRTWIQLTWKSAYAGFGQWCFERGLVNDPNIFVNHPRMLAETRWAGLGAAYYWLTTRRPTRKYPTLNQASDARDVLVATQIINGGTTHLAERQALYNRAIALGDDLLLILGGDEDEMAGWDQTKVDRAMVLLENIAGVRRKSRSPLRWPYEKEIDTAIGFAWTADANVHVQLVEKLAVIYGDPTSIALLTAVANTDEPGREGDRELARRILARCSEDAVQEAHKRINEWLEAEKAHKVGV

SEQ ID NO: 105 - protein, 459 aa, synthetic construct
MSFIRSAFADKPLLTREQWMKLFIRVADELDMPDRRGAAVLAAMCAFQEAGADPHNTGKRQIWVPGNNADPCFKANPAAYPHDSMGDDGQSTGPFQQQMNKPGTTPWGWGGNYGDPEGTRKRMDPYESTKMFMGHPSSGLKKKGYDASNAQTANDAIQRVQGSGVPWAYAQWWDEANRLYDEVVGDVPPSTGGGKVPVSGDPVWLEEVLRKRLGDRLVVHDGWKEYGTGGVMGEIWGVMIHHTGNRNASWESIRNGRPDLRGPLSQALIAPDGTFHLVAVGPCNHAGVGSYPGLGSDGNRRAIGFECAWPTIRPDGSFDKGERWPDAQILTMRDATRAVLEHLGHDHTHVCGHKEFNKVDGKWDPGNMDMNWFRGEVRKALAGEFDPRAPEPEPQLPSQPGIPTDHDLLVYDQICGRWEMLGWRTPVEALATILDELRGTKNAGSRGFTRGPHDIEDKK

SEQ ID NO: 106 - protein, 520 aa, synthetic construct
MTERVLPFDRKIIRQDTGYWCGPASTQMALSARGKYVDEATLARECKTTVNGTDNVGLIERVLDVRLPEGNYTSMYPGGVKIGSPARPAAERKTRFWWDIVRSIDNGFAVILNWVVPPARKPIRAVKGSTNPSYGGGTTYHYVTAVGWSDEGNNGRPAVLIADSGFAPNVYWVDLDTAFALIHTDLWKGYAYADLPLIAPPPPGVEVPPGIPVKPGTPPVAVTPPVPAPPTQPPPTKLGSLTDPFTGALWSPNHYDGRGGLGTPGWIAVHTQEGGRTARDLALFLANPANEVSYHSVNDDIEVLKCVAETDAPWSASNANKYAFHHCFAGSYAGWSRDKWLSPDASDGKNEDVQLTKGAHVVAWWCDKYGIPAEWIGGRAQPPWGARGILGHVDLGQWGGGHFDPGGNFPVNEFIRRVVTFLTGEVQPPLEPLPPVVQPGTNPDAYSDWMLVRGDPRNDVDRVMRVQSKLKRAYAAYAGHLDVDGIFGPATQAAVREFQRRSNLVADGIVGPMTAAALRP

SEQ ID NO: 107 - protein, 544 aa, synthetic construct
MDVKRANVEGAKAFIRARLGAPYQYGGALSSTNVRQGTDCSEVWQTVLESVFGRYVPGRQSEGATTESYRSVKVGEVGPFGTIRVARPQDIPADAVVKLAFHHEGSGGASSHMWGELDGMLIESASKKGLCTAPAAWPIDHSYANAWAYLPGRIFEDGTAPTVVEPQDTLYADVSEWQVPVTDAYTDAGYRVLCIRSNDGTHRDEDWSNNYAWCKRAVDDGRMAFFIVYFVWRPNWQAAVDTLKTQVGEPHPKMAVMLDVESWNGQIGGDQSAGINAAFEQIAGWLGDRRRVIGYGNTGDLNALWPRKPEGVRLVVAGYGKLPTYPGMIAHQYTDGQGYGGGLPEGAPPFGRCDMNAANGLTASAFARALGIEPSTTGEDDFMSALSADEQREMLTYLRWAFAPGTGEFRKRFPSRSPLRHLGEGLIDTAVGIDLNDDANDHVVLVKELAEIGDPGALALLHEVAGADPVLYPDRQEDRKLAQRILDHLPQSSTKPAEPDVTPAPVRNVSCAQSGAGCILVANGGDGSCALAGDDCVLRKGATA

SEQ ID NO: 108 - protein, 441 aa, synthetic construct
MRFYPLGTGRIVTSAFGATTGRSAPHGGVDFGRAGGSAGMPVYAIQSGTVIYAGAASGYGGPDPAGWLVVDSDDDEGGGCLEYGHIVREVNRGDHVTAGQRIGHINPNNRTNGGVAPHLHVTDWPHAYGQGSRQNVMARLSGAREPEAQAAPTPTEQGSVVGDPVWLADVVRPTVAKFAEYPNWRNRGHGDFKDIRGVMVHHTGGPASAASIANGRPDLAGPLAQLHISRDGTVTVVAAGVAWHAGAGSYPWLPTNMGNWHLIGIECEWPYGEPGINERNAHTVRWRDPEIIALRNTCAAILLRLGLGVDRLIGHKDYAGRAQGKWDPGNMSMDWLRGEVRKDMDGFVFPGEDIVNVTPPPVPAPVPAPPATANVLLHRGMSGPNVVKLQTTLRRWYSKLAVDGDFGPHTEACVRDRQRIYGLDVDGIVGPLTAAKIGLVL

SEQ ID NO: 109 - protein, 452 aa, synthetic construct
MADPALVSSRPVRFYPLGTGRIVTSAFGSTTGRSAPHGGVDFGRAGGSAGMPVYAIQSGTVIYAGAASGYGGPDPAGWLVIDSDDAEGGGCLEYGHIVREVNRGDHVTAGQRIGHINPNNRTNGGVAPHLHVTDWPHAYGQGSRQNVMARLSGAREPEAQAAPTPTEQGTVVGDPVWLADVVRPTVAKFAEYPGWRNRGHGDFKDIRGVMVHHTGGPASAASIANGRPDLAGPLAQLHISRDGTVTVVAVGVAWHAGAGSYPWLPTNMGNWHLIGIECEWPYGEPGINERNAYTVRWRDPEIIALRNTCAAILLRLGLGVDRLIGHKDYAGRAQGKWDPGNMSMDWLRGEVRKDMDGFVFPGEDIVNVTPPPVPAPVPAPPATANVLLHRGMSGPNVVKLQTTLRRWYSKLAVDGDFGPHTEACVRDRQRIYGLDVDGIVGPLTAAKIGLVL

SEQ ID NO: 110 - protein, 459 aa, synthetic construct
MSFIRSAFADKPLLTREQWMKLFIRVADELDMPDRRGAAVLAAMCAFQEAGADPHNTGKRQIWVPGNNADPCFKANPAAYPHDSMGDDGQSTGPFQQQMNKPGTTPWGWGGNYGDPEGTRKRMDPYESTKMFMGHPSSGLKKKGYDASNAQTANDAIQRVQGSGVPWAYAQWWDEANRLYDEVVGDVPPSTGGGKVPVSGDPVWLEEVLRKRLGDRLVVHDGWKEYGTGGVMGEIWGVMIHHTGNRNASWESIRNGRPDLRGPLSQALIAPDGTFHLVAVGPCNHAGVGSYPGLGSDGNRRAIGFECAWPTIRPDGSFDKGERWPDAQILTMRDATRAVLEHLGHDHTHVCGHKEFNKVDGKWDPGNMDMNWFRGEVRKALAGEFDPKAPEPEPQLPSQPGIPTDHDLLVYDQICGRWEMLGWRTPVEALATILDELRGTKNAGSRGFTRGPHDIEDKK

SEQ ID NO: 111 - protein, 377 aa, synthetic construct
MTFSPLISGTINHNNKYSSRQGRPVLKIVQHHWAGTGGGIERMEGPDQASCTYVILTDGRILGHVPEEFRPWTSGSFEADGDAITIEVQNINGRMDGNDNSPNSWQISDAAYASIIKLIADVARRYGWGGVAVGNYRGHREFSSTACPGGFLWARMDNTRAQAHAAFVGGKPAEAPAPVAPTTSGKTVWQLADEVLAGLHGSGDARKNSLGAQYDAVQAEVNRRFGVGLPATKAPKSVSQLADEVLAGKHGSGDARKASLGSRYAEVQNEINRRLGGGGVAPQGVNIGALADAVLRGEFGSGDDRKAKLGVNYDAVQAEVNRRLGGGVAAAPPARTVNIGALADAVIRGEYGSGEDRKKRLGSNYAAVQAEVNRRFK

SEQ ID NO: 112 - protein, 211 aa, synthetic construct
MNGAQVIGAIAKRNVELGGRSVYTRGWETRGNGQSPAYQGLLLHHTAGGRNVNIDQILIDGRWDLPGPLCNFCIMYDGDLGVISANPANHAGASGGWDTAPFARTGLFNRQIIGCEIQYPGVEPMSNAQYETAKRLSIATLEVLGKPRDTRWIKFHQGTSIEGKWDPGYANGKTYNISEFRKDVLGMRPTGEDENSAAWLDNYRQLFGYPE

SEQ ID NO: 113 - protein, 320 aa, synthetic construct
MVAPVIKMPNTEKHSGVSGIPTQILVLHSGECPLRGGYAQSLTQWGNVPLAQGGPEASWHWFVDPIAIVSMVDPQFAAWHASEANPMSEGFEQGGYARFTRAEWLTPEGVKQMDNLAWIMAQRAKANGIPAVWLTTDQVTAVTTFGNRTIKGFCLHRQIDPETRTDPGDGYPYDLLMAKVKAYMSGSTFTPQSTSTAKPQGLFMALTAAQEKTILTRNEKYVDAPISQVDEKAATAVWETKVDRGAKSVSALQELADCKSMLLAQQGVLAGLTNAIQQLATKPGSPVDLDAIGKAAEAGAAAALADLTATVTIKQEATNG

SEQ ID NO: 114 - protein, 205 aa, synthetic construct
MRPVAAEFETSQRFGEGATQGVVANSDPNSGMGYYVWLYGNYQPFGHAGEDEACPIGTPVHAIADGTVVWAGWAEDMAGDDSDWGYRQRFYVYKRFPGILTVIRHDNRADGLLYTLYGHLSNNDPAPAGTVVREGDLIAYSGNTKSKTETLEPHLHVEALVDMSYRSGGGLIYGRVDPRQFFGTTTPTTHTADELFLLDLGLSIP

SEQ ID NO: 115 - protein, 295 aa, synthetic construct
MTALVRPHKGPHTQRFGNLQPDGQPHAGDDFGYTDGVNVYPEVYAAAAGEVIYAGDARALGWPNPFYLNPDFDRTDAQDSSAGNVVVLDHGDGMTTYNHLESIQVRKGARVAQGQRIATTGNTGFSFGKHLHFEWIPYPANFGTATYGRARPTFVTVTAQSTKPKELFTVSQYAALNAKLDTLIEATKPINTASGKKTLRDFVAAGTRAAEAARDNTAPINVTGGQESLRAFVAKGTRAAQANAARLAALEAALKAVLENPSGVSLEAVTDAAAAGAERALANLTGTIELESKNG

SEQ ID NO: 116 - protein, 312 aa, synthetic construct
MSTGFTLPTSRPVTQPWAAEFDDWDGDGIVDIPGGFYHSIGWFGHNGIDFGCFEGDPVEAVADGTVIFAGDAANHYLLSGGGNVAFIHHEFYGVWTESLHMSRVIVANGQRVTKGQVIGYAGSTGSSTASHLHLGMFASEFPNQWDGWRGRIDPTPYLYGALNSDYAIKTHTAQIKEAEMPAVLHKVVSPAMNRRLAKGKAAVITTDATNTAHQNFAVGGVGTYDIQAYVAGTGLPDGQRITGRFLLVEKGKAPSGYYPFQIDGTFDGKFNGLIGGRFKVNAGTVIWIELTSTVDTSYVASVDGSVLVHPFK

SEQ ID NO: 117 - protein, 606 aa, synthetic construct
MTTVIDTAGGFPSPEAIKAAGHSGLMAYVSLSRPGSNFAGKPITRAIADRYRAAGVDIGAIFQYGKPQGTAPSDWTTGFEGGKRMAAQALRIARDAGMPGWCPIYFAVDENITLQQWNDTAVHFFRGAGEAIGVEWVGIYGHSRVCHWAIEDKVVGRSPKPGSGPWAWVTRAWSDDTGTGYASLYQRVIDTPSNPGPLVDGIRVDVNDVYAADWGQWSVDRTPNAGQPAPAPTAPGGPADMARIGYGVTHTIPAGSDGPRRADDYVGVHTQEGGKGDAIGLANYCKTAGVSYNDAVDDVYTVRMMPPGNAPWAAVQANAVGYHVVLAGSFVSWSRDRWLSKDASDGLDEDAMLTRAARCVAAACQEFGIPVEWVGNNGATGWPQKRGICGHKDFGARGGGHTDPYPNFPIDEFMRRVRAFFAPPSPNLIDAEAKVAARWIGKRITGAATPTTTDDETPLFLDGKKIGAFARFESGHVYWRLGAALAYAIPAGGLFEAYAARDWERGLGFPVLRHTVVTTPASKVTAGVQSFEGGVLFTPVGGPVEGFVVHGEIGKRYAAMDWEQGPLGLPTSDERPVPNTDLIEQRFEFGKLTYVPTGVLVELVTN

SEQ ID NO: 118 - protein, 366 aa, synthetic construct
MADPVWLPDVLRAEGLRCDIYPGAFERGHGDFGVIWGPFMHHTGSFGETPRGIAQHSSLGLASQLHLAANGVVTLCGVGVAWHAGTGSWSGIPKNNGNAVTIGIEAAHNGTAAWSDAQYGAYLKVVRAINKRLGNPWNKVVAHKEYGAIQGKWDPGNLDMKLFRQRLLQAPDKPLVVMNMIELEAKENPWVGVRKAKAGADGERSIGRDGKGRFVEYDNAHIYFHPATGAHAIPHGGLFEAYTERKWETGELGFPVRDFTKLADGAVMAFQGGVLYRKDGMGHHVVKGVIGQRWALEGYEKGPLGWPTSDEIPNGTGGKYQTFEHGVLEWDPSGAVKKIGDAAKDLTLVNAAGIPLAVEAVDLIAA

SEQ ID NO: 119 - protein, 270 aa, synthetic construct
MIMTDLANHPGFWLRDDAAKAFDAWEAKYGKRKLNSAGRTKASQQGLINRWHQGGPANRPPYLYRPAEPAETSPHVIDGGIAVDLEDWREAKKTSEEFGFHWYGDGDVVHFNFRGWDGNSAKRGGYQDGTVELKRFQEKLIRMGHDLGPTGADAVFGPRTKAATLHEQGMAEKNGYPGGKVSDDGLPGPQTEAYLDWWLVGRHAARPSSKSAGELTYADIQAALRRHGYDLVVDNVWGPKSSAALADFQRKSGLKVDRLVGPLTWDKLNR

SEQ ID NO: 120 - protein, 249 aa, synthetic construct
MAYNYVWIDGHRVEVAVAYAFRRLKAAFEQAFPGLTLRVRSGVRTREEQERLYYGWIHRLPGFNLAAPPGKSNHEESGPIGPRAIDIYDTGGDAGVTTMNTRRDRWMAANAPNYGFSNAGYNFWPREAWHKEFTGKLGVVPPNWVGPTPYPVSLKEDDMPVQIRNVETKMIAMVAPKFFAYIRDRETTEVTKNVFSVVDEQHQLSNADFFRVTRTMGIPDAEVREGNLWYGAPTQPVAVASPPLIVKKV

SEQ ID NO: 121 - protein, 301 aa, synthetic construct
MAEYVYINGARLTAWMAYILGVLSNDLYRTFGVWLVITSAIRTYAEQERIFRERYVTAGNINGRRVYDTRVWNGVRWYRISSAGTVAVPGTSNHEIQGTKAAVDLADTGRDGGVATAGSVRSNWLRARAHLYGMVASGFGFGEAWHYDILNIFQTPPSSGAGNVTPTPKEDDMTVAILLNGRHYYTAGEEFLSHNGSKGQADITRQVNSAQDELHKLTTAQFYDYLDGMGIPRSVVDVNGGGVLNPQSGKIEGNGVWSRRREAVALAEQYAASLAEFVKRNDAAMAALSKAVAALPKAPAS

SEQ ID NO: 122 - protein, 564 aa, synthetic construct
MGYLAPANVPISDTRAGHIRRGSKEPGTDYATPYGTDLIAPGDGVILSVDPNPGGAEGRRISFLMDDTGEVIDWIHLSEIMMRAGQRIGRGTKGIAKSGASGFGKDWYYGPHVHVTRRARQGLPYSQTLDFQAAVEGGGGGGSAPASADVAKGQRTAGGNGVRRRKQPTSKSAENGEMLKPGTVGNFTGWIRGEKVDGIDTWFQGISGDWFWAGGFTQGANTTGLKDLNPKTPAPKPDPKPTSTQRKVGPNGARRRESPSSKAAEAGEMLAPGTIGNFNGWARGEKVEGNDVWFRGTSGDWFWSGGFEGGPVTAGLPEISIAPPVTPPKPVDPKPTPNADNPRNLPVYKPVYPGAKIGLVAPLTMPRGEKGIPPVKVENKIDIVQEHWTGVTADQLDWFSTDNSRGSCPNWFIRPDGTVYELIRPGMKPALAGPEWNWRSLGWEIQMVEGGKGTPEQFEAVCQLLAWIHSYDGKVLDGTPVTFPLDRDHFKAHRELVATECPGDWWFSQMDAQLARARVIYAEKYAPEQPEEPDTGDLLLARAKALIEQNNALLEEIVAWAEGA

SEQ ID NO: 123 - protein, 322 aa, synthetic construct
MADVKGGYLRPTPAGGISSSWQAHKDRRPPAFPGPSQEPGTDIACAYGTTLVAPEDGVVVEIKTSNSGATGRFITIDLNDGRRVRYLHLSAVLVTSGKRVTRGQAVARSGASANGRDWGVGAHVHVTLWDFHRYVFGPNGTMDFMPQIGPDNDGGAPGLSYSQEVANQQAWLNQARGEKLVIDGYLGGMTRAAIARYQTFLGVKSDGIWGANTQAAHQKYYDKVTAPAPTQGTLVTMDHIGTIGNIEGLQKIARLYGYKGRIDNIWGPGTRQGLANFLNQNYGGRLTNWLRAKWGYTDADDYWGPNMKAALQRANAANKAAL

SEQ ID NO: 124 - protein, 313 aa, synthetic construct
MGYLVPASAPISCSWQCHRDRPKPSTEPGTDHATAYGTSIRAAGAGTIVDSSTSTSGGTGRYVTIDLDDGRRVRYLHLSQVLKRSGRVNAGDIIAKSGASGFGKEWGYGAHVHTTLFPKHAYVFGTNATLDFMKYVGNSTPVTRDQTVANQQNFLNVAQGEKLVEDGILGGETKAAIGRYQTYLRGRGWYNGESDGIWGAGTQAGHEKRYAEWVAATQAPPSPQYHNVTLDDIASIGNVEGLQKIARLYVPQKVDNQWGPNSKKGLQQFLNVNYGGSLVNWLRSKWGYVGNDQFGPVMKAALQRANDANKRAL

SEQ ID NO: 125 - protein, 433 aa, synthetic construct
MTAVVTRRQAQWVHDMARARNGLPYAYGGAFTNDPKRSTDCSGLVLQTAAWYMGRTDWVGNRYGSTESFRLDHKIVYDLGFRRMPPGGPAALPFKPVMLVGLMHGGGGENSHTACTLMTMDIPGGPVKMSDRGVDWESHGNRNGVGVDLYDNARAWNDPLFHDFWYLDAKLEDAPAAPGEMTVPLIAKANGRWGSSSPAWDHLIMRESSGDPTIIQQIIDVNSGGNEAEGLFQITPKTWRAHNGTQFATSARFATPQQQAIVAARIFTKNPTGSDWGAGLPGREDPKQLAAGLVPTEPIGEDGFLSALNDAEQREVLNLLRWIAAPEYGELRKLFQNEDMYRTDNVRRRTAVGVALDARTFSWEDRVEKSAERGEQWAIDLVVKAARGESAGVIRPGSNTPDPFLVNHAKQVLADVERINPEALKIYLRGGSA

SEQ ID NO: 126 - protein, 346 aa, synthetic construct
MTKIITPDKVREVDAMARARDGLYYGYGEAFTRNPKQSTDCSGLVLQTGAWFAGRTDWVGNRYGSTESFRLDYKIVYDLWFKRMPRGGLSALPFKPIMLVGLQHGGGGIYSHTACTLMGMDRPGGEVKQSARGVDWESQGNGVFYYEGARAWNDSLFHDFWYLDAKLEIAPPVNEINAEYDRAKGWIGKRIDVNEQPAPDGEGKFIRCERGHIYFHPKVNTGAPAGMRAIAIPADIFEVWKGQGFERGPLGYPTVRHYTDTGVGTIQAFQGGAIYRKYGTSGGVVVGDIGRRYAALKAEKGPWGYPLGSEKFRNGGQRVQTFEHYDAYWHPSKVIDFLRDPEAPNQ

SEQ ID NO: 127 - protein, 384 aa, synthetic construct
MTTKDQVAQITIAEAKARGYTRSECLAIMSTFYQESGWNDTIWDPTHTTYGIAQQDGSYPHRFDGAAAQIKGFFDKLDVWRAKPGASTDIWLNICWMQQAPNWPSADYWYANGRRAYLTEIKSRIATVTPYLDKYWPADGGTAVPDEPRPDFNEFPIWSNNNSARSGKPTMFLIHTQEGGGGDAAAENLAKWFQNGNGVSYHYTISQASDGGVTVVDCVDTDRAAWSVGNANSISINLCFAGSRASWMRDQWMKQSNAIDVAAYLAVQDAKKYGFTPLVVPPPYTNGRPGISDHRWVTDVFKWGTHTDVGDWFPWDYFAERVNHWANGGKTEPEPPKVKRFPDDWSDREILVEILRQLRGYNLTGWPQLGGKTLVDAVAELLGH

SEQ ID NO: 128 - protein, 390 aa, synthetic construct
MTTKDQVAQITIAEAKARGYTRSECLAIMSTFYQESGWNDTIWDPTHTTYGIAQQDGSYPHRFDGAAAQIKGFFDKLDVWRAKPGASTDIWLNICWMQQAPNWPSADYWYANGRRAYLTEIKSRIATVTPYLDKYWPADGGTAVPDEPRPDFNEFPIWSNNNSARSGKPTMFLIHTQEGGGGDAAAENLAKWFQNGNGVSYHYTISQASDGGVTVVDCVDTDRAAWSVGNANSISINLCFAGSRASWMRDQWMKQSNAIDVAAYLAVQDAKKYGFTPLVVPPPYTNGRPGISDHRWVTDVFKWGTHTDVGDWFPWDYFAERVNHWANGGKTEPEPPKVKRFPDDWSDREILVEILRQLRGYNLTGWPQLGGKTLVDAVAELLGHHHHHHH

SEQ ID NO: 129 - protein, 752 aa, synthetic construct
MGAAVLAVLMAAALGAAPNAPAYSRDGLPIETLEVPSPAMGRNIRVQFQGGGPHSVYLLDGLRAQEDANGWDINTAAFEWFYESGVSVVMPVGGQSSFYADWYQPAANNSGAVTYKWETFLTQELPAWLAANRGQDPRGNAVVGLSMSGGAALNLATWYPTQFIFASSLSGYLNPSAGLWPTLIGFAMRDAGGFNPQHMWGPTSDVAWRRNDPMVNVGRLAANGTALWIYCGGGVASELDTGHDFGGNFSAGYLENITLSTNKEFQQKYLAAGGHNAVFNFPPKGTHSWGYWGAQLQAMKPDLLRVLGVGVAPPPPPPPALPVPAPRRTATARAAGSLTPRGRIDERPSNRGCRGCGRDRSGRAVRLSRGHPTRRGRRPHRPRQGRHHVGGVHHHPRRAADHTAGPGTPDAPGLQQRLAHPSRPRILGDHRRDGPVADRRRLHRRSLRCGSVRVHPRRAGAPRRQRCAPRRRRRRHLHRARGRTRARRLTRRVRQIAAWTGDPVWLADVLRAEGLTVVEHPGWLHRGHGDFRDIRGVMVHHTGADHATAASIADGRPELPGPLAQVHIALDGTVTTVAAGVAWHAGAGSHPDLPTDRANWHTIGVECANSGTGPTAPHRANWPDAQYAALVGTCAAVSRRLGVGAAGIVGHREYAGRAQGKWDPGAIDVDLLRRDVAACVPLAPGCRSEHVRRLQRRLRDAYRGYAGHLAVDGSYGPQTEAAVREFQLRTPGLRADGVVGPATAVALRLAGG

SEQ ID NO: 130 - protein, 758 aa, synthetic construct
MGAAVLAVLMAAALGAAPNAPAYSRDGLPIETLEVPSPAMGRNIRVQFQGGGPHSVYLLDGLRAQEDANGWDINTAAFEWFYESGVSVVMPVGGQSSFYADWYQPAANNSGAVTYKWETFLTQELPAWLAANRGQDPRGNAVVGLSMSGGAALNLATWYPTQFIFASSLSGYLNPSAGLWPTLIGFAMRDAGGFNPQHMWGPTSDVAWRRNDPMVNVGRLAANGTALWIYCGGGVASELDTGHDFGGNFSAGYLENITLSTNKEFQQKYLAAGGHNAVFNFPPKGTHSWGYWGAQLQAMKPDLLRVLGVGVAPPPPPPPALPVPAPRRTATARAAGSLTPRGRIDERPSNRGCRGCGRDRSGRAVRLSRGHPTRRGRRPHRPRQGRHHVGGVHHHPRRAADHTAGPGTPDAPGLQQRLAHPSRPRILGDHRRDGPVADRRRLHRRSLRCGSVRVHPRRAGAPRRQRCAPRRRRRRHLHRARGRTRARRLTRRVRQIAAWTGDPVWLADVLRAEGLTVVEHPGWLHRGHGDFRDIRGVMVHHTGADHATAASIADGRPELPGPLAQVHIALDGTVTTVAAGVAWHAGAGSHPDLPTDRANWHTIGVECANSGTGPTAPHRANWPDAQYAALVGTCAAVSRRLGVGAAGIVGHREYAGRAQGKWDPGAIDVDLLRRDVAACVPLAPGCRSEHVRRLQRRLRDAYRGYAGHLAVDGSYGPQTEAAVREFQLRTPGLRADGVVGPATAVALRLAGGHHHHHH

SEQ ID NO: 131 - protein, 316 aa, synthetic construct
MTENGWPACGPELLDRSAVPGTSIVIPLQRGIPSRIMKAFAADFHAYAESLYNARGGTDEGGWTPTNSVATSNHLGGTAMDLNWSDHPMGKALDGYTAAETAVVRELLAFYEGMIFWGNDWNTPKDSMHFQMGYNTYTNQAKCNDFIKRKIRVDGFSTFRRGGTGGGTTPAPTPTENIYAQLGDNNDRVSSLQRFMNNNFASYSALDVDGDFGPATEEVIKNFQGRVGVAADGIVGPVTLAKLVEHGYVPLGTISTPPPAAAFAYPAHEEMVKQLWEQAFGPQAKGWPDLFGKLADGSRGKYPVEAIADLHADAGL

SEQ ID NO: 132 - protein, 322 aa, synthetic construct
MTENGWPACGPELLDRSAVPGTSIVIPLQRGIPSRIMKAFAADFHAYAESLYNARGGTDEGGWTPTNSVATSNHLGGTAMDLNWSDHPMGKALDGYTAAETAVVRELLAFYEGMIFWGNDWNTPKDSMHFQMGYNTYTNQAKCNDFIKRKIRVDGFSTFRRGGTGGGTTPAPTPTENIYAQLGDNNDRVSSLQRFMNNNFASYSALDVDGDFGPATEEVIKNFQGRVGVAADGIVGPVTLAKLVEHGYVPLGTISTPPPAAAFAYPAHEEMVKQLWEQAFGPQAKGWPDLFGKLADGSRGKYPVEAIADLHADAGLHHHHHH

SEQ ID NO: 133 - protein, 489 aa, synthetic construct
MITENGWPSCSIAECDTNPIPGTDVRIPLQRGIPNIILKAFAANLNSEIESVYNARGGTDEGGWTPTNSVATSNHLSGTAFDYNWTDHPMGPEADDPAAGWKGSSLIRGDQVPAIRELLRFFTYKGVQLVFWGNDWSTPKDSMHFQMGYGTYANQDLCREFIAKFIRADGFSTYRRGSSGGSWNAQVLAEATGLTIARAAEILPQVAEGLRLSECVSPRRIAMWLAQIGHESDNFNATEEYEKGDGGVTERWKYLGRTWIQITWLENYQGFSRWAYQKGIIPTPTYFVDRPKELAELQYAGIGPAWYWTVARANINALCDRGDLNGVTYLINGGYNGLSERQTRYNRAIALGDRLLELLQEGDDMAQVPQDQLDRVFQEQTQEHESLSGYRDPDEGNIGTWCRIDRNKDLMIHELFTEWKAVQAGDLDSIRRLVRSAAGLGANTTPAFIANAKRMLKKVPAEYLQEGLAYLESTYPELLQAFISQNGAS

SEQ ID NO: 134 - protein, 495 aa, synthetic construct
MITENGWPSCSIAECDTNPIPGTDVRIPLQRGIPNIILKAFAANLNSEIESVYNARGGTDEGGWTPTNSVATSNHLSGTAFDYNWTDHPMGPEADDPAAGWKGSSLIRGDQVPAIRELLRFFTYKGVQLVFWGNDWSTPKDSMHFQMGYGTYANQDLCREFIAKFIRADGFSTYRRGSSGGSWNAQVLAEATGLTIARAAEILPQVAEGLRLSECVSPRRIAMWLAQIGHESDNFNATEEYEKGDGGVTERWKYLGRTWIQITWLENYQGFSRWAYQKGIIPTPTYFVDRPKELAELQYAGIGPAWYWTVARANINALCDRGDLNGVTYLINGGYNGLSERQTRYNRAIALGDRLLELLQEGDDMAQVPQDQLDRVFQEQTQEHESLSGYRDPDEGNIGTWCRIDRNKDLMIHELFTEWKAVQAGDLDSIRRLVRSAAGLGANTTPAFIANAKRMLKKVPAEYLQEGLAYLESTYPELLQAFISQNGASHHHHHH

SEQ ID NO: 135 - protein, 432 aa, synthetic construct
MPVRYWPLGAGRIITSPFGPRDGGFHAGTDFGRAGGSAGMTVYAVQAGTVIYAGAAQGYGGPDPAGWLVIDSNDAEGGGCLEYGHIVRRPEIRVGTHVEAGQPIAQINPNSATNGGVAPHLHLSDMPGAYVPNAKQDPMPRLAGALEPESNSTPTQTEVPMSDPTWLPDVLRAAGLQCDIYPGAFDRGHGDFGEIWGVVCHHTGSFGETPRGIAEHPTLGLASQLYLSPDGKFTLCGVGIAWHAGAGSYPGLPDNNANFRTIGIEGANDGGGTPGKPHHQPWSNVQYDAYVAGVGAILKHLGQPASHSIDHKEWAGAAQGKWDRGGIDPNLFRNDVTAWSGGTLPPPPATPVLVPGVPVEYANFGVIRRGDKGIRVVSLQTRLKRNYSKLVVDGDFGPDTEAKVRDYQSLHPPLVVDGQVGPATAAMLKLIG

SEQ ID NO: 136 - protein, 438 aa, synthetic construct
MPVRYWPLGAGRIITSPFGPRDGGFHAGTDFGRAGGSAGMTVYAVQAGTVIYAGAAQGYGGPDPAGWLVIDSNDAEGGGCLEYGHIVRRPEIRVGTHVEAGQPIAQINPNSATNGGVAPHLHLSDMPGAYVPNAKQDPMPRLAGALEPESNSTPTQTEVPMSDPTWLPDVLRAAGLQCDIYPGAFDRGHGDFGEIWGVVCHHTGSFGETPRGIAEHPTLGLASQLYLSPDGKFTLCGVGIAWHAGAGSYPGLPDNNANFRTIGIEGANDGGGTPGKPHHQPWSNVQYDAYVAGVGAILKHLGQPASHSIDHKEWAGAAQGKWDRGGIDPNLFRNDVTAWSGGTLPPPPATPVLVPGVPVEYANFGVIRRGDKGIRVVSLQTRLKRNYSKLVVDGDFGPDTEAKVRDYQSLHPPLVVDGQVGPATAAMLKLIGHHHHHH

SEQ ID NO: 137 - protein, 444 aa, synthetic construct
MDVATLRRAMSPTSVSDSVLAQYLPHYVEAMKAAEITTVRRAAAFASQVGHESAGLRYMAEIATDGPDWSWDRTRYRGRGPIQLTWQGNYRKFGQWCKAKGYVTDSELFVNQPELVEQPKWGFLAASWYWLNGGPRPGQINGFADQGDILAVSRCVNGWVDNRLPNGWNDDDNSRLPRWNRCLAIGDALLTDQPAEETPVTWTGDPVWLEDVLHPALGDRLEALGGWRDRGHGDFKDIRGVMVHHTGNRNATAESIRDGRPDLPGPLSNIHIATDGTVTIVAVGVCWHAGIGSYPWLPTNNANWHMIGIECAHDGNEQWPDAQIIALRDTCAALTVKLEQPASHVIGHKEYAGAAQGKWDPGNMDMNWFRGEVQKDMDGFVFPGEHPPIDPAPGPIVPPDYGKEVWDQLRIEWPQLGGRTLVDTVAAIGAKLGIEGCYDTKAGA

SEQ ID NO: 138 - protein, 450 aa, synthetic construct
MDVATLRRAMSPTSVSDSVLAQYLPHYVEAMKAAEITTVRRAAAFASQVGHESAGLRYMAEIATDGPDWSWDRTRYRGRGPIQLTWQGNYRKFGQWCKAKGYVTDSELFVNQPELVEQPKWGFLAASWYWLNGGPRPGQINGFADQGDILAVSRCVNGWVDNRLPNGWNDDDNSRLPRWNRCLAIGDALLTDQPAEETPVTWTGDPVWLEDVLHPALGDRLEALGGWRDRGHGDFKDIRGVMVHHTGNRNATAESIRDGRPDLPGPLSNIHIATDGTVTIVAVGVCWHAGIGSYPWLPTNNANWHMIGIECAHDGNEQWPDAQIIALRDTCAALTVKLEQPASHVIGHKEYAGAAQGKWDPGNMDMNWFRGEVQKDMDGFVFPGEHPPIDPAPGPIVPPDYGKEVWDQLRIEWPQLGGRTLVDTVAAIGAKLGIEGCYDTKAGAHHHHHH

SEQ ID NO: 139 - protein, 406 aa, synthetic construct
MPTVCAVPRFLPLKTGSFRWGSGFGPRWGTVHLGQDFEAADGTPIYAAQGGEVAYIGAATGFGQWIVIDHPAAEGAGTTVYGHMWNAYATGLKKGDRVSAGQLIGYVGANGQSTGPHLHFEVHPTAWRAGSQIDPRPWLAGALNPGGNVGVSGDPIWLEDVLRPALGDRLRTLPGWKTDGVGGTMGTIWGVVWHHTGNAAEKPESISKGRPDLPGPLAQIHIAPDGIVTIVAVGPCNHAGRGSWPGLPTDNANAYTIGIECAYPRDTSLTEETITREPWPAAQIISMRDVGAALTKHLQVPVSHNLSHAEWARFGPAGVRQYKWDPGNLDMNWFRGEIQKDIDGYQFPGETPTTTLPSSPDYPREIWDQLRIAWPQLGARTLVDAVAVIGKQLGIADFAPIGKDAS

SEQ ID NO: 140 - protein, 412 aa, synthetic construct
MPTVCAVPRFLPLKTGSFRWGSGFGPRWGTVHLGQDFEAADGTPIYAAQGGEVAYIGAATGFGQWIVIDHPAAEGAGTTVYGHMWNAYATGLKKGDRVSAGQLIGYVGANGQSTGPHLHFEVHPTAWRAGSQIDPRPWLAGALNPGGNVGVSGDPIWLEDVLRPALGDRLRTLPGWKTDGVGGTMGTIWGVVWHHTGNAAEKPESISKGRPDLPGPLAQIHIAPDGIVTIVAVGPCNHAGRGSWPGLPTDNANAYTIGIECAYPRDTSLTEETITREPWPAAQIISMRDVGAALTKHLQVPVSHNLSHAEWARFGPAGVRQYKWDPGNLDMNWFRGEIQKDIDGYQFPGETPTTTLPSSPDYPREIWDQLRIAWPQLGARTLVDAVAVIGKQLGIADFAPIGKDASHHHHHH

SEQ ID NO: 141 - protein, 408 aa, synthetic construct
MHRGIDFEAADGTPIYAAGSGIVAHIGPAQGFGQWIVIRHDSGESTVYGHMWDAHALGLRQGDRVFAGQHIAFVGSNGQSSGPHLHFEVHPGAWRAGSQIDPRPWLNGAMDPRTRGGTVSGWTGDPVWLADVLKADGEIDVRELGGWRDRGHGDFKDIRGIMVHHTGGPASAESIRDGRPDLRGPLSQLHISRDGVVSVVAAGVSWHAGTGSLPWIPANMGNWHLIGIECEWPYRGGIGEHNQRDEPWRRPQILAIRNTCAAILRKLAFGADRITTHKEYAGRSQGKWDPGNMDPNWLRDEIAKDLRGVKFPGEGTPDYTPPPTPVPVPPVDQYAGVLLYRGMRGPAVVKLQQRLKDTIVSKLVVDGDFGPKTEAAVRAFQSSKWRRPPLVADGVVGPATAAQLGLVL

SEQ ID NO: 142 - protein, 414 aa, synthetic construct
MHRGIDFEAADGTPIYAAGSGIVAHIGPAQGFGQWIVIRHDSGESTVYGHMWDAHALGLRQGDRVFAGQHIAFVGSNGQSSGPHLHFEVHPGAWRAGSQIDPRPWLNGAMDPRTRGGTVSGWTGDPVWLADVLKADGEIDVRELGGWRDRGHGDFKDIRGIMVHHTGGPASAESIRDGRPDLRGPLSQLHISRDGVVSVVAAGVSWHAGTGSLPWIPANMGNWHLIGIECEWPYRGGIGEHNQRDEPWRRPQILAIRNTCAAILRKLAFGADRITTHKEYAGRSQGKWDPGNMDPNWLRDEIAKDLRGVKFPGEGTPDYTPPPTPVPVPPVDQYAGVLLYRGMRGPAVVKLQQRLKDTIVSKLVVDGDFGPKTEAAVRAFQSSKWRRPPLVADGVVGPATAAQLGLVLHHHHHH

SEQ ID NO: 143 - protein, 684 aa, synthetic construct
MTMVGGFDVHRQQITFDYVDDDGLVRWGQIRPATLSTLRRWLSERCPDGDAEFALEGCTGWRYVSEELAAAGVRVHVGDPAEIAALRGRKKRAKTDRADARLLRTLLLQGRFPESWIPPAHVLEIGNLGRLYCTLMDERRAWQQRIHAQLFHQGCPPVKALLSARGREALASAPLSPAGRQYVDAALRRIDELTPEIDALRTQLVDFARRQAGCRALQRHYGIGWLCAAIMWAEIGDARRFTSSDQLVRDAGLDVTVHSSDAKRSPGHLSRQGSPDSAATHPRQPARRAPHQSSCHRTSSVDPDKAGRRHARLSSRAPHSRTGGSRWLELQRLSLVPPDREYAADPPRPQGRSSSLRCGRSTLTPTPLHRPMPGMGNEAKPQNNDQRIPTFTGVPDTDKDSSMPQTGTWTGDPVWLVDVLRAEGVNPIEYPGWRDRGHGDFKDIRGVMVHHTGSDSASAASIAQGRPDLAGPLSQLHIARDGTVTVVAVGVAWHAGVGMYPWLPTNMGNWHLIGIECANTGTSPTAPHRQNWPDAQYFAMVNCCAAINRRLAQTSARTIGHKEYAGRAQGKWDPGAIDMDILRRDIQDQIGRVAKPAPTPRPPLPVGEYADILLFRGSEGPQVAQLQRRLNEQGAGLVVDGIFGPNTEAAVREFQRRTRGLKVDGIVGPATAAAPRLKVGPGPE

SEQ ID NO: 144 - protein, 690 aa, synthetic construct
MTMVGGFDVHRQQITFDYVDDDGLVRWGQIRPATLSTLRRWLSERCPDGDAEFALEGCTGWRYVSEELAAAGVRVHVGDPAEIAALRGRKKRAKTDRADARLLRTLLLQGRFPESWIPPAHVLEIGNLGRLYCTLMDERRAWQQRIHAQLFHQGCPPVKALLSARGREALASAPLSPAGRQYVDAALRRIDELTPEIDALRTQLVDFARRQAGCRALQRHYGIGWLCAAIMWAEIGDARRFTSSDQLVRDAGLDVTVHSSDAKRSPGHLSRQGSPDSAATHPRQPARRAPHQSSCHRTSSVDPDKAGRRHARLSSRAPHSRTGGSRWLELQRLSLVPPDREYAADPPRPQGRSSSLRCGRSTLTPTPLHRPMPGMGNEAKPQNNDQRIPTFTGVPDTDKDSSMPQTGTWTGDPVWLVDVLRAEGVNPIEYPGWRDRGHGDFKDIRGVMVHHTGSDSASAASIAQGRPDLAGPLSQLHIARDGTVTVVAVGVAWHAGVGMYPWLPTNMGNWHLIGIECANTGTSPTAPHRQNWPDAQYFAMVNCCAAINRRLAQTSARTIGHKEYAGRAQGKWDPGAIDMDILRRDIQDQIGRVAKPAPTPRPPLPVGEYADILLFRGSEGPQVAQLQRRLNEQGAGLVVDGIFGPNTEAAVREFQRRTRGLKVDGIVGPATAAAPRLKVGPGPEHHHHHH

SEQ ID NO: 145 - protein, 321 aa, synthetic construct
MSFINKQGYWISENGWRMCDTAELDYTAVPGTSFNLGVRKGAPNIILKAAIARLHREVEPMITSQIGCYTGTNSMPNSNHNSATAFDYNWNKHPYQEWGTWGANRAKVDRIVADFRGILEFGGNWTSPRDEMHFELHFAEGHAGTEQLATDLRNGLWGIWAPGQAPEPGAPSTPIPDGYLQIGSEGDQVRKLQVGMNKVFPNYRAMPLDEDGIFGPMTEAAVTEFQQRSLIGADGIVGPETKARLATYGIVLDGASAPTSPPAVPPAFVYPSTDEMTKQLWEQAFGPQAKGWPDLFGKLADGSRGKYTVEAIADLHADAGL

SEQ ID NO: 146 - protein, 327 aa, synthetic construct
MSFINKQGYWISENGWRMCDTAELDYTAVPGTSFNLGVRKGAPNIILKAAIARLHREVEPMITSQIGCYTGTNSMPNSNHNSATAFDYNWNKHPYQEWGTWGANRAKVDRIVADFRGILEFGGNWTSPRDEMHFELHFAEGHAGTEQLATDLRNGLWGIWAPGQAPEPGAPSTPIPDGYLQIGSEGDQVRKLQVGMNKVFPNYRAMPLDEDGIFGPMTEAAVTEFQQRSLIGADGIVGPETKARLATYGIVLDGASAPTSPPAVPPAFVYPSTDEMTKQLWEQAFGPQAKGWPDLFGKLADGSRGKYTVEAIADLHADAGLHHHHHH

SEQ ID NO: 147 - protein, 579 aa, synthetic construct
MSFRNVYGNDWSENGWRMCNRDECVLVDGPFMNTAPLRRGPAEIILGDFVRRYHAQVAPVISPVWGWSLYNDVDDSNHLSATGVDINAPQWPWGEKRMPQWLIDRVNALLADYYIDGECGIYWGRNWNRPDEMHFQLNWPEGDPRYARLIAKIQGKPPVVVNPPNPGGSVGADGKAILRIGSKGAEVRQLQAGMNVVFEDYSHLDEDGEYGPMTAAVVAEFQRRSHLKITDYGVVGAITRKELNRYNILGAAKGTAPSVPAPPEPEGEAPRPIYIYSAAGTGAQWWQGPPFEAGQWCKGLNIHHQPVGYPAGGFLGLMGGDPKISYNDSITNLGDELERLIDSNPDIDNPDIEFWFFGYSQSADGMKRKVTELFGDGGKYSHLRSRINGLILFGDPTRAPGPTKVGNNPPGWGIARWDAPEWVDKLTWSITTHNDLYACTTDDTLVKYFYPWFIRAETELPFVIYSAKIIIPALLNLAAPFLGGALGGVATPILSAITGLAGSFIGQLLGGITGGDDPDPELIKLLSIQGILSNIPQLIKTLVALQGIQTHGEYHLPKPEFDNRTGIQVACDIVRDFRR

SEQ ID NO: 148 - protein, 585 aa, synthetic construct
MSFRNVYGNDWSENGWRMCNRDECVLVDGPFMNTAPLRRGPAEIILGDFVRRYHAQVAPVISPVWGWSLYNDVDDSNHLSATGVDINAPQWPWGEKRMPQWLIDRVNALLADYYIDGECGIYWGRNWNRPDEMHFQLNWPEGDPRYARLIAKIQGKPPVVVNPPNPGGSVGADGKAILRIGSKGAEVRQLQAGMNVVFEDYSHLDEDGEYGPMTAAVVAEFQRRSHLKITDYGVVGAITRKELNRYNILGAAKGTAPSVPAPPEPEGEAPRPIYIYSAAGTGAQWWQGPPFEAGQWCKGLNIHHQPVGYPAGGFLGLMGGDPKISYNDSITNLGDELERLIDSNPDIDNPDIEFWFFGYSQSADGMKRKVTELFGDGGKYSHLRSRINGLILFGDPTRAPGPTKVGNNPPGWGIARWDAPEWVDKLTWSITTHNDLYACTTDDTLVKYFYPWFIRAETELPFVIYSAKIIIPALLNLAAPFLGGALGGVATPILSAITGLAGSFIGQLLGGITGGDDPDPELIKLLSIQGILSNIPQLIKTLVALQGIQTHGEYHLPKPEFDNRTGIQVACDIVRDFRRHHHHHH

SEQ ID NO: 149 - protein, 501 aa, synthetic construct
MSFVWFRPEGPLRTREQIAREVHAVSLARGLDELASVLTLMCIDVEAGADDNNGERQWWCPWNANDPTSKNYPHDSQSDDGRSVGYLQQQNGAAGEVVSGSDNWWGPMRSRMTLALAVDVFQTRLADDYGRAAGNPKLAGEFVQRVQRSGYPDRYAQRWDEAWAVLRRALAQGPVTPKPPLPPIGTGSPITRSRLTSNRYVGRGGKTPRWIVVHTQEGGRSAWDLAGFLKSTEGQGVRSVSYNACVDDTETVLTVNWDDTPWSAVNANPYAFHICMAGSYSGWDRGKWLETDARDGKNEDLQLTRTAQLIAWLCRTYDIPADYIGGSGIPWGRDGICGHRDFGSWGGGHTDPGPDFPWDELIRRVRLYLDTNTGDQDMAQVPQSEWQEVIDYVRAQNTPIPSASPLRHLGEGNVNTRANLARAIDANQHVTAVVTLAKEGHTPSIALLWEVSTAADAPGKYPDRQEDAKLAKTLLASISKTKKAVAAEDIEAWLDAEKAAA

SEQ ID NO: 150 - protein, 507 aa, synthetic construct
MSFVWFRPEGPLRTREQIAREVHAVSLARGLDELASVLTLMCIDVEAGADDNNGERQWWCPWNANDPTSKNYPHDSQSDDGRSVGYLQQQNGAAGEVVSGSDNWWGPMRSRMTLALAVDVFQTRLADDYGRAAGNPKLAGEFVQRVQRSGYPDRYAQRWDEAWAVLRRALAQGPVTPKPPLPPIGTGSPITRSRLTSNRYVGRGGKTPRWIVVHTQEGGRSAWDLAGFLKSTEGQGVRSVSYNACVDDTETVLTVNWDDTPWSAVNANPYAFHICMAGSYSGWDRGKWLETDARDGKNEDLQLTRTAQLIAWLCRTYDIPADYIGGSGIPWGRDGICGHRDFGSWGGGHTDPGPDFPWDELIRRVRLYLDTNTGDQDMAQVPQSEWQEVIDYVRAQNTPIPSASPLRHLGEGNVNTRANLARAIDANQHVTAVVTLAKEGHTPSIALLWEVSTAADAPGKYPDRQEDAKLAKTLLASISKTKKAVAAEDIEAWLDAEKAAAHHHHHH

SEQ ID NO: 151 - protein, 480 aa, synthetic construct
MSFVWLADKPLRSREEIAQIVHAVSLARGLDELASAMVLMCIDVEVGANDRNGKRQWWCPWNAKDPSSQKFPHDSESNDGRSVGYFQQQNGRAGEVLPVDDRDNWWGSMTSRMTLALAADTFLDRLSDDYRGAADNPALAGQFVQRVQGSAFPDRYAQSWNEAWSVLRRALDGTTEPSEPGVLTPTPGFSGDPFWLADVLRAEGLNVVEMDGWRERGEGDQGVLWGAVFHHTGNVNETPEGIAFHPDLGLAAHLLIRPDGTVWVCGIGMANHAGVGSWPGIRTDNANPVTIGVEVAILPEKDAPHRTGWPAVQYDATVKTFAAILRKLALGSDRAISHKEWAQLGPAGYRQGKWDPGAIDMNIFRADVQAQIGSNTGGFLMALSDTEQREILSFVREMKTQVASLSPFRHLGEEAANNIPGFVRVIDANSHVEIVEKLAGYGDQGALALLHEIADADLERYPDRRNDAELARRVLAKVAK

SEQ ID NO: 152 - protein, 486 aa, synthetic construct
MSFVWLADKPLRSREEIAQIVHAVSLARGLDELASAMVLMCIDVEVGANDRNGKRQWWCPWNAKDPSSQKFPHDSESNDGRSVGYFQQQNGRAGEVLPVDDRDNWWGSMTSRMTLALAADTFLDRLSDDYRGAADNPALAGQFVQRVQGSAFPDRYAQSWNEAWSVLRRALDGTTEPSEPGVLTPTPGFSGDPFWLADVLRAEGLNVVEMDGWRERGEGDQGVLWGAVFHHTGNVNETPEGIAFHPDLGLAAHLLIRPDGTVWVCGIGMANHAGVGSWPGIRTDNANPVTIGVEVAILPEKDAPHRTGWPAVQYDATVKTFAAILRKLALGSDRAISHKEWAQLGPAGYRQGKWDPGAIDMNIFRADVQAQIGSNTGGFLMALSDTEQREILSFVREMKTQVASLSPFRHLGEEAANNIPGFVRVIDANSHVEIVEKLAGYGDQGALALLHEIADADLERYPDRRNDAELARRVLAKVAKHHHHHH

SEQ ID NO: 153 - protein, 529 aa, synthetic construct
MALGMILENGWPECDLVDCDYATIPGTPLRLPFQKGHPFIILQAFLRDLDQYIEPVMNARGITDEGSWTEDNSVYTSNHKGATAFDYNWDDHPMGRAGAGWDGSVLIAGDQVPAVQELLAWYEGMVFWGNNWSSPKDSMHFQMGYDTYGPANAARVQNFIDRKIRADGYSTWRRGGTARGGGVVPPVAVPVQTGLTANLLQSIGGYRKDMTLARYQALLPELIDAFHFADLNTIDRRAMGIAQLFHESGALRYQEEIADGSAYEGRTDLGNTQRGDGKRYKGRDFLQITGRSNYTALSAWAFARKIPGADSPTFFVDRPELLATDRFAFLGFAWYWTTRRNKAGQSLNDMADARNIDGATLMVNGGYNGLDSRKTFYARALAANADLLDPEPVDPLEELLMSDRKVPSASIYATPGEEDIPLVELLRAIDAALHRTAIVEPDAELGDPDAIDRMLRTAAGKGQYGTLPGPVNHAKAKLAKIAAANPPALLYVARAAKAGDVAALGVITDLQNTNPAVLQAFVAAQKGAN

SEQ ID NO: 154 - protein, 535 aa, synthetic construct
MALGMILENGWPECDLVDCDYATIPGTPLRLPFQKGHPFIILQAFLRDLDQYIEPVMNARGITDEGSWTEDNSVYTSNHKGATAFDYNWDDHPMGRAGAGWDGSVLIAGDQVPAVQELLAWYEGMVFWGNNWSSPKDSMHFQMGYDTYGPANAARVQNFIDRKIRADGYSTWRRGGTARGGGVVPPVAVPVQTGLTANLLQSIGGYRKDMTLARYQALLPELIDAFHFADLNTIDRRAMGIAQLFHESGALRYQEEIADGSAYEGRTDLGNTQRGDGKRYKGRDFLQITGRSNYTALSAWAFARKIPGADSPTFFVDRPELLATDRFAFLGFAWYWTTRRNKAGQSLNDMADARNIDGATLMVNGGYNGLDSRKTFYARALAANADLLDPEPVDPLEELLMSDRKVPSASIYATPGEEDIPLVELLRAIDAALHRTAIVEPDAELGDPDAIDRMLRTAAGKGQYGTLPGPVNHAKAKLAKIAAANPPALLYVARAAKAGDVAALGVITDLQNTNPAVLQAFVAAQKGANHHHHHH

SEQ ID NO: 155 - protein, 469 aa, synthetic construct
MVLRTENGWYQVPASSCEWTTIPGTNVTLQIQKGWPLTIMRAFAADFNAYVEPLRDPDSACWTPTNSVATSNHLSGTAMDLNWNSHPFQVLNAGFDAAKIATIRELLAFYEDTIFWGNDWTTPKDAMHFQMGYDTYNNSHTQDFINRKIRPDGYSTFRRGEAPSVDVAQVLSDAMGGRLSLDRYAQLLPAVTASLLACDCTNVNRIAMWCAQIGHESAGLYYTEEIASGAAYEGRADLGNTQPGDGVRFKGRSWIQITGRSNYTRLSAWAFSKGLIPSSTYFVDNPTALASDEYAGLGAAWYWVVARPDINALSDARDLVTVTKRINGGTNGLSDRQDRYNRALAMGDQLLALTTGGDDMFTDADRDLLKQIAEYRRKSLSPLRWPYEGAVNTCAGFAWTADANIHMVLVEKLAVEYGDPQSVALLLAVSRTDEPGREDDTALAAKILTKVDPDAVSAGERWLAEHRVD

SEQ ID NO: 156 - protein, 475 aa, synthetic construct
MVLRTENGWYQVPASSCEWTTIPGTNVTLQIQKGWPLTIMRAFAADFNAYVEPLRDPDSACWTPTNSVATSNHLSGTAMDLNWNSHPFQVLNAGFDAAKIATIRELLAFYEDTIFWGNDWTTPKDAMHFQMGYDTYNNSHTQDFINRKIRPDGYSTFRRGEAPSVDVAQVLSDAMGGRLSLDRYAQLLPAVTASLLACDCTNVNRIAMWCAQIGHESAGLYYTEEIASGAAYEGRADLGNTQPGDGVRFKGRSWIQITGRSNYTRLSAWAFSKGLIPSSTYFVDNPTALASDEYAGLGAAWYWVVARPDINALSDARDLVTVTKRINGGTNGLSDRQDRYNRALAMGDQLLALTTGGDDMFTDADRDLLKQIAEYRRKSLSPLRWPYEGAVNTCAGFAWTADANIHMVLVEKLAVEYGDPQSVALLLAVSRTDEPGREDDTALAAKILTKVDPDAVSAGERWLAEHRVDHHHHHH

SEQ ID NO: 157 - protein, 542 aa, synthetic construct
MTEKVLPYDRAVVPQEYGWSCGPAATQVVLNSRGIVVSETSLLAQIEAVENPGRGDDRDGTDYVGLIERVLDNIVPDASYTSVYLERDPATAAQKEALWTNLKRSIDGGFGVVMNWVAPPSNKPRGVKGSVSPRYSGGTTYHYVAAMGYDDNPAARAVWIADSGFQPQGYWISFDQCATLIPPKGYAFADVTPATGGPSTPTTPTPSADQVSVLAQVMSPSVVARDRFAALLPAVAQCLAECGCTTVERIAMWAAQIGTESGGLRYMEEIASGAEYEGRCSDLGNCQPGDGIRFKGRGPIQVTGRGHYTNLSQWAHGKGLVPTPTFFVDDPGQLASDRYGFIGVTWYWTTQRPMNDYADRRDIEGGSRAVNGTNPKTGRANGIDDRIGRYNRALAMGDALLTLTTTTGSGDITMSAAEELEAQSRGVFPPSDEQKRGGWPQPWRFVYSRFARPFNSIVKDAARGPWGRVQPDNTWQNGHTDSDEQTVTIGEQIAWRNEFSDGITRDHGDVMIELMEDLIARRKSAGQSTSPATDALKAAQKS

SEQ ID NO: 158 - protein, 548 aa, synthetic construct
MTEKVLPYDRAVVPQEYGWSCGPAATQVVLNSRGIVVSETSLLAQIEAVENPGRGDDRDGTDYVGLIERVLDNIVPDASYTSVYLERDPATAAQKEALWTNLKRSIDGGFGVVMNWVAPPSNKPRGVKGSVSPRYSGGTTYHYVAAMGYDDNPAARAVWIADSGFQPQGYWISFDQCATLIPPKGYAFADVTPATGGPSTPTTPTPSADQVSVLAQVMSPSVVARDRFAALLPAVAQCLAECGCTTVERIAMWAAQIGTESGGLRYMEEIASGAEYEGRCSDLGNCQPGDGIRFKGRGPIQVTGRGHYTNLSQWAHGKGLVPTPTFFVDDPGQLASDRYGFIGVTWYWTTQRPMNDYADRRDIEGGSRAVNGTNPKTGRANGIDDRIGRYNRALAMGDALLTLTTTTGSGDITMSAAEELEAQSRGVFPPSDEQKRGGWPQPWRFVYSRFARPFNSIVKDAARGPWGRVQPDNTWQNGHTDSDEQTVTIGEQIAWRNEFSDGITRDHGDVMIELMEDLIARRKSAGQSTSPATDALKAAQKSHHHHHH

SEQ ID NO: 159 - protein, 477 aa, synthetic construct
MSFTQFLQDDPLLAREQIMAIAIDVAKTLGMPDVKGAAILNVMAVSVEVGVEDNDPPHARRFWCPANHNDGDSFNYSHDSVSNDNRSIGYLQQQKGPNGELWWGTTAQQMDPHQAFISFQTRLKKRGYDASNAQSAGEAAQAIQGSAFPDRYREQWDDINALYDAVAGTITAPTTPTQPSFSFTEKNIIDGENASNRQGHNPRLFVLHTEEGNLLGQALDNWMDNNEVSYHYAVDPDASGAVAWDLVDTDLSSWSVLSANPDSINLVFAGSYAAMSRTDWLSKYGKAIKVAAYLAVQDCRKYGIETTVRVGFAAGGYSSLKTSNGITDHYGITKGLGIGTHTDVGPNFPWDVFASYVAQFANGTTEEDDMFTDADRALLTRVHFELTNKWNSRSIYAEPGEGPVDTLAGMVLNDDGMEHSELVERLAVLGDPDSLRRVIRVAAGEGVVTDAATVARAAKILAEVPADILTAYEAAAK

SEQ ID NO: 160 - protein, 483 aa, synthetic construct
MSFTQFLQDDPLLAREQIMAIAIDVAKTLGMPDVKGAAILNVMAVSVEVGVEDNDPPHARRFWCPANHNDGDSFNYSHDSVSNDNRSIGYLQQQKGPNGELWWGTTAQQMDPHQAFISFQTRLKKRGYDASNAQSAGEAAQAIQGSAFPDRYREQWDDINALYDAVAGTITAPTTPTQPSFSFTEKNIIDGENASNRQGHNPRLFVLHTEEGNLLGQALDNWMDNNEVSYHYAVDPDASGAVAWDLVDTDLSSWSVLSANPDSINLVFAGSYAAMSRTDWLSKYGKAIKVAAYLAVQDCRKYGIETTVRVGFAAGGYSSLKTSNGITDHYGITKGLGIGTHTDVGPNFPWDVFASYVAQFANGTTEEDDMFTDADRALLTRVHFELTNKWNSRSIYAEPGEGPVDTLAGMVLNDDGMEHSELVERLAVLGDPDSLRRVIRVAAGEGVVTDAATVARAAKILAEVPADILTAYEAAAKHHHHHH

SEQ ID NO: 161 - protein, 476 aa, synthetic construct
MGRRNVYGYDYSSNGWPMVDEGSCTWVTVPGTSVSLQIQNGQPLAILRAFAADFNAYVEPLRDPDSASWTPGNSVPTSNHLSGTAMDLNWDSHPFQIANAGFSAAQIATVKEIQAFYEGTVFWGNDWSDPKDAMHFQLASLANGGDINTYQNAHTADFIARKIRADGFSTFRRGNKPIGGVSILAAATGLPEARAAEILPAVSDGLKASQCTNVNRIAMWLAQVGHESGSFVYTEEIASGAAYEGRADLGNTQPGDGVRFKGRSWIQITGRNNYGLFSQWAFRQRLVQSPTYFVDHSTELADLKWAGIGAAWYWTVARTDINALSDRQDLETVTRRINGGTNGLADRRDRYNRALLQGDALLQLLAPAEPLDPIEELLMSGLAVPSMSIYADPDEPDVPVVDMIRALDAHGPHEPYVENQARLGDPDALRRVARTAAGRGKYGTASGPVNQAKSVLEGIRVTNPAVLEQFDAQSGA

SEQ ID NO: 162 - protein, 482 aa, synthetic construct
MGRRNVYGYDYSSNGWPMVDEGSCTWVTVPGTSVSLQIQNGQPLAILRAFAADFNAYVEPLRDPDSASWTPGNSVPTSNHLSGTAMDLNWDSHPFQIANAGFSAAQIATVKEIQAFYEGTVFWGNDWSDPKDAMHFQLASLANGGDINTYQNAHTADFIARKIRADGFSTFRRGNKPIGGVSILAAATGLPEARAAEILPAVSDGLKASQCTNVNRIAMWLAQVGHESGSFVYTEEIASGAAYEGRADLGNTQPGDGVRFKGRSWIQITGRNNYGLFSQWAFRQRLVQSPTYFVDHSTELADLKWAGIGAAWYWTVARTDINALSDRQDLETVTRRINGGTNGLADRRDRYNRALLQGDALLQLLAPAEPLDPIEELLMSGLAVPSMSIYADPDEPDVPVVDMIRALDAHGPHEPYVENQARLGDPDALRRVARTAAGRGKYGTASGPVNQAKSVLEGIRVTNPAVLEQFDAQSGAHHHHHH

SEQ ID NO: 163 - protein, 480 aa, synthetic construct
MANRIVYGLTHSSNGWPMVDEGSCTWVKIPGTSVTLQIQNGQPLAILRAFAADFNAYVEPLRDADSACWTPTNSVSSSNHLSGTAMDLNWGDHPFYSNYGGYTPAEIATVRELLDFYEGFMFWGQDWTNPKDCMHFQLSSLANGGPVNTYGNPAVDNFIARKIRADGFSTFRRGNAPVGGAPVLAAATGLSEARAAEILPAVQSGLRQSDATTVPRIAQWLAQVGHESVSFYYTEEIAKNGRYAPYIGRTWIQITWDYNYRAFSEWCFDRGLVPTRDYFVVNYQRLADLEWAGLGASWYWTEQRPMNALVDAGENASWNGYRGFSAVTAAINGGTNGLADRQARYNRALALGDRLLTLIDSDSGDDLASVPQDQWDRVFREQTQKLPSRSALRRLGEGVIDTWAGIDLNQDGNLHVLLVKTLAELGDTDSLDLLREVASADPVRYPDRQKDRQLALRILADIEANKPGVLQNYLAEKGIA

SEQ ID NO: 164 - protein, 486 aa, synthetic construct
MANRIVYGLTHSSNGWPMVDEGSCTWVKIPGTSVTLQIQNGQPLAILRAFAADFNAYVEPLRDADSACWTPTNSVSSSNHLSGTAMDLNWGDHPFYSNYGGYTPAEIATVRELLDFYEGFMFWGQDWTNPKDCMHFQLSSLANGGPVNTYGNPAVDNFIARKIRADGFSTFRRGNAPVGGAPVLAAATGLSEARAAEILPAVQSGLRQSDATTVPRIAQWLAQVGHESVSFYYTEEIAKNGRYAPYIGRTWIQITWDYNYRAFSEWCFDRGLVPTRDYFVVNYQRLADLEWAGLGASWYWTEQRPMNALVDAGENASWNGYRGFSAVTAAINGGTNGLADRQARYNRALALGDRLLTLIDSDSGDDLASVPQDQWDRVFREQTQKLPSRSALRRLGEGVIDTWAGIDLNQDGNLHVLLVKTLAELGDTDSLDLLREVASADPVRYPDRQKDRQLALRILADIEANKPGVLQNYLAEKGIAHHHHHH

SEQ ID NO: 165 - protein, 484 aa, synthetic construct
MKDDYARAILHAGRELGITPRGIVIAFATVYVESDWIMYANHKVPESLNLPHERVGSDGRSVGLFQQQVVWGNGAWWWGDAATCMDPHKSARLFFQRLARLDYNSDANSPGWYAQQVQRSAFPDRYDPRMSAAQALYNRILGTTSARPDESPEPLAGWQGDPVWLADVLREALGDRLVVEAGWQGRGNGAGPNRQMDDIWGVMIHHTGNDRETVAAIRDGVEQPGGFLAGPLAQCLIKPDGRCHLVAVGPCNHAGQGYYPGVGTNTGNIRLIGFECAWPTVNPDGSYNRGQPWPDAQIHTMRDAATAVVRRLGYGSDRVIGHKEYATRAPNVKWDPGNLSMDWFRLQVAKDLRGEFDPKPADQPRVEVPEPSPTGIPPDFTQQMFLQMSGRWEMLGWQTIVESLAEIRDATIGSSDAGKPGFTLGGRPQTPAAVLAARKTPGTNASATKASARKAPAEKAPAKKARARKVRAKKASAEKAAARG

SEQ ID NO: 166 - protein, 490 aa, synthetic construct
MKDDYARAILHAGRELGITPRGIVIAFATVYVESDWIMYANHKVPESLNLPHERVGSDGRSVGLFQQQVVWGNGAWWWGDAATCMDPHKSARLFFQRLARLDYNSDANSPGWYAQQVQRSAFPDRYDPRMSAAQALYNRILGTTSARPDESPEPLAGWQGDPVWLADVLREALGDRLVVEAGWQGRGNGAGPNRQMDDIWGVMIHHTGNDRETVAAIRDGVEQPGGFLAGPLAQCLIKPDGRCHLVAVGPCNHAGQGYYPGVGTNTGNIRLIGFECAWPTVNPDGSYNRGQPWPDAQIHTMRDAATAVVRRLGYGSDRVIGHKEYATRAPNVKWDPGNLSMDWFRLQVAKDLRGEFDPKPADQPRVEVPEPSPTGIPPDFTQQMFLQMSGRWEMLGWQTIVESLAEIRDATIGSSDAGKPGFTLGGRPQTPAAVLAARKTPGTNASATKASARKAPAEKAPAKKARARKVRAKKASAEKAAARGHHHHHH

SEQ ID NO: 167 - protein, 285 aa, synthetic construct
MPQAAGWRGDPVWLADVLRAEGLDVAEFAGWRGRGHGDFHDIRGVMVHHTGSDFATAASIAYGRPELPGPLSQVHIGRNGTVTVVAAGVAWHAGVGMYPWLPVNMGNWHTIGIECANSGTAPWAPHRQNWPEAQYFALVNTCAAISRRLGLPAARTIGHKEYAGRAQGKWDPGAIDMDRLRLDVARQIGAPARATSPRPAVPVGRYADVLLYRGTRGRQVAELQRRLKFAYASYAGGLEISGEFDVATEEAVREFQRRTPGLHVDGIVGPATAAALELRLIDVDL

SEQ ID NO: 168 - protein, 291 aa, synthetic construct
MPQAAGWRGDPVWLADVLRAEGLDVAEFAGWRGRGHGDFHDIRGVMVHHTGSDFATAASIAYGRPELPGPLSQVHIGRNGTVTVVAAGVAWHAGVGMYPWLPVNMGNWHTIGIECANSGTAPWAPHRQNWPEAQYFALVNTCAAISRRLGLPAARTIGHKEYAGRAQGKWDPGAIDMDRLRLDVARQIGAPARATSPRPAVPVGRYADVLLYRGTRGRQVAELQRRLKFAYASYAGGLEISGEFDVATEEAVREFQRRTPGLHVDGIVGPATAAALELRLIDVDLHHHHHH

SEQ ID NO: 169 - protein, 434 aa, synthetic construct
MTAAEFAPAVLQAGRDLGISPKGIVIGFATVYVESNWTMYANAKVPDSMNIPHEAVGSDGYSVGLFQQQVVNSGNGWWWGDAATCMDPYKSAQLFFSRLKRLDYNSGAQSPGSYAQAIQQSAFPDRYDQRMADAQALYDQISGGTVTTPPQDPRLTALQKVRPDFNELEFWCDNNQDRAGTKVDLWLIHTQEPGNLTDNDAALHLAQFLKSTEGSGNPVSYHYTIRQARDGGVTVYDIVDTDLASWSVGNSNNRAINACFAGSGVAWSRADWMTQAKAIDVAAYLAVQDCLKYGIDPNVIPGPQYGQNPPGISDHRYCSNWLKDGNNHDDVGDNFPWDVFSAAVAKYWAAANAGTTPPPADTGGDTTPADPFEAWVAGATDRQLLEYIVEQLGPGDPSWNSKGATLRDFLWSLQSQAKQTAAKAAGKRASRKSR

SEQ ID NO: 170 - protein, 440 aa, synthetic construct
MTAAEFAPAVLQAGRDLGISPKGIVIGFATVYVESNWTMYANAKVPDSMNIPHEAVGSDGYSVGLFQQQVVNSGNGWWWGDAATCMDPYKSAQLFFSRLKRLDYNSGAQSPGSYAQAIQQSAFPDRYDQRMADAQALYDQISGGTVTTPPQDPRLTALQKVRPDFNELEFWCDNNQDRAGTKVDLWLIHTQEPGNLTDNDAALHLAQFLKSTEGSGNPVSYHYTIRQARDGGVTVYDIVDTDLASWSVGNSNNRAINACFAGSGVAWSRADWMTQAKAIDVAAYLAVQDCLKYGIDPNVIPGPQYGQNPPGISDHRYCSNWLKDGNNHDDVGDNFPWDVFSAAVAKYWAAANAGTTPPPADTGGDTTPADPFEAWVAGATDRQLLEYIVEQLGPGDPSWNSKGATLRDFLWSLQSQAKQTAAKAAGKRASRKSRHHHHHH

SEQ ID NO: 171 - protein, 523 aa, synthetic construct
MSWVSFEPRPMRSREEIARIVHGVSLRRGLDELATAIALMTIAVESGSNGHWWCPWNAKDSTSRNYQFDSQSDDGFSVGYFQQQNRRPGETPSGSDDWWGPMRSRMTLEDAADQFLARLTHDWRSAADNPALAGRFAQDVQGSGFGERYQQHWDEAWDVLRRALAEAAVPEPTLVAKKSGEVMGFVGDPVWLEDVLREALGDRLVVEPDWEKRGTGGAMGDVWGVMIHHTGNINETVSAIRDGVHQPGGFLPGPLAQCLIKPDGTCHLIAVGPCNHAGRGSYQGVGTDCGNRRLIGFECCWPTPRPELPGGCDTCEPWSAPLIITMRDATAAVLTKLGYSSERVIGHKEYAGSAQGKWDPGNLSMDWFRGEVQKDLDDAVFPGEQPMPAPEPDPDPVLPPKLLPEPNPRSDRQLLEEIWDQLRGPGGNGWRQLGGNTVVDYLHEIGCFATDLRHQLDELSTMLEALTTRGLSTTPNGSTAPRKAAAKKVPPKKIPAKKTPAKKTAASAATRKPHAKKPAKKTS

SEQ ID NO: 172 - protein, 529 aa, synthetic construct
MSWVSFEPRPMRSREEIARIVHGVSLRRGLDELATAIALMTIAVESGSNGHWWCPWNAKDSTSRNYQFDSQSDDGFSVGYFQQQNRRPGETPSGSDDWWGPMRSRMTLEDAADQFLARLTHDWRSAADNPALAGRFAQDVQGSGFGERYQQHWDEAWDVLRRALAEAAVPEPTLVAKKSGEVMGFVGDPVWLEDVLREALGDRLVVEPDWEKRGTGGAMGDVWGVMIHHTGNINETVSAIRDGVHQPGGFLPGPLAQCLIKPDGTCHLIAVGPCNHAGRGSYQGVGTDCGNRRLIGFECCWPTPRPELPGGCDTCEPWSAPLIITMRDATAAVLTKLGYSSERVIGHKEYAGSAQGKWDPGNLSMDWFRGEVQKDLDDAVFPGEQPMPAPEPDPDPVLPPKLLPEPNPRSDRQLLEEIWDQLRGPGGNGWRQLGGNTVVDYLHEIGCFATDLRHQLDELSTMLEALTTRGLSTTPNGSTAPRKAAAKKVPPKKIPAKKTPAKKTAASAATRKPHAKKPAKKTSHHHHHH

SEQ ID NO: 173 - protein, 482 aa, synthetic construct
MADRTVYGNTHSENGWRMVDQGSCVWVKVPGTNVTLQIREGQPAAIMGAFAADYNAHVEPLRDADSACWTATNSVGTSNHLSGTGMDLNWNGPDGRTFRLGISKERAYPGDKARKLDELLTFYEGIIFCGGEWSIRDWMHMQMGGNTYGARNVERVNDFIRRKIRPDGFSTFKRGGATAPPALPAPPPAGGNQVDVLARATGISTAKATEILPLVVFALGKCNCTNPRRIAAALAQWVIESGHFVYTEEIASGPESQERWKYKGRTWIQLTWLENYLGFSRWVHSLGLVPTPTYFGDRPRELADLKWAALGPAYWWAIKYPQINEYADRGDIDNVSKWVNAPAWVDNPNKHANHEKERRDAYNRALALGDRLLTLIATPEPLDELEAIMATEIDSWSIYANPGEPKIPVVNLIAAIDAKIHRDLTDEDARAGDPEAIRRVARTAAGLGRVKTPGAIQHAVRVLNEIQATNPQYILAAIPGKA

SEQ ID NO: 174 - protein, 488 aa, synthetic construct
MADRTVYGNTHSENGWRMVDQGSCVWVKVPGTNVTLQIREGQPAAIMGAFAADYNAHVEPLRDADSACWTATNSVGTSNHLSGTGMDLNWNGPDGRTFRLGISKERAYPGDKARKLDELLTFYEGIIFCGGEWSIRDWMHMQMGGNTYGARNVERVNDFIRRKIRPDGFSTFKRGGATAPPALPAPPPAGGNQVDVLARATGISTAKATEILPLVVFALGKCNCTNPRRIAAALAQWVIESGHFVYTEEIASGPESQERWKYKGRTWIQLTWLENYLGFSRWVHSLGLVPTPTYFGDRPRELADLKWAALGPAYWWAIKYPQINEYADRGDIDNVSKWVNAPAWVDNPNKHANHEKERRDAYNRALALGDRLLTLIATPEPLDELEAIMATEIDSWSIYANPGEPKIPVVNLIAAIDAKIHRDLTDEDARAGDPEAIRRVARTAAGLGRVKTPGAIQHAVRVLNEIQATNPQYILAAIPGKAHHHHHH

SEQ ID NO: 175 - protein, 337 aa, synthetic construct
MAWTGNVLWLAEALRAEGLRVIEHPGWQQRGHGEYRDIRGVMCHHTAGGGPNDWQVVLNGRPDLPGPLSNLVLEKDGTYRVIAAGVCWHAGRGSWPGWPTDNANWHVIGIEAVSRGVPDAQGRYDWTQEQLDAYVRGCAVIAHRAGFSVRDVVGHKEYSSEGKIDPAGIDMDDFRAQVQQVLDRMRAPRDLDAFPLPPGYYYGPLEGPTQSISGLHPSERPEWREALKRWQRAQRVPETGVWDAETITAAKAVQSANGWNPDGLIGPGTWAAGLRGTVPPAEPEPAPVPLPEPEPVPEPAPVPEPEPAPGPEPVGASPRTVLREVVRWLRDRIPVGK

SEQ ID NO: 176 - protein, 343 aa, synthetic construct
MAWTGNVLWLAEALRAEGLRVIEHPGWQQRGHGEYRDIRGVMCHHTAGGGPNDWQVVLNGRPDLPGPLSNLVLEKDGTYRVIAAGVCWHAGRGSWPGWPTDNANWHVIGIEAVSRGVPDAQGRYDWTQEQLDAYVRGCAVIAHRAGFSVRDVVGHKEYSSEGKIDPAGIDMDDFRAQVQQVLDRMRAPRDLDAFPLPPGYYYGPLEGPTQSISGLHPSERPEWREALKRWQRAQRVPETGVWDAETITAAKAVQSANGWNPDGLIGPGTWAAGLRGTVPPAEPEPAPVPLPEPEPVPEPAPVPEPEPAPGPEPVGASPRTVLREVVRWLRDRIPVGKHHHHHH

SEQ ID NO: 177 - protein, 547 aa, synthetic construct
MSFTRFLQDDPLLTREQVMAELIRVADELNMPDKRGACVIAGMTISQEVGVKDNDPPFERRFWCPANRADPESFNYPHDSESNDGRSVGYFQQQKGPNGELWWGTTASEMNLHSAATQFMTRLKAAGYNASNAQAANDSAQAIQRSGVPQAYKQWWDDINRLYDKVKGSGGGPAPAPKPPQSGPWTGDPVWLADVLRAEGLNVVELPGWLDRGHGDMGRLWGVVCHHTGSDNTPSSEIAFHPSLGLCSQIHLARNGTVTLCGVGIAWHAGVGSYPGLPEDNANAVTIGIEAQNSGTYDGAPHRTNWPDAQYDAYVKCCAAICRRLGVRADHVISHKEWAGRKQGKWDPGAIDMNIFRADVQRRIDAHQPNGEDDFMAALSADEQREVLNLLRVLADRRFVSRSPFRHLGEGPSETVAGFGLNTDGLNHAQYTIELARLGDPTHLALLREVASAEGDSRYPDRQYDAKLAKRVLAEIEGAATAPAKPSTPSAPTEPAPEAPTPPVKAACALSAAGCVVAGSTSGGGCALSTDGTGKCVVTAATDGGAA

SEQ ID NO: 178 - protein, 553 aa, synthetic construct
MSFTRFLQDDPLLTREQVMAELIRVADELNMPDKRGACVIAGMTISQEVGVKDNDPPFERRFWCPANRADPESFNYPHDSESNDGRSVGYFQQQKGPNGELWWGTTASEMNLHSAATQFMTRLKAAGYNASNAQAANDSAQAIQRSGVPQAYKQWWDDINRLYDKVKGSGGGPAPAPKPPQSGPWTGDPVWLADVLRAEGLNVVELPGWLDRGHGDMGRLWGVVCHHTGSDNTPSSEIAFHPSLGLCSQIHLARNGTVTLCGVGIAWHAGVGSYPGLPEDNANAVTIGIEAQNSGTYDGAPHRTNWPDAQYDAYVKCCAAICRRLGVRADHVISHKEWAGRKQGKWDPGAIDMNIFRADVQRRIDAHQPNGEDDFMAALSADEQREVLNLLRVLADRRFVSRSPFRHLGEGPSETVAGFGLNTDGLNHAQYTIELARLGDPTHLALLREVASAEGDSRYPDRQYDAKLAKRVLAEIEGAATAPAKPSTPSAPTEPAPEAPTPPVKAACALSAAGCVVAGSTSGGGCALSTDGTGKCVVTAATDGGAAHHHHHH

SEQ ID NO: 179 - protein, 485 aa, synthetic construct
MLTVESFAAAMGNSLSVDRYRQLFPAAVESMVACGCTTVNRAAMWLAQVGHESGGLRWMEELASGAAYEWRSDLGNTQAGDGVRFKGRGPIQITGRYNYRKVSEWAHAQGIVPTPTYFVDNPTQLASDQYGFIGVSWYWQHGGPRPGQINGFADAGDILSGSRCVNGWVTTPNGMPDRTERWNRCRAMGDQILPAGAGTDNGGDGMAWTGDPVWLEDVLRPVLGDRLRVLPSWQMYGHGDFKDIRGVMVHHTGNARETAESIRKGRPDLRGPLSNIHIAPDGTVTLVAAGVCWHAGAGSYPWLPTNNANWHMIGIECAWPTIRPNGTYDEREPWPDAQIIAMRDTCAALTKRLGWDASRVIGHKEYAGASQGKWDPGNLDMGWFRGEVAKAMRGEFDPKAPAPAPNPPADNDNGGFLMALNDKQQHDLYNAVMTIAAVAADIQTQLRGPGLRGWPQLGRNANGEALTLVDAVGVIKAIADREAGR

SEQ ID NO: 180 - protein, 491 aa, synthetic construct
MLTVESFAAAMGNSLSVDRYRQLFPAAVESMVACGCTTVNRAAMWLAQVGHESGGLRWMEELASGAAYEWRSDLGNTQAGDGVRFKGRGPIQITGRYNYRKVSEWAHAQGIVPTPTYFVDNPTQLASDQYGFIGVSWYWQHGGPRPGQINGFADAGDILSGSRCVNGWVTTPNGMPDRTERWNRCRAMGDQILPAGAGTDNGGDGMAWTGDPVWLEDVLRPVLGDRLRVLPSWQMYGHGDFKDIRGVMVHHTGNARETAESIRKGRPDLRGPLSNIHIAPDGTVTLVAAGVCWHAGAGSYPWLPTNNANWHMIGIECAWPTIRPNGTYDEREPWPDAQIIAMRDTCAALTKRLGWDASRVIGHKEYAGASQGKWDPGNLDMGWFRGEVAKAMRGEFDPKAPAPAPNPPADNDNGGFLMALNDKQQHDLYNAVMTIAAVAADIQTQLRGPGLRGWPQLGRNANGEALTLVDAVGVIKAIADREAGRHHHHHH

SEQ ID NO: 181 - protein, 218 aa, synthetic construct
MSFRTVNGNTHTEDGWRCCNRDECDIVRIPELYLVDTAPLRKGAPLTILGAWLYWYDRNVEEITSPVWGWSATNDVLGTPGRNDGSNHLSGTAVDVMAPKYPWQQYTMNAATQAKVRKGLALFEGSVFWGRDWSRPDEMHYQMAWPEGDKRNDAFAAKLRAGYLGIYAPAQPPAPVQKRFPQDLSDRELLEYIAEQLGPGHPDWASKGMTLRDKVWSK

SEQ ID NO: 182 - protein, 224 aa, synthetic construct
MSFRTVNGNTHTEDGWRCCNRDECDIVRIPELYLVDTAPLRKGAPLTILGAWLYWYDRNVEEITSPVWGWSATNDVLGTPGRNDGSNHLSGTAVDVMAPKYPWQQYTMNAATQAKVRKGLALFEGSVFWGRDWSRPDEMHYQMAWPEGDKRNDAFAAKLRAGYLGIYAPAQPPAPVQKRFPQDLSDRELLEYIAEQLGPGHPDWASKGMTLRDKVWSKHHHHHH

SEQ ID NO: 183 - protein, 254 aa, synthetic construct
MSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQI

SEQ ID NO: 184 - protein, 260 aa, synthetic construct
MSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQIHHHHHH

SEQ ID NO: 185 - protein, 323 aa, synthetic construct
MWIGWQEGMSGRPVLAAKGELRAKFSYGKNLDLTEHFGPDLTEALKTFQRNKGGLRTDGVLDYATQKALGVLEALKPWVFTVAGTGAGWDAGYPAEVARAVLDLYRWQGVNYPAKPFPMGSSVDAGIAELKKLLRERMDRYPAARFVLIGYSQGAIVTSMVWQNFIKGSDLEARIIGSITYGNPCRELHVANGNVAEGIPVPEGRGISDFRLRSTPSWWYDFAHGANSRFGRDIYTDTPDDDAGEMMTAIYRLVQDLKNIIVGTDSLIEQVGEMFQRPITEVGAAMWAIFLGGQFVATRPYPTMPHINYDINPAVAILRRIAA

SEQ ID NO: 186 - protein, 407 aa, synthetic construct
MAWSPPSKVGDIAPVVAAAKQKIQKFQYGRDNVPSTDTSEVYTEGLLTAMLEFQRRVHNEVLAGQRETPDVPTDGTIDWATQVQLGIIERQKPTIPTVGPRHPAIVFRGTGGIIGQDLVSLVCQHCADLVEEINPDWPATMGGIPVGTAGNISDPSMWRGVQIAVADAQRRFLDILAKRPAVKIVIGGYSAGAVAAAIFKQWIMDNFPDHYLCSFSFGDPTRPVGGAFFAAPVIPWGRGVASVHYGDPKDWRHCWLTAEGDMYGQVPQGVTGDIMDDAFDMVTRVELSNILETAQAIIPRIPEVAEKAGIGLPSVLGALAGGIPGLLGLGLPWALGAISGLVGTGNPDLLTGSAAAAKAAQIALTFAAQGTAPHIEYHMREVWPGQTYLGLAIQHVRDYASATPATA

SEQ ID NO: 187 - protein, 285 aa, synthetic construct
MTTYGELKALRLGVKYVRHTLFTVAGTWADMWSGYPADVARLVDEDLFRWQPVWYPASFGPVGNPLGRSYQESVQDGVKELVRLINATPGTFALVGYSQGAEVVSRVLLEILFGSLRHRLKDFIGGGCFGNPYRAKGVSYPGSGLPTSGHGIAPVNLAPDILPAEMWEEWWNEGDLYAQNLDGKSGEIITSFYDILTKLQFHDMLGLAVNMFKALSNDKGIIAQVMRVLAVPLPGVIDAGRAVVYAGTFAVQGTRPHITLAETGRVARAVWHLNRIGAKTLARAS

SEQ ID NO: 188 - protein, 346 aa, synthetic construct
MRIGGEYVGLGLGDASEEIGRIRDFMRRKFRSYAGNLPDTRNALGLPLFDEPMTVAVTEMQGRYLQSGELRDGLFIFGIINAETKYVMGYLERPPGPDTRPLFITVCGTGVPWWVGPDADTARACEDKLFWQPIGYPAKNFPMGKSIAAGIDQCHVQFNRADPGFMHRQRIERNGVVLGGYSQGAVVVSELWENNIKPANGSLHWAKDYVVKAVTWGNPNREVGAVWPDYGGSPMASLTSQGVSSTGMRDTPSWWRNYAHAGDLYAAAEPGDSQQDKNAIWQIIRDLNFFTGTDSLLAQAIELSQMPIPRTIAAFKALIDAGMFFAKGTGPHVDYGIGPAIEYLRS

SEQ ID NO: 189 - protein, 285 aa, synthetic construct
MTTYGELKALRLGVKYVRHTLFTVAGTWADMWSGYPADVARLVDEDLFRWQPVWYPASFGPVGNPLGRSYQESVQDGVKELIRLINATPGTFALVGYSQGAEVVSRVLLEILFGSLRHRLKDFIGGGCFGNPYRAKGVSYPGSGLPTSGHGIAPVNLAPDILPAEMWEEWWNEGDLYAQNLDGKSGEIITSFYDILTKLQFHDMLGLAVNMFKALSNDKGIIAQVMRVLAVPLPGVIDAGRAAVYAGTFAVQGTRPHITLAETGRVARAVWHLNRIGAKTLARAS

SEQ ID NO: 190 - protein, 334 aa, synthetic construct
MPLRLGDRNESVRQWRIKMNAWFGPLYTRLHGPLPMDTDEFGPRAKSWQEEYERRTNQPVDGEVSDNDLRALNVPVPTKVVIFTVAGTGARWDQTYPYDLGRWQDQNRVILQPIGYPAATFPMGPSVDKGEAELVNQMRRHLDANPSLNFILVGYSQGAIVTSRVLRRMMSGDLAHYYNRCIAGVTFGNPMRERGHFVGVNDPGGQGLDPKPLVDTPSWWYDYAARGDIYSSGPGNNDRQAAEHMTSIYLAVMGKFILGNDALVSQVIELFTNPFAEVPAVVKAIASGIGFVTSNPPTAAHIEYHIRECVPGVTYFDHAMGYVRQVISANKRIA

SEQ ID NO: 191 - protein, 365 aa, synthetic construct
MATWDSILDYLRGDPCKVGYFGVPGTWSPWDAGYQADVGRLINSAWFYWQGVGYVAAFGPVNGPITNPSYAESVQEGVDETVRLILARPGPIVLGGYSQGAEVVYWVAREFLTGGRLAHRRGDLLLIVTFGNPCRAKGHGVNQATAHGWGISRKPPLTELLPIWLDYALPGDMYCCADDDTYLAIGYAALTKLQLHDPWQLVQAMLALIQSDEFADALAELLDPLFPGLATTLGELTGMDGAALLANRKPVGGGVLGGLMSGQLLTATPAGGNLLGGLIGAGTGMLGGLLDGVIPGGLPPIVGGLLGGGSGPGGTPTGMYKLGKTFAALLNFATTNDHGHYHDTPAFAGTNAVAHAVGEVNRLAA

SEQ ID NO: 192 - protein, 308 aa, synthetic construct
MSDRIFFLWLPGTWEVDSLKAENPSATNFVGLGKWLVDDRFPDRLPLNIFEPIMLCPPDYMASFGPIPAAGSSIFSAWNHLSYKESVLDAVEHSVDVILKLPTDRPIIIGGYSQGAEVAERLKAEFLPGGRLSSYFLLAHYTFGNPGRPQGVTFPNGNTLPWGGISNLNIPTPQGTFYRSYAFYDDMYANANPKSYLFEFYDGLTDLQFHDPFKAVKDVVGVVTKSDLMILAGAQPTNPFWVVTHIPQFIDISTKAVNSLDALARFASSGAHGHYGDWEIIPGFTPIFHCIRSAKYAAKGIGYAVPGI

SEQ ID NO: 193 - protein, 254 aa, synthetic construct
MSKPWLFTVHGTGQPDPLGPGLPADTARDVLDIYRWQPIGNYPAAAFPMWPSVEKGVAELILQIELKLDADPYADFALAGYSQGAIVVGQVLKHHIINPRGRLHRFLHRLRKVIFWGNPMRQKGFAHTDEWIHQVAASDTMGILEDRLENLEQYGFEVRDYAHDGDMYASIKEDDMHEYEVAIGRIVMSARRFIGGKDSVIAQLIELGQRPIWEGIAMARAIIDALTFFAKSTQGPSWPHLYNRFPAVEFLRRI

SEQ ID NO: 194 - protein, 341 aa, synthetic construct
MKGLDGSYIGIGLGDVSSEVLKIKQFINRKFSRFRLLETEIYDAAMEAAVRELQTIYKNNGTLTDPFIPGVVNLATKYAIGYLKKDVILPIHFSVEGHMSDMWIGPAAYVGEVLRAENRALHFPTGYDNRALPFNNKSGVEQLVQRVGATEFFIDGKVIKFPPGTPWTASAFSQGAMIWCDFYRQYLMPGKPLHWRLKDLRAVICCGNPDREKGVCVDWIPDKPDPDRQGIMDDENRMVNTPWYWLELARKGDMYTDNESSGERGLNKTAIAKIITQNKWSGGPAGLLARVTDLLVNPIDDVIPITLALYDAIRFGAGGIRAHGGYDMEPAVQFCRERLAA

SEQ ID NO: 195 - protein, 405 aa, synthetic construct
MAYEAPRKVGDTHKLIPAAKAKLAGNSYGKAIGADRSPTYTAAFGAALVQYGKNVHDAVAKGKRRTPDVNTAGVFDWAIQDQMELETKPAAPEAQPLPKGRAIGYVWRGTGGIVGQDLVYQVLQQCSDLVELVDPPWAATMGGIPVGTAGGVGDPSMWSAVQETVAWTQADFIRRRAADPKVRVVLGGYSAGAIACAIFKAWLLENYPENYVCSYSFGDPTRPFGGGFFGMPAPWGRGISTLSYGDPKDYRHCWLTHEDDMYAQIPGGVVGDIMDDVYEEVTRFAFRDIMQVAMRMMTALPKIAEKAGISLPAVFASLAAGPAGLITFALPMLLSSLGGLIPGSTPDDQLTGTPAAAKAATIGLKFLFAGTRPHTVYHLDPAWPGGPTFVQLAVMHVRDWASRPA

SEQ ID NO: 196 - protein, 288 aa, synthetic construct
MSLWPEIRAMLNGEFYVPAGTIISANGTGVPDGAGPGFGADLARELERVAEGMWIWRWIDYPAAVFPMRPSINVLRENLKAMIRATPGKLVLTAYSQSAVAFAYVWRDDILNPNGELHDRLDDIEAVILYGNPVRAPGIAYGNELGGQPAPGKLNGHVTGGIAGPDCLKPEECLHPVTGKRVVLDFANAGDLYAAAPVGETPWVEETEVGHNETMIYEAVMDFNGKDILAFAKEIAQILVLPLTQVVPLVQAIINGLSFLVKGPKAPHWTYDIRPAVDHLVRRGNELR

SEQ ID NO: 197 - protein, 584 aa, synthetic construct
MSLDNHHIEELEQALRDVKRIGVQTNPLLAPSPAHVTGPTWQRTKDGKFYLPEKTLGYQILYWMSQYLLIPGGPDAGQPFKPTREQGRFILWWYAVDEFGRFVYRNGLLRRLKGWGKDPLAGALALAELCGPVAFSHFDTDGNPIGKRKPAAWIQVAAVSQDQTRNTFSLFPVLASDRLKEEFKLDFNKTIVYAKTIGGVIEAATSNPLTLEGKRPTFVIKNETQWWVETNNGHSMSEVIAGNVDKAAYGGCRSLAICNGHIPGQDSDAERDYDAHLDVLAGKAIDTGFLYDALEAPADTPLSEIPSPIEDPEGFEEGIAKLREGLIIARGDAVWLDLDTIIASMLDKRRPVTESRRKFLNQINAHEDSWIAPHEWNACHPEEGVPPLAKGDRITLGFDGSKSNDHTALVACRVEDGCLFLIKTWDPEKYPGGEVPRDEVDATVRSMKAGYDVVAFRADVKEFEAYVDQWGKDFRKTVQVNASPGNPIAFDMRGNQKKFALDCERFLDAVLERELFHDDNPILRQHVFNARRHPTNYDAIAIRKASKDSSKKIDAAVCAVLAFGARQDFLMSKKNRTRRAAVIM

SEQ ID NO: 198 - protein, 402 aa, synthetic construct
MATVDYRLGMSGDQVKVIKRLLIDGYYYVRERYPRMTAASDVYDVYTQASIVEFQFRAGLPVTGVADYATQVRLGAVVPPPPPRQRIMVLTFSGTSADMWTGYPADVARALDPSIFYWQPVCYGPNGIPAIFPMGSSAKSGEVEGLRLLDEKAGDFDYIVLIGYSQGALPASRLMRRILSGDLQRFKAKLIAGVTFGNPMREKGHTFPGGADPGGHGLDPQCLVNTPDWWHDYAAKGDIYTVGSGGNDETANADMTFIYQLVQGDILAMLFGTGNPLDILGALGGGLLGGFGGGGLLGGNKGGLQLPSGLVLPGVGLGQGGALTQRQRGLVEAVLALLANPFAEVPPAVKAIVSGIGFVATNPPTAPHIEYHIREAAPGVTYFQHAIDYLRQVGASVAARAA

SEQ ID NO: 199 - protein, 285 aa, synthetic construct
MSEPMLLTAQGTGVDMYTGYPHDLGLRLRDEGLVELQPIGKYPARMFPMGPSVKIGVDEGVDLVLKAEARPSREVPDGYLLSGYSQGAWLVSDLLDEFRGGRLKHLRHKLAGGATFGNPRRGLDENGGRGIADKLIVDTPDTWVDEFDPGDIYANVPNNDIGEDMTAIFKLVRLNGISDVIDLRSALGLGGVLGGIMPGGNQLASMLGGLLGGGEQNNITEQIVEMLKSPLREFPAAVQAIVKAAVFFGRKPATAPHIEYHIRTVTPGGPTYFEHAVSHMRAVAA

SEQ ID NO: 200 - protein, 406 aa, synthetic construct
MAYQAPRKVGDTHPLIPAAKRKLAGNSYGKAIGDDRSNVYTAAFGAALVQYGKNVHDAVLRGKRRSPDVNTVGVFDWAVQDQMELEEKPAAPVAQPLPKDRAIGYVWRGTGGIVGQDIVYEVIRQCSDLVELVDPPWAATMGGIPVGTAGGIGDPSMWQAVQETVAWTQADFIARRNANPKIRVVLGGYSAGAIACAIFKAWLLENFPENYVCSYSFGDPTRPKGGGFFGMPAPWGRGISTLSYGDPNDYRHCWLTHEEDMYAQIPGGVAGDIMDDVYEEVTRFAFRDIMQVAMRMMTALPKIAEKAGISLPAVFASLAAGPVGLITFALPLLMGSIGSLINQDQDKDSLTGTPAAAAAAMIGLKFLFAGTRPHIVYHLDPAWPGGPTFVQLAVMHVRDWASRPAA

SEQ ID NO: 201 - protein, 325 aa, synthetic construct
MVWIGWQEGMDGLPVLAAKGELRAKFSYGKNLDTTTHFGPDLTAALQTFQRNKGGLRTDGVLDYATQKALGVLEALKPWAFTVAGTGAGWDAGYPADVARAVLDLYRWQGVNYPAAPFPMGDSVNAGIAELIKLLKQRLDRYPAAKFVLIGYSQGAIVTSMVWKRYIKGTPLEDRIIGSITYGNPCRELGVANGNVAQGIPVPEGRGIADDLLESTPSWWYDFAHGANSPFGRDIYTDTPDDDAGEMMTAIYRLVQSLKNILFGTDSLLEQVGELFKRPLAEVYAAFRAIVYGGQFFITRPYPTMPHITYDINPAIQILRRIAKN

SEQ ID NO: 202 - protein, 407 aa, synthetic construct
MAWAPPSKVGDIAPVVAEAKKKIQKFQYGRDNVPSTDTSEVYTEGLMTAMLEFQRRVHDEVLAGKRETPDVPTDGTIDWATQVQLGIIERQKPTIPTVGPRHPAIVFRGTGGIIGQDLVSLVCQHCADLVEEINPDWPATMGGIPVGTAGNISDPSMWRGVQIAVADAQRRFLDILAKRPAVKIVIGGYSAGAVAAAIFKQWIMDNFPDHYLCSFSFGDPTRPVGGAFFAAPVIPWGRGVASVHYGDPKDWRHCWLTAEGDMYGQVPQGVTGDIMDDAFDMVTRVELSNILETAQAIIPRIPEVAEKAGIGLPSVLGALAGGIPGLLGLGLPWALGAISGLVGTGNPDLLTGSAAAAKAAQIALTFAAQGTAPHIEYHMREVWPGQTYLGLAIQHVRDYASATPATA

SEQ ID NO: 203 - protein, 308 aa, synthetic construct
MTDRIFFLWLPGTWEVDMFKNENPSATNFVGLGKWLVDDRFPDRLPLSIFEPIMLCPPDYMASFGPIPAAGSSIFSAWNHLSYKESVMDAVEHSVQIILKLPVSRPIVIAGYSQGAEVAERIKAEFLPGGRLERYFLLSHYTFGNPGRPQGVTFPNGNILPWGGISNLNIPTPQNTYYRSYAFYDDMYANANPKSYLFQFYDGLTDLQFHDPFKAVKDVLGVVTKSDLMILAGAQPTNPLWIMMNIPKFIDIGTRAANSLDALARFASSGAHGHYHDWEIIPGFTPIFHCIRSAKYAAKGIGYAVPGI

SEQ ID NO: 204 - protein, 326 aa, synthetic construct
MAWIGWQEGMEGLPVLAAIGELRAKFSYGKQLELTQKFTPELTAAVQTFQVKKGGLRTDGVLDYDTQKALGVLEALKPWVFTVAGTGAFWDAGYPADIARAVADLFRWQGVNYPAKPFPMGDSVDAGIAELLKLLKQRLDRYPAAKFALIGYSQGAIVTSMVWKRYIKGNEDLEKRIIGSITFGNPCRELGVANGNVAEGIPIPEGRGIGDDRLQSTPEWWYDFAHGANSPFGRDIYTDTPDDDTGEMMTAVYRAVQKLANLWSGVDSILEQVGEIIKRPIPEIYAAFRAVIYGGQFVTTQPWATYPHTNYNIDTAVAFLRRIAKN

SEQ ID NO: 205 - protein, 290 aa, synthetic construct
MSKPMLLTGQGTGADMFTGYPHDLGVRLRDEDRVELQPIGKYPASMFPMGPSVKIGVDEGVDLVLRAEARPAREVPDGYLLCGYSQGAWLVSDLLDEFRGGRLKHVAHKLAGGVTFGNPRRELDQSGGRGIADTLIVGTPDTWVDEFDPGDIYANVPNNDVGEDMTAIFKLVRLNGISDVVDLRSGLDLAGVLGGLLVPGVGQLGGLAGGLGALFGGAQEQNTIVEQVVEMLKSPLREFPAAVMAIVKAAVFFGRKPATAPHIEYHIRTVTPGGPTYFEHAVNHMRAMAA

SEQ ID NO: 206 - protein, 583 aa, synthetic construct
MELTEQDIENLEKAVAEAKRIEPVTDPELAPSPPHIIGPTWQRRADGRFYLPEKTLGYQILMWMSEYLLIPGGPKAGEPFKPTREQGRFILWWYAVDEFGRFVYRNGLLRRLKGWGKDPLAGALALAELCGPVMFSHFDSEGNPVGKRKPSAWIQVAAVSQDQTRNTFSLFPVLASDRLKEEFKLDFNKTIVYAKAIGGVIEAATSNPLTLEGKRPTFVIKNETQWWIETNNGHAMSEVIAGNVDKSAYGVCRSLAICNGHIPGQDSDAERDYDAYMDVLAGKAIDTGFLYDALEAPADTPLSEIPPPTEDPEGFEKGIAQLREGLKIARGDAVWLDLDTIIASMLDKRRPVTESRRKFLNQINAHEDSWIAPHEWDRLALTEDLFKLKKGDRIALGFDGSKANDHTALVACRIEDGMLFLIKHWNPEHHGGEVPRDDVDATVRSCFERYDVVAFRADVKEFEAYVDQWGKDFRKKVQVNASAGNPVAFDMRGQQKRFALDCERFLDAVLEREVYHDGNPVLRQHVLNARRHPTNYDAIAIRKASKDSSKKIDAAVCAVLAFGGRQDFLMSKKNRSRRAAVIM

SEQ ID NO: 207 - protein, 403 aa, synthetic construct
MAWVGWQLGMQGEQVKVIQQKLIGKYQWVRDRYPRLTASGVYDVFTQAAIVEFQFRVGLPVTGIADYATQVRLGAVVPAPPPRQRIMVLTFSGTSADMWTGYPADVARALDPSIFYWQPVCYGPNGIPAIFPMGSSAKSGEVEGLRLLDEKARDFDYIVLIGYSQGALPASRLMRRILSGDLQRFKSKLIAGVTFGNPMREKGHTFPGGADPGGHGLDPQCLVNTPDWWHDYAAKGDIYTVGSGSNDEKANADMTFIYQLVQGDILGMMFGTGNPLDILGLLGGLGGGLLGGLGGGLLGGGKGGLQLPSGLVLPGVGLGQGGALTDHQRGLVEAVLALLANPFAEVPAAVKAIVSGVGFIATNPPTAPHIEYHIREAAPGVTYFQHAIDYLRQVGASVAARAA

SEQ ID NO: 208 - protein, 365 aa, synthetic construct
MATWDSILDYLRGDPCKVGYFGVPGTWSPWDAGYQADVGRLINSAWFYWQGVGYVAAFGPVNGPITNPSYAESVQEGVDETVRLILARPGPIVLGGYSQGAEVVYWVAREFLTGGRLAHRRGDLLLIVTFGNPCRAKGHGVNQATAHGWGISRKPPLTELLPIWLDYALPGDMYCCADDDTYLAIGYAALTKLQLHDPWQLVQAMLALIQSDEFADALAELLDPLFPGLATTLGELTGMDGAALLANRKPVGGGVLGGLMSGQLLTTTPAGGNLLGGLIGAGTGVLGGLLDGVIPGGLPPIVGGLLGGGSGPGGTPTGMYKLGKTFAALLNFATTNDHGHYHDTPAFAGTNAVAHAVGEVNRLAA

SEQ ID NO: 209 - protein, 341 aa, synthetic construct
MKGLDGSYIGIGLGDVSSEVLKIKQFINRKFSRFKLLETEIYDAAMEAAVRELQTIYKNNGTLTDPFIPGVVNLATKYAIGYLKKDVILPIHFSVEGHMSDMWIGPAAYVGEVLRAENRALHFPTGYDNRALPFNNKSGVEQLVQRVGATEFFIDGKVIKFPPGTPWSASAFSQGAMIWCDFYRQYLMPGKPLHWRLKDLRAVICCGNPDREKGVCVDWIPDKPDPDRQGIMDDENRMVNTPWYWLELARKGDMYTDNESSGERGLNKTAIAKIITQNKWSGGPAGLLARVTDLLVNPIDDVIPITLALYDAIRFGAGGIRAHGGYDMEPAVQFCRERLAT

SEQ ID NO: 210 - protein, 346 aa, synthetic construct
MRIGGEYVGLGLGDASEEIGRIRDFMRRKFRSYAGNLPDTRNALGLPLFDEPMTVAVTEMQGRYLQSGELRDGLFIFGIINAETKYVMGYLERPPGPDTRPLFITVCGTGVPWWVGPDADTARACEDKLFWQPIGYPAKNFPMGKSITAGIDQCHVQFNRADPGFMHRQRIERNGVVLGGYSQGAVVVSELWENNIKPASGSLHWAKDYVVKAVTWGNPNREAGAVWPDYGGSPMASLTSQGVSSTGMRDTPSWWRNYAHAGDLYAAAEPGDSQQDKNAIWQIIRDLNFFTGTDSLLAQAIELSQMPVPRTIAAFKALIDAGMFFAKGTGPHVDYGIGPAIEYLRS

SEQ ID NO: 211 - protein, 584 aa, synthetic construct
MSLDNHHIEELEQALRDVKRIGVQANPLLATSPAHVTGPTWQRTKDGKFYLPERTLGYQILYWMSQYLLIPGGPDAGQPFKPTREQGRFILWWYAVDEFGRFIYRNGLLRRLKGWGKDPLAGALALAELCGPVAFSHFDSDGNPIGKRKPAAWIQVAAVSQDQTRNTFSLFPVLASERLKEEFKLDFNKTIVYAKTIGGVIEAATSNPLTLEGKRPTFVIKNETQWWVETNNGHSMSEVIAGNVDKAAYGGCRSLAICNGHIPGQDSDAERDYDAHLDVLAGKAIDTGFLYDALEAPADTPLSEIPSPIEDPEGFEEGIAKLREGLIIARGDAVWLDLDTIIASMLDKRRPVTESRRKFLNQINAHEDSWIAPHEWNACHPEEGVPPLAKGDRITLGFDGSKSNDHTALVACRVEDGCLFLIKTWDPEKYPGGEVPRDEVDATVRSMKAGYDVVAFRADVKEFEAYVDQWGKDFRKTVQVNASPGNPIAFDMRGNQKKFALDCERFLDAVLERELFHDDNPILRQHVFNARRHPTNYDAIAIRKASKDSSKKIDAAVCAVLAFGARQDFLMSKKNRTRRAAVIM

SEQ ID NO: 212 - protein, 334 aa, synthetic construct
MPLRLGDRNESVRQWRIKMNAWFGPLYTRLIGPLPMDTDEFGPRAKAWQEEYERRTNQVVDGEVSDNDLRALGVPVPTKVVIFTVAGTGARWDQGYPFDLGRWQDQSRVILQPIGYRAAVFPMGPSVNEGEVELIRQMHIHLDRDPSLNFILIGYSQGAIVTSRVLRRMMSGDLAHYYDRCIAGVTFGNPMRERGHFVGSNDPGGQGLDPKPLVNTPTWWYDYAAKGDIYSSGPGNDDKQAAEHMTAIYQAVMGHFIIGNDSLTQQVIELFTNPFKEAPAVIKAIASGIGFVASNPPTAPHIEYHLRECVPGVTYFDHAMGYVRQVISANKRIA

SEQ ID NO: 213 - protein, 325 aa, synthetic construct
MAWIGWQEGMEGLPVLAAIGELRAKFSYGKQLELTQKFTPELTQAIKTFQVKKGGLRTDGVLDYATQAALGVPEALKPWVFTVAGTGAGWDAGYPADVARAVLDLFRWWGIVYPAKPFPMGDSVDVGIAELLKQMKLKLDRYPAAKFVLIGYSQGAIVTSMVWKRYIKGKDLEKRIIGSITFGNPCRELGVANGNVAEGIPIPEGRGIGDDRLQSTPEWWYDFAHGANSPFGRDIYTDTPDDDTGEMMTAVYRAVQKLKNLWSGVDSILEQVGEIIKRPIPEIYAAFRAVIYGGQFVTTQPWATYPHTNYIIDTAVAFLRRIAKN

SEQ ID NO: 214 - protein, 290 aa, synthetic construct
MSLWPEIRAMLNGEFYVPAGTIITANGTGVPDGAGPGFGADLARELERVAEGMWIWKWINYPAAVMPMRPSINTLRLALKEMIRTTPGKIVLSAYSQSAVAFAYVWRDDILNPDGELHDRLDDIVAVILYGNPVRAPGIAYGNELGGQSAPGKLNGHVTGGIAGPDCLRPEECVHPVTGRRIVLDFANAGDLYAAAPVGAEPWVKETEVGHNETLIYEAVMDFNGRDILAFAKEIAQILTMPLSQVVPLVQAIINGLSFLVQGPKAPHWTYTIGPAVDYLVRTGNELRKN

SEQ ID NO: 215 - protein, 400 aa, synthetic construct
MATVDYRLGMSGDQVKVIKRLLIDGYYYVRERYPRMTAASDVYDVYTQASVVEFQFRAGLPVTGVADYATQVRLGAVVPPPPPRQRIMVLTFSGTSADMWTGYPADVARALDPAIFYWQPVCYGPNGIPAIFPMGSSAKSGEAEGLRLLDEKAGDFDYVVLIGYSQGALPASRLMRRILSGDLQRFKSKLIAGVTFGNPMREKGHTFPGGADPGGHGLDPQCLVGTPDWWHDHATKGDIYTCGSGGNDETANADMTFIYQLVQGDILGMLFGTGNPLDLLGALGGGLLGGLGGGLLGGKGGLQLPSGLVLPGVGLGQGGALTQHQRGLVEAVLALLTNPFAEVPPAVKAIVSGVGFIATNPPTAPHIEYHIREAAPGVTHLQHAIDYLRQVGASVAARAA

SEQ ID NO: 216 - protein, 263 aa, synthetic construct
MPHPLNPTNKPVLLTLSGTGQNMWTGYAIDVAHRVEDVWFVQPVGYGPGGVPAVWPMGRSAKSGVDEGVRLVLQEHSDAPGYSISGYSQGGGAAAMLLDEFRHGRLTSFGDRLLGGVTFGNPFREKGSYAGAVDPGGRGIADTRTVDTPAGWADYVDPGDIYANVPDNAVGDDMTLIYRLVWLDDVGDVLALVAKLMRLLVSPLREFPSVVEALVRGIMFYGSKPRCAAHVEYHLRECPGTGMTYVEHAARHLRQLGQQAAAA

SEQ ID NO: 217 - protein, 400 aa, synthetic construct
MAWVGWQLGMQGEQVKVIQQKLIAKYQWVRDRYPRLTASGVYDVNTQAAIVEFQFRAGLPVTGIADYATQVRLGAVAPAPPPRQRIMVLTFSGTSADMWTGYPADVARALDPSIFYWQPVCYGPNGIPAIFPMGSSAKSGEVEGLRLLDEKARDFDYIVLIGYSQGALPASRLMRRILSGDLQRFKSKLIAGVTFGNPMREKGHTFPGGADPGGHGLDPQCLVNTPDWWHDYAAKGDIYTVGSGSNDEKANADMTFIYQLVQGDILGMMFGTGNPLDILGLLGGLGGGLLGGLGGGLLGGGKGGLQLPSGLVLPGVQGGALTDHQRGLVEAVLALLANPFAEVPAAVKAIVSGVGFIATNPPTAPHIEYHIREAAPGVTYFQHAIDYLRQVGASVAARAA

SEQ ID NO: 218 - protein, 290 aa, synthetic construct
MSLWPEIRAMLNGEFYVPAGTIITANGTGVPDGAGPGFGADLARELERVAEGMWVWKWINYPAAVFPMRPSINALHLALKEMIRATPGNLVLSAYSQSAVAFAYVWRDDILNPDGELHDRLDDIVAVILYGNPVRAPGIAYGNELGGQSAPGKLNGHVTGGIAGPDCLRPEECVHPVTGRRIVLDFANAGDLYAAAPVGAEPWVKETEVGHNETLIYEAVMDFNGRDIMAFAKEIAQILTMPLSQVIPLVQAIINGLTFLAQGPRAPHWTYSIAPAVDYLIRCGNELRKN

SEQ ID NO: 219 - protein, 323 aa, synthetic construct
MWIGWQEGMSGLPVLAAKGELRAKFSYGKGLDLTEEFGPDLTEALKTFQRNKGGLRTDGVLDYATQKALGVLEALKPWVFTVAGTGAGWDAGYPADVARQVLDLYRWQGVSYPAQAFPMGRSVDAGVAELQRLLRERLDRYPAARFVLIGYSQGAIVTSMVWEKFIKGSDLEGRIIGSITFGNPCRELNVANGNVAEGVSVPEGRGISDFRLRSTPSWWYDFAHGANSRFGRDIYTDTPDDDTGEMMTAIYRLVQDLKNIFVGTDSLVEQVGEMFRRPIPEVGAAMWAIFLGGQFVATRPYPTMPHITYDINPAVAILRRIAA

SEQ ID NO: 220 - protein, 263 aa, synthetic construct
MPHPLNPTNKPVLLTLAGTGQDMWTGYAIDVAHRVEDVWFVQPVNYGPGGVPAVWPMGASAKTGVDEGVRLVLQEHSDAPGYAISGYSQGGGAASMLLDEFRHGRLASFGDRLRGGVTFGNPFREKGSYAGTVDPGGRGIADTRIVDTPAGWADYVDPDDIYANVPDNKVGDDMTLIYRLVWLDDAGDVLALVVKLLQLLTSPLREFPAVVEAIVRGIMFYGTKPPCAAHGEYHLRECPGTGMTHFEHAVQHLRQLGEQTAAA

SEQ ID NO: 221 - protein, 325 aa, synthetic construct
MAWIGWQEGMEGLPVLAAIGELRAKFSYGKQLGLTKKFTPALTEAVKTFQRNKGGLRTDGVLDYETQKALGVLEALKPWLFTVAGTGAFWDAGYPADIARAVADLYRWQGVNYPATAFPMGDSVDAGIAELVKLIKQRLDRYPAAKFVMIGYSQGAIVTSMVWKRYIKGTSLEERIIGSITYGNPCRELGVANGNVAEGIPVPEGRGIADDRLQSTPLWWYDFAHGANSPFGRDIYTDTPDDDAGEMMTAVYRAVQKLKNLWSGVDSILEQVGEIIKRPIPEIYAAFRAIVYGGQFVTTQPWATYPHTNYTIDHAVAILRRIAKN

SEQ ID NO: 222 - protein, 290 aa, synthetic construct
MSKPMLLTGQGTGADMFTGYPHDLGVRLRDEDRVELQPIGKYPASMFPMGPSVKIGVDVGVDLVLRAEARPAREVPDGYLVCGYSQGAWLVSDLLDEFRGGRLKHVAHKLAGGVTFGNPRRELDQSGGRGIADKLIVGTPDTWVDEFDPGDIYANVPNNDVGEDMTAIFKLVRLNGISDVVDLRSGLDLVGVLGGLLVPGVGQLGGLAGGLGALFGGAQEQNTIVEQVVEMLKSPLREFPAAVMAIVKAAVFFGRKPATAPHIEYHIRTVTPGGPTYFEHAVNHMRAMAA

SEQ ID NO: 223 - protein, 254 aa, synthetic construct; identical to SEQ ID NO: 183

SEQ ID NO: 224 - protein, 341 aa, synthetic construct
MKGPDGSYIGIGLGDVSSEVLKIKQFINRKFSRFKLLETEIYDAAMEAAVRELQTIYKNNGTLTDPFIPGVVNLATKYAIGYLKKDVILPIHFSVEGHMSDMWIGPAAYVGEVLRAENRALHFPTGYDNRALPFNNKSGVEQLVQRVGAAEFFIDGKVIKFPPGTPWTASAFSQGAMIWCDFYRQYLMPGKPLHWRLKDLRAVICCGNPDREKGVCVDWIPDKPGPDRQGIMDDENRMVNTPWYWLELARKGDMYTDNESSGERGLNKTAIAKIITQNKWSGGPAGLLARVTDLLVNPIDDVIPITLALYDAIRFGAGGIRAHGGYDMEPAVQFCRERLAA

SEQ ID NO: 225 - protein, 390 aa, synthetic construct
MELKIGSNDQNTNGEVTPWQEWFKRYASSYAPPVDGYFGTPDEAAVRMLQARLGLVIDGVFGDRTAERVGYRWKNNQGGPVAQRRKIWIYTAPGSGANWDQGPSFQLGERCKNVLHINHQPLYYQKGGYLGFMGGDPKYSYNEVIWDQYLSLKYCMDTNPDWQDALAMRKQNPQAVVEVEGWFSGYSQSADGMEEAVERLFGDGGPYELLRDRINGIIQFGNPSTRNTGIARKVRPEWIYKLVTNLNYLNDFYAVAPDPIRPAFYKVIVEAEMELPFFVHVLQIAVPVILNIIPVFGGFFGPLGQLGVAAAAGIKDMNALGSLMGMAGTNKDQDVHQDLIEMLSLTGLIRNIPGLISVVSALPGLQAHGGYEFDPVMMNAAYDVVAAFRR

SEQ ID NO: 226 - protein, 389 aa, synthetic construct
MAWVGWQLDMEGDQVKVIQTKLYAKYDWVKNRYPSLKAASGKYDKATQAAVVEFQFRTGLPVTGIADFATQLRLGSAVSAPPPKPKPRILLLTFSGTSADMWSGFPADLARALDPAIYYWQPVNYGPNGIPAVFPMGSSVKSGEVEGVRLLDEKADEYDFIVLVGYSQGAIVASRLKRRIQSGDLQRFKSKLIAGVTFGNPLREAGHTFPGGTDPGGYGLDPELLTNTEDWWHDYAAPGDIYTCASGTSDQRTNSDMTLIYELVQGDILKLIFGSDSSPLDILLTLSSGLLGGFKLPAAILLPGLGEGPAGALSTRQRGLVEAVIALFTNPFAEVPAMVKAIVSGIGFVAQSPPTAPHIEYHIREAVPGVTYLQHAINHLNFVGAGIKR

SEQ ID NO: 227 - protein, 741 aa, synthetic construct
MSYGLPTGTNINYGQPGFPDWVYQLGAAFNLRASTYPGHQESDRVEAGYARNPNRQNRGIDWAGAVPDMDRFAEYLLSTRGSLEQVIWQNPATGARIGVAGGKDVTQTAYYAADYSGHTDHVHTRQSEAIPMPDAPPKDTLFADVSEWQVPVDDSYPYPVLSIRVSDGSYQDRNFARNYTWMRAALNSGKLTFGIVYTYVRPQTWQSNAATVKQMIDAAGGLHPRVALMLDIESGGNPPGDQSGGINALYSALADYTGDPARIIGYGNVSDLNGMWRTKPPGIRLIVAGYGRLPTYPGMVAHQYTDGQGYGGGLPEGCPPFGNCDMNAANGLTPAEFAAACGISGDLQPEPDPEPGPPRPDPSPSDPPTTNSPCAGHASATKPSSKPSPRSATPYWAPTTGSEAGNAHLAHRHSRLSQRAALMVRHLALVYRGTGGIIGEDYVSRVCQPLADLVQEENPPWAATMGGLPVAAAGAPSDPSMNKGAADALTASIPMIERAISENPQRRIIIGGYSAGAYVAALVRRYVQQRHPDNYLCSFSLGDPTRPPGGAYYPFDASVQPGGQGIGSWHYGDVTDPRHCWLSNHTAPPNLGPDMYAITPLGVTGEIMQAAYDMVTDFSFSDILTATRAIVQAVPKIAEDMGIDVPDVLAALAGGIPGLAGYGIPLLVGALSGLIGFGNNDTLTGTAAGAAAARIGLTFLAAGTGPHIRYEVDEVWPGQTYLGLAIQHVRYWASTVQPDAA

SEQ ID NO: 228 - protein, 325 aa, synthetic construct
MKIGGQWVGYGLGDTGETVAAMKDFLRRKFSYAAGLAPDDIYDDAMVAVVVEMQARYGLPASGIMNYATQLRCGFVKPRQPTHERGTLFTCQGTGVDMWTGPPADTARAVTDLYFWQPVGNWPAAPFPMGPSIDAGRAELNVQIDAHPGDISMAGYSQGAMVVALTYMRDILPADGRLHHRLPDVKRVVTWGNPCRQQGKANGNKQAGWPIPEGHGVNDWLLTETPDSWLDYAHGANSPWGRDFYTDVQGDWGEDCTAICKFVMGQNIFAGPDSLLAQVIELVQRPIPETIAMFQAIINAGMFFAAQTGPHLNYDIGPAIAYLRS

SEQ ID NO: 229 - protein, 287 aa, synthetic construct
MSADGSKPVLLTASGTGADMWTGYPADVARRMEDLWYFQPINYRAAVFPMGASVDEGVNEGVRIVNEEIPVGTPTSLVGYSQGGSVVSRLLDEFRSGRLKHRQSDLVAGLTFGNPDRELGSYAGTKDPGGRGISNSRIKNTPPWWCDLAEPGDIYTNVPNNDVGEDMTAIFRFVQLRGIDDLIGEDSLLEQFIEIITGDSAIGLPKLDNLLRIPGMLAKIGVTGPLSGFPAAVMAIIKGIAFFGAKPATAPHIEYHIREIEPGVTYFDWGVRYLRAAGEHARARMSA

SEQ ID NO: 230 - protein, 281 aa, synthetic construct
MTGNEGSGRIWAYTVSGTWAAWNEGFPADVARALDPNAFRWQPVAYPASFGPVPPASSPTLPSYAESVHEGVAELIRLINLNAGPFVLIGYSQGAEVTSRVLLEIQHGQLAHRQQDLLGGVTFGNPCRQSGHTWPGDTLSGHGIAALRIANTPSQWNDYAHGGDLYACVPDGQAGDNCTAVYQAVTQLQIHDPVQLIEEMLTAFSGKGGLGEQLWELLTNPFNLTSVFEAIVIATRFATTQPPTLDHINYDADDVMDGSGRSSLRHAIDHLNALGRQAVAA

SEQ ID NO: 231 - protein, 291 aa, synthetic construct
MSRPLFISVNGTGVPDPYGPGFSGDIGRALTDPWNNVMASFWGPELANVFDWQPIGYDAAVMGMDKSARGAVYRKPGTYSSDDTGGIVAQVLARPKGTKHVLSGYSQGAIATGICLVECYFDPKGPLNDRLSDLKGVVNFGDPKRSPGIANGNKVAGLPMPKKLDGYTTGGIAGPGCLKPDQTPDWLLSCALDGDLYAAAPVGDDPWHNEPLVGQIETRIYDFIQSGKLSTGIMAIAKGIAQEFEHPLANTVALVQAIVNGLTFAAQGVQAPHWLYGPFVPAMVEWILAQV

SEQ ID NO: 232 - protein, 361 aa, synthetic construct
MAWRGYELGMTDPKDGDGNIVPGGMIWQIQDKLKRKYASYTGAVVVSGRYDGATFAAVGEFQNRAGLIGTGVKPDEVGIANYATLVRMGVVTTTPPRAPLTIFTAAGTWSDMWTGLQADVARALDRRYFFWQPIWYPASFGPVGGGPAPSYEESVALGVEEGIRLIKATPGQFALCGYSQGAEVVSRILIELVSGRLKDRLKDCLWFVAFGNPARQPGVCVGRDPGGSGISGIRLLVPESVTVLDYAIDGDMYCTTPDGTEGGTNMRAVYKALTKMQIHDPGRDIISALTGDPSLMRQLIKLFSDPVKGGIGLVDALFRLAKFAITGAHGRYGQYEVFPGVTPVRHAIETLNADAARLLAA

SEQ ID NO: 233 - protein, 357 aa, synthetic construct
MIVSGQFVGYGRGDTGPEVERVRYYLTEKFAWARAMGITHGDVFDELTEKVLIRFQIIVGLPATGIANYATRLRLYGLKPTPWQRITVYTFAGTWSASDWGIQADVARGLDASFFTWVPVEYPASFGPIPGGPSAGGGAPSYQESVAAAVSTGIRQVNNTPGKLMIGGYSQGGEAASLLLKEILGGSLTHRRGDLVGGYTFGNPSREANHTWPGDSLPGRGISPNRIVGTPSTWHDYAHAGDMYTATPNDDAGDDITAVYSVLTRLQIHDPWELAQSIVAALKGKGGLVEQITELLGNPLHLIDAARAAGIALQFLASGLRDHGNYWEDDVMDGSGRTSVQHAIDSMNAYGRKALAA

SEQ ID NO: 234 - protein, 455 aa, synthetic construct
MRRRHRDHGALPLERQQGRDARPATPGLISAGRNSSTALLVLVVGPTASIGFVTPARADTPMIRLGFRGRSVLMAWRGFELTDPPMSGPDIALIRGKLVAKFEWARTMGVTEGDVYDRITADAVAEFQRRVGLPENGIADFKTRVRLGSWPPPPPPRHAALTFRGTGGIVGVDYTSRVAHAAGLEEIPILYPGSMGGIPVGAESNPNAPSGNDSVEVAVELAVDWIERHPSRTFCLLGYSQGAIAASKVRAELLPGGRLERFAGNYFCGMMVANPSRAFGHTFFLGPVPDGEGISNFHVPREACTWDWCELVQPDDFYANVPLGDVGDVCRQGQNIVMDTTVSDPIGMMQKVIPHLIKMLDEAGVDLPPSPLGILNGVWAGLVSSLLPGVVPAQFGAETAAAVHAARLALTFFAAQPPTKPHITYEFVEVLPGRTYLQLGIDHVRDWSGRTPVRT

SEQ ID NO: 235 - protein, 381 aa, synthetic construct
MGFKMGSSGPEVGRWQEFMRRKFASYSEELPVDEHYGYFDGVVVNEAKRRLGLPQDGVADDDFLIRIGFSAGQARPLSTTWVYSAAGTRAPWSIGPPFEIGEHAKRRGLRHQPVDYPAGGFFGPPDPTMSFNESIKVLRNEWARLLRLNTAGDIVTISYSQGADGMQRATAELFGDGGEFASQRHRLRRAIMVGNPTRPSGPTKIGNDPRGAGIARWHPPDWLQAITFDIVTHYDMYACAEDTTLVPLFYPWFTRAETELSFVAYSAQVIVPTVASFYNIFIPPILGGLFGPLGVQALALVTRIPVDTLNELFKGISGEDPDPELVEALSAKGLLSDIPGLIKSLVALGGIRTHNEYHLPRPEFGNRTGIDVGIGLIDEIL

SEQ ID NO: 236 - protein, 300 aa, synthetic construct
MVQEQPIRTEKSAADPMPRHAVLTFAGTFEAPGTGFPSDVVKQANPDLVYEVPVVAPYSFGPIPPDSPTAPSYAESVEIAVTYATTWITAHPRQTFAIGGYSQGAEAASRVLASIMGGPLQWARPNLIGGYTFGNPARGLGHTFPGGTDPGGRGISSFNLTTAQIPKNTRGIDTWWDFANPGDLYTTTPDDQAGADLTAVYDEAVQLSIQRNLILNLIAGLTERGGLLQQAIGLVTNPLAGGPALAEAITLAVRFYDTSPPGLAHLDYNTVDALPGQSSVAVAVNHLNSIAAATPTHSAA

SEQ ID NO: 237 - protein, 331 aa, synthetic construct
MTGWADLAANIAGTKLVRHAVLTYNGTWGAGLVQYPSDVVNGLAQYVDDGLCEEVPCPYPASFGPIGGNAAAPSYQQSIQDALGWSADWFDANPNRTFVLDGYSQGAEAASRVYLELVAGTALEENFVGGITFGNPCKMAGAAAPGVSSLGPQWRGISSVNMTSLPAINGQVVWADYAHNTANGDAGNDMYPQVPNTAVGTIMTDVYTTATQAQLNNPTAFLQSMVTDLMQLVEDSGLLTGLKGGAAGLLAMGAGAAIGFLVDLIGGVNINATGAQADVAAAVLGLQFLAAPGGPTGPHISYLGELPGYSNLVADAVNFLHTIATLTPARA

SEQ ID NO: 238 - protein, 266 aa, synthetic construct
MLAGAIAVAGSALVTAGTAAAQPACPDLHWIGAAGSGERGAEVTMNDGMGRVVFNSMQDLHQLVQRDGRTMTAEAVNYPATPVPADDSILGWAGFINSVDAGTAALNAQYAAFVQRCPSSKVVLAGYSQGAMVVHRNLQAFSQSPNLAAALLVADGDRLPTDATYNLGSVTSVPGAGKGVAQDWPILAHAPAPLPLDVAARTVSVCDLGDAVCDYDPEAEDAMNPTAVAIHTSYARSAAGGYRWTAPLYQLIGAAPTANQVSAAAG

SEQ ID NO: 239 - protein, 235 aa, synthetic construct
MIELLFVDGTWSRPGARSPVGEALRKALDPSKVKFTYVDYPADFGPATGVGDVSYADSVMAGVAALSLAVERSQFDVVVAGYSQGAAVAVHYALRVLPRKPKHIVLALATVGDPHQPVHNGRSGIAGAITLSLRSFRRFVPGDPIADLDLGSPVRSAADLSRWMSVRSPEAARAWAYRTAAELPTRAQRWWEPWNWAAIGRAGEAIRNYLGTAHSTDYISQGHVRRLARDIESVA

SEQ ID NO: 240 - protein, 233 aa, synthetic construct
MIELLWVDGTWAPRGGSPASEALRRALDPRKVKFTYVNYPADFGPATGMGDLSYEESKSIGAAALDRAVEASPHLVVVGGYSQGAGVAVKYARDILPKRPRHQVLAVATLGDPHTPVHHGRSGIAGALHVPRPRFTEWASGDPIADLPLGSPLRIVADLTGWMSVRTPEAARAWAFKTAERLGRAQPWWNPFRWHDFARAGEDIRNYLGTAHSTDYDSGGHARRLARRIESVA

SEQ ID NO: 241 - protein, 340 aa, synthetic construct
MAWDGWKEGMAGPPVLAAKRELKRKFSYAKHLVENTFFDHDLTVALMTYQVAKNIELAKRGEPLLRTDGVLDWYTQKVLGLLDRKVVIFTVSGTGAVWSQGYPFDVAMRQDQSKVIVQPIGYPAAVFPMEHSANEGERELVAQMRRHLDANPSYVFILIGYSQGAMVVSRVLRRMMSGDLRQYFDRCIAGVTFGNPLRERGHFTGASDPGGQGLDPECLVDTPSWWHDYAIPGDIYTCGPGNYDLAALEHMRAIYLAVQGHFLTGRDNLGEQVLEVLMNPFAEVPAVVKAIVSGLGFVTANPPTAPHIEYHIRECFPGVTHFEHAVDYVRRAVSAGMRIE

SEQ ID NO: 242 - protein, 1528 aa, synthetic construct
MSAHRTLLLRLSDSGEPVTSCSYGQGVLTLPSLPLPQGKKLGDMPVYTVKLAIPAGSPVTRDGLIWTNCPPDFSTQFDREKFYKKIIKTSFHEDDHIDLDIYVPGTYCFYLSFKNDKDELETTRKFYFVVLPILSVNDKFIPLNSIAMQSVVSKWMGPTIKDWEKVFARVASKKYNMIHFTPLQHRGESNSPYSIYDQLEFDPTVFKSEKEVADMVERLRTEHNILSLTDIVFNHTANNSQWLLDHPEAGYNHKTSPHLISAIELDKKLLDFSEQMEALGYPVDLKTVDDLIKVMDGIKEHVIGELKLWEFYVVDVKQTVSELREKWGNSKSWSDDNIPSKDDSTNLAQFVRDNATEPGFGSLGERGSNKINIDKFAAILKKLHSEDYNNGIEELATKILNDINLPFYKEYDDDINEVLEQLFNRIKYLRIDDHGPKQGPITKKLPLSEPYFTRFKAKDGEEYALANNGWIWDGNPLVDFASSQSKAYLRREVIVWGDCVKLRYGKGPSDSPYLWERMSKYVEMNARIFNGFRIDNCHSTPLHVGQYFLDVARRVNPNLYVVAELFSGSEAMDCLFVERLGISSLIREAMQAWSEEELSRLVHRHGGRPIGSYKFVPLDDFPYPADVKIDEEYCAYNPDDHSVKCVSEIMIPKTLTATPPHALFMDCTHDNETPNQKRTVEDTLPNAALVAFCSSAIGSVYGYDEVFPQLLDLVQEKRTYSCAENTGISKVKTLLNNMREEIASEAVDIEDSEMHVHHDGQYITFHRTNAKNGKGWYLVARTKFHSSGDQMLPRIKLSQTKATFKAAFSLERTGDAPISDEIIEGIPTKLRELTGFDIGFDENTKETSILLPQDFPQGSIVIFETQQLGIDDSLDHFIRSGAIKATEKLSLESINYVLYRAEQEEYDYSEGRSGAYDIPDYGKPVYCGLQGWVSILRKIIFYNDLAHPLSNNLRNGHWAVDYVVNRLDLYKDKEGVAEVQEWLRSRMERIKQLPSYLVPSFFALVVGIMYGCCRLRAMQLMSDNVGKSTVFVQSLAMTSIQMVSAMKSTSILPDQNIAAMAAGLPHFSTNYMRCWGRDVFISLRGLLLTTGRYEEAKEHILAFAKTLKHGLIPNLLDAGRNPRYNARDAAWFFVQAIQDYVTIVPGGVSLLQEKVTRRFPLDDEYIPYDDPKAFSYSSTIEEIIYEILNRHAGGIKYREANAGPNLDRVMKDEGFNVEVNVDWETGLIHGGSQFNCGTWMDKMGESEKANSVGVPGTPRDGAAVEINGLLKSCLRFVLQLSKDGKFKYTEVTKPDGSKISLSSWNDLLQENFERCFYVPKNKEDDNKFEIDATIINRRGIYKDLYRSGKPYEDYQFRPNFTIAMVVAPELFTPDYAAGAIELADQVLRGPVGMRTLDPSDYNYRPYYNNGEDSDDFATSKGRNYHQGPEWVWCYGYFIRAYHYFNFLTNPKCQVEGSAKKLKPSSYLYRKLYSRLLKHREWIENSPWAGLAELTNKDGEVCNDSSPTQAWSTGCLLDLFYDLWISYEE

SEQ ID NO: 243 - protein, 1538 aa, synthetic construct
MSAHRTLLLRLSDSGEPVTSCSYGQGVLTLPSLPLPQGKKLGDMPVYTVKLAIPAGSPVTRDGLIWTNCPPDFSTQFDREKFYKKIIKTSFHEDDHIDLDIYVPGTYCFYLSFKNDKDELETTRKFYFVVLPILSVNDKFIPLNSIAMQSVVSKWMGPTIKDWEKVFARVASKKYNMIHFTPLQHRGESNSPYSIYDQLEFDPTVFKSEKEVADMVERLRTEHNILSLTDIVFNHTANNSQWLLDHPEAGYNHKTSPHLISAIELDKKLLDFSEQMEALGYPVDLKTVDDLIKVMDGIKEHVIGELKLWEFYVVDVKQTVSELREKWGNSKSWSDDNIPSKDDSTNLAQFVRDNATEPGFGSLGERGSNKINIDKFAAILKKLHSEDYNNGIEELATKILNDINLPFYKEYDDDINEVLEQLFNRIKYLRIDDHGPKQGPITKKLPLSEPYFTRFKAKDGEEYALANNGWIWDGNPLVDFASSQSKAYLRREVIVWGDCVKLRYGKGPSDSPYLWERMSKYVEMNARIFNGFRIDNCHSTPLHVGQYFLDVARRVNPNLYVVAELFSGSEAMDCLFVERLGISSLIREAMQAWSEEELSRLVHRHGGRPIGSYKFVPLDDFPYPADVKIDEEYCAYNPDDHSVKCVSEIMIPKTLTATPPHALFMDCTHDNETPNQKRTVEDTLPNAALVAFCSSAIGSVYGYDEVFPQLLDLVQEKRTYSCAENTGISKVKTLLNNMREEIASEAVDIEDSEMHVHHDGQYITFHRTNAKNGKGWYLVARTKFHSSGDQMLPRIKLSQTKATFKAAFSLERTGDAPISDEIIEGIPTKLRELTGFDIGFDENTKETSILLPQDFPQGSIVIFETQQLGIDDSLDHFIRSGAIKATEKLSLESINYVLYRAEQEEYDYSEGRSGAYDIPDYGKPVYCGLQGWVSILRKIIFYNDLAHPLSNNLRNGHWAVDYVVNRLDLYKDKEGVAEVQEWLRSRMERIKQLPSYLVPSFFALVVGIMYGCCRLRAMQLMSDNVGKSTVFVQSLAMTSIQMVSAMKSTSILPDQNIAAMAAGLPHFSTNYMRCWGRDVFISLRGLLLTTGRYEEAKEHILAFAKTLKHGLIPNLLDAGRNPRYNARDAAWFFVQAIQDYVTIVPGGVSLLQEKVTRRFPLDDEYIPYDDPKAFSYSSTIEEIIYEILNRHAGGIKYREANAGPNLDRVMKDEGFNVEVNVDWETGLIHGGSQFNCGTWMDKMGESEKANSVGVPGTPRDGAAVEINGLLKSCLRFVLQLSKDGKFKYTEVTKPDGSKISLSSWNDLLQENFERCFYVPKNKEDDNKFEIDATIINRRGIYKDLYRSGKPYEDYQFRPNFTIAMVVAPELFTPDYAAGAIELADQVLRGPVGMRTLDPSDYNYRPYYNNGEDSDDFATSKGRNYHQGPEWVWCYGYFIRAYHYFNFLTNPKCQVEGSAKKLKPSSYLYRKLYSRLLKHREWIENSPWAGLAELTNKDGEVCNDSSPTQAWSTGCLLDLFYDLWISYEEHHHHHHHHHH

SEQ ID NO: 244 - protein, 753 aa, synthetic construct
MPPFRTSRGRPLPLGPSLTPDGTNFALLCRHGRTVTLVILPAEGGSTPLAELPLDARTNRTGDHWHIRVHDLPEAFCYGWRVDGPHGPRTRFDPSRLLLDPSAVILSHGAEWAGTCETDPQRTSRRSMYRRGTRYNWDDDCPPLVDYEDTVIYEVHVRGFTCHPSSGVAAPGTFRGLVEKIPYLKWLGVTAVELMPVFEWDECDCPFVNPATGEKLTNFWGYNPIAFAAPKAAFAASAKQLGQTNEFRDMVKAMHAAGIEVILDVVFNHTGEGDDRGRTFSFRGLDNELYYLLDDSGRYLNFSGCGNTVNCNHPVVRDLIMTCLRYWVEDMHVDGFRFDLASILGRDRRGNVMVEPPVIESITEDGVMADTKLIAEPWDAGGLYQVGQFPFGRRWSEWNGQYRDDVRRFWKGDPGLTGAMASRVCGSSDLYQWNGRLPRHSVNFVTAHDGFTLYDLVSYNEKHNEANGEGNRDGSNDNHSWNCGAEGPTTDPAVLALRRRQARNLMTTLMISQGVPMLLAGDEFLRTQQGNNNAWCQDNDVSWVNWTLAEGNKDFVRFVRELIHLRKRHPALRRRRFFAGEFRSGDAPRPAPARAAEPHVIDVFPPAGPVRPGDAGLHPASDAATGLARSGRGADAPVPALADIHWHGVEPFRPDWGYNARVLAFALDGRFTGREGDPDYQIDEDFYVVFNAWSEALRFRIPASPTRRRWRRLIDTALPAPGDFVAEGEGPVVADGGTYTVAGFSTLVLISEP

SEQ ID NO: 245 - protein, 759 aa, synthetic construct
MPPFRTSRGRPLPLGPSLTPDGTNFALLCRHGRTVTLVILPAEGGSTPLAELPLDARTNRTGDHWHIRVHDLPEAFCYGWRVDGPHGPRTRFDPSRLLLDPSAVILSHGAEWAGTCETDPQRTSRRSMYRRGTRYNWDDDCPPLVDYEDTVIYEVHVRGFTCHPSSGVAAPGTFRGLVEKIPYLKWLGVTAVELMPVFEWDECDCPFVNPATGEKLTNFWGYNPIAFAAPKAAFAASAKQLGQTNEFRDMVKAMHAAGIEVILDVVFNHTGEGDDRGRTFSFRGLDNELYYLLDDSGRYLNFSGCGNTVNCNHPVVRDLIMTCLRYWVEDMHVDGFRFDLASILGRDRRGNVMVEPPVIESITEDGVMADTKLIAEPWDAGGLYQVGQFPFGRRWSEWNGQYRDDVRRFWKGDPGLTGAMASRVCGSSDLYQWNGRLPRHSVNFVTAHDGFTLYDLVSYNEKHNEANGEGNRDGSNDNHSWNCGAEGPTTDPAVLALRRRQARNLMTTLMISQGVPMLLAGDEFLRTQQGNNNAWCQDNDVSWVNWTLAEGNKDFVRFVRELIHLRKRHPALRRRRFFAGEFRSGDAPRPAPARAAEPHVIDVFPPAGPVRPGDAGLHPASDAATGLARSGRGADAPVPALADIHWHGVEPFRPDWGYNARVLAFALDGRFTGREGDPDYQIDEDFYVVFNAWSEALRFRIPASPTRRRWRRLIDTALPAPGDFVAEGEGPVVADGGTYTVAGFSTLVLISEPHHHHHH

SEQ ID NO: 246 - protein, 793 aa, synthetic construct
MPAPFAIDESTWAEATWPLGTHLTAEGLTVAVHAPAATRVQLEVYPEALGAGAQAVFLPSRGADGAWRARLGGLGAGALLGFRVWGPNWPYEEGWVPGSEAGFIADLDSEGNRFNPNKVLFDPYSREVSHTVLTDRLAELGVDDGVFGTGSDLVGGVPRRLIDTAPYAPKGVVVAEDDHVPARASAPRIPAEQAIIYEAGVRQLTGHPSVARLSDLLAGEPGFEDVVDIPPLYQGTYKGAGMLAPYLKAAGVTTVELLPVHETNASESGRPGATNAWGYMTLAFFAPNRRYAADTSWGGPTREFKEMVAAFHAAGLEVYLDVVYNHTAEGGNWNGDVATTGFTSLGGFATAEYYQMTGSKMLVDGATGTSNQMSYSSPAARQLVLDSLRYWFSQMGVDGFRFDLATVLGRLPAQSAPDDWGGLKRFFNEHPLLTEIAALAQAEGIEVIAEAWDLWGYEVGNFPRGWGEWNGRYRDAVRRFTKGDGNTGEFIDMVNGDYHHFEDNGGPQKSINFVSAHDGFTMADLVSYQTKVNDQPHPFGPSDGGSDDNMSWDSGGDQGLRRQRLRNLWVILMVSRGVPMLVAGDEMGRTQNGNNNPWALDTVAMRTNYDMVATNAPQQVPVGDPGGAYHDNLGVFGSRDERVNPLFRLATFLMRLRHRHRALHEAAWGDALAGGTDVSYLFRTPSGQGVLGEGDRALAIHVDAPGEGLWVMVNMAAEPVDFAPPPPGRGPTGDSGRDQGAGTTVWRRLVDTGVWAEPEGNFWPEGTGEVLSGTVTVGPWSVVVCQAVSAAQG

SEQ ID NO: 247 - protein, 799 aa, synthetic construct
MPAPFAIDESTWAEATWPLGTHLTAEGLTVAVHAPAATRVQLEVYPEALGAGAQAVFLPSRGADGAWRARLGGLGAGALLGFRVWGPNWPYEEGWVPGSEAGFIADLDSEGNRFNPNKVLFDPYSREVSHTVLTDRLAELGVDDGVFGTGSDLVGGVPRRLIDTAPYAPKGVVVAEDDHVPARASAPRIPAEQAIIYEAGVRQLTGHPSVARLSDLLAGEPGFEDVVDIPPLYQGTYKGAGMLAPYLKAAGVTTVELLPVHETNASESGRPGATNAWGYMTLAFFAPNRRYAADTSWGGPTREFKEMVAAFHAAGLEVYLDVVYNHTAEGGNWNGDVATTGFTSLGGFATAEYYQMTGSKMLVDGATGTSNQMSYSSPAARQLVLDSLRYWFSQMGVDGFRFDLATVLGRLPAQSAPDDWGGLKRFFNEHPLLTEIAALAQAEGIEVIAEAWDLWGYEVGNFPRGWGEWNGRYRDAVRRFTKGDGNTGEFIDMVNGDYHHFEDNGGPQKSINFVSAHDGFTMADLVSYQTKVNDQPHPFGPSDGGSDDNMSWDSGGDQGLRRQRLRNLWVILMVSRGVPMLVAGDEMGRTQNGNNNPWALDTVAMRTNYDMVATNAPQQVPVGDPGGAYHDNLGVFGSRDERVNPLFRLATFLMRLRHRHRALHEAAWGDALAGGTDVSYLFRTPSGQGVLGEGDRALAIHVDAPGEGLWVMVNMAAEPVDFAPPPPGRGPTGDSGRDQGAGTTVWRRLVDTGVWAEPEGNFWPEGTGEVLSGTVTVGPWSVVVCQAVSAAQGHHHHHH

SEQ ID NO: 248 - protein, 679 aa, synthetic construct
MTDIVTATVLGAAASRPFPLGLSAPLQGDPSDVVNVAVWAPDFAELVLYFAAPGQPWRAMTLPEFSDGVHHGVVEGMPIGSRYGFAAGDVEPEATQLLLDPYARTIDSSARLLGVRMAASFDWGNDVSPRTPWRDSVIYEAHIKGLTQLHPDIPAEIRGSYAGLAHPAMIRHLRGLGVSAVELLPIHAHADEQHLRDLGLPNYWGYNTIGYFAPQASYASAAAQAAGPQAVQDEFKGMVKLLHAAGIEVILDVVYNHTAEGKHDEPALSWRGLAENRYYRFDCDPSSGAEYLDTTGCGNTLDFSEPKVIQMALDSLRYWVEEFHIDGFRFDLAVTLVRDGANSFSPQHPFLLAATTSQALAGVKLISEPWDVGYDGWHTGQFPVGWADWNDHFRDSVRDFWIADQGSIANGGSGGGLARLADALSGSTRLFAASGRSQLASINLVTAHDGFTLADLSAYNGKHNEANGEENRDGSDNNRSWNHGVEGPSEQSDIRAARAQTSRNVMATLLLSLGVPMITAGDEMGRSQGGNNNAYCQDNEISWVDWSAVDREMLSATRRMIRLLKDYLSAQPSTYPARDESSFLHWFDVTGQPLGPEQWQDGSQRVLQMLLGSPDGLTDGLVVFNATADEVSVTLPEAEGRSFELRFSTSANYAKQQGTVITGGSQLAHSANTITIYKA

SEQ ID NO: 249 - protein, 685 aa, synthetic construct
MTDIVTATVLGAAASRPFPLGLSAPLQGDPSDVVNVAVWAPDFAELVLYFAAPGQPWRAMTLPEFSDGVHHGVVEGMPIGSRYGFAAGDVEPEATQLLLDPYARTIDSSARLLGVRMAASFDWGNDVSPRTPWRDSVIYEAHIKGLTQLHPDIPAEIRGSYAGLAHPAMIRHLRGLGVSAVELLPIHAHADEQHLRDLGLPNYWGYNTIGYFAPQASYASAAAQAAGPQAVQDEFKGMVKLLHAAGIEVILDVVYNHTAEGKHDEPALSWRGLAENRYYRFDCDPSSGAEYLDTTGCGNTLDFSEPKVIQMALDSLRYWVEEFHIDGFRFDLAVTLVRDGANSFSPQHPFLLAATTSQALAGVKLISEPWDVGYDGWHTGQFPVGWADWNDHFRDSVRDFWIADQGSIANGGSGGGLARLADALSGSTRLFAASGRSQLASINLVTAHDGFTLADLSAYNGKHNEANGEENRDGSDNNRSWNHGVEGPSEQSDIRAARAQTSRNVMATLLLSLGVPMITAGDEMGRSQGGNNNAYCQDNEISWVDWSAVDREMLSATRRMIRLLKDYLSAQPSTYPARDESSFLHWFDVTGQPLGPEQWQDGSQRVLQMLLGSPDGLTDGLVVFNATADEVSVTLPEAEGRSFELRFSTSANYAKQQGTVITGGSQLAHSANTITIYKAHHHHHH

SEQ ID NO: 250 - protein, 714 aa, synthetic construct
MDSIKVHPGRPMPYGATPVRDGINFSVFSRNGTSVILDIFKKPEDSEPYFSYEFDPVVNRTGDMWHVRLEGVETGALYLYRVDGPFAPENGHRFNKNHYLLDPYAKALTDMSIFANLPKDYAAPIDKLDVEFGKRRSARHFPKCIVIDDADFDWQGDQPLNYKLKNCVLYETHLKGFTASPTSAVAHPGTYRGMTEKIPYLKSLGITSVELMPIQEFDEFENANTNPRTGKRLKNHWGYSTISFFAPKTSYAADRTPGGAVREFKEMVREMHKNGLEVILDIVFNHTAEGNEHGLTLNFRGFDNSIYYILEDKHKQYYKNFSGCGNTVNCNHPVVRSFIIDCLRYWVIEMHVDGFRFDLASILGRDRNGNLIKEPPVLERIAEDPILGRTKIIAEAWDAGGAYQVGTFPGGRWAEWNDRFRDEIRRFWRGDDFLCTAAATRMTGSADLYQDDGRKPYHSVNFITSHDGFTLNDLVSYNGKHNEENGEHNRDGSDNNSSYNYGYEGPTANKAIEGIRTRQVKNMLLTLLLSQGTPMLLSGDEFRRTQGGNNNAYCQDNELSWLNWTNQETYAEIVTFLRKAIHTRLTHPVFRRPDFFEGQDHSANLLPDINWFASDGKTPDWSQLNHFLAFRLDGSKAEIFADRDDNDFFIMCNTGATDMTVKTPSLNKGKKWYRFIDTSVPAPNDFTDHGSEELLEEQKTYILPARTMAVLLGK

SEQ ID NO: 251 - protein, 720 aa, synthetic construct
MDSIKVHPGRPMPYGATPVRDGINFSVFSRNGTSVILDIFKKPEDSEPYFSYEFDPVVNRTGDMWHVRLEGVETGALYLYRVDGPFAPENGHRFNKNHYLLDPYAKALTDMSIFANLPKDYAAPIDKLDVEFGKRRSARHFPKCIVIDDADFDWQGDQPLNYKLKNCVLYETHLKGFTASPTSAVAHPGTYRGMTEKIPYLKSLGITSVELMPIQEFDEFENANTNPRTGKRLKNHWGYSTISFFAPKTSYAADRTPGGAVREFKEMVREMHKNGLEVILDIVFNHTAEGNEHGLTLNFRGFDNSIYYILEDKHKQYYKNFSGCGNTVNCNHPVVRSFIIDCLRYWVIEMHVDGFRFDLASILGRDRNGNLIKEPPVLERIAEDPILGRTKIIAEAWDAGGAYQVGTFPGGRWAEWNDRFRDEIRRFWRGDDFLCTAAATRMTGSADLYQDDGRKPYHSVNFITSHDGFTLNDLVSYNGKHNEENGEHNRDGSDNNSSYNYGYEGPTANKAIEGIRTRQVKNMLLTLLLSQGTPMLLSGDEFRRTQGGNNNAYCQDNELSWLNWTNQETYAEIVTFLRKAIHTRLTHPVFRRPDFFEGQDHSANLLPDINWFASDGKTPDWSQLNHFLAFRLDGSKAEIFADRDDNDFFIMCNTGATDMTVKTPSLNKGKKWYRFIDTSVPAPNDFTDHGSEELLEEQKTYILPARTMAVLLGKHHHHHH

SEQ ID NO: 252 - protein, 827 aa, synthetic construct
MVEGCSTGERGPPVALFATALGVVPIMMTSIRNLGRAWSALPWRPLLAVGLGAALSACGASDPAAALLEEGPPVLGGQEQQAVSWTLGARYDASKSNISFQVYSKNATRIDLYIYATAYGAQEVVSYEMTTVPDTGLWSKTVSVATLQNTYKITGPVYYGYRAWGKNWPFSTAWTKGSAAGFIADVDGSGNRFNPNKVLLDPYALEISHDPSNPQSTDGSVFASGPGTRNIDSGPRVPKGIVLAGDSQSIGTRPTRAFKDDVIYEVHVRGLTRNDSSIDPAYRGTYKGAGLKAPALAALGVTAVEFLPLHETENDANDNAVGTPGDNYWGYMSLNYFAPDRRYAYDTKAGGPTREFKEMVKAFHDNGIKVFVDVVYNHTGEGGAWKAGDSSTYNVISFRGLDNATYYSLTGDKQFNWDNTGVGGNYNTYNPKAQDLIIHSLAYWKDTLGVDGFRFDLASVLGNAQEHGGFNFVRDNSNTALNRIIRDLGPRPGSGGAGTDFIAEPWAIGGNSYQVGNFPAGWAEWNGIFRDTFRKDQNQLGVETVTPGQLATRFTGSADLYGEGNGDTRKPYHSVNFMVAHDGFTLKDLYSCNSKNNNQPWPYGPSDGGDDNNHSWDQGGNAADQRKAARNGFAFLMLSAGVPMFNGGDEFLRSQACNNNAYNLDSDKNWLNYSLSADQTKFKTFAQRLIAFRKAHPALRPANFYLSTDTNGNVMEQHRWFKPDGYVPDAGYFNNGSNHAIAFRIDGTEFNDSASALYVAYNGWSDNVNFNLPWPGNGKNWYRVADTCPWAETDGVNAVAPGAETLLGGEGYSYGLCGRGVLLLIAK

SEQ ID NO: 253 - protein, 833 aa, synthetic construct
MVEGCSTGERGPPVALFATALGVVPIMMTSIRNLGRAWSALPWRPLLAVGLGAALSACGASDPAAALLEEGPPVLGGQEQQAVSWTLGARYDASKSNISFQVYSKNATRIDLYIYATAYGAQEVVSYEMTTVPDTGLWSKTVSVATLQNTYKITGPVYYGYRAWGKNWPFSTAWTKGSAAGFIADVDGSGNRFNPNKVLLDPYALEISHDPSNPQSTDGSVFASGPGTRNIDSGPRVPKGIVLAGDSQSIGTRPTRAFKDDVIYEVHVRGLTRNDSSIDPAYRGTYKGAGLKAPALAALGVTAVEFLPLHETENDANDNAVGTPGDNYWGYMSLNYFAPDRRYAYDTKAGGPTREFKEMVKAFHDNGIKVFVDVVYNHTGEGGAWKAGDSSTYNVISFRGLDNATYYSLTGDKQFNWDNTGVGGNYNTYNPKAQDLIIHSLAYWKDTLGVDGFRFDLASVLGNAQEHGGFNFVRDNSNTALNRIIRDLGPRPGSGGAGTDFIAEPWAIGGNSYQVGNFPAGWAEWNGIFRDTFRKDQNQLGVETVTPGQLATRFTGSADLYGEGNGDTRKPYHSVNFMVAHDGFTLKDLYSCNSKNNNQPWPYGPSDGGDDNNHSWDQGGNAADQRKAARNGFAFLMLSAGVPMFNGGDEFLRSQACNNNAYNLDSDKNWLNYSLSADQTKFKTFAQRLIAFRKAHPALRPANFYLSTDTNGNVMEQHRWFKPDGYVPDAGYFNNGSNHAIAFRIDGTEFNDSASALYVAYNGWSDNVNFNLPWPGNGKNWYRVADTCPWAETDGVNAVAPGAETLLGGEGYSYGLCGRGVLLLIAKHHHHHH

SEQ ID NO: 254 - protein, 846 aa, synthetic construct
MKNPPLHRYATRPGLYFTDDGGADVVVRSETADQVWLCVLEPIDEPSAFFQDAIRLFEDPNASFIQQIHEFPVCTRIIEHLYLRETLFRMTGPNYGLWYVHLPKAWDGMRYGYRVDGAWDPKHGVRFNPYKFLLDPYGKGIDGSMELTPAAFSYECDVVDHKVTGSAYGAMSTVDSLGHVPVSVAIDDRDIHKHEGDPQHPHVPWRKTVIYEMHVKGFTANAPWLPEALRGTYAGLAHPTTLAYLQGLGITSIELLPIMAKQDELFLQEHGRKNYWGYSTLSYFAPEPSYATKAAQEKGAAAVRQEVIDMVRALHEAGFEVIMDVVYNHTCEGGVEGPTVCWRGLDDLAYYRHQKSNTGRLEDTTGCGNTLDFTNTHVVTFAIDSLRYWAKRIGIDGFRFDLGVTLARLEGEFTHHHPFLYALRSDLLLGNLKLIMEPWDLGNLGWRTGQFSVPFAEWNDRFRDTARTFWLEDVDGGSDFGRISLQEMATRLCGSADLFATEPGRGAPASINFVSCHDGFTLTDLTRYRSKHNEANGENNNDGSSVNHSANFGAEGVTDDPDVITAREQAAMNMIGMLLLSLGTPMMLAGDEFRNTQDGNNNAYCQDNDITWLKWDWMYSTNKTREMRRLKSVSRLVALRKSLDLYHHEDFFTRLTQLGLLKPSSRVQWFLPDGTTPMERDWFDLGVRSFTMRLLSNSEVDVCIVVNGTADDRTFRLPPDTHWTPKWCSAEINGRRAGHGIQVEECDLNDDTTVWTQHVPDVSEMVRTLVEEVAMQRTESSTEDEADTIEFAMPATDHAANGTSSAPRDEVSADMPDAPVDDTPDTPVDDNVWTMPALSITLMKQV

SEQ ID NO: 255 - protein, 852 aa, synthetic construct
MKNPPLHRYATRPGLYFTDDGGADVVVRSETADQVWLCVLEPIDEPSAFFQDAIRLFEDPNASFIQQIHEFPVCTRIIEHLYLRETLFRMTGPNYGLWYVHLPKAWDGMRYGYRVDGAWDPKHGVRFNPYKFLLDPYGKGIDGSMELTPAAFSYECDVVDHKVTGSAYGAMSTVDSLGHVPVSVAIDDRDIHKHEGDPQHPHVPWRKTVIYEMHVKGFTANAPWLPEALRGTYAGLAHPTTLAYLQGLGITSIELLPIMAKQDELFLQEHGRKNYWGYSTLSYFAPEPSYATKAAQEKGAAAVRQEVIDMVRALHEAGFEVIMDVVYNHTCEGGVEGPTVCWRGLDDLAYYRHQKSNTGRLEDTTGCGNTLDFTNTHVVTFAIDSLRYWAKRIGIDGFRFDLGVTLARLEGEFTHHHPFLYALRSDLLLGNLKLIMEPWDLGNLGWRTGQFSVPFAEWNDRFRDTARTFWLEDVDGGSDFGRISLQEMATRLCGSADLFATEPGRGAPASINFVSCHDGFTLTDLTRYRSKHNEANGENNNDGSSVNHSANFGAEGVTDDPDVITAREQAAMNMIGMLLLSLGTPMMLAGDEFRNTQDGNNNAYCQDNDITWLKWDWMYSTNKTREMRRLKSVSRLVALRKSLDLYHHEDFFTRLTQLGLLKPSSRVQWFLPDGTTPMERDWFDLGVRSFTMRLLSNSEVDVCIVVNGTADDRTFRLPPDTHWTPKWCSAEINGRRAGHGIQVEECDLNDDTTVWTQHVPDVSEMVRTLVEEVAMQRTESSTEDEADTIEFAMPATDHAANGTSSAPRDEVSADMPDAPVDDTPDTPVDDNVWTMPALSITLMKQVHHHHHH

SEQ ID NO: 256 - protein, 672 aa, synthetic construct
MVSLVQTRESSGKSKPYGSKRDSKGVNFAIYSRLATEAALCLFHFDDRRPFKEIPLDPQINRTGYVWHIYVENLPRRLCYAYRFKKGKGKVFTDYYDYQRLVIDPYAKGLATSSVWGEGIGEMPLGLVDEELIFDWEGDRPLNLPREEMMIYEMHIRGFTNHSSSNALWRGKFLGAVEKIPYLKSLGVNAVKLMPINEFNELEYFRYNPLNGEKLVNYWGYSPLHYFSPMNRYASIDEFGQSILDFKTMVKEFHRNGIEVILDIVLNHTGESDQEPFSFFGIDPQTYYLFDDQHEKMDFTGCGNTINSNHPIVRDFIKDCLRYWVSEMHVDGFRFDLAGVMFRGVHGEPLKNPPLIDAISNDPILAATKLIAEPWDAAGLYLLGKFYPREERWSEWNDVYRDVVRQFIKGDKGKNRSFATRLCGSDDIFGRSRTPRSSVNFISAHDGFTLRDLVTYNQKDNTSNGENNRDGHPANFSWNCGEEGETDDQEINDLRVRQMKNFHLALMLSQGIPMLLMGNEYGHTRFGNNNSWCQDNEMNWFLWNELELQGDFFRFYRMCIQFRARHPQLRRGRYLTPEDIVWHGKQPDHPDWDGDTQFLAYLLVDEVQSHHLFAAYNPSAENKEVTLPQGEWKLIADTSLSSPDDFRDEEEAPFLPSQEYLLKPYSIALLQC

SEQ ID NO: 257 - protein, 678 aa, synthetic construct
MVSLVQTRESSGKSKPYGSKRDSKGVNFAIYSRLATEAALCLFHFDDRRPFKEIPLDPQINRTGYVWHIYVENLPRRLCYAYRFKKGKGKVFTDYYDYQRLVIDPYAKGLATSSVWGEGIGEMPLGLVDEELIFDWEGDRPLNLPREEMMIYEMHIRGFTNHSSSNALWRGKFLGAVEKIPYLKSLGVNAVKLMPINEFNELEYFRYNPLNGEKLVNYWGYSPLHYFSPMNRYASIDEFGQSILDFKTMVKEFHRNGIEVILDIVLNHTGESDQEPFSFFGIDPQTYYLFDDQHEKMDFTGCGNTINSNHPIVRDFIKDCLRYWVSEMHVDGFRFDLAGVMFRGVHGEPLKNPPLIDAISNDPILAATKLIAEPWDAAGLYLLGKFYPREERWSEWNDVYRDVVRQFIKGDKGKNRSFATRLCGSDDIFGRSRTPRSSVNFISAHDGFTLRDLVTYNQKDNTSNGENNRDGHPANFSWNCGEEGETDDQEINDLRVRQMKNFHLALMLSQGIPMLLMGNEYGHTRFGNNNSWCQDNEMNWFLWNELELQGDFFRFYRMCIQFRARHPQLRRGRYLTPEDIVWHGKQPDHPDWDGDTQFLAYLLVDEVQSHHLFAAYNPSAENKEVTLPQGEWKLIADTSLSSPDDFRDEEEAPFLPSQEYLLKPYSIALLQCHHHHHH

SEQ ID NO: 258 - protein, 621 aa, synthetic construct
MNPSFTTGSKLPLGSSPTEKGINFAVYSHHATNIKLRLFEIGQKTPFAEFPMERSDDYWHLCVTNLPYTFEYTYQAEGPYDPSKGLLFCKEMDLVDPYARAVNASDTWGNHHTPMRALYETKMPFDWEHTSRPMIPAEDLIIYEMHVRSFTMHPSSGSKNPGTFLGMIEKIPYLKKLGINAVELMPIHEFNETENLRRSPGTGGKLFNYWGYSTSNFFAPMRRFGKEEDLKFLIRELHREGIEVILDVVYNHTSEGNDQNYYHSFRGLDNPTYYIIDENGYHNYTGCGNTLKCQHPVVQDFILDSLRYWVTEFHVDGFRFDLASIMTRGEDGKPIQDPPLIKRIASDPILAPTKMIAEPWDPAGLYQVGTFPSWRFAEWNGKFRDDVRKFIRGDGNIEAMKNRLLGSPDVYTEKGTPQHSINFITVHDGFTLHDLVSYNEKHNEQNGEQNQDGANDNESWNCGVEGKTTDQAILNLRLQQMRNFMVALFIAQGIPMLLMGDEYAHTREGNNNAYCQDNELNYFLWDQASPLFEFIQKLIALRKSHPIFRQKTFPSAVKWEEKDYLGLTLGEELFIAFNPSAQEYQLEKEGWEILLSTASQVQTLDKLQPYTSVLFGKKKAL

SEQ ID NO: 259 - protein, 627 aa, synthetic construct
MNPSFTTGSKLPLGSSPTEKGINFAVYSHHATNIKLRLFEIGQKTPFAEFPMERSDDYWHLCVTNLPYTFEYTYQAEGPYDPSKGLLFCKEMDLVDPYARAVNASDTWGNHHTPMRALYETKMPFDWEHTSRPMIPAEDLIIYEMHVRSFTMHPSSGSKNPGTFLGMIEKIPYLKKLGINAVELMPIHEFNETENLRRSPGTGGKLFNYWGYSTSNFFAPMRRFGKEEDLKFLIRELHREGIEVILDVVYNHTSEGNDQNYYHSFRGLDNPTYYIIDENGYHNYTGCGNTLKCQHPVVQDFILDSLRYWVTEFHVDGFRFDLASIMTRGEDGKPIQDPPLIKRIASDPILAPTKMIAEPWDPAGLYQVGTFPSWRFAEWNGKFRDDVRKFIRGDGNIEAMKNRLLGSPDVYTEKGTPQHSINFITVHDGFTLHDLVSYNEKHNEQNGEQNQDGANDNESWNCGVEGKTTDQAILNLRLQQMRNFMVALFIAQGIPMLLMGDEYAHTREGNNNAYCQDNELNYFLWDQASPLFEFIQKLIALRKSHPIFRQKTFPSAVKWEEKDYLGLTLGEELFIAFNPSAQEYQLEKEGWEILLSTASQVQTLDKLQPYTSVLFGKKKALHHHHHH

SEQ ID NO: 260 - protein, 686 aa, synthetic construct
MRSWHTMEGWHDPPGVSWIEEEKAWNFVLYAREATRVVLLIYSREDQTKPILEYQFDQYRNKSGPVWHCRISHDRAPKAHYYAYRVSGPLHEEDKFNRFDPEKILLDPYAKAVYFPPGFDREVAKHPGPNDGKAPLGVIDAEIHFDWKDDRFIRHDSDLVIYEMHVRGFTKHSSSQVPTAKRGTYAGVIEKIPYLRDLGVTAVELMPIFQYDPQELNYWGYMPLNFFAPHCDYAVNSESQQVREEFREMVQELHRAGIEVILDVVYNHTCEGDDNGPCYSFKGIGNSMYYMEAPDENGEPTFANYSGCGNTLNANTLAVRKLVVDSLKYWRDEMHVDGFRFDLASIFARQNDGTISAGQTPIFSQIVTAEDFLNVRLIAEPWDAAGTYQLGHSFPGWLWMQWNGRYRDTMQQFVAGQPGMIGDLMSRLYGSADLFPDDLNHSCRPWQSVNYITSHDGSTLYDMVTYEGKYNWDNGEENRDGSHEFKWNCGHEGEEGTPPEVMRLRKRQVKNFMTLLMLSNGSAMFRMGDEFMATQKGNNNAYNQDNETSWLDWTRLDRFRDVYRFVKLLIAFRKSHPSLSRSHYWRNDIRWYGPDGPCDISHNSHTLAYSLRGSSVNDQDLYVMINGSREPRSFEICDGAETQWKRILDTALDSPDDIHGVSGYETLNQTSYHLEANSIVVLVRDA

SEQ ID NO: 261 - protein, 692 aa, synthetic construct
MRSWHTMEGWHDPPGVSWIEEEKAWNFVLYAREATRVVLLIYSREDQTKPILEYQFDQYRNKSGPVWHCRISHDRAPKAHYYAYRVSGPLHEEDKFNRFDPEKILLDPYAKAVYFPPGFDREVAKHPGPNDGKAPLGVIDAEIHFDWKDDRFIRHDSDLVIYEMHVRGFTKHSSSQVPTAKRGTYAGVIEKIPYLRDLGVTAVELMPIFQYDPQELNYWGYMPLNFFAPHCDYAVNSESQQVREEFREMVQELHRAGIEVILDVVYNHTCEGDDNGPCYSFKGIGNSMYYMEAPDENGEPTFANYSGCGNTLNANTLAVRKLVVDSLKYWRDEMHVDGFRFDLASIFARQNDGTISAGQTPIFSQIVTAEDFLNVRLIAEPWDAAGTYQLGHSFPGWLWMQWNGRYRDTMQQFVAGQPGMIGDLMSRLYGSADLFPDDLNHSCRPWQSVNYITSHDGSTLYDMVTYEGKYNWDNGEENRDGSHEFKWNCGHEGEEGTPPEVMRLRKRQVKNFMTLLMLSNGSAMFRMGDEFMATQKGNNNAYNQDNETSWLDWTRLDRFRDVYRFVKLLIAFRKSHPSLSRSHYWRNDIRWYGPDGPCDISHNSHTLAYSLRGSSVNDQDLYVMINGSREPRSFEICDGAETQWKRILDTALDSPDDIHGVSGYETLNQTSYHLEANSIVVLVRDAHHHHHH

SEQ ID NO: 262 - protein, 652 aa, synthetic construct
MYGSSVGQSVIGCHFTEMRTHLGVYTTIASKMLLEIYEEARSIVPITKVIMDAATYQKDNVFSIQIEGLKEGMAYVWRIVHEDYSYSPSIMDPYAKGTFFFQGEWRNIIKKTNRYHLPKPQIPWEETILYEMHVGHFTMNNKTLSNEKRGTFIGLMQQLPYLKQLGVTTLELLPIFKWNAYTLKNRNPQTGELLEDVWGYNPVGFFCVDERFSVSKESSKAIDEFKLLVEKAHEEGLEIILDVVYNHTGEGGEDGTAFHFKYLAPKVYYKYNDKGQFLNCSGTGNTLNTNHSIVKKFIIDSLVYWSEEVGVDGFRFDLASILGQDECGRWIKTSLLNEIAEHPLLGKVKLISESWDAKGSYDVGRMPYPFREWSDYFRDTMRQFVKGDQGKIKALADCIQGKEVYFTDLTKGTSHMIHFITAHDGFTMWDLLSYNDKHNEANGEGNRDGHNANYSYNWGVEGTTEDTNILEARKRGMRNLMCLLILAQGVPMLLMGDEIGRTQGGNNNAFCQNNESVWMDWERVTTFKSQYLFVQRLIALRKSLDYFKTADKNNYKISWHGIRYNQPDWSYYSKSIACFIEGDQSLFLVANSHYESLRFEMPPSNKKWCRVIDTAYKEIEDIINEEIIEETNYEVKPYSICFFKEINHKKIG

SEQ ID NO: 263 - protein, 658 aa, synthetic construct
MYGSSVGQSVIGCHFTEMRTHLGVYTTIASKMLLEIYEEARSIVPITKVIMDAATYQKDNVFSIQIEGLKEGMAYVWRIVHEDYSYSPSIMDPYAKGTFFFQGEWRNIIKKTNRYHLPKPQIPWEETILYEMHVGHFTMNNKTLSNEKRGTFIGLMQQLPYLKQLGVTTLELLPIFKWNAYTLKNRNPQTGELLEDVWGYNPVGFFCVDERFSVSKESSKAIDEFKLLVEKAHEEGLEIILDVVYNHTGEGGEDGTAFHFKYLAPKVYYKYNDKGQFLNCSGTGNTLNTNHSIVKKFIIDSLVYWSEEVGVDGFRFDLASILGQDECGRWIKTSLLNEIAEHPLLGKVKLISESWDAKGSYDVGRMPYPFREWSDYFRDTMRQFVKGDQGKIKALADCIQGKEVYFTDLTKGTSHMIHFITAHDGFTMWDLLSYNDKHNEANGEGNRDGHNANYSYNWGVEGTTEDTNILEARKRGMRNLMCLLILAQGVPMLLMGDEIGRTQGGNNNAFCQNNESVWMDWERVTTFKSQYLFVQRLIALRKSLDYFKTADKNNYKISWHGIRYNQPDWSYYSKSIACFIEGDQSLFLVANSHYESLRFEMPPSNKKWCRVIDTAYKEIEDIINEEIIEETNYEVKPYSICFFKEINHKKIGHHHHHH

SEQ ID NO: 264 - protein, 692 aa, synthetic construct
MRSPRYRVRPGSWDTAGATVSNDGVNFCVFSRYAERMSLLLFERETSKEPYEVLHLNPRINRTFFFWHIFVENLPDGTYYNWRATGPGDTSETGSRLDSEKALLDPWATTISDRLWDRATACRPGNNVAKAMRAQVIRDDYDWEEDQPLYVSLNNAIIYEMHVGGFTRHPSSGVVHPGTFEGVTEKIPYLQDLGITHVELMPVMAFDPQDVPPQTAKMGMENYWGYSTHSFFAPHPGFAVTAINARDEFRDMVKAFHRAGIGVILDVVFNHTAEGGKNGPVISFKAFGNEIFYHLDFEDRSLYRDYTGCGNTMNCNHPMVTRFLIDALLYWVRHMHVDGFRFDLASALARGEDGNPHHHAPVLWAIELSPSLNRAHIIAEAWDAAGLYQVGDFPGYRWAEWNGRYRDVIRSFVRGDAGMVPEVATRMSGSSDMYEGRGRLPMNSINFITCHDGFTLCDLVSYNAKHNEANGEDNRDGHDHNLSWNCGIEGPTDDPEIQALRRRQARNFISILMLSQGVPMLLSGDEVFRSKSGNNNTYCQNNELSWSDWDLVDTNSEMLEFVRSMIALRRRHPTLTRDRFLRGKPDYGHSRPDIIWHGVDLEQPNWTDATSRQLAFTLEGSVDDEQPLHVMLNMGSEPIAFALPPIAGLQWGLALDTSSERPAVEPADQRPIAEDRLQLGPFSVVVLEARPA

SEQ ID NO: 265 - protein, 698 aa, synthetic construct
MRSPRYRVRPGSWDTAGATVSNDGVNFCVFSRYAERMSLLLFERETSKEPYEVLHLNPRINRTFFFWHIFVENLPDGTYYNWRATGPGDTSETGSRLDSEKALLDPWATTISDRLWDRATACRPGNNVAKAMRAQVIRDDYDWEEDQPLYVSLNNAIIYEMHVGGFTRHPSSGVVHPGTFEGVTEKIPYLQDLGITHVELMPVMAFDPQDVPPQTAKMGMENYWGYSTHSFFAPHPGFAVTAINARDEFRDMVKAFHRAGIGVILDVVFNHTAEGGKNGPVISFKAFGNEIFYHLDFEDRSLYRDYTGCGNTMNCNHPMVTRFLIDALLYWVRHMHVDGFRFDLASALARGEDGNPHHHAPVLWAIELSPSLNRAHIIAEAWDAAGLYQVGDFPGYRWAEWNGRYRDVIRSFVRGDAGMVPEVATRMSGSSDMYEGRGRLPMNSINFITCHDGFTLCDLVSYNAKHNEANGEDNRDGHDHNLSWNCGIEGPTDDPEIQALRRRQARNFISILMLSQGVPMLLSGDEVFRSKSGNNNTYCQNNELSWSDWDLVDTNSEMLEFVRSMIALRRRHPTLTRDRFLRGKPDYGHSRPDIIWHGVDLEQPNWTDATSRQLAFTLEGSVDDEQPLHVMLNMGSEPIAFALPPIAGLQWGLALDTSSERPAVEPADQRPIAEDRLQLGPFSVVVLEARPAHHHHHH

SEQ ID NO: 266 - protein, 708 aa, synthetic construct
MVLDRIDIHPTHEYKGFKLRYGRPFPFGATLVPGGVNFSIFSRHATACTLVLFERHAKEPFAEIPFLEEFRIGNVFTMTVFDLNYEELEYGYRMDGPYDPKEGHWFDKTKILMDPYARIIGGRDIWGKQPDWDDVYHHRARIAFDDFDWESDRPLEIPPEDLTVYEMHVRSFTKHESSGVKHPGTYAAIRDKIPYLKELGVNCIELMPIYEFDEFENSRPNPQNPEETLMNYWGYSTVGFFAPKAGYAATGKFGMQVDELKAMIKELHRNGIEVILDVVFNHTAEGNEKGPYISYRGLDNQTYYMLTPDGYYFNFSGTGNTLNCNNPIVRNMVLDCLRYWAAEYHVDGFRFDLASILSRDAWGAPLANPPLLETLAFDPILAKCKLIAEAWDAGGLYQVGSFPAFGRWAEWNGKYRDCIRKFIKGDDGMAGEMAQRIQGSPDLYAWGGRGPATSINFITCHDGFTLMDTVSYNGKHNDANGEDNRDGNNDNDSWNCGWEGPTDDSGINALRQRQIKNAVAIMIASQGVPMFLMGDEMGRTKYGNNNTYCHDNELNWLDWDLLNQNQGLFRFFKLAIAFRMAHPVLRSRTHFRNYDYVGSGYSDITWHGTQAWNADWSDSSHALAFMLCGKHAKEGTVNDDYIYVAMNTHWDALWFEPPGLPEGMQWHVFANTGASSPEDVWTPGQEPALENQHGILLGDRSVVILVGR

SEQ ID NO: 267 - protein, 714 aa, synthetic construct
MVLDRIDIHPTHEYKGFKLRYGRPFPFGATLVPGGVNFSIFSRHATACTLVLFERHAKEPFAEIPFLEEFRIGNVFTMTVFDLNYEELEYGYRMDGPYDPKEGHWFDKTKILMDPYARIIGGRDIWGKQPDWDDVYHHRARIAFDDFDWESDRPLEIPPEDLTVYEMHVRSFTKHESSGVKHPGTYAAIRDKIPYLKELGVNCIELMPIYEFDEFENSRPNPQNPEETLMNYWGYSTVGFFAPKAGYAATGKFGMQVDELKAMIKELHRNGIEVILDVVFNHTAEGNEKGPYISYRGLDNQTYYMLTPDGYYFNFSGTGNTLNCNNPIVRNMVLDCLRYWAAEYHVDGFRFDLASILSRDAWGAPLANPPLLETLAFDPILAKCKLIAEAWDAGGLYQVGSFPAFGRWAEWNGKYRDCIRKFIKGDDGMAGEMAQRIQGSPDLYAWGGRGPATSINFITCHDGFTLMDTVSYNGKHNDANGEDNRDGNNDNDSWNCGWEGPTDDSGINALRQRQIKNAVAIMIASQGVPMFLMGDEMGRTKYGNNNTYCHDNELNWLDWDLLNQNQGLFRFFKLAIAFRMAHPVLRSRTHFRNYDYVGSGYSDITWHGTQAWNADWSDSSHALAFMLCGKHAKEGTVNDDYIYVAMNTHWDALWFEPPGLPEGMQWHVFANTGASSPEDVWTPGQEPALENQHGILLGDRSVVILVGRHHHHHH

SEQ ID NO: 268 - protein, 689 aa, synthetic construct
MGKIHKGSPWPLGSSITSRGVNFSVASPEASYIELLIFKNEDDLQPKKILTLDKNHRSGDYWHIEVEGINTGCFYCYRVIGNRSTCKQDIFSRKILLDPCSRGIAGWNIFQRESATGLSTNIDKCLKSIVTERDQFDFQSYPRPKHSWDKTIIYELHVGGFTKSSESDVKDKIKGTFLGLIEKIPYLKQLGVTTLELLPVFAFDTTDSPYGLNNYWGYSPINWFTPHHSFIASNNPINARDQFRNFIKVCHKNDLEVILDVVYNHTTEGNEKGPIISWKGFAESTYYHQNKEGKYLDVTGCGNTIAANNPLVRQLILESMRCWANELGVDGFRFDLGISLSRGKDLKPLDSPPLFEEIESDPALSDLKLISEPWDCGGLYRLSDFPAKRCCTWNGHFRDDIRRFWNGDKNSTWPLKDRLTGSPELYKDNFKSAQKSINFITSHDGFTLKDLVSFNLKHNLSNGESNRDGENHNNSCNNGIEGPTTNKKVNLIRSKNQRNLIATLLLSPGIPMILMGDEVGRSQGGNNNAWCQDNPLGWMIWRTDNCDNELRSFVSMCIYIRKELSDFFAPLININSDSPSLQSQEKLWVQWHGVKINAPDWGSWSNTIGFSINKAKEGAIIWMGFNAFNQSMKFELPKPLSPWVKILDTTLLTQKEYGLFRLSNQLEIEIESKSLVVLVAKEYTKKLRI

SEQ ID NO: 269 - protein, 695 aa, synthetic construct
MGKIHKGSPWPLGSSITSRGVNFSVASPEASYIELLIFKNEDDLQPKKILTLDKNHRSGDYWHIEVEGINTGCFYCYRVIGNRSTCKQDIFSRKILLDPCSRGIAGWNIFQRESATGLSTNIDKCLKSIVTERDQFDFQSYPRPKHSWDKTIIYELHVGGFTKSSESDVKDKIKGTFLGLIEKIPYLKQLGVTTLELLPVFAFDTTDSPYGLNNYWGYSPINWFTPHHSFIASNNPINARDQFRNFIKVCHKNDLEVILDVVYNHTTEGNEKGPIISWKGFAESTYYHQNKEGKYLDVTGCGNTIAANNPLVRQLILESMRCWANELGVDGFRFDLGISLSRGKDLKPLDSPPLFEEIESDPALSDLKLISEPWDCGGLYRLSDFPAKRCCTWNGHFRDDIRRFWNGDKNSTWPLKDRLTGSPELYKDNFKSAQKSINFITSHDGFTLKDLVSFNLKHNLSNGESNRDGENHNNSCNNGIEGPTTNKKVNLIRSKNQRNLIATLLLSPGIPMILMGDEVGRSQGGNNNAWCQDNPLGWMIWRTDNCDNELRSFVSMCIYIRKELSDFFAPLININSDSPSLQSQEKLWVQWHGVKINAPDWGSWSNTIGFSINKAKEGAIIWMGFNAFNQSMKFELPKPLSPWVKILDTTLLTQKEYGLFRLSNQLEIEIESKSLVVLVAKEYTKKLRIHHHHHH

SEQ ID NO: 270 - protein, 788 aa, synthetic construct
MQIEMGSSYDKDTGKVHFNVFSKNASHIEIYFYLTPFGADEVMKKSLVRNNDIWSLDLPLAELKSAGFTSNCVYYGYRTWGPNWEYMEDWNKTSDKGFICDVDDEGNRFNPNKLLIDPYSKELSHDPQVAKIYIDPNVYIDNYYSGANRNADTGKIAPKSMLFLEKDKTSYGIKPKRHLKDDIIYEVNLRGLTMMDKSIPKDERGTYKAASIKAAYLKELGVTAVEFLPIHEFADEQNDDNDPRGDNYWGYMTLNFFSPNRRYSFDKSPGGPTREFKQMIKAFHEQGIKVFLDVVYNHTGEGILKRIINQGNPSTEEDIKKAIQEANHSRGDDNLQDYTAACIMSFTGLDNSNYYYLRDGNKRYEGRGGCGGNLKYDNEVVRKLIYDSLKYWKDEMGVDGFRFDLAPVLSVTGSNGNYWPDTNTTIFEEISNILPSRIKDQDNGADLIAEPWGEGSSIDWLDKFPNSWAVWNKAFRDIFKTSMNKYGVAPFRICNIANVSSGSSSIIKKKPWNSINYFVSHDDCNSLRNIFSYNEYFHLNEAGIKNDQITWNQGNDPSLQKKAVLNAFTLMMFSAGVPMFTGGDEFYRAISPYHQGVGRMNMVTVDGPDGYVDFQHFNKLTVLRNSGNSHEAEQLTKNTDELYIFEFVKKVIDFRSNHECLRPENYFKGERENDNFLKDITWYQHNGLEIDSSYWDSSDFVAYRISAESEKVANVNNRIFSIYLAYNRSPRTEKVLLPSNIPNKRWYRLIDTDNTYGWMSELRNFDGDTLLENEYVMHDRSILVLIEK

SEQ ID NO: 271 - protein, 794 aa, synthetic construct
MQIEMGSSYDKDTGKVHFNVFSKNASHIEIYFYLTPFGADEVMKKSLVRNNDIWSLDLPLAELKSAGFTSNCVYYGYRTWGPNWEYMEDWNKTSDKGFICDVDDEGNRFNPNKLLIDPYSKELSHDPQVAKIYIDPNVYIDNYYSGANRNADTGKIAPKSMLFLEKDKTSYGIKPKRHLKDDIIYEVNLRGLTMMDKSIPKDERGTYKAASIKAAYLKELGVTAVEFLPIHEFADEQNDDNDPRGDNYWGYMTLNFFSPNRRYSFDKSPGGPTREFKQMIKAFHEQGIKVFLDVVYNHTGEGILKRIINQGNPSTEEDIKKAIQEANHSRGDDNLQDYTAACIMSFTGLDNSNYYYLRDGNKRYEGRGGCGGNLKYDNEVVRKLIYDSLKYWKDEMGVDGFRFDLAPVLSVTGSNGNYWPDTNTTIFEEISNILPSRIKDQDNGADLIAEPWGEGSSIDWLDKFPNSWAVWNKAFRDIFKTSMNKYGVAPFRICNIANVSSGSSSIIKKKPWNSINYFVSHDDCNSLRNIFSYNEYFHLNEAGIKNDQITWNQGNDPSLQKKAVLNAFTLMMFSAGVPMFTGGDEFYRAISPYHQGVGRMNMVTVDGPDGYVDFQHFNKLTVLRNSGNSHEAEQLTKNTDELYIFEFVKKVIDFRSNHECLRPENYFKGERENDNFLKDITWYQHNGLEIDSSYWDSSDFVAYRISAESEKVANVNNRIFSIYLAYNRSPRTEKVLLPSNIPNKRWYRLIDTDNTYGWMSELRNFDGDTLLENEYVMHDRSILVLIEKHHHHHH

SEQ ID NO: 272 - protein, 704 aa, synthetic construct
MDPVTFTTGNAVIDRTRGNSFPLGATVLQGGVNFSVFSKSATFVELLLFDHAEDPGPSRTILLDPRKNRTYHYWHVFVPDVGPGQIYGYRVHGPYEPARGMRFDPGKVLLDPYGRAVVVPEKYSRTAAGLPGDNCGAAMKSVVTDPRTYDWEGDLPLRRPFTRTIIYEMHVRGFTAHPNSGVAPEKRGTYAGLIEKIPYLKDLGITAVELLPVYHFDEQDAPPEFKNYWGYAPVSFFTPHPAYSSRKEPLCVVDEFRDMIKALHRAGIEVILDVVYNHTAEDNHEGPTLSFRGFENDAYYILGPDKRHYSNYTGCGNTLNASNPFVRRMIIDSLHYWVQEMHVDGFRFDLASILARDEQGRPLANPPVLWDIETDPVLSGVKLIAEAWDAAGLYQVGSFIGDSWKEWNVRFRDDVRSFLKGDRNTVSKFASRLLGSPDIYGHEEREPEQSINFVTCHDGFTLNDLVSYNDKHNEENGEENRDGSNDNMSWNCGIEGPTDALPIERLRNRQVKNFFAVTLLAAGAPMLLMGDEVRRTQRGNNNAYGQDNEISWFDWGLVEKHANVLRFVKYLITARLRRNVSLEDLGLTLNQLLSQAKITWHGVKLNQPDWGADSHAVALTARSLKGRFIIHTMINAYWEGLEFDVPPVSEFECEVWMRWIDTARESPDDISSWDEASVVREAVYPVQPRSLVVLVSRVKNRGKL

SEQ ID NO: 273 - protein, 710 aa, synthetic construct
MDPVTFTTGNAVIDRTRGNSFPLGATVLQGGVNFSVFSKSATFVELLLFDHAEDPGPSRTILLDPRKNRTYHYWHVFVPDVGPGQIYGYRVHGPYEPARGMRFDPGKVLLDPYGRAVVVPEKYSRTAAGLPGDNCGAAMKSVVTDPRTYDWEGDLPLRRPFTRTIIYEMHVRGFTAHPNSGVAPEKRGTYAGLIEKIPYLKDLGITAVELLPVYHFDEQDAPPEFKNYWGYAPVSFFTPHPAYSSRKEPLCVVDEFRDMIKALHRAGIEVILDVVYNHTAEDNHEGPTLSFRGFENDAYYILGPDKRHYSNYTGCGNTLNASNPFVRRMIIDSLHYWVQEMHVDGFRFDLASILARDEQGRPLANPPVLWDIETDPVLSGVKLIAEAWDAAGLYQVGSFIGDSWKEWNVRFRDDVRSFLKGDRNTVSKFASRLLGSPDIYGHEEREPEQSINFVTCHDGFTLNDLVSYNDKHNEENGEENRDGSNDNMSWNCGIEGPTDALPIERLRNRQVKNFFAVTLLAAGAPMLLMGDEVRRTQRGNNNAYGQDNEISWFDWGLVEKHANVLRFVKYLITARLRRNVSLEDLGLTLNQLLSQAKITWHGVKLNQPDWGADSHAVALTARSLKGRFIIHTMINAYWEGLEFDVPPVSEFECEVWMRWIDTARESPDDISSWDEASVVREAVYPVQPRSLVVLVSRVKNRGKLHHHHHH

SEQ ID NO: 274 - protein, 872 aa, synthetic construct
MKKFLFILLLIIAMLGCVQSGIFNNAGNNKGNTISDAKYGTLVIKNIDNGKSTNIDPTRSVVISDIKTAMVTVSGRGMNDISESAAVVNGAGEIRVENIPIGKNRVISVQARTDSGDMTGIVMRGIKDIVSGDNTVNIRWSSTPLGDTFYELLYTHDYDIGSMSDATRSNITAIISGTGVSHPSLFNSKNLAEDVANGTMKSANDASYKITPASAVFSINGITNFTGFTAQVNDPASAKLSSSTTPITTGENTITGIKPGNWELVITIDGGIVYRSGVVALSSGMPYNFGAIDLLVSDPYFLPVNTTFAASLDVTIDVSTTGASIYYTTNGVDPTESDTKYTAPIKITGTTTFKARAFKTNYLSSNVITKTYTKIISSAIGENHPSTGAFSPMDLNGETAGFGWATQTWDLGSHFAGTDVTFAVYSKNATKVLLEIYETETSSTNTNKAYGTDARYDYWMEKGSDNIWRAKIAAVPEKTYYAFRVWGPNWTFNSSWTRGNSSAGFLADVDANGNRFNPNKVVFDPYTREISHDKEFPAMAAAGENGGMYGTSGAEIDAEHIYSGPTTTGGVSINRRNVDTGRWVPKGIVLKPTGKTFTKPTFAQEASVIYEAHVRGITAHSSSSNLETILNGMEGFATVVNVPSQYRGTYKGVAYLAKYLKAIGINTIELLPVHETENDMLPFDGSQPTLGGAKNFWGYMTYGFFAPDRRYSYDKSAGGPTSEFQDMVQALNAEGIKVFLDVVFNHTGEGGNWGHMNVTGFVSMGGLDCAEYYHLIPGGDAKNWLVDGATGCGNQLNFSKTVNHNLIMDSLTYWIDKMGISGYRFDLAAVIGRKPDLHAWEPSDTYWDRVKAFYSDHPTLTAMASLGASKSA

SEQ ID NO: 275 - protein, 862 aa, synthetic construct
CVQSGIFNNAGNNKGNTISDAKYGTLVIKNIDNGKSTNIDPTRSVVISDIKTAMVTVSGRGMNDISESAAVVNGAGEIRVENIPIGKNRVISVQARTDSGDMTGIVMRGIKDIVSGDNTVNIRWSSTPLGDTFYELLYTHDYDIGSMSDATRSNITAIISGTGVSHPSLFNSKNLAEDVANGTMKSANDASYKITPASAVFSINGITNFTGFTAQVNDPASAKLSSSTTPITTGENTITGIKPGNWELVITIDGGIVYRSGVVALSSGMPYNFGAIDLLVSDPYFLPVNTTFAASLDVTIDVSTTGASIYYTTNGVDPTESDTKYTAPIKITGTTTFKARAFKTNYLSSNVITKTYTKIISSAIGENHPSTGAFSPMDLNGETAGFGWATQTWDLGSHFAGTDVTFAVYSKNATKVLLEIYETETSSTNTNKAYGTDARYDYWMEKGSDNIWRAKIAAVPEKTYYAFRVWGPNWTFNSSWTRGNSSAGFLADVDANGNRFNPNKVVFDPYTREISHDKEFPAMAAAGENGGMYGTSGAEIDAEHIYSGPTTTGGVSINRRNVDTGRWVPKGIVLKPTGKTFTKPTFAQEASVIYEAHVRGITAHSSSSNLETILNGMEGFATVVNVPSQYRGTYKGVAYLAKYLKAIGINTIELLPVHETENDMLPFDGSQPTLGGAKNFWGYMTYGFFAPDRRYSYDKSAGGPTSEFQDMVQALNAEGIKVFLDVVFNHTGEGGNWGHMNVTGFVSMGGLDCAEYYHLIPGGDAKNWLVDGATGCGNQLNFSKTVNHNLIMDSLTYWIDKMGISGYRFDLAAVIGRKPDLHAWEPSDTYWDRVKAFYSDHPTLTAMASLGASKSAHHHHHH

SEQ ID NO: 276 - protein, 1435 aa, synthetic construct
MILKKIKEYSKTFTFAIIIVALVVSFGCSNHSSLKYDNEDQDFAYEKGYGLLEVNIPTVRGWTVAQYDVVATKPGETSVTASTASTSVSLRLKIGTWTISVVGKDSYANTIYQGVASANVTESGNSVTIGLLKKAGNYKLTLNSTYPVVSGQPGYIEKIVVTASRGQGFADVVAESNAFSNSFIFSGLAQGNWTFTARGYAAGLDSDYQPTGTEVLFLEDSYTLNVVASKITSSTQNLNTQKKATPVKFSHLAGTYSGTINVSLSCDTAGATIYYTTDGSNPTTSGTKKTYSTSVAISATTTLKVAAVKSGLTSSIVGSRLYTIDAGVTSTPQMNPTGGTYNSDQNVVLSCADAGAIIYYTTDGSNPTTSSTQYSAPIAVSGNGTTKVIRAIAKAGAKNVSGAASQSYTISYLASSAPTFTPASGNLTTNDNITIECGTSGASIYYTTDGSDPKISGTRTPYTSPFSLAVGTYTVKAYSTASGYADSTVTSGEFNVTQPQQDNSIIFATAETIISGEEWNPSSLAAGINLFGVDIFDTGTFKLEWSETYSGSVTMYENDLTTPLSLTGTGPSRTVSLVSGERYFINFNSTYEMANFSVRVYLGASGGGGALGSNYPSSGSYSPVNMADWGTATWELGANYVSGPPSNLRIGVFAANATKVLLEIYSQKTGQTAQYDYWMAKGSDGVWRAELNSVPNYALYAFRAWGPNWSFDSNWTRGNSASGFTSDVDAAGNRYNPNKVLYDPYAKEISHDKETPEMAAAGENGGMYGTGGLTDLTPHGYRGPCTNNVNIDIRNVDTGKWAPKSVAVYNTTSFGTKPNIAEKDGIIYEAHVRGLTQHSSSISLPSILSGVGINTSSWGTWFTDAERGTYKAAGKMAKYLKALGYTTIELLPVHETANDINPADSPGGNYWGYMTYGYFAPDRRYSYDKTFGGPTKEFKEMCAAFHAEGLEVYLDVVYNHTGEGGNWDPSHIESPVGSGNWIENPDFTTKCKEITSFAGLDNANYYALVSTDKGRYWETTGCGNNMDVSKAVVRDLIKDSLKYWITEMGVDGFRFDLAPVLGRDAAPNYHFNPNGQLLTEIAAMGATYNVEMIAEAWDCQWPGGYQVSNFPAGWGEWNGFYRDSVRKFIKGGGNKTSGYPSFTDAFNGSYGPLDRSSGNTHTGFNDQGGPQKSVNFVVAHDGFTMMDLVSYDSKQNTALSWPFGPSDGGSNDNDSWNTNNDPVLRRQQLRNLWTILMFSRGVPMTVWGDEFARTQNGNNNPYNVDSICTYNNYNMIASDSPNTVSTGYAAAYHNNFGTDTNADNANTIFKFARNVIQLRKNSSALKHSSYDMTYTYKKEDGVSDLQDDNRCVWIRIDGSSVAGGSDYLLLINTYTNLVNYTVPTADAGKKWVRIIDTASWAETNDNYWSAASGATITGSYGVNPYSIVVLQEVAQ

SEQ ID NO: 277 - protein, 1414 aa, synthetic construct
CSNHSSLKYDNEDQDFAYEKGYGLLEVNIPTVRGWTVAQYDVVATKPGETSVTASTASTSVSLRLKIGTWTISVVGKDSYANTIYQGVASANVTESGNSVTIGLLKKAGNYKLTLNSTYPVVSGQPGYIEKIVVTASRGQGFADVVAESNAFSNSFIFSGLAQGNWTFTARGYAAGLDSDYQPTGTEVLFLEDSYTLNVVASKITSSTQNLNTQKKATPVKFSHLAGTYSGTINVSLSCDTAGATIYYTTDGSNPTTSGTKKTYSTSVAISATTTLKVAAVKSGLTSSIVGSRLYTIDAGVTSTPQMNPTGGTYNSDQNVVLSCADAGAIIYYTTDGSNPTTSSTQYSAPIAVSGNGTTKVIRAIAKAGAKNVSGAASQSYTISYLASSAPTFTPASGNLTTNDNITIECGTSGASIYYTTDGSDPKISGTRTPYTSPFSLAVGTYTVKAYSTASGYADSTVTSGEFNVTQPQQDNSIIFATAETIISGEEWNPSSLAAGINLFGVDIFDTGTFKLEWSETYSGSVTMYENDLTTPLSLTGTGPSRTVSLVSGERYFINFNSTYEMANFSVRVYLGASGGGGALGSNYPSSGSYSPVNMADWGTATWELGANYVSGPPSNLRIGVFAANATKVLLEIYSQKTGQTAQYDYWMAKGSDGVWRAELNSVPNYALYAFRAWGPNWSFDSNWTRGNSASGFTSDVDAAGNRYNPNKVLYDPYAKEISHDKETPEMAAAGENGGMYGTGGLTDLTPHGYRGPCTNNVNIDIRNVDTGKWAPKSVAVYNTTSFGTKPNIAEKDGIIYEAHVRGLTQHSSSISLPSILSGVGINTSSWGTWFTDAERGTYKAAGKMAKYLKALGYTTIELLPVHETANDINPADSPGGNYWGYMTYGYFAPDRRYSYDKTFGGPTKEFKEMCAAFHAEGLEVYLDVVYNHTGEGGNWDPSHIESPVGSGNWIENPDFTTKCKEITSFAGLDNANYYALVSTDKGRYWETTGCGNNMDVSKAVVRDLIKDSLKYWITEMGVDGFRFDLAPVLGRDAAPNYHFNPNGQLLTEIAAMGATYNVEMIAEAWDCQWPGGYQVSNFPAGWGEWNGFYRDSVRKFIKGGGNKTSGYPSFTDAFNGSYGPLDRSSGNTHTGFNDQGGPQKSVNFVVAHDGFTMMDLVSYDSKQNTALSWPFGPSDGGSNDNDSWNTNNDPVLRRQQLRNLWTILMFSRGVPMTVWGDEFARTQNGNNNPYNVDSICTYNNYNMIASDSPNTVSTGYAAAYHNNFGTDTNADNANTIFKFARNVIQLRKNSSALKHSSYDMTYTYKKEDGVSDLQDDNRCVWIRIDGSSVAGGSDYLLLINTYTNLVNYTVPTADAGKKWVRIIDTASWAETNDNYWSAASGATITGSYGVNPYSIVVLQEVAQHHHHHH

SEQ ID NO: 278 - protein, 700 aa, synthetic construct
MTETASPTALTTGQPQPLGATREEGGTNFAVYAPDATRVELCLLTEEGERCVDLPERTGNVWHGLLPDDHDYSRGVGQGYGYRVHGHHDPENGVYAQPEVRLLDPYARALSGPEEPIRNEEGHVASLQAPWGLVLDTEVEVPLEDKPQVPWNHTVIYETHVRGLTMTHPGVPEELRGTYAGLACEPVVKYLKDLGITAVELLPVHAHVDDPFLQNKGLHNYWGYSTLSYFAPEPRYSAAARAGRPQDTEQEFRDMVAKLHEAGLEVILDVVYNHTAEGGKGGPLLSWRGLANSTYYWLSQDDLGEYFDFTGTGNSVRMTHRRTVQMVLDSMRHWAALGVDGFRFDLASTLARGELGFDKHSNFLGAVQADPVLNRLKLIAEPWDVGLGGYQVGNFPPPWAEWNDQYRDTVRGFWKGDEGLMSEMGFRLTGSSDIYSANHRHPQASINFITAHDGFTLRDVVSYNDKHNDANGEDNRDGHGNNLSWNMGAEGETDDAEVLRERRKQQRNLLTTLLISQGIPMILGGDELGRTQGGNNNTYAQDNEINWYDWQNADRDLLAFTRRLIGLRQDHPSFRRERFFSGRREEGELPHILWLRYDGQEMNDTDWQNPQTKSVGLFLYGGEEEGEVRDHLLVLLNASHVDLPFNLPSFREQGPEQTCERWTLLLDTADDAAQEQVEADQDTQLTARSIKIFSCSAQQG

SEQ ID NO: 279 - protein, 706 aa, synthetic construct
MTETASPTALTTGQPQPLGATREEGGTNFAVYAPDATRVELCLLTEEGERCVDLPERTGNVWHGLLPDDHDYSRGVGQGYGYRVHGHHDPENGVYAQPEVRLLDPYARALSGPEEPIRNEEGHVASLQAPWGLVLDTEVEVPLEDKPQVPWNHTVIYETHVRGLTMTHPGVPEELRGTYAGLACEPVVKYLKDLGITAVELLPVHAHVDDPFLQNKGLHNYWGYSTLSYFAPEPRYSAAARAGRPQDTEQEFRDMVAKLHEAGLEVILDVVYNHTAEGGKGGPLLSWRGLANSTYYWLSQDDLGEYFDFTGTGNSVRMTHRRTVQMVLDSMRHWAALGVDGFRFDLASTLARGELGFDKHSNFLGAVQADPVLNRLKLIAEPWDVGLGGYQVGNFPPPWAEWNDQYRDTVRGFWKGDEGLMSEMGFRLTGSSDIYSANHRHPQASINFITAHDGFTLRDVVSYNDKHNDANGEDNRDGHGNNLSWNMGAEGETDDAEVLRERRKQQRNLLTTLLISQGIPMILGGDELGRTQGGNNNTYAQDNEINWYDWQNADRDLLAFTRRLIGLRQDHPSFRRERFFSGRREEGELPHILWLRYDGQEMNDTDWQNPQTKSVGLFLYGGEEEGEVRDHLLVLLNASHVDLPFNLPSFREQGPEQTCERWTLLLDTADDAAQEQVEADQDTQLTARSIKIFSCSAQQGHHHHHH

SEQ ID NO: 280 - protein, 688 aa, synthetic construct
MMSNLFDAAHGNVEPLGSSVSDNGVNFSLYAPNASQAYVCLFDKSGHSEILKMAMNINEGGVWSIHISPLSAGALYGFRVEGEYNPSHGLLFNENKLLIDPYAKDIFGEFTWSERHYGQMPIGTKSCVNNSIDIPKSKVAAQVHYEHKKPKHRWSNTVIYECHVKGTTCRHPKIPKSLQGKFLGLSHPSFIEHLHNLGVTAVELLPVHAFISEQFLTAKGLQNYWGYNTLNFFTPHKDYLVNDDINEFKQMVKQFHDADIEVILDVVYNHTAEAGNDGPILSLRGLDNLAYYRTAHDQPNVYINDTGCGNTINIDHPKTLQLVLDSLRYWVEVMGVDGFRFDLATILARSKSGFSSGHTFLQAIAQDPVLNKVKLISEPWDIGPGGYQLGAFPPPWREWNDQYRDVIRRFWQSETGIIANVAKRLHGSFDIFEHSQRGPLNSINFITSHDGFTLADLVSYEHKHNEANGEQNRDGHSANHSFNCGVEGFTSDVKITSLRLQQQKNFLLTLILSKGVPMIAAGSEMAHSQGGNNNAYCQNNRTSWLAWKDSQLNHSLTRFIDDALKIRRAHSAFKHSVFLDDIDERFTVKWFTEQGKTMTDAHWHEQTRQFLMYSLLDKQNKHALLIVFNASKKTIFCQLPPSPIKAQWQMVLSSVNNASARSNEDAMVEISAQSSWVFSANLEDVGHG

SEQ ID NO: 281 - protein, 694 aa, synthetic construct
MMSNLFDAAHGNVEPLGSSVSDNGVNFSLYAPNASQAYVCLFDKSGHSEILKMAMNINEGGVWSIHISPLSAGALYGFRVEGEYNPSHGLLFNENKLLIDPYAKDIFGEFTWSERHYGQMPIGTKSCVNNSIDIPKSKVAAQVHYEHKKPKHRWSNTVIYECHVKGTTCRHPKIPKSLQGKFLGLSHPSFIEHLHNLGVTAVELLPVHAFISEQFLTAKGLQNYWGYNTLNFFTPHKDYLVNDDINEFKQMVKQFHDADIEVILDVVYNHTAEAGNDGPILSLRGLDNLAYYRTAHDQPNVYINDTGCGNTINIDHPKTLQLVLDSLRYWVEVMGVDGFRFDLATILARSKSGFSSGHTFLQAIAQDPVLNKVKLISEPWDIGPGGYQLGAFPPPWREWNDQYRDVIRRFWQSETGIIANVAKRLHGSFDIFEHSQRGPLNSINFITSHDGFTLADLVSYEHKHNEANGEQNRDGHSANHSFNCGVEGFTSDVKITSLRLQQQKNFLLTLILSKGVPMIAAGSEMAHSQGGNNNAYCQNNRTSWLAWKDSQLNHSLTRFIDDALKIRRAHSAFKHSVFLDDIDERFTVKWFTEQGKTMTDAHWHEQTRQFLMYSLLDKQNKHALLIVFNASKKTIFCQLPPSPIKAQWQMVLSSVNNASARSNEDAMVEISAQSSWVFSANLEDVGHGHHHHHH

SEQ ID NO: 282 - protein, 676 aa, synthetic construct
MPLAPLVRSGSPLPLGANVTDEGVNFAVASEDATQIYLCLFNENDEEEHVIPFVSHHRGIWHMEVLGLKEGQHYGFRAEGTFSPKDQLMFNRHKLLIDPYAKSLTGTLEWHPDLSAMYSDGHFNPIDTAHYVPKSIVKTITRPDDAPARVSVKGAERSLYELHIKGFSKNLEVSPELKGTYLGVVSEQGISHLKELGVTTIQLMPCFAFANERHLQKLGLQNYWGYNPINFFTPDHRYAVEDPVIECQTMIMSLKRAGFEVIIDVVYNHTAESELDQASFSFKGLDNARYYRHENGEYLNYTGCGNCVNTYHPAAVRLICDSMRYWVDVMGVDGFRFDLGVDLGREKHDFSSTSPLLQAILQDPVLSETCLVMEPWDIGPNGYQVGNFPRGFLECNDKYRDIVRRFWRGDEGIVGEFATRFMGSRDVFHKGYKCALHSVNYISYHDGYTLHDLVSYHRRHNHANMENNRDGHGDNISQNFGVEGPTSDNRIKAQRLNQQLCFLATLLLSQGTPHILGGDELSHTQHGNNNAYCQDNQTTWLGWQNSDEREQLQSAIKTLLNLRKAYPVLAEVFLEDDPLYQYKESDKVEWVNEAGRPMSNEDWSESERDFLSVLFTTSDLSSRLWLIFYREDQVLDVPLIKNARQHQLLFYTPGVAMRDNHLHITERSVALVKLLP

SEQ ID NO: 283 - protein, 682 aa, synthetic construct
MPLAPLVRSGSPLPLGANVTDEGVNFAVASEDATQIYLCLFNENDEEEHVIPFVSHHRGIWHMEVLGLKEGQHYGFRAEGTFSPKDQLMFNRHKLLIDPYAKSLTGTLEWHPDLSAMYSDGHFNPIDTAHYVPKSIVKTITRPDDAPARVSVKGAERSLYELHIKGFSKNLEVSPELKGTYLGVVSEQGISHLKELGVTTIQLMPCFAFANERHLQKLGLQNYWGYNPINFFTPDHRYAVEDPVIECQTMIMSLKRAGFEVIIDVVYNHTAESELDQASFSFKGLDNARYYRHENGEYLNYTGCGNCVNTYHPAAVRLICDSMRYWVDVMGVDGFRFDLGVDLGREKHDFSSTSPLLQAILQDPVLSETCLVMEPWDIGPNGYQVGNFPRGFLECNDKYRDIVRRFWRGDEGIVGEFATRFMGSRDVFHKGYKCALHSVNYISYHDGYTLHDLVSYHRRHNHANMENNRDGHGDNISQNFGVEGPTSDNRIKAQRLNQQLCFLATLLLSQGTPHILGGDELSHTQHGNNNAYCQDNQTTWLGWQNSDEREQLQSAIKTLLNLRKAYPVLAEVFLEDDPLYQYKESDKVEWVNEAGRPMSNEDWSESERDFLSVLFTTSDLSSRLWLIFYREDQVLDVPLIKNARQHQLLFYTPGVAMRDNHLHITERSVALVKLLPHHHHHH

SEQ ID NO: 284 - protein, 759 aa, synthetic construct
MDARPKVTPLTRPTARRSHVSPLGVHVTPDGGVDVAVVASHATGVDLCLIDVVDPSRDERDPARYRERRVPLDGPTYGVWHAHVPGVAPGQRYGFRAHGPWDPAAGHRHNPAKLLVDPYARGLVGEVAYGPETLGSPGRPRSGDPSTPDGRWWLADPYGEPDEVDSIAHVPHAVVMPALPGPAPLSRPRVPWSDTVIYEAHVRGLTQLNPDVPEELRGTYAGVAHPATIAHLQALGVTTLELLPIHASVSEPRLVAQGLTNYWGYQTLGFFAPHARYATKGAQEAGPAAVLDEVRGMVHLLHEAGIEVILDVVYNHTCEGGEDSLHLSWRGLDNAMYYVHDGGTPARLADVTGTGNSLDFRRVRVVQMALDSLRYWAEVVGVDGFRFDLAVTLARGHYGFDPDHPFLVGLQTDPVLSGLKLVAEPWDVGPRGWRTGQFPPPVAEWNDKFRDSVRAFWLDAPRQGSRGQPMHGMRELATRLAGSADLFGHSDPPLVRGPVASINFVTAHDGFTLADLVAFDHKHNWANGEDNRDGHGHNLSWNHGVEGHAPEGDDASTEPWSTVVPLRRRSQRNLLAMTLLAAGTPMLTAGDEIGRSQRGNNNAYALDDETSWLGWDVDDAARHLLETARFLVGLRRRHAALRAESFFLGKPRPGETLPDLLWFDAGGSPMDHDAWNVAGRRVIQMLRTGPGAGESDVLLVLNGGLDDVEVHLPQPLDGAGAWERVWSSTWDTPEEPEEEPLTCPVTLEALSVEVFVAPR

SEQ ID NO: 285 - protein, 765 aa, synthetic construct
MDARPKVTPLTRPTARRSHVSPLGVHVTPDGGVDVAVVASHATGVDLCLIDVVDPSRDERDPARYRERRVPLDGPTYGVWHAHVPGVAPGQRYGFRAHGPWDPAAGHRHNPAKLLVDPYARGLVGEVAYGPETLGSPGRPRSGDPSTPDGRWWLADPYGEPDEVDSIAHVPHAVVMPALPGPAPLSRPRVPWSDTVIYEAHVRGLTQLNPDVPEELRGTYAGVAHPATIAHLQALGVTTLELLPIHASVSEPRLVAQGLTNYWGYQTLGFFAPHARYATKGAQEAGPAAVLDEVRGMVHLLHEAGIEVILDVVYNHTCEGGEDSLHLSWRGLDNAMYYVHDGGTPARLADVTGTGNSLDFRRVRVVQMALDSLRYWAEVVGVDGFRFDLAVTLARGHYGFDPDHPFLVGLQTDPVLSGLKLVAEPWDVGPRGWRTGQFPPPVAEWNDKFRDSVRAFWLDAPRQGSRGQPMHGMRELATRLAGSADLFGHSDPPLVRGPVASINFVTAHDGFTLADLVAFDHKHNWANGEDNRDGHGHNLSWNHGVEGHAPEGDDASTEPWSTVVPLRRRSQRNLLAMTLLAAGTPMLTAGDEIGRSQRGNNNAYALDDETSWLGWDVDDAARHLLETARFLVGLRRRHAALRAESFFLGKPRPGETLPDLLWFDAGGSPMDHDAWNVAGRRVIQMLRTGPGAGESDVLLVLNGGLDDVEVHLPQPLDGAGAWERVWSSTWDTPEEPEEEPLTCPVTLEALSVEVFVAPRHHHHHH

SEQ ID NO: 286 - protein, 698 aa, synthetic construct
MNLTFSELDFMKPELGAEYTGQGTHFAVFSAHAEKIELCLFSEDGREETARMPLPKREGDIWSGYIEGIGPGTLYGYRAHGPYDPHNGHRFNPNKLLLDPYAKLVAGDLIWDDALFGYTIGSPEGDLSFDERDSAPFMVKGVVQDPTFDWAGEEAIRRPWTETIIYESHVRGMTMTHPGVPDELRGTFLGMASDPIIEHLTKLGVSAIELMPVQYFLDDRYLVERNLRNYWGYQTLGFFAPHDRYLKGDRFTEFKTMVKRFHSVGIEVLMDVVYNHTAEGSERGPTLSFRGLDNFSYYRQSPDKPRHTYDTTGTGNTLNVAHPLVLRMVLDSLRYWVGVMHIDGFRFDLASTLGREYMEFDREGGFFDAIRQDPILAGVKLIAEPWDVGEGGYQLGGFPPPFREWNDRFRDDVRRFWKGDGGMVPVLAERILGSPVQFRHSDRAATSSINLVSAHDGFTLMDTVSYGEKHNEANGEENRDGHSDNHSDNMGAEGPTDNREIIEARKRRRAAMLATLMISQGVPMVLGGDELGNSQQGNNNAYCQDNEIGWLDWSGADDGFVDFCRKLLAFRKDRPVLRQERFLDGEPDENGRVEIAWYKADGSPMDEDAWHDAELRLICAYICRAGQGEAPPAGEIVLVLNAGGDCEIALPHGNGSRRWLRVLDTAADEPFGMRASADRDTIPAQSVVAFVPQEGSNG

SEQ ID NO: 287 - protein, 704 aa, synthetic construct
MNLTFSELDFMKPELGAEYTGQGTHFAVFSAHAEKIELCLFSEDGREETARMPLPKREGDIWSGYIEGIGPGTLYGYRAHGPYDPHNGHRFNPNKLLLDPYAKLVAGDLIWDDALFGYTIGSPEGDLSFDERDSAPFMVKGVVQDPTFDWAGEEAIRRPWTETIIYESHVRGMTMTHPGVPDELRGTFLGMASDPIIEHLTKLGVSAIELMPVQYFLDDRYLVERNLRNYWGYQTLGFFAPHDRYLKGDRFTEFKTMVKRFHSVGIEVLMDVVYNHTAEGSERGPTLSFRGLDNFSYYRQSPDKPRHTYDTTGTGNTLNVAHPLVLRMVLDSLRYWVGVMHIDGFRFDLASTLGREYMEFDREGGFFDAIRQDPILAGVKLIAEPWDVGEGGYQLGGFPPPFREWNDRFRDDVRRFWKGDGGMVPVLAERILGSPVQFRHSDRAATSSINLVSAHDGFTLMDTVSYGEKHNEANGEENRDGHSDNHSDNMGAEGPTDNREIIEARKRRRAAMLATLMISQGVPMVLGGDELGNSQQGNNNAYCQDNEIGWLDWSGADDGFVDFCRKLLAFRKDRPVLRQERFLDGEPDENGRVEIAWYKADGSPMDEDAWHDAELRLICAYICRAGQGEAPPAGEIVLVLNAGGDCEIALPHGNGSRRWLRVLDTAADEPFGMRASADRDTIPAQSVVAFVPQEGSNGHHHHHH

SEQ ID NO: 288 - protein, 715 aa, synthetic construct
MSPHIDSHPGKMSSLPPSLRLPGSPFPLGAKVRGTGTQFAVHAPDAELVQVCLISGDGDEMRIDLVQRTYGIWHGIVEGVGAGQRYGYRTHGRWDPARGVRLNPAKLLLDPWATRITGKLGDHDALVSHDGDPFGGPSRVDSLGHTPLSVVTADHARTSGPRIERPWEETVVYELHVGSFTARHPDVPPQYRGTYLGLAEPAVVGHLVRLGVTAVELLPVQAYVSEATVRSRGMRNHWGYSTAAFFAPHPGYAVVPGEEITEFHTMVDALHAAGIEVILDVVYNHTCEQGVDGISLSWRGLDAPGYYLLDSDGRDIDITGCGNTVDAQSPVVVRMIGDSLRYWAHEMGVDGFRFDLASALGRPGGGRFDSRASIFSAITADPVLSRCKLIAEPWDATSDGYRVGHFGVQWSEWNDKYRNTVRRFWNGGTGVRDLASRVAGSEDLFGNRRPWASVNFVTAHDGFTAADLVSYVRKHNEANGEDNRDGADHNDSVDHGVEGPTDDPSIIETRSRHVRALLATLALSTGTPMFTAGDEFGHTLGGNNNAYCVPEDTPREDSWPLDWSTGDTTLTAFVTRALDLRRASPALRQPEFFEGRRTPTGHPDLVWFGADGAEMDDPAWYDDSRRTLQAWIDGSDIRSHTEDGRQLTDDSWLLVFHSGGPAVITLGCPEWFYGTVLAEFDSTSPDGAPAPGGPISGDSTIALSGPTLLVFRAVG

SEQ ID NO: 289 - protein, 721 aa, synthetic construct
MSPHIDSHPGKMSSLPPSLRLPGSPFPLGAKVRGTGTQFAVHAPDAELVQVCLISGDGDEMRIDLVQRTYGIWHGIVEGVGAGQRYGYRTHGRWDPARGVRLNPAKLLLDPWATRITGKLGDHDALVSHDGDPFGGPSRVDSLGHTPLSVVTADHARTSGPRIERPWEETVVYELHVGSFTARHPDVPPQYRGTYLGLAEPAVVGHLVRLGVTAVELLPVQAYVSEATVRSRGMRNHWGYSTAAFFAPHPGYAVVPGEEITEFHTMVDALHAAGIEVILDVVYNHTCEQGVDGISLSWRGLDAPGYYLLDSDGRDIDITGCGNTVDAQSPVVVRMIGDSLRYWAHEMGVDGFRFDLASALGRPGGGRFDSRASIFSAITADPVLSRCKLIAEPWDATSDGYRVGHFGVQWSEWNDKYRNTVRRFWNGGTGVRDLASRVAGSEDLFGNRRPWASVNFVTAHDGFTAADLVSYVRKHNEANGEDNRDGADHNDSVDHGVEGPTDDPSIIETRSRHVRALLATLALSTGTPMFTAGDEFGHTLGGNNNAYCVPEDTPREDSWPLDWSTGDTTLTAFVTRALDLRRASPALRQPEFFEGRRTPTGHPDLVWFGADGAEMDDPAWYDDSRRTLQAWIDGSDIRSHTEDGRQLTDDSWLLVFHSGGPAVITLGCPEWFYGTVLAEFDSTSPDGAPAPGGPISGDSTIALSGPTLLVFRAVGHHHHHH

SEQ ID NO: 290 - protein, 727 aa, synthetic construct
MSHSAQPVTSVQAVWPGRPYPLGATWDGLGVNFALYSQHAEAVELVLFDHPDDPAPSRTIEVTERTGPIWHVYLPGLRPGQLYGYRVYGPYRPEESHRFNPNKVLLDPYAKAIGRPLRWHDSLFGYKIGDPAGDLSFSEEDSAPYAPLGAVVEGCFEWGDDRPPRIPWEDTIIYETHVKGITKLHPEVPEPLRGTYLGLTCEPVLEHLKRLGVTTIQLLPVHAKVHDRHLVERGLRNYWGYNPLCYFAPEPEYATNGPISAVREFKMMVRALHAAGFEVIVDVVYNHTGEGGVLGPTLSFRGIDNRAYYKADPNNPRFLVDYTGTGNTLDVGNPYVIQLIMDSLRYWVTEMHVDGFRFDLAAALARELYDVDMLSTFFQVIQQDPVLSQVKLIAEPWDVGPGGYQVGHFPWQWTEWNGRYRDAVRRFWRGDRGLNGEFATRFAGSSDLYERSGRRPFASINFVTAHDGFTLEDLVSYTKKHNEANLEGNRDGMDENYSTNCGVEGPTQDPSVLACREALKRSLISTLFLSQGVPMLLGGDELSRTQHGNNNAYCQDNEISWYNWQLDTRKQQFLEFVRQVIWFRKQHRSFRRRHFLTGLPNGGEAPDAVWWHPEGRPMRHEDWTNPELTAFGLLLHGDAIQGTDEHGRPFRDDTFLILFNNGSEAVPVVVPEVCSCGKPHHWEVVPVFQRNVEPPTCAPGETLSLPPGVLTVLVAVPPFSDGNPETA

SEQ ID NO: 291 - protein, 733 aa, synthetic construct
MSHSAQPVTSVQAVWPGRPYPLGATWDGLGVNFALYSQHAEAVELVLFDHPDDPAPSRTIEVTERTGPIWHVYLPGLRPGQLYGYRVYGPYRPEESHRFNPNKVLLDPYAKAIGRPLRWHDSLFGYKIGDPAGDLSFSEEDSAPYAPLGAVVEGCFEWGDDRPPRIPWEDTIIYETHVKGITKLHPEVPEPLRGTYLGLTCEPVLEHLKRLGVTTIQLLPVHAKVHDRHLVERGLRNYWGYNPLCYFAPEPEYATNGPISAVREFKMMVRALHAAGFEVIVDVVYNHTGEGGVLGPTLSFRGIDNRAYYKADPNNPRFLVDYTGTGNTLDVGNPYVIQLIMDSLRYWVTEMHVDGFRFDLAAALARELYDVDMLSTFFQVIQQDPVLSQVKLIAEPWDVGPGGYQVGHFPWQWTEWNGRYRDAVRRFWRGDRGLNGEFATRFAGSSDLYERSGRRPFASINFVTAHDGFTLEDLVSYTKKHNEANLEGNRDGMDENYSTNCGVEGPTQDPSVLACREALKRSLISTLFLSQGVPMLLGGDELSRTQHGNNNAYCQDNEISWYNWQLDTRKQQFLEFVRQVIWFRKQHRSFRRRHFLTGLPNGGEAPDAVWWHPEGRPMRHEDWTNPELTAFGLLLHGDAIQGTDEHGRPFRDDTFLILFNNGSEAVPVVVPEVCSCGKPHHWEVVPVFQRNVEPPTCAPGETLSLPPGVLTVLVAVPPFSDGNPETAHHHHHH

SEQ ID NO: 292 - protein, 697 aa, synthetic construct
MIVIEEGLPTPLGAHFDGRGVNFALFSQNATRVDLCLFDPGARHESRTIRLPCRTDDVFHGYLHGLLPGQQYAYRVFGPWDPAAGHRFNPAKLVLDPYAREIAGRIRWHDALYSHRHGGAREDQIDRRDSAPFVPRGVVTRPDTPDAIALPAPRPLHETVIYEAHVKALTRTHPALSEAERGTYLGLAHPAIIEHLLKLGVTALELLPIQAFADDRFLIDKGLVNFWGYQPYNYFAPEPRYLGEGGASGLRFAIRELASAGIETLIDVVYNHTAEGDHRGPTLAFRGIDNASYYKLDPDNLRRNIDCTGCGNTLNVAHPRVMRMVLDSLRHWVTAYGVAGFRFDLATSLGRAPSDFSPQAAFFQAVQQDPVLSRVKLIAEPWDIGAGGYQLGGYPYGWSEWNDQFRDNLRGFWRGDSGTLAKLTQGLSGSREIFLPSGRSPLASINFVASHDGYTLADVVAYEEKHNEANGEENRDGHGHNLSANYGVEGPTDDPALLALRARQKRNMLACVFFAQGVPMLLMGDERSRTQSGNNNAYCQDGALSWMDWENDPDPTLTEFVANLAALRRVCCSLRRRHFLVGSRVGETALKDVHWLSPDGTEMDAAAWGDGERRAFGMQMSNDIEDSERVLILMNAAPEPCPFALPPDLGGPWRPVFDTTLDTGKVLDGARPPVPVGGTVDLPERAVLVLKSPPLLD

SEQ ID NO: 293 - protein, 703 aa, synthetic construct
MIVIEEGLPTPLGAHFDGRGVNFALFSQNATRVDLCLFDPGARHESRTIRLPCRTDDVFHGYLHGLLPGQQYAYRVFGPWDPAAGHRFNPAKLVLDPYAREIAGRIRWHDALYSHRHGGAREDQIDRRDSAPFVPRGVVTRPDTPDAIALPAPRPLHETVIYEAHVKALTRTHPALSEAERGTYLGLAHPAIIEHLLKLGVTALELLPIQAFADDRFLIDKGLVNFWGYQPYNYFAPEPRYLGEGGASGLRFAIRELASAGIETLIDVVYNHTAEGDHRGPTLAFRGIDNASYYKLDPDNLRRNIDCTGCGNTLNVAHPRVMRMVLDSLRHWVTAYGVAGFRFDLATSLGRAPSDFSPQAAFFQAVQQDPVLSRVKLIAEPWDIGAGGYQLGGYPYGWSEWNDQFRDNLRGFWRGDSGTLAKLTQGLSGSREIFLPSGRSPLASINFVASHDGYTLADVVAYEEKHNEANGEENRDGHGHNLSANYGVEGPTDDPALLALRARQKRNMLACVFFAQGVPMLLMGDERSRTQSGNNNAYCQDGALSWMDWENDPDPTLTEFVANLAALRRVCCSLRRRHFLVGSRVGETALKDVHWLSPDGTEMDAAAWGDGERRAFGMQMSNDIEDSERVLILMNAAPEPCPFALPPDLGGPWRPVFDTTLDTGKVLDGARPPVPVGGTVDLPERAVLVLKSPPLLDHHHHHH

SEQ ID NO: 294 - protein, 666 aa, synthetic construct
MMRRLVLEPSGPGPAGAWVGDGGVHWRVFSDNASAVEVCLFDAEGREERLPLPGRDGSFFHGFLPGVGAGLRYGLRAHGPYDPARGHRFNPHKLLIDPLARHLDGPVIPHPALFGFDVARGWPQGEDDQPSGLDSAPFVPKAIVVDPAAPSPRPRLGGRLEPGRLVIYEAHVKGMTQQHPEVETVARGTFAGFAAPGVVDHLRRLGVTAVELLPVWAFADEPALWGSGRTNYWGYNPIAFAAPHPAYGPPDAFCRMVEALHDAGLAVVLDVVINHTAEGGARGPTLSWRGLDNLSWYRLVEGNLAAYVDHTACGNSVQTEHPGVLAQIVNALRAWVVERGVDGFRFDLAVTPARNQGAFDPEGPFLRALAADPVLSSCLLIAEPWDAGPAGHALGRFPAPWLEWNDQARDAVRRFWLRGGSAGAFATALAGSSPVFPAERGPLAGVTYVTCHDGFALADLVAYDTKHNLANGEDNRDGMDTNHSVNHGVEGPTGEPGILALREQQRRNLILSLVLAQGVPMLRAGDELGQTHHGNNNPYCHDSPLTWLDWAATDPTFLAFVRQALALRQPGRCAFLTGQPGPDGWADVAWRRPDGHPMAADDWDDRAFALRLGAARLILVNGRATACSFVLPDDAAWSVVLASAPVDLPALPPLSVAVLAPDLAGE

SEQ ID NO: 295 - protein, 672 aa, synthetic construct
MMRRLVLEPSGPGPAGAWVGDGGVHWRVFSDNASAVEVCLFDAEGREERLPLPGRDGSFFHGFLPGVGAGLRYGLRAHGPYDPARGHRFNPHKLLIDPLARHLDGPVIPHPALFGFDVARGWPQGEDDQPSGLDSAPFVPKAIVVDPAAPSPRPRLGGRLEPGRLVIYEAHVKGMTQQHPEVETVARGTFAGFAAPGVVDHLRRLGVTAVELLPVWAFADEPALWGSGRTNYWGYNPIAFAAPHPAYGPPDAFCRMVEALHDAGLAVVLDVVINHTAEGGARGPTLSWRGLDNLSWYRLVEGNLAAYVDHTACGNSVQTEHPGVLAQIVNALRAWVVERGVDGFRFDLAVTPARNQGAFDPEGPFLRALAADPVLSSCLLIAEPWDAGPAGHALGRFPAPWLEWNDQARDAVRRFWLRGGSAGAFATALAGSSPVFPAERGPLAGVTYVTCHDGFALADLVAYDTKHNLANGEDNRDGMDTNHSVNHGVEGPTGEPGILALREQQRRNLILSLVLAQGVPMLRAGDELGQTHHGNNNPYCHDSPLTWLDWAATDPTFLAFVRQALALRQPGRCAFLTGQPGPDGWADVAWRRPDGHPMAADDWDDRAFALRLGAARLILVNGRATACSFVLPDDAAWSVVLASAPVDLPALPPLSVAVLAPDLAGEHHHHHH

SEQ ID NO: 296 - protein, 662 aa, synthetic construct
MPTTGIPPLGVTRTPDGTRFTVWSHNAARIDLCLFDEAGNTELSRLPMQRDGDVHSIALPDIAAGTRYGLRAEGVYSPDHGLWFDPSKLLVDPYAAQLDRPFRHDQRLTVFGEETADLVPKAILTEVKPLEPRPPLFQAGGLIYEIAVKPFTILHPGVPERKRGTVAALAEPVIIDHLTSLGVSAVELMPVVAWIDERHLPPLGLHNGWGYNPIAPMALDPRLVPGGIEELRRTVEVLHQAGIGVVLDLVFNHSGESDRFGATLSMRGLDNLTYYRHAAGRPGELINDTGCGNTIACDHPVVQRLILDSLRHFVLAAGVDGFRFDLASILGRDMGGFRRDAALFSAISADPILCGRVLIAEPWDTGPGGYQLGNFPHSFLEWNDRARDDIRRYWRGDRHAIGVLATALAGSSDSFSRWGETATRSVNFIAAHDGFTLMDLVSYARKHNEANGEGNRDGHDENFSWNNGAEGATGDPEIAAFRQRDVMALLGTLFTSRGAIMLTAGDEGGRSQGGNNNAYAQDNAVTWLDWSKLDGKLVEHTARLSAMRRRFSVFGDTGFFTGNGDVAWLRLDGEPLTVEDWEHPATDNLVVMLATEDRRQKRPTRLAVVINRSHAPHPFRLPLSLEGEWRDALSDLTVPSFAPARSVTFLVEVFESISRKRI

SEQ ID NO: 297 - protein, 668 aa, synthetic construct
MPTTGIPPLGVTRTPDGTRFTVWSHNAARIDLCLFDEAGNTELSRLPMQRDGDVHSIALPDIAAGTRYGLRAEGVYSPDHGLWFDPSKLLVDPYAAQLDRPFRHDQRLTVFGEETADLVPKAILTEVKPLEPRPPLFQAGGLIYEIAVKPFTILHPGVPERKRGTVAALAEPVIIDHLTSLGVSAVELMPVVAWIDERHLPPLGLHNGWGYNPIAPMALDPRLVPGGIEELRRTVEVLHQAGIGVVLDLVFNHSGESDRFGATLSMRGLDNLTYYRHAAGRPGELINDTGCGNTIACDHPVVQRLILDSLRHFVLAAGVDGFRFDLASILGRDMGGFRRDAALFSAISADPILCGRVLIAEPWDTGPGGYQLGNFPHSFLEWNDRARDDIRRYWRGDRHAIGVLATALAGSSDSFSRWGETATRSVNFIAAHDGFTLMDLVSYARKHNEANGEGNRDGHDENFSWNNGAEGATGDPEIAAFRQRDVMALLGTLFTSRGAIMLTAGDEGGRSQGGNNNAYAQDNAVTWLDWSKLDGKLVEHTARLSAMRRRFSVFGDTGFFTGNGDVAWLRLDGEPLTVEDWEHPATDNLVVMLATEDRRQKRPTRLAVVINRSHAPHPFRLPLSLEGEWRDALSDLTVPSFAPARSVTFLVEVFESISRKRIHHHHHH

SEQ ID NO: 298 - protein, 370 aa, synthetic construct
MAATQACGRCEMSGLTVSIRGRNGAFAIEAGFAAEGGVTALFGHSGAGKTTLLKMIAGTLRPENGRIAVGDFTLFDAQKGINLPPEKRRIGYVFQDARLFAYMSVKRNLTYARWAGHRQATRSFDEVVALLGIGHLLDRRPSTLSGGERQRVAIGRALLSDPALLLLDEPLSSLDHARRQEILPFIERLRDESHVPIVYVSHEIDEVARLADQIVLLSAGRVTASGAAADIFPLIDAESEGGGVLLEGIVSAYDERYKLAEIDLGGASFQLSDAGLKQTMHVRLRVRARDVSIARKIPEAISIRNLLPVTVTGIERGEGPNAHVFLDFRGRRLGARLTRRSVDDLGLSVGDQVVALVKAVSVDRAAIREK

SEQ ID NO: 299 - protein, 376 aa, synthetic construct
MAATQACGRCEMSGLTVSIRGRNGAFAIEAGFAAEGGVTALFGHSGAGKTTLLKMIAGTLRPENGRIAVGDFTLFDAQKGINLPPEKRRIGYVFQDARLFAYMSVKRNLTYARWAGHRQATRSFDEVVALLGIGHLLDRRPSTLSGGERQRVAIGRALLSDPALLLLDEPLSSLDHARRQEILPFIERLRDESHVPIVYVSHEIDEVARLADQIVLLSAGRVTASGAAADIFPLIDAESEGGGVLLEGIVSAYDERYKLAEIDLGGASFQLSDAGLKQTMHVRLRVRARDVSIARKIPEAISIRNLLPVTVTGIERGEGPNAHVFLDFRGRRLGARLTRRSVDDLGLSVGDQVVALVKAVSVDRAAIREKHHHHHH

SEQ ID NO: 300 - protein, 718 aa, synthetic construct
MLHISRTFAAYLDEMDQIVVLAPKSLGFDGMAPFTLVAPSGEEIPLSVQHVEDVGETVKYVCRFASAFEFGATYWVRSCRGEETDVQIGAVVRTPAFDDRFFYDGPLGAEYLKEQTVFRVWAPTATAVSVKLVHPHLDEIRCVPLVRGERGVWSAVVPGDWERARYTYIACINRVWREAVDPYATAVSVNGEFGVVIDWEKTKLAPPSLPLPPLCSPTDAIIYELSIRDFTSHPDSGAVHKGKYLGLAETNTSGPNGTATGLSYVKELGVTHVQLMPFMDFAGVDERDPQAAYNWGYNPLHLYAPEGSYATDPADPYARIVELKQAIHTLHENGLRVVMDAVYNHVYDREQSPLEKLVPGYYFRYDAYGQPANGTGVGNDIASERRMARRWIVDSVVFWAKEYGIDGFRFDLMGVHDIETMKAVRDALDAIDPSILVYGEGWDLPTPLPPEQKATMANAKQLPRFAYFNDRFRDAVKGSTFHLPDRGFALGNPGGREQVKLAIAGSLRALGGLFCHPRQSINYVECHDNHTFWDKMEAANHDEPEWLRRKRQKLATAIVLLAQGIPFLHSGQEFYRTKGGDGNSYRSPDAVNQLDWERKSRYEDDVRYVQGLIALRRAHGAFRLATEAEVLRHFTFLEPLPPSVIAYRLHDAAVYGPWEDIIVVHHNEEKETAIALPDEREWAVVCDGQRCGTTPFGQARGMLRLDGIGTWVLVHPAG

SEQ ID NO: 301 - protein, 724 aa, synthetic construct
MLHISRTFAAYLDEMDQIVVLAPKSLGFDGMAPFTLVAPSGEEIPLSVQHVEDVGETVKYVCRFASAFEFGATYWVRSCRGEETDVQIGAVVRTPAFDDRFFYDGPLGAEYLKEQTVFRVWAPTATAVSVKLVHPHLDEIRCVPLVRGERGVWSAVVPGDWERARYTYIACINRVWREAVDPYATAVSVNGEFGVVIDWEKTKLAPPSLPLPPLCSPTDAIIYELSIRDFTSHPDSGAVHKGKYLGLAETNTSGPNGTATGLSYVKELGVTHVQLMPFMDFAGVDERDPQAAYNWGYNPLHLYAPEGSYATDPADPYARIVELKQAIHTLHENGLRVVMDAVYNHVYDREQSPLEKLVPGYYFRYDAYGQPANGTGVGNDIASERRMARRWIVDSVVFWAKEYGIDGFRFDLMGVHDIETMKAVRDALDAIDPSILVYGEGWDLPTPLPPEQKATMANAKQLPRFAYFNDRFRDAVKGSTFHLPDRGFALGNPGGREQVKLAIAGSLRALGGLFCHPRQSINYVECHDNHTFWDKMEAANHDEPEWLRRKRQKLATAIVLLAQGIPFLHSGQEFYRTKGGDGNSYRSPDAVNQLDWERKSRYEDDVRYVQGLIALRRAHGAFRLATEAEVLRHFTFLEPLPPSVIAYRLHDAAVYGPWEDIIVVHHNEEKETAIALPDEREWAVVCDGQRCGTTPFGQARGMLRLDGIGTWVLVHPAGHHHHHH

SEQ ID NO: 302 - protein, 764 aa, synthetic construct
MRRRELRNGVLTVALLAASAAFSWAGEELAAVAAENSPKVSSAPSAASGQEARYGAHVGADGWVDFVVHAPAATAVDLLLYDDPRARTPQRRVAMTPAGDDWKARVRGSRHGPALFYMYQASGPREVSPERPFGALFNPAYVLNDPYAYRTENVNYDAFFATVPFTDMTTPVYAGGGKSVVYDHSGDGFPGHVRIAPEDLIVYELHVQDYTATLEGLPAARRGTYLGLARGGLKTPGGLAAGIDHLVELGVNAVELMPVMEYDEQSGNQSGRLNHWGYMTSNFFAPEARYAASPGRQVVELKQLVRALHQRGIAVFLDVVYNHTAEQSPWVSDGRLAAKCFNLMCLAADRVYRPTSDGRYYLNNTGTGNDVDFSGGKRYSKQLVRDSLAMWHQIYGIDGFRFDLARILAEGSHDAADWVDNDPRYATAHLHAEPWDMGGQWWDFMDSDGWSADNNRWAKWLGHYRDKVRRFSQSSLNDPDAFKQLIEGRGSVSRGAGPAASSRPWRSINLLAVHDGYTLRDCTWFNDSDGSQNCWDSNQDEDLRRQREKLLLGILLTSQGVPVILQGDEFGRSKAGAAAQEGARNSYNYESSTGDLAVNRVNWIDWRLKDGDNSASPTGPAYGKELFEWTRGLITLRKKWSHFRRHDFARYVHAATDDRAGPINDGGFSYVWEGPRSGEPTQLAVVWWGKAGEPDLMVIYNEGWEPFKVDNLSHWSRGNWKILARSWHGPGQDMCRGKWKQCPAAGSAVTVAGRSMAILVSDND

SEQ ID NO: 303 - protein, 745 aa, synthetic construct
GEELAAVAAENSPKVSSAPSAASGQEARYGAHVGADGWVDFVVHAPAATAVDLLLYDDPRARTPQRRVAMTPAGDDWKARVRGSRHGPALFYMYQASGPREVSPERPFGALFNPAYVLNDPYAYRTENVNYDAFFATVPFTDMTTPVYAGGGKSVVYDHSGDGFPGHVRIAPEDLIVYELHVQDYTATLEGLPAARRGTYLGLARGGLKTPGGLAAGIDHLVELGVNAVELMPVMEYDEQSGNQSGRLNHWGYMTSNFFAPEARYAASPGRQVVELKQLVRALHQRGIAVFLDVVYNHTAEQSPWVSDGRLAAKCFNLMCLAADRVYRPTSDGRYYLNNTGTGNDVDFSGGKRYSKQLVRDSLAMWHQIYGIDGFRFDLARILAEGSHDAADWVDNDPRYATAHLHAEPWDMGGQWWDFMDSDGWSADNNRWAKWLGHYRDKVRRFSQSSLNDPDAFKQLIEGRGSVSRGAGPAASSRPWRSINLLAVHDGYTLRDCTWFNDSDGSQNCWDSNQDEDLRRQREKLLLGILLTSQGVPVILQGDEFGRSKAGAAAQEGARNSYNYESSTGDLAVNRVNWIDWRLKDGDNSASPTGPAYGKELFEWTRGLITLRKKWSHFRRHDFARYVHAATDDRAGPINDGGFSYVWEGPRSGEPTQLAVVWWGKAGEPDLMVIYNEGWEPFKVDNLSHWSRGNWKILARSWHGPGQDMCRGKWKQCPAAGSAVTVAGRSMAILVSDNDHHHHHH

SEQ ID NO: 304 - protein, 666 aa, synthetic construct
MESLSVRSTIPLPLGAKKLSADRYRFSLFSSQAQQVTLVLLDPLSEIHEIPLSSTDHRTGAIWHIEIAGISSEWSYAYKLRGTDLSSQKFATDSYIADPYSKNIYSPQLFGSPKQEKDYAFSYLKHEDFDWEGDTPLHLPKENYFIYEMHVRSFTRDPSSQVSHPGTFLGIIEKIDHLKQLGVHAVELLPIFEFDETVHPFKNQDFPHLCNYWGYSSVNFFCPSRRYTYGADPCAPAREFKTLVKALHRAGIEVILDVVFNHTGFEGTSCPLPWIDLESYYMVNDHGDLMNFSGCGNTVNTNTPTTLKWILDALRYWVQEMHVDGFRFDLASVFSRDPQGVPLPLTPILQAISSDSILSETKLIAEPWDAGGLYQLGHFPSISTRWSEWNGCYRDHVKAFLNGDAHQVSSFASRISGSHDIYPNGKPTNSINYICSHDGFTLYDTVAYNDKHNEENGEYNRDGTSANYSYNFGCEGETTDPTICALRERQMKNFFLALFLSQGIPMIQSGDEYGHTAYGNNNHWCLDTKINYFLWDRLAERKELFSFLCQVIALRKAYTELFNTSFLSEDTIVWLNTKGSPREWGADHYLAFELKHLNYSLFVAFYSGNERIEISLPKPRKEHLAYEKIVDSTTGFFSQILSPKLSLEPYSSLVAISRRKTSLESR

SEQ ID NO: 305 - protein, 672 aa, synthetic construct
MESLSVRSTIPLPLGAKKLSADRYRFSLFSSQAQQVTLVLLDPLSEIHEIPLSSTDHRTGAIWHIEIAGISSEWSYAYKLRGTDLSSQKFATDSYIADPYSKNIYSPQLFGSPKQEKDYAFSYLKHEDFDWEGDTPLHLPKENYFIYEMHVRSFTRDPSSQVSHPGTFLGIIEKIDHLKQLGVHAVELLPIFEFDETVHPFKNQDFPHLCNYWGYSSVNFFCPSRRYTYGADPCAPAREFKTLVKALHRAGIEVILDVVFNHTGFEGTSCPLPWIDLESYYMVNDHGDLMNFSGCGNTVNTNTPTTLKWILDALRYWVQEMHVDGFRFDLASVFSRDPQGVPLPLTPILQAISSDSILSETKLIAEPWDAGGLYQLGHFPSISTRWSEWNGCYRDHVKAFLNGDAHQVSSFASRISGSHDIYPNGKPTNSINYICSHDGFTLYDTVAYNDKHNEENGEYNRDGTSANYSYNFGCEGETTDPTICALRERQMKNFFLALFLSQGIPMIQSGDEYGHTAYGNNNHWCLDTKINYFLWDRLAERKELFSFLCQVIALRKAYTELFNTSFLSEDTIVWLNTKGSPREWGADHYLAFELKHLNYSLFVAFYSGNERIEISLPKPRKEHLAYEKIVDSTTGFFSQILSPKLSLEPYSSLVAISRRKTSLESRHHHHHH

SEQ ID NO: 306 - protein, 692 aa, synthetic construct
MAFRIFQAYLDDENIITIELEKSFEAYSIHFTLEDKHSSSPLTIKNINNQEERIIYTVSANEPIDLTQSYKVYDQDRNHTDLQYRHIVKKPIFDEIFDYAGDDLGAQYNPQATDFKLWAPISEKVLLHLKDQVHHLKRLDKGVWHIRIEGDLEGSSYSYLHKINGKWVEVHDPYALSSDVNSGNSYVINLEKIKKPIKRAKTQLNPTEAVIYEMSVRDFSMQKEIGFSCPGKFASLSESPVVHGQKFGLDYLKELGISHVQLMPVYDFGSVDEKHPELVYNWGYDPVQYNVPDGSFASNPRDPYARILELQAAITAFHNADISVIMDVVYNHVYDANSYAFEKIVPGYFFRLNDMGYRTNGTFCGNDVASEKAMVRRYIKQSVKQWVSLYGFDGFRFDLMGILDIQTMQQIADELKTLYPNIYLYGEGWQMDTGLASERLAHQYNAAQLPDYGFFSDHFRDSLKQTIAQGRQIESKTPASQLENVLTANVGLTGEAHFTAPQQAINYVECHDNATVFDYFDIVNPAITLRDRLANSRLALHLVLLAQGVPFIHSGQEFFRTKNLIDNTYNMPDEINKLDWLRSLHYTEDIAFVKQLIAFRRAHPLLHLKTSAAIKQACQVNWLTDSLLEYKIQDRKESMTIVINFGNQEAVYENKNKQRLHLQYPAIDAQKPIAPLADSYNLAGKQLIVLKS

SEQ ID NO: 307 - protein, 698 aa, synthetic construct
MAFRIFQAYLDDENIITIELEKSFEAYSIHFTLEDKHSSSPLTIKNINNQEERIIYTVSANEPIDLTQSYKVYDQDRNHTDLQYRHIVKKPIFDEIFDYAGDDLGAQYNPQATDFKLWAPISEKVLLHLKDQVHHLKRLDKGVWHIRIEGDLEGSSYSYLHKINGKWVEVHDPYALSSDVNSGNSYVINLEKIKKPIKRAKTQLNPTEAVIYEMSVRDFSMQKEIGFSCPGKFASLSESPVVHGQKFGLDYLKELGISHVQLMPVYDFGSVDEKHPELVYNWGYDPVQYNVPDGSFASNPRDPYARILELQAAITAFHNADISVIMDVVYNHVYDANSYAFEKIVPGYFFRLNDMGYRTNGTFCGNDVASEKAMVRRYIKQSVKQWVSLYGFDGFRFDLMGILDIQTMQQIADELKTLYPNIYLYGEGWQMDTGLASERLAHQYNAAQLPDYGFFSDHFRDSLKQTIAQGRQIESKTPASQLENVLTANVGLTGEAHFTAPQQAINYVECHDNATVFDYFDIVNPAITLRDRLANSRLALHLVLLAQGVPFIHSGQEFFRTKNLIDNTYNMPDEINKLDWLRSLHYTEDIAFVKQLIAFRRAHPLLHLKTSAAIKQACQVNWLTDSLLEYKIQDRKESMTIVINFGNQEAVYENKNKQRLHLQYPAIDAQKPIAPLADSYNLAGKQLIVLKSHHHHHH

SEQ ID NO: 308 - protein, 645 aa, synthetic construct
MYYNFNHYINLGANLEKDGCSFAIYAKNVNSLSLNIFHSSEDTVPYEKHILSPSEHKLGDIWSIFLENIKEGTLYNWEINGMAILDPYALAYTGNKTIENKKSIVLARVGTETKHILIPKKNMIIYESHIGLFTKSPSSNTLNGATYSAFEEKIPYLKNLGINVVEFLPIFEWDDFTGNLDRESFFLKNVWGYNPINFFALTKKYSSSKDENSANEINEFKKLIFSLHKNGIEVILDVVYNHTAEGGTGGKVYNFKAMGENIFYTKDRENYFTNFSGCGNTLNCNHKVVKDMIIQSLLYWYLEVGVDGFRFDLAPVLGRDSNNQWARHSLLHELIEHPILSHAKLIAESWDLGGYFVGAMPSGWCEWNGAYRDTVRQFIRGDFGQVPELIKRIFGSVDIFHANKNGYQSSINFICCHDGFTMWDLVSYNLKHNLLNGENNQDGENNNHSYNHGEEGFTENSHIISLRKQQIKNMILILYISQGIPMLLMGDEMGRTQLGNNNAYCQDNPTTWVDWDRKKDFEDVFLFTKNMISLRKSYSIFKKETPLIEGEEVILHGIKLYQPDLSFHSLSIAFQLKDIKSNTDFYIAFNSYTEQLCFELPILENKSWYILTDTSKVDTCNFQETKCQDTHCCVLPKSSVILISK

SEQ ID NO: 309 - protein, 651 aa, synthetic construct
MYYNFNHYINLGANLEKDGCSFAIYAKNVNSLSLNIFHSSEDTVPYEKHILSPSEHKLGDIWSIFLENIKEGTLYNWEINGMAILDPYALAYTGNKTIENKKSIVLARVGTETKHILIPKKNMIIYESHIGLFTKSPSSNTLNGATYSAFEEKIPYLKNLGINVVEFLPIFEWDDFTGNLDRESFFLKNVWGYNPINFFALTKKYSSSKDENSANEINEFKKLIFSLHKNGIEVILDVVYNHTAEGGTGGKVYNFKAMGENIFYTKDRENYFTNFSGCGNTLNCNHKVVKDMIIQSLLYWYLEVGVDGFRFDLAPVLGRDSNNQWARHSLLHELIEHPILSHAKLIAESWDLGGYFVGAMPSGWCEWNGAYRDTVRQFIRGDFGQVPELIKRIFGSVDIFHANKNGYQSSINFICCHDGFTMWDLVSYNLKHNLLNGENNQDGENNNHSYNHGEEGFTENSHIISLRKQQIKNMILILYISQGIPMLLMGDEMGRTQLGNNNAYCQDNPTTWVDWDRKKDFEDVFLFTKNMISLRKSYSIFKKETPLIEGEEVILHGIKLYQPDLSFHSLSIAFQLKDIKSNTDFYIAFNSYTEQLCFELPILENKSWYILTDTSKVDTCNFQETKCQDTHCCVLPKSSVILISKHHHHHH

SEQ ID NO: 310 - protein, 808 aa, synthetic construct
MEPTEPSTSTEALPADIPVWPGSAYPLGATYDGAGTNFALFSEVAEKVELCLIAKDGTETRVPIEEVDGNVWHVYLPTVTPGQRYGYRVHGPYDPAQGLRCDPSKLLLDPYGKAYDGAFDGDASLFSYAMVGAEDAPSEPEHSAGAEEAQSAPEPAARAAEDAGAEAAGDTAGAEAAGNTTADEADDSDGTGSGETGDDGTGDDGTGDDTAAESAPEPAAPANPDFPQLDSLGHTMTTVVINPFFDWASDRHPRRPYYETVIYEAHVKGMTATHPGVPEQLRGTYAGLAHPVIIDHLQSLGVTAIELMPVHQFMHDQVLLDRGLRNYWGYNTFGFLAPHLDYSSASNPSGVVAEFKAMVRAYHEAGIEVILDVVYNHTAEGNHLGPTISFRGIDNAAYYRLVDDDKSYYMDYTGTGNSLNARHPHTLQLIMDSLRYWVTEMHVDGFRFDLASTLARELHDVDRLSAFFDLVQQDPIVSQVKLIAEPWDVGEGGYQVGNFPGLWTEWNGKYRDTVRDYWRGEPATLGEFASRLTGSSDLYEATGRRPSASINFVTAHDGFTLADLVSYNEKHNDANGENNMDGESHNRSWNCGAEGPTDDPEVLALRQRQSRNILATLMLSQGTPMIAHGDEFGRTQQGNNNVYCQDNELSWMDWSLAETNADLLDFTRNVIALRRSHPVFRRRRFFEGRPIRSGEQSRDIAWRTPAGEEMTPEDWDSGFGKSLSVFLNGEGIHEPNQRGERVVDDSFLLCFNAHDEPIDFVTPDGPHAQEWSVALDTDVPDGLREQVVTAGGSVRVQARSLLVLRRTA

SEQ ID NO: 311 - protein, 814 aa, synthetic construct
MEPTEPSTSTEALPADIPVWPGSAYPLGATYDGAGTNFALFSEVAEKVELCLIAKDGTETRVPIEEVDGNVWHVYLPTVTPGQRYGYRVHGPYDPAQGLRCDPSKLLLDPYGKAYDGAFDGDASLFSYAMVGAEDAPSEPEHSAGAEEAQSAPEPAARAAEDAGAEAAGDTAGAEAAGNTTADEADDSDGTGSGETGDDGTGDDGTGDDTAAESAPEPAAPANPDFPQLDSLGHTMTTVVINPFFDWASDRHPRRPYYETVIYEAHVKGMTATHPGVPEQLRGTYAGLAHPVIIDHLQSLGVTAIELMPVHQFMHDQVLLDRGLRNYWGYNTFGFLAPHLDYSSASNPSGVVAEFKAMVRAYHEAGIEVILDVVYNHTAEGNHLGPTISFRGIDNAAYYRLVDDDKSYYMDYTGTGNSLNARHPHTLQLIMDSLRYWVTEMHVDGFRFDLASTLARELHDVDRLSAFFDLVQQDPIVSQVKLIAEPWDVGEGGYQVGNFPGLWTEWNGKYRDTVRDYWRGEPATLGEFASRLTGSSDLYEATGRRPSASINFVTAHDGFTLADLVSYNEKHNDANGENNMDGESHNRSWNCGAEGPTDDPEVLALRQRQSRNILATLMLSQGTPMIAHGDEFGRTQQGNNNVYCQDNELSWMDWSLAETNADLLDFTRNVIALRRSHPVFRRRRFFEGRPIRSGEQSRDIAWRTPAGEEMTPEDWDSGFGKSLSVFLNGEGIHEPNQRGERVVDDSFLLCFNAHDEPIDFVTPDGPHAQEWSVALDTDVPDGLREQVVTAGGSVRVQARSLLVLRRTAHHHHHH

SEQ ID NO: 312 - protein, 691 aa, synthetic construct
MLPGNAAEWGATLTPEGVNFTLAAPNATSVDLCLFDEAGVTELQRLPLPAKTGDVWHGLLPAGRAGQVYGFRVHGPWAPERGHRFNPAKLLLDPYAREVLGRYDGSDLHWAHAPDSARGRQLPDSRDNAATALKARVCAPLPAANPGPVVLPKQRVMYELHVKGFTRLHPGVPETLRGSYAGLAHPAAIAHLKAMGVTTLSLMPVAFRADEERLQRLGLSNYWGYSPIAWSAPESRYWSGTAGSTPRTEFRAMVDAVHAAGIEVILDVVYNHTGETDEFGPLLSLRGIDNGTYYHLEPGDASRYLNWTGCGNCVNLNHPVVLRTVMDSLRAWVTDYGVDGFRFDLAPVLARGTAETDYRFNPHAPLLMAIAQDPTLRNCVMVAEPWDIGPGGYQLGAFPAGWLEWNDRFRDTQRSAWLQHSTHRGALANRLAGSADAFSPLRRAAHSGVNFVTAHDGFNLMDVVSYCHRHNEANGEHNRDGHGHNLSVNHGVEGGSEDEQVVQARARHRRTLLAVTLFSLGTPMLLAGDELGHSQCGNNNAYCQDNATTWLNWEAARHDLSSFVARAIALRRALPALQATGWWRSHTGEAGSGPVAMWALPDGRTVEAGDWEAPQGGPLAVHLQLGSSGAVLLLLNPSASAQAFTLPDGEWHLCLDTGSESVGTEPASTVSPLFTVQADSLVLLSAVQPVF

SEQ ID NO: 313 - protein, 697 aa, synthetic construct
MLPGNAAEWGATLTPEGVNFTLAAPNATSVDLCLFDEAGVTELQRLPLPAKTGDVWHGLLPAGRAGQVYGFRVHGPWAPERGHRFNPAKLLLDPYAREVLGRYDGSDLHWAHAPDSARGRQLPDSRDNAATALKARVCAPLPAANPGPVVLPKQRVMYELHVKGFTRLHPGVPETLRGSYAGLAHPAAIAHLKAMGVTTLSLMPVAFRADEERLQRLGLSNYWGYSPIAWSAPESRYWSGTAGSTPRTEFRAMVDAVHAAGIEVILDVVYNHTGETDEFGPLLSLRGIDNGTYYHLEPGDASRYLNWTGCGNCVNLNHPVVLRTVMDSLRAWVTDYGVDGFRFDLAPVLARGTAETDYRFNPHAPLLMAIAQDPTLRNCVMVAEPWDIGPGGYQLGAFPAGWLEWNDRFRDTQRSAWLQHSTHRGALANRLAGSADAFSPLRRAAHSGVNFVTAHDGFNLMDVVSYCHRHNEANGEHNRDGHGHNLSVNHGVEGGSEDEQVVQARARHRRTLLAVTLFSLGTPMLLAGDELGHSQCGNNNAYCQDNATTWLNWEAARHDLSSFVARAIALRRALPALQATGWWRSHTGEAGSGPVAMWALPDGRTVEAGDWEAPQGGPLAVHLQLGSSGAVLLLLNPSASAQAFTLPDGEWHLCLDTGSESVGTEPASTVSPLFTVQADSLVLLSAVQPVFHHHHHH

SEQ ID NO: 314 - protein, 763 aa, synthetic construct
MGRSRGTTPRCREDRDAPEAHATDRVARRKYTWMTEINRSDAAHVLGAHLSADGCHFGLWAPRAERVELALVDGDGSTQRNVDMTLGGGAWSVFVPDVVAGQRYGFRVHAQWDPDQGLRANPAKLLVDPYARAITAGVDYSGPIFDHVPGSYFEPDTRDSAGSVPLSVVVADSPAPEPIAERRPLEECVIYETHVKGLTQLHPTVPEHLRGRFAGVAYPAVTEHLKSLGVNAVEFLPVHHFISEPFVMGRGLSNYWGYNSLGFFAPHAAYCSVGTEGDQVAEFKEMVTALHRAGIEVILDVVYNHTCEGNHEGPTLSFRGIDHRGYYRLTDDLRNDYDITGTGNSVNTAHADVLAMVVDSMRYWVQEMGVDGFRFDLATELIRDGEHHVDQNHDFKKLIAQDPAFKGVKMIAEPWDLGPYGYQVGNWGPGWSEWNDRFRGYMRDYWRGQVDGVDELATRLSGSADLFDHDDRPPSSSINFFDAHDGFPLRDLVTYNEKHNEANGEDNRDGSDDNRSWNCGVEGETADEVINALRHRQIRNMVATLMLSDGVPMYCAGDEMGRTQQGNNNAYCQDGPINWLRWDQMEEWGDVLDTVRTFTDLRMSTPLLHANDYRYRTEVTDPTGAGLGRYELAWMNGSSGEMGEADWHDGSRRLLGMYVSDASSVAYLSWFYSGDQPIQVQMPPAPWGESFHIVASTCEDGEVPDADLAPGDSFTMPPRTVVTMRVAVMTTAPVPDDQPVTDQEIATGDPHIPDTPQAAAPTS

SEQ ID NO: 315 - protein, 769 aa, synthetic construct
MGRSRGTTPRCREDRDAPEAHATDRVARRKYTWMTEINRSDAAHVLGAHLSADGCHFGLWAPRAERVELALVDGDGSTQRNVDMTLGGGAWSVFVPDVVAGQRYGFRVHAQWDPDQGLRANPAKLLVDPYARAITAGVDYSGPIFDHVPGSYFEPDTRDSAGSVPLSVVVADSPAPEPIAERRPLEECVIYETHVKGLTQLHPTVPEHLRGRFAGVAYPAVTEHLKSLGVNAVEFLPVHHFISEPFVMGRGLSNYWGYNSLGFFAPHAAYCSVGTEGDQVAEFKEMVTALHRAGIEVILDVVYNHTCEGNHEGPTLSFRGIDHRGYYRLTDDLRNDYDITGTGNSVNTAHADVLAMVVDSMRYWVQEMGVDGFRFDLATELIRDGEHHVDQNHDFKKLIAQDPAFKGVKMIAEPWDLGPYGYQVGNWGPGWSEWNDRFRGYMRDYWRGQVDGVDELATRLSGSADLFDHDDRPPSSSINFFDAHDGFPLRDLVTYNEKHNEANGEDNRDGSDDNRSWNCGVEGETADEVINALRHRQIRNMVATLMLSDGVPMYCAGDEMGRTQQGNNNAYCQDGPINWLRWDQMEEWGDVLDTVRTFTDLRMSTPLLHANDYRYRTEVTDPTGAGLGRYELAWMNGSSGEMGEADWHDGSRRLLGMYVSDASSVAYLSWFYSGDQPIQVQMPPAPWGESFHIVASTCEDGEVPDADLAPGDSFTMPPRTVVTMRVAVMTTAPVPDDQPVTDQEIATGDPHIPDTPQAAAPTSHHHHHH

SEQ ID NO: 316 - protein, 733 aa, synthetic construct
MQKPTADDIKLNPDAKHNPSQNEKWALGAGEPFPLGASVEGDGVNFALFSANATAVELCLFDDNGEQEIARIALTEQTQQIWHVFVHGLKAGQLYGYRVYGSYEPLLGHRFNPNKLLLDPYARQLVGEYSDHESNYGYELNHKDEDLSFSKLDNAAHVPKCKVVDIRPLIDIAAKQSIQPVATRFQPNPLERSIIYEMHVKGFTAAHPEIEPNKRATFAGLATQPAIDYLAELGVTSVELLPVQAFFTEPFLLEKKLTNYWGYNSIGFFAPEPSYLSSDDIGEFRAMVDALHGAGIEVILDVVYNHSAEGSRLGPTFSFRGIDNLSYYRLHPNDKRFYINDTGCGNTLNINHPRMLQLVLDSLRYWVEIMGVDGFRFDLAACLGREAYGFDPGSGFFDALLQDPVLCRVKLIAEPWDIGPGGYQLGNFPVAFSEWNDRYRDTMRRFWRGDHSMLPEFARRFHGSGDFFEHGGRPPATSLNFLTSHDGFTLKDLVSYTQRHNLANGEDNRDGHQENFSHHYGIEGETEDSAILALRSRQQRNLLTTLFLSQGVPMLLSGDETGRTQQGNNNAYCQDNELNWFDWSAKGMDTELLAFTQQLIALRKRFPLLCAKRFIHEQLLAESLADTGARLDWFSRQGEQMTKSLWTESMCRSLSVVLSGDLEGQGKQQALLLMVNADDNPLAFTPPTFEHLSPWQCLIHTQTEPLDAQISTTRYLLQDRSLMLFHADLIFKK

SEQ ID NO: 317 - protein, 739 aa, synthetic construct
MQKPTADDIKLNPDAKHNPSQNEKWALGAGEPFPLGASVEGDGVNFALFSANATAVELCLFDDNGEQEIARIALTEQTQQIWHVFVHGLKAGQLYGYRVYGSYEPLLGHRFNPNKLLLDPYARQLVGEYSDHESNYGYELNHKDEDLSFSKLDNAAHVPKCKVVDIRPLIDIAAKQSIQPVATRFQPNPLERSIIYEMHVKGFTAAHPEIEPNKRATFAGLATQPAIDYLAELGVTSVELLPVQAFFTEPFLLEKKLTNYWGYNSIGFFAPEPSYLSSDDIGEFRAMVDALHGAGIEVILDVVYNHSAEGSRLGPTFSFRGIDNLSYYRLHPNDKRFYINDTGCGNTLNINHPRMLQLVLDSLRYWVEIMGVDGFRFDLAACLGREAYGFDPGSGFFDALLQDPVLCRVKLIAEPWDIGPGGYQLGNFPVAFSEWNDRYRDTMRRFWRGDHSMLPEFARRFHGSGDFFEHGGRPPATSLNFLTSHDGFTLKDLVSYTQRHNLANGEDNRDGHQENFSHHYGIEGETEDSAILALRSRQQRNLLTTLFLSQGVPMLLSGDETGRTQQGNNNAYCQDNELNWFDWSAKGMDTELLAFTQQLIALRKRFPLLCAKRFIHEQLLAESLADTGARLDWFSRQGEQMTKSLWTESMCRSLSVVLSGDLEGQGKQQALLLMVNADDNPLAFTPPTFEHLSPWQCLIHTQTEPLDAQISTTRYLLQDRSLMLFHADLIFKKHHHHHH

SEQ ID NO: 318 - protein, 699 aa, synthetic construct
MKEISEIVQIVESGVGNAHPLGATLTHDGSNFAVYAPDAKAVVLCFFNCDTEEAISEHPLPEKTGDVWHGHFSGVSAGQYYGYRVERGEVGLHAVPTDKLLIDPYAKKISRAIKWDARQYKHDSQFMIPKCIVIDHNDYATNHARPPVIPKHKRVVYEAHVKGLTKLHPEVPKAHRGKFIGAAHPSVIKHIKALGVTTVQFMPLCSFMPEPFITDKGLTNYWGYNPVNFFAPEPRYGVSDALAELKSMIDAYHSAGLEVIVDVVFNHTAEAGGGGPILSYKGFCPNQAYLLEQTKNGELVYSNHSGCGNTVNTAQPFMMGLILDAMRHWVSVIGVDGFRFDLAVCLGREPQQYNKKSGLLRAISSDPVLKDKVLLAEPWDIGPNGYQVGNFPSPWLEVNDKYRDTVRAFWRGDGGVTADFATRLMGSRDIFHKGRRHISTSVNNVTYHDGFTLHDMVTYAERHNLDNLEENRDGHGHNLSANYGVEGETNDERILAMRERQKRNLFATLIFSQGTPHILGGDELSRTQNGNNNAYCQDNPISWMNWELNKRKQDFLSFCQYVVRLRQSSLLLSELKLHDDTFTLSRNVKEINWYKPDGSDKASEDWNAHHNKAFGVEIKGCVMSQREPDQKPEHWFLCVNASDSDVRFHLPTVLPKGGWTMHLDTRYSSLEEQPSICIQKVFLQASKSLTLFSFSQFSE

SEQ ID NO: 319 - protein, 705 aa, synthetic construct
MKEISEIVQIVESGVGNAHPLGATLTHDGSNFAVYAPDAKAVVLCFFNCDTEEAISEHPLPEKTGDVWHGHFSGVSAGQYYGYRVERGEVGLHAVPTDKLLIDPYAKKISRAIKWDARQYKHDSQFMIPKCIVIDHNDYATNHARPPVIPKHKRVVYEAHVKGLTKLHPEVPKAHRGKFIGAAHPSVIKHIKALGVTTVQFMPLCSFMPEPFITDKGLTNYWGYNPVNFFAPEPRYGVSDALAELKSMIDAYHSAGLEVIVDVVFNHTAEAGGGGPILSYKGFCPNQAYLLEQTKNGELVYSNHSGCGNTVNTAQPFMMGLILDAMRHWVSVIGVDGFRFDLAVCLGREPQQYNKKSGLLRAISSDPVLKDKVLLAEPWDIGPNGYQVGNFPSPWLEVNDKYRDTVRAFWRGDGGVTADFATRLMGSRDIFHKGRRHISTSVNNVTYHDGFTLHDMVTYAERHNLDNLEENRDGHGHNLSANYGVEGETNDERILAMRERQKRNLFATLIFSQGTPHILGGDELSRTQNGNNNAYCQDNPISWMNWELNKRKQDFLSFCQYVVRLRQSSLLLSELKLHDDTFTLSRNVKEINWYKPDGSDKASEDWNAHHNKAFGVEIKGCVMSQREPDQKPEHWFLCVNASDSDVRFHLPTVLPKGGWTMHLDTRYSSLEEQPSICIQKVFLQASKSLTLFSFSQFSEHHHHHH

SEQ ID NO: 320 - protein, 720 aa, synthetic construct
MPYKSLTLRPGSPFPLGATWDGKGTNFALYSENATGVELCLFDAEGHETRFPLTEQTAFVWHGYLPGIQPGQRYGYRVHGEYAPEKGLRFNPNVVLLDPYAKALDGTEQFDRGVFGYVAGGEDDSQMQEEEQRGAPLGLVVDPMFNWVGDQKPGIPFHQSVIYEAHVKGLTMTHPDVPEELRGTYAGVATPAILDYLRDLGITAIEFLPVHQHVDDPFLLDKGLTNYWGYSTLNFFAPDVRYSAEARKGNPSGAVPEFKNMVRALHDAGIEVILDVVYNHTAEGNHMGPTMSFKGIDNPTYYRLVADDQRFYFDYTGTGNSLNVRHPQTLQLIMDSLRYWVTEMHVDGFRFDLASTLARGLHEVDQLSGFFTIIHQDPIISQVKLIAEPWDVGEGGYQVGNFPVNWAEWNGIYRDDMRSFWKGEGGLASEIGYRITGSSDLYEFNGRKPYASINFVTAHDGFTLRDSVTYEQKHNEANGEGNNDGHNHNITWNCGVEGPTDDPEINRLRGQQMRNFLATLLLGQGTPMILGGDEFGRTQGGNNNAYCQDNDISWYDWEKVDEELLAFTRKLIALRKAHPSLHRRKFFAGRNIRGEDVRDIVWLRFDGAEMSDEDWNNPQTQSLGMFLAGDGLADVDAEGKPLTDDHLLLLLSSSYVDLPFKMPDLGGCGEWDLLLDTSDDGAEEKVAAGGETTLRGRSVKLYRCQVPEKELEKLAEPAEA

SEQ ID NO: 321 - protein, 726 aa, synthetic construct
MPYKSLTLRPGSPFPLGATWDGKGTNFALYSENATGVELCLFDAEGHETRFPLTEQTAFVWHGYLPGIQPGQRYGYRVHGEYAPEKGLRFNPNVVLLDPYAKALDGTEQFDRGVFGYVAGGEDDSQMQEEEQRGAPLGLVVDPMFNWVGDQKPGIPFHQSVIYEAHVKGLTMTHPDVPEELRGTYAGVATPAILDYLRDLGITAIEFLPVHQHVDDPFLLDKGLTNYWGYSTLNFFAPDVRYSAEARKGNPSGAVPEFKNMVRALHDAGIEVILDVVYNHTAEGNHMGPTMSFKGIDNPTYYRLVADDQRFYFDYTGTGNSLNVRHPQTLQLIMDSLRYWVTEMHVDGFRFDLASTLARGLHEVDQLSGFFTIIHQDPIISQVKLIAEPWDVGEGGYQVGNFPVNWAEWNGIYRDDMRSFWKGEGGLASEIGYRITGSSDLYEFNGRKPYASINFVTAHDGFTLRDSVTYEQKHNEANGEGNNDGHNHNITWNCGVEGPTDDPEINRLRGQQMRNFLATLLLGQGTPMILGGDEFGRTQGGNNNAYCQDNDISWYDWEKVDEELLAFTRKLIALRKAHPSLHRRKFFAGRNIRGEDVRDIVWLRFDGAEMSDEDWNNPQTQSLGMFLAGDGLADVDAEGKPLTDDHLLLLLSSSYVDLPFKMPDLGGCGEWDLLLDTSDDGAEEKVAAGGETTLRGRSVKLYRCQVPEKELEKLAEPAEAHHHHHH

SEQ ID NO: 322 - protein, 776 aa, synthetic construct
MKCPKILAALLGCAVLAGVPAMPAHAAINSMSLGASYDAQQANITFRVYSSQATRIVLYLYSAGYGVQESATYTLSPAGSGVWAVTVPVSSIKAAGITGAVYYGYRAWGPNWPYASNWGKGSQAGFVSDVDANGDRFNPNKLLLDPYAQEVSQDPLNPSNQNGNVFASGASYRTTDSGIYAPKGVVLVPSTQSTGTKPTRAQKDDVIYEVHVRGFTEQDTSIPAQYRGTYYGAGLKASYLASLGVTAVEFLPVQETQNDANDVVPNSDANQNYWGYMTENYFSPDRRYAYNKAAGGPTAEFQAMVQAFHNAGIKVYMDVVYNHTAEGGTWTSSDPTTATIYSWRGLDNATYYELTSGNQYFYDNTGIGANFNTYNTVAQNLIVDSLAYWANTMGVDGFRFDLASVLGNSCLNGAYTASAPNCPNGGYNFDAADSNVAINRILREFTVRPAAGGSGLDLFAEPWAIGGNSYQLGGFPQGWSEWNGLFRDSLRQAQNELGSMTIYVTQDANDFSGSSNLFQSSGRSPWNSINFIDVHDGMTLKDVYSCNGANNSQAWPYGPSDGGTSTNYSWDQGMSAGTGAAVDQRRAARTGMAFEMLSAGTPLMQGGDEYLRTLQCNNNAYNLDSSANWLTYSWTTDQSNFYTFAQRLIAFRKAHPALRPSSWYSGSQLTWYQPSGAVADSNYWNNTSNYAIAYAINGPSLGDSNSIYVAYNGWSSSVTFTLPAPPSGTQWYRVTDTCDWNDGASTFVAPGSETLIGGAGTTYGQCGQSLLLLISK

SEQ ID NO: 323 - protein, 756 aa, synthetic construct
AINSMSLGASYDAQQANITFRVYSSQATRIVLYLYSAGYGVQESATYTLSPAGSGVWAVTVPVSSIKAAGITGAVYYGYRAWGPNWPYASNWGKGSQAGFVSDVDANGDRFNPNKLLLDPYAQEVSQDPLNPSNQNGNVFASGASYRTTDSGIYAPKGVVLVPSTQSTGTKPTRAQKDDVIYEVHVRGFTEQDTSIPAQYRGTYYGAGLKASYLASLGVTAVEFLPVQETQNDANDVVPNSDANQNYWGYMTENYFSPDRRYAYNKAAGGPTAEFQAMVQAFHNAGIKVYMDVVYNHTAEGGTWTSSDPTTATIYSWRGLDNATYYELTSGNQYFYDNTGIGANFNTYNTVAQNLIVDSLAYWANTMGVDGFRFDLASVLGNSCLNGAYTASAPNCPNGGYNFDAADSNVAINRILREFTVRPAAGGSGLDLFAEPWAIGGNSYQLGGFPQGWSEWNGLFRDSLRQAQNELGSMTIYVTQDANDFSGSSNLFQSSGRSPWNSINFIDVHDGMTLKDVYSCNGANNSQAWPYGPSDGGTSTNYSWDQGMSAGTGAAVDQRRAARTGMAFEMLSAGTPLMQGGDEYLRTLQCNNNAYNLDSSANWLTYSWTTDQSNFYTFAQRLIAFRKAHPALRPSSWYSGSQLTWYQPSGAVADSNYWNNTSNYAIAYAINGPSLGDSNSIYVAYNGWSSSVTFTLPAPPSGTQWYRVTDTCDWNDGASTFVAPGSETLIGGAGTTYGQCGQSLLLLISKHHHHHH

SEQ ID NO: 324 - protein, 790 aa, synthetic construct
MIRPGSATPLGVWWDGEGVNVAVVAPGADAVDFCVFDGQNGSGGQNGSGATGRTDLGTDPVRGTDGGAETTSGGETRYRLPERDGGVWHGYIPGVGPGQAYGLRAHGPYAPGRGERYNPAKLLLDPYARRVTGSFVPHPAVHGYVAGDPYGQDPDDRDSAPYVPKGVVTGPVTRPPAGSVTGSAAGSVTGPDPAANRPRTAWADTILYELHVRGFTMLHPGVPERLRGTYAGLAHPAVVDHLLRIGVTAVELLPVHAHISETTLLENGRSNYWGYNTLAFFAPHPGYAATDDPVAEFRAMVAALHDAGIEVLLDVVYNHTAEGSERGPTLSLRGLDNIAYYRVEPTDPRRYRDVTGCGNTVDATSPHVVRLICDSLRYWVSEMGVDGFRFDLATALARSPDGFEPAAPLLTAIQADPLLSSVKLIAEPWDLGWGGYQVGAFPAPWAEWNGRFRDTLRDIFSDRTGSVADLGYRITGSSDIYEHSGRRPWASVNFVTAHDGFPLADLVSYNEKHNEANGEGNRDGESENRSSNHGAEGPTDDPKILTNRRRARRALISTLLLSAGVPMLLAGDELGRSQGGNNNAYCQDNAVSWLAWPNGTAGTPEAGGPGEVPAGEVPAADPAGPDPALVTLVGGLSRLRRAAPVLRRQRFFRGGAPTPQRLPDITWFRQDGAVMSAADWNAPQVAILVAHLAGEGIEWTDAAGTPITGESLLLVIHPEGEDRTVVLPGAPWATWYDLLLDTAADDLAGFPDTITEPRRTLAAGAQLDVAGRTVLILRALGGSDCSVAAE

SEQ ID NO: 325 - protein, 796 aa, synthetic construct
MIRPGSATPLGVWWDGEGVNVAVVAPGADAVDFCVFDGQNGSGGQNGSGATGRTDLGTDPVRGTDGGAETTSGGETRYRLPERDGGVWHGYIPGVGPGQAYGLRAHGPYAPGRGERYNPAKLLLDPYARRVTGSFVPHPAVHGYVAGDPYGQDPDDRDSAPYVPKGVVTGPVTRPPAGSVTGSAAGSVTGPDPAANRPRTAWADTILYELHVRGFTMLHPGVPERLRGTYAGLAHPAVVDHLLRIGVTAVELLPVHAHISETTLLENGRSNYWGYNTLAFFAPHPGYAATDDPVAEFRAMVAALHDAGIEVLLDVVYNHTAEGSERGPTLSLRGLDNIAYYRVEPTDPRRYRDVTGCGNTVDATSPHVVRLICDSLRYWVSEMGVDGFRFDLATALARSPDGFEPAAPLLTAIQADPLLSSVKLIAEPWDLGWGGYQVGAFPAPWAEWNGRFRDTLRDIFSDRTGSVADLGYRITGSSDIYEHSGRRPWASVNFVTAHDGFPLADLVSYNEKHNEANGEGNRDGESENRSSNHGAEGPTDDPKILTNRRRARRALISTLLLSAGVPMLLAGDELGRSQGGNNNAYCQDNAVSWLAWPNGTAGTPEAGGPGEVPAGEVPAADPAGPDPALVTLVGGLSRLRRAAPVLRRQRFFRGGAPTPQRLPDITWFRQDGAVMSAADWNAPQVAILVAHLAGEGIEWTDAAGTPITGESLLLVIHPEGEDRTVVLPGAPWATWYDLLLDTAADDLAGFPDTITEPRRTLAAGAQLDVAGRTVLILRALGGSDCSVAAEHHHHHH

SEQ ID NO: 326 - protein, 1576 aa, synthetic construct
MSVSRFSTPVEPEHPPFMVWNLDLNEDGTTPHDKSFIRIPSVLNQSCAVRFRILIGSTASIDAVLHTNYPLDGSDYVRTKFHSKRFNFNNSTELICEFRIQRPGPYQYYVTYKSVDDEESDYTDCISCDSDEVPTLERVYRVFKDRRTPTSYFLVDPQLTLGGQHLALDGIALQSVSPKWLGPMKGWSKHLANSSKMGYNMLHFIPMQQRGGSDSPYSLYNQLELSDDLFEGEKLSKEEKDQRLRTVCLEMHHKHHLLGITDMVWNHTAYNSEWLCDHPEAGFNLVNSPHLRSAYELDAKLCEFSKNLPEYGFDRMVSTVAQVDSLMEGVNTHVIQPLKLWEFYVVDVEGTVQAVSDAWDKAGVEMSADDIAKATELQGDERNEWLQTFVINNDPHTLSTRHGRTIDSERAVAVLRLLNGNDKKQGLEQLRRLLDLINLPYYREYDSDVARIRKNVGERVKYERLDRGSWKFGKPVDDDFKIVDPLFTTVTPRGGDSGAGKVNEDLYHLANNGWIWGGNPLDNFAGPQSKAYLQREVIVWGDCVKLNYGTQASDNPWLWEHMRQYTLSMAKAFHGFRIDNCHSTPIELAEYLLDQARKVKPDLYVIAELFTGSEATDQIFVQRLGINSLIREAMQAWDSHEMSRLAHRHGGRPVGSLAVDCLGEPGFFTDDEHGGTRVDGIVAPLSASLPHAIFFDCTHDNEVPAQKRTMQDALPNAAIVAMTACATGSNRGYDELYPELLNIVHESRQYAPLEDPLSVGLGEAKAKLNQLHAEMAQFQEVHVHHENEFVTVQRMHPVTRHGVLMVAHCAFRGASENSHFENPRLYGTAVHPEFAYRLRPGSGGSNGEGNSDDGLIHGLPSTLEKLDAPGVFEREDEHGMYTELRLPKDFGPGSVLVVRTQLVDFKPNLDWKIRTSADDAVARLDLAALNAALYRCDAEERDTIGEGVYDVPGLGPLPYCGLQGWHTHLRHIIPNNDLGHPLCGHLRQGTWALDYVVRRLHAYTEYHAPLAILAAWFEERWALVKRVPNFLMPRYFALTVHTAYCALIRRALALMPGDVISSSRFTRELALTSVQLVGHVNSAALRPVCQDKARTSMSAGLAHFSTHHMRCWGRDVFIALEGLLLVTGRHQEAREHILAFGSTLKHGLIPNLLDSGRYPRYNARDATWFWLQAVQTYCNHADNGLELLDEPVARRFPDGETFVEFDSDQAFSTTSTVAELIQEILQRHAQGIRFREWNAGPRLDEHMRDAGFDIEVSVDFASTGFVSGGNQWNCGTWMDKMGSSDKAGIRGVPGTPRDGADIEIVGLQKSALRWVSSLHEMERFPFEGVTTSSGKRVTYAEWDALVQRSFEKHFWVPLSAADDAEYHVDTALVNRRGIYRDTYGSTQAWADYQFRPNIAVAMVVAPELFDTGHALVCLHKMRAVLSGPLGMRTLDPSDMRYRPDYDNSNDSDDALVAHGINYHQGPEWLWPTGYYLAAQLEFMRHAVNQRSTGGKATHALRTIFHELHAHMVALKHHIANAPYAGLPELTNRDGSYCRDSCETQAWSSACLLMALQRMMDLEQKVHRLIELSDIVQ

SEQ ID NO: 327 - protein, 1582 aa, synthetic construct
MSVSRFSTPVEPEHPPFMVWNLDLNEDGTTPHDKSFIRIPSVLNQSCAVRFRILIGSTASIDAVLHTNYPLDGSDYVRTKFHSKRFNFNNSTELICEFRIQRPGPYQYYVTYKSVDDEESDYTDCISCDSDEVPTLERVYRVFKDRRTPTSYFLVDPQLTLGGQHLALDGIALQSVSPKWLGPMKGWSKHLANSSKMGYNMLHFIPMQQRGGSDSPYSLYNQLELSDDLFEGEKLSKEEKDQRLRTVCLEMHHKHHLLGITDMVWNHTAYNSEWLCDHPEAGFNLVNSPHLRSAYELDAKLCEFSKNLPEYGFDRMVSTVAQVDSLMEGVNTHVIQPLKLWEFYVVDVEGTVQAVSDAWDKAGVEMSADDIAKATELQGDERNEWLQTFVINNDPHTLSTRHGRTIDSERAVAVLRLLNGNDKKQGLEQLRRLLDLINLPYYREYDSDVARIRKNVGERVKYERLDRGSWKFGKPVDDDFKIVDPLFTTVTPRGGDSGAGKVNEDLYHLANNGWIWGGNPLDNFAGPQSKAYLQREVIVWGDCVKLNYGTQASDNPWLWEHMRQYTLSMAKAFHGFRIDNCHSTPIELAEYLLDQARKVKPDLYVIAELFTGSEATDQIFVQRLGINSLIREAMQAWDSHEMSRLAHRHGGRPVGSLAVDCLGEPGFFTDDEHGGTRVDGIVAPLSASLPHAIFFDCTHDNEVPAQKRTMQDALPNAAIVAMTACATGSNRGYDELYPELLNIVHESRQYAPLEDPLSVGLGEAKAKLNQLHAEMAQFQEVHVHHENEFVTVQRMHPVTRHGVLMVAHCAFRGASENSHFENPRLYGTAVHPEFAYRLRPGSGGSNGEGNSDDGLIHGLPSTLEKLDAPGVFEREDEHGMYTELRLPKDFGPGSVLVVRTQLVDFKPNLDWKIRTSADDAVARLDLAALNAALYRCDAEERDTIGEGVYDVPGLGPLPYCGLQGWHTHLRHIIPNNDLGHPLCGHLRQGTWALDYVVRRLHAYTEYHAPLAILAAWFEERWALVKRVPNFLMPRYFALTVHTAYCALIRRALALMPGDVISSSRFTRELALTSVQLVGHVNSAALRPVCQDKARTSMSAGLAHFSTHHMRCWGRDVFIALEGLLLVTGRHQEAREHILAFGSTLKHGLIPNLLDSGRYPRYNARDATWFWLQAVQTYCNHADNGLELLDEPVARRFPDGETFVEFDSDQAFSTTSTVAELIQEILQRHAQGIRFREWNAGPRLDEHMRDAGFDIEVSVDFASTGFVSGGNQWNCGTWMDKMGSSDKAGIRGVPGTPRDGADIEIVGLQKSALRWVSSLHEMERFPFEGVTTSSGKRVTYAEWDALVQRSFEKHFWVPLSAADDAEYHVDTALVNRRGIYRDTYGSTQAWADYQFRPNIAVAMVVAPELFDTGHALVCLHKMRAVLSGPLGMRTLDPSDMRYRPDYDNSNDSDDALVAHGINYHQGPEWLWPTGYYLAAQLEFMRHAVNQRSTGGKATHALRTIFHELHAHMVALKHHIANAPYAGLPELTNRDGSYCRDSCETQAWSSACLLMALQRMMDLEQKVHRLIELSDIVQHHHHHH

SEQ ID NO: 328 - protein, 2418 aa, synthetic construct
MSSPRQVYLLPLKDDGSPDVPGGYIYLPAPSDPSYLLRFVIEGTSSICREGQLWVNIPEDGHPFDRSAFRSFRLSPDFNKNIQIDVPITSPGSFAFYVTFSPLPEFSVSQTATPEPTRTPTYYIDVSPKLTLRGRDLPLNALSIYSVISKFLGKYPTDWDKHLNGISQRNYNMIHFTPLMKRGDSNSPYSIFDQLQFDDAVFPNGEADVANLVTKMEEKHGLLSLTDVVWNHTANNSKWLEEHPEAGYSVETAPWLESALDLDNALLEIGENLASLGLPTEFKSVDDLVAVMNAIREQVIHKLKLWEFYVVNVAADTQKVIQQWQTSKSIDITSDEWSQHKLKDFGNWTLEEQAIFVREKAIPTSKQLLGRYSRAVEPKFGAAILTVLLGPSDAASSDTTAAEKAISKLLDEVNLPFYKEYDADVAEIMNQLFNRIKYLRIDSHGPKLGPVTEQNPLIETYFTRLPLNDTTKKHDSKALALVNNGWIWNADALRDNAGPFSRSYLRREVIVWGDCVKLRYGSSPEDNPFLWEFMTKYTRLMAKYFSGFRIDNCHSTPLVVAEYLLDEARKVRPNLTVFAELFTGSEEADYIFVKRLGINALIREAMQAWSTGELSRLVHRHGGRPIGSFGVDLPSSGSSHAIASSGIDGGKEKVSHIRPNPVQALFMDCTHDNEMPAQKRTAIDTLPNAALVAMCASAIGSVIGYDEIYPKLVDLVHETRLYSSRFSESSKVDLDSLEGGIGGVKKLLNELHTIMGSEGYDETHIHHDGEYITVHRVHPKTRKGVFLIAHTAFPGQDSKAILAPTRLVGTRAKHIGTWRLEVDSSESTRKEVSEDKSYLRGLPSQVHAIEGTKAEQDGNDTVISVLESLVAGSISLYETSIPSAEHASGLDVHITEGVDEAFSNLGLVDLNFVLYRCEAEERDSSNGQDGVYSIPNHGSLVYAGLQGWWSVLENVIKYNELGHPLCDHLRNGQWALDFIVGRLEKVAKTEGYSALHKPAAWLREKFDAVRSLPSFLLPRYFAIIVQTAYNAAWKRGIQLLGGNIQKGQEFIHQLSMVSVQMTGFVNSASLWPTKRVPSLAAGLPHFAVDWARCWGRDVFISLRGLLLCTSRFDDAREHIFAFASVLKHGMIPNLLSSGRLPRYNSRDSVWFFLQSIQDYTLMAPDGIKLLDEKVPRRFLPYDDTWFPFDDPRAYSKHSTISEVIQEVFQRHARGLSFREYNAGPDLDVQMKPEGFEINVQVDWDTGLIFGGSQFNCGTWQDKMGESEKAKNKGVPGTPRDGAAIEITGLVYSALNWVAKLHEQGLYQHSGVDLGEGKSVTFKSWAAKIKENFERCYYIPSNPEEDASYVVDRNVVNRRGIYKDLYKSGKPYEDYQLRSNFPIAMTVAPDLFTPSKALSALALADEVLVGPVGMATLDPSDLNYRPNYNNSEDSEDFATSKGRNYHQGPEWVWQRGFFLRAFLHFDLARRATREERTEAYQQVTRRLEGCKKALRESPWRGLTELSNKDGAYCADSSPTQAWSAGCLLDLYYDASQYTQDDSAVQNEVDMRPTVLKSSACPYALIIQDIRQTHPSAEHNLTRVMSLSQSAAAAMLTAVDAPIEVHEPSWGDQTNPRDFLTQFVISMTIGLGAFLAFCVLRPKWTELYAARRRQRCAASHLPELPDSFFGWIPVLYRITEEQVLESAGLDAFVFLTFLKFAIRFLSGVFFFALVIILPMHYRNTGKSGVPGWDDDDEPVDVFDGDKDKKKIISDPNYLWMYVVFTYIFTGLAVYMLVQDTNKVIRIRQQYLGSQTSTTDRTIRLSGIPHDMGSEEKIREFIEGLQIGKVQSVTLCKDWRELDRLIDERLKILRNLEWAWTKHLGYKRVKANANRLPLLQQHARGSSIVSEEDSERIQLISDSTPAHVLDYAHKRPTTRIWYGPFKLRYRNVDAIDYYEEKLRRIDEQIQLARQKEYPPTEIAFVTMESIAASQMVVQAILDPQPMQLLARLAPAPADVVWKNTYIPRSRRMMQSWSITVVIGFLTVFWSVLLIPVAYLLEYETLHKVFPQLADALSRNPLAKSLVQTGLPTLVLSLLTIAVPYLYSWLSHQQGMMSRGDIELSVISKTFFFSFFNLFLVFTVFGTATTFFGFWENLRDAFKDATTIAFALAKTLENFAPFYINFLCLQGIGLLPFRLLEFGSVAMYPINFLTAKTPRDYAELNTPPVFSYGYSIPQTILSLIICVVYSVFPSSWLICLFGLIYFTIGKFIYKYQLLYAMDHQQHSTGRAWPMICSRVLMGLIVFQLAMIGVLALRRAITRSLLIVPLLMATVWFSYFFSRNYEPLMKFIALKSIDRERPGGGDISPSPSSTFSPPSGLERDAFPIRIGGQELGLRLKKYVNPSLILPLHDAWLPGRSTAPGYQHDSGVEETPNVAAEESV

SEQ ID NO: 329 - protein, 2424 aa, synthetic construct
MSSPRQVYLLPLKDDGSPDVPGGYIYLPAPSDPSYLLRFVIEGTSSICREGQLWVNIPEDGHPFDRSAFRSFRLSPDFNKNIQIDVPITSPGSFAFYVTFSPLPEFSVSQTATPEPTRTPTYYIDVSPKLTLRGRDLPLNALSIYSVISKFLGKYPTDWDKHLNGISQRNYNMIHFTPLMKRGDSNSPYSIFDQLQFDDAVFPNGEADVANLVTKMEEKHGLLSLTDVVWNHTANNSKWLEEHPEAGYSVETAPWLESALDLDNALLEIGENLASLGLPTEFKSVDDLVAVMNAIREQVIHKLKLWEFYVVNVAADTQKVIQQWQTSKSIDITSDEWSQHKLKDFGNWTLEEQAIFVREKAIPTSKQLLGRYSRAVEPKFGAAILTVLLGPSDAASSDTTAAEKAISKLLDEVNLPFYKEYDADVAEIMNQLFNRIKYLRIDSHGPKLGPVTEQNPLIETYFTRLPLNDTTKKHDSKALALVNNGWIWNADALRDNAGPFSRSYLRREVIVWGDCVKLRYGSSPEDNPFLWEFMTKYTRLMAKYFSGFRIDNCHSTPLVVAEYLLDEARKVRPNLTVFAELFTGSEEADYIFVKRLGINALIREAMQAWSTGELSRLVHRHGGRPIGSFGVDLPSSGSSHAIASSGIDGGKEKVSHIRPNPVQALFMDCTHDNEMPAQKRTAIDTLPNAALVAMCASAIGSVIGYDEIYPKLVDLVHETRLYSSRFSESSKVDLDSLEGGIGGVKKLLNELHTIMGSEGYDETHIHHDGEYITVHRVHPKTRKGVFLIAHTAFPGQDSKAILAPTRLVGTRAKHIGTWRLEVDSSESTRKEVSEDKSYLRGLPSQVHAIEGTKAEQDGNDTVISVLESLVAGSISLYETSIPSAEHASGLDVHITEGVDEAFSNLGLVDLNFVLYRCEAEERDSSNGQDGVYSIPNHGSLVYAGLQGWWSVLENVIKYNELGHPLCDHLRNGQWALDFIVGRLEKVAKTEGYSALHKPAAWLREKFDAVRSLPSFLLPRYFAIIVQTAYNAAWKRGIQLLGGNIQKGQEFIHQLSMVSVQMTGFVNSASLWPTKRVPSLAAGLPHFAVDWARCWGRDVFISLRGLLLCTSRFDDAREHIFAFASVLKHGMIPNLLSSGRLPRYNSRDSVWFFLQSIQDYTLMAPDGIKLLDEKVPRRFLPYDDTWFPFDDPRAYSKHSTISEVIQEVFQRHARGLSFREYNAGPDLDVQMKPEGFEINVQVDWDTGLIFGGSQFNCGTWQDKMGESEKAKNKGVPGTPRDGAAIEITGLVYSALNWVAKLHEQGLYQHSGVDLGEGKSVTFKSWAAKIKENFERCYYIPSNPEEDASYVVDRNVVNRRGIYKDLYKSGKPYEDYQLRSNFPIAMTVAPDLFTPSKALSALALADEVLVGPVGMATLDPSDLNYRPNYNNSEDSEDFATSKGRNYHQGPEWVWQRGFFLRAFLHFDLARRATREERTEAYQQVTRRLEGCKKALRESPWRGLTELSNKDGAYCADSSPTQAWSAGCLLDLYYDASQYTQDDSAVQNEVDMRPTVLKSSACPYALIIQDIRQTHPSAEHNLTRVMSLSQSAAAAMLTAVDAPIEVHEPSWGDQTNPRDFLTQFVISMTIGLGAFLAFCVLRPKWTELYAARRRQRCAASHLPELPDSFFGWIPVLYRITEEQVLESAGLDAFVFLTFLKFAIRFLSGVFFFALVIILPMHYRNTGKSGVPGWDDDDEPVDVFDGDKDKKKIISDPNYLWMYVVFTYIFTGLAVYMLVQDTNKVIRIRQQYLGSQTSTTDRTIRLSGIPHDMGSEEKIREFIEGLQIGKVQSVTLCKDWRELDRLIDERLKILRNLEWAWTKHLGYKRVKANANRLPLLQQHARGSSIVSEEDSERIQLISDSTPAHVLDYAHKRPTTRIWYGPFKLRYRNVDAIDYYEEKLRRIDEQIQLARQKEYPPTEIAFVTMESIAASQMVVQAILDPQPMQLLARLAPAPADVVWKNTYIPRSRRMMQSWSITVVIGFLTVFWSVLLIPVAYLLEYETLHKVFPQLADALSRNPLAKSLVQTGLPTLVLSLLTIAVPYLYSWLSHQQGMMSRGDIELSVISKTFFFSFFNLFLVFTVFGTATTFFGFWENLRDAFKDATTIAFALAKTLENFAPFYINFLCLQGIGLLPFRLLEFGSVAMYPINFLTAKTPRDYAELNTPPVFSYGYSIPQTILSLIICVVYSVFPSSWLICLFGLIYFTIGKFIYKYQLLYAMDHQQHSTGRAWPMICSRVLMGLIVFQLAMIGVLALRRAITRSLLIVPLLMATVWFSYFFSRNYEPLMKFIALKSIDRERPGGGDISPSPSSTFSPPSGLERDAFPIRIGGQELGLRLKKYVNPSLILPLHDAWLPGRSTAPGYQHDSGVEETPNVAAEESVHHHHHH

SEQ ID NO: 330 - protein, 2043 aa, synthetic construct
MRSVLLRLDDEGSPIKNDAVHNGVFIFPSYAPPEGWTRGAPLFELVVQLNAAAPVSKKGILCTNVPKDGKTAFQRSNFQRYPLSTSFTKDTTIRVPIYSPGPYNLYIEYENEEQEKVKTPDHYFVVPPNLMVNDKYVPFNAINIQTVVSKWAGPLDNWDKLFRYINEKGYDMIHFTPLQERGSSNSPYSIRDQLRFDPNLFKDTESAISFIHRTTEKHNLLSLTDVVWNHTSDDSPWLRDHPDAGYNAETAPHLTSAIELDKALLEYSDKLADLGLPTELKSESDLKKLVDGIQDHVLGPLELWQYYVFDKKSSLEDLRKAHEIGGVSPSTIPDDTDVSDVKQLASFILKRASIHEKPILGDRFSNKLDPNKFLSYLLTLAGDDFNEIEKKASQIIDEINVSLYATFDDDIRSIKAQIADRARYLRLADNGPKLGPITKENRFTESYFTRFEGKDGKEYALANNGWIWGGNPLVDFASSESRAYIRREVIVWSDCVKLRYGKGPEDSPHVWKRMIEYTKNSAKAFSGFRLDNCHSTPLHVGEALLDAARSVNPDLYVVAELFTGSEEMDKIFVERLGINSLIREAMQAWSVEELSRLVHKHGGRPIGSLTWMPLDDFSFPCNKEPTAGKYSEGHSELEIPHVLAKQAPHALFMDCTHDNQTPAQVRTVGDTLSTAALVAFCSSAIGTTFGFDETYPELLDIVNETRTYDYDFKTGIAEARRKMNDVRDSLAAESKDAASDQEMYIHHEGQYITIQRYNTVTGEGWFLIARTKFTDSPGNQILSPVILKGTNVKYEFGYTLKKVGEYSKDDKYLTSIPTKLEEVSLPPVEHNGDEAIIRVDNNFVPGSIAVFSTKIANVDASLDSYVKKGAVEASLGLDLYDLNAILYRCEPEERDASNGMEGVYNIPNYGPLVYAGLQGWVSVLKQAVWRNDLAHPLCDHLREGFWAADYIVNRLEKYSKNSKNLHKFHAWLKSRVDAIRDVPYFLRPHYFALVVGVAYEAARFRVLRQLDDHIKTATNFVQSLALTSVQMCGYMNNTSLLPDKNIPCLAAGLPHFSNDYMRCWGRDVFISYRGLLIVPGRHEDAKQHILGFAKTLKHGLIPNLLDAGRNPRYNARDAAWFFARAVEEYVTHVPHGEQILDETVSRRFPLDDTYVSWDDKRAFSYETSIRDILYEILSRHAKGIKYREANAGPNLDSQMKDEGFNVEVYVDWNTGLVHGGSQWNCGTWMDKMGESEKAGNKGVPGTPRDGAAVELQGLLKSTLRFVNGLHKKGLFEHTEVEKPDGSKISLIEWESLIQENFEKCFFVPEDPAEDDHYEINPDVVNRRGIYKDLFKSGKEYEDYQLRANFPIAMCVAPELFTPKNALSAINKADEIIRGPVGMRTLDPSDWNYRPNYNNSEDSEDFATSKGRNYHQGPEWVWLFGYFIRAFLYFNHFADNGGEGSPTKELLTEVNKRISGHKKWIRESPWAGITELTNQNGALCHDSSPTQAWTSSLMLRNLSKSAARMAKKPAPLFGRSLSTLTLNTTTDSYPTNHFEKGTEPYLTPSFIDNQLVKSESQTWFDIHDPATNEVISKVPQSTPQELDDAIASAEKAFHEWKNYSIIKRQGIAFKFVELLKKNHDRLAGMIVLEQGKTFPDAKGDVLRGLQVAEAACNAPNDLMASSLEVATDMETKMVREPLGVIGSICPFNFPAMVPLWSLPLVLVTGNTAVIKPSERVPGAAMIIAQLAAEAGVPPGVLNIVHGKHDTVNKIIEDPRVKAINFVGGDKAGKYIYEKGTSLGKRVQANLGAKNHLIVLPDADKNHFINAVNGAAFGAAGQRCMAISVLVTVGEKTKQWVEEVASDAAKLVTGSGFDPKSDLGPLVNPESLTRAKEIINDSEKQGAKVILDGRNFKPSDPKFAKGNFLGPTILTNVKPGMRAYDEEIFAPVLSVVNVDTFDEAIALVNSNRYGNGVSLFTSAGAAAQKFTKEIACGQVGVNVPIPVPLPMFSFTGNKGSFLGDLNFYGKAGVTFLTQPKTITTLWRTSTVDQVLAPSTAMPTQH

SEQ ID NO: 331 - protein, 2049 aa, synthetic construct
MRSVLLRLDDEGSPIKNDAVHNGVFIFPSYAPPEGWTRGAPLFELVVQLNAAAPVSKKGILCTNVPKDGKTAFQRSNFQRYPLSTSFTKDTTIRVPIYSPGPYNLYIEYENEEQEKVKTPDHYFVVPPNLMVNDKYVPFNAINIQTVVSKWAGPLDNWDKLFRYINEKGYDMIHFTPLQERGSSNSPYSIRDQLRFDPNLFKDTESAISFIHRTTEKHNLLSLTDVVWNHTSDDSPWLRDHPDAGYNAETAPHLTSAIELDKALLEYSDKLADLGLPTELKSESDLKKLVDGIQDHVLGPLELWQYYVFDKKSSLEDLRKAHEIGGVSPSTIPDDTDVSDVKQLASFILKRASIHEKPILGDRFSNKLDPNKFLSYLLTLAGDDFNEIEKKASQIIDEINVSLYATFDDDIRSIKAQIADRARYLRLADNGPKLGPITKENRFTESYFTRFEGKDGKEYALANNGWIWGGNPLVDFASSESRAYIRREVIVWSDCVKLRYGKGPEDSPHVWKRMIEYTKNSAKAFSGFRLDNCHSTPLHVGEALLDAARSVNPDLYVVAELFTGSEEMDKIFVERLGINSLIREAMQAWSVEELSRLVHKHGGRPIGSLTWMPLDDFSFPCNKEPTAGKYSEGHSELEIPHVLAKQAPHALFMDCTHDNQTPAQVRTVGDTLSTAALVAFCSSAIGTTFGFDETYPELLDIVNETRTYDYDFKTGIAEARRKMNDVRDSLAAESKDAASDQEMYIHHEGQYITIQRYNTVTGEGWFLIARTKFTDSPGNQILSPVILKGTNVKYEFGYTLKKVGEYSKDDKYLTSIPTKLEEVSLPPVEHNGDEAIIRVDNNFVPGSIAVFSTKIANVDASLDSYVKKGAVEASLGLDLYDLNAILYRCEPEERDASNGMEGVYNIPNYGPLVYAGLQGWVSVLKQAVWRNDLAHPLCDHLREGFWAADYIVNRLEKYSKNSKNLHKFHAWLKSRVDAIRDVPYFLRPHYFALVVGVAYEAARFRVLRQLDDHIKTATNFVQSLALTSVQMCGYMNNTSLLPDKNIPCLAAGLPHFSNDYMRCWGRDVFISYRGLLIVPGRHEDAKQHILGFAKTLKHGLIPNLLDAGRNPRYNARDAAWFFARAVEEYVTHVPHGEQILDETVSRRFPLDDTYVSWDDKRAFSYETSIRDILYEILSRHAKGIKYREANAGPNLDSQMKDEGFNVEVYVDWNTGLVHGGSQWNCGTWMDKMGESEKAGNKGVPGTPRDGAAVELQGLLKSTLRFVNGLHKKGLFEHTEVEKPDGSKISLIEWESLIQENFEKCFFVPEDPAEDDHYEINPDVVNRRGIYKDLFKSGKEYEDYQLRANFPIAMCVAPELFTPKNALSAINKADEIIRGPVGMRTLDPSDWNYRPNYNNSEDSEDFATSKGRNYHQGPEWVWLFGYFIRAFLYFNHFADNGGEGSPTKELLTEVNKRISGHKKWIRESPWAGITELTNQNGALCHDSSPTQAWTSSLMLRNLSKSAARMAKKPAPLFGRSLSTLTLNTTTDSYPTNHFEKGTEPYLTPSFIDNQLVKSESQTWFDIHDPATNEVISKVPQSTPQELDDAIASAEKAFHEWKNYSIIKRQGIAFKFVELLKKNHDRLAGMIVLEQGKTFPDAKGDVLRGLQVAEAACNAPNDLMASSLEVATDMETKMVREPLGVIGSICPFNFPAMVPLWSLPLVLVTGNTAVIKPSERVPGAAMIIAQLAAEAGVPPGVLNIVHGKHDTVNKIIEDPRVKAINFVGGDKAGKYIYEKGTSLGKRVQANLGAKNHLIVLPDADKNHFINAVNGAAFGAAGQRCMAISVLVTVGEKTKQWVEEVASDAAKLVTGSGFDPKSDLGPLVNPESLTRAKEIINDSEKQGAKVILDGRNFKPSDPKFAKGNFLGPTILTNVKPGMRAYDEEIFAPVLSVVNVDTFDEAIALVNSNRYGNGVSLFTSAGAAAQKFTKEIACGQVGVNVPIPVPLPMFSFTGNKGSFLGDLNFYGKAGVTFLTQPKTITTLWRTSTVDQVLAPSTAMPTQHHHHHHH

SEQ ID NO: 332 - protein, 1829 aa, synthetic construct
MWSGFSRHFLYGYKAKIGYTGVQTVMLTISTFTSLSSIFLYYHIYGNPIKAMTPEEHGLHPPKYPWPHKGFLSSYDHKSLRRGYQVYKEVCSACHSLNLVAWRNLVGVTHTLDEAKAMAEEYEYEDGPDESVLRLIIGKLFDYMPKPYPNEEAARAANAGAFPPDLSLIIKARHGGCDYVFSLLTGYVDPPAGVVLADGMNYNPYFPNGQIAMARLLYDGLIEYEDGTPATTSQMAKDVVSFLNWAAEPEHDDRKRMGFQTLIILSTLFALNLWVKRFKWAPIKTSLFCMLYEFTKYLPFYRRLSSSEFKSVMAEIQIYSLKLSGTGSPLISGKYISLPPPFVPYYLRFEIDEITFIYYKSVLKHNFPPKGSKFNRDKWYSIPLDIDFSSIKNIDILVHTAGLFSYKIEYFQLPLWGLEEFDHSVPRKSTDEFYFTVLPALKINGSYISLNSLVIQSVVSKWMGPISEWDEIMHYIGKKKCYNMVHFTPLQSLGESNSPYSIYDQLSFSDDLFDVKLESKQKNNIVDKYLRKMEIEWGLLSITDVVWNHTARDSKWLQDHPEVGYNLHNSPHLIAAYELDASLLEFSANLGELGYPTNPKNIDDLLKIIDGIKNTVLEDLRLWEFYVVNVKESVKYTIDAYKKKKIVELDDKFDININWSLKEKVNAFIGVSAITNYNVLGERFAKRLVPEIGAAILYKLFGIDIEEQVLDDELTKILNEFNLEYYIEYDIDKKIIIDQIYNRSKYLRLDDNGPKKGEISRKNPLVETYFSLIERNKNTLLYSDRSLCLANNGWVWNSSLNDFAGQESKAYLLRQVIVWGDCVKLRYGKDPSDNPYLWDHMTKYTQMLARLFNGFRIDNCHSTPLHVGIYLLDKAREIRNDLYIVAELFTGNEEMDLVFMQKLGISSLIREAMQAWCPGELSRFIYKYGGKPIGSLDSISSKYHYLTSIKNCKFSNVVSVQNSQIHTLFMDCTHDNQMPAQKRTAYDTLPNAALVSMCNCSIGSVMGYDEIVPHYINIVMESRKYYYSLENVIGIESVKSVLNKIHVEMGKDNFEEIYVHHENSYITVHTVNPKNYHGYFLLAHTAFLMHENNKIRPIVLRGTKVDVVFSLSLEIESFEDSKDPDFIKGLSSTIIELDSPIITTGEDSEGFFTCIEVPKKFPPGSIFLLKTWIENVEDLDNFICTDLDNIMENFDLVDLNLIMYRCDQEERNTINDGCYNIPGFGDLVYCGFEGWMPVLKEIIRNNILDHPLCQHLRQGQWALDYVVQRLYKMENILSRLRQLADWLQMRFSRIKKIPNFLLPRYFITTLYLTSMAARNRAIGLMSPHIQNGHTFIKSLSLCSVQMHGIVKSASLDPFINKPCLAAGLPHFSYSWKRCWGRDVFISLRGLFLVTGRFDDAKRHILAFASVLKHGMVPNLLDSLKRPRYNSRDSVWFFLQAIQEYTKLVPDGIQILYERVKRRFPLDDSFVELDDYRAYSHESTILEVIHEIMQRHALGLHFREANAGLGLDTEMTDKGFDIDIYVDWKTGLIFGGSKYNCGTWMDKMGESLKAGNKGVPGTPRDGAAIEISGMLKSTLRWINELRRKNIYKWHCVNIKENGEFKDVTFEEWEKKIQNSFEFCYYIPFDPQEDSKYDVNISMINRRGIYKDVYRSNKEYEDYQLRPNFAIAMVVAPELFTFGYAVRAIEIADKVIRGPMGMLTLDPSDKDYRPYYINSYDSDDFATSKGRNYHQGPEWLWCTGYFLRAFLYFSRFYYNFTKNKDIVCYHVHNRLVTLMKEIEENPWAGLVELTNKNGEFCNDSNNTQSWSASTILDLYDDILKLRTLDKS

SEQ ID NO: 333 - protein, 1835 aa, synthetic construct
MWSGFSRHFLYGYKAKIGYTGVQTVMLTISTFTSLSSIFLYYHIYGNPIKAMTPEEHGLHPPKYPWPHKGFLSSYDHKSLRRGYQVYKEVCSACHSLNLVAWRNLVGVTHTLDEAKAMAEEYEYEDGPDESVLRLIIGKLFDYMPKPYPNEEAARAANAGAFPPDLSLIIKARHGGCDYVFSLLTGYVDPPAGVVLADGMNYNPYFPNGQIAMARLLYDGLIEYEDGTPATTSQMAKDVVSFLNWAAEPEHDDRKRMGFQTLIILSTLFALNLWVKRFKWAPIKTSLFCMLYEFTKYLPFYRRLSSSEFKSVMAEIQIYSLKLSGTGSPLISGKYISLPPPFVPYYLRFEIDEITFIYYKSVLKHNFPPKGSKFNRDKWYSIPLDIDFSSIKNIDILVHTAGLFSYKIEYFQLPLWGLEEFDHSVPRKSTDEFYFTVLPALKINGSYISLNSLVIQSVVSKWMGPISEWDEIMHYIGKKKCYNMVHFTPLQSLGESNSPYSIYDQLSFSDDLFDVKLESKQKNNIVDKYLRKMEIEWGLLSITDVVWNHTARDSKWLQDHPEVGYNLHNSPHLIAAYELDASLLEFSANLGELGYPTNPKNIDDLLKIIDGIKNTVLEDLRLWEFYVVNVKESVKYTIDAYKKKKIVELDDKFDININWSLKEKVNAFIGVSAITNYNVLGERFAKRLVPEIGAAILYKLFGIDIEEQVLDDELTKILNEFNLEYYIEYDIDKKIIIDQIYNRSKYLRLDDNGPKKGEISRKNPLVETYFSLIERNKNTLLYSDRSLCLANNGWVWNSSLNDFAGQESKAYLLRQVIVWGDCVKLRYGKDPSDNPYLWDHMTKYTQMLARLFNGFRIDNCHSTPLHVGIYLLDKAREIRNDLYIVAELFTGNEEMDLVFMQKLGISSLIREAMQAWCPGELSRFIYKYGGKPIGSLDSISSKYHYLTSIKNCKFSNVVSVQNSQIHTLFMDCTHDNQMPAQKRTAYDTLPNAALVSMCNCSIGSVMGYDEIVPHYINIVMESRKYYYSLENVIGIESVKSVLNKIHVEMGKDNFEEIYVHHENSYITVHTVNPKNYHGYFLLAHTAFLMHENNKIRPIVLRGTKVDVVFSLSLEIESFEDSKDPDFIKGLSSTIIELDSPIITTGEDSEGFFTCIEVPKKFPPGSIFLLKTWIENVEDLDNFICTDLDNIMENFDLVDLNLIMYRCDQEERNTINDGCYNIPGFGDLVYCGFEGWMPVLKEIIRNNILDHPLCQHLRQGQWALDYVVQRLYKMENILSRLRQLADWLQMRFSRIKKIPNFLLPRYFITTLYLTSMAARNRAIGLMSPHIQNGHTFIKSLSLCSVQMHGIVKSASLDPFINKPCLAAGLPHFSYSWKRCWGRDVFISLRGLFLVTGRFDDAKRHILAFASVLKHGMVPNLLDSLKRPRYNSRDSVWFFLQAIQEYTKLVPDGIQILYERVKRRFPLDDSFVELDDYRAYSHESTILEVIHEIMQRHALGLHFREANAGLGLDTEMTDKGFDIDIYVDWKTGLIFGGSKYNCGTWMDKMGESLKAGNKGVPGTPRDGAAIEISGMLKSTLRWINELRRKNIYKWHCVNIKENGEFKDVTFEEWEKKIQNSFEFCYYIPFDPQEDSKYDVNISMINRRGIYKDVYRSNKEYEDYQLRPNFAIAMVVAPELFTFGYAVRAIEIADKVIRGPMGMLTLDPSDKDYRPYYINSYDSDDFATSKGRNYHQGPEWLWCTGYFLRAFLYFSRFYYNFTKNKDIVCYHVHNRLVTLMKEIEENPWAGLVELTNKNGEFCNDSNNTQSWSASTILDLYDDILKLRTLDKSHHHHHH

SEQ ID NO: 334 - protein, 1563 aa, synthetic construct
MKGRSQLENTLLLRLDSDGVPIANNTLYGSGVLTLPSLPYKLEYINDDNPIVTLKIMIAAGSTISRDGLIWTNVPESSNVEFNRDHFRKIIIKSSIHSDCYIDLKLFRPGSFCFYVSYRNKKNLLKTTKKHYFILGPSLFINDTYLPLNSINCQTIISKWLPKSTSPEDNWQKIFKLIANKKYNMIHFTPLQQRGESNSPYSIYDQLMFDPDFFKENQKSVKLLIDDLEKTHNMLSLTDIVFNHTANNSFWLRKHPESGYNQFTAPHLNAAIELDKALLDFSSRLKSLNYPVDLKNDNDLLEILDGIKVHVLGQLKLWEYYVINIKETVNEVEAKFDEISITNGDEEDESLNDEILNNLENLAAYVRENYNVGNFDNLAERYSHKLNIEKLIILLKKSKKFEKFDEETNTFIINLLDEINLPLYRIYDDDVNEILEQLFNRIKYLRISDDGPKLGVITNKSPLTEPYFTRFVGENDGIEYALANNGWIWNGNPLIDFASKNSKAYLRREVIVWGDCVKLRYGKEPADSPYLWKRMSDYITMNAKIFHGFRIDNCHSTPLHVGEYFLDLARSHNPNLYVVAELFSGSETTDCLFVERLGISSLIREAMQASSEDELSNLIHRYGGRPIGSYKFVPMDTFAFSPDLELDEDYCTYSIKDKSIHCVSEIMIPRVLTCQPPHALFMDCTHDNKMPYEKRTIEDTLPNAALVAFCSSAIGSVYGYDEIYPELLDLVNEKRKYDLSGSINKNLNDEVGICKIKRKLNAIRKEIAISSIDIEDSEMHVHHEGQYITFHRTNSKTGKGYYLIVRTKFSDFNEDQMFPSVTLRATKCKFNCAYSLEKVDTSFSPDNDLLTGVKSKIVELKDFTIHVDADSNSVIELPENFPPGSIAIFETGYVGIDDTLDHLIRSGALKATSNLNFESLNAVLFRSESEELDVSGNTFGAYNIPNHGKLVYCGLQGWVSVLRDIMINNDLAHPLSVNLREGHWALDYILERLNLYNDKPGIRDVQAWLAERFDKIKQLPNFLVPSFFALTIGILYGCCRFKAMLLFPKYIGNANVFVQSLALTSIQMVSEVKSTSIFPNINVPCMCAGLPHFSTNYMRCWGRDVFISFRGLLLVTGRYETAKQHILGFAKTTKHGLIPNLLDAGRNPRYNARDAAWFFLQAIQDYITIVPEGHSILSEKVSRRFPLNDKYITYEDPEAFSYSSSIEEIIYEILSRHAKGIKFREANAGPNLDRVMKDEGFNIEIAVDWSTGLIHGGNQFNCGTWMDKNGESEKAKSKGVPGTPRDGAAVEINGLLKSTLRFVLKLISEGKFKKTDVTRQDGSKISFADWNDLLQENFEKNYYIPLDPKDDNEYNTDSNIVNRRGIYKDLCGSGKPFEDYQLRPNQFIAMAVAPELFSPEHAMSAIKVADENIRGKLGMRTLDPSDYNYRPYYNNSEDSDDFATSKGRNYHQGPEWVWCYGYFMRSFHTFNFLANPLSRNETENRPSSYLMQQIYLRLDDHRKAIIESPWAGLPELTNKDGEFCQDSSPTQAWSSGCILDVLYDVWVAFDSKWYAKQIKQNNP

SEQ ID NO: 335 - protein, 1569 aa, synthetic construct
MKGRSQLENTLLLRLDSDGVPIANNTLYGSGVLTLPSLPYKLEYINDDNPIVTLKIMIAAGSTISRDGLIWTNVPESSNVEFNRDHFRKIIIKSSIHSDCYIDLKLFRPGSFCFYVSYRNKKNLLKTTKKHYFILGPSLFINDTYLPLNSINCQTIISKWLPKSTSPEDNWQKIFKLIANKKYNMIHFTPLQQRGESNSPYSIYDQLMFDPDFFKENQKSVKLLIDDLEKTHNMLSLTDIVFNHTANNSFWLRKHPESGYNQFTAPHLNAAIELDKALLDFSSRLKSLNYPVDLKNDNDLLEILDGIKVHVLGQLKLWEYYVINIKETVNEVEAKFDEISITNGDEEDESLNDEILNNLENLAAYVRENYNVGNFDNLAERYSHKLNIEKLIILLKKSKKFEKFDEETNTFIINLLDEINLPLYRIYDDDVNEILEQLFNRIKYLRISDDGPKLGVITNKSPLTEPYFTRFVGENDGIEYALANNGWIWNGNPLIDFASKNSKAYLRREVIVWGDCVKLRYGKEPADSPYLWKRMSDYITMNAKIFHGFRIDNCHSTPLHVGEYFLDLARSHNPNLYVVAELFSGSETTDCLFVERLGISSLIREAMQASSEDELSNLIHRYGGRPIGSYKFVPMDTFAFSPDLELDEDYCTYSIKDKSIHCVSEIMIPRVLTCQPPHALFMDCTHDNKMPYEKRTIEDTLPNAALVAFCSSAIGSVYGYDEIYPELLDLVNEKRKYDLSGSINKNLNDEVGICKIKRKLNAIRKEIAISSIDIEDSEMHVHHEGQYITFHRTNSKTGKGYYLIVRTKFSDFNEDQMFPSVTLRATKCKFNCAYSLEKVDTSFSPDNDLLTGVKSKIVELKDFTIHVDADSNSVIELPENFPPGSIAIFETGYVGIDDTLDHLIRSGALKATSNLNFESLNAVLFRSESEELDVSGNTFGAYNIPNHGKLVYCGLQGWVSVLRDIMINNDLAHPLSVNLREGHWALDYILERLNLYNDKPGIRDVQAWLAERFDKIKQLPNFLVPSFFALTIGILYGCCRFKAMLLFPKYIGNANVFVQSLALTSIQMVSEVKSTSIFPNINVPCMCAGLPHFSTNYMRCWGRDVFISFRGLLLVTGRYETAKQHILGFAKTTKHGLIPNLLDAGRNPRYNARDAAWFFLQAIQDYITIVPEGHSILSEKVSRRFPLNDKYITYEDPEAFSYSSSIEEIIYEILSRHAKGIKFREANAGPNLDRVMKDEGFNIEIAVDWSTGLIHGGNQFNCGTWMDKNGESEKAKSKGVPGTPRDGAAVEINGLLKSTLRFVLKLISEGKFKKTDVTRQDGSKISFADWNDLLQENFEKNYYIPLDPKDDNEYNTDSNIVNRRGIYKDLCGSGKPFEDYQLRPNQFIAMAVAPELFSPEHAMSAIKVADENIRGKLGMRTLDPSDYNYRPYYNNSEDSDDFATSKGRNYHQGPEWVWCYGYFMRSFHTFNFLANPLSRNETENRPSSYLMQQIYLRLDDHRKAIIESPWAGLPELTNKDGEFCQDSSPTQAWSSGCILDVLYDVWVAFDSKWYAKQIKQNNPHHHHHH

SEQ ID NO: 336 - protein, 1582 aa, synthetic construct
MIPKTMTSSEVYLLPLSDDGSPQVGGNQYIYLAATSEQPIVVRFAIEGTSSICRHGSLWVNIPEKGAEFRRDHFREFKLDPDFHRTIHISIPIYHPGAFAFYTTYAALPDLSTELAERASFDEEKKTTPRYYIDVAPRLRLDGRPLPLEALSVFSIISKFMGKYPNDWERHLRGISDRGYNMVHFTPLQQRGESNSPYSLYDQLAWDPICFPRGEQDVQDMVHSLKTNHSLLSLTDIVLNHTANNSPWLLDHPDAGYNLKTAPHLESAYLLDSKLLELGDRLGEMGLPTDLKSTDDLDKIMGAIKTECIAGIRLWEYYVLDVENDAANAADSWSRGDITIPDAYAGLNDLAFLKSADLKTQTEFLKKHALHGNDRLGERHRRTIDGKVGAALLTAVLGPASGGAEKDAAHNTLVSMADALNVPFYAEYDAEVADILQQLFNRIKYCRLDDNGPKMGPIGPKSALIETYFTRLPRNEQTAHFDIEELVLVNNGWVWGGNALVDNAGPQSRVYLRREVIVWGDCVKLRYGSGPADSPWLWEHMTQYARTMAKYFAGFRIDNCHSTPMHVAEHILDEARLVNPNLYVVAELFSGSEDTDYIFVKRLGLSSLIREAMQAWSTGEMSRLVHRHGGRPIGSFEVDDITRSDVAKKQQQSALPNANANGHTSEATADGEAEGDELCREIIRRIRVVPVQALFMDCTHDNEAPAQKRDARDTLPNAALVSMCASATGSVMGYDEIYPRLVDLVNETRMYTSESSSRAVKVGSGRNGIGGIKKLLNQIHVLMGKDGYAETHIHHEEEYVTVHRVHPESRKGYFLIAHTAYPGYGNGNGAFNPVHLTGTRANHLGSWMLEVDTSEEATKNALDDTKFLRGLPSRVVDLPGIKIDYNNGDTSIAVPDKFPPGSIALFETWIPAAEHSSGLDTFVTSGAKAAFGELSLVDLNFLLYRCEAEERDASGGNDGVYDLPGYGRLVYAGLQGWYSILKHVVRDNNLAHPLCQNLRDGQWALDYIAGRLERVATKDTHSNLGKPAAWFKERFDAIRPIPAFLHPRYFGLIMKTAYQASWDRAMALMSANVREGQWFVQSLAMVSIQQTGLVQSGSLYPNRLVPSLAAGLPHFAVEWARCWGRDVFISLRGLFLGTGRYAEAKEHILAFASVLKHGMVPNLLGSDGNPRYNARDATWFFLQCIQDYVNRAPDGDELLKTKLKRRFLPNDDTWFDANDPRAYSKESTVEEVIQEALQRHATGMSFREANAGPGLDMQMRDEGFNIDVHVDWSNGFIFGGNQFNCGTWMDKMGESERAGSKGIPGTPRDGAAIEITGLLYSTLKWLAHRHSKGGFAHQGVTRADGSSISFADWAALIQDNFERCYYVPLQPADDAQYDVNPAVVNRRGIYKDLYKSGKEYEDYQLRPNFAIAMTVAADLFDAEHATHALCLADTVLRGPTGMATLDPADLNYRPYYINSEDSTDFATSKGRNYHQGPEWLWPTGFFLRALLKFDLRRRAAQSPDDAEGRTEAFQQVTRRLMGCKEQIQESVWAGLTELTQKDGAYCQDSSPTQAWSAGCLIDLYMDAEEEQARHKTSHVAIR

SEQ ID NO: 337 - protein, 1588 aa, synthetic construct
MIPKTMTSSEVYLLPLSDDGSPQVGGNQYIYLAATSEQPIVVRFAIEGTSSICRHGSLWVNIPEKGAEFRRDHFREFKLDPDFHRTIHISIPIYHPGAFAFYTTYAALPDLSTELAERASFDEEKKTTPRYYIDVAPRLRLDGRPLPLEALSVFSIISKFMGKYPNDWERHLRGISDRGYNMVHFTPLQQRGESNSPYSLYDQLAWDPICFPRGEQDVQDMVHSLKTNHSLLSLTDIVLNHTANNSPWLLDHPDAGYNLKTAPHLESAYLLDSKLLELGDRLGEMGLPTDLKSTDDLDKIMGAIKTECIAGIRLWEYYVLDVENDAANAADSWSRGDITIPDAYAGLNDLAFLKSADLKTQTEFLKKHALHGNDRLGERHRRTIDGKVGAALLTAVLGPASGGAEKDAAHNTLVSMADALNVPFYAEYDAEVADILQQLFNRIKYCRLDDNGPKMGPIGPKSALIETYFTRLPRNEQTAHFDIEELVLVNNGWVWGGNALVDNAGPQSRVYLRREVIVWGDCVKLRYGSGPADSPWLWEHMTQYARTMAKYFAGFRIDNCHSTPMHVAEHILDEARLVNPNLYVVAELFSGSEDTDYIFVKRLGLSSLIREAMQAWSTGEMSRLVHRHGGRPIGSFEVDDITRSDVAKKQQQSALPNANANGHTSEATADGEAEGDELCREIIRRIRVVPVQALFMDCTHDNEAPAQKRDARDTLPNAALVSMCASATGSVMGYDEIYPRLVDLVNETRMYTSESSSRAVKVGSGRNGIGGIKKLLNQIHVLMGKDGYAETHIHHEEEYVTVHRVHPESRKGYFLIAHTAYPGYGNGNGAFNPVHLTGTRANHLGSWMLEVDTSEEATKNALDDTKFLRGLPSRVVDLPGIKIDYNNGDTSIAVPDKFPPGSIALFETWIPAAEHSSGLDTFVTSGAKAAFGELSLVDLNFLLYRCEAEERDASGGNDGVYDLPGYGRLVYAGLQGWYSILKHVVRDNNLAHPLCQNLRDGQWALDYIAGRLERVATKDTHSNLGKPAAWFKERFDAIRPIPAFLHPRYFGLIMKTAYQASWDRAMALMSANVREGQWFVQSLAMVSIQQTGLVQSGSLYPNRLVPSLAAGLPHFAVEWARCWGRDVFISLRGLFLGTGRYAEAKEHILAFASVLKHGMVPNLLGSDGNPRYNARDATWFFLQCIQDYVNRAPDGDELLKTKLKRRFLPNDDTWFDANDPRAYSKESTVEEVIQEALQRHATGMSFREANAGPGLDMQMRDEGFNIDVHVDWSNGFIFGGNQFNCGTWMDKMGESERAGSKGIPGTPRDGAAIEITGLLYSTLKWLAHRHSKGGFAHQGVTRADGSSISFADWAALIQDNFERCYYVPLQPADDAQYDVNPAVVNRRGIYKDLYKSGKEYEDYQLRPNFAIAMTVAADLFDAEHATHALCLADTVLRGPTGMATLDPADLNYRPYYINSEDSTDFATSKGRNYHQGPEWLWPTGFFLRALLKFDLRRRAAQSPDDAEGRTEAFQQVTRRLMGCKEQIQESVWAGLTELTQKDGAYCQDSSPTQAWSAGCLIDLYMDAEEEQARHKTSHVAIRHHHHHH

SEQ ID NO: 338 - protein, 1533 aa, synthetic construct
MTIAAATPKNTILSTGTAGQSSSTLPLVPPLYALLLADDGSPDKSNTSSKLIESPAFCKLLHGNILSRNEHLHIQKREKYVRIPLGNQPCILRFVLRKGSLAATSSTLPRLHTTYPIQGPFDRATFHAVEFDADGICDIPLMTPGAFEYHVTFENNRKSERVGYFVVEPRLYQSSSHKQLPLDAIMIESMIPKWMGPLARWQPHIDLVHQTSYNMIHFAPMQQRGSSDSPYSIRDQLLFADDLFDNPSLLSREDRLKEVRQTIFNIQHEHGILCLSDVVWNHTSHDSVFLQDHPDAGYNLQNSPHLVPAYELDTALLELSKSIDQHGLPQRLETRQDVDNVMSHIKNSIFPQLKLWEYDIIDAEIAIREFSSAITQLSSSHETHKDAYKGAEDVAGQPIKRQAELFGAYAVIEGTHGTRFHRSVDIPRAIAFVQSYAKEDEYKAVFQKLIEQYNLVWYELYDNDVKIALDNIKNRITYLRIDDNGPKWGEISASSPLVESYFTRVNETIQVANNGWIWDADPLEDFAGPESRAYLRRQVICWGDCVKLRYGKSFDDNPWLWDHMRQYTEQVASLFHGIRIDNCHSTPLHVAEYLLDCARRVRPDLYVIAELFTGSPEMDERFVSRLGIHALIREAMQAWDPHEMSRLVHRHGGKPVGSMDQFIRWKRISKDGNEYMLIPIEHGSMPRALFMDCTHDNKTPFQMRTAEDALANSALVAFADCATGSVKGYDEIYPRLLDLVGEKRHYNPNVDASTTGIMRIKEQLQHLHMKMALEGYREVHVHHEGDYVLVHRQHPRTHDGYFLVARTAFPDKHGSIDPIRLHRTRLSFMFGASLHVDTAAAAPLDTKHLVGLPSVLADLPEPPIHSRENGFVEIELPSDFTPGSILVFQTFAQIHDTSNVTQLVTSMPESVLESLDVLDCNVVLYRCQGEEFDATNGGGVYEVPGWGQMTYAGLEGFMAVLRPIIRDNDLGHPFCDNLRKGHWAMDYIQNRLEVYQKNFEHHHLKPLIQWFSSRFGVIRGLPDFLVPKYFSMAVHTAYEHVRDHALQCMAPFVHASLDPFIHQLALCSVQMLGVVPSTSLEPIKITPTLAAGLPHFTHGCMRTWGRDVFISLRGLLMVTGQWDAARQHILAFASTLRNGLLPNLLDAGRNPRYNARDAVWWFMQSVQDYCKLAPDGTDILSAKVSRRFPRDYEWVSHDHGYDWDCTLAELIQEIMQQHAKGIHFREHNAGPNIDQQMKDEGFNIDIEVDWESGGVLIGGNIWNCGTWMDKMGESEKAGNKGYPGTPRDGAPIEITGLLKSALRWLSSLCNTDVFPWKGIDRANGKTITYSEWNDLLQSNFERIYFVPREPDQDTNYAVDPSIVHRRGIYKDAYGATEPYTEYQFRPNLCVAMVVAPELFNRQHAKQCLQLMHNVLLAPLGMRTLDPADMRYRPYYINGEDSEDFDTAKGRNYHQGPEWIWPIGYYLRAAHQFGALSSLAISRVLHAHRQEIETSTWAGLPELTNKDGETCWDSCFSQAWSAATILDLLYDMQQ

SEQ ID NO: 339 - protein, 1539 aa, synthetic construct
MTIAAATPKNTILSTGTAGQSSSTLPLVPPLYALLLADDGSPDKSNTSSKLIESPAFCKLLHGNILSRNEHLHIQKREKYVRIPLGNQPCILRFVLRKGSLAATSSTLPRLHTTYPIQGPFDRATFHAVEFDADGICDIPLMTPGAFEYHVTFENNRKSERVGYFVVEPRLYQSSSHKQLPLDAIMIESMIPKWMGPLARWQPHIDLVHQTSYNMIHFAPMQQRGSSDSPYSIRDQLLFADDLFDNPSLLSREDRLKEVRQTIFNIQHEHGILCLSDVVWNHTSHDSVFLQDHPDAGYNLQNSPHLVPAYELDTALLELSKSIDQHGLPQRLETRQDVDNVMSHIKNSIFPQLKLWEYDIIDAEIAIREFSSAITQLSSSHETHKDAYKGAEDVAGQPIKRQAELFGAYAVIEGTHGTRFHRSVDIPRAIAFVQSYAKEDEYKAVFQKLIEQYNLVWYELYDNDVKIALDNIKNRITYLRIDDNGPKWGEISASSPLVESYFTRVNETIQVANNGWIWDADPLEDFAGPESRAYLRRQVICWGDCVKLRYGKSFDDNPWLWDHMRQYTEQVASLFHGIRIDNCHSTPLHVAEYLLDCARRVRPDLYVIAELFTGSPEMDERFVSRLGIHALIREAMQAWDPHEMSRLVHRHGGKPVGSMDQFIRWKRISKDGNEYMLIPIEHGSMPRALFMDCTHDNKTPFQMRTAEDALANSALVAFADCATGSVKGYDEIYPRLLDLVGEKRHYNPNVDASTTGIMRIKEQLQHLHMKMALEGYREVHVHHEGDYVLVHRQHPRTHDGYFLVARTAFPDKHGSIDPIRLHRTRLSFMFGASLHVDTAAAAPLDTKHLVGLPSVLADLPEPPIHSRENGFVEIELPSDFTPGSILVFQTFAQIHDTSNVTQLVTSMPESVLESLDVLDCNVVLYRCQGEEFDATNGGGVYEVPGWGQMTYAGLEGFMAVLRPIIRDNDLGHPFCDNLRKGHWAMDYIQNRLEVYQKNFEHHHLKPLIQWFSSRFGVIRGLPDFLVPKYFSMAVHTAYEHVRDHALQCMAPFVHASLDPFIHQLALCSVQMLGVVPSTSLEPIKITPTLAAGLPHFTHGCMRTWGRDVFISLRGLLMVTGQWDAARQHILAFASTLRNGLLPNLLDAGRNPRYNARDAVWWFMQSVQDYCKLAPDGTDILSAKVSRRFPRDYEWVSHDHGYDWDCTLAELIQEIMQQHAKGIHFREHNAGPNIDQQMKDEGFNIDIEVDWESGGVLIGGNIWNCGTWMDKMGESEKAGNKGYPGTPRDGAPIEITGLLKSALRWLSSLCNTDVFPWKGIDRANGKTITYSEWNDLLQSNFERIYFVPREPDQDTNYAVDPSIVHRRGIYKDAYGATEPYTEYQFRPNLCVAMVVAPELFNRQHAKQCLQLMHNVLLAPLGMRTLDPADMRYRPYYINGEDSEDFDTAKGRNYHQGPEWIWPIGYYLRAAHQFGALSSLAISRVLHAHRQEIETSTWAGLPELTNKDGETCWDSCFSQAWSAATILDLLYDMQQHHHHHH

SEQ ID NO: 340 - protein, 1490 aa, synthetic construct
MTKIHLLELNDDGTPCVGTALVRLPAPFEPYTLRFVLYSTNQLDSGATLKINVPAAGQVFDRQKFLTKSLKAEAGTDACVDVEIDQPGAFAWRIEYLNEDAAAITTAEYYIVVDPALTINGTQISLDALAIQSVVPKWMGKLTQWSTYLEYMSSVKKYNMLHFCPLQQRGASDSPYSLFDQHKFSDDLFDNVSLSLKDRTAQMEAMVNDMEHKYGLLSLTDVVWNHTASDSDWLQDHPEAGFNLHNSPHLCAAFALDTALLELSSNLSKLGLPTTLKSTQDLLAIMEAIKSRVLGTIKLWEFYVIDTQKALSTMVFTQQPAPVGELNSQSSLESIADFCLSQGVVQNHQFLGDRFSKIVDPAKLGAIINALCCAKTDKLAFASRILDEINLRFYQEHNADQDAFMEQLYNRIKYVRLDSHGPKMGPITSMAPLIETYFTRLPSNDRTRKHPSGSLALANNGWMWAANPLENFAGPQSKAYLRREVIVWGDCVKLRYGDCPADNPYLWEHMRLYTSQMASIFHGFRIDNCHSTPIPVARYLLDEARKSRPNLYVVAELFTGSEDMDTTFVQKLGICSLLREAMQAWSPDELSRLVHKYGPSPIGSIPNQLGNVDSSQLIKPAPIHALFFDCTHDNETPVQKRSAEDTLPNAALVAMTASAIGSVFGYDEVYPHILDLVSEKRHYSQRENGLGRAKGVLNAVHIEMGLKNYSEAHVHHEGAYITVHRVSPETKEGYFLIAHTAFHGGQELGAISPIRLSGTSLELVYAATLSVTPAQKDASEYTELDGVPASVVMLDFPVETTDDDDTILTVPGRFPPGSIALFRTKLRLPKLPESPELSWEKLEDFLMMGASEAVASCSLIDLNVLLYRCNAEEQDTVAEGAYNIPNYGQLVYAGLQGWMAPISAVTRSNDLGHPLCDHLRNGLWALDYIVAREQRYIRNGYDALSPSSRWLSTRFDYIKRLPNFLVPKAFVLVIKALYCAARERSLCLLSKQIRRGTKFTQDLALCSLQMQGAVKSASIDPAKSIPCMAAGLPHFSTSWARCWGRDIFIAMRGLLLVTERYAEAESHILAFATTLKHGMIPNLLDSLRTPRYNSRDSIWFYMQAIQDYVTMVPNGAEILKKSVKRRFPLNDEYVEWDDQRAYSYQTSVTDVIQETLARHAQGLHFREANAGPKLDMQMSNEGFNIDVEVDWSTGFLHGGNQHNCGTWMDKMGESERAGSKGVPGTPRDGAAVEITGLLKSTLRWVNQLHQQGLYPYTCVRATIDGKETDMTFVRWEALIQSNFERCYYIPKQSSDDAQYEVDAAIVNRRGIYKDLYKSGKAYEDYQLRAQVPMAMTVAPELFETEHALSHLETADHFLRGPLGMATLDPIDNNYRPYYNNTEDSTDFHTAKGRNYHQGPEWVFPVGYFLRAMLHFQIQDGRTNSDEISRQIYRRLSAHRTEIMSTPWTGLTELTNKAGALCGDSSPTQAWSSGTLLDVIHDMQELRVKQL

SEQ ID NO: 341 - protein, 1496 aa, synthetic construct
MTKIHLLELNDDGTPCVGTALVRLPAPFEPYTLRFVLYSTNQLDSGATLKINVPAAGQVFDRQKFLTKSLKAEAGTDACVDVEIDQPGAFAWRIEYLNEDAAAITTAEYYIVVDPALTINGTQISLDALAIQSVVPKWMGKLTQWSTYLEYMSSVKKYNMLHFCPLQQRGASDSPYSLFDQHKFSDDLFDNVSLSLKDRTAQMEAMVNDMEHKYGLLSLTDVVWNHTASDSDWLQDHPEAGFNLHNSPHLCAAFALDTALLELSSNLSKLGLPTTLKSTQDLLAIMEAIKSRVLGTIKLWEFYVIDTQKALSTMVFTQQPAPVGELNSQSSLESIADFCLSQGVVQNHQFLGDRFSKIVDPAKLGAIINALCCAKTDKLAFASRILDEINLRFYQEHNADQDAFMEQLYNRIKYVRLDSHGPKMGPITSMAPLIETYFTRLPSNDRTRKHPSGSLALANNGWMWAANPLENFAGPQSKAYLRREVIVWGDCVKLRYGDCPADNPYLWEHMRLYTSQMASIFHGFRIDNCHSTPIPVARYLLDEARKSRPNLYVVAELFTGSEDMDTTFVQKLGICSLLREAMQAWSPDELSRLVHKYGPSPIGSIPNQLGNVDSSQLIKPAPIHALFFDCTHDNETPVQKRSAEDTLPNAALVAMTASAIGSVFGYDEVYPHILDLVSEKRHYSQRENGLGRAKGVLNAVHIEMGLKNYSEAHVHHEGAYITVHRVSPETKEGYFLIAHTAFHGGQELGAISPIRLSGTSLELVYAATLSVTPAQKDASEYTELDGVPASVVMLDFPVETTDDDDTILTVPGRFPPGSIALFRTKLRLPKLPESPELSWEKLEDFLMMGASEAVASCSLIDLNVLLYRCNAEEQDTVAEGAYNIPNYGQLVYAGLQGWMAPISAVTRSNDLGHPLCDHLRNGLWALDYIVAREQRYIRNGYDALSPSSRWLSTRFDYIKRLPNFLVPKAFVLVIKALYCAARERSLCLLSKQIRRGTKFTQDLALCSLQMQGAVKSASIDPAKSIPCMAAGLPHFSTSWARCWGRDIFIAMRGLLLVTERYAEAESHILAFATTLKHGMIPNLLDSLRTPRYNSRDSIWFYMQAIQDYVTMVPNGAEILKKSVKRRFPLNDEYVEWDDQRAYSYQTSVTDVIQETLARHAQGLHFREANAGPKLDMQMSNEGFNIDVEVDWSTGFLHGGNQHNCGTWMDKMGESERAGSKGVPGTPRDGAAVEITGLLKSTLRWVNQLHQQGLYPYTCVRATIDGKETDMTFVRWEALIQSNFERCYYIPKQSSDDAQYEVDAAIVNRRGIYKDLYKSGKAYEDYQLRAQVPMAMTVAPELFETEHALSHLETADHFLRGPLGMATLDPIDNNYRPYYNNTEDSTDFHTAKGRNYHQGPEWVFPVGYFLRAMLHFQIQDGRTNSDEISRQIYRRLSAHRTEIMSTPWTGLTELTNKAGALCGDSSPTQAWSSGTLLDVIHDMQELRVKQLHHHHHH

SEQ ID NO: 342 - protein, 1475 aa, synthetic construct
MSPPLVYMLPLTDTGAPDIAGDHIGLPPPREPYTLRIAIEGASSITRNGSLWTNVPAKGDNFGRNKFREFKLTPSFSRTLHIDVPIALPGTFCYYITYDPLPSLKELSSPTNPSGGAKTGTHWFTVAARLSVNGEPLALDSLSCFSVVSKWLGPVEKWDGYMRYIAEKGYNMVHFTPMMHRGESNSPYSIYDQLAFDPEAFPNGTDDVALIVDRMEKRHGLLSLTDVVWNHTAHNSLWLQSHPESGYNLVTAPWLEAAFELDTALLDFGNHLEALGFPTELETQEDLLKIMDGVKTHVIGALRLWEFYVVNVEAATDAVIEAYISGKSDSKLIDTAEYKSGLSLHDMTQFLIKNGLKGADILGSRYSRTIDPGVGAAFLALECGKPDPKNAKGVEEAKQALIRVYNRITYLRLDSNGPKMGKITKESPIIETYFTRLPKNEITAQHDPKSLALANNGWIWSNVADFAGPKSRSYLRREVIVWGDCVKLRFGDKPEDNPFLWQFMVEYTKLMARCFHGFRIDNCHSTPIHVGQYLLDIAREVREDLYVVAELFTGSEHQDRRFVEHLGLGSLVREAMVAWGPGELSRLVHRHGGKPIGSLRQELVVRAVTASPIHALFMDCTHDNETPTQKRTPQDTLPNGALVAMCDCGIGSVMGYDEIYPKLLNLVTEPRIYYPPPEKIDETTPGIGKVKGLLNRIHTEMGKDGFTEMHVHHEGEYITVHRVHPKSHKGYFLIAHTAFGPGDHRGDFNPITVTGTKVKVIGSWTLVADGSDERKEKVLSQPDYLTGLPAKLVEVESPKIGENGNTTTITVPEKFPPGSIALLETWIPGTDGESLAQFVTSGAEEAFANTSLVDLNFVLYRCESEERDSSDDRDGCYNIPGYGALVYAGLQGWWSVLKDVIRNNDLAHPICQHLRDGQWALDYIVGRLEKLEARGNKGLAEPTEWLKERFDRIRSVPGFLLPRYFALVMQTAYTTAVERATSFFTEDIREGTAFLKELSLVSLQMTGLMDSTSLWPGKKVACMAAGLPHFSYDYMRCWGRDVFISIRGLYLATGRYEDAKEHILAFGSVLKHGMIPNLLDGGRKPRYNARDSIWFYLQAVQDYTGIVPNGIELLREKVKRRFLPFDDTWFNYDDPRAYSRESTIAEIIHEALERHALGFEFLEANAGPGLDNQMKWEGFTVGVKPDWTTGIIKGGSQFNCGTWMDKMGESERAGNKGVPGTPRDGAAIEITGLLYSTLKWVAELRKKKLYKWDSVKTSQGKGITFDMWADLLKKNFEKLYYVPEDPGEDSKYSIKPELIGRRGIYKDVWGGGKEYEDYQFRPNFAIAMTVAPDLFVPKHAIRCLEIADEVLRGPTGMATLDPIDKDYRPYYRNSEDSDDFATSKGRNYHQGPEWLWPTGFYLRALLTFSPPNIETLQQINLRMDGCRKAINESPWAGLTELTNKNGEICYDSCPTQAWSASCLIDLCYDAAKIKL

SEQ ID NO: 343 - protein, 1481 aa, synthetic construct
MSPPLVYMLPLTDTGAPDIAGDHIGLPPPREPYTLRIAIEGASSITRNGSLWTNVPAKGDNFGRNKFREFKLTPSFSRTLHIDVPIALPGTFCYYITYDPLPSLKELSSPTNPSGGAKTGTHWFTVAARLSVNGEPLALDSLSCFSVVSKWLGPVEKWDGYMRYIAEKGYNMVHFTPMMHRGESNSPYSIYDQLAFDPEAFPNGTDDVALIVDRMEKRHGLLSLTDVVWNHTAHNSLWLQSHPESGYNLVTAPWLEAAFELDTALLDFGNHLEALGFPTELETQEDLLKIMDGVKTHVIGALRLWEFYVVNVEAATDAVIEAYISGKSDSKLIDTAEYKSGLSLHDMTQFLIKNGLKGADILGSRYSRTIDPGVGAAFLALECGKPDPKNAKGVEEAKQALIRVYNRITYLRLDSNGPKMGKITKESPIIETYFTRLPKNEITAQHDPKSLALANNGWIWSNVADFAGPKSRSYLRREVIVWGDCVKLRFGDKPEDNPFLWQFMVEYTKLMARCFHGFRIDNCHSTPIHVGQYLLDIAREVREDLYVVAELFTGSEHQDRRFVEHLGLGSLVREAMVAWGPGELSRLVHRHGGKPIGSLRQELVVRAVTASPIHALFMDCTHDNETPTQKRTPQDTLPNGALVAMCDCGIGSVMGYDEIYPKLLNLVTEPRIYYPPPEKIDETTPGIGKVKGLLNRIHTEMGKDGFTEMHVHHEGEYITVHRVHPKSHKGYFLIAHTAFGPGDHRGDFNPITVTGTKVKVIGSWTLVADGSDERKEKVLSQPDYLTGLPAKLVEVESPKIGENGNTTTITVPEKFPPGSIALLETWIPGTDGESLAQFVTSGAEEAFANTSLVDLNFVLYRCESEERDSSDDRDGCYNIPGYGALVYAGLQGWWSVLKDVIRNNDLAHPICQHLRDGQWALDYIVGRLEKLEARGNKGLAEPTEWLKERFDRIRSVPGFLLPRYFALVMQTAYTTAVERATSFFTEDIREGTAFLKELSLVSLQMTGLMDSTSLWPGKKVACMAAGLPHFSYDYMRCWGRDVFISIRGLYLATGRYEDAKEHILAFGSVLKHGMIPNLLDGGRKPRYNARDSIWFYLQAVQDYTGIVPNGIELLREKVKRRFLPFDDTWFNYDDPRAYSRESTIAEIIHEALERHALGFEFLEANAGPGLDNQMKWEGFTVGVKPDWTTGIIKGGSQFNCGTWMDKMGESERAGNKGVPGTPRDGAAIEITGLLYSTLKWVAELRKKKLYKWDSVKTSQGKGITFDMWADLLKKNFEKLYYVPEDPGEDSKYSIKPELIGRRGIYKDVWGGGKEYEDYQFRPNFAIAMTVAPDLFVPKHAIRCLEIADEVLRGPTGMATLDPIDKDYRPYYRNSEDSDDFATSKGRNYHQGPEWLWPTGFYLRALLTFSPPNIETLQQINLRMDGCRKAINESPWAGLTELTNKNGEICYDSCPTQAWSASCLIDLCYDAAKIKLHHHHHH

SEQ ID NO: 344 - protein, 1563 aa, synthetic construct
MTRVVLLRLDDKGEPLVNGHGVLSFPAVPPEPTRKSDDPLFCLRLHIQAGSRIANNGKVWTDVPPSGSIAFSREKFYGQSILGSFDKDSTVDINVYWPGAYCYYLSYNSLDDDDNETLKTTLKFHFVVPPSLFISQKYLPLNSISMQSVISKWIGTGRDDFDRLFSEIQRKGYNMIHFTPLQARGESDSPYSICDQLEFDPALFPGGVDDVSRMIAELETRHGILSMTDVVFNHTANNSPWLREHPEAGYNLETAPHLEAAMELDALLLHFSRYMSWHGCPTNIRTTADLLKVMDGIKIHVLGQLRLWQYYVIDVKGELARLKGEWDASRNGKGGKVDPAIFSDFPPDARSDLRVAARYIAATCAVKPFGLGARYENALDTVKLAAVLEKIFDKKLVDLDFPDLEKRTHAILDEVNLPLYKEYDEDNEDILENLYNRINYQRLDSHGPQLGEVTKDSPLTEPYFTRFTDVNGRKWALANNGWIWDGNPLVDFASSSSKCYMRREVIVWGDCVKLRYGSGPQDSPYLWDRMTKYAQLCAKVFHGFRIDNCHSTPIHVGEALLDAARAIRPNLYVVAELFTGNEDLDTHFVERLGISSLIREAMQAYSVGELSRLVHRHGGRPIGSFRWLPLDVLAYPADKLEFAERQAEEIRRRSEIPVPELVTAAPTHALFMDCTHDNEMPNDKRTVEDTLPNAALVAFCACATGTTMGYDECYPHLLDLVKGKQMYTYGPGIGIGDVKAKLNSLRRVLAEQSLSDPEANEMHVHHEGQYITIHRSNAQTGEGYFLIARTKFCPDGEQTLSPIVLHGTLVKNEFAYALERLSAFTRVRSGETDTEEVDPLSKAAAPSDFITPVPVKLRTIEPADCLYDPVEHSTTVILPKKFPQGAILVLSTRIPNCDAELDKYVRTGAIEAAEELTLVDLNALLYRCESEERDASGGVDGVYDIPDFGRLVYAGLQGWVSVLRGMVASNDLAHPLAKHLRNGHWALDYIPARIAKYEAAASYAGDDDRAAAIERFRTWIESRFARVKEVPYFLVPRFFALVVGVAYEALRFRALALMSPPVKRSTVFVQSLALVSVQMAGLTRTAPLSPFSTNEPSLAAGLPHFSFDFMRCWGRDVFISLRGLLLATGRFDVAKSHILNFAMTLKHGLIPNLLGAGKEPRYNARDAVWWFLQAIQDLYHSAPIGEGDELLNARVRRRFPLDDTWVPWDDARAFAVESTLVQIIYEILSRHAAGIKFREAHAGPQIDSQMRDEGFNVEIHVDWTTGLVSGGNQFNCGTWMDKMGESVRAGNKGIPGTPRDGADVEINGLLKSALRFVIELNEKGLFPYTSITTQDGKDVTFVQWNQLLQANFEKCFYIPENPSEDKDFVVDSNIVNRRGIYKDIFGSGKPYEDYQLRGNFPIAIVVAPELFKPERALRAIDLADRVLRGPVGLKTLDPSDLNYRPYYNNSEDSNDFATSKGRNYHQGPEWVWIYGYFLRAYRMLHEKYDDRCKNESYLDQLMARRLSGNLKWLKESQWAGLAELTNKDGEFCSDSCMTQAWSASCLVDVYMDYWRGESKEHLL

SEQ ID NO: 345 - protein, 1569 aa, synthetic construct
MTRVVLLRLDDKGEPLVNGHGVLSFPAVPPEPTRKSDDPLFCLRLHIQAGSRIANNGKVWTDVPPSGSIAFSREKFYGQSILGSFDKDSTVDINVYWPGAYCYYLSYNSLDDDDNETLKTTLKFHFVVPPSLFISQKYLPLNSISMQSVISKWIGTGRDDFDRLFSEIQRKGYNMIHFTPLQARGESDSPYSICDQLEFDPALFPGGVDDVSRMIAELETRHGILSMTDVVFNHTANNSPWLREHPEAGYNLETAPHLEAAMELDALLLHFSRYMSWHGCPTNIRTTADLLKVMDGIKIHVLGQLRLWQYYVIDVKGELARLKGEWDASRNGKGGKVDPAIFSDFPPDARSDLRVAARYIAATCAVKPFGLGARYENALDTVKLAAVLEKIFDKKLVDLDFPDLEKRTHAILDEVNLPLYKEYDEDNEDILENLYNRINYQRLDSHGPQLGEVTKDSPLTEPYFTRFTDVNGRKWALANNGWIWDGNPLVDFASSSSKCYMRREVIVWGDCVKLRYGSGPQDSPYLWDRMTKYAQLCAKVFHGFRIDNCHSTPIHVGEALLDAARAIRPNLYVVAELFTGNEDLDTHFVERLGISSLIREAMQAYSVGELSRLVHRHGGRPIGSFRWLPLDVLAYPADKLEFAERQAEEIRRRSEIPVPELVTAAPTHALFMDCTHDNEMPNDKRTVEDTLPNAALVAFCACATGTTMGYDECYPHLLDLVKGKQMYTYGPGIGIGDVKAKLNSLRRVLAEQSLSDPEANEMHVHHEGQYITIHRSNAQTGEGYFLIARTKFCPDGEQTLSPIVLHGTLVKNEFAYALERLSAFTRVRSGETDTEEVDPLSKAAAPSDFITPVPVKLRTIEPADCLYDPVEHSTTVILPKKFPQGAILVLSTRIPNCDAELDKYVRTGAIEAAEELTLVDLNALLYRCESEERDASGGVDGVYDIPDFGRLVYAGLQGWVSVLRGMVASNDLAHPLAKHLRNGHWALDYIPARIAKYEAAASYAGDDDRAAAIERFRTWIESRFARVKEVPYFLVPRFFALVVGVAYEALRFRALALMSPPVKRSTVFVQSLALVSVQMAGLTRTAPLSPFSTNEPSLAAGLPHFSFDFMRCWGRDVFISLRGLLLATGRFDVAKSHILNFAMTLKHGLIPNLLGAGKEPRYNARDAVWWFLQAIQDLYHSAPIGEGDELLNARVRRRFPLDDTWVPWDDARAFAVESTLVQIIYEILSRHAAGIKFREAHAGPQIDSQMRDEGFNVEIHVDWTTGLVSGGNQFNCGTWMDKMGESVRAGNKGIPGTPRDGADVEINGLLKSALRFVIELNEKGLFPYTSITTQDGKDVTFVQWNQLLQANFEKCFYIPENPSEDKDFVVDSNIVNRRGIYKDIFGSGKPYEDYQLRGNFPIAIVVAPELFKPERALRAIDLADRVLRGPVGLKTLDPSDLNYRPYYNNSEDSNDFATSKGRNYHQGPEWVWIYGYFLRAYRMLHEKYDDRCKNESYLDQLMARRLSGNLKWLKESQWAGLAELTNKDGEFCSDSCMTQAWSASCLVDVYMDYWRGESKEHLLHHHHHH

SEQ ID NO: 346 - protein, 1551 aa, synthetic construct
MASQVFVLPLSDMGEPQPGPGGDFINLPIPYESYKLRFVIFSTSSVTNESSLWCNVPQDPSAPFERNKFYEYPIRSQFNKDTYVDVDIHTPGTYSFYITYLPISQNYFERYEEKHANGFNDEMMASQNPVERSLFKETYIAMDVKGHLASTRKYYFSVSAGFTLNNNPLPLNALSIQTVISKWMGEYSTWDKKLAQIKGKGYNMIHFTPLQERGDSDSPYSIFDQLKWDPKCFKNGEDDVKDLVETMEKKHCLLSMTDIVLNHTANNSEWLKHHPDAGYSVYTAPHLRPALELDDELTAYSGRLKELRLPTVLETEDDVDNVILGINRNVLQKIRLWEFYVVDVKSTIKRVREVVENDERFNAIYPTRVPPNLEDDLTDLAKYVVAEAGSGFYEFGPRFIKAIDADYFVSILKALFPDPNVEPSIIVEKATDILNEINSPLYKDYDFDKAEIISNLRGRLKYLRLEDGGPKLGEITAEKPIHESYFTKVKTEPKGEVVSLVNNGFIWNGDPMIDFASSNSKAYLRREVISWGDCVKLRYGSKPADSPFLWEHMTKYTQTMAKYFHGFRLDNCHSTPIHVAEYLLDKARLIRSNLYVAAELFTGSESKDRVFVRRLGITSLIREAMQAWSPGELSRLVHKHGGRPIGSFSKQPLVKHAQLQENPQNVHFIRTTPIHALFMDCTHDNEMPFQKRTVEDTISTAALVSMCACAVGSTMGFDECYPHHLNVVHEKRPYTFGSGISEVKATLLDIHEDMGCKNAEEMFVHHEGQYITVHRVNPKEGTGWFLIARTKFSEDDDQKSKSPLPNGHPYAINDITLPGTQAVVHKTAALRVTGEYEDDENQLNGIPTEIVHVEEPEITYNENTENTTIKFKDAIPQGSFVLLRTFVTAIEYGLDKFVRSGADEALEKVDLLDLNVIMYRCEAEERDASDGKDGVYVVPNYGPLTYAGLHGWIHPVREMVALNDTAHPIADNLREGNWAMDFAISRLFKYLKHYPNLKSFIDWLESRFNRVRAIPQVLVPRYFALVIFTAYTACFRRAMNLMPGSLKRSTMFLNNLALVSVQMVGKVPSASVFPFETVGSMAAGLPHFSSGYMRCWGRDVFLSFKGLLLSTGRFEEAKNHILGFAATLKHGLVPNLLDSGRYPRYNARDATWFFLQSIQDYCKAVPHGIKILDEKVKRRFPLDDTFVELGDPRAFSEESTIREVIYEIFARHAKGIQFREANAGPDIDQQMKDEGFNQNIYVDWKNGIIFGGNQWNCGTWMDKMGESEKAGNKGYPGTPRDGAAIEITGLLKSALRWVNELRVEEEFPWNTVTNQDGDEVSFIEWERKLQRSFEKAYYIPKDPKKDILFDVDSKIVHRRGIYKDLYRSGKPYEDYQLRPNFAIAMTVAPELFDVDRAMGALAAADTIILGPVGMRTLDPDDYNYRPYYENSIDNDDFATSKGRNYHQGPEWVWLRGYYLRAMLQFDVLRKKRYGGDMKETFQQLHLRMIELSLWIRQSAWGGLTELTNKDGAICSDSSPTQAWSAATIIDLFEDSKHYLEYSKAHHKKPSY

SEQ ID NO: 347 - protein, 1557 aa, synthetic construct
MASQVFVLPLSDMGEPQPGPGGDFINLPIPYESYKLRFVIFSTSSVTNESSLWCNVPQDPSAPFERNKFYEYPIRSQFNKDTYVDVDIHTPGTYSFYITYLPISQNYFERYEEKHANGFNDEMMASQNPVERSLFKETYIAMDVKGHLASTRKYYFSVSAGFTLNNNPLPLNALSIQTVISKWMGEYSTWDKKLAQIKGKGYNMIHFTPLQERGDSDSPYSIFDQLKWDPKCFKNGEDDVKDLVETMEKKHCLLSMTDIVLNHTANNSEWLKHHPDAGYSVYTAPHLRPALELDDELTAYSGRLKELRLPTVLETEDDVDNVILGINRNVLQKIRLWEFYVVDVKSTIKRVREVVENDERFNAIYPTRVPPNLEDDLTDLAKYVVAEAGSGFYEFGPRFIKAIDADYFVSILKALFPDPNVEPSIIVEKATDILNEINSPLYKDYDFDKAEIISNLRGRLKYLRLEDGGPKLGEITAEKPIHESYFTKVKTEPKGEVVSLVNNGFIWNGDPMIDFASSNSKAYLRREVISWGDCVKLRYGSKPADSPFLWEHMTKYTQTMAKYFHGFRLDNCHSTPIHVAEYLLDKARLIRSNLYVAAELFTGSESKDRVFVRRLGITSLIREAMQAWSPGELSRLVHKHGGRPIGSFSKQPLVKHAQLQENPQNVHFIRTTPIHALFMDCTHDNEMPFQKRTVEDTISTAALVSMCACAVGSTMGFDECYPHHLNVVHEKRPYTFGSGISEVKATLLDIHEDMGCKNAEEMFVHHEGQYITVHRVNPKEGTGWFLIARTKFSEDDDQKSKSPLPNGHPYAINDITLPGTQAVVHKTAALRVTGEYEDDENQLNGIPTEIVHVEEPEITYNENTENTTIKFKDAIPQGSFVLLRTFVTAIEYGLDKFVRSGADEALEKVDLLDLNVIMYRCEAEERDASDGKDGVYVVPNYGPLTYAGLHGWIHPVREMVALNDTAHPIADNLREGNWAMDFAISRLFKYLKHYPNLKSFIDWLESRFNRVRAIPQVLVPRYFALVIFTAYTACFRRAMNLMPGSLKRSTMFLNNLALVSVQMVGKVPSASVFPFETVGSMAAGLPHFSSGYMRCWGRDVFLSFKGLLLSTGRFEEAKNHILGFAATLKHGLVPNLLDSGRYPRYNARDATWFFLQSIQDYCKAVPHGIKILDEKVKRRFPLDDTFVELGDPRAFSEESTIREVIYEIFARHAKGIQFREANAGPDIDQQMKDEGFNQNIYVDWKNGIIFGGNQWNCGTWMDKMGESEKAGNKGYPGTPRDGAAIEITGLLKSALRWVNELRVEEEFPWNTVTNQDGDEVSFIEWERKLQRSFEKAYYIPKDPKKDILFDVDSKIVHRRGIYKDLYRSGKPYEDYQLRPNFAIAMTVAPELFDVDRAMGALAAADTIILGPVGMRTLDPDDYNYRPYYENSIDNDDFATSKGRNYHQGPEWVWLRGYYLRAMLQFDVLRKKRYGGDMKETFQQLHLRMIELSLWIRQSAWGGLTELTNKDGAICSDSSPTQAWSAATIIDLFEDSKHYLEYSKAHHKKPSYHHHHHH

SEQ ID NO: 348 - protein, 1753 aa, synthetic construct
MPYTPVDIRRNRLKASAAPKSFKAKPQNDDVLRIYTLELEDDGSPEYSKRYIRLAPTQKDVFVIRFKVTAGMLASNNGVLYTNYPISGCFDREKFHEVRFSSDFSKDTYCDVTVTTPGAYEYYVEYEPINQENSDNQPEPKRSSRSGYFVVEPRLYIDSPVKPSEPSAEKKEVQEHAPAKVLLPLDGLVIESAVGKWLGPLDTWDAHLQHMKAAGYNMIHFVPLQVRGASNSPYSIHDQTAFSDDLFNEQDVKKSSEEKAQLVNKQLHRIQDEYGIMCLSDVVWNHTSFDSVWLQDHPESGYNLDNSPHLQAAFELDTALINLVDDFSKYSLPTTLKSGQDLDAVMNVIKNNVFPDLKLWEYKVIDVVSEKKKLHEALLAAPKETAQSQVFENANLSGLASDQQAQLLAEHGIADRQSGSRYGKTVDIDVAVAFIKALGGVASGEQVSEDVAAKLVDIYGKVLDVYNLPFYKEYDEDVRIALENTKNRIEYTRLADHGPKWGEITKEKPIVDNYFTRLPKNDKTKGHSEGSLALANNGWIWNADPMQDFAGPQSAAYLRREVICWGDCVKLRYGSGADDNPYLWKHMKDYTEQTAKYFQGIRIDNCHSTPIHVAQYLLDAARKIRPDLYVVAELFTGSEDSDVTFVSKLGINSLIREAMQAWDAAELSRLVHRHGGKAIGSMDQALTTEQINYSLNGKEEEPALLVPVLQGSVPHALFMDCTHDNETPNEKRTPQDTLPNAALVAFSGSAIGSVKGYDDIYPRLLDIVNETRLYDLPKPDANVGINKVKPVLQKLHTEMIMDGYIECHVHHEGDYIIVHRQHPELHTGYLLVAHTAFPGRQNNRGSISPIKLRGTDVDIMFSATLDIESRENLSDEKTLRGLKSKIVSLSAPTIKSLSDNKGRYTEITVPASFPSGSIMVLKTWIQDNPAESYDLISSCSDDVFKNMTSLDMNVVLHRCEGEERDATGEGVYNIPGFGGLPYAGLEGFVSVLKPIIAHNDLGHPFCAHLREGTWAMDFIVDRLNRYIGNYPNLKRLKDWFASRFSEVKGLPDFLLPKYFALIIRTAYLKARAHAVSLMSPLVREGDRFIQSLALCSVQMYGIVPSSGLHPTEVTPSMAAGLPHFTHSYMRTWGRDVFIALRGLFLTTGNYDAAKRHIVSFASSLKHGLIPNLLDAVRHPRYNSRDSVWWFLQAVQDYYNMAPDGKSILQEKVSRRFPKNDTFVPPEEGFKYSCTVAEIIQEIFERHAAGIHFREHNAGEQLDRQMLDEGFNIDIDVDWESGTLIGGNRFNCGTWQDKMGESVKAGNKGLPATPRDGAPIEITGLLKSTLRWVVELHERGEFQWTSAKDKSGNERSYKDWNDLLQTNFERVYYVPLDEKEDSQYDVLSQIVNRRGIYKDVYKSKEPYTDYQFRSNVPVAMVVAPELFTPAKAMGSLELARSNLLGPLGMATLDPSDNEYHPDYHNSDDSDNKAVAKGWNYHQGPEWVWQTGYFLRAYLYFSLKTGEDQSKREKVMDIQRILLAHKREIESSPWAGLPELTNRNGNPCWDSCPTQACYDDEVVLIQSQNTSFNHRSPATSDQESAAINFFLFCANLISEEPMNNDQVIPLETHKLGLELRPHPQRGRGVFATCALKARTLVEISPILLVNHDEYSAHGQYTILDHYTYRWEGGYALALGLGSMFNHAKNPNVGFVRDIPNAVIRYFTLRAIEPAEELCISYGDHLWFEDTDAKSEASIDSSEDEWFGKIMVDDEE

SEQ ID NO: 349 - protein, 1759 aa, synthetic construct
MPYTPVDIRRNRLKASAAPKSFKAKPQNDDVLRIYTLELEDDGSPEYSKRYIRLAPTQKDVFVIRFKVTAGMLASNNGVLYTNYPISGCFDREKFHEVRFSSDFSKDTYCDVTVTTPGAYEYYVEYEPINQENSDNQPEPKRSSRSGYFVVEPRLYIDSPVKPSEPSAEKKEVQEHAPAKVLLPLDGLVIESAVGKWLGPLDTWDAHLQHMKAAGYNMIHFVPLQVRGASNSPYSIHDQTAFSDDLFNEQDVKKSSEEKAQLVNKQLHRIQDEYGIMCLSDVVWNHTSFDSVWLQDHPESGYNLDNSPHLQAAFELDTALINLVDDFSKYSLPTTLKSGQDLDAVMNVIKNNVFPDLKLWEYKVIDVVSEKKKLHEALLAAPKETAQSQVFENANLSGLASDQQAQLLAEHGIADRQSGSRYGKTVDIDVAVAFIKALGGVASGEQVSEDVAAKLVDIYGKVLDVYNLPFYKEYDEDVRIALENTKNRIEYTRLADHGPKWGEITKEKPIVDNYFTRLPKNDKTKGHSEGSLALANNGWIWNADPMQDFAGPQSAAYLRREVICWGDCVKLRYGSGADDNPYLWKHMKDYTEQTAKYFQGIRIDNCHSTPIHVAQYLLDAARKIRPDLYVVAELFTGSEDSDVTFVSKLGINSLIREAMQAWDAAELSRLVHRHGGKAIGSMDQALTTEQINYSLNGKEEEPALLVPVLQGSVPHALFMDCTHDNETPNEKRTPQDTLPNAALVAFSGSAIGSVKGYDDIYPRLLDIVNETRLYDLPKPDANVGINKVKPVLQKLHTEMIMDGYIECHVHHEGDYIIVHRQHPELHTGYLLVAHTAFPGRQNNRGSISPIKLRGTDVDIMFSATLDIESRENLSDEKTLRGLKSKIVSLSAPTIKSLSDNKGRYTEITVPASFPSGSIMVLKTWIQDNPAESYDLISSCSDDVFKNMTSLDMNVVLHRCEGEERDATGEGVYNIPGFGGLPYAGLEGFVSVLKPIIAHNDLGHPFCAHLREGTWAMDFIVDRLNRYIGNYPNLKRLKDWFASRFSEVKGLPDFLLPKYFALIIRTAYLKARAHAVSLMSPLVREGDRFIQSLALCSVQMYGIVPSSGLHPTEVTPSMAAGLPHFTHSYMRTWGRDVFIALRGLFLTTGNYDAAKRHIVSFASSLKHGLIPNLLDAVRHPRYNSRDSVWWFLQAVQDYYNMAPDGKSILQEKVSRRFPKNDTFVPPEEGFKYSCTVAEIIQEIFERHAAGIHFREHNAGEQLDRQMLDEGFNIDIDVDWESGTLIGGNRFNCGTWQDKMGESVKAGNKGLPATPRDGAPIEITGLLKSTLRWVVELHERGEFQWTSAKDKSGNERSYKDWNDLLQTNFERVYYVPLDEKEDSQYDVLSQIVNRRGIYKDVYKSKEPYTDYQFRSNVPVAMVVAPELFTPAKAMGSLELARSNLLGPLGMATLDPSDNEYHPDYHNSDDSDNKAVAKGWNYHQGPEWVWQTGYFLRAYLYFSLKTGEDQSKREKVMDIQRILLAHKREIESSPWAGLPELTNRNGNPCWDSCPTQACYDDEVVLIQSQNTSFNHRSPATSDQESAAINFFLFCANLISEEPMNNDQVIPLETHKLGLELRPHPQRGRGVFATCALKARTLVEISPILLVNHDEYSAHGQYTILDHYTYRWEGGYALALGLGSMFNHAKNPNVGFVRDIPNAVIRYFTLRAIEPAEELCISYGDHLWFEDTDAKSEASIDSSEDEWFGKIMVDDEEHHHHHH

SEQ ID NO: 350 - protein, 2104 aa, synthetic construct
MPVPITKARSGSTSEAAPGHPKAGAGTTRGQRPPPVQLPHERYADAPKTPGPKTPADEALDFFESTERGDARIQVYELRLEPDGGPNREAAYTRLPPAYKPYILRVTIDAGSPASRNGVFKTNFPLDGGKFSRDHFVERKLPTDFSKPIKIDLPISHAGAFVYWLEYDGSQGRIKAREGYFNIDPILTTYRRTPVIDHKTLAVQAQTGFVEPRESVNIPLDGIAMLTVVSKWMGPLPEWEQHFAEARTRGYNMLHYTPLQQRGESKSPYSIADQMAFDSGLFEDNYDGSKEEGTQRVKDILKLAKKQYGLMSLTDVVLNHTANNRFSPYNCPHLTPALELDNAIIDFSTDLASRGLPTLITSQKDIDTLIDALKEHIAKLNLWQYYVLNGQSEKEAVADAIAASKITPWTGPDIVGKSVAEIADIVRGEGNLEHVGEFAERFMITVNPSVAAGIVKTAFVDLGDATPAAYGEAWGRVVDVLNVDRYKEWEEDTKVAIENIINRVKWTRLDENGPKVGEINKTMPLVDTYFTRIPKNSRTAKHDPRALAVANNGWIWAADPLANFALRPSKAYLRREVIVWGDCVKLRYGDGPSDNPWLWKHMTEYVQSLAVLFDGFRIDNCHSTPLHVGVALLDAARAVNPDLYVCAELFTGSEEMDVHFVSRLGINSLIREAMNGWDPKDFARLLHRYGLGKPIGSMDESCLTSHEDLPPPTGKGATRPAAVTPIRGSAPHALMYDLTHDNESPLHKRSAEDALSTGALVTFGLSATGSNKGFDDLYPKLLDLVGDNRKYETSDGGKVDRGIAKVKKVLNQLHTEMVLAGFKEGHVHQEGDYIAIHRVHPATQKGYFLIAHTAFGSTKGSKARGDVNPISLKSTKARFILGASIDIPSYELEASKTTISGLPSTLKELAEVPPKVTDDGCELIVPEEFPPGSILLYETQLVGVDPNLESACAEGADEAFAELDLVDLNVVLHRCSGEERDATNGEIGTYNIGGLGDLVYCGLEGWMHPLRHVMNHNDLGHPLCENLRQGDWALGYVQSRLEKQTPTFPRLAKPAAWFKSRFDLIRAQVPGFLRPKYFAIVIFSAYKVARRIAIEQCSEFVVSGHDFTQNLALCQIQMHGLVQSASIDPGKVVPSLAAGLPHFAGGWARTWGRDVFISLRGLFLATGNYAAARAHILAFCATLKHGLIPNLLDALRTPRYNSRDSPWWMVQNIQDYCNMAPEGLALLEAPVKRRFPQDDTFVPWDDPRAYAYSSSVAEIIQEILQRHAKGISFREHNAGPNLDMQMSDEGFNIEVKVDWETGFTLGGNWKNCGTWMDKMGESQKAGTKGTPGTPRDGAPVEITGLLFSTLRWLDGLGSKFPFKGVQATIDGQEKLVTYREWATLIQNAFERYYYVPLNPVEDPDYVVDPRIINQRGIYKDVYGSGAGREWSDYQLRCNFPIAMVVAPELFNPEHALGALKIADKRLRGPLGMKTLDEGDSQYRGYYDNSNDSDDASIAKGLNYHQGPEWGWPLGYFLRAYLKFDQIAGAGKNDQAEMDAPTAAEDQTPSTPELRRTYLSLLSHNAIVELVLSLNADVTSSLFPSDLAGEVRRMQSSSATDDVDGEGSPDPQPAPPPRAPSVPPAGGPRTRAGAARAYAAALASKDTPSTRPSPAPGGVSFPQTPEPQQPQQSPPPFAPISQPYPSLIVHLPGGSPPIPSPTTPTNHASTAANPLPGIGPVRNTPAKKTRTGVIGGYNPITSIGPSSEGLPSYEEMIVLALMEGQDDEGVAPKDVFAWMSARWPLNANFRPSASQALQKAYKRGRLEKVGTKYKLNPNWHGGATTNRTTRRPQSMAEVPGNYAWAPPPVPPVVSVPPDPRTRHSITPNPSTEPPASQQTQTEFDAQAQVASLLKALQEAGPGPSNPNPNSSGPEQNQPPNVEEGVHTHLPHMSIPGAADGARPIQSSPETAVIVPTTGSPSALGPSQSPVQISQLPASMPPLPAPVQPGLPTPPIQAIMSPVQSQNGILPLQNPTSPPIQNGVSSQPTTNTSLSQPQPSQPPQPQVLHQPHVPPQNSAIPQPPHPMPPGVPMTYPSFHTTPSALQASLVTLASQLANMSKTNQGA

SEQ ID NO: 351 - protein, 2110 aa, synthetic construct
MPVPITKARSGSTSEAAPGHPKAGAGTTRGQRPPPVQLPHERYADAPKTPGPKTPADEALDFFESTERGDARIQVYELRLEPDGGPNREAAYTRLPPAYKPYILRVTIDAGSPASRNGVFKTNFPLDGGKFSRDHFVERKLPTDFSKPIKIDLPISHAGAFVYWLEYDGSQGRIKAREGYFNIDPILTTYRRTPVIDHKTLAVQAQTGFVEPRESVNIPLDGIAMLTVVSKWMGPLPEWEQHFAEARTRGYNMLHYTPLQQRGESKSPYSIADQMAFDSGLFEDNYDGSKEEGTQRVKDILKLAKKQYGLMSLTDVVLNHTANNRFSPYNCPHLTPALELDNAIIDFSTDLASRGLPTLITSQKDIDTLIDALKEHIAKLNLWQYYVLNGQSEKEAVADAIAASKITPWTGPDIVGKSVAEIADIVRGEGNLEHVGEFAERFMITVNPSVAAGIVKTAFVDLGDATPAAYGEAWGRVVDVLNVDRYKEWEEDTKVAIENIINRVKWTRLDENGPKVGEINKTMPLVDTYFTRIPKNSRTAKHDPRALAVANNGWIWAADPLANFALRPSKAYLRREVIVWGDCVKLRYGDGPSDNPWLWKHMTEYVQSLAVLFDGFRIDNCHSTPLHVGVALLDAARAVNPDLYVCAELFTGSEEMDVHFVSRLGINSLIREAMNGWDPKDFARLLHRYGLGKPIGSMDESCLTSHEDLPPPTGKGATRPAAVTPIRGSAPHALMYDLTHDNESPLHKRSAEDALSTGALVTFGLSATGSNKGFDDLYPKLLDLVGDNRKYETSDGGKVDRGIAKVKKVLNQLHTEMVLAGFKEGHVHQEGDYIAIHRVHPATQKGYFLIAHTAFGSTKGSKARGDVNPISLKSTKARFILGASIDIPSYELEASKTTISGLPSTLKELAEVPPKVTDDGCELIVPEEFPPGSILLYETQLVGVDPNLESACAEGADEAFAELDLVDLNVVLHRCSGEERDATNGEIGTYNIGGLGDLVYCGLEGWMHPLRHVMNHNDLGHPLCENLRQGDWALGYVQSRLEKQTPTFPRLAKPAAWFKSRFDLIRAQVPGFLRPKYFAIVIFSAYKVARRIAIEQCSEFVVSGHDFTQNLALCQIQMHGLVQSASIDPGKVVPSLAAGLPHFAGGWARTWGRDVFISLRGLFLATGNYAAARAHILAFCATLKHGLIPNLLDALRTPRYNSRDSPWWMVQNIQDYCNMAPEGLALLEAPVKRRFPQDDTFVPWDDPRAYAYSSSVAEIIQEILQRHAKGISFREHNAGPNLDMQMSDEGFNIEVKVDWETGFTLGGNWKNCGTWMDKMGESQKAGTKGTPGTPRDGAPVEITGLLFSTLRWLDGLGSKFPFKGVQATIDGQEKLVTYREWATLIQNAFERYYYVPLNPVEDPDYVVDPRIINQRGIYKDVYGSGAGREWSDYQLRCNFPIAMVVAPELFNPEHALGALKIADKRLRGPLGMKTLDEGDSQYRGYYDNSNDSDDASIAKGLNYHQGPEWGWPLGYFLRAYLKFDQIAGAGKNDQAEMDAPTAAEDQTPSTPELRRTYLSLLSHNAIVELVLSLNADVTSSLFPSDLAGEVRRMQSSSATDDVDGEGSPDPQPAPPPRAPSVPPAGGPRTRAGAARAYAAALASKDTPSTRPSPAPGGVSFPQTPEPQQPQQSPPPFAPISQPYPSLIVHLPGGSPPIPSPTTPTNHASTAANPLPGIGPVRNTPAKKTRTGVIGGYNPITSIGPSSEGLPSYEEMIVLALMEGQDDEGVAPKDVFAWMSARWPLNANFRPSASQALQKAYKRGRLEKVGTKYKLNPNWHGGATTNRTTRRPQSMAEVPGNYAWAPPPVPPVVSVPPDPRTRHSITPNPSTEPPASQQTQTEFDAQAQVASLLKALQEAGPGPSNPNPNSSGPEQNQPPNVEEGVHTHLPHMSIPGAADGARPIQSSPETAVIVPTTGSPSALGPSQSPVQISQLPASMPPLPAPVQPGLPTPPIQAIMSPVQSQNGILPLQNPTSPPIQNGVSSQPTTNTSLSQPQPSQPPQPQVLHQPHVPPQNSAIPQPPHPMPPGVPMTYPSFHTTPSALQASLVTLASQLANMSKTNQGAHHHHHH

SEQ ID NO: 352 - protein, 1363 aa, synthetic construct
MAKTSDETSFASSMPNFSRNIEIDITIPHAGAYAYYITYAGLPPLAEVLESIDTAEPEVATHKTDLFYIDVAPYLTINSSSLPLDGLSVFTVLSKFMGKYPKDWSSHFHGISERGYNMVHFTPLQVRGSSNSPYSLFDQLAWDSQFFPNGESDIAKLVDDLEKNYGLLSLTDVVLNHTAHNSKWLEEHPEAGYNLTTAPWLESAYILDTKLLQLSSKLTEKALPTVLNDEKDLLKIMDAIKTDVIFDMRLWEFYTLDIEANATAAVAAWKSGKYSFPEEKDIEMINKPLKEQAKYLTDNGLSGNDRMGSRFRRHIDPEISGGLLALHFGRFESDSTNGADEGAVKTKISTILDAANLPFYKEYDAEVADILQQLFNRIKYMRLEENGPKLGEITAENPLIETYFTRLPKNETTAQHSEDDLALANNGWVWGGNALIDNAGPNSRVYLRREVIVWGDCVKLRYGSSPEDSPYLWDRMTKYVRLLTKYFAGLRIDNCHSTPIHVAEHILDEARRIRPNVYVVAELFSGSEETDYVFVKRLGLSSLIREAMQAWSTGELNGTKSTESTSEHTRNNSITKSRELIRNIKPVPVQALFMDCTHDNETPFQKRDARDTLPNAALVSMCASASGSVMGYDEIYPKIIDLVGETRLYVSEASSSPSVKIGAGKNGIAGVKKLLNQIHTLMGKDGYDETHVHHEDQYVTVHRVHPKSRKGYFLIAHTAFPGYGNGNGAFNPVHLTGTKAKHLGSWMLEVDTSEEALAQVQNDKTYLNGLPSQVVDLSSVKLDEAAGAFNELDLVDLNFVLYRCEAEERSTSNGQDERFDALRDIPSFLIPRYFALVIRTAYRASWDRAMNLMSPGVQEGQWFMQSLAMVSVQQTGIVPSASLYPKTQVPSLAAGLPHFAVEWARCWGRDVMISLRGLYLGTGRFDEAKEHIKAFGSVMKHGMIPNLLGSGGNPRYNARDSIWFFLQCIQEYTKIVPDGISILNEKVKRRFLPYDDTWFAVDDSRAYSNESTIAEIIQEALQRHAAGISFREANAGSAIDSQMSDEGFNIDIKVDWENGFIFGGNQHNCGTWMDKMGESVKARSKGVPGTPRDGAAVEITGMLYSTLKWVSELNATGKFAKPGVEKADGSAISFADWAALVKTNFERCYFVPKDSVDDSKYDVNPSVINRRGIYKDLYRSGKEYEDYQLRPNFPIAMCVAPDLFEPAHALSALFVAESVLRGPTGMATLDPTDLNYRPYYINSEDSTDFATAKGRNYHQGPEWLWPTGFFLRALLKFDLMRRGDGAAARTESFQQITQRLIGCKKMIRESPWAGLAELTQKNGEFCGDSCPTQAWSASCLIDLYMDAAMEHHSHDMSIPIHRK

SEQ ID NO: 353 - protein, 1369 aa, synthetic construct
MAKTSDETSFASSMPNFSRNIEIDITIPHAGAYAYYITYAGLPPLAEVLESIDTAEPEVATHKTDLFYIDVAPYLTINSSSLPLDGLSVFTVLSKFMGKYPKDWSSHFHGISERGYNMVHFTPLQVRGSSNSPYSLFDQLAWDSQFFPNGESDIAKLVDDLEKNYGLLSLTDVVLNHTAHNSKWLEEHPEAGYNLTTAPWLESAYILDTKLLQLSSKLTEKALPTVLNDEKDLLKIMDAIKTDVIFDMRLWEFYTLDIEANATAAVAAWKSGKYSFPEEKDIEMINKPLKEQAKYLTDNGLSGNDRMGSRFRRHIDPEISGGLLALHFGRFESDSTNGADEGAVKTKISTILDAANLPFYKEYDAEVADILQQLFNRIKYMRLEENGPKLGEITAENPLIETYFTRLPKNETTAQHSEDDLALANNGWVWGGNALIDNAGPNSRVYLRREVIVWGDCVKLRYGSSPEDSPYLWDRMTKYVRLLTKYFAGLRIDNCHSTPIHVAEHILDEARRIRPNVYVVAELFSGSEETDYVFVKRLGLSSLIREAMQAWSTGELNGTKSTESTSEHTRNNSITKSRELIRNIKPVPVQALFMDCTHDNETPFQKRDARDTLPNAALVSMCASASGSVMGYDEIYPKIIDLVGETRLYVSEASSSPSVKIGAGKNGIAGVKKLLNQIHTLMGKDGYDETHVHHEDQYVTVHRVHPKSRKGYFLIAHTAFPGYGNGNGAFNPVHLTGTKAKHLGSWMLEVDTSEEALAQVQNDKTYLNGLPSQVVDLSSVKLDEAAGAFNELDLVDLNFVLYRCEAEERSTSNGQDERFDALRDIPSFLIPRYFALVIRTAYRASWDRAMNLMSPGVQEGQWFMQSLAMVSVQQTGIVPSASLYPKTQVPSLAAGLPHFAVEWARCWGRDVMISLRGLYLGTGRFDEAKEHIKAFGSVMKHGMIPNLLGSGGNPRYNARDSIWFFLQCIQEYTKIVPDGISILNEKVKRRFLPYDDTWFAVDDSRAYSNESTIAEIIQEALQRHAAGISFREANAGSAIDSQMSDEGFNIDIKVDWENGFIFGGNQHNCGTWMDKMGESVKARSKGVPGTPRDGAAVEITGMLYSTLKWVSELNATGKFAKPGVEKADGSAISFADWAALVKTNFERCYFVPKDSVDDSKYDVNPSVINRRGIYKDLYRSGKEYEDYQLRPNFPIAMCVAPDLFEPAHALSALFVAESVLRGPTGMATLDPTDLNYRPYYINSEDSTDFATAKGRNYHQGPEWLWPTGFFLRALLKFDLMRRGDGAAARTESFQQITQRLIGCKKMIRESPWAGLAELTQKNGEFCGDSCPTQAWSASCLIDLYMDAAMEHHSHDMSIPIHRKHHHHHH

SEQ ID NO: 354 - protein, 822 aa, synthetic construct
MRIRHSFLCVFMLAAGSHARATEFNASFLSIDGRNDVDLSQFAQADYTLPGTYLLDVQVNDVFFGLQSIEFVAHDDGQGARACVAPELVAQFGLKKSLVENLPRTMGGRCADLASLDGVTIRYQKGEGRLKITIAQAALEFADASYLPPERWSDGVDGAMLDYRLLANANHAFGRGAQQNNSVQAYGTIGANRGAWRFRGDYQAQTRAGGAVYAERAFRFNQLYAYRALPSIRSTLSFGEIYVDSDIFSTFSMSGVAMKSDDRMLPPSMRGYAPLVTGVARTNAIVKVMQDSRVLYMTKVSPGAFALSNLNTSVQGTLDVVVEEEDGTVQRFQVATASVPFLAREGQLRYKTAIGQPRTFGGAGITPWFGFAEAAYGLPFDVTVYGGLIAASGYTSVAFGVGRDFGRFGALSADVTHARATLWWNGRTKRGNSYRINYSKHVDALDADVRFFGYRFSERDYTNFQQFSGDPTASGLANGKQRYSAMLSKRFGDTSTYFSYDQTTYWARPSDRRIGVTLTRAFSLGALKSVNLGFSAFRTQGAGGGGNQVSLTATLPLGERQTLTSSVSAGEGGTSVNAGYLYDGANGRTYQLYGGTTDGRASANASLRQRTPSYQLTAQASTVANAYASASLEVDGSFVATRYGVTAHANGNAGDTRLLVSTDGVPGVPLSGSYARTNARGYAVIDGVSPYNVYDATVSVEKLGLDTDVTNPIQRTVLTDGAIGYIRFNAARGRNVFVTLTGDGGAPVPFGASVQDAATGKELGIVGEAGAAYLTQVQPRAKLVVRAGAKTICTPAALPDTLQLDGTPAPVACEAAAQHASR

SEQ ID NO: 355 - protein, 807 aa, synthetic construct
TEFNASFLSIDGRNDVDLSQFAQADYTLPGTYLLDVQVNDVFFGLQSIEFVAHDDGQGARACVAPELVAQFGLKKSLVENLPRTMGGRCADLASLDGVTIRYQKGEGRLKITIAQAALEFADASYLPPERWSDGVDGAMLDYRLLANANHAFGRGAQQNNSVQAYGTIGANRGAWRFRGDYQAQTRAGGAVYAERAFRFNQLYAYRALPSIRSTLSFGEIYVDSDIFSTFSMSGVAMKSDDRMLPPSMRGYAPLVTGVARTNAIVKVMQDSRVLYMTKVSPGAFALSNLNTSVQGTLDVVVEEEDGTVQRFQVATASVPFLAREGQLRYKTAIGQPRTFGGAGITPWFGFAEAAYGLPFDVTVYGGLIAASGYTSVAFGVGRDFGRFGALSADVTHARATLWWNGRTKRGNSYRINYSKHVDALDADVRFFGYRFSERDYTNFQQFSGDPTASGLANGKQRYSAMLSKRFGDTSTYFSYDQTTYWARPSDRRIGVTLTRAFSLGALKSVNLGFSAFRTQGAGGGGNQVSLTATLPLGERQTLTSSVSAGEGGTSVNAGYLYDGANGRTYQLYGGTTDGRASANASLRQRTPSYQLTAQASTVANAYASASLEVDGSFVATRYGVTAHANGNAGDTRLLVSTDGVPGVPLSGSYARTNARGYAVIDGVSPYNVYDATVSVEKLGLDTDVTNPIQRTVLTDGAIGYIRFNAARGRNVFVTLTGDGGAPVPFGASVQDAATGKELGIVGEAGAAYLTQVQPRAKLVVRAGAKTICTPAALPDTLQLDGTPAPVACEAAAQHASRHHHHHH

SEQ ID NO: 356 - protein, 649 aa, synthetic construct
MSYLTFKKKELINLEYSLDREFLGTNRGGGYSSSTLVFCNTRKYHGLLVVPLENFRGQNHVLLSSLDETIIQHGRDFNFAVHQYPETYEPRGHKYIVDVSYEKAFTVIYQVGGVTLKKEILMVHNEPQVLVRYTLLEAHSPTKLRLNPLLAFRDIHSLSKVNDLANKNIEKIEGGIKTKLYPDFPYLHLQLSQDTDFEYQPNWNCNIFYKKEKERGYDYTEDLLTLGYFELNIKKGESVVFSASTEENNSKQLKQKFTRLINQRRERNSFEECLNYSATQFLIQKAGEIRVKEGYHWYNSNTRDTFIALPGIALAENTQKTFDEVLTSVKKYFSNGLFEKTVSTHVAERQYDADTSLWFFWTIQQYEKYTKKTPKQLWKDFGELMRTILETYKNGQHKSIRMDNNGLIWSEDISRPLTWVDAQFDGGAIVPRNGYVVEVNALWYNAVCYALALAEASKDKDFLKEWSDLPMMIAENFNQTFWISDKKYLADYVNYAHQNRDVRPNQLIPCALAYSPLDERSKKRVLDIVRQELLTPRGLRSLSPKNKSYESVSIGNVYDRDRATFNGSVHPWFIGFYIEAHLKIYGKTYIPDAEAILADFEEELSGHGVSTISAIYDGNPPFRSRGCISKARNVAEILRAKQLTMNYES

SEQ ID NO: 357 - protein, 655 aa, synthetic construct
MSYLTFKKKELINLEYSLDREFLGTNRGGGYSSSTLVFCNTRKYHGLLVVPLENFRGQNHVLLSSLDETIIQHGRDFNFAVHQYPETYEPRGHKYIVDVSYEKAFTVIYQVGGVTLKKEILMVHNEPQVLVRYTLLEAHSPTKLRLNPLLAFRDIHSLSKVNDLANKNIEKIEGGIKTKLYPDFPYLHLQLSQDTDFEYQPNWNCNIFYKKEKERGYDYTEDLLTLGYFELNIKKGESVVFSASTEENNSKQLKQKFTRLINQRRERNSFEECLNYSATQFLIQKAGEIRVKEGYHWYNSNTRDTFIALPGIALAENTQKTFDEVLTSVKKYFSNGLFEKTVSTHVAERQYDADTSLWFFWTIQQYEKYTKKTPKQLWKDFGELMRTILETYKNGQHKSIRMDNNGLIWSEDISRPLTWVDAQFDGGAIVPRNGYVVEVNALWYNAVCYALALAEASKDKDFLKEWSDLPMMIAENFNQTFWISDKKYLADYVNYAHQNRDVRPNQLIPCALAYSPLDERSKKRVLDIVRQELLTPRGLRSLSPKNKSYESVSIGNVYDRDRATFNGSVHPWFIGFYIEAHLKIYGKTYIPDAEAILADFEEELSGHGVSTISAIYDGNPPFRSRGCISKARNVAEILRAKQLTMNYESHHHHHH

SEQ ID NO: 358 - protein, 716 aa, synthetic construct
MAFSIARAVVIKHGNVFFLAPEEGEIPFTSGHGFGLYYHDCRYLNGYELLVGDVHPEPLGARASEGARAMFRLTTPDFRMADGRAVPRETVGLKWERVIDSEQCLLQERLEIQNFGHEPVEFPLAMRFRAGFEDVFEIRGLLREVKGKLYRPGWRDKCLCFSYEGGDHVTRSLAVHFDPLPPDVKGSAVQFHLRLQPNEINVIRIVLAIAESDRVPATVCAQVNIRRDLPTVVRVMRSECEEWMMRETEVHTDSEAVNRIMKRSLMDLVMLRTTIGGDEFFAAGVPWYATLFGRDSLVTSLQTLAYGAETAADSLRLLARYQGDRVSEWRDEEPGKILHELRVGELARMNKIPHTPYYGTIDATPLFIILMSRYADWTGDLSLFTELRPHVERALEWIDRYGDAAGDGYVSYLGHSDKGLLHQGWKDSGDAIVTADGRFAEPPIALVEVQGYVYLAKRSIADLYERVGEPDRAALLREEARELRRRFNRDFWMEDEGCYALALETGGRPCRVLSSNAGQALWTGIVDEEKAGRVVDRLMRPDLFSGWGVRTLSYKERRYNPMGYHLGTVWPHDNSLIAAGFRRYGFDEAAGRIFAGLVDAAREFEDFRLPELFTGFGREEYGVPVRYPVACHPQAWAAGSVPFMIETLLGLEPEALEHRLRVVRPFLPDFIGRLDLRHLQVGKGAADLRFSRKADGRVDVEVLNIAGTLTVEVEKQ

SEQ ID NO: 359 - protein, 722 aa, synthetic construct
MAFSIARAVVIKHGNVFFLAPEEGEIPFTSGHGFGLYYHDCRYLNGYELLVGDVHPEPLGARASEGARAMFRLTTPDFRMADGRAVPRETVGLKWERVIDSEQCLLQERLEIQNFGHEPVEFPLAMRFRAGFEDVFEIRGLLREVKGKLYRPGWRDKCLCFSYEGGDHVTRSLAVHFDPLPPDVKGSAVQFHLRLQPNEINVIRIVLAIAESDRVPATVCAQVNIRRDLPTVVRVMRSECEEWMMRETEVHTDSEAVNRIMKRSLMDLVMLRTTIGGDEFFAAGVPWYATLFGRDSLVTSLQTLAYGAETAADSLRLLARYQGDRVSEWRDEEPGKILHELRVGELARMNKIPHTPYYGTIDATPLFIILMSRYADWTGDLSLFTELRPHVERALEWIDRYGDAAGDGYVSYLGHSDKGLLHQGWKDSGDAIVTADGRFAEPPIALVEVQGYVYLAKRSIADLYERVGEPDRAALLREEARELRRRFNRDFWMEDEGCYALALETGGRPCRVLSSNAGQALWTGIVDEEKAGRVVDRLMRPDLFSGWGVRTLSYKERRYNPMGYHLGTVWPHDNSLIAAGFRRYGFDEAAGRIFAGLVDAAREFEDFRLPELFTGFGREEYGVPVRYPVACHPQAWAAGSVPFMIETLLGLEPEALEHRLRVVRPFLPDFIGRLDLRHLQVGKGAADLRFSRKADGRVDVEVLNIAGTLTVEVEKQHHHHHH

SEQ ID NO: 360 - protein, 718 aa, synthetic construct
MSEGNRCEKIQVDGEWYILCSSVPLTTRKLTLKGDRAFVITDAYGDVPVAYPSELGYYWRDTRFLSGFEMYLNGARPVLLSSDIDADSKCIIVEMTNTDFRVGNKIVHRTSLYLSKTITFEGDDLVQVVVLRNLSLFDLPCDLQFIMEADFKDIFEVRGSERDKRGESLEPIIGKGYVEYRYKGLDGLLRSCNVQFSPSDFCFEGNAARFKFVLEPKKEIEIVIRASCKIEDSLIFCTKNTPKPGFESHVDFSSSNQLLSSVVGRSLKDLEMMLSDFDGHAIPMAGIPWYCALFGRDSIITALELLPWYPELARNVLAVLSRYQARDFDDFTDSEPGKILHELREGEMARLREIPFVPYYGTADATLLYITLAAEYVKTTGDLDFLKSIWENINLATKWMEHYGDINGDGFVEYMSRSPVGLRNQGWKDSHDSIHHVDGALAEPPISVVEVQGYKYQALKALSFLSKCMGNVGEAERFDRRAQELASKLEGVFWMPSKGFYALALDKNGKRCEIISSNAGHLLFACAVSRERAAKVAERLLSDEMFTGYGIRTLSRHEVRYNPMSYHNGSVWPHDCAIICRGLVNYGFKREALKIFRGILEAVALFPDYRVPELFCGFPRDSEKPPVPYPVACSPQAWSSASVIFMLTSLLGIEVDAFNKILTFNNPVLPSEIQELEIKDWFTPFFGEFSFKFVNVGERVAVETLKKPHDWKVLVVVG

SEQ ID NO: 361 - protein, 724 aa, synthetic construct
MSEGNRCEKIQVDGEWYILCSSVPLTTRKLTLKGDRAFVITDAYGDVPVAYPSELGYYWRDTRFLSGFEMYLNGARPVLLSSDIDADSKCIIVEMTNTDFRVGNKIVHRTSLYLSKTITFEGDDLVQVVVLRNLSLFDLPCDLQFIMEADFKDIFEVRGSERDKRGESLEPIIGKGYVEYRYKGLDGLLRSCNVQFSPSDFCFEGNAARFKFVLEPKKEIEIVIRASCKIEDSLIFCTKNTPKPGFESHVDFSSSNQLLSSVVGRSLKDLEMMLSDFDGHAIPMAGIPWYCALFGRDSIITALELLPWYPELARNVLAVLSRYQARDFDDFTDSEPGKILHELREGEMARLREIPFVPYYGTADATLLYITLAAEYVKTTGDLDFLKSIWENINLATKWMEHYGDINGDGFVEYMSRSPVGLRNQGWKDSHDSIHHVDGALAEPPISVVEVQGYKYQALKALSFLSKCMGNVGEAERFDRRAQELASKLEGVFWMPSKGFYALALDKNGKRCEIISSNAGHLLFACAVSRERAAKVAERLLSDEMFTGYGIRTLSRHEVRYNPMSYHNGSVWPHDCAIICRGLVNYGFKREALKIFRGILEAVALFPDYRVPELFCGFPRDSEKPPVPYPVACSPQAWSSASVIFMLTSLLGIEVDAFNKILTFNNPVLPSEIQELEIKDWFTPFFGEFSFKFVNVGERVAVETLKKPHDWKVLVVVGHHHHHH

SEQ ID NO: 362 - protein, 619 aa, synthetic construct
MIVNAPAQAIAAPDGSMGDAPVQGVYQSDVRALRLRALSVTGSALEHLSVERCGAAASRHRALARGLDRGGADPRSIITTDRTLVPADDGTVLTETLTVRIAQDRPVSTTLTLELAADLSPLAMIKGGERLSDLPWTVAGGDTASVRRGQVRAVVNAPEARLETVGTRLRLHWDVSVAADIPFTASWSLTASDDDAVVQPAPALPARKRRGTPDARAAAWLEVALDDLDHLLLARRATPTQPFAAAGAPWFLTLFGRDSLWTSRLLLEAADPWSLALAEGTLRTLADLQGRRYDVDTAEQPGKIMHELRREANAVDADTVLPPLYYGTIDATELWILTLTDARRAGLAEPVVRELLDPLRRALSWLTTCADTDGDGFIEYVDGTGRGLANQGWKDSGDSIQFADGRIAQGPIALVEVQAYAHAAALAGAELLRDFGNGDDAVLAATATTWAAALRERFAEAFWRGDTADGYPVIALDARKQQVDSLASNIGHLLGTGILDRAQSRRVAELLVSPELNSGYGVRTLASTMAGYWPLSYHGGSVWTHDTAIAIDGLLRDGFTSQARTLAEALLTAAPCFGFRMPELFSGTPADDGAPVPYPASCRPQAWAAAAGAVVARAL

SEQ ID NO: 363 - protein, 625 aa, synthetic construct
MIVNAPAQAIAAPDGSMGDAPVQGVYQSDVRALRLRALSVTGSALEHLSVERCGAAASRHRALARGLDRGGADPRSIITTDRTLVPADDGTVLTETLTVRIAQDRPVSTTLTLELAADLSPLAMIKGGERLSDLPWTVAGGDTASVRRGQVRAVVNAPEARLETVGTRLRLHWDVSVAADIPFTASWSLTASDDDAVVQPAPALPARKRRGTPDARAAAWLEVALDDLDHLLLARRATPTQPFAAAGAPWFLTLFGRDSLWTSRLLLEAADPWSLALAEGTLRTLADLQGRRYDVDTAEQPGKIMHELRREANAVDADTVLPPLYYGTIDATELWILTLTDARRAGLAEPVVRELLDPLRRALSWLTTCADTDGDGFIEYVDGTGRGLANQGWKDSGDSIQFADGRIAQGPIALVEVQAYAHAAALAGAELLRDFGNGDDAVLAATATTWAAALRERFAEAFWRGDTADGYPVIALDARKQQVDSLASNIGHLLGTGILDRAQSRRVAELLVSPELNSGYGVRTLASTMAGYWPLSYHGGSVWTHDTAIAIDGLLRDGFTSQARTLAEALLTAAPCFGFRMPELFSGTPADDGAPVPYPASCRPQAWAAAAGAVVARALHHHHHH

SEQ ID NO: 364 - protein, 730 aa, synthetic construct
MILEAAEEDVEEIISVNDQFYILASSSMADDRTRVLKHGETFGVYDRYGDIQPVGRGTQGVFHQGTRFLSRQELFLNNDRPMLLSSTVKEDNALLAVDLTNPDLYRDGRIAIPRGSVHVFRSRFLWNGVSYERFRLSNYSLAPVKMTLSIRFEADFADIFEVRGKKRERKGRMLPNVLRKDLLVLSYEGLDEVTREARIQFSPEPLEITASQATFDIELDPKGETTVAVMVACDVGDSHRPLLAYDAAMAEAGLAFGAEQTEDCTIQTSNAQFNEWWNRSVLDVRMMVTDTNEGPYPYAGVPWFSTPFGRDGVITALECLWIRPELARGVLAYLASTQAKEVNPGQDAEPGKILHETRKGEMAALNEIPFGLYYGSVDSTPLFVMLAGAYYERTADLAFIQSIWANLEAALTWMDTFGDPDRDGFVEYVRKSPTGLDNQGWKDSHDSISHEDGSLAEGPIALCEVQGYVYDAKVQASKLADALGYIDRASQLRRQARSLKERFEEAFWCEESSTYALALDGQKRPCQVKTSNAGHCLYTGIASDEHARRVAETLMSDELFSGWGVRTLADSERRYNPMSYHNGSIWPHDNAMIAVGLARYGLKSGVEKIMTGMFEVSLVLDFHRLPELFCGFVRRPGQGLTRYPVACNPQAWAAGSAFMALQACLGLSIIASEQKVVFNYPILPEFIDKMQIKNLKVGTASVDLLLRRHDLDVGITVIRRAGKVEVVSVK

SEQ ID NO: 365 - protein, 736 aa, synthetic construct
MILEAAEEDVEEIISVNDQFYILASSSMADDRTRVLKHGETFGVYDRYGDIQPVGRGTQGVFHQGTRFLSRQELFLNNDRPMLLSSTVKEDNALLAVDLTNPDLYRDGRIAIPRGSVHVFRSRFLWNGVSYERFRLSNYSLAPVKMTLSIRFEADFADIFEVRGKKRERKGRMLPNVLRKDLLVLSYEGLDEVTREARIQFSPEPLEITASQATFDIELDPKGETTVAVMVACDVGDSHRPLLAYDAAMAEAGLAFGAEQTEDCTIQTSNAQFNEWWNRSVLDVRMMVTDTNEGPYPYAGVPWFSTPFGRDGVITALECLWIRPELARGVLAYLASTQAKEVNPGQDAEPGKILHETRKGEMAALNEIPFGLYYGSVDSTPLFVMLAGAYYERTADLAFIQSIWANLEAALTWMDTFGDPDRDGFVEYVRKSPTGLDNQGWKDSHDSISHEDGSLAEGPIALCEVQGYVYDAKVQASKLADALGYIDRASQLRRQARSLKERFEEAFWCEESSTYALALDGQKRPCQVKTSNAGHCLYTGIASDEHARRVAETLMSDELFSGWGVRTLADSERRYNPMSYHNGSIWPHDNAMIAVGLARYGLKSGVEKIMTGMFEVSLVLDFHRLPELFCGFVRRPGQGLTRYPVACNPQAWAAGSAFMALQACLGLSIIASEQKVVFNYPILPEFIDKMQIKNLKVGTASVDLLLRRHDLDVGITVIRRAGKVEVVSVKHHHHHH

SEQ ID NO: 366 - protein, 1073 aa, synthetic construct
MQVKQVLRETGVVLAAGLLYGSAAFAQSSVIIGTVINTEDKKPAADVVVTATSPNLQGEQTVVTDAQGNYRIPQLPPGTYTLRFEKETFKPYARPEIQLLLNRTIRVNVELLPDSFTQTVDVIGTPPTIDVGSTNQGVNVDQEFIKRIAVARPVGKGGATRSFESLAELAPGAQADQYGVSINGTTSPENGYVVDGLSTNDPAFGVNASPLSIEFVQDVNIITGGYMPEFGRSTGGVINAVTRSGSNEFHGSVFANWTPGALEGKRKLVVEDGSTITGLNALSNLGDFGATLGGPILKDKLWFFAGFAPSFQRYQHTRALNAFQLTAPFDPTPGATNTVAKDANGSSVVQEIPGSERKYFADARTIQFMGKLTYLINQDHNVSFALNGTPTVSGGLGKLRINPKTGGLPGAQISRPEDIGQVEDRANTTALALKYAGAFMDKKVLVDANVGWFHQTAGVTPADGTALGSTDGLAGYSLAQYTRRRTLTTFESVPNAAEYCGATTAEQLLRCPSTNYLVGGPGFMSEATLDRYQINAKATYLLNALGTHVFKAGVDTEFLNFDQKKAYSGSVYLVEAGSASDQMYQGVSRHIWSDNRRYGYQTAPDTPVFETLQQSSTSGTTIGGFLQDSWSIANRVTLNLGVRYDVQNMYGGDGNLALKLGNQWSPRLGAVVDPFANGRAKLFVNFARYYEQVPLNMMDRAFPGERRFSARRYLTEPGGAEAGCDPSTLEGQRGGCSDRQYIVPRAESTLNPNQYFSGGLVQSEPVDPDIKPQSSDELVVGGEYELLANTRVGASYTHRDMNSVIEDMSRDDGNTYFLGNPGSGFAKEFPKPVRDYDAVTVYLNRTFTDGWLAQASYTWSRLYGNYPGLFRPENNQLDPNILADFDLIALLDNRTGLLPFDRTHAIKVFGAKEFNISNALSASIGLSYRGNSGTPISYLGAYPGYGQDETFILPRGSGGRTPWVNSVDSNVGVNYRVSKDSVVSFTLDVFNLFNFQTADRVDQSYTFSEVLPVYDPKTGKAGTAADLPTKTSPGSVPSAEGGVLAYDDVNQNFKNPDRYQAPRQIRFGVRYTF

SEQ ID NO: 367 - protein, 1053 aa, synthetic construct
QSSVIIGTVINTEDKKPAADVVVTATSPNLQGEQTVVTDAQGNYRIPQLPPGTYTLRFEKETFKPYARPEIQLLLNRTIRVNVELLPDSFTQTVDVIGTPPTIDVGSTNQGVNVDQEFIKRIAVARPVGKGGATRSFESLAELAPGAQADQYGVSINGTTSPENGYVVDGLSTNDPAFGVNASPLSIEFVQDVNIITGGYMPEFGRSTGGVINAVTRSGSNEFHGSVFANWTPGALEGKRKLVVEDGSTITGLNALSNLGDFGATLGGPILKDKLWFFAGFAPSFQRYQHTRALNAFQLTAPFDPTPGATNTVAKDANGSSVVQEIPGSERKYFADARTIQFMGKLTYLINQDHNVSFALNGTPTVSGGLGKLRINPKTGGLPGAQISRPEDIGQVEDRANTTALALKYAGAFMDKKVLVDANVGWFHQTAGVTPADGTALGSTDGLAGYSLAQYTRRRTLTTFESVPNAAEYCGATTAEQLLRCPSTNYLVGGPGFMSEATLDRYQINAKATYLLNALGTHVFKAGVDTEFLNFDQKKAYSGSVYLVEAGSASDQMYQGVSRHIWSDNRRYGYQTAPDTPVFETLQQSSTSGTTIGGFLQDSWSIANRVTLNLGVRYDVQNMYGGDGNLALKLGNQWSPRLGAVVDPFANGRAKLFVNFARYYEQVPLNMMDRAFPGERRFSARRYLTEPGGAEAGCDPSTLEGQRGGCSDRQYIVPRAESTLNPNQYFSGGLVQSEPVDPDIKPQSSDELVVGGEYELLANTRVGASYTHRDMNSVIEDMSRDDGNTYFLGNPGSGFAKEFPKPVRDYDAVTVYLNRTFTDGWLAQASYTWSRLYGNYPGLFRPENNQLDPNILADFDLIALLDNRTGLLPFDRTHAIKVFGAKEFNISNALSASIGLSYRGNSGTPISYLGAYPGYGQDETFILPRGSGGRTPWVNSVDSNVGVNYRVSKDSVVSFTLDVFNLFNFQTADRVDQSYTFSEVLPVYDPKTGKAGTAADLPTKTSPGSVPSAEGGVLAYDDVNQNFKNPDRYQAPRQIRFGVRYTFHHHHHH

SEQ ID NO: 368 - protein, 907 aa, synthetic construct
MSINFGREICGDLTSAETREWLVTNGIGGYASGTIAGLLTRRYHGLLVAALNPPLGRTLLVTKLDEIVEYHEQVYPLYTNRWSGGAINGYGHHHIEKFHLDRTIPTWKFACADTLLEKRIWMQPEANITYVRYSLTRASGPITLTLKAFVNYRDYHSDTHAWNWKMQAEAVENGLCITAFPQASPIYLLTDRGTVQPTHQWCYNFDLAREHDRGLMDHEDHLHIATFSATLHPGESFCFVASTEPNPGLNAEAALELRRTYERKLLGYWQDSCPSNIREVPEWVQQLVFAADQFVVNRAIPENPIGKTIIAGYPWFGDWGRDTMISLPGITMTTGRLQITRAILYTYSKCVEEGMLPNRFTDTGETPDYNTVDATLWYFEAIRQYYDLTEDIDIVENLFPVLADIIQYHCRGTRYNIHLDPADGLLYAGEQGSQLTWMDAKIGDWVVTPRIGKPVEINALWYNALRIMGKFARQIGRPYQEYDALADRALARFERFWNPDLGYCYDVIDGIYGDDASLRPNQIFAVSLPHSALSPDQQRAVVETCGRYLLTSSGLRSLAPNHFHYRGYYEGNPVQRDSAYHQGTVWAWLLGPFVIAHLRVFQNPTVARSFLEPISNHLTTGGLGTISEIFDGNAPMYPPGLYCPSLVSGRSSAGLVSHRTLDRGSWKIMGLKAVLFDFNGIIINDESIHEQLIAELLLSENLRSKPEEIRKFSLGRSDRACLTDLLTYRGRVVSNTYLNQLIERKSISYRNYLEQLEQLPLYLGLENFLQQLQLADLKLGIVTGALRAEVELVLTRSGLKDYFPVIVAGDDITLSKPEPEGYLLGLQRLNEAYPYLNLQPSECLAVEDTCVGIIAAGRAGMQVAGVAHTYPLHILQRWANWAVDRFSDLELERIQKNFLEEPLQSQV

SEQ ID NO: 369 - protein, 913 aa, synthetic construct
MSINFGREICGDLTSAETREWLVTNGIGGYASGTIAGLLTRRYHGLLVAALNPPLGRTLLVTKLDEIVEYHEQVYPLYTNRWSGGAINGYGHHHIEKFHLDRTIPTWKFACADTLLEKRIWMQPEANITYVRYSLTRASGPITLTLKAFVNYRDYHSDTHAWNWKMQAEAVENGLCITAFPQASPIYLLTDRGTVQPTHQWCYNFDLAREHDRGLMDHEDHLHIATFSATLHPGESFCFVASTEPNPGLNAEAALELRRTYERKLLGYWQDSCPSNIREVPEWVQQLVFAADQFVVNRAIPENPIGKTIIAGYPWFGDWGRDTMISLPGITMTTGRLQITRAILYTYSKCVEEGMLPNRFTDTGETPDYNTVDATLWYFEAIRQYYDLTEDIDIVENLFPVLADIIQYHCRGTRYNIHLDPADGLLYAGEQGSQLTWMDAKIGDWVVTPRIGKPVEINALWYNALRIMGKFARQIGRPYQEYDALADRALARFERFWNPDLGYCYDVIDGIYGDDASLRPNQIFAVSLPHSALSPDQQRAVVETCGRYLLTSSGLRSLAPNHFHYRGYYEGNPVQRDSAYHQGTVWAWLLGPFVIAHLRVFQNPTVARSFLEPISNHLTTGGLGTISEIFDGNAPMYPPGLYCPSLVSGRSSAGLVSHRTLDRGSWKIMGLKAVLFDFNGIIINDESIHEQLIAELLLSENLRSKPEEIRKFSLGRSDRACLTDLLTYRGRVVSNTYLNQLIERKSISYRNYLEQLEQLPLYLGLENFLQQLQLADLKLGIVTGALRAEVELVLTRSGLKDYFPVIVAGDDITLSKPEPEGYLLGLQRLNEAYPYLNLQPSECLAVEDTCVGIIAAGRAGMQVAGVAHTYPLHILQRWANWAVDRFSDLELERIQKNFLEEPLQSQVHHHHHH

SEQ ID NO: 370 - protein, 797 aa, synthetic construct
MPRQADPASPRAPQAAAPASSSARCAAPGAAPPQSAQPPQSAQPPQSAQPPQSAQPPQSAQPPQSPQSPQPAPSTPAFIAPEPDAQKAVRGSQFVLKAGDAFVVSDALGDIAGRDDGLFVDDMRVLSQWRLTFGGRAPSLLSGATSADNASFTAHLTNRPLPPLGGRETPEGVIHIERMRVLADNVLHEALTLTNYGTCDAEVPLSVSFGADFKDMFEVRGSRRERRGAVAPPCVEAGAVRLRYDGLDAVERSVRIGFEPKPDTLAVDRADYTLTIAAQACVSLYLTVEARVGAAHAGGDAFARRPCVATGRGALRKALADVHRAMRERRRTIARVHTSNPLFNAWLDRSLADLGLLTTSLETGPYPYAGIPWFSTPFGRDAVITSLQMLWLQPSLARGVLRFLAEHQARETSAFRDAEPGKIMHEFRKSEMAATGEVPFALYYGGVDTTPLFVVLAGAYLEHTGDESLIDELWPALERAAQWVSTVCERNPRGLLDYQRTSARGLANQGWKDSQDSVFHADGRFPSGPIALVEVQAYACAAFDAMATFSRRRGHAADTVRYAQRAKRLREQVEALFWMPEAGFYGIAVDGHGELCRVLASNAAHLLAFGLPEQSRGESVARVLGSTLFRTGWGVRTLAAGQPRFNPMAYHNGSIWPHDNALAARGLARYGDKRAVLDLLRALFEAAVSFDMRLPELFCGFPRRRGEPPTAYPVACLPQAWAAGAPFMMLQACLGISVDAARGEVRVERPELPEGVDWLRVDDLRVGGDSVSLTFRRVEGQVVAAAEPGGARVVAVL

SEQ ID NO: 371 - protein, 803 aa, synthetic construct
MPRQADPASPRAPQAAAPASSSARCAAPGAAPPQSAQPPQSAQPPQSAQPPQSAQPPQSAQPPQSPQSPQPAPSTPAFIAPEPDAQKAVRGSQFVLKAGDAFVVSDALGDIAGRDDGLFVDDMRVLSQWRLTFGGRAPSLLSGATSADNASFTAHLTNRPLPPLGGRETPEGVIHIERMRVLADNVLHEALTLTNYGTCDAEVPLSVSFGADFKDMFEVRGSRRERRGAVAPPCVEAGAVRLRYDGLDAVERSVRIGFEPKPDTLAVDRADYTLTIAAQACVSLYLTVEARVGAAHAGGDAFARRPCVATGRGALRKALADVHRAMRERRRTIARVHTSNPLFNAWLDRSLADLGLLTTSLETGPYPYAGIPWFSTPFGRDAVITSLQMLWLQPSLARGVLRFLAEHQARETSAFRDAEPGKIMHEFRKSEMAATGEVPFALYYGGVDTTPLFVVLAGAYLEHTGDESLIDELWPALERAAQWVSTVCERNPRGLLDYQRTSARGLANQGWKDSQDSVFHADGRFPSGPIALVEVQAYACAAFDAMATFSRRRGHAADTVRYAQRAKRLREQVEALFWMPEAGFYGIAVDGHGELCRVLASNAAHLLAFGLPEQSRGESVARVLGSTLFRTGWGVRTLAAGQPRFNPMAYHNGSIWPHDNALAARGLARYGDKRAVLDLLRALFEAAVSFDMRLPELFCGFPRRRGEPPTAYPVACLPQAWAAGAPFMMLQACLGISVDAARGEVRVERPELPEGVDWLRVDDLRVGGDSVSLTFRRVEGQVVAAAEPGGARVVAVLHHHHHH

SEQ ID NO: 372 - protein, 742 aa, synthetic construct
MPDLDTREWLLTNGLGSFASGTISDVRTRTYHGWLFTATNPPSGRTLLFSHLDASLEVSGNVVALGTNVWGNGQIELTGYELLRCFDINPVPKWIWGRDNWQLSRQLVMPYGLVEAGDTSTSSVHRWRLGIRNEELEIAKNASQCPMTAVAPLGETPSRSVSQRRPHCLPNAQFCHRILIQYRYDGNDTAILRLRLLIAERDFHHPQIASSGLQFSELLGQQQICLQAKKLGNFGIPWHLRWTKGKYQPDAVWYWNYGLSEETKRGLSDKEDLYSPGYLIVRLQPGDVVTLEARVGFPNLMADVLTSETFAEAVEVEQDRLSQIFGWSEESREQGAREKSFPLYPPHPAPVPLLNAQSLLWQQLLKASDQFIVYRASIAGPTMIAGYHWFNERGRDILIALPGLALVPQRFDLAKGVLRTFRHYYRHGLMPNVFPDADGEPLYNSIDAALWWVETLGLYLEATQDWEFLAEQFSVVQQIHKAFVGGTHYNIQVDAIDGLISWDARGVALTWMDVVIGANPVTPRHGKPVEINALWYSALCWLSRWAERLSSLELGDPVRLTKQAQRYALQAEKVKTSLQKFWNPQLGYLYDTIEPDDRRNFQIRPNAVLALSLHHCGFSAQQGSQILDLATISLLTPYGLRSLDPGDPEYKGRYEGNQEQRDRAYHQGTVWGWLIGPYIRAWQRFYPEQSLPFDWQPLLDHFLSGACLGSISEIFDGDAPHTPRGAIAQAWSVAEIIRHIQK

SEQ ID NO: 373 - protein, 748 aa, synthetic construct
MPDLDTREWLLTNGLGSFASGTISDVRTRTYHGWLFTATNPPSGRTLLFSHLDASLEVSGNVVALGTNVWGNGQIELTGYELLRCFDINPVPKWIWGRDNWQLSRQLVMPYGLVEAGDTSTSSVHRWRLGIRNEELEIAKNASQCPMTAVAPLGETPSRSVSQRRPHCLPNAQFCHRILIQYRYDGNDTAILRLRLLIAERDFHHPQIASSGLQFSELLGQQQICLQAKKLGNFGIPWHLRWTKGKYQPDAVWYWNYGLSEETKRGLSDKEDLYSPGYLIVRLQPGDVVTLEARVGFPNLMADVLTSETFAEAVEVEQDRLSQIFGWSEESREQGAREKSFPLYPPHPAPVPLLNAQSLLWQQLLKASDQFIVYRASIAGPTMIAGYHWFNERGRDILIALPGLALVPQRFDLAKGVLRTFRHYYRHGLMPNVFPDADGEPLYNSIDAALWWVETLGLYLEATQDWEFLAEQFSVVQQIHKAFVGGTHYNIQVDAIDGLISWDARGVALTWMDVVIGANPVTPRHGKPVEINALWYSALCWLSRWAERLSSLELGDPVRLTKQAQRYALQAEKVKTSLQKFWNPQLGYLYDTIEPDDRRNFQIRPNAVLALSLHHCGFSAQQGSQILDLATISLLTPYGLRSLDPGDPEYKGRYEGNQEQRDRAYHQGTVWGWLIGPYIRAWQRFYPEQSLPFDWQPLLDHFLSGACLGSISEIFDGDAPHTPRGAIAQAWSVAEIIRHIQKHHHHHH

SEQ ID NO: 374 - protein, 746 aa, synthetic construct
MGLARDARILKDGELFVVSDERGDLRRTLPGAGVYARDTRFLSELWLLLNGEEPELFDSSAERTNTAIFEFGNAVFRDEEGRDILAHTISLRRCRSVRRGTLDEELVLENYNAFDITVELAFVFAADFLDIFEVRGFPRERPRGRILLPQQQDHSLRLAYRAPDDVLLETEIVFSRQPDTVTTEPGATHLSELALPRMLLPGHDQLIRAPGMIHPPRAFVLFHLKLPSHGRERLALRIAPRELLERRIAVRVPDVSRLTEPVEPPTYSFARARTDHELFDRVLQRSLDDVRALLTPFPGGRLVAAGIPWFVAPFGRDSLIVSLQMMLFAPELALETLRFLARYQGRKHDPWTEEEPGKILHELRFGEMVRLGETPHSPYYGTVDATPLFIVTFCEVMDWVGTPALFDELFPAVQAALHWIDQHGDHNGDGFIDYGRVSPRGLVHQNWKDSHNSLLFPDGRDPVPPIAAVEVQGYVYQAKRALAAVLQRFGKPSHQVLVARLRAEAEQLRARFEQRFWSESDQFYGQAIDGQGRLVPAISSNPGHCLWSEIIGPERAARVVERLFRSDLYSGWGIRTLASTMPHYNPMSYHNGSIWPHDNALIIAGLRRYGYHREALRLASDLLEVAVSFPYYRLPELFCGFGRDEAECMVPISYPVSCSPQAWAAGTMPFLLRIMLGAEARAAERRLTLRPAFPDWLTEVEIRGLRFAGTTLDLAVRRQAGRYTLEVEADPGITVVLAGSSDDFRR

SEQ ID NO: 375 - protein, 752 aa, synthetic construct
MGLARDARILKDGELFVVSDERGDLRRTLPGAGVYARDTRFLSELWLLLNGEEPELFDSSAERTNTAIFEFGNAVFRDEEGRDILAHTISLRRCRSVRRGTLDEELVLENYNAFDITVELAFVFAADFLDIFEVRGFPRERPRGRILLPQQQDHSLRLAYRAPDDVLLETEIVFSRQPDTVTTEPGATHLSELALPRMLLPGHDQLIRAPGMIHPPRAFVLFHLKLPSHGRERLALRIAPRELLERRIAVRVPDVSRLTEPVEPPTYSFARARTDHELFDRVLQRSLDDVRALLTPFPGGRLVAAGIPWFVAPFGRDSLIVSLQMMLFAPELALETLRFLARYQGRKHDPWTEEEPGKILHELRFGEMVRLGETPHSPYYGTVDATPLFIVTFCEVMDWVGTPALFDELFPAVQAALHWIDQHGDHNGDGFIDYGRVSPRGLVHQNWKDSHNSLLFPDGRDPVPPIAAVEVQGYVYQAKRALAAVLQRFGKPSHQVLVARLRAEAEQLRARFEQRFWSESDQFYGQAIDGQGRLVPAISSNPGHCLWSEIIGPERAARVVERLFRSDLYSGWGIRTLASTMPHYNPMSYHNGSIWPHDNALIIAGLRRYGYHREALRLASDLLEVAVSFPYYRLPELFCGFGRDEAECMVPISYPVSCSPQAWAAGTMPFLLRIMLGAEARAAERRLTLRPAFPDWLTEVEIRGLRFAGTTLDLAVRRQAGRYTLEVEADPGITVVLAGSSDDFRRHHHHHH

SEQ ID NO: 376 - protein, 1434 aa, synthetic construct
MNVSEMISQLPRPGTSRVMFCGDVLTFTLSIPSNVSGMAWIRTNLGRASIARNEIIDEVDRNEIKLDEAWHDIQMTRQGPAVFAITLPLSETGHFRAKCFFWAEDSHAPVWPPGEDTIINVEPAGTCCANIVYNAFVRQFGPTRSQGQPLAQEEADCIQCLDKRGYAVIPSSGKFRDLSGEIDFILTHLGCRAIHLLPIHPTPTTYGRMGRFGSPYAALNFTDVDPGLAEFDSSATPMEQFSALVDKIHSCAGYLILDIAINHTGWAASIHEKHPEWLVRNKEGEIAVPGAWGVLWADLTRLDYSKKALWQYMADIFLLWCTRGVDGFRCDAGYMIPVPAWEYIVAKVKKEYPDTLFFLEGLGGPLDATCDILSQANFNWAYSELFQNYDRKAVEAYLPTAISISEKFGHMIHFAETHDNQRLAATSTRYAKMRTSLCALFSVCGGFGFANGVEWFATEKINVHESTCLNWGAKINQTDHIRRLNLILKTHPAFADNVSLDLVQTGQGNYLVLCRRIAATDREIIVVANLDCKMSQTVLWQARHQPEGVLFDLVTEQQVVAKLEGEILSVEVEPGRVMALTRDRQDIKRLQAADLKNDPIPWRVVRQKQVACALKIVTTLAGYGDVSGLDLDYEADALVADPVAFIRSFNPDGNESRVVVWEWNRDVRRQVMVPHGFFLMVVAPANFRAEIRTRTQGTVRTLEQNEALATRDGRFFAVFLPFDTQGRVMEHTLKIRVLGAKGTQKAKAPLVFLPGSGTAFVSSTHTRKAIAKNPSLKLLGVNHRGAMMRAFAWWGRLESRYDALLAANIQPDFPEERWMALTRFRIWATFQGYSRELTLDCLESFTFSYGNRGLWRFRLPTSEGSHCPIEIALKMVDQTNAIEMDITRPAGSPRRNPLGDDKQVTIIIRPDLEHRSFHETVKAWKGPENQWKSSISMVSGGFLFAPDPNHCTLGVKASDGTFVWEPEWQYMVHRSLDAERGLDPDSDLFSPGYFIANLGGRDTIRISARVTCGKGPERDDILTGSDGPDTEFVRGWPLPEALTKSLDAFLVDRAKDRSVIAGYPWFLDWGRDSLIFSRALIQGERLVDAKKILRLFGRFEANGSLPNMISGNDARNRETSDAPLWFIAAAREVVEKEGGALLDQTLGGRTFKEILVSIGDAYVRGTQTGIAMDPETALVFSPSHFTWMDTNFPAGTPREGYPIEIQALWAYSLEFLAKIDPNGTQAWARLADQVKDSIKTLFFMESRGYFSDCLHTRGKAPAGSAVVDDALRPNQLFLISLGVIRDKDLCIPALEHCMELLVPGAIRTLDDSPVDLPLVIEGPTGPLNDPHRPYWGHYMGDEDTRRKPAYHNGTAWTWVFPVFCEAWAETFGSKGAMTGISWLTSSVKLMEQGVAGFVPEIVDGDAPHAPRGCDAQAWGASELYRVWHKLSLRS

SEQ ID NO: 377 - protein, 1440 aa, synthetic construct
MNVSEMISQLPRPGTSRVMFCGDVLTFTLSIPSNVSGMAWIRTNLGRASIARNEIIDEVDRNEIKLDEAWHDIQMTRQGPAVFAITLPLSETGHFRAKCFFWAEDSHAPVWPPGEDTIINVEPAGTCCANIVYNAFVRQFGPTRSQGQPLAQEEADCIQCLDKRGYAVIPSSGKFRDLSGEIDFILTHLGCRAIHLLPIHPTPTTYGRMGRFGSPYAALNFTDVDPGLAEFDSSATPMEQFSALVDKIHSCAGYLILDIAINHTGWAASIHEKHPEWLVRNKEGEIAVPGAWGVLWADLTRLDYSKKALWQYMADIFLLWCTRGVDGFRCDAGYMIPVPAWEYIVAKVKKEYPDTLFFLEGLGGPLDATCDILSQANFNWAYSELFQNYDRKAVEAYLPTAISISEKFGHMIHFAETHDNQRLAATSTRYAKMRTSLCALFSVCGGFGFANGVEWFATEKINVHESTCLNWGAKINQTDHIRRLNLILKTHPAFADNVSLDLVQTGQGNYLVLCRRIAATDREIIVVANLDCKMSQTVLWQARHQPEGVLFDLVTEQQVVAKLEGEILSVEVEPGRVMALTRDRQDIKRLQAADLKNDPIPWRVVRQKQVACALKIVTTLAGYGDVSGLDLDYEADALVADPVAFIRSFNPDGNESRVVVWEWNRDVRRQVMVPHGFFLMVVAPANFRAEIRTRTQGTVRTLEQNEALATRDGRFFAVFLPFDTQGRVMEHTLKIRVLGAKGTQKAKAPLVFLPGSGTAFVSSTHTRKAIAKNPSLKLLGVNHRGAMMRAFAWWGRLESRYDALLAANIQPDFPEERWMALTRFRIWATFQGYSRELTLDCLESFTFSYGNRGLWRFRLPTSEGSHCPIEIALKMVDQTNAIEMDITRPAGSPRRNPLGDDKQVTIIIRPDLEHRSFHETVKAWKGPENQWKSSISMVSGGFLFAPDPNHCTLGVKASDGTFVWEPEWQYMVHRSLDAERGLDPDSDLFSPGYFIANLGGRDTIRISARVTCGKGPERDDILTGSDGPDTEFVRGWPLPEALTKSLDAFLVDRAKDRSVIAGYPWFLDWGRDSLIFSRALIQGERLVDAKKILRLFGRFEANGSLPNMISGNDARNRETSDAPLWFIAAAREVVEKEGGALLDQTLGGRTFKEILVSIGDAYVRGTQTGIAMDPETALVFSPSHFTWMDTNFPAGTPREGYPIEIQALWAYSLEFLAKIDPNGTQAWARLADQVKDSIKTLFFMESRGYFSDCLHTRGKAPAGSAVVDDALRPNQLFLISLGVIRDKDLCIPALEHCMELLVPGAIRTLDDSPVDLPLVIEGPTGPLNDPHRPYWGHYMGDEDTRRKPAYHNGTAWTWVFPVFCEAWAETFGSKGAMTGISWLTSSVKLMEQGVAGFVPEIVDGDAPHAPRGCDAQAWGASELYRVWHKLSLRSHHHHHH

SEQ ID NO: 378 - protein, 706 aa, synthetic construct
MDYRVIKENDLFLLTDPAGNINENHQYGLGLYMKDTRFLSKFRLKINDADPVLLSSSAEENLLATILLTNPHMEKEGELVLWRESIEIERKRFIYGGVFYEKIKLKSYFPKKIAFTLSIQADVDFNDMFIVRGFQTGNVGKRTGEIKGESSLTYTYKGADDIKRATRILWDKEASYVDEQGEVTFSFELNHQEEEEITLMVQPQMGEDINENILSADEALSLLTQSYKKWEKSSTKVTTDHPTMQRLITRGLNDLRVLLTDVGFGAFPVAGLPWFGVPFGRDSLIAALQMLAFNPEVAKGTLRTMAAYQGTANDPWRDEQPGKIMHEIRYGELANTNQIPFTPYYGTVDATPLFLILLTEYVKWTGDLEFVQEMHMTIDRALTWINDYGDRDGDLFLEYHQESSKGIANQGWKDSGDSVVHRTGEYAKTPIALVEVQGYVYQAKMGIASLYDQLNKTDEANNLRKEARQLRKKFNEAFWMDDVSFYAIALDEHKSQVGTITSNPGHALLAEIMDEEKLHKVSERLISDSMFSGYGIRTMAEGEAGYNPMSYHDGSIWPHDNSLIVLGLSKANKQTEANKVIKGLVDAAEHFEYDRLPELFCGYSSKLDKPVKYPVACSPQAWAAGTPLAFVQAMLRLFPDSTAGTIQINPVLIDDMNELSVENLVIGSGKLSLTVKKEESETIIYVKENTTKYELLSQCKVRNYVG

SEQ ID NO: 379 - protein, 712 aa, synthetic construct
MDYRVIKENDLFLLTDPAGNINENHQYGLGLYMKDTRFLSKFRLKINDADPVLLSSSAEENLLATILLTNPHMEKEGELVLWRESIEIERKRFIYGGVFYEKIKLKSYFPKKIAFTLSIQADVDFNDMFIVRGFQTGNVGKRTGEIKGESSLTYTYKGADDIKRATRILWDKEASYVDEQGEVTFSFELNHQEEEEITLMVQPQMGEDINENILSADEALSLLTQSYKKWEKSSTKVTTDHPTMQRLITRGLNDLRVLLTDVGFGAFPVAGLPWFGVPFGRDSLIAALQMLAFNPEVAKGTLRTMAAYQGTANDPWRDEQPGKIMHEIRYGELANTNQIPFTPYYGTVDATPLFLILLTEYVKWTGDLEFVQEMHMTIDRALTWINDYGDRDGDLFLEYHQESSKGIANQGWKDSGDSVVHRTGEYAKTPIALVEVQGYVYQAKMGIASLYDQLNKTDEANNLRKEARQLRKKFNEAFWMDDVSFYAIALDEHKSQVGTITSNPGHALLAEIMDEEKLHKVSERLISDSMFSGYGIRTMAEGEAGYNPMSYHDGSIWPHDNSLIVLGLSKANKQTEANKVIKGLVDAAEHFEYDRLPELFCGYSSKLDKPVKYPVACSPQAWAAGTPLAFVQAMLRLFPDSTAGTIQINPVLIDDMNELSVENLVIGSGKLSLTVKKEESETIIYVKENTTKYELLSQCKVRNYVGHHHHHH

SEQ ID NO: 380 - protein, 686 aa, synthetic construct
MADFIRKITRDALRSAPDAALSREWLVANGLGGYASSSLSGAVTRRYHGLLVAALPAPLGRVVMLSQLNDVLIERDGTERRLTGHNPQADQWPDPNYPSDVAPSGLAEFRMESGLPVWRYEIGDVVIEKQIVLPHLQNIVHVTYRVVKAPQPICLRLRPVMAFRKLESAVDEPLARDYRVTARGHEYEIWAGPELPVLRMTIEGAELPLTLDGGARRDIVYPVEAERGYESRGSLWTPGYFSGPLSQGQTLSFVAATEPWQRLWALPPADVLRFEKERRRRIVGTAHPSLREGAMAELVLSADSFLFVPAGRVADQMRARAEGDEVRTVIAGYHWFTDWGRDTMISLEGLTLTTGRHMEAGWILRTFAHYIRDGLIPNMFPDGKDEGLYHTADATLWFFHALNRYVQATGDRHTLQLLLPRLVEVIDHHRRGTRFGIAVDPGDGLLRQGREGYQLTWMDAKVEDWVVTPRRGKAVEINALWYNALCLTAGWLTEEGDAQAARPLAELAEQARVSFNARFWCAHAGHLYDVVDGERGDDDACRPNQIFAVSLDNPVLDRSRWEPVVETVRQRLLTPVGLRSLAPGQRDYKPKYFGDLRARDAAYHQGTVWGWLIGPYVDAWLKLHPEEKAGARELLQGFLPHLDQAGIGTISEIFDAEPPFSPRGCISQAWSVAEVLRCWAATAEDR

SEQ ID NO: 381 - protein, 692 aa, synthetic construct
MADFIRKITRDALRSAPDAALSREWLVANGLGGYASSSLSGAVTRRYHGLLVAALPAPLGRVVMLSQLNDVLIERDGTERRLTGHNPQADQWPDPNYPSDVAPSGLAEFRMESGLPVWRYEIGDVVIEKQIVLPHLQNIVHVTYRVVKAPQPICLRLRPVMAFRKLESAVDEPLARDYRVTARGHEYEIWAGPELPVLRMTIEGAELPLTLDGGARRDIVYPVEAERGYESRGSLWTPGYFSGPLSQGQTLSFVAATEPWQRLWALPPADVLRFEKERRRRIVGTAHPSLREGAMAELVLSADSFLFVPAGRVADQMRARAEGDEVRTVIAGYHWFTDWGRDTMISLEGLTLTTGRHMEAGWILRTFAHYIRDGLIPNMFPDGKDEGLYHTADATLWFFHALNRYVQATGDRHTLQLLLPRLVEVIDHHRRGTRFGIAVDPGDGLLRQGREGYQLTWMDAKVEDWVVTPRRGKAVEINALWYNALCLTAGWLTEEGDAQAARPLAELAEQARVSFNARFWCAHAGHLYDVVDGERGDDDACRPNQIFAVSLDNPVLDRSRWEPVVETVRQRLLTPVGLRSLAPGQRDYKPKYFGDLRARDAAYHQGTVWGWLIGPYVDAWLKLHPEEKAGARELLQGFLPHLDQAGIGTISEIFDAEPPFSPRGCISQAWSVAEVLRCWAATAEDRHHHHHH

SEQ ID NO: 382 - protein, 736 aa, synthetic construct
MTNRIALRARANQFFVYSGRSLLVTNLDGDVSLSGTNGFYRENTRLLSRYELTVNGYRLVPVVASPVGGAGMLTYQQMPDEVGVPERSVAVEIAYFLSEGMRTVLRIENNWGVKSHQPDPPTIDLAINVAADFADISEADTGTRLQKAPVQASWDDTRRELCFRYCHPKLDRAVAIRVEQSPVPVRYEQDALIVSLTLALHQPVELVLVVEPVFDGKRLTAPPPVYDETETGLGRLRQRLRDETPRLTTSNATVARAWETAVSDLATLPLGRDPGPAAPIAGLPLYQQFFGRDTLTIGWQSLIAMPAMLRDALRLNAAWQGTVIDDFYDEEPGKLIHQASRAPLAILGYNPFLHYYGDYATPPDFLLGLGQYAAWTSDRETVRPLLENARKAIDWLDRYGDLDHDGFLEYVCRSPEGLKNQGWKDSWDGIIDQDGNVVPDPIAACELQGYWYVGLQQVAIAFLLAGDLPYALELLNKARDLKARFDPAFWMEDEGFYALGLGPDKEQIRSIASNAGHLLAAGIVPAEKAPRVARRLMEPDMFSGWGIRTLSSHHVAYNPFSYHLGSVWPVESGTIAFGMARYSCWPECHRLAGGLFALTERCSESRLPEVVGGIQRDEAHPYPGIYPGSNEPQGWSDGAIVVTIQALLGMHGLAPLGLLLVDPHLPDWLPDLRLNGLRVGESELDLDFVRTKSGKTRYHVRRQQGHIRVVHQPVPSEPHATLIGRLLDALGSIGHS

SEQ ID NO: 383 - protein, 742 aa, synthetic construct
MTNRIALRARANQFFVYSGRSLLVTNLDGDVSLSGTNGFYRENTRLLSRYELTVNGYRLVPVVASPVGGAGMLTYQQMPDEVGVPERSVAVEIAYFLSEGMRTVLRIENNWGVKSHQPDPPTIDLAINVAADFADISEADTGTRLQKAPVQASWDDTRRELCFRYCHPKLDRAVAIRVEQSPVPVRYEQDALIVSLTLALHQPVELVLVVEPVFDGKRLTAPPPVYDETETGLGRLRQRLRDETPRLTTSNATVARAWETAVSDLATLPLGRDPGPAAPIAGLPLYQQFFGRDTLTIGWQSLIAMPAMLRDALRLNAAWQGTVIDDFYDEEPGKLIHQASRAPLAILGYNPFLHYYGDYATPPDFLLGLGQYAAWTSDRETVRPLLENARKAIDWLDRYGDLDHDGFLEYVCRSPEGLKNQGWKDSWDGIIDQDGNVVPDPIAACELQGYWYVGLQQVAIAFLLAGDLPYALELLNKARDLKARFDPAFWMEDEGFYALGLGPDKEQIRSIASNAGHLLAAGIVPAEKAPRVARRLMEPDMFSGWGIRTLSSHHVAYNPFSYHLGSVWPVESGTIAFGMARYSCWPECHRLAGGLFALTERCSESRLPEVVGGIQRDEAHPYPGIYPGSNEPQGWSDGAIVVTIQALLGMHGLAPLGLLLVDPHLPDWLPDLRLNGLRVGESELDLDFVRTKSGKTRYHVRRQQGHIRVVHQPVPSEPHATLIGRLLDALGSIGHSHHHHHH

SEQ ID NO: 384 - protein, 868 aa, synthetic construct
MSIADYLSQVPLFQGLSACERVKLAESMRRHTYAPNDVIVRQGDKTTDFYIILSGSVRVSRKAGRREIPIAELHAGDFFGDMALLDSLPRSATVTAIEPTECLSLSWEAFHRDVLGKPEVARAMLSTLSRRVRDVEDLAFQPQTQSPATAREREDRLLREGESSLITDIRETLTIKSGNLFLLCDEEGNIPIGNTAGLGLYLGDTRHLSGYEMILDQIRPTTLLTSARLGSASEQQLTNRDIRIRNRIVRKETLLISRSRVIGSGLEERLVVSNFNSFEVTITLLLRFASDFADIFEVRGLRRDQRGSIHRPTVDPVGVTLSYDGLDHVRRETRLEFSPAPDRVSGTSAVFRLRLPPLGSVSLRITVTASHSSLHPVARDQSRPLMPPPGQGAGWLAAQTNIETNNEFFNAALDRSLADLRLLINRLDDDEYIAAGTPWYASLFGRDSLITAWQTLAWNPEIARGVLRLLARFQGRQRDDWRDEEPGKILHELRTGELAKSGLIPHSPYYGAVDSTPLFVMLLGAYERWTGDQALVEQLAPALDAALQWMIQDGDPDHDGFIEYSRRSIKGLTNQGWKDSGEGIVNRNGSLPQPPIALVEVQGYAYAAYLAGADLLETLGRTGEAGNLRARAVLLRDQFEQQFWMETERYYALGLDGRKQHITAITSNPGHALWTGIVSQERAELVADRLLSPALFSGWGVRTLAEDMAAYNPLGYHLGTIWPHDNALIVAGLMRYGCSDHARRIATSLFDAGRAFRYYRLPELFCGITRTEHSTPIGYPVACSPQAWAAGSLPYILQALLGIHPQSSGRVLLVDHPALPDWLREVRVNRLRIGSGRVNLVARREGPRTAVDVLAVDGDVAVDVRLGG

SEQ ID NO: 385 - protein, 874 aa, synthetic construct
MSIADYLSQVPLFQGLSACERVKLAESMRRHTYAPNDVIVRQGDKTTDFYIILSGSVRVSRKAGRREIPIAELHAGDFFGDMALLDSLPRSATVTAIEPTECLSLSWEAFHRDVLGKPEVARAMLSTLSRRVRDVEDLAFQPQTQSPATAREREDRLLREGESSLITDIRETLTIKSGNLFLLCDEEGNIPIGNTAGLGLYLGDTRHLSGYEMILDQIRPTTLLTSARLGSASEQQLTNRDIRIRNRIVRKETLLISRSRVIGSGLEERLVVSNFNSFEVTITLLLRFASDFADIFEVRGLRRDQRGSIHRPTVDPVGVTLSYDGLDHVRRETRLEFSPAPDRVSGTSAVFRLRLPPLGSVSLRITVTASHSSLHPVARDQSRPLMPPPGQGAGWLAAQTNIETNNEFFNAALDRSLADLRLLINRLDDDEYIAAGTPWYASLFGRDSLITAWQTLAWNPEIARGVLRLLARFQGRQRDDWRDEEPGKILHELRTGELAKSGLIPHSPYYGAVDSTPLFVMLLGAYERWTGDQALVEQLAPALDAALQWMIQDGDPDHDGFIEYSRRSIKGLTNQGWKDSGEGIVNRNGSLPQPPIALVEVQGYAYAAYLAGADLLETLGRTGEAGNLRARAVLLRDQFEQQFWMETERYYALGLDGRKQHITAITSNPGHALWTGIVSQERAELVADRLLSPALFSGWGVRTLAEDMAAYNPLGYHLGTIWPHDNALIVAGLMRYGCSDHARRIATSLFDAGRAFRYYRLPELFCGITRTEHSTPIGYPVACSPQAWAAGSLPYILQALLGIHPQSSGRVLLVDHPALPDWLREVRVNRLRIGSGRVNLVARREGPRTAVDVLAVDGDVAVDVRLGGHHHHHH

SEQ ID NO: 386 - protein, 753 aa, synthetic construct
MTPDTLMTPETIVLDNRTFTPADQKEISEWPCVVSQRPQPTITVKDDDLFLVTDTMGNISDCYLYGSTPSMGMFCRDTRFLSRLELQIEGRSPILLSSNADKGFALSVLCTNPDLENLFKADTVGIRREIVLNGALFEEIEVANYNTTSIVFELSLSFDADFVDLFEVRGHHRQKRGRLLHLNELTSDHVASSSPQTHKEESLTLAYQGLDASIMESLIQFQHLQPTRFHGSTAVWRIELASHETKKLGYRLNMFTDNQPSSTISAASTLAQAKAAELMEEQNWLQQITRISSDKSLFNRVISRAEQDIYLLLQSFGKHKTVSAGVPWFSTLFGRDSIITASQTLMLNSKIAKETLILLAEYQGQTDNIWREEEPGKILHELRLGEMARCQEIPHTPYYGTVDATPLWLMLYAEYYSWTHDQETLEALWPNALAAMGWIDRQLQETGYLSYARKSKGGLINQGWKDSGDCIVNRKGELAIGPISLCEVQAYVYAAKNRLAEIAKIQQRPDLEELWQTEARQLRAKFNQDFWLEDQDFCALALDGEGKPVDSISSNPGQCLNLGIFTSAKAYSVAERLQAADMFNGWGIRTLSSLSPAYNPMGYHTGSVWPHDNSMIAMGLRSLGLIDQSLELFQGLFNMTSQQPYQRPPELFCGYEMNGDNAPVQYPVACTPQAWATGSIFQLLQMMVNLVPDAQNNCLRIIDPALPESINNLSFHNLRVGGTILDLEFERIGSTTACRVAKKRGNLRVVIEA

SEQ ID NO: 387 - protein, 759 aa, synthetic construct
MTPDTLMTPETIVLDNRTFTPADQKEISEWPCVVSQRPQPTITVKDDDLFLVTDTMGNISDCYLYGSTPSMGMFCRDTRFLSRLELQIEGRSPILLSSNADKGFALSVLCTNPDLENLFKADTVGIRREIVLNGALFEEIEVANYNTTSIVFELSLSFDADFVDLFEVRGHHRQKRGRLLHLNELTSDHVASSSPQTHKEESLTLAYQGLDASIMESLIQFQHLQPTRFHGSTAVWRIELASHETKKLGYRLNMFTDNQPSSTISAASTLAQAKAAELMEEQNWLQQITRISSDKSLFNRVISRAEQDIYLLLQSFGKHKTVSAGVPWFSTLFGRDSIITASQTLMLNSKIAKETLILLAEYQGQTDNIWREEEPGKILHELRLGEMARCQEIPHTPYYGTVDATPLWLMLYAEYYSWTHDQETLEALWPNALAAMGWIDRQLQETGYLSYARKSKGGLINQGWKDSGDCIVNRKGELAIGPISLCEVQAYVYAAKNRLAEIAKIQQRPDLEELWQTEARQLRAKFNQDFWLEDQDFCALALDGEGKPVDSISSNPGQCLNLGIFTSAKAYSVAERLQAADMFNGWGIRTLSSLSPAYNPMGYHTGSVWPHDNSMIAMGLRSLGLIDQSLELFQGLFNMTSQQPYQRPPELFCGYEMNGDNAPVQYPVACTPQAWATGSIFQLLQMMVNLVPDAQNNCLRIIDPALPESINNLSFHNLRVGGTILDLEFERIGSTTACRVAKKRGNLRVVIEAHHHHHH

SEQ ID NO: 388 - protein, 655 aa, synthetic construct
MKISLSKANFRDMNRLEESNYLLTNGLGGYSSLTLAGSCSRNDHALFMGSLVAPNSRFCFVRHLDDRLTLANGTSHPLSSQAKVNPYDTVDSSLLFENFSQTYLPQYTYRIDGIEITKTIVMAQGVNRLGVEYRVFNPLKHPFTLDITPKYHFCAKGEALSKEQAFIHQEGKVSSNGQTLHYRHNGSNTALSAPSYESGLYFSYDARDGRSASDAQVALHTITFDGTESNESFFVIFEAEEERAHYLAPSSIETMINDEVQRQQACITQSGLKSTFAQQLVRASDQFVVMRDSTESKSIIAGYPFFADWGRDTMIALWGCNLSTNQFEDAKQIFRTFIRYEHKGILPNLFPEAGVEPMYNTIDAALLFVVSLYEYYQASNDLDFIRDEAYDCMVNIYQHYRNGTDFDIKMLDNGLISGGSGLDQLTWMDVRYGDILPTPRQGCAVEINALWYNALSILHFFNQKLGKSMPELPALTEQVKETFNHAFYNDKGYLNDWIKGEEVNQQIRPNQAYAVGLPFPILSEEKARSVILNIYQHLYTPLGLRSLAMQDPEFKSVHTGSHYDRDMAYHQGTVWGFMIGQFLMGILRHFPNDASLMHIVEQFISHNQDALSEGCIGQLAEIYDGYSPNQSRGCFAQAWSVSEVLRVLKHYEERI

SEQ ID NO: 389 - protein, 661 aa, synthetic construct
MKISLSKANFRDMNRLEESNYLLTNGLGGYSSLTLAGSCSRNDHALFMGSLVAPNSRFCFVRHLDDRLTLANGTSHPLSSQAKVNPYDTVDSSLLFENFSQTYLPQYTYRIDGIEITKTIVMAQGVNRLGVEYRVFNPLKHPFTLDITPKYHFCAKGEALSKEQAFIHQEGKVSSNGQTLHYRHNGSNTALSAPSYESGLYFSYDARDGRSASDAQVALHTITFDGTESNESFFVIFEAEEERAHYLAPSSIETMINDEVQRQQACITQSGLKSTFAQQLVRASDQFVVMRDSTESKSIIAGYPFFADWGRDTMIALWGCNLSTNQFEDAKQIFRTFIRYEHKGILPNLFPEAGVEPMYNTIDAALLFVVSLYEYYQASNDLDFIRDEAYDCMVNIYQHYRNGTDFDIKMLDNGLISGGSGLDQLTWMDVRYGDILPTPRQGCAVEINALWYNALSILHFFNQKLGKSMPELPALTEQVKETFNHAFYNDKGYLNDWIKGEEVNQQIRPNQAYAVGLPFPILSEEKARSVILNIYQHLYTPLGLRSLAMQDPEFKSVHTGSHYDRDMAYHQGTVWGFMIGQFLMGILRHFPNDASLMHIVEQFISHNQDALSEGCIGQLAEIYDGYSPNQSRGCFAQAWSVSEVLRVLKHYEERIHHHHHH

SEQ ID NO: 390 - protein, 717 aa, synthetic construct
MSPRTPKKTRSVAPSRIREGMPFPLGATWDGLGVNFALFSANATKVELCLFDSTGEQELERIELPEYTDEIYHGYLPDAHPGLVYGYRVYGPYEPENGHRFNPNKLLIDPYAKQLVGSLKWSEALFGYTIGHPDGDLSFDERDSAPFVPKCKVIDPAFTWGRDQRVQIPWERTIIYEAHARGISMRHPAVPEELRGTFAGLANDELLKHIKDLGVSSIELLPIHAFVNDQHLLDKGLNNYWGYNSIAFFAPHPRYLASGKIAEFKEMVAHLHDAGLEVILDVVYNHTAEGNERGPTLSMRGIDNASYYRLMPDDKRYYINDSGTGNTLDLSHPCVLQLVTDSLRYWAGEMHVDGFRFDLATILGRYHDGYSERHGFLVACRQDPMLSQVKLIAEPWDCGPGGYQVGNFAPGWAEWNDRFRDTARAFWKGDEGQLADFAARLTASGDMFNNRGRRPYSSVNFITAHDGFTLRDLVSYNHKHNEDNDENNQDGTDNNLSWNCGVEGPTDDPAINALRMRQMRNYFATLLLAQGTPMIVAGDEFSRTQHGNNNAYCQDSEIGWVNWDLDEEGQELLAFVKRLTRLRLAYPVLRRSRFLVGDYNEAIGVKDVTWLAPDGSEMSVEQWEDPHGRCLGMLIDGRAQVSGIARPGAEATVLLIVNAHHDIVPFKLPAVPEGDYWSCLVDTDRPELRKGQHLQFDSTFEVKGRSMLLMVLQYDEE

SEQ ID NO: 391 - protein, 723 aa, synthetic construct
MSPRTPKKTRSVAPSRIREGMPFPLGATWDGLGVNFALFSANATKVELCLFDSTGEQELERIELPEYTDEIYHGYLPDAHPGLVYGYRVYGPYEPENGHRFNPNKLLIDPYAKQLVGSLKWSEALFGYTIGHPDGDLSFDERDSAPFVPKCKVIDPAFTWGRDQRVQIPWERTIIYEAHARGISMRHPAVPEELRGTFAGLANDELLKHIKDLGVSSIELLPIHAFVNDQHLLDKGLNNYWGYNSIAFFAPHPRYLASGKIAEFKEMVAHLHDAGLEVILDVVYNHTAEGNERGPTLSMRGIDNASYYRLMPDDKRYYINDSGTGNTLDLSHPCVLQLVTDSLRYWAGEMHVDGFRFDLATILGRYHDGYSERHGFLVACRQDPMLSQVKLIAEPWDCGPGGYQVGNFAPGWAEWNDRFRDTARAFWKGDEGQLADFAARLTASGDMFNNRGRRPYSSVNFITAHDGFTLRDLVSYNHKHNEDNDENNQDGTDNNLSWNCGVEGPTDDPAINALRMRQMRNYFATLLLAQGTPMIVAGDEFSRTQHGNNNAYCQDSEIGWVNWDLDEEGQELLAFVKRLTRLRLAYPVLRRSRFLVGDYNEAIGVKDVTWLAPDGSEMSVEQWEDPHGRCLGMLIDGRAQVSGIARPGAEATVLLIVNAHHDIVPFKLPAVPEGDYWSCLVDTDRPELRKGQHLQFDSTFEVKGRSMLLMVLQYDEEHHHHHH

SEQ ID NO: 392 - protein, 1534 aa, synthetic construct
MSAHRTLLLRLSDSGEPVTSCSYGQGVLTLPSLPLPQGKKLGDMPVYTVKLAIPAGSPVTRDGLIWTNCPPDFSTQFDREKFYKKIIKTSFHEDDHIDLDIYVPGTYCFYLSFKNDKDELETTRKFYFVVLPILSVNDKFIPLNSIAMQSVVSKWMGPTIKDWEKVFARVASKKYNMIHFTPLQHRGESNSPYSIYDQLEFDPTVFKSEKEVADMVERLRTEHNILSLTDIVFNHTANNSQWLLDHPEAGYNHKTSPHLISAIELDKKLLDFSEQMEALGYPVDLKTVDDLIKVMDGIKEHVIGELKLWEFYVVDVKQTVSELREKWGNSKSWSDDNIPSKDDSTNLAQFVRDNATEPGFGSLGERGSNKINIDKFAAILKKLHSEDYNNGIEELATKILNDINLPFYKEYDDDINEVLEQLFNRIKYLRIDDHGPKQGPITKKLPLSEPYFTRFKAKDGEEYALANNGWIWDGNPLVDFASSQSKAYLRREVIVWGDCVKLRYGKGPSDSPYLWERMSKYVEMNARIFNGFRIDNCHSTPLHVGQYFLDVARRVNPNLYVVAELFSGSEAMDCLFVERLGISSLIREAMQAWSEEELSRLVHRHGGRPIGSYKFVPLDDFPYPADVKIDEEYCAYNPDDHSVKCVSEIMIPKTLTATPPHALFMDCTHDNETPNQKRTVEDTLPNAALVAFCSSAIGSVYGYDEVFPQLLDLVQEKRTYSCAENTGISKVKTLLNNMREEIASEAVDIEDSEMHVHHDGQYITFHRTNAKNGKGWYLVARTKFHSSGDQMLPRIKLSQTKATFKAAFSLERTGDAPISDEIIEGIPTKLRELTGFDIGFDENTKETSILLPQDFPQGSIVIFETQQLGIDDSLDHFIRSGAIKATEKLSLESINYVLYRAEQEEYDYSEGRSGAYDIPDYGKPVYCGLQGWVSILRKIIFYNDLAHPLSNNLRNGHWAVDYVVNRLDLYKDKEGVAEVQEWLRSRMERIKQLPSYLVPSFFALVVGIMYGCCRLRAMQLMSDNVGKSTVFVQSLAMTSIQMVSAMKSTSILPDQNIAAMAAGLPHFSTNYMRCWGRDVFISLRGLLLTTGRYEEAKEHILAFAKTLKHGLIPNLLDAGRNPRYNARDAAWFFVQAIQDYVTIVPGGVSLLQEKVTRRFPLDDEYIPYDDPKAFSYSSTIEEIIYEILNRHAGGIKYREANAGPNLDRVMKDEGFNVEVNVDWETGLIHGGSQFNCGTWMDKMGESEKANSVGVPGTPRDGAAVEINGLLKSCLRFVLQLSKDGKFKYTEVTKPDGSKISLSSWNDLLQENFERCFYVPKNKEDDNKFEIDATIINRRGIYKDLYRSGKPYEDYQFRPNFTIAMVVAPELFTPDYAAGAIELADQVLRGPVGMRTLDPSDYNYRPYYNNGEDSDDFATSKGRNYHQGPEWVWCYGYFIRAYHYFNFLTNPKCQVEGSAKKLKPSSYLYRKLYSRLLKHREWIENSPWAGLAELTNKDGEVCNDSSPTQAWSTGCLLDLFYDLWISYEEHHHHHH

SEQ ID NO: 393 - protein, 484 aa, synthetic construct
MANLNGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 394 - protein, 490 aa, synthetic construct
MANLNGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQRHHHHHH

SEQ ID NO: 395 - protein, 439 aa, synthetic construct
MATSDDWKSKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSA

SEQ ID NO: 396 - protein, 445 aa, synthetic construct
MATSDDWKSKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSAHHHHHH

SEQ ID NO: 397 - protein, 471 aa, synthetic construct
ATPDEWRSRSIYQVLTDRFARGDGSPDAPCDTGARKYCGGNYRGLISQLDYIQGMGFDSVWISPITKQFEDDWNGAPYHGYWQTDLYALNEHFGTEEDLRALADELHARGMFLMVDVVINHNGWPGDAASIDYSQFNPFNSSDYYHPPCEINYDDQTSVEQCWLYTGANALPDLKTEDPHVSQVHNDWIADLVSKYSIDGLRIDTTKHVDKPAIGSFNDAAGVYAVGEVYHGDPAYTCPYQDWVDGVLNFPVYYPLIDAFKSPSGTMWSLVDNINKVFQTCNDPRLLGTFSENHDIPRFASYTQDLALAKNVLAFTILFDGIPIVYAGQEQQYSGDSDPYNREALWLSGFNTDAPLYKHIAACNRIRSHAVSNDDAYITTPTDIKYSDDHTLALVKGAVTTVLTNAGANAGETTVTVEATGYASGEQVTDVLSCESIAASDGGRLSVTLNQGLPRVFFPTDALAGSGLCEN

SEQ ID NO: 398 - protein, 477 aa, synthetic construct
ATPDEWRSRSIYQVLTDRFARGDGSPDAPCDTGARKYCGGNYRGLISQLDYIQGMGFDSVWISPITKQFEDDWNGAPYHGYWQTDLYALNEHFGTEEDLRALADELHARGMFLMVDVVINHNGWPGDAASIDYSQFNPFNSSDYYHPPCEINYDDQTSVEQCWLYTGANALPDLKTEDPHVSQVHNDWIADLVSKYSIDGLRIDTTKHVDKPAIGSFNDAAGVYAVGEVYHGDPAYTCPYQDWVDGVLNFPVYYPLIDAFKSPSGTMWSLVDNINKVFQTCNDPRLLGTFSENHDIPRFASYTQDLALAKNVLAFTILFDGIPIVYAGQEQQYSGDSDPYNREALWLSGFNTDAPLYKHIAACNRIRSHAVSNDDAYITTPTDIKYSDDHTLALVKGAVTTVLTNAGANAGETTVTVEATGYASGEQVTDVLSCESIAASDGGRLSVTLNQGLPRVFFPTDALAGSGLCENHHHHHH

SEQ ID NO: 399 - protein, 593 aa, synthetic construct
MEEHTQGSHVEAGIVEHPNAEDFGHARTLPTDTNWFKHAVFYEVLVRAFYDSNADGIGDLRGLTEKLDYIKWLGVDCLWLPPFYDSPLRDGGYDIRDFYKVLPEFGTVDDFVTLLDAAHRRGIRIITDLVMNHTSDQHEWFQESRHNPDGPYGDFYVWSDTSDRYPDARIIFVDTEESNWTFDPVRRQFYWHRFFSHQPDLNYDNPAVQEAMLDVLRFWLDLGIDGFRLDAVPYLFEREGTNCENLPETHAFLKRCRKAIDDEYPGRVLLAEANQWPADVVAYFGDPDTGGDECHMAFHFPLMPRIFMAVRRESRFPISEILAQTPPIPDTAQWGIFLRNHDELTLEMVTDEERDYMYAEYAKDPRMKANVGIRRRLAPLLENDRNQIELFTALLLSLPGSPVLYYGDEIGMGDIIWLGDRDSVRTPMQWTPDRNAGFSKATPGRLYLPPNQDAVYGYHSVNVEAQLDSSSSLLNWTRNMLAVRSRHDAFAVGTFRELGGSNPSVLAYIREVTRQQGDGGAKTDAVLCVNNLSRFPQPIELNLQQWAGYIPVEMTGYVEFPSIGQLPYLLTLPGHGFYWFQLREPDPEPGAQQ

SEQ ID NO: 400 - protein, 598 aa, synthetic construct
EEHTQGSHVEAGIVEHPNAEDFGHARTLPTDTNWFKHAVFYEVLVRAFYDSNADGIGDLRGLTEKLDYIKWLGVDCLWLPPFYDSPLRDGGYDIRDFYKVLPEFGTVDDFVTLLDAAHRRGIRIITDLVMNHTSDQHEWFQESRHNPDGPYGDFYVWSDTSDRYPDARIIFVDTEESNWTFDPVRRQFYWHRFFSHQPDLNYDNPAVQEAMLDVLRFWLDLGIDGFRLDAVPYLFEREGTNCENLPETHAFLKRCRKAIDDEYPGRVLLAEANQWPADVVAYFGDPDTGGDECHMAFHFPLMPRIFMAVRRESRFPISEILAQTPPIPDTAQWGIFLRNHDELTLEMVTDEERDYMYAEYAKDPRMKANVGIRRRLAPLLENDRNQIELFTALLLSLPGSPVLYYGDEIGMGDIIWLGDRDSVRTPMQWTPDRNAGFSKATPGRLYLPPNQDAVYGYHSVNVEAQLDSSSSLLNWTRNMLAVRSRHDAFAVGTFRELGGSNPSVLAYIREVTRQQGDGGAKTDAVLCVNNLSRFPQPIELNLQQWAGYIPVEMTGYVEFPSIGQLPYLLTLPGHGFYWFQLREPDPEPGAQQHHHHHH

SEQ ID NO: 401 - protein, 512 aa, synthetic construct
MKQQKRLYARLLTLLFALIFLLPHSAAAAANLNGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEHRIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNSTVVSKHPLKAVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 402 - protein, 490 aa, synthetic construct
AANLNGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEHRIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNSTVVSKHPLKAVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQRHHHHHH

SEQ ID NO: 403 - protein, 1196 aa, synthetic construct
MTLYRSLWKKGCMLLLSLVLSLTAFIGSPSNTASAAVADDFQASVMGPLAKINDWGSFKKQLQTLKNNGVYAITTDVWWGYVESAGDNQFDWSYYKTYANAVKEAGLKWVPIISTHKCGGNVGDDCNIPLPSWLSSKGSADEMQFKDESGYANSEALSPLWSGTGKQYDELYASFAENFAGYKSIIPKIYLSGGPSGELRYPSYYPAAGWSYPGRGKFQAYTETAKNAFRTAMNDKYGSLDKINAAWGTKLTSLSQINPPTDGDGFYTNGGYNSAYGKDFLSWYQSVLEKHLGVIGAAAHKNFDSVFGVRIGAKISGLHWQMNNPAMPHGTEQAGGYYDYNRLIQKFKDADLDLTFTCLEMSDSGTAPNYSLPSTLVDTVSSIANAKGVRLNGENALPTGGSGFQKIEEKITKFGYHGFTLLRINNLVNNDGSPTGELSGFKQYIISKAKPDNNGGTGNKVTIYYKKGFNSPYIHYRPAGGSWTAAPGVKMQDAEISGYAKITVDIGSASQLEAAFNDGNNNWDSNNTKNYSFSTGTSTYTPGNSGNAGTITSGAPAGANPGDGGGTTNKVTVYYKKGFNSPYIHYRPAGGSWTAAPGVKMQDAEISGYAKITVDIGSASQLEAAFNDGNNNWDSNNTKNYLFSTGTSTYTPGSNGAAGTIRTGAPSGSVLSVVTSTYATDLNEVTGPIQTEKLSGVSLNVSTSTYAPNSNGVEVTAQTEAPSGAFTSMDLGTLSNPTSLNTDWSKQSIYFIMTDRFSNGDPSNDNYGGFNSNNSDQRKWHGGDFQGIINKLDYIKNMGFTAIWITPVTMQKSEYAYHGYHTYDFYAVDGHLGTMDKLQELVRKAHDKNIAVMVDVVVNHTGDFQPGNGFAKAPFDKADWYHHNGDITDGDYNSNNQWKIENGDVAGLDDLNHENPATANELKNWIKWLLNETGIDGLRLDTVKHVPKGFLKDFDQAANTFTMGEIFHGDPAYVGDYTRYLDAALDFPMYYTIKDVFGHDQSMRKIKDRYSDDRYYRDAQTNGVFIDNHDVKRFLNDASGKPGANYDKWPQLKAALGFTLTSRGIPIIYQGTEQGYSGGDDPANRENMNFNANHDLYQYIAKLNYVRNNHPALQNGSQREKWVDDSFYSFQRSKNGDEAIVFINNSWNSQTRTIGNFDNLSNGTRLTNQLSNDSVQINNGSITVTLAPKEVKVFTK

SEQ ID NO: 404 - protein, 1167 aa, synthetic construct
AVADDFQASVMGPLAKINDWGSFKKQLQTLKNNGVYAITTDVWWGYVESAGDNQFDWSYYKTYANAVKEAGLKWVPIISTHKCGGNVGDDCNIPLPSWLSSKGSADEMQFKDESGYANSEALSPLWSGTGKQYDELYASFAENFAGYKSIIPKIYLSGGPSGELRYPSYYPAAGWSYPGRGKFQAYTETAKNAFRTAMNDKYGSLDKINAAWGTKLTSLSQINPPTDGDGFYTNGGYNSAYGKDFLSWYQSVLEKHLGVIGAAAHKNFDSVFGVRIGAKISGLHWQMNNPAMPHGTEQAGGYYDYNRLIQKFKDADLDLTFTCLEMSDSGTAPNYSLPSTLVDTVSSIANAKGVRLNGENALPTGGSGFQKIEEKITKFGYHGFTLLRINNLVNNDGSPTGELSGFKQYIISKAKPDNNGGTGNKVTIYYKKGFNSPYIHYRPAGGSWTAAPGVKMQDAEISGYAKITVDIGSASQLEAAFNDGNNNWDSNNTKNYSFSTGTSTYTPGNSGNAGTITSGAPAGANPGDGGGTTNKVTVYYKKGFNSPYIHYRPAGGSWTAAPGVKMQDAEISGYAKITVDIGSASQLEAAFNDGNNNWDSNNTKNYLFSTGTSTYTPGSNGAAGTIRTGAPSGSVLSVVTSTYATDLNEVTGPIQTEKLSGVSLNVSTSTYAPNSNGVEVTAQTEAPSGAFTSMDLGTLSNPTSLNTDWSKQSIYFIMTDRFSNGDPSNDNYGGFNSNNSDQRKWHGGDFQGIINKLDYIKNMGFTAIWITPVTMQKSEYAYHGYHTYDFYAVDGHLGTMDKLQELVRKAHDKNIAVMVDVVVNHTGDFQPGNGFAKAPFDKADWYHHNGDITDGDYNSNNQWKIENGDVAGLDDLNHENPATANELKNWIKWLLNETGIDGLRLDTVKHVPKGFLKDFDQAANTFTMGEIFHGDPAYVGDYTRYLDAALDFPMYYTIKDVFGHDQSMRKIKDRYSDDRYYRDAQTNGVFIDNHDVKRFLNDASGKPGANYDKWPQLKAALGFTLTSRGIPIIYQGTEQGYSGGDDPANRENMNFNANHDLYQYIAKLNYVRNNHPALQNGSQREKWVDDSFYSFQRSKNGDEAIVFINNSWNSQTRTIGNFDNLSNGTRLTNQLSNDSVQINNGSITVTLAPKEVKVFTKHHHHHH

SEQ ID NO: 405 - protein, 669 aa, synthetic construct
MKLNKIITTAGLSLGLLLPSIATATPTTFVHLFEWNWQDVAQECEQYLGPKGYAAVQVSPPNEHITGSQWWTRYQPVSYELQSRGGNRAQFIDMVNRCSAAGVDIYVDTLINHMAAGSGTGTAGNSFGNKSFPIYSPQDFHESCTINNSDYGNDRYRVQNCELVGLADLDTASNYVQNTIAAYINDLQAIGVKGFRFDASKHVAASDIQSLMAKVNGSPVVFQEVIDQGGEAVGASEYLSTGLVTEFKYSTELGNTFRNGSLAWLSNFGEGWGFMPSSSAVVFVDNHDNQRGHGGAGNVITFEDGRLYDLANVFMLAYPYGYPKVMSSYDFHGDTDAGGPNVPVHNNGNLECFASNWKCEHRWSYIAGGVDFRNNTADNWAVTNWWDNTNNQISFGRGSSGHMAINKEDSTLTATVQTDMASGQYCNVLKGELSADAKSCSGEVITVNSDGTINLNIGAWDAMAIHKNAKLNTSSASSTESDWQRTVIFINAQTQSGQDMFIRGGIDHAYANANLGRNCQTSNFECAMPIRHNNLKNVTTSPWKANDNYLDWYGIENGQSSEAEGSATDWTTNVWPAGWGAEKTVNTDGFGVTPLNIWGEHYWMLDVDMDCSKAVNGWFELKAFIKNGQGWETAIAQDNAPYTSTNHMAQCGKINKFEFNNSGVVIRSF

SEQ ID NO: 406 - protein, 651 aa, synthetic construct
TPTTFVHLFEWNWQDVAQECEQYLGPKGYAAVQVSPPNEHITGSQWWTRYQPVSYELQSRGGNRAQFIDMVNRCSAAGVDIYVDTLINHMAAGSGTGTAGNSFGNKSFPIYSPQDFHESCTINNSDYGNDRYRVQNCELVGLADLDTASNYVQNTIAAYINDLQAIGVKGFRFDASKHVAASDIQSLMAKVNGSPVVFQEVIDQGGEAVGASEYLSTGLVTEFKYSTELGNTFRNGSLAWLSNFGEGWGFMPSSSAVVFVDNHDNQRGHGGAGNVITFEDGRLYDLANVFMLAYPYGYPKVMSSYDFHGDTDAGGPNVPVHNNGNLECFASNWKCEHRWSYIAGGVDFRNNTADNWAVTNWWDNTNNQISFGRGSSGHMAINKEDSTLTATVQTDMASGQYCNVLKGELSADAKSCSGEVITVNSDGTINLNIGAWDAMAIHKNAKLNTSSASSTESDWQRTVIFINAQTQSGQDMFIRGGIDHAYANANLGRNCQTSNFECAMPIRHNNLKNVTTSPWKANDNYLDWYGIENGQSSEAEGSATDWTTNVWPAGWGAEKTVNTDGFGVTPLNIWGEHYWMLDVDMDCSKAVNGWFELKAFIKNGQGWETAIAQDNAPYTSTNHMAQCGKINKFEFNNSGVVIRSFHHHHHH

SEQ ID NO: 407 - protein, 692 aa, synthetic construct
MNKHLHFLSLLWLSMLMAFMTACSDDKNITDPAPEPEPPVEGQWTALTASPDTWDETKRADISYQLLLYSFADSDGDGYGDLNGVTQKLDYLNQLGVKALWLSPIHPCMSYHGYDVTDYTKVNPQLGTESDFDRLVTEAHNRGIKIYLDYVMNHTGTAHPWFTEASSSSESPYRNYYSFSEDPKTDIAAGKIAMITQEGAAGYNAAEWFQVSDETAAVKGLLKFTLDWSNAPSPILVVSTGTKADEDNPDTGTDNAKYLYYGEDICKKFYDKGNNIYELTVDFESTWGLLIRTSNASFWPSGTKYGASSSSEKLALNKDFKLTNAGNPANIMFDSQQITYFHSHFCTDWFADLNYGPVDQAGESPAYQAIADAAKGWIARGVDGLRLDAVKHIYHSETSEENPRFLKMFYEDMNAYYKQKGHTDDFYMIGEVLSEYDKVAPYYKGLPALFEFSFWYRLEWGINNSTGCYFAKDILSYQQKYANYRSDYIEATKLSNHDEDRTSSKLGKSADKCKLAAAVLLTSAGHPYIYYGEELGLYGTKDNGDEYVRSPMLWGDSYTTNYTDKTDATVSKNVKTVADQQADTHSLLNIYFSLTRLRNTYPALAEGNMTKHSVYNESQEKDYKPIAAWYMTKDNEKLLVIHNFGGTAMQLPLTDKIEKVLFVNGETQQNTDSDSYTLKLGGYASVVFKLGN

SEQ ID NO: 408 - protein, 676 aa, synthetic construct
CSDDKNITDPAPEPEPPVEGQWTALTASPDTWDETKRADISYQLLLYSFADSDGDGYGDLNGVTQKLDYLNQLGVKALWLSPIHPCMSYHGYDVTDYTKVNPQLGTESDFDRLVTEAHNRGIKIYLDYVMNHTGTAHPWFTEASSSSESPYRNYYSFSEDPKTDIAAGKIAMITQEGAAGYNAAEWFQVSDETAAVKGLLKFTLDWSNAPSPILVVSTGTKADEDNPDTGTDNAKYLYYGEDICKKFYDKGNNIYELTVDFESTWGLLIRTSNASFWPSGTKYGASSSSEKLALNKDFKLTNAGNPANIMFDSQQITYFHSHFCTDWFADLNYGPVDQAGESPAYQAIADAAKGWIARGVDGLRLDAVKHIYHSETSEENPRFLKMFYEDMNAYYKQKGHTDDFYMIGEVLSEYDKVAPYYKGLPALFEFSFWYRLEWGINNSTGCYFAKDILSYQQKYANYRSDYIEATKLSNHDEDRTSSKLGKSADKCKLAAAVLLTSAGHPYIYYGEELGLYGTKDNGDEYVRSPMLWGDSYTTNYTDKTDATVSKNVKTVADQQADTHSLLNIYFSLTRLRNTYPALAEGNMTKHSVYNESQEKDYKPIAAWYMTKDNEKLLVIHNFGGTAMQLPLTDKIEKVLFVNGETQQNTDSDSYTLKLGGYASVVFKLGNHHHHHH

SEQ ID NO: 409 - protein, 659 aa, synthetic construct
MFAKRFKTSLLPLFAGFLLLFHLVLAGPAAASAETANKSNELTAPSIKSGTILHAWNWSFNTLKHNMKDIHDAGYTAIQTSPINQVKEGNQGDKSMSNWYWLYQPTSYQIGNRYLGTEQEFKEMCAAAEEYGIKVIVDAVINHTTSDYAAISNEVKSIPNWTHGNTQIKNWSDRWDVTQNSLLGLYDWNTQNTQVQSYLKRFLDRALNDGADGFRFDAAKHIELPDDGSYGSQFWPNITNTSAEFQYGEILQDSASRDAAYANYMDVTASNYGHSIRSALKNRNLGVSNISHYASDVSADKLVTWVESHDTYANDDEESTWMSDDDIRLGWAVIASRSGSTPLFFSRPEGGGNGVRFPGKSQIGDRGSALFEDQAITAVNRFHNVMAGQPEELSNPNGNNQIFMNQRGSHGVVLANAGSSSVSINTATKLPDGRYDNKAGAGSFQVNDGKLTGTINARSVAVLYPDDIAKAPHVFLENYKTGVTHSFNDQLTITLRADANTTKAVYQINNGPETAFKDGDQFTIGKGDPFGKTYTIMLKGTNSDGVTRTEKYSFVKRDPASAKTIGYQNPNHWSQVNAYIYKHDGSRVIELTGSWPGKPMTKNADGIYTLTLPADTDTTNAKVIFNNGSAQVPGQNQPGFDYVLNGLYNDSGLSGSLPH

SEQ ID NO: 410 - protein, 634 aa, synthetic construct
SAETANKSNELTAPSIKSGTILHAWNWSFNTLKHNMKDIHDAGYTAIQTSPINQVKEGNQGDKSMSNWYWLYQPTSYQIGNRYLGTEQEFKEMCAAAEEYGIKVIVDAVINHTTSDYAAISNEVKSIPNWTHGNTQIKNWSDRWDVTQNSLLGLYDWNTQNTQVQSYLKRFLDRALNDGADGFRFDAAKHIELPDDGSYGSQFWPNITNTSAEFQYGEILQDSASRDAAYANYMDVTASNYGHSIRSALKNRNLGVSNISHYASDVSADKLVTWVESHDTYANDDEESTWMSDDDIRLGWAVIASRSGSTPLFFSRPEGGGNGVRFPGKSQIGDRGSALFEDQAITAVNRFHNVMAGQPEELSNPNGNNQIFMNQRGSHGVVLANAGSSSVSINTATKLPDGRYDNKAGAGSFQVNDGKLTGTINARSVAVLYPDDIAKAPHVFLENYKTGVTHSFNDQLTITLRADANTTKAVYQINNGPETAFKDGDQFTIGKGDPFGKTYTIMLKGTNSDGVTRTEKYSFVKRDPASAKTIGYQNPNHWSQVNAYIYKHDGSRVIELTGSWPGKPMTKNADGIYTLTLPADTDTTNAKVIFNNGSAQVPGQNQPGFDYVLNGLYNDSGLSGSLPHHHHHHH

SEQ ID NO: 411 - protein, 514 aa, synthetic construct
MIQKRKRTVSFRLVLMCTLLFVSLPITKTSAVNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 412 - protein, 489 aa, synthetic construct
VNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQKHHHHHH

SEQ ID NO: 413 - protein, 549 aa, synthetic construct
MLTFHRIIRKGWMFLLAFLLTALLFCPTGQPAKAAAPFNGTMMQYFEWYLPDDGTLWTKVANEANNLSSLGITALWLPPAYKGTSRSDVGYGVYDLYDLGEFNQKGAVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKSWGKWYVNTTNIDGFRLDAVKHIKFSFFPDWLSDVRSQTGKPLFTVGEYWSYDINKLHNYIMKTNGTMSLFDAPLHNKFYTASKSGGTFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWSITTRPWTDEFVRWTEPRLVAWP

SEQ ID NO: 414 - protein, 521 aa, synthetic construct
AAPFNGTMMQYFEWYLPDDGTLWTKVANEANNLSSLGITALWLPPAYKGTSRSDVGYGVYDLYDLGEFNQKGAVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKSWGKWYVNTTNIDGFRLDAVKHIKFSFFPDWLSDVRSQTGKPLFTVGEYWSYDINKLHNYIMKTNGTMSLFDAPLHNKFYTASKSGGTFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWSITTRPWTDEFVRWTEPRLVAWPHHHHHH

SEQ ID NO: 415 - protein, 530 aa, synthetic construct
QSLVSSNPHSNSTNTGDSASNLSINEVKYPVVYEIFIRSLYDSDGDGVGDINGVSQKVDYLRKLGIDAVWFMPFNEAVSYHGYDITDYYNVEKDYGTMEDLENMIQVLHENGIKVIMDLVINHTSDEHPWFKDAVENTTSSPYWDYYIMSLEDHSGQDHWHWKINSKGQKVWYFGLFGYNMPDLNHDSQKVREEVKKIVDFWISKGVDGFRIDAAKHIYGWSWDDGIQESAEYFEWFRDYVLSKKPDAILVGEVFSGNTYDLSLYPIPVFNFALMYSIRNYPEGQDGMIENNWVEESFLFLENHDLHRFFSHLQEHYKKFSESDYEFIKKRAALWYFLIFTLKGSPVIYYGGEIGTRGFKWHGPVYDEPVREPMQWYASGTGEGQTFWTKEVYKNAGITFGNADVDGCIYDDPYDGFSVEEQENDPKSLLNFIRFILNFRKDHDAILNGDQTIFRDWKNLIAFYRESSNEKLLVVLNPDPVWQNSFTFEENMTMILEVDFENFIWNESNVSFSAGESFTVDPMKAYIFKK

SEQ ID NO: 416 - protein, 536 aa, synthetic construct
QSLVSSNPHSNSTNTGDSASNLSINEVKYPVVYEIFIRSLYDSDGDGVGDINGVSQKVDYLRKLGIDAVWFMPFNEAVSYHGYDITDYYNVEKDYGTMEDLENMIQVLHENGIKVIMDLVINHTSDEHPWFKDAVENTTSSPYWDYYIMSLEDHSGQDHWHWKINSKGQKVWYFGLFGYNMPDLNHDSQKVREEVKKIVDFWISKGVDGFRIDAAKHIYGWSWDDGIQESAEYFEWFRDYVLSKKPDAILVGEVFSGNTYDLSLYPIPVFNFALMYSIRNYPEGQDGMIENNWVEESFLFLENHDLHRFFSHLQEHYKKFSESDYEFIKKRAALWYFLIFTLKGSPVIYYGGEIGTRGFKWHGPVYDEPVREPMQWYASGTGEGQTFWTKEVYKNAGITFGNADVDGCIYDDPYDGFSVEEQENDPKSLLNFIRFILNFRKDHDAILNGDQTIFRDWKNLIAFYRESSNEKLLVVLNPDPVWQNSFTFEENMTMILEVDFENFIWNESNVSFSAGESFTVDPMKAYIFKKHHHHHH

SEQ ID NO: 417 - protein, 551 aa, synthetic construct
MKKILSILLAGAMLAGCVLTGCSDSSSSKSDSSDGNTAKVADPIEGVKATASTDKYRNYYEIFVNSFCDSNGDETGDLQGIISQLDYLNDGDPNSGDDLGVDAIWLTPIMPSKSYHKYDVEDYYNIDPDFGTLDDFDKLIEECHKRGINVILDLVLNHASSKNPLFTKAVEEVADNKLDGNAEYFEIHKASYFDSNTQTISLGNGYACEANFSQEMPEWNLNSKKTREEFTKIAKFWLDRGVDGFRLDACKYFTNKETDGTEFLKWFYDTCKGIKEDVYMVGENWTDDSDIQELYKSGIDSQFAFKFSTSTGTIISNIISQGGMATAKKIMNYDNKMAESNPNAINAMFLSNHDQVRSGNALESQGLSSQKLAAAVYMLAPGNPFIYYGEEIGIKAPNTTNDAAYRTQMVFDSENLPDIYVNGIGDEPDVPTGGGVKQQLADKDSLLNYYRRIITIKNQNPEIARGRIVGTQGFDDKTVGAYYVEYEDSKLLIIHNFSKNDAKELTITDDMIKNATLRADLIPESSSKHTELKDGKISVPPQSTVIIKSAE

SEQ ID NO: 418 - protein, 536 aa, synthetic construct
CSDSSSSKSDSSDGNTAKVADPIEGVKATASTDKYRNYYEIFVNSFCDSNGDETGDLQGIISQLDYLNDGDPNSGDDLGVDAIWLTPIMPSKSYHKYDVEDYYNIDPDFGTLDDFDKLIEECHKRGINVILDLVLNHASSKNPLFTKAVEEVADNKLDGNAEYFEIHKASYFDSNTQTISLGNGYACEANFSQEMPEWNLNSKKTREEFTKIAKFWLDRGVDGFRLDACKYFTNKETDGTEFLKWFYDTCKGIKEDVYMVGENWTDDSDIQELYKSGIDSQFAFKFSTSTGTIISNIISQGGMATAKKIMNYDNKMAESNPNAINAMFLSNHDQVRSGNALESQGLSSQKLAAAVYMLAPGNPFIYYGEEIGIKAPNTTNDAAYRTQMVFDSENLPDIYVNGIGDEPDVPTGGGVKQQLADKDSLLNYYRRIITIKNQNPEIARGRIVGTQGFDDKTVGAYYVEYEDSKLLIIHNFSKNDAKELTITDDMIKNATLRADLIPESSSKHTELKDGKISVPPQSTVIIKSAEHHHHHH

SEQ ID NO: 419 - protein, 659 aa, synthetic construct
MFAKRLNTSFLPLFAGLLLLFHLILAGPVAVNAETANQSNEYTAPSIKSGTILHAWNWSFNTLKNNMKDIHDAGYTAIQTSPIKQVKEGNNGDKSMGNWYWLYQPTSYQIGNRYLGSEEEFKEMCAAAEEYGVKVIVDAVINHTTSDYAAISNEIKSISNWTHGNTQIKNWSDRWDVTQNSLLGLYDWNTQNTQVQSYLKRFLERALNDGADGFRYDAAKHIELPDDGNYGSQFWPNITNTSAEFQYGEILQDSASRDAAYANYMNVTASNYGHSIRSALKNRNLSVSNISHYASDVSADKLVTWVESHDTYANDDEESTWMSDDDIRLGWAVIASRSGSTPLFFSRPDGGGNGVRFPGKTQIGDRGSALFEDQAIVAVNTFHNVMAGQPEELSNPNGNNQIFMNQRGSKGVVLANAGSSSVSINASTKLPDGSYDNKAGTGSFQVRDGKLTGTINARSVAVLYPDDIANAPHVFLEDVKTGVTHSFNDQLTITLRADVNTAKAVYQINNGQETAFKDGDQLTIGKGDPFGTTYDITLTGTNSDGVTRAQEYSFVKRDPSAAKTIGYQNPNHWGHVNAYIYKHDGGRAIELTGSWPGKAMTKNADGIYTLTLPADTDTTNAKVIFNNGSAQVPGQNQPGFDYVQNGLYNNSGLSGSLPH

SEQ ID NO: 420 - protein, 632 aa, synthetic construct
ETANQSNEYTAPSIKSGTILHAWNWSFNTLKNNMKDIHDAGYTAIQTSPIKQVKEGNNGDKSMGNWYWLYQPTSYQIGNRYLGSEEEFKEMCAAAEEYGVKVIVDAVINHTTSDYAAISNEIKSISNWTHGNTQIKNWSDRWDVTQNSLLGLYDWNTQNTQVQSYLKRFLERALNDGADGFRYDAAKHIELPDDGNYGSQFWPNITNTSAEFQYGEILQDSASRDAAYANYMNVTASNYGHSIRSALKNRNLSVSNISHYASDVSADKLVTWVESHDTYANDDEESTWMSDDDIRLGWAVIASRSGSTPLFFSRPDGGGNGVRFPGKTQIGDRGSALFEDQAIVAVNTFHNVMAGQPEELSNPNGNNQIFMNQRGSKGVVLANAGSSSVSINASTKLPDGSYDNKAGTGSFQVRDGKLTGTINARSVAVLYPDDIANAPHVFLEDVKTGVTHSFNDQLTITLRADVNTAKAVYQINNGQETAFKDGDQLTIGKGDPFGTTYDITLTGTNSDGVTRAQEYSFVKRDPSAAKTIGYQNPNHWGHVNAYIYKHDGGRAIELTGSWPGKAMTKNADGIYTLTLPADTDTTNAKVIFNNGSAQVPGQNQPGFDYVQNGLYNNSGLSGSLPHHHHHHH

SEQ ID NO: 421 - protein, 505 aa, synthetic construct
MKRVFRALLIFALLLSVTTPVSAKTERTWQDERIYFIMVDRFNNGNPKNDYEVDVHDPKAYHGGDLQGIIDKLDYIKEMGFTAIWLTPIFANEKGGYHGYWIEDFYKVEEHFGTLDDFKRLVKEAHKRDMKVILDFVVNHTGYNHPWLNDPAKKDWFHEKKDIFNWANQQEVENGWLFGLPDLAQENPEVKTYLFDVAKWWIQETDIDGYRLDTVKHVPKWFWDEFAKEVKSVKQDFFLLGEVWHDDPRYVAEYGKHGIDALIDFPFYKEASTIFSNVDQSLEPLYNVWKRNVAFYERPYLLGTFLDNHDTVRFTRLALQNRINPVTRLKLGLTYLFSAPGIPIMYYGTEIALDGGEDPDNRRLMNFRTDKELIDYVTKLGELRAKLPSLRRGDFELLYEKDGMALFKRTYEKETTVIAINNTSKTQKVTLDGELEQGKELRGLLAGDLVRSKDGKYDIILDRETAEIYVLAPKTGLNIPFIAALVVVYTAFGLFLYFARKRKAS

SEQ ID NO: 422 - protein, 488 aa, synthetic construct
KTERTWQDERIYFIMVDRFNNGNPKNDYEVDVHDPKAYHGGDLQGIIDKLDYIKEMGFTAIWLTPIFANEKGGYHGYWIEDFYKVEEHFGTLDDFKRLVKEAHKRDMKVILDFVVNHTGYNHPWLNDPAKKDWFHEKKDIFNWANQQEVENGWLFGLPDLAQENPEVKTYLFDVAKWWIQETDIDGYRLDTVKHVPKWFWDEFAKEVKSVKQDFFLLGEVWHDDPRYVAEYGKHGIDALIDFPFYKEASTIFSNVDQSLEPLYNVWKRNVAFYERPYLLGTFLDNHDTVRFTRLALQNRINPVTRLKLGLTYLFSAPGIPIMYYGTEIALDGGEDPDNRRLMNFRTDKELIDYVTKLGELRAKLPSLRRGDFELLYEKDGMALFKRTYEKETTVIAINNTSKTQKVTLDGELEQGKELRGLLAGDLVRSKDGKYDIILDRETAEIYVLAPKTGLNIPFIAALVVVYTAFGLFLYFARKRKASHHHHHH

SEQ ID NO: 423 - protein, 483 aa, synthetic construct
ASLNGTLMQYFEWYMPNDGQHWKRLQNDSAYLAEHGITAVWIPPAYKGTSQDDVGYGAYDLYDLGEFHQKGTVRTKYGTKGELQSAINSLHSRDINVYGDVVINHKGGADATEYVTAVEVDPADRNRVTSGEQRIKAWTHFQFPGRGSTYSDFKWYWYHFDGTDWDESRKLNRIYKFQGKAWDWEVSNENGNYDYLMYADIDYDHPDVTAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPVKAVTFVDNHDTQPGQSLESTVQTWFKPLAYAFILTREAGYPQIFYGDMYGTKGASQREIPALKHKIEPILKARKQYAYGAQHDYFDHHNIVGWTREGDSSVANSGLAALITDGPGGTKRMYVGRQNAGETWHDITGNRSDSVVINAEGWGEFHVNGGSVSIYVQR

SEQ ID NO: 424 - protein, 489 aa, synthetic construct
ASLNGTLMQYFEWYMPNDGQHWKRLQNDSAYLAEHGITAVWIPPAYKGTSQDDVGYGAYDLYDLGEFHQKGTVRTKYGTKGELQSAINSLHSRDINVYGDVVINHKGGADATEYVTAVEVDPADRNRVTSGEQRIKAWTHFQFPGRGSTYSDFKWYWYHFDGTDWDESRKLNRIYKFQGKAWDWEVSNENGNYDYLMYADIDYDHPDVTAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPVKAVTFVDNHDTQPGQSLESTVQTWFKPLAYAFILTREAGYPQIFYGDMYGTKGASQREIPALKHKIEPILKARKQYAYGAQHDYFDHHNIVGWTREGDSSVANSGLAALITDGPGGTKRMYVGRQNAGETWHDITGNRSDSVVINAEGWGEFHVNGGSVSIYVQRHHHHHH

SEQ ID NO: 425 - protein, 555 aa, synthetic construct
MKKTWWKEGVAYQIYPRSFMDANGDGIGDLRGIIEKLDYLVELGVDIVWICPIYRSPNADNGYDISDYYAIMDEFGTMDDFDELLAQAHRRGLKVILDLVINHTSDEHPWFIESRSSRDNPKRDWYIWRDGKDGREPNNWESIFGGSAWQYDERTGQYYLHIFDVKQPDLNWENSEVRQALYEMVNWWLDKGIDGFRIDAISHIKKKPGLPDLPNPKGLKYVPSFAGHMNQPGIMEYLRELKEQTFARYDIMTVGEANGVTVDEAEQWVGEENGVFNMIFQFEHLGLWERRADGSIDVRRLKRTLTKWQKGLENRGWNALFLENHDLPRSVSTWGNDRDYWAESAKALGALYFFMQGTPFIYQGQEIGMTNVRFDDIRDYRDVSALRLYELERAKGRTHEEAMTIIWKTGRDNSRTPMQWSGASNAGFTTGTPWIKVNENYRTINVEAERRDPNSVWSFYRQMIQLRKANELFVYGTYDLLLENHPSIYAYTRTLGRDRALVVVNLSDRPSLYRYDGFRLQSSDLALSNYPVRPHKNATRFKLKPYEARVYIWKE

SEQ ID NO: 426 - protein, 560 aa, synthetic construct
KKTWWKEGVAYQIYPRSFMDANGDGIGDLRGIIEKLDYLVELGVDIVWICPIYRSPNADNGYDISDYYAIMDEFGTMDDFDELLAQAHRRGLKVILDLVINHTSDEHPWFIESRSSRDNPKRDWYIWRDGKDGREPNNWESIFGGSAWQYDERTGQYYLHIFDVKQPDLNWENSEVRQALYEMVNWWLDKGIDGFRIDAISHIKKKPGLPDLPNPKGLKYVPSFAGHMNQPGIMEYLRELKEQTFARYDIMTVGEANGVTVDEAEQWVGEENGVFNMIFQFEHLGLWERRADGSIDVRRLKRTLTKWQKGLENRGWNALFLENHDLPRSVSTWGNDRDYWAESAKALGALYFFMQGTPFIYQGQEIGMTNVRFDDIRDYRDVSALRLYELERAKGRTHEEAMTIIWKTGRDNSRTPMQWSGASNAGFTTGTPWIKVNENYRTINVEAERRDPNSVWSFYRQMIQLRKANELFVYGTYDLLLENHPSIYAYTRTLGRDRALVVVNLSDRPSLYRYDGFRLQSSDLALSNYPVRPHKNATRFKLKPYEARVYIWKEHHHHHH

SEQ ID NO: 427 - protein, 549 aa, synthetic construct
MLTFHRIIRKGWMFLLAFLLTASLFCPTGQPAKAAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPGVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTITSDGWGEFKVNGGSVSVWVPRKTTVSTITRPITTRPWTGEFVRWTEPRLVAWP

SEQ ID NO: 428 - protein, 521 aa, synthetic construct
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPGVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTITSDGWGEFKVNGGSVSVWVPRKTTVSTITRPITTRPWTGEFVRWTEPRLVAWPHHHHHH

SEQ ID NO: 429 - protein, 524 aa, synthetic construct
MRKNFKAFVALFAAILLFFSGCSSKQEAKAPKSEVIYQVMVDRFYNGDPSNDDPEVSKGMFDPTHTNWRMYWGGDLKGLTEKIPYIKGMGVTAIWISPVVDNINKLAVYNGEINAPYHGYWARDFKRVEEHFGTWEDFDNFVKVAHENGIKVILDFAPNHTSPADEENPDFAENGALYDDGKLLGTYSNDSLKLFHHNGSISNWNNLKELQDKNLFDLADLDQSNPIVDKYLKDSIKLWFNHEIDGVRLDAAKHMPMEWVKSFANTIYSIKKDVLLFGEWMLSGPTDPLYGYNIQFANTTGFSVLDFMLNGAIRDVFGKGYGFERLNDTLEDTNKDYENPYKLVTFIDNHDMPRFLSLNNDKDKLHEAIAFIMTTRGIPVIYYGTEQYLHNDTNGGNDPYNRPMMEKFDESTKAYTLIKELSRLRQLTPALQYGTTTARYISDDVYIYERQYGKDVVLVAINKGEKTTVKAVKTSLRKGIYKDYLKGLLKGVELKVTKGNGENLVQGLALPGDSVSVWTNVRVK

SEQ ID NO: 430 - protein, 509 aa, synthetic construct
CSSKQEAKAPKSEVIYQVMVDRFYNGDPSNDDPEVSKGMFDPTHTNWRMYWGGDLKGLTEKIPYIKGMGVTAIWISPVVDNINKLAVYNGEINAPYHGYWARDFKRVEEHFGTWEDFDNFVKVAHENGIKVILDFAPNHTSPADEENPDFAENGALYDDGKLLGTYSNDSLKLFHHNGSISNWNNLKELQDKNLFDLADLDQSNPIVDKYLKDSIKLWFNHEIDGVRLDAAKHMPMEWVKSFANTIYSIKKDVLLFGEWMLSGPTDPLYGYNIQFANTTGFSVLDFMLNGAIRDVFGKGYGFERLNDTLEDTNKDYENPYKLVTFIDNHDMPRFLSLNNDKDKLHEAIAFIMTTRGIPVIYYGTEQYLHNDTNGGNDPYNRPMMEKFDESTKAYTLIKELSRLRQLTPALQYGTTTARYISDDVYIYERQYGKDVVLVAINKGEKTTVKAVKTSLRKGIYKDYLKGLLKGVELKVTKGNGENLVQGLALPGDSVSVWTNVRVKHHHHHH

SEQ ID NO: 431 - protein, 564 aa, synthetic construct
MINNKIGGRNMVGRAVNRLAGRLTAVALTAAMAVSMLAGCAAGGKNTETAAKKEYKPSAMEQMNAKNDPNVIQDNYRTCYEVFVYSFFDSDGDGIGDLKGLTEKLDYIEGLGCNEIWMMPIMPSPSYHKYDITDYMNIDKQYGTLDDFDALITECHKRNINVIIDFVINHTSNEHPWFKAAADYIKSLPDGAEPDSSECPYVDYYNFSKTNTGGYNQLPGTNWYYESQFVDSMPDLNLQSEAVRGEIDKVTSFWLDRGVDGFRLDAVIYYNNNNQTETIDDLTWLVNNVKSKKADAYMVGEGWTTYREYAKYYKSGIDSMFNFDFSQQDGYIGKVLNGAANHGASTYGNALVDVENEIKKYTDSYIDAPFYTNHDMGRSAGYYNGDNAEEKTKMAQAMNLLMPGNAFLYYGEEIGMRGTANDETKRLAMRWSGDSKAKGMCVGPQNAEETEQTYDTLDKQMEDPYSIYNFVKQTISIRNAFPEIARGTNTFEKDLSNDNVCIFTREYNGEKAVLIFNPSKDEASVDVSSLGVNDAVAMLQTTKKAPSYKDGTAKLPAYSVLVLK

SEQ ID NO: 432 - protein, 531 aa, synthetic construct
CAAGGKNTETAAKKEYKPSAMEQMNAKNDPNVIQDNYRTCYEVFVYSFFDSDGDGIGDLKGLTEKLDYIEGLGCNEIWMMPIMPSPSYHKYDITDYMNIDKQYGTLDDFDALITECHKRNINVIIDFVINHTSNEHPWFKAAADYIKSLPDGAEPDSSECPYVDYYNFSKTNTGGYNQLPGTNWYYESQFVDSMPDLNLQSEAVRGEIDKVTSFWLDRGVDGFRLDAVIYYNNNNQTETIDDLTWLVNNVKSKKADAYMVGEGWTTYREYAKYYKSGIDSMFNFDFSQQDGYIGKVLNGAANHGASTYGNALVDVENEIKKYTDSYIDAPFYTNHDMGRSAGYYNGDNAEEKTKMAQAMNLLMPGNAFLYYGEEIGMRGTANDETKRLAMRWSGDSKAKGMCVGPQNAEETEQTYDTLDKQMEDPYSIYNFVKQTISIRNAFPEIARGTNTFEKDLSNDNVCIFTREYNGEKAVLIFNPSKDEASVDVSSLGVNDAVAMLQTTKKAPSYKDGTAKLPAYSVLVLKHHHHHH

SEQ ID NO: 433 - protein, 440 aa, synthetic construct
MARDTQTQLRNEMIYSVFVRNYSEAGNFAGVTADLQRIKDLGTDILWLLPINPIGEVNRKGTLGSPYAIKDYRGINPEYGTLADFKALTDRAHELGMKVMLDIVYNHTSPDSVLATEHPEWFYHDADGQLTNKVGDWSDVKDLDYGHHELWQYQIDTLLYWSQFVDGYRCDVAPLVPLDFWLEARKQVNAKYPETLWLAESAGSGFIEELRSQGYTGLSDSELYQAFDMTYDYDVFGDFKDYWQGRSTVERYVDLLQRQDATFPGNYVKMRFLENHDNARMMSLMHSKAEAVNNLTWIFMQRGIPLIYNGQEFLAEHQPSLFDRDTMVADRHGDVTPLIQKLVTIKQLPLLRAADYQLAVVEEGIVKITYRAAGEALTAWIPLKGQVTAVATKLAAGSYQNLLTDGPTEVVDGKLTVDGQPVLIKYVTNTAVTKVADQSN

SEQ ID NO: 434 - protein, 445 aa, synthetic construct
ARDTQTQLRNEMIYSVFVRNYSEAGNFAGVTADLQRIKDLGTDILWLLPINPIGEVNRKGTLGSPYAIKDYRGINPEYGTLADFKALTDRAHELGMKVMLDIVYNHTSPDSVLATEHPEWFYHDADGQLTNKVGDWSDVKDLDYGHHELWQYQIDTLLYWSQFVDGYRCDVAPLVPLDFWLEARKQVNAKYPETLWLAESAGSGFIEELRSQGYTGLSDSELYQAFDMTYDYDVFGDFKDYWQGRSTVERYVDLLQRQDATFPGNYVKMRFLENHDNARMMSLMHSKAEAVNNLTWIFMQRGIPLIYNGQEFLAEHQPSLFDRDTMVADRHGDVTPLIQKLVTIKQLPLLRAADYQLAVVEEGIVKITYRAAGEALTAWIPLKGQVTAVATKLAAGSYQNLLTDGPTEVVDGKLTVDGQPVLIKYVTNTAVTKVADQSNHHHHHH

SEQ ID NO: 435 - protein, 499 aa, synthetic construct
MMVAWWSLFLYGLQVAAPALAATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 436 - protein, 484 aa, synthetic construct
ATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSSHHHHHH

SEQ ID NO: 437 - protein, 484 aa, synthetic construct
LSAASWRTQSIYFLLTDRFGRTDNSTTATCNTGNEIYCGGSWQGIIDHLDYIEGMGFTAIWISPITEQLPQDTADGEAYHGYWQQKIYDVNSNFGTADNLKSLSDALHARGMYLMVDVVPDHMGYAGNGNDVDYSVFDPFDSSSYFHPYCLITDWDNLTMVEDCWEGDTIVSLPDLDTTETAVRTIWYDWVADLVSNYSVDGLRIDSVLEVQPDFFPGYNKASGVYCVGEIDNGNPASDCPYQKVLDGVLNYPIYWQLLYAFESSSGSISNLYNMIKSVASDCSDPTLLGNFIENHDNPRFAKYTSDYSQAKNVLSYIFLSDGIPIVYAGEEQHYAGGKVPYNREATWLSGYDTSAELYTWIATTNAIRKLAIAADSAYITYANDAFYTDSNTIAMAKGTSGSQVITVLSNKGSSGSSYTLTLSGSGYTSGTKLIEAYTCTSVTVDSSGDIPVPMASGLPRVLLPASVVDSSSLCGGSGRLYVE

SEQ ID NO: 438 - protein, 490 aa, synthetic construct
LSAASWRTQSIYFLLTDRFGRTDNSTTATCNTGNEIYCGGSWQGIIDHLDYIEGMGFTAIWISPITEQLPQDTADGEAYHGYWQQKIYDVNSNFGTADNLKSLSDALHARGMYLMVDVVPDHMGYAGNGNDVDYSVFDPFDSSSYFHPYCLITDWDNLTMVEDCWEGDTIVSLPDLDTTETAVRTIWYDWVADLVSNYSVDGLRIDSVLEVQPDFFPGYNKASGVYCVGEIDNGNPASDCPYQKVLDGVLNYPIYWQLLYAFESSSGSISNLYNMIKSVASDCSDPTLLGNFIENHDNPRFAKYTSDYSQAKNVLSYIFLSDGIPIVYAGEEQHYAGGKVPYNREATWLSGYDTSAELYTWIATTNAIRKLAIAADSAYITYANDAFYTDSNTIAMAKGTSGSQVITVLSNKGSSGSSYTLTLSGSGYTSGTKLIEAYTCTSVTVDSSGDIPVPMASGLPRVLLPASVVDSSSLCGGSGRLYVEHHHHHH

SEQ ID NO: 439 - protein, 492 aa, synthetic construct
MVSTALFLLAAAAGSARFAKAATPDEWRSRSIYQVLTDRFARGDGSPDAPCDTGARKYCGGNYRGLISQLDYIQGMGFDSVWISPITKQFEDDWNGAPYHGYWQTDLYALNEHFGTEEDLRALADELHARGMFLMVDVVINHNGWPGDAASIDYSQFNPFNSSDYYHPPCEINYDDQTSVEQCWLYTGANALPDLKTEDPHVSQVHNDWIADLVSKYSIDGLRIDTTKHVDKPAIGSFNDAAGVYAVGEVYHGDPAYTCPYQDWVDGVLNFPVYYPLIDAFKSPSGTMWSLVDNINKVFQTCNDPRLLGTFSENHDIPRFASYTQDLALAKNVLAFTILFDGIPIVYAGQEQQYSGDSDPYNREALWLSGFNTDAPLYKHIAACNRIRSHAVSNDDAYITTPTDIKYSDDHTLALVKGAVTTVLTNAGANAGETTVTVEATGYASGEQVTDVLSCESIAASDGGRLSVTLNQGLPRVFFPTDALAGSGLCEN

SEQ ID NO: 440 - protein, 471 aa, synthetic construct
MKFSISLSAAIVLFAAATSLASPLPQQQRYAKRATSDDWKSKAIYQLLTDRFGRADDSTSNCSNLSNYCGGTYEGITKHLDYISGMGFDAIWISPIPKNSDGGYHGYWATDFYQLNSNFGDESQLKTLIQAAHERGMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPPLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTANYDTSSDLYQFIKTVNSVRMKSNKTVYMDIYVGDNAYAFKHGDALVVLNNYGSGSTNQVSFSVSGKFDSGASLMDIVSNITTTVSSDGTVTFNLKDGLPAIFTSA

SEQ ID NO: 441 - protein, 444 aa, synthetic construct
ATSDDWKSKAIYQLLTDRFGRADDSTSNCSNLSNYCGGTYEGITKHLDYISGMGFDAIWISPIPKNSDGGYHGYWATDFYQLNSNFGDESQLKTLIQAAHERGMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPPLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTANYDTSSDLYQFIKTVNSVRMKSNKTVYMDIYVGDNAYAFKHGDALVVLNNYGSGSTNQVSFSVSGKFDSGASLMDIVSNITTTVSSDGTVTFNLKDGLPAIFTSAHHHHHH

SEQ ID NO: 442 - protein, 439 aa, synthetic construct
AAAADWKSRSIYQLVTDRFGRSDGSTSACGDLSNYCGGDYKGIQNQLDYIAGMGFDAIWISPIPENTDGGYHGYWAKDFEKLNTNFGSADDLKALVTAAHGKGMYVMLDVVANHAGPASGGDYSGFTFSSASNYHPQCTIDYDNQTSVEQCWVADDLPDINTEDDTIVSKLHSIVSDWVTTYDFDGIRIDTVKHIRKDFWSGYEEAAGVFATGEVFDGDAAYVGPYQDQLSSLINYPLYYAIRDVFSAGSGFSRISDMLSTIKSNFKDPSVLTTFVDNQDNARFLSVKSDMSLYKNALAFTILTEGIPVVYYGTEQGFKGGDDPKNREVLWTSNYDTSSDLYKFIKIVNNDVRQKSDKTVTLDVDVGTNTYAFTHGKNLIVVNNYGSGSTESVTVKVGDSVADGTKLVDAVSNITATVSGGSITFSLKDGLPALFVPSS

SEQ ID NO: 443 - protein, 445 aa, synthetic construct
AAAADWKSRSIYQLVTDRFGRSDGSTSACGDLSNYCGGDYKGIQNQLDYIAGMGFDAIWISPIPENTDGGYHGYWAKDFEKLNTNFGSADDLKALVTAAHGKGMYVMLDVVANHAGPASGGDYSGFTFSSASNYHPQCTIDYDNQTSVEQCWVADDLPDINTEDDTIVSKLHSIVSDWVTTYDFDGIRIDTVKHIRKDFWSGYEEAAGVFATGEVFDGDAAYVGPYQDQLSSLINYPLYYAIRDVFSAGSGFSRISDMLSTIKSNFKDPSVLTTFVDNQDNARFLSVKSDMSLYKNALAFTILTEGIPVVYYGTEQGFKGGDDPKNREVLWTSNYDTSSDLYKFIKIVNNDVRQKSDKTVTLDVDVGTNTYAFTHGKNLIVVNNYGSGSTESVTVKVGDSVADGTKLVDAVSNITATVSGGSITFSLKDGLPALFVPSSHHHHHH

SEQ ID NO: 444 - protein, 460 aa, synthetic construct
MKLTASLTLLTQALAVLGADTNSWKSRTIYFALTDRVARSASDNGGDGCGQLQDYCGGTFKGLEGKLDYIKGMGFDAIWITPVVQNSARGYHGYWASNLYATNSHYGTSDELKGLVNAAHGKGIYIMVDVVANHVGNGPLNEMQPAPLNQGSSYHPACGINYNDQHSIETCRVASDLPDLDTTDPKIRTLYKDWIKWLMSTYKFDGVRIDTVKHVEKDFWPDFAWASGSYTIGEVFSGDPNYVAGYSKLMGGLLNYPVYFPLNRFYQQQNSSQALVDMHNQIGSLVPDPTTLGTFLDNHDNPRFLSQKNDVSLFKNALTYVLLARGIPIVYYGSEQAYAGGGDPQNREDLWRSRFNTNSDMYKFFQALGGVRKSHGGLPGNDHVHLFVESDAYAWSRQDGAVMALTSNIGKGQQRQFCFFTQKNNKTWRGIFDGKTYTSGGDGKLCATVNNGEPIVFVAQ

SEQ ID NO: 445 - protein, 448 aa, synthetic construct
ADTNSWKSRTIYFALTDRVARSASDNGGDGCGQLQDYCGGTFKGLEGKLDYIKGMGFDAIWITPVVQNSARGYHGYWASNLYATNSHYGTSDELKGLVNAAHGKGIYIMVDVVANHVGNGPLNEMQPAPLNQGSSYHPACGINYNDQHSIETCRVASDLPDLDTTDPKIRTLYKDWIKWLMSTYKFDGVRIDTVKHVEKDFWPDFAWASGSYTIGEVFSGDPNYVAGYSKLMGGLLNYPVYFPLNRFYQQQNSSQALVDMHNQIGSLVPDPTTLGTFLDNHDNPRFLSQKNDVSLFKNALTYVLLARGIPIVYYGSEQAYAGGGDPQNREDLWRSRFNTNSDMYKFFQALGGVRKSHGGLPGNDHVHLFVESDAYAWSRQDGAVMALTSNIGKGQQRQFCFFTQKNNKTWRGIFDGKTYTSGGDGKLCATVNNGEPIVFVAQHHHHHH